SEQUENCE LISTING (518 sequences)

SEQ ID NO: 1 - protein, 119 aa, Homo sapiens
EDELQVIQPEKSVSVAAGESATLRCAMTSLIPVGPIMWFRGAGAGRELIYNQKEGHFPRVTTVSELTKRNNLDFSISISNITPADAGTYYCVKFRKGSPDDVEFKSGAGTELSVRAKPS

SEQ ID NO: 2 - protein, 119 aa, Homo sapiens
EEELQVIQPDKSISVAAGESATLHCTVTSLIPVGPIQWFRGAGPGRELIYNQKEGHFPRVTTVSDLTKRNNMDFSIRISNITPADAGTYYCVKFRKGSPDHVEFKSGAGTELSVRAKPS

SEQ ID NO: 3 - protein, 119 aa, Homo sapiens
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 4 - protein, 118 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 5 - protein, 118 aa, Homo sapiens
EEELQVIQPDKSVSVAAGESAILLCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 6 - protein, 119 aa, Homo sapiens
EEGLQVIQPDKSVSVAAGESAILHCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 7 - protein, 119 aa, Homo sapiens
EEELQVIQPDKFVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 8 - protein, 119 aa, Homo sapiens
EEELQVIQPDKSVLVAAGETATLRCTATSLIPVGPIQWFRGAGPGRELIYNQKEGHFPRVTTVSDLTKRNNMDFPIRIGNITPADAGTYYCVKFRKGSPDDVEFKSGAGTELSVRAKPS

SEQ ID NO: 9 - protein, 118 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRGKPS

SEQ ID NO: 10 - protein, 118 aa, Homo sapiens
EEELQVIQPDKSVLVAAGETATLRCTATSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 11 - protein, 119 aa, Homo sapiens
EEELQVIQPDKSVLVAAGETATLRCTATSLIPVGPIQWFRGAGPGRELIYNQKEGHFPRVTTVSDLTKRNNMDFSIRISNITPADAGTYYCVKFRKGSPDDVEFKSGAGTELSVRAKPS

SEQ ID NO: 12 - protein, 119 aa, synthetic construct
EDELQVIQPEKSVSVAAGESATLRCAVTSLIPVGPIQWFRGAGAGRELIYNQKEGHFPRVTTVSELTKRNNLDFSISISNITPADAGTYYCVKFRKGSPDDVEFKSGAGTELSVRAKPS

SEQ ID NO: 13 - protein, 119 aa, synthetic construct
EDELQVIQPEKSVSVAAGESATLRCAVTSLIPVGPIQWFRGAGPGRELIYNQKEGHFPRVTTVSELTKRNNLDFSISISNITPADAGTYYCVKFRKGSPDDVEFKSGAGTELSVRAKPS

SEQ ID NO: 14 - protein, 119 aa, synthetic construct
EDELQVIQPEKSVSVAAGESATLRCAVTSLIPVGPIQWFRGAGAGRELIYNQKEGHFPRVTTVSELTKRNNMDFSISISNITPADAGTYYCVKFRKGSPDDVEFKSGAGTELSVRAKPS

SEQ ID NO: 15 - protein, 119 aa, synthetic construct
EDELQVIQPEKSVSVAAGESATLRCAVTSLIPVGPIQWFRGAGPGRELIYNQKEGHFPRVTTVSELTKRNNMDFSISISNITPADAGTYYCVKFRKGSPDDVEFKSGAGTELSVRAKPS

SEQ ID NO: 16 - protein, 119 aa, synthetic construct
EDELQVIQPEKSVSVAAGESATLRCTVTSLIPVGPIQWFRGAGAGRELIYNQKEGHFPRVTTVSESTKRNNLDFSISISNITPADAGTYYCVKFRKGSPDDVEFKSGAGTELSVRAKPS

SEQ ID NO: 17 - protein, 119 aa, synthetic construct
EEELQVIQPDKSISVAAGESATLHCTVTSLIPVGPIQWFRGAGPGRELIYNQKEGHFPRVTTVSDLTKRNNMDFSIRISNITPADAGTYYCVKFRKGSPDDVEFKSGAGTELSVRAKPS

SEQ ID NO: 18 - protein, 119 aa, Homo sapiens
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPS

SEQ ID NO: 19 - protein, 119 aa, synthetic construct
EEELQMIQPEKLLLVTVGKTATLHCTVTSLLPVGPVQWFRGVGPGRELIYNQKEGHFPRVTTVSDLTKRNNMDFSIRISSITPADVGTYYCVKFRKGSPENVEFKSGPGTEMALGAKPS

SEQ ID NO: 20 - protein, 119 aa, synthetic construct
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPEDVEFKSGPGTEMALGAKPS

SEQ ID NO: 21 - protein, 119 aa, synthetic construct
EEELQMIQPEKLLLVTVGKTATLHCTVTSLLPVGPVQWFRGVGPGRELIYNQKEGHFPRVTTVSDLTKRNNMDFSIRISSITPADVGTYYCVKFRKGSPEDVEFKSGPGTEMALGAKPS

SEQ ID NO: 22 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNHKEGHFPRVTTVSDLTKRNNMDFSIRIGXITPADAGTYYCVKFRKGSPDDVEFKSGAGTELSVRAKPS

SEQ ID NO: 23 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNHKEGHFPRVTTVSESTKRENMDFSISISXITPADAGTYYCVKFRKGSPDTEFKSGAGTELSVRAKPS

SEQ ID NO: 24 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILLCTVTSLXPVGPIXWFRGAGPARELIYNHKEGHFPRVTTVSESTKRENMDFSISISXITPADAGTYYCVKFRKGSPDTEFKSGAGTELSVRAKPS

SEQ ID NO: 25 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEGLQVIQPDKSVSVAAGESAILHCTATSLXPVGPIXWFRGAGPGRELIYNHKEGHFPRVTTVSDLTKRNNMDFSIRIGXITPADAGTYYCVKFRKGSPDDVEFKSGAGTELSVRAKPS

SEQ ID NO: 26 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKFVLVAAGETATLRCTATSLXPVGPIXWFRGAGPGRELIYNHKEGHFPRVTTVSDLTKRNNMDFSIRIGXITPADAGTYYCVKFRKGSPDDVEFKSGAGTELSVRAKPS

SEQ ID NO: 27 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNHKEGHFPRVTTVSDLTKRNNMDFPIRIGXITPADAGTYYCVKFRKGSPDDVEFKSGAGTELSVRAKPS

SEQ ID NO: 28 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNHKEGHFPRVTTVSESTKRENMDFSISISXITPADAGTYYCVKFRKGSPDTEFKSGAGTELSVRGKPS

SEQ ID NO: 29 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPARELIYNHKEGHFPRVTTVSESTKRENMDFSISISXITPADAGTYYCVKFRKGSPDTEFKSGAGTELSVRAKPS

SEQ ID NO: 30 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNHKEGHFPRVTTVSDLTKRNNMDFSIRISXITPADAGTYYCVKFRKGSPDDVEFKSGAGTELSVRAKPS

SEQ ID NO: 31 - protein, 119 aa, synthetic construct
  VARIANT 26: note = X is alanine or threonine.
  VARIANT 27: note = X is valine or alanine.
  VARIANT 31: note = X is isoleucine, glutamic acid or aspartic acid.
  VARIANT 37: note = X is glutamine or histidine.
  VARIANT 44: note = X is alanine or proline.
  VARIANT 66: note = X is leucine or serine.
  VARIANT 72: note = X is leucine or methionine.
  VARIANT 80: note = X is asparagine, alanine, serine or glutamine.
EDELQVIQPEKSVSVAAGESATLRCXXTSLXPVGPIXWFRGAGXGRELIYNHKEGHFPRVTTVSEXTKRNNXDFSISISXITPADAGTYYCVKFRKGSPDDVEFKSGAGTELSVRAKPS

SEQ ID NO: 32 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine or glutamine.
  VARIANT 101: note = X is aspartic acid.
EEELQVIQPDKSISVAAGESATLHCTVTSLXPVGPIXWFRGAGPGRELIYNHKEGHFPRVTTVSDLTKRNNMDFSIRISXITPADAGTYYCVKFRKGSPDXVEFKSGAGTELSVRAKPS

SEQ ID NO: 33 - protein, 119 aa, synthetic construct
  VARIANT 31: note = X is isoleucine, glutamic acid or aspartic acid.
  VARIANT 37: note = X is leucine, glutamine or histidine.
  VARIANT 101: note = B is asparagine or aspartic acid.
EEELQMIQPEKLLLVTVGKTATLHCTVTSLXPVGPVXWFRGVGPGRELIYNHKEGHFPRVTTVSDLTKRNNMDFSIRISSITPADVGTYYCVKFRKGSPEBVEFKSGPGTEMALGAKPS

SEQ ID NO: 34 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNQKEGHFPRVTTVSDLTHRNNMDFSIRIGXITPADAGTYYCVKFRKGSPDDVEFKSGAGTELSVRAKPS

SEQ ID NO: 35 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQKEGHFPRVTTVSESTHRENMDFSISISXITPADAGTYYCVKFRKGSPDTEFKSGAGTELSVRAKPS

SEQ ID NO: 36 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILLCTVTSLXPVGPIXWFRGAGPARELIYNQKEGHFPRVTTVSESTHRENMDFSISISXITPADAGTYYCVKFRKGSPDTEFKSGAGTELSVRAKPS

SEQ ID NO: 37 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEGLQVIQPDKSVSVAAGESAILHCTATSLXPVGPIXWFRGAGPGRELIYNQKEGHFPRVTTVSDLTHRNNMDFSIRIGXITPADAGTYYCVKFRKGSPDDVEFKSGAGTELSVRAKPS

SEQ ID NO: 38 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKFVLVAAGETATLRCTATSLXPVGPIXWFRGAGPGRELIYNQKEGHFPRVTTVSDLTHRNNMDFSIRIGXITPADAGTYYCVKFRKGSPDDVEFKSGAGTELSVRAKPS

SEQ ID NO: 39 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNQKEGHFPRVTTVSDLTHRNNMDFPIRIGXITPADAGTYYCVKFRKGSPDDVEFKSGAGTELSVRAKPS

SEQ ID NO: 40 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQKEGHFPRVTTVSESTHRENMDFSISISXITPADAGTYYCVKFRKGSPDTEFKSGAGTELSVRGKPS

SEQ ID NO: 41 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPARELIYNQKEGHFPRVTTVSESTHRENMDFSISISXITPADAGTYYCVKFRKGSPDTEFKSGAGTELSVRAKPS

SEQ ID NO: 42 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNQKEGHFPRVTTVSDLTHRNNMDFSIRISXITPADAGTYYCVKFRKGSPDDVEFKSGAGTELSVRAKPS

SEQ ID NO: 43 - protein, 119 aa, synthetic construct
  VARIANT 26: note = X is alanine or threonine.
  VARIANT 27: note = X is valine or alanine.
  VARIANT 31: note = X is isoleucine, glutamic acid or aspartic acid.
  VARIANT 37: note = X is glutamine or histidine.
  VARIANT 44: note = X is alanine or proline.
  VARIANT 66: note = X is leucine or serine.
  VARIANT 72: note = X is leucine or methionine.
  VARIANT 80: note = X is asparagine, alanine, serine or glutamine.
EDELQVIQPEKSVSVAAGESATLRCXXTSLXPVGPIXWFRGAGXGRELIYNQKEGHFPRVTTVSEXTHRNNXDFSISISXITPADAGTYYCVKFRKGSPDDVEFKSGAGTELSVRAKPS

SEQ ID NO: 44 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine or glutamine.
  VARIANT 101: note = X is aspartic acid.
EEELQVIQPDKSISVAAGESATLHCTVTSLXPVGPIXWFRGAGPGRELIYNQKEGHFPRVTTVSDLTHRNNMDFSIRISXITPADAGTYYCVKFRKGSPDXVEFKSGAGTELSVRAKPS

SEQ ID NO: 45 - protein, 119 aa, synthetic construct
  VARIANT 31: note = X is isoleucine, glutamic acid or aspartic acid.
  VARIANT 37: note = X is leucine, glutamine or histidine.
  VARIANT 101: note = B is asparagine or aspartic acid.
EEELQMIQPEKLLLVTVGKTATLHCTVTSLXPVGPVXWFRGVGPGRELIYNQKEGHFPRVTTVSDLTHRNNMDFSIRISSITPADVGTYYCVKFRKGSPEBVEFKSGPGTEMALGAKPS

SEQ ID NO: 46 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNQHEGHFPRVTTVSDLTKRNNMDFSIRIGXITPADAGTYYCVKFRKGSPDDVEFKSGAGTELSVRAKPS

SEQ ID NO: 47 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPS

SEQ ID NO: 48 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILLCTVTSLXPVGPIXWFRGAGPARELIYNQHEGHFPRVTTVSESTKRENMDFSISISXITPADAGTYYCVKFRKGSPDTEFKSGAGTELSVRAKPS

SEQ ID NO: 49 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEGLQVIQPDKSVSVAAGESAILHCTATSLXPVGPIXWFRGAGPGRELIYNQHEGHFPRVTTVSDLTKRNNMDFSIRIGXITPADAGTYYCVKFRKGSPDDVEFKSGAGTELSVRAKPS

SEQ ID NO: 50 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKFVLVAAGETATLRCTATSLXPVGPIXWFRGAGPGRELIYNQHEGHFPRVTTVSDLTKRNNMDFSIRIGXITPADAGTYYCVKFRKGSPDDVEFKSGAGTELSVRAKPS

SEQ ID NO: 51 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNQHEGHFPRVTTVSDLTKRNNMDFPIRIGXITPADAGTYYCVKFRKGSPDDVEFKSGAGTELSVRAKPS

SEQ ID NO: 52 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRGKPS

SEQ ID NO: 53 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPARELIYNQHEGHFPRVTTVSESTKRENMDFSISISXITPADAGTYYCVKFRKGSPDTEFKSGAGTELSVRAKPS

SEQ ID NO: 54 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNQHEGHFPRVTTVSDLTKRNNMDFSIRISXITPADAGTYYCVKFRKGSPDDVEFKSGAGTELSVRAKPS

SEQ ID NO: 55 - protein, 119 aa, synthetic construct
  VARIANT 26: note = X is alanine or threonine.
  VARIANT 27: note = X is valine or alanine.
  VARIANT 31: note = X is isoleucine, glutamic acid or aspartic acid.
  VARIANT 37: note = X is glutamine or histidine.
  VARIANT 44: note = X is alanine or proline.
  VARIANT 66: note = X is leucine or serine.
  VARIANT 72: note = X is leucine or methionine.
  VARIANT 80: note = X is asparagine, alanine, serine or glutamine.
EDELQVIQPEKSVSVAAGESATLRCXXTSLXPVGPIXWFRGAGXGRELIYNQHEGHFPRVTTVSEXTKRNNXDFSISISXITPADAGTYYCVKFRKGSPDDVEFKSGAGTELSVRAKPS

SEQ ID NO: 56 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine or glutamine.
  VARIANT 101: note = X is aspartic acid.
EEELQVIQPDKSISVAAGESATLHCTVTSLXPVGPIXWFRGAGPGRELIYNQHEGHFPRVTTVSDLTKRNNMDFSIRISXITPADAGTYYCVKFRKGSPDXVEFKSGAGTELSVRAKPS

SEQ ID NO: 57 - protein, 119 aa, synthetic construct
  VARIANT 31: note = X is isoleucine, glutamic acid or aspartic acid.
  VARIANT 37: note = X is leucine, glutamine or histidine.
  VARIANT 101: note = B is asparagine or aspartic acid.
EEELQMIQPEKLLLVTVGKTATLHCTVTSLXPVGPVXWFRGVGPGRELIYNQHEGHFPRVTTVSDLTKRNNMDFSIRISSITPADVGTYYCVKFRKGSPEBVEFKSGPGTEMALGAKPS

SEQ ID NO: 58 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNQKEGHFPRVTTVSDLTKHNNMDFSIRIGXITPADAGTYYCVKFRKGSPDDVEFKSGAGTELSVRAKPS

SEQ ID NO: 59 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPS

SEQ ID NO: 60 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILLCTVTSLXPVGPIXWFRGAGPARELIYNQKEGHFPRVTTVSESTKHENMDFSISISXITPADAGTYYCVKFRKGSPDTEFKSGAGTELSVRAKPS

SEQ ID NO: 61 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEGLQVIQPDKSVSVAAGESAILHCTATSLXPVGPIXWFRGAGPGRELIYNQKEGHFPRVTTVSDLTKHNNMDFSIRIGXITPADAGTYYCVKFRKGSPDDVEFKSGAGTELSVRAKPS

SEQ ID NO: 62 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKFVLVAAGETATLRCTATSLXPVGPIXWFRGAGPGRELIYNQKEGHFPRVTTVSDLTKHNNMDFSIRIGXITPADAGTYYCVKFRKGSPDDVEFKSGAGTELSVRAKPS

SEQ ID NO: 63 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNQKEGHFPRVTTVSDLTKHNNMDFPIRIGXITPADAGTYYCVKFRKGSPDDVEFKSGAGTELSVRAKPS

SEQ ID NO: 64 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRGKPS

SEQ ID NO: 65 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPARELIYNQKEGHFPRVTTVSESTKHENMDFSISISXITPADAGTYYCVKFRKGSPDTEFKSGAGTELSVRAKPS

SEQ ID NO: 66 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNQKEGHFPRVTTVSDLTKHNNMDFSIRISXITPADAGTYYCVKFRKGSPDDVEFKSGAGTELSVRAKPS

SEQ ID NO: 67 - protein, 119 aa, synthetic construct
  VARIANT 26: note = X is alanine or threonine.
  VARIANT 27: note = X is valine or alanine.
  VARIANT 31: note = X is isoleucine, glutamic acid or aspartic acid.
  VARIANT 37: note = X is glutamine or histidine.
  VARIANT 44: note = X is alanine or proline.
  VARIANT 66: note = X is leucine or serine.
  VARIANT 72: note = X is leucine or methionine.
  VARIANT 80: note = X is asparagine, alanine, serine or glutamine.
EDELQVIQPEKSVSVAAGESATLRCXXTSLXPVGPIXWFRGAGXGRELIYNQKEGHFPRVTTVSEXTKHNNXDFSISISXITPADAGTYYCVKFRKGSPDDVEFKSGAGTELSVRAKPS

SEQ ID NO: 68 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine or glutamine.
  VARIANT 101: note = X is aspartic acid.
EEELQVIQPDKSISVAAGESATLHCTVTSLXPVGPIXWFRGAGPGRELIYNQKEGHFPRVTTVSDLTKHNNMDFSIRISXITPADAGTYYCVKFRKGSPDXVEFKSGAGTELSVRAKPS

SEQ ID NO: 69 - protein, 119 aa, synthetic construct
  VARIANT 31: note = X is isoleucine, glutamic acid or aspartic acid.
  VARIANT 37: note = X is leucine, glutamine or histidine.
  VARIANT 101: note = B is asparagine or aspartic acid.
EEELQMIQPEKLLLVTVGKTATLHCTVTSLXPVGPVXWFRGVGPGRELIYNQKEGHFPRVTTVSDLTKHNNMDFSIRISSITPADVGTYYCVKFRKGSPEBVEFKSGPGTEMALGAKPS

SEQ ID NO: 70 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNHKEGHFPRVTTVSDLTHRNNMDFSIRIGXITPADAGTYYCVKFRKGSPDDVEFKSGAGTELSVRAKPS

SEQ ID NO: 71 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNHKEGHFPRVTTVSESTHRENMDFSISISXITPADAGTYYCVKFRKGSPDTEFKSGAGTELSVRAKPS

SEQ ID NO: 72 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILLCTVTSLXPVGPIXWFRGAGPARELIYNHKEGHFPRVTTVSESTHRENMDFSISISXITPADAGTYYCVKFRKGSPDTEFKSGAGTELSVRAKPS

SEQ ID NO: 73 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEGLQVIQPDKSVSVAAGESAILHCTATSLXPVGPIXWFRGAGPGRELIYNHKEGHFPRVTTVSDLTHRNNMDFSIRIGXITPADAGTYYCVKFRKGSPDDVEFKSGAGTELSVRAKPS

SEQ ID NO: 74 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKFVLVAAGETATLRCTATSLXPVGPIXWFRGAGPGRELIYNHKEGHFPRVTTVSDLTHRNNMDFSIRIGXITPADAGTYYCVKFRKGSPDDVEFKSGAGTELSVRAKPS

SEQ ID NO: 75 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNHKEGHFPRVTTVSDLTHRNNMDFPIRIGXITPADAGTYYCVKFRKGSPDDVEFKSGAGTELSVRAKPS

SEQ ID NO: 76 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNHKEGHFPRVTTVSESTHRENMDFSISISXITPADAGTYYCVKFRKGSPDTEFKSGAGTELSVRGKPS

SEQ ID NO: 77 - protein, 118 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPARELIYNHKEGHFPRVTTVSESTHRENMDFSISISXITPADAGTYYCVKFRKGSPDTEFKSGAGTELSVRAKPS

SEQ ID NO: 78 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVLVAAGETATLRCTATSLXPVGPIXWFRGAGPGRELIYNHKEGHFPRVTTVSDLTHRNNMDFSIRISXITPADAGTYYCVKFRKGSPDDVEFKSGAGTELSVRAKPS

SEQ ID NO: 79 - protein, 119 aa, synthetic construct
  VARIANT 26: note = X is alanine or threonine.
  VARIANT 27: note = X is valine or alanine.
  VARIANT 31: note = X is isoleucine, glutamic acid or aspartic acid.
  VARIANT 37: note = X is glutamine or histidine.
  VARIANT 44: note = X is alanine or proline.
  VARIANT 66: note = X is leucine or serine.
  VARIANT 72: note = X is leucine or methionine.
  VARIANT 80: note = X is asparagine, alanine, serine or glutamine.
EDELQVIQPEKSVSVAAGESATLRCXXTSLXPVGPIXWFRGAGXGRELIYNHKEGHFPRVTTVSEXTHRNNXDFSISISXITPADAGTYYCVKFRKGSPDDVEFKSGAGTELSVRAKPS

SEQ ID NO: 80 - protein, 119 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine or glutamine.
  VARIANT 101: note = X is aspartic acid.
EEELQVIQPDKSISVAAGESATLHCTVTSLXPVGPIXWFRGAGPGRELIYNHKEGHFPRVTTVSDLTHRNNMDFSIRISXITPADAGTYYCVKFRKGSPDXVEFKSGAGTELSVRAKPS

SEQ ID NO: 81 - protein, 119 aa, synthetic construct
  VARIANT 37: note = X is leucine, glutamine or histidine.
  VARIANT 101: note = B is asparagine or aspartic acid.
  VARIANT 31: note = X is isoleucine, glutamic acid or aspartic acid.
EEELQMIQPEKLLLVTVGKTATLHCTVTSLXPVGPVXWFRGVGPGRELIYNHKEGHFPRVTTVSDLTHRNNMDFSIRISSITPADVGTYYCVKFRKGSPEBVEFKSGPGTEMALGAKPS

SEQ ID NO: 82 - protein, 256 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPS

SEQ ID NO: 83 - protein, 256 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPS

SEQ ID NO: 84 - protein, 256 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNHKEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNHKEGHFPRVTTVSESTKRENMDFSISISXITPADAGTYYCVKFRKGSPDTEFKSGAGTELSVRAKPS

SEQ ID NO: 85 - protein, 256 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQKEGHFPRVTTVSESTHRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTHRENMDFSISISXITPADAGTYYCVKFRKGSPDTEFKSGAGTELSVRAKPS

SEQ ID NO: 86 - protein, 256 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPS

SEQ ID NO: 87 - protein, 256 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPS

SEQ ID NO: 88 - protein, 256 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNHKEGHFPRVTTVSESTKRENMDFSISISXITPADAGTYYCVKFRKGSPDTEFKSGAGTELSVRAKPS

SEQ ID NO: 89 - protein, 256 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNHKEGHFPRVTTVSESTKRENMDFSISISXITPADAGTYYCVKFRKGSPDTEFKSGAGTELSVRAKPS

SEQ ID NO: 90 - protein, 256 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTHRENMDFSISISXITPADAGTYYCVKFRKGSPDTEFKSGAGTELSVRAKPS

SEQ ID NO: 91 - protein, 256 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTHRENMDFSISISXITPADAGTYYCVKFRKGSPDTEFKSGAGTELSVRAKPS

SEQ ID NO: 92 - protein, 258 aa, synthetic construct
  VARIANT 26: note = X is alanine or threonine.
  VARIANT 27: note = X is valine or alanine.
  VARIANT 31: note = X is isoleucine, glutamic acid or aspartic acid.
  VARIANT 37: note = X is glutamine or histidine.
  VARIANT 44: note = X is alanine or proline.
  VARIANT 66: note = X is leucine or serine.
  VARIANT 72: note = X is leucine or methionine.
  VARIANT 80: note = X is asparagine, alanine, serine or glutamine.
  VARIANT 165: note = X is alanine or threonine.
  VARIANT 166: note = X is valine or alanine.
  VARIANT 170: note = X is isoleucine, glutamic acid or aspartic acid.
  VARIANT 176: note = X is glutamine or histidine.
  VARIANT 183: note = X is alanine or proline.
  VARIANT 205: note = X is leucine or serine.
  VARIANT 211: note = X is leucine or methionine.
  VARIANT 219: note = X is asparagine, alanine, serine or glutamine.
EDELQVIQPEKSVSVAAGESATLRCXXTSLXPVGPIXWFRGAGXGRELIYNQHEGHFPRVTTVSEXTKRNNXDFSISISXITPADAGTYYCVKFRKGSPDDVEFKSGAGTELSVRAKPSGGGGSGGGGSGGGGSGGGGSEDELQVIQPEKSVSVAAGESATLRCXXTSLXPVGPIXWFRGAGXGRELIYNQHEGHFPRVTTVSEXTKRNNXDFSISISXITPADAGTYYCVKFRKGSPDDVEFKSGAGTELSVRAKPS

SEQ ID NO: 93 - protein, 258 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine or glutamine.
  VARIANT 101: note = X is aspartic acid.
  VARIANT 170: note = X is isoleucine, glutamic acid or aspartic acid.
  VARIANT 176: note = X is glutamine or histidine
  VARIANT 219: note = X is asparagine, alanine, serine or glutamine.
  VARIANT 240: note = X is aspartic acid.
EEELQVIQPDKSISVAAGESATLHCTVTSLXPVGPIXWFRGAGPGRELIYNQHEGHFPRVTTVSDLTKRNNMDFSIRISXITPADAGTYYCVKFRKGSPDXVEFKSGAGTELSVRAKPSGGGGSGGGGSGGGGSGGGGSEEELQVIQPDKSISVAAGESATLHCTVTSLXPVGPIXWFRGAGPGRELIYNQHEGHFPRVTTVSDLTKRNNMDFSIRISXITPADAGTYYCVKFRKGSPDXVEFKSGAGTELSVRAKPS

SEQ ID NO: 94 - protein, 258 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine or glutamine.
  VARIANT 101: note = X is aspartic acid.
  VARIANT 170: note = X is isoleucine, glutamic acid or aspartic acid.
  VARIANT 176: note = X is glutamine or histidine.
  VARIANT 219: note = X is asparagine, alanine, serine or glutamine.
  VARIANT 240: note = X is aspartic acid.
EEELQVIQPDKSISVAAGESATLHCTVTSLXPVGPIXWFRGAGPGRELIYNHKEGHFPRVTTVSDLTKRNNMDFSIRISXITPADAGTYYCVKFRKGSPDXVEFKSGAGTELSVRAKPSGGGGSGGGGSGGGGSGGGGSEEELQVIQPDKSISVAAGESATLHCTVTSLXPVGPIXWFRGAGPGRELIYNHKEGHFPRVTTVSDLTKRNNMDFSIRISXITPADAGTYYCVKFRKGSPDXVEFKSGAGTELSVRAKPS

SEQ ID NO: 95 - protein, 258 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine or glutamine.
  VARIANT 101: note = X is aspartic acid.
  VARIANT 170: note = X is isoleucine, glutamic acid or aspartic acid.
  VARIANT 176: note = X is glutamine or histidine.
  VARIANT 219: note = X is asparagine, alanine, serine or glutamine.
  VARIANT 240: note = X is aspartic acid.
EEELQVIQPDKSISVAAGESATLHCTVTSLXPVGPIXWFRGAGPGRELIYNQKEGHFPRVTTVSDLTHRNNMDFSIRISXITPADAGTYYCVKFRKGSPDXVEFKSGAGTELSVRAKPSGGGGSGGGGSGGGGSGGGGSEEELQVIQPDKSISVAAGESATLHCTVTSLXPVGPIXWFRGAGPGRELIYNQKEGHFPRVTTVSDLTHRNNMDFSIRISXITPADAGTYYCVKFRKGSPDXVEFKSGAGTELSVRAKPS

SEQ ID NO: 96 - protein, 258 aa, synthetic construct
  VARIANT 31: note = X is isoleucine, glutamic acid or aspartic acid.
  VARIANT 37: note = X is leucine, glutamine or histidine.
  VARIANT 101: note = B is asparagine or aspartic acid.
  VARIANT 170: note = X is leucine, glutamic acid or aspartic acid.
  VARIANT 176: note = X is leucine, glutamine or histidine.
  VARIANT 240: note = B is asparagine or aspartic acid.
EEELQMIQPEKLLLVTVGKTATLHCTVTSLXPVGPVXWFRGVGPGRELIYNQHEGHFPRVTTVSDLTKRNNMDFSIRISSITPADVGTYYCVKFRKGSPEBVEFKSGPGTEMALGAKPSGGGGSGGGGSGGGGSGGGGSEEELQMIQPEKLLLVTVGKTATLHCTVTSLXPVGPVXWFRGVGPGRELIYNQHEGHFPRVTTVSDLTKRNNMDFSIRISSITPADVGTYYCVKFRKGSPEBVEFKSGPGTEMALGAKPS

SEQ ID NO: 97 - protein, 394 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
  VARIANT 307: note = X is isoleucine, glutamic acid or aspartic acid.
  VARIANT 313: note = X is glutamine or histidine.
  VARIANT 356: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPS

SEQ ID NO: 98 - protein, 394 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
  VARIANT 307: note = X is isoleucine, glutamic acid or aspartic acid.
  VARIANT 313: note = X is glutamine or histidine.
  VARIANT 356: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPS

SEQ ID NO: 99 - protein, 394 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
  VARIANT 307: note = X is isoleucine, glutamic acid or aspartic acid.
  VARIANT 313: note = X is glutamine or histidine.
  VARIANT 356: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPS

SEQ ID NO: 100 - protein, 394 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
  VARIANT 307: note = X is isoleucine, glutamic acid or aspartic acid.
  VARIANT 313: note = X is glutamine or histidine.
  VARIANT 356: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPS

SEQ ID NO: 101 - protein, 394 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
  VARIANT 307: note = X is isoleucine, glutamic acid or aspartic acid.
  VARIANT 313: note = X is glutamine or histidine.
  VARIANT 356: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPS

SEQ ID NO: 102 - protein, 394 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
  VARIANT 307: note = X is isoleucine, glutamic acid or aspartic acid.
  VARIANT 313: note = X is glutamine or histidine.
  VARIANT 356: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPS

SEQ ID NO: 103 - protein, 397 aa, synthetic construct
  VARIANT 26: note = X is alanine or threonine.
  VARIANT 27: note = X is valine or alanine.
  VARIANT 31: note = X is isoleucine, glutamic acid or aspartic acid.
  VARIANT 37: note = X is glutamine or histidine.
  VARIANT 44: note = X is alanine or proline.
  VARIANT 66: note = X is leucine or serine.
  VARIANT 72: note = X is leucine or methionine.
  VARIANT 80: note = X is asparagine, alanine, serine or glutamine.
  VARIANT 165: note = X is alanine or threonine.
  VARIANT 166: note = X is valine or alanine.
  VARIANT 170: note = X is isoleucine, glutamic acid or aspartic acid.
  VARIANT 176: note = X is glutamine or histidine.
  VARIANT 183: note = X is alanine or proline.
  VARIANT 205: note = X is leucine or serine.
  VARIANT 211: note = X is leucine or methionine.
  VARIANT 219: note = X is asparagine, alanine, serine or glutamine.
  VARIANT 304: note = X is alanine or threonine.
  VARIANT 305: note = X is valine or alanine.
  VARIANT 309: note = X is isoleucine, glutamic acid or aspartic acid.
  VARIANT 315: note = X is glutamine or histidine.
  VARIANT 322: note = X is alanine or proline.
  VARIANT 344: note = X is leucine or serine.
  VARIANT 350: note = X is leucine or methionine.
  VARIANT 358: note = X is asparagine, alanine, serine or glutamine.
EDELQVIQPEKSVSVAAGESATLRCXXTSLXPVGPIXWFRGAGXGRELIYNQHEGHFPRVTTVSEXTKRNNXDFSISISXITPADAGTYYCVKFRKGSPDDVEFKSGAGTELSVRAKPSGGGGSGGGGSGGGGSGGGGSEDELQVIQPEKSVSVAAGESATLRCXXTSLXPVGPIXWFRGAGXGRELIYNQHEGHFPRVTTVSEXTKRNNXDFSISISXITPADAGTYYCVKFRKGSPDDVEFKSGAGTELSVRAKPSGGGGSGGGGSGGGGSGGGGSEDELQVIQPEKSVSVAAGESATLRCXXTSLXPVGPIXWFRGAGXGRELIYNQHEGHFPRVTTVSEXTKRNNXDFSISISXITPADAGTYYCVKFRKGSPDDVEFKSGAGTELSVRAKPS

SEQ ID NO: 104 - protein, 397 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine or glutamine.
  VARIANT 101: note = X is aspartic acid.
  VARIANT 170: note = X is isoleucine, glutamic acid or aspartic acid.
  VARIANT 176: note = X is glutamine or histidine.
  VARIANT 219: note = X is asparagine, alanine, serine or glutamine.
  VARIANT 240: note = X is aspartic acid.
  VARIANT 309: note = X is isoleucine, glutamic acid or aspartic acid.
  VARIANT 315: note = X is glutamine or histidine.
  VARIANT 358: note = X is asparagine, alanine, serine or glutamine.
  VARIANT 379: note = X is aspartic acid.
EEELQVIQPDKSISVAAGESATLHCTVTSLXPVGPIXWFRGAGPGRELIYNQHEGHFPRVTTVSDLTKRNNMDFSIRISXITPADAGTYYCVKFRKGSPDXVEFKSGAGTELSVRAKPSGGGGSGGGGSGGGGSGGGGSEEELQVIQPDKSISVAAGESATLHCTVTSLXPVGPIXWFRGAGPGRELIYNQHEGHFPRVTTVSDLTKRNNMDFSIRISXITPADAGTYYCVKFRKGSPDXVEFKSGAGTELSVRAKPSGGGGSGGGGSGGGGSGGGGSEEELQVIQPDKSISVAAGESATLHCTVTSLXPVGPIXWFRGAGPGRELIYNQHEGHFPRVTTVSDLTKRNNMDFSIRISXITPADAGTYYCVKFRKGSPDXVEFKSGAGTELSVRAKPS

SEQ ID NO: 105 - protein, 397 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine or glutamine.
  VARIANT 101: note = X is aspartic acid.
  VARIANT 170: note = X is isoleucine, glutamic acid or aspartic acid.
  VARIANT 176: note = X is glutamine or histidine.
  VARIANT 219: note = X is asparagine, alanine, serine or glutamine.
  VARIANT 240: note = X is aspartic acid.
  VARIANT 309: note = X is isoleucine, glutamic acid or aspartic acid.
  VARIANT 315: note = X is glutamine or histidine.
  VARIANT 358: note = X is asparagine, alanine, serine or glutamine.
  VARIANT 379: note = X is aspartic acid.
EEELQVIQPDKSISVAAGESATLHCTVTSLXPVGPIXWFRGAGPGRELIYNHKEGHFPRVTTVSDLTKRNNMDFSIRISXITPADAGTYYCVKFRKGSPDXVEFKSGAGTELSVRAKPSGGGGSGGGGSGGGGSGGGGSEEELQVIQPDKSISVAAGESATLHCTVTSLXPVGPIXWFRGAGPGRELIYNHKEGHFPRVTTVSDLTKRNNMDFSIRISXITPADAGTYYCVKFRKGSPDXVEFKSGAGTELSVRAKPSGGGGSGGGGSGGGGSGGGGSEEELQVIQPDKSISVAAGESATLHCTVTSLXPVGPIXWFRGAGPGRELIYNQHEGHFPRVTTVSDLTKRNNMDFSIRISXITPADAGTYYCVKFRKGSPDXVEFKSGAGTELSVRAKPS

SEQ ID NO: 106 - protein, 397 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine or glutamine.
  VARIANT 101: note = X is aspartic acid.
  VARIANT 170: note = X is isoleucine, glutamic acid or aspartic acid.
  VARIANT 176: note = X is glutamine or histidine.
  VARIANT 219: note = X is asparagine, alanine, serine or glutamine.
  VARIANT 240: note = X is aspartic acid.
  VARIANT 309: note = X is isoleucine, glutamic acid or aspartic acid.
  VARIANT 315: note = X is glutamine or histidine.
  VARIANT 358: note = X is asparagine, alanine, serine or glutamine.
  VARIANT 379: note = X is aspartic acid.
EEELQVIQPDKSISVAAGESATLHCTVTSLXPVGPIXWFRGAGPGRELIYNQKEGHFPRVTTVSDLTHRNNMDFSIRISXITPADAGTYYCVKFRKGSPDXVEFKSGAGTELSVRAKPSGGGGSGGGGSGGGGSGGGGSEEELQVIQPDKSISVAAGESATLHCTVTSLXPVGPIXWFRGAGPGRELIYNQKEGHFPRVTTVSDLTHRNNMDFSIRISXITPADAGTYYCVKFRKGSPDXVEFKSGAGTELSVRAKPSGGGGSGGGGSGGGGSGGGGSEEELQVIQPDKSISVAAGESATLHCTVTSLXPVGPIXWFRGAGPGRELIYNQHEGHFPRVTTVSDLTKRNNMDFSIRISXITPADAGTYYCVKFRKGSPDXVEFKSGAGTELSVRAKPS

SEQ ID NO: 107 - protein, 397 aa, synthetic construct
  VARIANT 31: note = X is isoleucine, glutamic acid or aspartic acid.
  VARIANT 37: note = X is leucine, glutamine or histidine.
  VARIANT 101: note = B is asparagine or aspartic acid.
  VARIANT 170: note = X is leucine, glutamic acid or aspartic acid.
  VARIANT 176: note = X is leucine, glutamine or histidine.
  VARIANT 240: note = B is asparagine or aspartic acid.
  VARIANT 309: note = X is leucine, glutamic acid or aspartic acid..
  VARIANT 315: note = X is leucine, glutamine or histidine.
  VARIANT 379: note = B is asparagine or aspartic acid.
EEELQMIQPEKLLLVTVGKTATLHCTVTSLXPVGPVXWFRGVGPGRELIYNQHEGHFPRVTTVSDLTKRNNMDFSIRISSITPADVGTYYCVKFRKGSPEBVEFKSGPGTEMALGAKPSGGGGSGGGGSGGGGSGGGGSEEELQMIQPEKLLLVTVGKTATLHCTVTSLXPVGPVXWFRGVGPGRELIYNQHEGHFPRVTTVSDLTKRNNMDFSIRISSITPADVGTYYCVKFRKGSPEBVEFKSGPGTEMALGAKPSGGGGSGGGGSGGGGSGGGGSEEELQMIQPEKLLLVTVGKTATLHCTVTSLXPVGPVXWFRGVGPGRELIYNQHEGHFPRVTTVSDLTKRNNMDFSIRISSITPADVGTYYCVKFRKGSPEBVEFKSGPGTEMALGAKPS

SEQ ID NO: 108 - protein, 397 aa, synthetic construct
  VARIANT 31: note = X is isoleucine, glutamic acid or aspartic acid.
  VARIANT 37: note = X is leucine, glutamine or histidine.
  VARIANT 101: note = B is asparagine or aspartic acid.
  VARIANT 170: note = X is leucine, glutamic acid or aspartic acid.
  VARIANT 176: note = X is leucine, glutamine or histidine.
  VARIANT 240: note = B is asparagine or aspartic acid.
  VARIANT 309: note = X is leucine, glutamic acid or aspartic acid.
  VARIANT 315: note = X is leucine, glutamine or histidine.
  VARIANT 379: note = B is asparagine or aspartic acid.
EEELQMIQPEKLLLVTVGKTATLHCTVTSLXPVGPVXWFRGVGPGRELIYNQHEGHFPRVTTVSDLTKRNNMDFSIRISSITPADVGTYYCVKFRKGSPEBVEFKSGPGTEMALGAKPSGGGGSGGGGSGGGGSGGGGSEEELQMIQPEKLLLVTVGKTATLHCTVTSLXPVGPVXWFRGVGPGRELIYNQHEGHFPRVTTVSDLTKRNNMDFSIRISSITPADVGTYYCVKFRKGSPEBVEFKSGPGTEMALGAKPSGGGGSGGGGSGGGGSGGGGSEEELQMIQPEKLLLVTVGKTATLHCTVTSLXPVGPVXWFRGVGPGRELIYNQKEGHFPRVTTVSDLTKHNNMDFSIRISSITPADVGTYYCVKFRKGSPEBVEFKSGPGTEMALGAKPS

SEQ ID NO: 109 - protein, 397 aa, synthetic construct
  VARIANT 31: note = X is isoleucine, glutamic acid or aspartic acid.
  VARIANT 37: note = X is leucine, glutamine or histidine.
  VARIANT 101: note = B is asparagine or aspartic acid.
  VARIANT 170: note = X is leucine, glutamic acid or aspartic acid.
  VARIANT 176: note = X is leucine, glutamine or histidine.
  VARIANT 240: note = B is asparagine or aspartic acid.
  VARIANT 309: note = X is leucine, glutamic acid or aspartic acid.
  VARIANT 315: note = X is leucine, glutamine or histidine.
  VARIANT 379: note = B is asparagine or aspartic acid.
EEELQMIQPEKLLLVTVGKTATLHCTVTSLXPVGPVXWFRGVGPGRELIYNQKEGHFPRVTTVSDLTKHNNMDFSIRISSITPADVGTYYCVKFRKGSPEBVEFKSGPGTEMALGAKPSGGGGSGGGGSGGGGSGGGGSEEELQMIQPEKLLLVTVGKTATLHCTVTSLXPVGPVXWFRGVGPGRELIYNQHEGHFPRVTTVSDLTKRNNMDFSIRISSITPADVGTYYCVKFRKGSPEBVEFKSGPGTEMALGAKPSGGGGSGGGGSGGGGSGGGGSEEELQMIQPEKLLLVTVGKTATLHCTVTSLXPVGPVXWFRGVGPGRELIYNQHEGHFPRVTTVSDLTKRNNMDFSIRISSITPADVGTYYCVKFRKGSPEBVEFKSGPGTEMALGAKPS

SEQ ID NO: 110 - protein, 532 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
  VARIANT 307: note = X is isoleucine, glutamic acid or aspartic acid.
  VARIANT 313: note = X is glutamine or histidine.
  VARIANT 356: note = X is asparagine, alanine, serine, or glutamine.
  VARIANT 445: note = X is isoleucine, glutamic acid or aspartic acid.
  VARIANT 451: note = X is glutamine or histidine.
  VARIANT 494: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPS

SEQ ID NO: 111 - protein, 532 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
  VARIANT 307: note = X is isoleucine, glutamic acid or aspartic acid.
  VARIANT 313: note = X is glutamine or histidine.
  VARIANT 356: note = X is asparagine, alanine, serine, or glutamine.
  VARIANT 445: note = X is isoleucine, glutamic acid or aspartic acid.
  VARIANT 451: note = X is glutamine or histidine.
  VARIANT 494: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPS

SEQ ID NO: 112 - protein, 532 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
  VARIANT 307: note = X is isoleucine, glutamic acid or aspartic acid.
  VARIANT 313: note = X is glutamine or histidine.
  VARIANT 356: note = X is asparagine, alanine, serine, or glutamine.
  VARIANT 445: note = X is isoleucine, glutamic acid or aspartic acid.
  VARIANT 451: note = X is glutamine or histidine.
  VARIANT 494: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPS

SEQ ID NO: 113 - protein, 532 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
  VARIANT 307: note = X is isoleucine, glutamic acid or aspartic acid.
  VARIANT 313: note = X is glutamine or histidine.
  VARIANT 356: note = X is asparagine, alanine, serine, or glutamine.
  VARIANT 445: note = X is isoleucine, glutamic acid or aspartic acid.
  VARIANT 451: note = X is glutamine or histidine.
  VARIANT 494: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPS

SEQ ID NO: 114 - protein, 224 aa, Homo sapiens
APVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 115 - protein, 216 aa, Homo sapiens
APVVSGPAVRATPEHTVSFTCESHGFSPRDITLKWFKNGNELSDFQTNVDPAGDSVSYSIHSTARVVLTRGDVHSQVICEIAHITLQGDPLRGTANLSEAIRVPPTLEVTQQPMRAENQANVTCQVSNFYPRGLQLTWLENGNVSRTETASTLIENKDGTYNWMSWLLVNTCAHRDDVVLTCQVEHDGQQAVSKSYALEISAHQKEHGSDITHEAA

SEQ ID NO: 116 - protein, 216 aa, Homo sapiens
APVVSGPAARATPQHTVSFTCESHGFSPRDITLKWFKNGNELSDFQTNVDPAGDSVSYSIHSTAKVVLTREDVHSQVICEVAHVTLQGDPLRGTANLSETIRVPPTLEVTQQPVRAENQVNVTCQVRKFYPQRLQLTWLENGNVSRTETASTLTENKDGTYNWMSWLLVNVSAHRDDVKLTCQVEHDGQPAVSKSHDLKVSAHPKEQGSNTAPGPA

SEQ ID NO: 117 - protein, 220 aa, Homo sapiens
APVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASSLT

SEQ ID NO: 118 - protein, 5 aa, synthetic construct
GGGGS

SEQ ID NO: 119 - protein, 10 aa, synthetic construct
GGGGSGGGGS

SEQ ID NO: 120 - protein, 15 aa, synthetic construct
GGGGSGGGGSGGGGS

SEQ ID NO: 121 - protein, 20 aa, synthetic construct
GGGGSGGGGSGGGGSGGGGS

SEQ ID NO: 122 - protein, 25 aa, synthetic construct
GGGGSGGGGSGGGGSGGGGSGGGGS

SEQ ID NO: 123 - protein, 30 aa, synthetic construct
GGGGSGGGGSGGGGSGGGGSGGGGSGGGGS

SEQ ID NO: 124 - protein, 107 aa, Homo sapiens [sequence omitted]

SEQ ID NO: 125 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 126 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 127 - protein, 101 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERK

SEQ ID NO: 128 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 129 - protein, 5 aa, synthetic construct
EPKSS

SEQ ID NO: 130 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 131 - protein, 225 aa, Homo sapiens
CCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 132 - protein, 227 aa, synthetic construct
  VARIANT 77: note = X is alanine, glycine, serine, or glutamine.
DKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 227 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 138 - protein, 229 aa, synthetic construct
ESKYGPPCPPCPAPEFLGGPSVFLFPPKPKDILMISRIPEVICVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTIPPVLDSDGSFFLYSRLTVDKSRWQEGNVFSCSVMHEALHNHYTQKSLSLSLGK

SEQ ID NO: 139 - protein, 229 aa, synthetic construct
ESKYGPPCPPCPAPEFLGGPSVFLFPPKPKDILMISRIPEVICVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTIPPVLDSDGSFFLVSRLTVDKSRWQEGNVFSCSVMHEALHNHYTQKSLSLSLGK

SEQ ID NO: 140 - protein, 227 aa, synthetic construct
  VARIANT 77: note = X is alanine, glycine, serine, or glutamine.
DKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 227 aa, synthetic construct
  VARIANT 77: note = X is alanine, glycine, serine, or glutamine.
DKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 142 - protein, 227 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 227 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 144 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 145 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 146 - protein, 122 aa, synthetic construct [sequence omitted]

SEQ ID NO: 147 - protein, 107 aa, synthetic construct [sequence omitted]

SEQ ID NO: 148 - protein, 119 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 149 - protein, 107 aa, synthetic construct
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 150 - protein, 121 aa, synthetic construct [sequence omitted]

SEQ ID NO: 151 - protein, 106 aa, synthetic construct [sequence omitted]

SEQ ID NO: 152 - protein, 120 aa, synthetic construct [sequence omitted]

SEQ ID NO: 153 - protein, 107 aa, synthetic construct [sequence omitted]

SEQ ID NO: 154 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 155 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 156 - protein, 114 aa, synthetic construct [sequence omitted]

SEQ ID NO: 157 - protein, 107 aa, synthetic construct [sequence omitted]

SEQ ID NO: 158 - protein, 113 aa, synthetic construct [sequence omitted]

SEQ ID NO: 159 - protein, 113 aa, synthetic construct [sequence omitted]

SEQ ID NO: 160 - protein, 118 aa, synthetic construct
QVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSS

SEQ ID NO: 161 - protein, 113 aa, synthetic construct
DIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIK

SEQ ID NO: 162 - protein, 117 aa, synthetic construct
EVQLQQSGPELVKPGASMKISCKASGYSFTGYTMNWVKQSHGKNLEWIGLINPYNGGTIYNQKFKGKATLTVDKSSSTAYMELLSLTSEDSAVYYCARDYGFVLDYWGQGTTLTVSS

SEQ ID NO: 163 - protein, 107 aa, synthetic construct
QIVLTQSPAIMSASPGEKVTITCSASSSVSYLHWFQQKPGTSPKLWVYSTSNLPSGVPARFGGSGSGTSYSLTISRMEAEDAATYYCQQRSIYPPWTFGGGTKLEIK

SEQ ID NO: 164 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKTNSAKFDPWGQGTMVTVSS

SEQ ID NO: 165 - protein, 111 aa, synthetic construct
QSALTQPASVSGSPGQSITISCTGTSNDVGAYNYVSWYQQHPGKAPKLMISEVNKRPSGVSNRFSGSKSGNTASLTISGLQAEDEADYYCSSYTMGSTFMLFGGGTKLTVL

SEQ ID NO: 166 - protein, 119 aa, synthetic construct
QVQLQESGPELVKPGASVKMSCKASGYSFTAYNMHWVKQSHGKSLEWIGFIDPYSGIITYNQTFKGKATLTVDKSSRTAYMQLNSLTSEDSAVYYCARRGYYDGGFDYWGQGTTLTVSS

SEQ ID NO: 167 - protein, 107 aa, synthetic construct
DIQMTQTTSSLSASLGDRVTISCRASQDITNYLNWYQQKPDGTVKLLIYYTSRLHSGVPSRFSGSGSGTDYSLTISNLEQEDIATYFCQQDSKHPRTFGGGTKLEIK

SEQ ID NO: 168 - protein, 120 aa, synthetic construct
EVQLQQSGPELEKPGASVKLSCKASGYSFTGYNMNWVKQSHGKSLEWIGHIDPYYGDTSYNQKFRGKATLTVDKSSSTAYMQLKSLTSEDSAVYYCVKGGYYGHWYFDVWGAGTTVTVSS

SEQ ID NO: 169 - protein, 107 aa, synthetic construct
DIQMTQSPSSLSASLGERVSLTCRASQDIGSSLNWLQQGPDGTIKRLIYATSSLDSGVPKRFSGSRSGSDYSLTISSLESEDFVDYYCLQYVSSPPTFGAGTKLELK

SEQ ID NO: 170 - protein, 124 aa, synthetic construct
EVQLVESGGGLVQPGGSLRLSCATSGFDFSRYWMSWVRQAPGKGLVWIGEVNPDSTSINYTPSLKDQFTISRDNAKNTLYLQMNSLRAEDTAVYYCTRPNYYGSRYHYYAMDYWGQGTLVTVSS

SEQ ID NO: 171 - protein, 107 aa, synthetic construct
DIQMTQSPSSLSASVGDRVTITCRASQDINNYLNWYQQKPGKAPKLLIYYTSRLHSGVPSRFSGSGSGTDFTFTISSLQPEDIATYYCQQGSTLPFTFGQGTKLEIK

SEQ ID NO: 172 - protein, 117 aa, synthetic construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 173 - protein, 107 aa, synthetic construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 174 - protein, 117 aa, synthetic construct
EVQLLESGGGLVQPGGSLRLSCAASGFTFSNAWMSWVRQAPGKGLEWVAFIWYDGSNKYYADSVKGRFTISRDNSKNTLYLQMNSLRAEDTAVYYCARYSGWYFDYWGQGTLVTVSS

SEQ ID NO: 175 - protein, 111 aa, synthetic construct
QSVLTQPPSASGTPGQRVTISCTGSSSNIGAGYDVHWYQQLPGTAPKLLIYDNNNRPSGVPDRFSGSKSGTSASLAISGLRSEDEADYYCQSYDSSLSAWLFGGGTKLTVL

SEQ ID NO: 176 - protein, 127 aa, synthetic construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSS

SEQ ID NO: 177 - protein, 107 aa, synthetic construct
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 178 - protein, 122 aa, synthetic construct
QVQLQESGPGLVKPSETLSLTCAVSGYSILSGYYWFWIRQPPGKGLEWIGGIYHSGSTAYNPSLKSRVTISVDTSKNQFSLKLSSVTAADTAVYYCARGGEVTYSRGPLDVWGQGTTVTVSS

SEQ ID NO: 179 - protein, 108 aa, synthetic construct
EIVLTQSPGTLSLSPGERATLSCRASNAVSSSYLAWYQQKPGQAPRLLIYGASYRATGIPDRFSGSGSGTDFTLTISRLEPEDFAVYYCQQHSLYPPTFGGGTKVEIK

SEQ ID NO: 180 - protein, 113 aa, synthetic construct
QVQLVESGGGVVQPGRSLRLSCAASGFTFSRYWMSWVRQAPGKGLEWIAEINPDSSTINYTPSLRDKFTISRDNSKNTLYLQMSSLRAEDTAVYYCARQGDYWGQGTLVTVSS

SEQ ID NO: 181 - protein, 112 aa, synthetic construct
DIVMTQTPLSLSVTPGQPASISCKSSQSLLHSDGKTYLNWYLQKPGQSPQRLIYLVSKLDSGVPDRFSGSGSGTDFTLKISRVEAEDVGVYYCWQGAHFPYTFGQGTKLEIK

SEQ ID NO: 182 - protein, 122 aa, synthetic construct
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSS

SEQ ID NO: 183 - protein, 108 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 184 - protein, 118 aa, synthetic construct
QVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVSS

SEQ ID NO: 185 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIK

SEQ ID NO: 186 - protein, 120 aa, synthetic construct
EGQLVQSGGGLVHPGGSLRLSCAGSGFTFSSYGMHWVRQAPGKGLEWVSGIGTGGGTYSTDSVKGRFTISRDNAKNSLYLQMNSLRAEDMAVYYCARGDYYGSGSFFDCWGQGTLVTVSS

SEQ ID NO: 187 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIK

SEQ ID NO: 188 - protein, 116 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSS

SEQ ID NO: 189 - protein, 106 aa, synthetic construct
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIK

SEQ ID NO: 190 - protein, 118 aa, synthetic construct
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSS

SEQ ID NO: 191 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIK

SEQ ID NO: 192 - protein, 121 aa, synthetic construct
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSS

SEQ ID NO: 193 - protein, 106 aa, synthetic construct
QIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIK

SEQ ID NO: 194 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSS

SEQ ID NO: 195 - protein, 107 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAYSFPFTFGPGTKVDIE

SEQ ID NO: 196 - protein, 119 aa, synthetic construct
QSLEESGGRLVTPGTPLTLTCTVSGIDLSSYALGWVRQAPGKGLEYIGIISSTGTTYYATWAKGRFTISKTSSTTVDLKITGTTEDTATCARGAYAGYVAFGPYYFHIWGPGTLVTISL

SEQ ID NO: 197 - protein, 111 aa, synthetic construct
DIVMTQTPASVEAAVGGTVTIKCQASQSISNFLAWYQQKPGQPPKVLIYAASHLASGVPSRFKGSGSGTQFTLTISDLECADAATYYCQSYFYSSTSIYNAFGGGTEVVVR

SEQ ID NO: 198 - protein, 116 aa, synthetic construct
EVQLVQSGAEVKKPGESLRISCKGSGDSFTTYWIGWVRQMPGKGLEWMGIIYPGDSDTIYSPSFQGQVTISADKSISTAYLQWSSLKASDTAMYYCTRGDRGVDYWGQGTLVTVSS

SEQ ID NO: 199 - protein, 107 aa, synthetic construct
DIQMTQSPSSLSASVGDRVTITCRASQGISRWLAWYQQKPEKAPKSLIYAASSLQSGVPSRFSGSGSGTDFTLTISGLQPEDFATYYCQQYNSYPRTFGQGTKVEIK

SEQ ID NO: 200 - protein, 119 aa, synthetic construct
QVQLQQSGSELKKPGASVKISCKASGYSFTSYWMHWVKQRPGQGLEWIGMIDPFDSESRLNQEFKDKATLTVDKPSNTAYMQLSSLTSEDSAVYYCTRRNGGYYVFDSWGQGTTLTVSS

SEQ ID NO: 201 - protein, 112 aa, synthetic construct
DVVMTQSPLSLPVTLGQSASISCRSSQNIVHSNGDTYLEWFLQRPGQSPQLLIFKVSNRFSGVPDRFSGSGSGTDFTLKISRVEAEDVGVYYCFQGSHVPWTFGGGTKLEIK

SEQ ID NO: 202 - protein, 116 aa, synthetic construct
QVQLVQSGAEVKKPGASVKMSCKASGYTFTDYVINWVRQAPGQGLEWIGEIYPGSGSTFYAQKFQGRATLTADTSTSTAYMEVSSLRSEDTAVYYCTRRQAAMDYWGQGTLVTVSS

SEQ ID NO: 203 - protein, 105 aa, synthetic construct
DIQLTQSPSSLSASVGDRVTITCSASSSVSYMHWYQQKPGKAPKLLLYTTSNLASGVPSRFSGSGSGTDYTLTISSLQPEDFATYYCHQWSGYPTFGQGTKLEIK

SEQ ID NO: 204 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 205 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 206 - protein, 125 aa, synthetic construct
EVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 207 - protein, 109 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 208 - protein, 117 aa, synthetic construct
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSS

SEQ ID NO: 209 - protein, 110 aa, synthetic construct
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIK

SEQ ID NO: 210 - protein, 125 aa, synthetic construct
QVQLVQSGAEVKKPGASVKVSCKASGYTFTDYYINWVRQAPGQGLEWMGNVNPNDGGTTYNQKFKGRVTMTTDTSTSTAYMELRSLRSDDTAVYYCARREIYFYGTIYYYAMDYWGQGTLVTVSS

SEQ ID NO: 211 - protein, 109 aa, synthetic construct
DIQLTQSPSFLSASVGDRVTITCRASESVDYYGNSFMYWYQQKPGKAPKWYFASNLESGVPSRFSGSGSGTEFTLTISSLQPEDFATYYCQQNNEDPWTFGGGTKVEIK

SEQ ID NO: 212 - protein, 118 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 213 - protein, 108 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 214 - protein, 115 aa, synthetic construct [sequence omitted]

SEQ ID NO: 215 - protein, 10 aa, synthetic construct
KFRKAFKRFF

SEQ ID NO: 216 - protein, 224 aa, synthetic construct
SGLRINSAKDDAAGQAAANRATSNIKGLTQASRNAADGISIAQTTEGALNEINNNLQRVRELSVQATAGANADALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGTSTLAQ

SEQ ID NO: 217 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 218 - protein, 171 aa, Homo sapiens
GGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCG

SEQ ID NO: 219 - protein, 171 aa, synthetic construct
GGTRSPRCDCAGDFHKKTGLFCCRGCPAGHYLKAPCTEPCGNSTCLPCPQGTFLAWENHHNSECTRCQACDEQASQVALENCSAVADTRCGCAPGWFVECSVSQCVSSSPFYCQPCLDCGALHRHTRLPCSRRDTDCGTCLPGFYEHGDGCVSCPTSTLGSCPERCAAVCG

SEQ ID NO: 220 - protein, 171 aa, synthetic construct
GGTRSPRCDCAGDFHKKTGLFCCRGCPAGHYLKAPCTEPCGNSTCLVCPQGTFLAWENHHNSECARCQACDEQASQVALENCSAVADTRCGCEPGWFVECQVSQCVSSSPFYCQPCLDCGALHRHTRLPCSRRDTDCGTCLPGFYEHGDGCVSCPTSTLGSCPERCAAVCG

SEQ ID NO: 221 - protein, 396 aa, synthetic construct
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRSKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQA

SEQ ID NO: 222 - protein, 257 aa, synthetic construct
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRSKDDEFTHLYTLIVRPDNTYEVKIDNSQVESGSLEDDWDFLPGSGDPSIYAYDNFGVLGLDLWQVKSGTIFDNFLITNDEAYAEEFGNETWGVTKAAEKQMKDKQDEEQRLK

SEQ ID NO: 223 - protein, 351 aa, synthetic construct
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRSKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLK

SEQ ID NO: 224 - protein, 332 aa, Homo sapiens
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSG

SEQ ID NO: 225 - protein, 327 aa, Homo sapiens
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSG

SEQ ID NO: 226 - protein, 530 aa, Homo sapiens
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLIST

SEQ ID NO: 227 - protein, 435 aa, Homo sapiens
GHLPKPTLWAEPGSVITQGSPVTLRCQGGQETQEYRLYREKKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRH

SEQ ID NO: 228 - protein, 435 aa, Homo sapiens
GTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAYNLSSEWSAPSDPLDILITGQIHGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRH

SEQ ID NO: 229 - protein, 219 aa, Homo sapiens
HVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARPGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTGIARHLH

SEQ ID NO: 230 - protein, 135 aa, Homo sapiens
LFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 231 - protein, 285 aa, Homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQ

SEQ ID NO: 232 - protein, 275 aa, Homo sapiens
EPHSLRYNLMVLSQDGSVQSGFLAEGHLDGQPFLRYDRQKRRAKPQGQWAENVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSG

SEQ ID NO: 233 - protein, 187 aa, Homo sapiens
DTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAP

SEQ ID NO: 234 - protein, 189 aa, Homo sapiens
DPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMSS

SEQ ID NO: 235 - protein, 187 aa, Homo sapiens
DAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAP

SEQ ID NO: 236 - protein, 197 aa, Homo sapiens
GGHSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPD

SEQ ID NO: 237 - protein, 182 aa, Homo sapiens
DPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAG

SEQ ID NO: 238 - protein, 189 aa, Homo sapiens
DPHSLCYDITVIPKFRPGPRWCAVQGQVDEKTFLHYDCGNKTVTPVSPLGKKLNVTTAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSS

SEQ ID NO: 239 - protein, 208 aa, Homo sapiens [sequence omitted]

SEQ ID NO: 240 - protein, 224 aa, Homo sapiens
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 241 - protein, 240 aa, Homo sapiens
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWS

SEQ ID NO: 242 - protein, 161 aa, Homo sapiens
RVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 243 - protein, 161 aa, synthetic construct
RVAAHITGTRGRSNTLSSPNSKNEKALGRKINSWESSRSGHSFLSNLHLRNGELVIHEKGFYYIYSQTAFRFSEEIKEVTRNDKQMVQYIYKWTDYPDPILLMKSARNSCWSKDAEYGLYSIYQGGIFELKENDRIFVSVTNEHLIDMDHEASFFGAFLVG

SEQ ID NO: 244 - protein, 161 aa, synthetic construct
RVAAHITGTRGRSNTLSSPNSKNEKALGRKINSWESSRSGHSFLSNLHLRNGELVIHEKGFYYIYSQTQFKFREEIKENTKNDKQMVQYIYKYTSYPDPILLMKSARNSCWSKDAEYGLYSIYQGGIFELKENDRIFVSVTNERLLQMDHEASFFGAFLVG

SEQ ID NO: 245 - protein, 148 aa, Homo sapiens
PSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 246 - protein, 157 aa, Homo sapiens [sequence omitted]

SEQ ID NO: 247 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 248 - protein, 133 aa, Homo sapiens
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 249 - protein, 215 aa, Homo sapiens
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 250 - protein, 128 aa, Homo sapiens
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKDNTYWGIILLANPQFIS

SEQ ID NO: 251 - protein, 155 aa, Homo sapiens
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 252 - protein, 182 aa, Homo sapiens
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 253 - protein, 180 aa, Homo sapiens
LKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 254 - protein, 338 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 255 - protein, 338 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 256 - protein, 476 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 257 - protein, 476 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 258 - protein, 476 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 259 - protein, 476 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 260 - protein, 341 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 261 - protein, 341 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 262 - protein, 479 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 263 - protein, 479 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 264 - protein, 479 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 265 - protein, 479 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 266 - protein, 603 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSDIQMTQSPSSLSASVGDRVTITCRASQDISKYLNWYQQKPGKAPKLLIYHTSRLHSGVPSRFSGSGSGTDFTLTISSLQPEDFATYYCQQGNTLPYTFGQGTKVEIKGGGGSGGGGSGGGGSEVQLVESGGGLVQPGGSLRLSCAASGVSLPDYGVSWVRQAPGKGLEWVSVIWGSETTYYNSALKSRFTISRDNAKNSLYLQMNSLRAEDTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 267 - protein, 741 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSDIQMTQSPSSLSASVGDRVTITCRASQDISKYLNWYQQKPGKAPKLLIYHTSRLHSGVPSRFSGSGSGTDFTLTISSLQPEDFATYYCQQGNTLPYTFGQGTKVEIKGGGGSGGGGSGGGGSEVQLVESGGGLVQPGGSLRLSCAASGVSLPDYGVSWVRQAPGKGLEWVSVIWGSETTYYNSALKSRFTISRDNAKNSLYLQMNSLRAEDTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 268 - protein, 323 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 269 - protein, 323 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 270 - protein, 461 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 271 - protein, 461 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 272 - protein, 461 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 273 - protein, 461 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQKEGHFPRVTTVSESTKHENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 274 - protein, 575 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSDIQMTQSPSSLSASVGDRVTITCRASQDISKYLNWYQQKPGKAPKLLIYHTSRLHSGVPSRFSGSGSGTDFTLTISSLQPEDFATYYCQQGNTLPYTFGQGTKVEIKGGGGSGGGGSGGGGSEVQLVESGGGLVQPGGSLRLSCAASGVSLPDYGVSWVRQAPGKGLEWVSVIWGSETTYYNSALKSRFTISRDNAKNSLYLQMNSLRAEDTAVYYCAKHYYYGGSYAMDYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 275 - protein, 713 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSDIQMTQSPSSLSASVGDRVTITCRASQDISKYLNWYQQKPGKAPKLLIYHTSRLHSGVPSRFSGSGSGTDFTLTISSLQPEDFATYYCQQGNTLPYTFGQGTKVEIKGGGGSGGGGSGGGGSEVQLVESGGGLVQPGGSLRLSCAASGVSLPDYGVSWVRQAPGKGLEWVSVIWGSETTYYNSALKSRFTISRDNAKNSLYLQMNSLRAEDTAVYYCAKHYYYGGSYAMDYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 276 - protein, 291 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 277 - protein, 271 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 278 - protein, 429 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 279 - protein, 409 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 280 - protein, 477 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNHKEGHFPRVTTVSESTKRENMDFSISISXITPADAGTYYCVKFRKGSPDTEFKSGAGTELSVRAKPSGGGGSGGGGSGGGGSGGGGSGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 281 - protein, 477 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
EEELQVIQPDKSVSVAAGESAILHCTVTSLXPVGPIXWFRGAGPARELIYNQKEGHFPRVTTVSESTHRENMDFSISISXITPADAGTYYCVKFRKGSPDTEFKSGAGTELSVRAKPSGGGGSGGGGSGGGGSGGGGSGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 282 - protein, 615 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 283 - protein, 753 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
  VARIANT 307: note = X is isoleucine, glutamic acid or aspartic acid.
  VARIANT 313: note = X is glutamine or histidine.
  VARIANT 356: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 284 - protein, 891 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
  VARIANT 307: note = X is isoleucine, glutamic acid or aspartic acid.
  VARIANT 313: note = X is glutamine or histidine.
  VARIANT 356: note = X is asparagine, alanine, serine, or glutamine.
  VARIANT 445: note = X is isoleucine, glutamic acid or aspartic acid.
  VARIANT 451: note = X is glutamine or histidine.
  VARIANT 494: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 285 - protein, 869 aa, synthetic construct
  VARIANT 31: note = X is isoleucine, glutamic acid or aspartic acid.
  VARIANT 37: note = X is glutamine or histidine.
  VARIANT 80: note = X is asparagine, alanine, serine, or glutamine.
  VARIANT 169: note = X is isoleucine, glutamic acid or aspartic acid.
  VARIANT 175: note = X is glutamine or histidine.
  VARIANT 218: note = X is asparagine, alanine, serine, or glutamine.
EEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSGGGGSGGGGSEEELQVIQPDKSVSVAAGESAILHCTVTSLXPVGPIXWFRGAGPARELIYNQHEGHFPRVTTVSESTKRENMDFSISISXITPADAGTYYCVKFRKGSPDTEFKSGAGTELSVRAKPSGGGGSGGGGSDIQMTQSPSSLSASVGDRVTITCRASQDISKYLNWYQQKPGKAPKLLIYHTSRLHSGVPSRFSGSGSGTDFTLTISSLQPEDFATYYCQQGNTLPYTFGQGTKVEIKGGGGSGGGGSGGGGSEVQLVESGGGLVQPGGSLRLSCAASGVSLPDYGVSWVRQAPGKGLEWVSVIWGSETTYYNSALKSRFTISRDNAKNSLYLQMNSLRAEDTAVYYCAKHYYYGGSYAMDYWGQGTLVTVSSGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 286 - protein, 465 aa, synthetic construct
DIQMTQSPSSLSASVGDRVTITCRASQDISKYLNWYQQKPGKAPKLLIYHTSRLHSGVPSRFSGSGSGTDFTLTISSLQPEDFATYYCQQGNTLPYTFGQGTKVEIKGGGGSGGGGSGGGGSEVQLVESGGGLVQPGGSLRLSCAASGVSLPDYGVSWVRQAPGKGLEWVSVIWGSETTYYNSALKSRFTISRDNAKNSLYLQMNSLRAEDTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 287 - DNA, 390 bp, synthetic construct
  source 1..390: mol_type = other DNA
tcgaggtcgacggtatcgataagcttgatatcgaattaggaggaaaaactgtttcatacagaaggcgttaattaggaggaaaaactgtttcatacagaaggcgttaattaggaggaaaaactgtttcatacagaaggcgttaattggtcccatcgaattaggaggaaaaactgtttcatacagaaggcgttaattaggaggaaaaactgtttcatacagaaggcgttaattaggaggaaaaactgtttcatacagaaggcgttaattggtcccgggacattttgacacccccataatatttttccagaattaacagtataaattgcatctcttgttcaagagttccctatcactctctttaatcactactcacagtaacctcaactcctg

SEQ ID NO: 288 - protein, 621 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSGIIQHDLIFSLQQTECVLKPVESSDMKMTQLFTKVESEDTSSLFDKLKKEPDALTLLAPAAGDTIISLDFGSND

SEQ ID NO: 289 - protein, 400 aa, synthetic construct
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQMGSGATNFSLLKQAGDVEENPGPMKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 290 - DNA, 1602 bp, synthetic construct
  source 1..1602: mol_type = other DNA
tcgaggtcgacggtatcgataagcttgatatcgaattaggaggaaaaactgtttcatacagaaggcgttaattaggaggaaaaactgtttcatacagaaggcgttaattaggaggaaaaactgtttcatacagaaggcgttaattggtcccatcgaattaggaggaaaaactgtttcatacagaaggcgttaattaggaggaaaaactgtttcatacagaaggcgttaattaggaggaaaaactgtttcatacagaaggcgttaattggtcccgggacattttgacacccccataatatttttccagaattaacagtataaattgcatctcttgttcaagagttccctatcactctctttaatcactactcacagtaacctcaactcctggccgccaccatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgggaagcggagccacgaacttctctctgttaaagcaagcaggagacgtggaagaaaaccccggtcctatgaagtataccagctacatcctggccttccagctgtgtattgtgctgggcagcctgggctgctactgtcaggacccctacgtgaaggaggccgagaacctgaagaagtacttcaatgccggccatagcgacgtggccgacaatggcaccctgttcctgggcattctgaagaactggaaggaggagagcgaccggaagatcatgcagtcccagattgtgtccttctactttaagctgttcaaaaacttcaaggacgaccagtctatccagaaatccgtggagaccatcaaggaagacatgaatgtgaaatttttcaattccaacaagaagaagagggatgatttcgagaagctgacaaattactccgtcaccgatctgaatgtgcagaggaaggccatccacgaactgatccaggtgatggccgagctgagccccgccgccaagaccgggaagcggaagcggtctcagatgctgttcaggggccgcagggccagccagtaa

SEQ ID NO: 291 - protein, 453 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 292 - protein, 453 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 293 - protein, 230 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 294 - protein, 230 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 368 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 296 - protein, 368 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 297 - protein, 368 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 298 - protein, 368 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 330 aa, Homo sapiens [sequence omitted]

SEQ ID NO: 300 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - protein, 345 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 302 - protein, 345 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 303 - protein, 345 aa, synthetic construct
EEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 304 - protein, 345 aa, synthetic construct
EEELQVIQPDKSVSVAAGESAILHCTVTSLIPVGPIQWFRGAGPARELIYNQKEGHFPRVTTVSESTHRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 305 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRHGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 345 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 307 - protein, 345 aa, synthetic construct
EEELQVIQPDKSVSVAAGESAILHCTVTSLEPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 346 aa, synthetic construct
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - protein, 346 aa, synthetic construct
EEELQVIQPDKSVLVAAGETATLRCTATSLIPVGPIQWFRGAGPGRELIYNQKEGHFPRVTTVSDLTKHNNMDFSIRIGNITPADAGTYYCVKFRKGSPDDVEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 346 aa, synthetic construct
EEELQVIQPDKSVLVAAGETATLRCTATSLIPVGPIQWFRGAGPGRELIYNQHEGHFPRVTTVSDLTKRNNMDFSIRIGNITPADAGTYYCVKFRKGSPDDVEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 346 aa, synthetic construct
EEELQVIQPDKSVLVAAGETATLRCTATSLIPVGPIQWFRGAGPGRELIYNQKEGHFPRVTTVSDLTHRNNMDFSIRIGNITPADAGTYYCVKFRKGSPDDVEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 312 - protein, 346 aa, synthetic construct
EEELQVIQPDKSVLVAAGETATLRCTATSLIPVGPIQWFRGAGPGRELIYNHKEGHFPRVTTVSDLTKRNNMDFSIRIGNITPADAGTYYCVKFRKGSPDDVEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 345 aa, synthetic construct
EEELQVIQPDKSVLVAAGETATLRCTATSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 345 aa, synthetic construct
EEELQVIQPDKSVLVAAGETATLRCTATSLIPVGPIQWFRGAGPARELIYNQHEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 315 - protein, 345 aa, synthetic construct
EEELQVIQPDKSVLVAAGETATLRCTATSLIPVGPIQWFRGAGPARELIYNQKEGHFPRVTTVSESTKHENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 345 aa, synthetic construct
EEELQVIQPDKSVLVAAGETATLRCTATSLIPVGPIQWFRGAGPARELIYNHKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 317 - protein, 345 aa, synthetic construct
EEELQVIQPDKSVLVAAGETATLRCTATSLIPVGPIQWFRGAGPARELIYNQKEGHFPRVTTVSESTHRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - protein, 346 aa, synthetic construct
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 319 - protein, 346 aa, synthetic construct
EEELQMIQPEKLLLVTVGKTATLHCTVTSLLPVGPVLWFRGVGPGRELIYNQHEGHFPRVTTVSDLTKRNNMDFSIRISSITPADVGTYYCVKFRKGSPENVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 320 - protein, 346 aa, synthetic construct
EEELQMIQPEKLLLVTVGKTATLHCTVTSLLPVGPVLWFRGVGPGRELIYNQKEGHFPRVTTVSDLTKHNNMDFSIRISSITPADVGTYYCVKFRKGSPENVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 346 aa, synthetic construct
EEELQMIQPEKLLLVTVGKTATLHCTVTSLLPVGPVLWFRGVGPGRELIYNQKEGHFPRVTTVSDLTHRNNMDFSIRISSITPADVGTYYCVKFRKGSPENVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 346 aa, synthetic construct
EEELQMIQPEKLLLVTVGKTATLHCTVTSLLPVGPVLWFRGVGPGRELIYNHKEGHFPRVTTVSDLTKRNNMDFSIRISSITPADVGTYYCVKFRKGSPENVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 346 aa, synthetic construct
EEELQMIQPEKLLLVTVGKTATLHCTVTSLLPVGPVQWFRGVGPGRELIYNQHEGHFPRVTTVSDLTKRNNMDFSIRISSITPADVGTYYCVKFRKGSPEDVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 346 aa, synthetic construct
EEELQMIQPEKLLLVTVGKTATLHCTVTSLLPVGPVQWFRGVGPGRELIYNQKEGHFPRVTTVSDLTKHNNMDFSIRISSITPADVGTYYCVKFRKGSPEDVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 346 aa, synthetic construct
EEELQVIQPDKSISVAAGESATLHCTVTSLIPVGPIQWFRGAGPGRELIYNQKEGHFPRVTTVSDLTKRNNMDFSIRISNITPADAGTYYCVKFRKGSPDDVEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 346 aa, synthetic construct
EEELQVIQPDKSISVAAGESATLHCTVTSLIPVGPIQWFRGAGPGRELIYNQHEGHFPRVTTVSDLTKRNNMDFSIRISNITPADAGTYYCVKFRKGSPDDVEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 346 aa, synthetic construct
EEELQVIQPDKSISVAAGESATLHCTVTSLIPVGPIQWFRGAGPGRELIYNQKEGHFPRVTTVSDLTKHNNMDFSIRISNITPADAGTYYCVKFRKGSPDDVEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 346 aa, synthetic construct
EEELQVIQPDKSISVAAGESATLHCTVTSLIPVGPIQWFRGAGPGRELIYNQKEGHFPRVTTVSDLTHRNNMDFSIRISNITPADAGTYYCVKFRKGSPDDVEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 329 - protein, 346 aa, synthetic construct
EEELQVIQPDKSISVAAGESATLHCTVTSLIPVGPIQWFRGAGPGRELIYNHKEGHFPRVTTVSDLTKRNNMDFSIRISNITPADAGTYYCVKFRKGSPDDVEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - protein, 345 aa, synthetic construct
EEELQVIQPDKSVSVAAGESAILHCTVTSLIPVGPIQWFRGAGPARELIYNHKEGHFPRVTTVSESTHRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 331 - protein, 345 aa, synthetic construct
EEELQVIQPDKSVSVAAGESAILHCTVTSLIPVGPIHWFRGAGPARELIYNQHEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 332 - protein, 345 aa, synthetic construct
EEELQVIQPDKSVSVAAGESAILHCTVTSLIPVGPIQWFRGAGPARELIYNHHEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 333 - protein, 345 aa, synthetic construct
EEELQVIQPDKSVSVAAGESAILHCTVTSLIPVGPIQWFRGAGPARELIYNQHEGHFPRVTTVSESTHRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 334 - protein, 345 aa, synthetic construct
EEELQVIQPDKSVSVAAGESAILHCTVTSLIPVGPIHWFRGAGPARELIYNQKEGHFPRVTTVSESTKHENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 345 aa, synthetic construct
EEELQVIQPDKSVSVAAGESAILHCTVTSLIPVGPIQWFRGAGPARELIYNHKEGHFPRVTTVSESTKHENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 345 aa, synthetic construct
EEELQVIQPDKSVSVAAGESAILHCTVTSLIPVGPIQWFRGAGPARELIYNQKEGHFPRVTTVSESTHHENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 337 - protein, 345 aa, synthetic construct
EEELQVIQPDKSVSVAAGESAILHCTVTSLIPVGPIQWFRGAGPARELIYNQHEGHFPRVTTVSESTKHENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 338 - protein, 345 aa, synthetic construct
EEELQVIQPDKSVSVAAGESAILHCTVTSLHPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 339 - protein, 345 aa, synthetic construct
EEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 340 - protein, 345 aa, synthetic construct
EEELQVIQPDKSVSVAAGESAILHCTVTSLIPVGHIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 341 - protein, 345 aa, synthetic construct
EEELQVIQPDKSVSVAAGESAILHCTVTSLIPVGPIHWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 342 - protein, 345 aa, synthetic construct
EEELQVIQPDKSVSVAAGESAILHCTVTSLIPVGPIQWFRGAGPARELIYHQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 343 - protein, 345 aa, synthetic construct
EEELQVIQPDKSVSVAAGESAILHCTVTSLIPVGPIQWFRGAGPARELIYNQKEGHFPRVTTVSEHTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 344 - protein, 345 aa, synthetic construct
EEELQVIQPDKSVSVAAGESAILHCTVTSLIPVGPIQWFRGAGPARELIYNQKEGHFPRVTTVSESTKRHNMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 345 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKHSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 346 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGHPDTEFKHGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 347 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSHDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 345 aa, synthetic construct
EEELQVIQPDKSVSVAAGESAILHCTVTSLIPVGPIQWFHGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 345 aa, synthetic construct
EEELQVIQPDKSVSVAAGESAILHCTVTSLIPVGPIQWFRGAGPAHELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - protein, 345 aa, synthetic construct
EEELQVIQPDKSVSVAAGESAILHCTVTSLIPVGPIQWFRGAGPARELIYNQKEGHFPHVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFHSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 345 aa, synthetic construct
EEELQVIQPDKSVSVAAGESAILHCTVTSLYPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 345 aa, synthetic construct
EEELQVIQPDKSVSVAAGESAILHCTVTSLWPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRRGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 355 - protein, 346 aa, synthetic construct
EEELQMIQPEKLLLVTVGKTATLHCTVTSLLPVGPVQWFRGVGPGRELIYNQKEGHFPRVTTVSDLTKRNNMDFSIRISSITPADVGTYYCVKFRKGSPENVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 356 - protein, 346 aa, synthetic construct
EEELQMIQPEKLLLVTVGKTATLHCTVTSLLPVGPVQWFRGVGPGRELIYNQHEGHFPRVTTVSDLTKRNNMDFSIRISSITPADVGTYYCVKFRKGSPENVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 357 - protein, 346 aa, synthetic construct
EEELQMIQPEKLLLVTVGKTATLHCTVTSLLPVGPVQWFRGVGPGRELIYNQKEGHFPRVTTVSDLTKHNNMDFSIRISSITPADVGTYYCVKFRKGSPENVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 358 - protein, 346 aa, synthetic construct
EEELQMIQPEKLLLVTVGKTATLHCTVTSLLPVGPVQWFRGVGPGRELIYNHKEGHFPRVTTVSDLTKRNNMDFSIRISSITPADVGTYYCVKFRKGSPENVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 359 - protein, 346 aa, synthetic construct
EEELQMIQPEKLLLVTVGKTATLHCTVTSLLPVGPVQWFRGVGPGRELIYNQKEGHFPRVTTVSDLTHRNNMDFSIRISSITPADVGTYYCVKFRKGSPENVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 346 aa, synthetic construct
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPEDVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 361 - protein, 346 aa, synthetic construct
EEELQMIQPEKLLLVTVGKTATLHCTVTSLLPVGPVLWFRGVGPGRELIYNQHEGHFPRVTTVSDLTKRNNMDFSIRISSITPADVGTYYCVKFRKGSPEDVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 346 aa, synthetic construct
EEELQMIQPEKLLLVTVGKTATLHCTVTSLLPVGPVLWFRGVGPGRELIYNQKEGHFPRVTTVSDLTKHNNMDFSIRISSITPADVGTYYCVKFRKGSPEDVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 363 - protein, 346 aa, synthetic construct
EEELQMIQPEKLLLVTVGKTATLHCTVTSLLPVGPVLWFRGVGPGRELIYNHKEGHFPRVTTVSDLTKRNNMDFSIRISSITPADVGTYYCVKFRKGSPEDVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 364 - protein, 346 aa, synthetic construct
EEELQMIQPEKLLLVTVGKTATLHCTVTSLLPVGPVLWFRGVGPGRELIYNQKEGHFPRVTTVSDLTHRNNMDFSIRISSITPADVGTYYCVKFRKGSPEDVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 365 - protein, 346 aa, synthetic construct
EEELQMIQPEKLLLVTVGKTATLHCTVTSLLPVGPVQWFRGVGPGRELIYNQKEGHFPRVTTVSDLTKRNNMDFSIRISSITPADVGTYYCVKFRKGSPEDVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 366 - protein, 346 aa, synthetic construct
EEELQMIQPEKLLLVTVGKTATLHCTVTSLLPVGPVQWFRGVGPGRELIYNHKEGHFPRVTTVSDLTKRNNMDFSIRISSITPADVGTYYCVKFRKGSPEDVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 367 - protein, 346 aa, synthetic construct
EEELQMIQPEKLLLVTVGKTATLHCTVTSLLPVGPVQWFRGVGPGRELIYNQKEGHFPRVTTVSDLTHRNNMDFSIRISSITPADVGTYYCVKFRKGSPEDVEFKSGPGTEMALGAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 483 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 369 - protein, 621 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 566 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 371 - protein, 553 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSMSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDPVKLTCQVEHDGQPAVSKSHDLKVSAHPKEQGSNTAAENTGSNERNIYGACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEHHHHHH

SEQ ID NO: 372 - protein, 455 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEHHHHHH

SEQ ID NO: 373 - protein, 494 aa, synthetic construct
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSHHHHHH

SEQ ID NO: 374 - protein, 505 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSGGGGSGGGGSEEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGSHHHHHH

SEQ ID NO: 375 - protein, 508 aa, synthetic construct
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGSHHHHHH

SEQ ID NO: 376 - protein, 784 aa, synthetic construct
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGSHHHHHH

SEQ ID NO: 377 - protein, 370 aa, synthetic construct
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGSHHHHHH

SEQ ID NO: 378 - protein, 784 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSQVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCGSHHHHHH

SEQ ID NO: 379 - protein, 450 aa, synthetic construct
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 380 - protein, 213 aa, synthetic construct
QIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 381 - protein, 490 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAYSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPS

SEQ ID NO: 382 - protein, 489 aa, synthetic construct
QIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPS

SEQ ID NO: 383 - protein, 370 aa, synthetic construct
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCGGGGSGGGGSGGGGSGGGGSEEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSGSHHHHHH

SEQ ID NO: 384 - protein, 452 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 385 - protein, 590 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 386 - protein, 452 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 387 - protein, 590 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 388 - protein, 590 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 389 - protein, 590 aa, synthetic construct
EEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 390 - protein, 590 aa, synthetic construct
EEELQVIQPDKSVSVAAGESAILHCTVTSLIPVGPIQWFRGAGPARELIYNQKEGHFPRVTTVSESTHRENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 391 - protein, 590 aa, synthetic construct
EEELQVIQPDKSVSVAAGESAILHCTVTSLIPVGPIQWFRGAGPARELIYNQHEGHFPRVTTVSESTKRENMDFSISISAITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 392 - protein, 610 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 393 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 394 - protein, 351 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 395 - protein, 351 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 396 - protein, 351 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 397 - protein, 351 aa, synthetic construct
EEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 398 - protein, 351 aa, synthetic construct
EEELQVIQPDKSVSVAAGESAILHCTVTSLIPVGPIQWFRGAGPARELIYNQKEGHFPRVTTVSESTHRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 399 - protein, 351 aa, synthetic construct
EEELQVIQPDKSVSVAAGESAILHCTVTSLIPVGPIQWFRGAGPARELIYNQHEGHFPRVTTVSESTKRENMDFSISISAITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 400 - protein, 489 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 401 - protein, 489 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 402 - protein, 489 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTHRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 403 - protein, 489 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 404 - protein, 627 aa, synthetic construct
EEELQVIQPDKSVSVAAGESAILHCTVTSLIPVGPIQWFRGAGPARELIYNQHEGHFPRVTTVSESTKRENMDFSISISAITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISAITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISAITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 405 - protein, 451 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 406 - protein, 490 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 407 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 408 - protein, 490 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 409 - protein, 447 aa, synthetic construct
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 410 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 411 - protein, 454 aa, synthetic construct
EVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 412 - protein, 582 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 413 - protein, 582 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 414 - protein, 215 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 415 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 416 - protein, 345 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 417 - protein, 345 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 418 - protein, 256 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPS

SEQ ID NO: 419 - protein, 256 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPS

SEQ ID NO: 420 - protein, 256 aa, synthetic construct
EEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPS

SEQ ID NO: 421 - protein, 256 aa, synthetic construct
EEELQVIQPDKSVSVAAGESAILHCTVTSLIPVGPIQWFRGAGPARELIYNQKEGHFPRVTTVSESTHRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTHRENMDFSISISNITPADAGTYYCVKFRKGSPDTEFKSGAGTELSVRAKPS

SEQ ID NO: 422 - protein, 256 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPS

SEQ ID NO: 423 - protein, 256 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPS

SEQ ID NO: 424 - protein, 256 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPS

SEQ ID NO: 425 - protein, 256 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTHRENMDFSISISNITPADAGTYYCVKFRKGSPDTEFKSGAGTELSVRAKPS

SEQ ID NO: 426 - protein, 256 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPS

SEQ ID NO: 427 - protein, 256 aa, synthetic construct
EEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPS

SEQ ID NO: 428 - protein, 256 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKHSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKHSPDTEFKSGAGTELSVRAKPS

SEQ ID NO: 429 - protein, 256 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 430 - protein, 394 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPS

SEQ ID NO: 431 - protein, 394 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPS

SEQ ID NO: 432 - protein, 394 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPS

SEQ ID NO: 433 - protein, 394 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPS

SEQ ID NO: 434 - protein, 394 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPS

SEQ ID NO: 435 - protein, 394 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPS

SEQ ID NO: 436 - protein, 394 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPS

SEQ ID NO: 437 - protein, 394 aa, synthetic construct
EEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPS

SEQ ID NO: 438 - protein, 394 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKHSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKHSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKHSPDTEFKSGAGTELSVRAKPS

SEQ ID NO: 439 - protein, 394 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 440 - protein, 245 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 441 - protein, 245 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 442 - protein, 245 aa, synthetic construct
EEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 443 - protein, 245 aa, synthetic construct
EEELQVIQPDKSVSVAAGESAILHCTVTSLIPVGPIQWFRGAGPARELIYNQKEGHFPRVTTVSESTHRENMDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 444 - protein, 245 aa, synthetic construct
EEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 445 - protein, 245 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 446 - protein, 245 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 447 - protein, 383 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 448 - protein, 383 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 449 - protein, 383 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 450 - protein, 383 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 451 - protein, 383 aa, synthetic construct
EEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 452 - protein, 383 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 453 - protein, 383 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 454 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 455 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 456 - protein, 467 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 457 - protein, 467 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 458 - protein, 467 aa, synthetic construct
EEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 459 - protein, 467 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 460 - protein, 467 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 461 - protein, 465 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 462 - protein, 351 aa, synthetic construct
EEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 463 - protein, 351 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 464 - protein, 351 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKHSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 465 - protein, 358 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSDIVMTQSPDSLAVSLGERATINCKSSQSVLYSSNNKNYLAWYQQKPGQPPKLLIYWASTRESGVPARFSGSGSGTDFTLTISSLQPEDFATYYCQQYSTVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 466 - protein, 352 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 467 - protein, 352 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 468 - protein, 220 aa, synthetic construct
DIVMTQSPDSLAVSLGERATINCKSSQSVLYSSNNKNYLAWYQQKPGQPPKLLIYWASTRESGVPARFSGSGSGTDFTLTISSLQPEDFATYYCQQYSTVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 469 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 470 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 471 - protein, 448 aa, synthetic construct
EVQLVESGGGLVQAGGSLRLSCAASGFTFSSYAMSWVRQAPGKGLEWVARIYPTNGYTRYADSVKGRFTISRDNSKNTLYLQMNSLRAEDTAVYYCARGYYATSSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 472 - protein, 447 aa, synthetic construct
QVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 473 - protein, 590 aa, synthetic construct
EEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 474 - protein, 590 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 475 - protein, 590 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKHSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 476 - protein, 586 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEVQLVESGGGLVQAGGSLRLSCAASGFTFSSYAMSWVRQAPGKGLEWVARIYPTNGYTRYADSVKGRFTISRDNSKNTLYLQMNSLRAEDTAVYYCARGYYATSSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 477 - protein, 585 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSQVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 478 - protein, 586 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 479 - protein, 489 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 480 - protein, 489 aa, synthetic construct
EEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 481 - protein, 489 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 482 - protein, 489 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 483 - protein, 490 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 484 - protein, 728 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 485 - protein, 728 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 486 - protein, 728 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 487 - protein, 728 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 488 - protein, 728 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 489 - protein, 866 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 490 - protein, 627 aa, synthetic construct
EEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 491 - protein, 627 aa, synthetic construct
EEELQVIQPDKSVSVAAGESAILHCTVTSLIPVGPIQWFRGAGPARELIYNQKEGHFPRVTTVSESTKHENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEEELQVIQPDKSVSVAAGESAILHCTVTSLIPVGPIQWFRGAGPARELIYNQHEGHFPRVTTVSESTKRENMDFSISISNITPADAGTYYCVKFRKGSPDTEFKSGAGTELSVRAKPSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 492 - protein, 345 aa, synthetic construct
EEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 493 - protein, 345 aa, synthetic construct
EEELQVIQPDKSVSVAAGESAILHCTVTSLIPVGPIQWFRGAGPARELIYNQKEGHFPRVTTVSESTHRENMDFSISISNITPADAGTYYCVKFRKGSPDTEFKSGAGTELSVRAKPSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 494 - protein, 345 aa, synthetic construct
EEELQVIQPDKSVSVAAGESAILHCTVTSLIPHGPIQWFRGAGPARELIYNQKEGHFPRVTTVSESTKRENMDFSISISNITPADAGTYYCVKFRKGSPDTEFKSGAGTELSVRAKPSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 495 - protein, 345 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 496 - protein, 345 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 497 - protein, 345 aa, synthetic construct
EEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 498 - protein, 452 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 499 - protein, 447 aa, synthetic construct
QVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 500 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 501 - protein, 591 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 502 - protein, 591 aa, synthetic construct
EEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 503 - protein, 591 aa, synthetic construct
EEELQVIQPDKSVSVAAGESAILHCTVTSLIPVGPIQWFRGAGPARELIYNQKEGHFPRVTTVSESTHRENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 504 - protein, 591 aa, synthetic construct
EEELQVIQPDKSVSVAAGESAILHCTVTSLIPVGPIQWFRGAGPARELIYNHKEGHFPRVTTVSESTHRENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 505 - protein, 591 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 506 - protein, 586 aa, synthetic construct
EEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSGGGGSGGGGSGGGGSGGGGSQVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 507 - protein, 587 aa, synthetic construct
EEELQVIQPDKSVSVAAGESAILHCTVTSLIPVGPIQWFRGAGPARELIYNHKEGHFPRVTTVSESTKRENMDFSISISNITPADAGTYYCVKFRKGSPDTEFKSGAGTELSVRAKPSGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 508 - protein, 590 aa, synthetic construct
EEELQVIQPDKSVSVAAGESAILHCTVTSLIPVGPIQWFRGAGPARELIYNQKEGHFPRVTTVSESTHHENMDFSISISNITPADAGTYYCVKFRKGSPDTEFKSGAGTELSVRAKPSGGGGSGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 509 - protein, 582 aa, synthetic construct
EEELQVIQPDKSVSVAAGESAILHCTVTSLIPVGPIQWFRGAGPARELIYNQKEGHFPRVTTVSESTKRENHDFSISISNITPADAGTYYCVKFRKGSPDTEFKSGAGTELSVRAKPSGGGGSGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 510 - protein, 582 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGGGGSGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 511 - protein, 607 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 512 - protein, 610 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 513 - protein, 345 aa, synthetic construct
EEELQVIQPDKSVSVAAGESAILHCTVTSLDPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 514 - protein, 345 aa, synthetic construct
EEELQVIQPDKSVSVAAGESAILHCTVTSLEPVGPIQWFRGAGPARELIYNQHEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 515 - protein, 345 aa, synthetic construct
EEELQVIQPDKSVSVAAGESAILHCTVTSLEPVGPIQWFRGAGPARELIYNQKEGHFPRVTTVSESTKHENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 516 - protein, 345 aa, synthetic construct
EEELQVIQPDKSVSVAAGESAILHCTVTSLWPVGPIQWFRGAGPARELIYNQKEGHFPHVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 517 - protein, 345 aa, synthetic construct
EEELQVIQPDKSVSVAAGESAILHCTVTSLEPVGPIQWFRGAGPARELIYNHKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 518 - protein, 345 aa, synthetic construct
EEELQVIQPDKSVSVAAGESAILHCTVTSLEPVGPIQWFRGAGPARELIYNQKEGHFPRVTTVSESTHRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0274921 A1
(43) Pub. Date: Sep. 17, 2026

(54) SIRP VARIANTS AND USES THEREOF

(71) Applicant: ADAPTOCUE, LLC, Boston (MA)

(72) Inventor: Wangzhi LI, Boston, MA (US)

(73) Assignee: ADAPTOCUE, LLC, Boston (MA)

(21) Appl. No.: 19/167,489

(22) PCT Filed: Mar. 21, 2024
(86) PCT No.: PCT/CN2024/082942

§ 371 Date: Sep. 22, 2025

(30) Foreign Application Priority Data
Mar. 22, 2023 (WO) PCT/CN2023/083165

(51) Int. Cl. C07K 14/705; A61K 45/06

(52) U.S. Cl. CPC C07K 14/70596; A61K 45/06; C07K 2319/30

(57) ABSTRACT

Provided herein are variants of CD47-binding SIRP IgV, comprising a substitution, wherein the variants exhibit higher binding affinity to CD47 at an acidic pH than at physiological pH, and the uses thereof.

### CROSS REFERENCE TO RELATED APPLICATIONS

This application claims the benefit of, and prior ity to, PCT Application No. PCT/CN2023/083165, filed on Mar. 22, 2023. The content of these applications is incorporated herein by reference in their entirety for all purposes.

### TECHNICAL FIELD

The present disclosure generally relates to variants of CD47-binding SIRP IgV, comprising a substitution, and the uses thereof.

### BACKGROUND

Phagocytosis is a major mechanism to clear pathogens, disease cells, dying cells, and cell debris by professional phagocytes such as macrophages, monocytes, dendritic cells, and granulocytes. The trigger of phagocytosis of a target cell or agent by a phagocyte is driven by an integration and imbalance of pro-phagocytic signal and anti-phagocytic signals. The protein CD47 (cluster of differentiation 47), also known as IAP (integrin-associated protein), functions as a major anti-phagocytic signal that inhibits phagocytosis of the cells expressing CD47 through interacting with the Signal Regulatory Protein (SIRP) receptor, most notably SIRPα, on phagocytes such as macrophages and triggering an “anti-phagocytic” signal. CD47 is ubiquitously expressed in normal tissues and cells, and plays an important role in sparing the normal cells from phagocytosis. Disease cells such as cancer cells, often with upregulation of CD47 expression, however hijack this mechanism to escape normal immune control and clearance by phagocytes. CD47 has been shown to be highly expressed and associated with adverse prognosis in a wide variety of cancers (Zhang et al., Front Immunol, 2020). Blocking the CD47 “anti-phagocytic” signal by CD47 binders, together with co-presence of sufficient “pro-phagocytic” signal, is shown to promote phagocytosis of a variety of CD47-expressing cancer cells, and presents an attractive therapeutic strategy to treat a broad range of cancers. Additionally, upregulated CD47 is also found in atherosclerotic plaques to prevent effective removal of the diseased tissue, and administration of CD47-blocking antibodies can normalize this defective phagocytic clearance and ameliorates atherosclerosis (Kojima et al., Nature, 2016). Blockade of CD47 is also shown to reduce lung fibrosis in vivo (Wernig et al., Proc Natl Acad Sci USA, 2017), promote necroptotic hepatocyte clearance by liver macrophages and decreases hepatic fibrosis and attenuate liver fibrosis in experimental non-alcoholic steatohepatitis (NASH) models {Shi, 2022 #838} {Gwag, 2022 #839}. Similarly, CD47 is reported to prevent elimination of diseased fibroblasts in fibrotic scleroderma and blocking CD47 reversed skin fibrosis in combination with IL-6 blockade (Lerbs et al., JCI Insight, 2020). Furthermore, increased expression of CD47 is reported in other diseases such as Gaucher disease, multiple sclerosis and stroke, among others (Gheibihayat et al., Molecules, 2021). Also, up-regulation of CD47 expression is found during infection and blockade of CD47 enhances innate and adaptive immune response to infection (Zahavi et al., Antibodies (Basel), 2020). Thus, anti-CD47 therapeutics also holds promise for treating non-cancer diseases such as atherosclerosis, fibrosis and infection.

However, development of anti-CD47 therapeutics is hampered by the ubiquitous expression of CD47 across normal tissues and cells, which present safety risk to CD47-expressing normal tissues and also a huge antigen sink sequestering the anti-CD47 therapeutics from diseased tissue. Thus, it's desirable to develop novel CD47 binders and CD47-targeting therapeutics that can address these issues. Described herein are compositions of CD47-binding protein domains as well as proteins, proteins conjugates and synthetic receptor constructs comprising said CD47-binding SIRP IgV domains for differentially targeting CD47-expressing disease tissues/cells, and methods of use and production related thereto.

### SUMMARY

In one aspect, the present disclosure provides a variant of CD47-binding IgV extracellular domain of the Signal-regulatory protein (SIRP), wherein the variant shows higher binding affinity to CD47 at an acidic pH than at physiological pH, wherein an acidic pH is less than pH 7.0, e.g. pH 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, and physiological pH is pH 7.2 to 7.5.

### SIRP IgV Monomer

In some embodiments, a IgV extracellular domain of the Signal-regulatory protein (SIRP), hereafter referred to as SIRP IgV, comprises a IgV extracellular domain derived from a Signal-regulatory protein (SIRP) family protein selected from the group consisting of SIRPα, SIRPβ and SIRPγ.

In some embodiments, the SIRP IgV comprises an amino acid sequence selected from the group consisting of SEQ ID NO: 3-21. In some embodiments, the SIRP IgV is selected from the group consisting of SIRPαV1 (SEQ ID NO: 3), SIRPαV2 (SEQ ID NO: 4), SIRPαV3 (SEQ ID NO: 5), SIRPαV4 (SEQ ID NO: 6), SIRPαV5 (SEQ ID NO: 7), SIRPαV6 (SEQ ID NO: 8), SIRPαV7 (SEQ ID NO: 9), SIRPαV8 (SEQ ID NO: 10), SIRPαV9 (SEQ ID NO: 11), SIRPαV10 (SEQ ID NO: 11), SIRPβ1 (SEQ ID NO: 1), SIRPβ1-VQ (SEQ ID NO: 12), SIRPβ1-VQP (SEQ ID NO: 13), SIRPβ1-VQM (SEQ ID NO: 14), SIRPβ1-VQPM (SEQ ID NO: 15), SIRPβ1-TVQS (SEQ ID NO: 16), SIRPβ2 (SEQ ID NO: 2), SIRPβ2-D (SEQ ID NO: 17), SIRPγ (SEQ ID NO: 18), SIRPγ-Q (SEQ ID NO: 19), SIRPγ-D (SEQ ID NO: 20) and SIRPγ-QD (SEQ ID NO: 21).

In some embodiments, the present disclosure provides a variant of CD47-binding IgV extracellular domain of SIRPα or SIRPβ, wherein the variant comprises mutation at one or more amino acid residues selected from the group consisting of I31, Q52, K53, K68 and R69 (residue position numbering based on the residue position in SEQ ID NO: 1 to 17), and wherein the variant shows higher binding affinity to CD47 at an acidic pH than at physiological pH.

In some embodiments, the present disclosure provides a variant of CD47-binding IgV extracellular domain of SIRPγ, wherein the variant comprises mutation at one or more amino acid residues selected from the group consisting of L31, Q52, K53, K68 and R69 (residue position numbering based on the residue position in SEQ ID NO: 18 to 21), and wherein the variant shows higher binding affinity to CD47 at an acidic pH than at physiological pH.

In some embodiments, the present disclosure provides a variant of CD47-binding SIRP IgV, wherein the variant comprises one or more substitutions selected from the group consisting of K53H, R69H, K68H, Q52H, and I31E/L31E or I31D/L31D wherein the “/” indicates different alternative mutation change at the corresponding amino acid residue position across SIRPα, SIRPβ and SIRPγ IgV domains, due to different parent amino acid residues and/or position numbering for the mutation at the corresponding position according to the SIRPα, SIRPβ and SIRPγ IgV amino acid sequences provided in SEQ ID NO: 1 to 21, and wherein the variant shows higher binding affinity to CD47 at an acidic pH than at physiological pH. In some embodiments, a variant of CD47-binding SIRP IgV comprises a substitution of Q52H, K53H, K68H, R69H, I31E/L31E or I31D/L31D. In certain embodiments, a variant of CD47-binding SIRP IgV comprises a substitution selected from the group consisting of Q52H+K68H, K53H+K68H, R69H+K68H, K53H+Q52H, R69H+Q52H, K53H+I31E/L31E, R69H+I31E/L31E, K68H+I31E/L31E, Q52H+I31E/L31E, Q52H+K68H+I31E/L31E, K53H+I31D/L31D, R69H+I31D/L31D, K68H+I31D/L31D, Q52H+I31D/L31D, and Q52H+K68H+I31D/L31D, wherein the “+” indicates concurrent combination mutations in a single SIRP IgV monomer domain. In certain embodiments, a variant of CD47-binding SIRP IgV comprises a substitution selected from the group consisting of K53H+K68H+Q52H, and R69H+K68H+Q52H.

In some embodiments, the present disclosure provides a variant of CD47-binding SIRP IgV, wherein the variant comprises a substitution of I31E, I31D, L31E or L31D, and wherein the variant shows higher binding affinity to CD47 at an acidic pH than at physiological pH.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, E54D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79B/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D, wherein the “/” indicates different alternative mutation change at the corresponding amino acid residue position across SIRPα, SIRPβ and SIRPγ IgV domains, due to different parent amino acid residues and/or position numbering for the mutation at the corresponding position, according to the SIRPα, SIRPβ and SIRPγ IgV amino acid sequences provided in SEQ ID NO: 1 to 21.

In some embodiments, a variant of CD47-binding SIRP IgV comprises a substitution of I31E/L31E, wherein the variant further comprises one or more substitutions selected from the group consisting of E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52H, Q52E, Q52D, Q52G, K53H, K53R, K53E, K53D, E54H, E54D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68H, K68R, K68A, K68E, K68D, K68I, K68T, R69H, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D, wherein the “/” indicates different alternative mutation change at the corresponding amino acid residue position across SIRPα, SIRPβ and SIRPγ IgV domains, due to different parent amino acid residues and/or position numbering for the mutation at the corresponding position, according to the SIRPα, SIRPβ and SIRPγ IgV amino acid sequences provided in SEQ ID NO: 1 to 21.

In some embodiments, a variant of CD47-binding SIRP IgV comprises a substitution of I31D/L31D, wherein the variant further comprises one or more substitutions selected from the group consisting of E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52H, Q52E, Q52D, Q52G, K53H, K53R, K53E, K53D, E54H, E54D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68H, K68R, K68A, K68E, K68D, K68I, K68T, R69H, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/0115H, R114E/R115E/G115E, R114D/R115D/G115D, K116H/K117H, K116E/K117E, and K116D/K117D

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, I31H/L31H, I31Y/L31Y, I31W/L31W, V33H, P35H, Q37H/L37H, R40H, R46H, N51H, R59H, S66H/L66H, E70H/N70H, M72H/L72H, K96H, K96R, G97H, S98H, P99H, and K104H/K105H.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises an additional mutation of I31E or L31E, wherein the additional mutation of I31E/L31E further lowes the binding of the SIRP IgV variant to CD47 at physiological pH. In some embodiments, a variant of CD47-binding SIRP IgV comprises a mutation of K53H+I31E/L31E. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises an additional mutation of I31D or L31D, wherein the additional mutation of I31D/L31D further lowes the binding of the SIRP IgV variant to CD47 at physiological pH.

In some embodiments, a variant of CD47-binding SIRP IgV comprises a mutation of I31E+Q37H or L31E+L37H, wherein the variant exhibits further lowered binding to CD47 at physiological pH than a counterpart variant of CD47-binding SIRP IgV comprises I31E or L31E mutation only. In some embodiments, a variant of CD47-binding SIRP IgV comprises a mutation of I31D+Q37H or L31D+L37H, wherein the variant exhibits further lowered binding to CD47 at physiological pH than a counterpart variant of CD47-binding SIRP IgV comprises I31D or L31D mutation only.

In some embodiments, a variant of CD47-binding IgV extracellular domain of SIRPγ (SIRPγ IgV) comprises one or more mutation selected from the group consisting of K53H, R69H, Q52H, K68H, L31E and L31D. In further embodiment, the foregoing variant SIRPγ IgV comprises an additional mutation of N101D, L37Q, N101D+L37Q, L37H or N101D+L37H. In some embodiments, a variant SIRPγ IgV comprises a mutation of K53H, R69H, Q52H, K68H, Q52H+K68H, L31E, L31D, K53H+L31E, or K53H+L31D. In some embodiments, a variant SIRPγ IgV comprises a mutation of K53H+N101D, K53H+N101D+L37Q, K53H+N101D+L31E, K53H+N101D+L37Q+L31E, K53H+N101D+L31D, or K53H+N101D+L37Q+L31D. In some embodiments, a variant SIRPγ IgV comprises a mutation of R69H+N101D, Q52H+N101D, K68H+N101D, R69H+N101D+L37Q, Q52H+N101D+L37Q, or K68H+N101D+L37Q. In some embodiments, a variant SIRPγ IgV comprises a mutation of K53H+L37Q, R69H+L37Q, Q52H+L37Q, or K68H+L37Q.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of L4V, L4I, V6I/M6I, V6L/M6L, A21V, V27I/A27I, V27L/A27L, V27Q/A27Q, I31E/L31E, I31D/L31D, I31R/L31R, I31K/L31K, I31F/L31F, I31T/L31T, I31S/L31S, I31L, V33I, P35G, P35N, Q37A/L37A, Q37V/L37V, Q37W/L37W, E47V, E47L, E47Q, E47Y, Q52G, Q52E, K53R, E54Q, E54S, E54D, E54N, E54G, E54P, H56P, H56R, H56Y, V63I, V63A, E65H/D65H, E65R/D65R, S66T/L66T, S66G/L660, S66Q/L66Q, S66A/L66A, S66E/L66E, S66W/L66W, T67E, T67W, K68R, K68A, K68E, K68I, K68T, E70D/N70D, M72I/L72I, M72N/L72N, M72W/L72W, M72R/L72R, S77N/R77N, S77K/R77K, S79H/G79H, N80A, N80S, N80Q, V92I, V92S, V92N, F94L, F94V, F103V/F104V, and F103I/F104I.

In some embodiments, a variant of CD47-binding SIRP IgV comprises additional mutation, wherein the additional mutation comprises one or more substitution selected from the group consisting of V6I/M6I, V27I/A27I, V27Q/A27Q, I31R/L31R, I31T/L31T, P35G, P35N, Q37A/L37A, Q37V/L37V, Q37W/L37W. E47Y, Q52E, E54P, H56Y, H56P, S66E/L66E, S66W/L66W, S66Q/L66Q, T67E, T67W, K68A, K68E, K68I, K68T, M72I/L72I, M72N/L72N, M72W/L72W, M72R/L72R, N80A and V92N.

In some embodiments, a variant of CD47-binding SIRP IgV comprises additional mutation, wherein the additional mutation comprises Q37H/L37H. In some embodiments, a variant of CD47-binding SIRP IgV comprises additional mutation, wherein the additional mutation comprises N80A, N80G, N80S, or N80Q. In some embodiments, a variant of CD47-binding SIRP IgV comprises additional mutation, wherein the additional mutation comprises R59H, I31H/L31H, I31Y/L31Y or I31W/L31W.

In some embodiments, a variant of a CD47-binding SIRP IgV comprises one or more substitutions selected from the group consisting of I31E/L31E, I31D/L31D, V33H, P35H, Q37H/L37H, R40H, R46H, N51H, S66H/L66H, E70H/N70H, M72H/L72H, K96R, K96H, G97H, S98H, P99H, and K104H/K105H.

In some embodiments, a variant of a CD47-binding SIRP IgV comprises one or more substitutions selected from the group consisting of I31H/L31H, I31Y/L31Y, I31W/L31W, R59H, N80A, N80G, N80S, and N80Q. In some embodiments, a variant of a CD47-binding SIRP IgV comprises a mutation of I31W/L31W. In some embodiments, a variant of a CD47-binding SIRP IgV comprises a mutation of I31Y/L31Y. In some embodiments, a variant of a CD47-binding SIRP IgV comprises a mutation of I31W/L31W+R59H. In some embodiments, a variant of a CD47-binding SIRP IgV comprises a mutation of I31Y/L31Y+R59H. In some embodiments, a variant of a CD47-binding SIRP IgV comprises a mutation of I31W+N80A/G/S/Q. In some embodiments, a variant of a CD47-binding SIRP IgV comprises a mutation of I31Y+N80A/G/S/Q.

In some embodiments, a variant of CD47-binding SIRP IgV comprising an amino acid sequence at least 80%, 85%, 90%, 95%, 96%, 97%, 98%, 99% identical to SEQ ID NO: 3 to 81, preferably SEQ ID NO: 22-81, wherein the variant shows higher binding affinity to CD47 at an acidic pH than at physiological pH.

### SIRP IgV Multimer Polypeptide

In one aspect, the present disclosure provides a SIRP IgV multimer polypeptide comprising two, three, four or more CD47-binding SIRP IgV monomers that are linked together serially from the N-terminal to the C-terminal of the polypeptide. In some embodiments, the CD47-binding SIRP IgV monomer comprises an amino acid sequence selected from the group consisting of SEQ ID NO: 3 to 81. In some embodiments, the two or more CD47-binding SIRP IgV monomers are serially linked through a linker. In certain embodiments, the linker comprises (GGGGS)n, wherein n=1, 2, 3, 4, 5, or 6. In certain embodiments, the linker is GGGGSGGGGSGGGGSGGGGS (SEQ ID NO: 121).

In some embodiments, the SIRP IgV multimer polypeptide comprises two CD47-binding SIRP IgV variant monomers comprising the same mutation selected from the group consisting of K53H, R69H, Q52H, K68H, I31D/L31D, Q52H+K68H, K53H+K68H, R69H+K68H, K53H+Q52H, R69H+Q52H, K53H+I31E/L31E, R69H+I31E/L31E, K68H+I31E/L31E, Q52H+I31E/L31E, Q52H+K68H+I31E/L31E, I31E/L31E, K53H+I31D/L31D, R69H+I31D/L31D, K68H+I31D/L31D, Q52H+I31D/L31D, and Q52H+K68H+I31D/L31D In certain embodiments, the SIRP IgV multimer polypeptide comprises two CD47-binding SIRP IgV variant monomers comprising different mutation, wherein the mutation of the N-terminal monomer and the C-terminal monomer of the SIRP IgV multimer polypeptide comprises a substitution of K53H-R69H (i.e. the N-terminal monomer comprising K53H mutation and the C-terminal monomer comprising R69H mutation), R69H-K53H, Q52H-R69H, R69H-Q52H, K68H-R69H, R69H-K68H, Q52H-K53H, K53H-Q52H, K68H-K53H, K53H-K68H, Q52H-K68H, K68H-Q52H, Q52H+K68H-R69H, R69H-Q52H+K68H, Q52H+K68H-K53H, or K53H-Q52H+K68H, wherein the “+” indicates concurrent mutation in a single SIRP IgV monomer, while the “-” indicates separate mutation of two neighboring SIRP IgV monomers serially linked from the N-terminal to the C-terminal, wherein the mutation from the left to the right of the “−” indicates the mutation from the N-terminal monomer to the C-terminal monomer. In further embodiment, the foregoing N-terminal monomer and/or C-terminal monomer comprise an additional mutation of I31E/L31E. In further embodiment, the foregoing N-terminal monomer and/or C-terminal monomer comprise an additional mutation of I31D/L31D. In certain embodiment, the N-terminal monomer comprises a substitution of I31E/L31E and the C-terminal monomer comprises a substitution selected from the group consisting of K53H, R69H, Q52H, K68H and Q52H+K68H, or vice versa. In certain embodiment, the N-terminal monomer comprises a substitution of I31D/L31D and the C-terminal monomer comprises a substitution selected from the group consisting of K53H, R69H, Q52H, K68H and Q52H+K68H, or vice versa.

In some embodiments, the SIRP IgV multimer polypeptide comprises three CD47-binding SIRP IgV variant monomers, wherein the three monomers comprise the same mutation. In certain embodiments, the SIRP IgV multimer polypeptide comprises three CD47-binding SIRP IgV variant monomers comprising the same mutation selected from the group consisting of K53H, R69H, Q52H, K68H, I31E/L31E, I31D/L31D, Q52H+K68H, K53H+K68H, R69H+K68H, K53H+Q52H, R69H+Q52H, K53H+I31E/L31E, R69H+I31E/L31E, K68H+31E/L31E, Q52H+I31E/L31E, Q52H+K68H+I31E/L31E, K53H+I31D/L31D, R69H+I31D/L31D, K68H+31D/L31D, Q52H+I31D/L31D, and Q52H+K68H+I31D/L31D. In certain embodiments, the SIRP IgV multimer polypeptide comprises three CD47-binding SIRP IgV variant monomers comprising different mutation, wherein the mutation from the N-terminal monomer to the middle monomer to the C-terminal monomer of the SIRP IgV multimer polypeptide comprises a substitution of K53H-K53H-R69H (i.e. the N-terminal monomer comprising K53H, the middle monomer comprising K53H, and the C-terminal monomer comprising R69H), R69H-K53H-K53H, K53H-R69H-K53H, K53H-R69H-R69H, R69H-R69H-K53H, R69H-K53H-R69H, K53H-K53H-Q52H, Q52H-K53H-K53H, K53H-Q52H-K53H, K53H-K53H-K68H, K68H-Q53H-K53H, K53H-K68H-K53H, Q52H-R69H-R69H, R69H-R69H-Q52H, R69H-Q52H-R69H, K68H-R69H-R69H, R69H-R69H-K68H, or R69H-K68H-R69H. In further embodiment, the foregoing N-terminal monomer, middle monomer and/or C-terminal monomer comprise an additional mutation of I31E/L31E. In further embodiment, the foregoing N-terminal monomer, middle monomer and/or C-terminal monomer comprise an additional mutation of I31D/L31D.

In some embodiments, the SIRP IgV multimer polypeptide comprises four CD47-binding SIRP IgV variant monomers, wherein the four monomers comprise the same mutation. In certain embodiments, the SIRP IgV multimer polypeptide comprises four CD47-binding SIRP IgV variant monomers comprising the same mutation selected from the group consisting of I31E/L31E, I31D/L31D, Q52H+K68H, K53H+K68H, R69H+K68H, K53H+Q52H, R69H+Q52H, K53H+I31E/L31E, R69H+I31E/L31E, K68H+I31E/L31E, Q52H+I31E/L31E, Q52H+K68H+I31E/L31E, K53H+I31D/L31D, R69H+I31D/L31D, K68H+I31D/L31D, Q52H+I31D/L31D, and Q52H+K68H+I31D/L31D. In certain embodiments, the SIRP IgV multimer polypeptide comprises four CD47-binding SIRP IgV variant monomers comprising different mutation, wherein the mutation from the N-terminal monomer to the C-terminal monomer serially comprises a substitution of K53H-K53H-R69H-R69H, R69H-R69H-K53H-K53H, R69H-R69H-Q52H-Q52H, Q52H-Q52H-R69H-R69H, R69H-R69H-K68H-K68H, K68H-K68H-R69H-R69H, K53H-K53H-Q52H-Q52H, Q52H-Q52H-K53H-K53H, R69H, K53H-K53H-K68H-K68H, K68H-K68H-K53H-K53H, K53H-R69H-R69H-R69H, R69H-R69H-R69H-K53H, Q52H-R69H-R69H-R69H, R69H-R69H-R69H-Q52H, K68H-R69H-R69H-R69H, R69H-R69H-R69H-K68H, R69H-K53H-K53H-K53H, K53H-K53H-K53H-R69H, Q52H-K53H-K53H-K53H, K53H-K53H-K53H-Q52H, K68H-K53H-K53H-K53H, or K53H-K53H-K53H-K68H. In further embodiment, one, two, three or four of the foregoing monomers comprise an additional mutation of I31E/L31E. In further embodiment, the one, two, three or four of the foregoing monomers comprise an additional mutation of I31D/L31D.

### Fusion Polypeptide

In one aspect, the present disclosure provides a fusion polypeptide comprising a CD47-binding SIRP IgV domain comprising one or more CD47-binding SIRP IgV monomers of present disclosure, and a non-CD47 binding domain. In some embodiments, the fusion polypeptide comprises a CD47-binding SIRP IgV domain comprising one, two, three, four, five, or six CD47-binding SIRP IgV monomers. In some embodiments, the fusion polypeptide comprises two, three, four or more CD47-binding SIRP IgV monomers, wherein the monomers comprise the same or different amino acid sequence and/or mutation. In some embodiments, the CD47-binding SIRP IgV domain of the fusion polypeptide comprises a SIRP IgV multimer polypeptide of present disclosure, wherein the SIRP IgV multimer polypeptide comprises two, three, four or more serially linked CD47-binding SIRP IgV monomers comprising the same or different amino acid sequence and/or mutation. In some embodiments, the fusion polypeptide comprises at least two CD47-binding SIRP IgV monomers, wherein the two SIRP IgV monomers are separated by a non-SIRP IgV domain, wherein one SIRP IgV monomer is linked to the N-terminal of the non-SIRP IgV domain and the other SIRP IgV monomer is linked to the C-terminal of the non-SIRP IgV domain. In some embodiments, the fusion polypeptide comprises at least two CD47-binding SIRP IgV monomers, wherein the two SIRP IgV monomers are separated by a non-SIRP IgV domain, wherein the non-SIRP IgV domain is a Fc region or CH2 region or CH3 region or fragment thereof, an antibody light chain or VL or CL or fragment thereof, or an antibody heavy chain or VH or CH1 or fragment thereof. In some embodiments, the fusion polypeptide comprises at least two CD47-binding SIRP IgV monomers, wherein the two SIRP IgV monomers are separated by a non-SIRP IgV domain, wherein the non-SIRP IgV domain is a VHH antibody or nanobody, an immunoglobulin new antigen receptor (IgNAR), or other single-chain domain antibody, D-Domain, nanofitin, adnectin, knottin, kunitz, avimer, affilin, ADAPT or an anticalin, DARPIN, affibody, affimer, centryin domain, fibronectin domain, D-Domain, nanofitin, adnectin, knottin, kunitz, avimer, affilin, ADAPT or other alternative scaffold known in the arts, for example as described in {Luo, 2022 #919}, to function as antigen binding domain that binds to an antigen. In some embodiments, the fusion polypeptide comprises at least two CD47-binding SIRP IgV monomers, wherein the two SIRP IgV monomers are separated by a non-SIRP IgV domain, wherein the non-SIRP IgV domain is a peptide, a receptor or fragment thereof, a ligand or fragment thereof, a cytokine or fragmente thereof, a chemokine or fragment thereof, or a growth factor or fragmente thereof. In certain embodiments, the CD47-binding SIRP IgV monomer comprises an amino acid sequence selected from the group consisting of SEQ ID NO: 3 to 81. In some embodiments, the CD47-binding SIRP IgV domain of the fusion polypeptide comprises a SIRP IgV multimer polypeptide of present disclosure comprising two CD47-binding SIRP IgV monomers. In certain embodiments, the SIRP IgV multimer polypeptide comprising two CD47-binding SIRP IgV monomers comprises an amino acid sequence selected from the group consisting of SEQ ID NO: 82 to 96 and SEQ ID NO: 418 to 425. In some embodiments, the CD47-binding SIRP IgV domain of the fusion polypeptide comprises three CD47-binding SIRP IgV monomers. In certain embodiments, the SIRP IgV multimer polypeptide comprising three CD47-binding SIRP IgV monomers comprises an amino acid sequence selected from the group consisting of SEQ ID NO: 97 to 109 and SEQ ID NO: 430 to 435. In some embodiments, the CD47-binding SIRP IgV domain of the fusion polypeptide comprises four CD47-binding SIRP IgV monomers. In certain embodiments, the SIRP IgV multimer polypeptide comprising four CD47-binding SIRP IgV monomers comprises an amino acid sequence selected from the group consisting of SEQ ID NO: 110 to 113. In some embodiments, the SIRP IgV monomers of the fusion polypeptide comprise the same amino acid sequence and/or mutation. In some embodiments, the SIRP IgV monomers of the fusion polypeptide comprise different amino acid sequence and/or mutation.

In some embodiments, the fusion polypeptide comprises one or more additional domains that bind to a non-CD47 antigen. In some embodiments, the non-CD47 binding domain of the fusion polypeptide binds to a soluble antigen, an aggregate of antigen, a surface antigen on a cell, a surface antigen on a microbe, or a hapten. In some embodiments, the non-CD47 binding domain of the fusion polypeptide binds to an aggregate of antigen, wherein the aggregate of antigen comprises aggregate of proteins and/or lipids. In some embodiments, the non-CD47 binding domain of the fusion polypeptide binds to a surface antigen on a cell, wherein the cell comprises a diseased cell, an infected cell or an effector cell. In certain embodiments, the diseased cell is a cancer cell, a fibrotic cell, an atherosclerotic cell, an inflammatory cell or a senescent cell. In certain embodiments, the infected cell is a cell infected by bacterium, fungus, virus and/or parasite. In certain embodiments, the effector cell is a myeloid cell, a lymphocyte or a granulocyte. In certain embodiments, the effector cell is a macrophage, a monocyte, a dendritic cell, a B cell, a T cell, a NK cell, a NKT cell, an innate lymphoid cell (ILC), a neutrophil, and/or a mast cell. In certain embodiments, the non-CD47 binding domain of the fusion polypeptide binds to a surface antigen on a microbe, wherein the microbe is a bacterium, a fungus, a protozoa, or a virus. In certain embodiments, the non-CD47 binding domain of the fusion polypeptide binds to a hapten, wherein the hapten comprises a chelator that binds to a radionuclide or radiometal. In certain embodiments, the chelator is a macrocyclic chelator, an acyclic chelator or a hybrid chelator. In certain embodiments, the hapten comprises a hapten peptide histamine-succinyl-glycine (HSG). In some embodiments, the non-CD47 binding domain of the fusion polypeptide comprises a peptide or polypeptide with antigen-binding, a ligand or fragment thereof with receptor binding, a receptor or fragment thereof with ligand binding, a heavy chain variable domain (VH), a light chain variable domain (VL), a single chain fragment variable (scFv) comprising a VH and a VL, a single chain Fab domain (scFab), a VHH antibody or nanobody, a VH single chain antibody (VH dAb) or a VL single chain antibody (VL dAb), an immunoglobulin new antigen receptor (IgNAR), or other single-chain domain antibody, D-Domain, nanofitin, adnectin, knottin, kunitz, avimer, affilin, ADAPT or an anticalin, DARPIN, affibody, affimer, centryin domain, fibronectin domain, D-Domain, nanofitin, adnectin, knottin, kunitz, avimer, affilin, ADAPT or other alternative scaffold known in the arts, for example as described in {Luo, 2022 #919}, to function as antigen binding domain that binds to the non-CD47 antigen.

In some embodiments, the fusion polypeptide comprises a Fe region or its functional fragment thereof. The functional fragment comprises CH2 and/or CH3. The Fc region is selected from the group consisting of human IgG1, IgG2, IgG3, IgG4, IgA, IgM, IgE, and its variants. In some embodiments, the Fc region is a human IgG1 with wild-type effector function. In some embodiments, the Fc region is a human IgG1 with enhanced effector function. In some embodiments, the Fe region is a human IgG4 with a mutation of S228P, according to the EU numbering scheme. In some embodiments, the Fc region is a human IgG2.

In some embodiments, the fusion polypeptide comprises a CD47-binding SIRP IgV domain and a non-CD47 binding domain, wherein the CD47-binding SIRP IgV domain is linked to the N-terminal and/or the C-terminal of the non-CD47 binding domain of the fusion polypeptide. In some embodiments, the fusion polypeptide comprises a CD47-binding SIRP IgV domain and a Fc region, wherein the CD47-binding SIRP IgV domain is linked to the N-terminal and/or the C-terminal of the Fc region of the fusion polypeptide.

### Fusion Protein

In one aspect, the present disclosure provides a fusion protein comprising one said fusion polypeptide of present disclosure. In one aspect, the present disclosure provides a fusion protein comprising two or more said fusion polypeptides of present disclosure. In some embodiments, a fusion protein comprises two or more said fusion polypeptides, wherein the two or more said fusion polypeptides comprise the same number of CD47-binding SIRP IgV monomers. In certain embodiments, a protein comprises two or more said fusion polypeptides, wherein the two or more fusion polypeptides comprise different number of CD47-binding SIRP IgV monomers. In certain embodiments, a protein comprises two or more said fusion polypeptides, wherein the composition of mutation of the CD47-binding SIRP IgV monomers of the two or more fusion polypeptides is the same. In certain embodiments, a protein comprises two or more said fusion polypeptides, wherein the composition of mutation of the CD47-binding SIRP IgV variant monomers of the two or more fusion polypeptides is different.

In some embodiments, a fusion protein comprising one or more said fusion polypeptides of present disclosure comprises in total one, two, three or four CD47-binding SIRP IgV monomers of present disclosure.

In some embodiments, a fusion protein comprising one or more said fusion polypeptides comprises three, four or more CD47-binding SIRP IgV monomers in total and a human IgG1 Fe with wild type or enhanced effector function, wherein the CD47-binding SIRP IgV monomers preferably comprise a substitution of K53H, R69H, K53H+I31E, or R69H+I31E.

In some embodiments, a fusion protein comprising one or more said fusion polypeptides comprises a CD47-binding SIRP IgV domain, wherein the CD47-binding SIRP IgV domain comprises one SIRP IgV monomer. In certain embodiments, the CD47-binding SIRP IgV domain comprises one SIRP IgV monomer comprising an amino acid sequence selected from the group consisting of SEQ ID NO: 3 to 81. In some embodiments, a fusion protein comprising one or more said fusion polypeptides comprises a CD47-binding SIRP IgV domain, wherein the CD47-binding SIRP IgV domain comprises two, three, four or more SIRP IgV monomers. In certain embodiments, a fusion protein comprising one or more said fusion polypeptides comprises a SIRP IgV multimer polypeptide of present disclosure comprising two, three or four SIRP IgV monomers. In certain embodiments, the SIRP IgV multimer polypeptide of the fusion protein comprises an amino acid sequence selected from the group consisting of SEQ ID NO: 82 to 113, SEQ ID NO: 418 to 425 and SEQ ID NO: 430 to 435.

In some embodiments, a fusion protein comprises two said fusion polypeptides comprising a Fc region, wherein the Fc region of the two fusion polypeptides form a homodimeric Fc. In certain embodiments, a fusion protein comprises two said fusion polypeptides comprising a Fc region, wherein the Fe region of the two fusion polypeptides comprise mutation that promote formation of heterodimeric Fc. In some embodiments, a fusion protein comprises one or more said fusion polypeptides is configured in one of the exemplary formats of FV-1 to 195 as set forth in FIGS. 2 to 12.

In some embodiments, the Fc region is selected from the group consisting of human IgG1, IgG2, IgG3, IgG4, IgA, IgM, IgE, and its modification.

In some embodiments, the fusion protein further comprises at least one additional domain that binds to a non-CD47 antigen. The additional domain binds to a soluble antigen, an aggregate of antigen, a surface antigen on a cell, a surface antigen on a microbe, or a hapten antigen, wherein the antigen is not CD47.

In some embodiments, the additional non-CD47 binding domain binds to one or more antigens selected from the group consisting of Amyloid Beta, Amyloid fibril, SAP, CD38, SLAMF7, BCMA, GPRC5D, FcRH5, CD138, CD56, CD74, CD26, CD46, CD19, CD20, CD22, CD30, CD33, CD37, CD70, CD117, CD79b, CD123, CD52, CD98, CD205, PD-L1, HER2, EGFR, VEGFR2, VEGFR1, VEGF, CD93, TGFβ, IL-6, IL-6R, IL-8, GDF-15, GFRAL, FGFR2, FGFR3, FGFR4, FGFR1, GD2, GD3, B7-H3, B7-H4, phosphatidylserine, DR5, DR4, DR3, TL1A, CD95 (Fas), TNFR1, TNFR2, CD24, CD31, CD61, CD200, SLAMF3, SLAMF4, HLA-G, HLA-E, HLA-DR, NKG2D ligands (MICA, MICB, ULBP1-6), CD1d-presented lipid antigen, P-Cadherin, N-Cadherin, E-Cadherin, VE-Cadherin, R-cadherin (CDH4), K-cadherin (CDH6), VE-cadherin (CDH5), H-cadherin (CDH13), OB-cadherin, LI-cadherin (CDH17), CDH11, CDH19, CLDN18.2, CLDN6, CLDN3, CLDN4, CLDN7, CLDN1, CLDN2, CLDN5, CLDN8, CLDN9, CLDN10, CLDN11, CLDN12, CLDN13, CLDN14, CLDN15, CLDN16, CLDN17, CLDN20, CLDN23, Connexin 25 (Cx25), Cx26, Cx30, Cx30.3, Cx31, Cx31.1, Cx32, Cx36, C37, Cx43, Cx45, Cx46, Cx50, Nectin-4, Nectin-3, Nectin-2, Nectin-1, Necl-5, Necl-2, Necl-1, Necl-3, Necl-4, CD44, CD44v6, ICAM-1 (CD54), ICAM-2 (CD102), ICAM-3 (CD50), VCAM-1, NCAM, L1CAM, EpCAM, ALCAM (CD66), MCAM, MAdCAM-1, CEACAM5, CEACAM1, CEACAM6, CEACAM4, CEACAM7, CEACAM19, CEACAM3, L-selectin, E-selectin, P-selectin, integrin α581, αvβ1, αvβ3, αvβ5, αvβ6, αvβ8, fibronectin extradomain-β, CTGF, Siglec ligands (sialoglycan), MUC1, MUC1-Tn, MUC5AC, MUC15, MUC16, MUC17, EphA1, EphA2, EphA3, EphA4, EphA5, EphA6, EphA7, EphA8, EphB1, EphB2, EphB3, EphB4, MSLN, CSPG4, CA-IX, DLL3, DLL-4, GPC-3, FRα, PSMA, PSCA, GPNMB, HER3, ROR1, ROR2, Axl, TROP2, EGFRvIII, PTK7, GRP78, IGF-1R, PD-L2, TAG-72, NaPi2b, GUCY2C, LY6G6D, PDGFR-α, c-Met, 5T4, AGS-16, ALK1, ANG-2, SSTR2, ENPP3, gpA33, SLC44A4, SLC34A2, SLC39A6, Notch 1, Notch 2, Notch 3, Notch 4, STEAP1, CCR4, CXCR4, CD71, CD166, DKK-1, LRRC15, FAP, Tenascin, GP75, CD175, CD175s, CD176, Lewis Y, SLeA, MST1R, tissue factor, carbonic anhydrase 6, guanylate cyclase-C, ETBR, LIV-1, RANKL, STRA6, UPK1B, VTCN1, LY6E, Binin-4, Tweak, TweakR, SL TRK6, TEM1, TCRα/β, TCRγ/δ, CD3, 4-1BB, CD28, ICOS, CD27, OX40, GITR, LIGHT, CD226, NKG2D, NKG2C, NKp30, NKp44, NKp46, NKp80, DNAM-1, 2B4, KIR2DS, KIR3DS, NTBA, CD84, Ly9, PD-1, CTLA-4, TIGIT, LAG3, CD73, CD39, TIM3, VISTA, CD96, B7-H3, TMIGD2, BTLA, CD160, CD200R, CCR8, CEACAM1, NKG2A, NKG2B, KIR2DL, KIR3DL, LILRB1, LAIR1, LAIR2, CD16, CD32, CD64, CD89, CD35, CR2, CR3, CR4, CRIg, CD40, CSF1R, SIPRa, PSGL-1, VSIG4, CLEVER-1, LILRA1, LILRA2, LILRA3, LILRA4, LILRA5, LILRA6, LILRB1, LILRB2, LILRB3, LILRB4, LILRB5, LAIR1, LAIR2, CD200R, TREM1, TREM2, CD91, SR-A (CD204), CD36, MARCO, SCARF-1, TIM1, TIM-4, BAI1, Stabilin-2, RAGE, CD300f, CD14, CD44, MerTK, Tyro3, Axl, chemokine receptors, CCR1, CCR2, CCR5, CCR7, CX3CR1, CXCR3, CXCR4, CXCR5, CXCR6, cytokine receptors, IL-2R, IL-15R, IL-7R, TNFα, IL-12R, IFNα, IFNβ, IFNγ, C-type lectin receptors, CD205, CD206, CD280, PLA2R, Dectin-1, Dectin-2, Dectin-3 (MCL/CLECSF8/CLEC4D), CLEC5A, Mincle, DC-SIGN, DC-SIGNR, DNGR-1 (CLEC9A), CLEC2, DCL-1 (CLEC13A), CLEC12B (MAH), MelLec (CLEC1A), LSECtin, LOX-1, BDCA-2, MGL (CLEC10A), CD93, Langerin, M1CL (CLEC12A), DCIR, lipoprotein receptors, CD91, LOX-1, LDLR, Toll-like receptors (TLR), TLR1, TLR2, TLR4, TLR5, TLR6, Siglecs, Siglec-1, Siglec-2, Siglec-3, Siglec-4, Siglec-S, Siglec-6, Siglec-7, Siglec-8, Siglec-9, Siglec-10, Siglec-11, Siglec-12, Siglec-13, Siglec-14, Siglec-15, Siglec-16, DOTA (tetraxetan), DTPA (diethylenetriaminepentaacetic acid), and HSG (histamine-succinyl-glycine).

In some embodiments, the additional non-CD47 binding domain comprises an antigen binding domain of an antibody comprising a set of VH and/or VL CDR sequences that are at least 70%, 75%, 80%, 85%, 90%, or 95% identical to the set of VH and/or VL CDR sequences according to the IMGT numbering scheme from an antibody selected from the group of antibodies as set forth in Table 2 to 4.

In some embodiments, the additional non-CD47 binding domain comprises a peptide or polypeptide selected from the group of peptides and polypeptides as set forth in Table 5.

In some embodiments, the additional non-CD47 binding domain binds to one or more antigens selected from the group consisting of PD-L1, CD38, SLAMF7, CD20, CD19, CD30, CD70, CD117, CA-IX, HER2, EGFR, VEGFR2, VEGFR1, VEGF, CD93, FGFR2, GD2, Claudin 18.2, Claudin 6, Claudin 1, Claudin 2, Claudin 3, Claudin 4, Claudin 7, B7-H3, DLL3, DR5, DR4, CD95, Phosphatidylserine, Nectin-4, CDH3, CDH6, CDH17, CDH2, integrins, CD44, ICAM-1, EpCAM, CEACAM5, CEACAM1, CEACAM6, CD24, HLA-G, FAP, CTGF and TL1A.

In some embodiments, the additional non-CD47 binding domain comprises an antigen binding domain of an antibody comprising a set of VH and/or VL CDR sequences that are at least 70%, 75%, 80%, 85%, 90%, or 95% identical to the set of VH and/or VL CDR sequences according to the IMGT numbering scheme from an antibody selected from the group of antibodies comprising anti-PD-L1 antibody BMS-936559, atezolizumab, durvalumab, avelumab, cosibelimab, sugemalimab, geptanolimab and envafolimab, anti-CD38 antibody daratumumab, isatuximab, SAR442085, felzartamab, mezagitamab, TAK-169, CID-103 and Y150, anti-SLAMF7 antibody elotuzumab and azintuxizumab, anti-CD20 antibody rituximab, ofatumumab, ocrelizumab, ublituximab, and obinutuzumab, anti-CD70 antibody cusatuzumab (ARGX-110), MDX1411, SEA-CD70, vorsetuzumab, IMM40H, IMM40M, anti-CD70 nanobody No. 1, 2 and 3 to 24 (WO2022262100), anti-CD70 nanobody No. 1 to 14 (CN113292652A), anti-CD70 antibodies (U.S. Pat. No. 11,377,500B2), LD70, BR108, MP-0533, and the anti-CD70 antibody moiety of SGN-75, SGN-CD70A, BMS-936561 (MDX-1203), AMG172, ARX-305, PRO-1160, CTX130, ALLO-316, P-CD70-ALLO1, 4SCAR70, C-4-29 and CAT-248, anti-CD117 antibody CDX-0158, CDX-0159 (barzolvolimab), briquilimab (JSP191), eglatoprutug, and MGTA-117, anti-CA-IX antibody girentuximab and BAY 79-4620, anti-HER2 antibody trastuzumab, pertuzumab, and margetuximab, anti-EGFR antibody cetuximab, panitumumab, necitumumab and nimotuzumab, anti-VEGFR2 antibody ramucirumab, alacizumab, olinvacimab, pulocimab and vulinacimab, anti-VEGFR1 antibody icrucumab, anti-VEGF antibody tarcocimab, varisacumab, brolucizumab, abicipar, IMC-1C11, faricimab, vanucizumab, dipacimab, navicixizumab, ivonescimab, anti-FGFR2 antibody bemarituzumab and aprutumab, anti-GD2 antibody dinutuximab and naxitamab, anti-DLL3 antibody rovalpituzumab, tarlatamab and PT217, anti-B7-H3 antibody ifinatamab, mirzotamab and enoblituzumab, anti-DR5 antibody conatumumab, drozitumab, lexatumumab, tigatuzumab, tilogotamab, benufutamab, zaptuzumab, INBRX-109, and IGM-8444, anti-phosphatidylserine antibody bavituximab, anti-claudin 18.2 antibody zolbetuximab, gresonitamab, osemitamab, AB011, PT886 and TJ-CD4B, anti-claudin 6 antibody IMAB027, and TJ-C64B, anti-claudin 4 antibody KM3900, huKM3900, KM3934, 4D3, 5A5, 5D12 and KM3907, anti-claudin 3 antibody ABN501, MORAb-075, KMK3935, IgGH6, h4G3, 5A5 and KM3907, anti-claudin 2 antibody 1A2, anti-claudin 1 antibody 3A2, 7A5, 6F6, OM-7D3-B3 and humanized OM-7D3-B3, anti-CEACAM5 antibody hPRIA3, labetuzumab, cibisatamab, cergutuzumab, tusamitamab, AMG-211, BDC-2034, Clone 5G2, MN-3, MN-15, NEO-201, and 15-1-32, anti-CDH3 antibody PF-03732010, PF-06671008, FF-21101, TSP7 and TSP-S77R, anti-CDH6 antibody DS-6000 and NOV0712, anti-CDH17 antibody ARB202 and BI-905711, anti-integrin antibody volociximab (α5α1), etaracizumab (αvβ3), abciximab and intetumumab, anti-CD44 antibody RG7356, anti-ICAM-1 antibody bersanlimab, enlimomab and VBI-002, anti-EpCAM antibody adecatumumab, edrecolomab, citatuzumab, oportuzumab, solitomab, tucotuzumab and VBI-003, anti-Nectin-4 antibody enfortumab, BA3361, SBT6290 and ETx-22, anti-CD24 antibody hG7-BM3, humanized SWA11 and ATG-031, anti-HLA-G antibody TTX-080, IVS-4001 and JNJ-78306358, anti-FAP antibody sibrotuzumab, simlukafusp alfa, OS4 and MFP5, anti-CTGF antibody pamrevlumab (FG-3019) and anti-TL1A antibody tulisokibart (PRA023), PF-06480605 and TEV-48574.

In some embodiments, the additional non-CD47 binding domain binds to one or more antigens selected from the group consisting of Dectin-1, Dectin-2, CLEC5A, MerTK, TREM1, MARCO, CLEVER-1, PSGL-1, VSIG4, CD40, CD205, CD206, CD36, CD91, DC-SIGN, CLEC9A, TLR5, LILRB1 (ILT2), LILRB2 (ILT4), LILRB4 (ILT3), NKG2D, NKp30, NKp46, NKp80, DNAM-1, PD-1, CTLA-4, TIGIT, LAG3, CD3, 4-1BB, OX40, ICOS, CD27 and CD70.

In some embodiments, the additional non-CD47 binding domain comprises an antigen binding domain of an antibody comprising a set of VH and/or VL CDR sequences that are at least 70%, 75%, 80%, 85%, 90%, or 95% identical to the set of VH and/or VL CDR sequences according to the IMGT numbering scheme from an antibody selected from the group of antibodies comprising anti-Dectin 1 antibody 2M24 and 15E2, anti-Dectin 2 antibody BDC-3042, anti-CLEC5A antibody 1F7, anti-MerTK antibody 18G7 and RGX-019, anti-CD205 antibody 3G9 and OBT076, anti-CD206 antibody CDX-1307, anti-DC-SIGN antibody hDIVI, anti-TREM1 antibody PY159, anti-CLEVER1 antibody bexmarilimab, anti-PSGL-1 antibody VTX-0811, neihulizumab, and leiolizumab, anti-CD36 antibody ONA-0-v1, anti-LILRB1 antibody BND-22, NGM707, AGEN1571, ATG-032 and DM002, anti-LILRB2 antibody MK-4830, JTX-8064, NGM707, IO-108, ES009, ATG-032 and DM002, anti-LILRB4 antibody IO-202, MK-0482, BND-35, NGM831, JTX1484 and SG2901, anti-NKG2D antibody A49,A44 and KYK-2.0, anti-CD3 antibody SP34, UCHT1, mosunetuzumab, tarlatamab, PF-06671008, tebentafusp and TNB-383B, anti-4-1BB antibody utomilumab, AGEN2373, LVGN6051, GEN1046 and TJ-C64B, anti-PD-1 antibody nivolumab and pembrolizumab, anti-CTLA-4 antibody ipilimumab, tremelimumab, botensilimab, nurulimab and BA-3017, and anti-TIGIT antibody tiragolumab, vibostolimab, etigilimab, BGB-A1217 and EOS-448.

In some embodiments, the fusion protein comprises additional non-CD47 binding domains that bind to two non-CD47 antigens, comprising: 1) one antigen selected from the group consisting of Amyloid Beta, Amyloid fibril, SAP, CD38, SLAMF7, BCMA, GPRC5D, FcRH5, CD138, CD56, CD74, CD26, CD46, CD19, CD20, CD22, CD30, CD33, CD37, CD70, CD117, CD79b, CD123, CD52, CD98, CD205, PD-L1, HER2, EGFR, VEGFR2, VEGFR1, VEGF, CD93, TGFβ, IL-6, IL-6R, IL-8, GDF-15, GFRAL, FGFR2, FGFR3, FGFR4, FGFR1, GD2, GD3, B7-H3, B7-H4, phosphatidylserine, DR5, DR4, DR3, TL1A, CD95 (Fas), TNFR1, TNFR2, CD24, CD31, CD61, CD200, SLAMF3, SLAMF4, HLA-G, HLA-E, HLA-DR, NKG2D ligands (MICA, MICB, ULBP1-6), CD1d-presented lipid antigen, P-Cadherin, N-Cadherin, E-Cadherin, VE-Cadherin, R-cadherin (CDH4), K-cadherin (CDH6), VE-cadherin (CDH5), H-cadherin (CDH13), OB-cadherin, LI-cadherin (CDH17), CDH11, CDH19, CLDN18.2, CLDN6, CLDN3, CLDN4, CLDN7, CLDN1, CLDN2, CLDN5, CLDN8, CLDN9, CLDN10, CLDN11, CLDN12, CLDN13, CLDN14, CLDN15, CLDN16, CLDN17, CLDN20, CLDN23, Connexin 25 (Cx25), Cx26, Cx30, Cx30.3, Cx31, Cx31.1, Cx32, Cx36, C37, Cx43, Cx45, Cx46, Cx50, Nectin-4, Nectin-3, Nectin-2, Nectin-1, Necl-5, Necl-2, Necl-1, Necl-3, Necl-4, CD44, CD44v6, ICAM-1 (CD54), ICAM-2 (CD102), ICAM-3 (CD50), VCAM-1, NCAM, L1CAM, EpCAM, ALCAM (CD66), MCAM, MAdCAM-1, CEACAM5, CEACAM1, CEACAM6, CEACAM4, CEACAM7, CEACAM19, CEACAM3, L-selectin, E-selectin, P-selectin, integrin α5B1, αvβ1, αvβ3, αvβ5, αvβ6, αvβ8, fibronectin extradomain-β, CTGF, Siglec ligands (sialoglycan), MUC1, MUC1-Tn, MUC5AC, MUC15, MUC16, MUC17, EphA1, EphA2, EphA3, EphA4, EphA5, EphA6, EphA7, EphA8, EphB1, EphB2, EphB3, EphB4, MSLN, CSPG4, CA-IX, DLL3, DLL-4, GPC-3, FRα, PSMA, PSCA, GPNMB, HER3, ROR1, ROR2, Axl, TROP2, EGFRvIII, PTK7, GRP78, IGF-1R, PD-L2, TAG-72, NaPi2b, GUCY2C, LY6G6D, PDGFR-α, c-Met, 5T4, AGS-16, ALK1, ANG-2, SSTR2, ENPP3, gpA33, SLC44A4, SLC34A2, SLC39A6, Notch 1, Notch 2, Notch 3, Notch 4, STEAP1, CCR4, CXCR4, CD71, CD166, DKK-1, LRRC15, FAP, Tenascin, GP75, CD175, CD175s, CD176, Lewis Y, SLeA, MST1R, tissue factor, carbonic anhydrase 6, guanylate cyclase-C, ETBR, LIV-1, RANKL, STRA6, UPK1B, VTCN1, LY6E, Binin-4, Tweak, TweakR, SLTRK6 and TEM1, and 2) the other antigen selected from the group consisting of TCRα/β, TCRγ/δ, CD3, 4-1BB, CD28, ICOS, CD27, CD70, OX40, GITR, LIGHT, CD226, NKG2D, NKG2C, NKp30, NKp44, NKp46, NKp80, DNAM-1, 2B4, KIR2DS, KIR3DS, NTBA, CD84, Ly9, PD-1, CTLA-4, TIGIT, LAG3, CD73, CD39, TIM3, VISTA, CD96, B7-H3, TMIGD2, BTLA, CD160, CD200R, CCR8, CEACAM1, NKG2A, NKG2B, KIR2DL, KIR3DL, LILRB1, LAIR1, LAIR2, CD16, CD32, CD64, CD89, CD35, CR2, CR3, CR4, CRIg, CD40, CSF1R, SIPRa, PSGL-1, VSIG4, CLEVER-1, LILRA1, LILRA2, LILRA3, LILRA4, LILRA5, LILRA6, LILRB1, LILRB2, LILRB3, LILRB4, LILRB5, LAIR1, LAIR2, CD200R, TREM1, TREM2, CD91, SR-A (CD204), CD36, MARCO, SCARF-1, TIM1, TIM-4, BAI1, Stabilin-2, RAGE, CD300f, CD14, CD44, MerTK, Tyro3, Axl, chemokine receptors, CCR1, CCR2, CCR5, CCR7, CX3CR1, CXCR3, CXCR4, CXCR5, CXCR6, cytokine receptors, IL-2R, IL-15R, IL-7R, TNFα, IL-12R, IFNα, IFNβ, IFNγ, C-type lectin receptors, CD205, CD206, CD280, PLA2R, Dectin-1, Dectin-2, Dectin-3 (MCL/CLECSF8/CLEC4D), CLEC5A, Mincle, DC-SIGN, DC-SIGNR, DNGR-1 (CLEC9A), CLEC2, DCL-1 (CLEC13A), CLEC12B (MAH), MelLec (CLEC1A), LSECtin, LOX-1, BDCA-2, MGL (CLEC10A), CD93, Langerin, M1CL (CLEC12A), DCIR, lipoprotein receptors, CD91, LOX-1, LDLR, Toll-like receptors (TLR), TLR1, TLR2, TLR4, TLR5, TLR6, Siglecs, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, DOTA (tetraxetan), DTPA (diethylenetriaminepentaacetic acid), and HSG (histamine-succinyl-glycine).

In one aspect, the present disclosure provides a Fe fusion protein comprising: 1) a homodimeric Fc comprising two Fc chains, and 2) a SIRP IgV domain comprising one, two, three, four or more said SIRP IgV monomers, wherein the SIRP IgV domain is linked to the N-terminal and/or C-terminal of the Fc chain. In some embodiments, the one, two, three or more said SIRP IgV monomers of the SIRP IgV domain comprise the same or different amino acid sequence selected from the group consisting of SEQ ID NO: 3 to 81. In some embodiments, the SIRP IgV domain comprises an amino acid sequence selected from the group consisting of SEQ ID NO: 3 to 113.

In one aspect, the present disclosure provides an antibody fusion protein comprising: 1) a partial heavy chain comprising CH1-hinge-CH2-CH3 from the N-terminal to C-terminal, 2) a partial light chain comprising CL domain, wherein the CL domain pairs with the CH1 domain of the partial heavy chain with disulfide bond, 3) the Fc region of the two partial heavy chains form a homodimeric Fe, and 4) a SIRP IgV domain comprising one, two, three or more said SIRP IgV monomers, wherein the SIRP IgV domain is linked preferably through a linker to the N-terminal of the partial light chain and/or partial heavy chain. In some embodiments, the one, two, three or more said SIRP IgV monomers of the SIRP IgV domain comprise the same or different amino acid sequence selected from the group consisting of SEQ ID NO: 3 to 81. In some embodiments, the SIRP IgV domain comprises an amino acid sequence selected from the group consisting of SEQ ID NO: 3 to 113.

In one aspect, the present disclosure provides a Fc fusion protein comprising: 1) a first polypeptide comprising a first SIRP IgV domain and a first Fe region, wherein the first SIRP IgV domain comprises one, two, three, four or more of the said SIRP IgV monomers, and is linked to the N-terminal of the first Fc region, and 2) a second polypeptide comprising a second SIRP IgV domain and a second Fc region, wherein the second SIRP IgV domain comprises one, two, three, four or more of the said SIRP IgV monomers, and is linked to the N-terminal of the second Fc region, wherein the Fe region of the two polypeptides form a heterodimeric Fc, and the first and second SIRP IgV domains are not the same. In some embodiments, the one, two, three or more said SIRP IgV monomers of the first and second SIRP IgV domain comprise different amino acid sequence selected from the group consisting of SEQ ID NO: 3 to 81. In some embodiments, the first and second SIRP IgV domains comprise a different amino acid sequence selected from the group consisting of SEQ ID NO: 3 to 113.

In one aspect, the present disclosure provides a Fe fusion protein comprising: 1) a homodimeric Fe comprising two Fc chains, 2) a SIRP IgV domain comprising one, two, three, four or more said SIRP IgV monomers, wherein the SIRP IgV domain is linked to the N-terminal of the Fc chain, and 3) a single-chain binding domain that binds to a non-CD47 antigen, wherein the single-chain binding domain is linked preferably through a linker to the C-terminal of the Fc chain. In some embodiments, the one, two, three or more said SIRP IgV monomers of the SIRP IgV domain comprise the same or different amino acid sequence selected from the group consisting of SEQ ID NO: 3 to 81. In some embodiments, the SIRP IgV domain comprises an amino acid sequence selected from the group consisting of SEQ ID NO: 3 to 113. In some embodiments, the single-chain binding domain comprises an amino acid sequence selected from the group consisting of SEQ ID NO: 215 to 253.

In one aspect, the present disclosure provides an antibody fusion protein comprising: 1) a full antibody binding to a non-CD47 antigen comprising two heavy chains and two light chains, wherein the two Fc region of the two heavy chains form a homodimeric Fc, and 2) a SIRP IgV domain, wherein the SIRP IgV domain comprises one, two, three or more said SIRP IgV monomers, and is linked preferably through a linker to the N-terminal of the heavy chain or the light chain of the antibody. In some embodiments, the one, two, three or more said SIRP IgV monomers of the SIRP IgV domain comprise the same or different amino acid sequence selected from the group consisting of SEQ ID NO: 3 to 81. In some embodiments, the SIRP IgV domain comprises an amino acid sequence selected from the group consisting of SEQ ID NO: 3 to 113. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain of the antibody fusion protein.

In one aspect, the present disclosure provides an antibody fusion protein comprising: 1) a full antibody binding to a non-CD47 antigen comprising two heavy chains and two light chains, wherein the two Fc region of the two heavy chains form a homodimeric Fc, and 2) a SIRP IgV domain, wherein the SIRP IgV domain comprises one, two, three or more said SIRP IgV monomers of SIRPα, SIRPβ and/or SIRPγ, and is linked preferably through a linker to the N-terminal of the heavy chain and the light chain of the antibody. In some embodiments, the one, two, three or more said SIRP IgV monomers of the SIRP IgV domain comprise the same or different amino acid sequence selected from the group consisting of SEQ ID NO: 3 to 81. In some embodiments, the SIRP IgV domain comprises an amino acid sequence selected from the group consisting of SEQ ID NO: 3 to 113. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain of the antibody fusion protein,

In one aspect, the present disclosure provides an antibody fusion protein comprising: 1) a first polypeptide comprising a SIRP IgV domain and a Fc region, wherein the SIRP IgV domain comprises one, two, three, four or more of the said SIRP IgV monomers, and is linked to the N-terminal of the Fe region, 2) a full heavy chain of an antibody and a full light chain of an antibody, wherein the antibody heavy chain and the light chain pair to form an half-antibody that binds to a non-CD47 antigen, and 3) the Fe region of the first polypeptide and the antibody heavy chain form a heterodimeric Fc. In some embodiments, the one, two, three, four or more said SIRP IgV monomers of the SIRP IgV domain of the first polypeptide comprise the same or different amino acid sequence selected from the group consisting of SEQ ID NO: 3 to 81. In some embodiments, the SIRP IgV domain of the first polypeptide comprises an amino acid sequence selected from the group consisting of SEQ ID NO: 3 to 113. In some embodiments, the antibody fusion protein comprises a third antigen-binding domain, wherein the third antigen-binding domain is a single-chain polypeptide binding to a third antigen and is linked through a linker to the C-terminal of the Fc chains or light chain of the antibody fusion protein,

In one aspect, the present disclosure provides an antibody fusion protein comprising: 1) a first polypeptide comprising a SIRP IgV domain and a Fc region, wherein the SIRP IgV domain comprises two, three, four or more SIRP IgV monomers of SIRPα, and is linked to the N-terminal of the Fe region, 2) a full heavy chain of an antibody and a full light chain of an antibody, wherein the antibody heavy chain and the light chain pair to form an half-antibody that binds to a non-CD47 antigen, and 3) the Fc region of the first polypeptide and the antibody heavy chain form a heterodimeric Fc. In some embodiments, the two, three, four or more SIRPα IgV monomers of the SIRP IgV domain comprise the same or different amino acid sequence selected from the group consisting of SEQ ID NO: 3 to 11, 22 to 30, 34 to 42, 46 to 54, 58 to 66, and 70 to 78. In some embodiments, the SIRP IgV domain comprises an amino acid sequence selected from the group consisting of SEQ ID NO: 82 to 91, 97 to 102, and 110 to 113.

In one aspect, the present disclosure provides an antibody fusion protein comprising: 1) a first polypeptide comprising a SIRP IgV domain and a Fc region, wherein the SIRP IgV domain comprises one, two, three, four or more SIRP IgV monomers of SIRPβ and/or SIRPγ, and is linked to the N-terminal of the Fc region, 2) a full heavy chain of an antibody and a full light chain of an antibody, wherein the antibody heavy chain and the light chain pair to form an half-antibody that binds to a non-CD47 antigen, and 3) the Fe region of the first polypeptide and the antibody heavy chain form a heterodimeric Fc. In some embodiments, the one, two, three, four or more SIRPβ and/or SIRPγ IgV monomers of the SIRP IgV domain comprise the same or different amino acid sequence selected from the group consisting of SEQ ID NO: 12 to 21, 31 to 33, 43 to 45, 55 to 57, 67 to 69, and 79 to 81. In some embodiments, the SIRP IgV domain of the first polypeptide comprises an amino acid sequence selected from the group consisting of SEQ ID NO: 12 to 21, 31 to 33, 43 to 45, 55 to 57, 67 to 69, 79 to 81, 92 to 96, and 103 to 109.

In one aspect, the present disclosure provides an antibody fusion protein comprising: 1) a first polypeptide comprising a first SIRP IgV domain and a Fc region, wherein the SIRP IgV domain comprises one, two, three or more said SIRP IgV monomers of SIRPα, SIRPβ and/or SIRPγ and is linked to the N-terminal of the Fc region, 2) a full heavy chain of an antibody and a full light chain of an antibody, wherein the antibody heavy chain and the light chain pair to form an half-antibody that binds to a non-CD47 antigen, 3) a second SIRP IgV domain, wherein the second SIRP IgV domain comprises one, two, three or more said SIRP IgV monomers of SIRPα, SIRPβ and/or SIRPγ and is linked preferably through a linker to the N-terminal of the antibody heavy chain and/or light chain, and 4) the Fc region of the first polypeptide and the antibody heavy chain form a heterodimeric Fc. In some embodiments, the first and second SIRP IgV domains comprise the same or different SIRP IgV monomers. In some embodiments, the first and second SIRP IgV domains comprise the same or different amino acid sequene selected from the group consisting of SEQ ID NO: 3 to 113. In some embodiments, the first SIRP IgV domain comprise an amino acid sequene selected from the group consisting of SEQ ID NO: 3 to 21 and the second SIRP IgV domain comprises one IgV extracelluar domain of SIRPα, SIRPβ or SIRPγ comrpising a mutation selected from the group consisting of K53H+K68H, R69H+K68H, K53H+Q52H, R69H+Q52H, R69H+Q37H/L37H, K53H+Q37H/L37H, R69H, K53H, R69H+I31E, K53H+I31E, K96H, V33H, and P35H. In some embodiments, the antibody fusion protein comprises additionally a third antigen-binding domain, wherein the third antigen-binding domain is a single-chain polypeptide binding to a third antigen and is linked through a linker to the C-terminal of the Fc chains or light chain of the antibody fusion protein,

In one aspect, the present disclosure provides an antibody fusion protein comprising: 1) a full antibody binding to a non-CD47 antigen comprising two heavy chains comprising heterodimeric Fe region, wherein the Fc region of the two heavy chains form a heterodimeric Fc. 2) a common light chain, wherein the light chain pairs with each of the two heavy chains to form two Fab domains, and 3) a SIRP IgV domain, wherein the SIRP IgV domain is linked preferably through a linker to the N-terminal of one of the two heterodimeric heavy chains. In some embodiments, the SIRP IgV domain comprises one, two, three or more SIRP IgV monomers comprising the same or different amino acid sequence selected from the group consisting of SEQ ID NO: 3 to 81. In some embodiments, the SIRP IgV domain comprises one SIRP IgV domain comprising an amino acid sequence selected from the group consisting of SEQ ID NO: 3 to 113. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain of the antibody fusion protein,

In one aspect, the present disclosure provides an antibody fusion protein comprising: 1) an antibody binding to a non-CD47 antigen comprising a first heavy chain comprising VH-CH1-CH2-CH3 and a second chimeric heavy chain comprising VL-CL-CH2-CH3 from the N-terminal to C-terminal, wherein the two Fc region of the two heavy chains form a heterodimeric Fc, 2) a light chain comprising VL-CL from the N-terminal to C-terminal, wherein the light chain pairs with the first heavy chain to form the first Fab domain of the antibody, 3) a Fd chain comprising VH-CH1, wherein the Fd chain pairs with the second chimeric heavy chain to form the second Fab domain of the antibody, and 4) a SIRP IgV domain, wherein the SIRP IgV domain is linked preferably through a linker to the N-terminal of the light chain (VL-CL) or the Fd chain (VH-CH1) or one of the two heterodimeric heavy chains. In some embodiments, the SIRP IgV domain comprises one, two, three or more SIRP IgV monomers comprising the same or different amino acid sequence selected from the group consisting of SEQ ID NO: 3 to 81. In some embodiments, the SIRP IgV domain comprises an amino acid sequence selected from the group consisting of SEQ ID NO: 3 to 113. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain and Fd chain of the antibody fusion protein,

In one aspect, the present disclosure provides an antibody fusion protein comprising: 1) an antibody binding to a non-CD47 antigen comprising a first heavy chain comprising VH-CH1-CH2-CH3 and a second chimeric heavy chain comprising VH-CL-CH2-CH3 from the N-terminal to C-terminal, wherein the two Fc region of the two heavy chains form a heterodimeric Fc, 2) a light chain comprising VL-CL from the N-terminal to C-terminal, wherein the light chain pairs with the first heavy chain to form the first Fab domain of the antibody, 3) a chimeric light chain comprising VL-CH1, wherein the chimeric light chain pairs with the second chimeric heavy chain to form the second Fab domain of the antibody, and 4) a SIRP IgV domain, wherein the SIRP IgV domain is linked preferably through a linker to the N-terminal of the light chain (VL-CL) or the chimeric light chain (VL-CH1) or one of the two heterodimeric heavy chains. In some embodiments, the SIRP IgV domain comprises one, two, three or more SIRP IgV monomers comprising the same or different amino acid sequence selected from the group consisting of SEQ ID NO: 3 to 81. In some embodiments, the SIRP IgV domain comprises an amino acid sequence selected from the group consisting of SEQ ID NO: 3 to 113. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain of the antibody fusion protein.

In one aspect, the present disclosure provides an antibody fusion protein comprising: 1) a first polypeptide comprising a Fc region and a SIRP IgV domain comprising one, two, three, four or more SIRP IgV monomers, wherein the SIRP IgV domain is directly linked to the N-terminal of the Fc region; 2) a chimeric heavy chain comprising from N-terminal to C-terminal VH2-CL-linker-VH1-CH1-Hinge-CH2-CH3, wherein the chimeric heavy chain forms a heterodimeric Fc with the Fc region of the first polypeptide; 3) a first light chain comprising VL1-CL, wherein the light chain pairs with the VH1-CH1 part of the chimeric heavy chain to form the first Fab domain; 4) a second chimeric light chain comprising VL2-CH1, wherein the chimeric light chain pairs with the VH2-CL part of the chimeric heavy chain to form the 2^{nd} Fab domain; and 5) the first and second Fab domains bind to two different non-CD47 antigens. In certain embodiments, the SIRP IgV monomers of the SIRP IgV domain comprise SIRPα, SIRPβ and/or SIRPγ IgV monomers. In certain embodiments, the SIRP IgV monomers of the SIRP IgV domain comprise the same or different amino acid sequence selected from the group consisting of SEQ ID NO: 3 to 81. In certain embodiments, the SIRP IgV monomers of the SIRP IgV domain comprise SIRPβ and/or SIRPγ IgV monomers, comprising an amino acid sequence selected from the group consisting of SEQ ID NO: 12 to 21, 31 to 33, 43 to 45, 55 to 57, 67 to 69, and 79 to 81. In certain embodiments, the SIRP IgV domain comprises an amino acid sequence selected from the group consisting of SEQ ID NO: 3 to 113. In certain embodiments, the linker of the chimeric heavy chain comprises an amino acid sequence of GGGGSGGGGS. In certain embodiments, the first Fab domain binds to a surface receptor of an effector cell and the second Fab domain binds to an antigen associated with a diseased cell or tissue, or vice versa. In certain embodiments, the first Fab domain binds to a cell adhesion molecule and the second Fab domain binds to an antigen associated with a diseased cell or tissue, or vice versa.

In one aspect, the present disclosure provides an antibody fusion protein comprising: 1) a first heavy chain comprising VH1-CH1-hinge-CH2-CH3 and a first light chain comprising VL1-CL, wherein the first heavy chain and light chain pair to form the first Fab domain; 2) a second chimeric heavy chain comprising from N-terminal to C-terminal VH2-CL-Hinge-CH2-CH3, and a second chimeric light chain comprising VL2-CH1, wherein the chimeric light chain pairs with the VH2-CL part of the chimeric heavy chain to form the 2^{nd} Fab domain; 3) wherein the first heavy chain and the chimeric heavy chain form a heterodimeric Fc; 4) a SIRP IgV domain comprising one, two, three or four SIRP IgV monomers, wherein the SIRP IgV domain is linked preferably through a linker to the N-terminal of the light chain, chimeric light chain, heavy chain and/or chimeric heavy chain; and 5) wherein the first and second Fab domains bind to two different non-CD47 antigens. In certain embodiments, the SIRP IgV monomers of the SIRP IgV domain comprise SIRPα, SIRPβ and/or SIRPγ IgV monomers. In certain embodiments, the SIRP IgV monomers of the SIRP IgV domain comprise the same or different amino acid sequence selected from the group consisting of SEQ ID NO: 3 to 81. In certain embodiments, the SIRP IgV monomers of the SIRP IgV domain comprise SIRPβ and/or SIRPγ IgV monomers, comprising an amino acid sequence selected from the group consisting of SEQ ID NO: 12 to 21, 31 to 33, 43 to 45, 55 to 57, 67 to 69, and 79 to 81. In certain embodiments, the SIRP IgV domain comprises an amino acid sequence selected from the group consisting of SEQ ID NO: 3 to 113. In certain embodiments, the first Fab domain binds to a surface receptor of an effector cell and the second Fab domain binds to an antigen associated with a diseased cell or tissue, or vice versa. In certain embodiments, the first Fab domain binds to a cell adhesion molecule and the second Fab domain binds to an antigen associated with a diseased cell or tissue, or vice versa.

In one aspect, the present disclosure provides a Fe fusion protein comprising: 1) a first polypeptide comprising a first SIRP IgV domain and a Fc region, wherein the SIRP IgV domain comprises one, two, three, four or more of the said SIRP IgV monomers, and is linked to the N-terminal of the Fc region, 2) a second polypeptide comprising a sing-chain binding domain that binds to a non-CD47 antigen and a Fe region, wherein the single-chain binding domain is linked preferably through a linker to the N-terminal of the Fc region, 3) optionally a second SIRP IgV domain, wherein the optional second SIRP IgV domain comprises one, two, three or more said SIRP IgV monomers and is linked preferably through a linker to the N-terminal of the single-chain binding domain of the second polypeptide, and 4) the Fc region of the first and second polypeptides form a heterodimeric Fc. In some embodiments, the one, two, three, four or more said SIRP IgV monomers of the first SIRP IgV domain of the first polypeptide and the optional second SIRP IgV domain of the second polypeptide comprise the same or different amino acid sequence selected from the group consisting of SEQ ID NO: 3 to 81. In some embodiments, the first SIRP IgV domain of the first polypeptide and the optional second SIRP IgV domain of the second polypeptide comprise the same or different amino acid sequence selected from the group consisting of SEQ ID NO: 3 to 113. In some embodiments, the single-chain binding domain of the second polypeptide comprises an amino acid sequence selected from the group consisting of SEQ ID NO: 221 to 241. In some embodiments, the antibody fusion protein comprises additionally a third antigen-binding domain, wherein the third antigen-binding domain is a single-chain polypeptide binding to a third antigen and is linked through a linker to the C-terminal of the Fc chain of the Fe fusion protein.

In some embodiments, the homodimeric Fe comprises an amino acid sequence selected from the group consisting of SEQ ID NO: 130 to 135. In some embodiments, the heterodimeric Fe comprises a pair of Fe amino acid sequence selected from the group consisting of SEQ ID NO: 136:137, 138:139, 140:141 and 142:143, wherein the “:” indicates pairing of the two Fe sequences from the left to right of the “:” symbol. In some embodiments, the homodimeric or heterodimeric Fc comprises a human IgG1 Fe with wild type or enhanced effector function.

In some embodiments, the third antigen-binding domain of the antibody fusion protein or Fe fusion protein binds to an antigen selected from the group consisting of TGFβ, VEGF, CTGF, TL1A, GDF15, IL-8, IL-6, Dectin-1, CLEC5A, MerTK, CD205, CD206, CD91, ILT2, ILT4, TLR5, NKG2D, NKp46, NKp30, CD28, ICOS, NKG2D ligands, Siglec ligands (sialoglycan), CD70, CD24, HLA-G, cadherins, claudins, nectins, integrin, and FGFR. In certain embodiments, the third antigen-binding domain comprises an amino acid sequence selected from the group consisting of SEQ ID NO: 215 to 241.

In one aspect, the present disclosure provides a Fab fusion protein comprising: 1) a Fab domain comprising a Fd chain comprising VH-CH1 and a light chain comprising VL-CL from the N-terminal to C-terminal, wherein the Fab domain binds to a non-CD47 antigen and 2) a SIRP IgV domain, wherein the SIRP IgV domain comprises one, two, three, four or more said SIRP IgV extracellular domain monomers of SIRPα, SIRPβ and/or SIRPγ, and is linked to the N-terminal and/or C-terminal of the Fd chain and/or light chain of the Fab domain. In some embodiments, the one, two, three, four or more said SIRP IgV monomers of the SIRP IgV domain comprise the same or different amino acid sequence selected from the group consisting of SEQ ID NO: 3 to 81. In some embodiments, the SIRP IgV domain comprises an amino acid sequence selected from the group consisting of SEQ ID NO: 3 to 113. In some embodiments, the Fab fusion protein comprises additionally a third single-chain antigen binding domain that binds to a second non-CD47 antigen, wherein the third antigen binding domain and the SIRP IgV domain are separately linked preferably through a linker to a different chain (Fd chain or light chain) of the Fab domain, at the N-terminal or C-terminal of the chain.

In some embodiments, the linker comprises an amino acid sequence selected from the group consisting of SEQ ID NO: 118 to 123, preferably comprising SEQ ID NO: 121.

### Protein Drug Conjugate

In one aspect, the present disclosure provides a protein drug conjugate comprising a SIRP IgV monomer of present disclosure, a SIRP IgV multimer polypeptide of present disclosure comprising two or more SIRP IgV monomers, a fusion polypeptide of present disclosure comprising a CD47-binding SIRP IgV domain and a non-CD47 binding domain, or a Fc fusion protein, an antibody fusion protein, a Fab fusion protein or other fusion protein of present disclosure comprising one or more of the fusion polypeptides of present disclosure.

In some embodiments, the protein conjugate comprises a SIRP IgV monomer, wherein the SIRP IgV monomer comprises an amino acid sequence selected from the group consisting of SEQ ID NO: 3 to 81. In some embodiments, the protein conjugate comprises a SIRP IgV monomer, wherein the SIRP IgV monomer comprises a mutation of K53H, R69H, Q52H, K68H, Q52H+K68H, I31E/L31E or I31D/L31D. In some embodiments, the protein conjugate comprises a SIRP IgV multimer polypeptide comprising two, three or four SIRP IgV monomers, wherein the SIRP IgV monomer comprises a substitution selected from the group consisting of K53H, R69H, Q52H, K68H, I31E/L31E, I31D/L31D, Q52H+K68H, K53H+K68H, R69H+K68H, K53H+Q52H, R69H+Q52H, K53H+K68H+Q52H, R69H+K68H+Q52H, K53H+I31E/L31E, R69H+I31E/L31E, K68H+I31E/L31E, Q52H+I31E/L31E, Q52H+K68H+I31E/L31E, K53H+I31D/L31D, R69H+I31D/L31D, K68H+I31D/L31D, Q52H+I31D/L31D, and Q52H+K68H+I31D/L31D,

In some embodiments, the protein drug conjugate comprises at least one conjugated moiety selected from the group consisting of a cytotoxic agent, a cytostatic agent, a radioactive isotope or compound, a chelator, a calreticulin-inducing agent, a PP1/GADD34 inhibitor, an immune stimulatory adjuvant moiety, a steroid, an immunosuppressor, DNA, RNA, a photosensitizer, a toxin, and an enzyme/pro-drug converting enzyme.

In some embodiments, the conjugated moiety comprises a cytotoxic agent selected from the group consisting of an auristatins, a topoisomerase inhibitor, a maytansinoids, a tubulysins, a taxane, a trichothecene, a vinca alkaloids, methotrexate, a camptothecin, an etoposide, a calicheamicin, an anthracycline, a duocarmycin, a benzodiazepine, an amatoxin, thailanstatin A and a spliceostatin.

In some embodiments, the conjugated moiety comprises a cytotoxic agent selected from the group consisting of SN-38, Dxd, exatecan, MMAE, MMAF, DM1, DM4, eribulin, seco-DUBA, PBD, adriamycin, doxorubicin, daunorubicin, epirubicin, idarubicin, PNU-159682, tautomycin, calyculin A, salubrinal,

In some embodiments, the conjugated moiety comprises a cytotoxic agent selected from the group consisting of tubulin inhibitors, DNA topoisomerase inhibitors, DNA minor groove binders, DNA alkylating agents, DNA intercalating agents, RNA polymerase inhibitors, spliceosome inhibitors or nicotinamide phosphoribosyltransferase inhibitors (NAMPTi).

In some embodiments, the conjugated moiety comprises a radioactive isotope or compound selected from the group consisting of ²²⁵Ac, ²¹¹At, ²¹²Bi, ²²⁴Ra, ²²³Ra, ²²⁷Th, ¹⁴C, ⁶²Cu, ⁶⁴Cu, ⁶⁴Cu, ¹⁸F, ⁵⁶Ga, ⁶⁶Ga, ⁶⁸Ga, ¹²³I, ¹²⁵I, ¹³¹I, ¹¹¹In, ¹⁷⁷Lu, ¹⁵O, ²¹²Pb, ¹⁸⁶Re, ¹⁸⁸Re, ⁴⁴⁵c, ¹⁴⁹Tb, ¹⁵²Tb, ¹⁶¹Tb. ⁹⁹Y or ⁸⁹Zr.

In some embodiments, the conjugated moiety comprises a chelator. In certain embodiments, the chelator preferentially comprises DOTA, DOTATATE, or DOTA-Bn. In additional embodiments, the foregoing chelator optionally chelates with ¹⁷⁷Lu.

In some embodiments, the conjugated moiety comprises a calreticulin-inducing agent selected from the group consisting of anthracyclin such as doxorubicin, doxorubicin, daunorubicin, epirubicin, idarubicin and mitoxantrone, and a PP1/GADD34 inhibitor such as tautomycin, calyculin A and salubrinal, or fullerenols.

In some embodiments, the conjugated moiety comprises an agonist to a pattern recognition receptor (PRR) for pathogen-associated molecular patterns (PAMPs) or damage-associated molecular patterns (DAMPs). In certain embodiments, the foregoing pattern recognition receptors include but are not limited to, Toll-like receptors (TLRs), STimulator of INterferon Genes (STING), C-type lectin receptors (CLRs), Rig-I-like receptors (RLRs) and NOD-like receptors (NLRs), as described in reference such as (Li et al., Signal Transduct Target Ther, 2021).

In some embodiments, the conjugated moiety comprises an agonist to TLR3, TLR7, TLR8, TLR9, STING, and/or RIG-1.

In some embodiments, the conjugated moiety comprises a TLR7 agonist selected from the group consisting of imiquimod, gardiquimod, loxoribine, GSK2245035, 852A, GS-9620, RO6864018, RO7020531, CL264, CL307, 852A, BNT411, DSP-0509, LHC165, NJH395, RO7119929 and TQ-A3334, or a TLR8 agonist selected from the group consisting of IRM1, IRM2, IRM3, TL8-506 and the TLR8 agonist moiety of SBT6050, or a TLR7/8 dual agonist selected from the group consisting of resiquimod, MEDI9197, T785, BDB001, BDB018, BDB030, CV8102, NKTR-262, CL097, CL075 and the TLR7/8 agonist moiety of BDC-1001, or a TLR9 agonist selected from the group consisting of MGN1703, SD-101, IMO-2125, CpG-7909, CYT003, DUK-CpG-001, GNKG168, EMD1202081, CpG10104, AZD1419 and the TLR9 agonist moiety of the antibody-drug conjugate TAC-001 and ALTA-002, or a STING agonist selected from the group consisting of ALG-031048, E7766, JNJ-6196, MK-2118, MSA-1, MSA-2, SNX281, SR-717, TAK676, TTI-10001, and the STING agonist moiety of the antibody-drug conjugate XMT-2056 and CRD-5500, or a RIG-1 agonist selected from the group consisting of MK-4621 (RGT100), SLR14, SLR20, KIN700, KIN1148, KIN600, KIN500, KIN100, KIN101, KIN400, KIN2000, and SB-9200.

In some embodiments, the conjugated moiety comprises an agonist for TLR7 and/or TLR8 that comprises the TLR7/8 agonist moiety of BDC-1001 or the TLR8 agonist moiety of SBT6050, or a TLR9 agonist that comprises the TLR9 agonist moiety of the antibody-drug conjugate TAC-001 or ALTA-002, or a STING agonist that comprises the STING agonist moiety of the antibody-drug conjugate XMT-2056 or CRD-5500.

In certain embodiments, the conjugated moiety comprises photosensitive agents including but not limiting to silicon phthalocyanine dye such as IRDye700DX, that are known in the arts, for example, as described in patent U.S. Pat. No. 8,524,239B2 and the reference (Maczynska et al., Cell Death Dis, 2020). In certain embodiments, the conjugated moiety comprises a protein toxin, or an enzymatically active toxin of bacterial, fungal, plant, or animal origin, or fragments thereof. In certain embodiments, the conjugated moiety comprises an enzymes/pro-drug converting enzyme.

Techniques for conjugating various types of therapeutic agents (e.g. cytotoxic or cytostatic small molecules, TLR agonists, STING agonists, radioactive compounds or chelators, DNA, RNA, photosensitizer, toxins and enzymes) to proteins, especially to antibodies, are well known in the arts.

In some embodiments, the conjugated moiety is covalently conjugated to cystein, lysine, carbohydrate glyco-group or other chemically active group of the protein or Fe through techniques known in the arts.

In some embodiments, a method of treating a CD47-expressing disease in a mammal comprises administering an effective amount of a SIRP IgV monomer of present disclosure, a SIRP IgV multimer polypeptide of present disclosure, a fusion polypeptide of present disclosure, a fusion protein of present disclosure, and/or a protein drug conjugate of present disclosure, to a mammal in need thereof. In some embodiments, a method of treating a CD47-expressing disease in a mammal comprising administrating an effective amount of a protein or protein conjugate comprising a CD47-binding SIRP IgV domain, wherein the protein or protein conjugate avoids or reduces platelet binding and/or thrombocytopenia, wherein the SIRP IgV domain of the protein or protein conjugate comprises a substitution of K53H, R69H, Q52H and/or K68H. In some embodiments, a method of treating a CD47-expressing disease in a mammal comprising administrating an effective amount of a T cell engager comprising a SIRP IgV domain binding to CD47 and a CD3 binding domain that binds to and activate T cells, wherein the CD47-binding T cell engager avoids or reduces fratricide of CD47-positive T cells at physiological pH, wherein the SIRP IgV domain of the T cell engager comprises a mutation of K53H, R69H, Q52H and/or K68H. In some embodiments, the disease is a disease of cancer, fibrosis, atherosclerosis, inflammation or senescence. In certain embodiments, the disease is a disease of cancer. In certain embodiments of the method, the mammal is a human.

### Synthetic Receptor for Cell Therapy

In one aspect, the present disclosure provides a synthetic receptor for engineered cell therapy comprising a SIRP IgV monomer of present disclosure, a SIRP IgV multimer polypeptide of present disclosure comprising two or more SIRP IgV monomers, or a fusion polypeptide of present disclosure comprising a CD47-binding SIRP IgV domain and a non-CD47 binding domain. In some embodiments, the synthetic receptor comprises a chimeric antigen receptor (CAR), a synthetic T cell receptor (TCR), or a T cell-antigen coupler (TAC), wherein the synthetic receptor comprises an antigen binding domain comprising a SIRP IgV monomer, a SIRP IgV multimer polypeptide, or a SIRP IgV domain-comprising fusion polypeptide of present disclosure, a transmembrane domain and an intracellular signaling domain.

In some embodiments, the synthetic receptor comprises a SIRP IgV monomer, wherein the SIRP IgV monomer comprises an amino acid sequence selected from the group consisting of SEQ ID NO: 3 to 81. In some embodiments, the synthetic receptor comprises a SIRP IgV monomer, wherein the SIRP IgV monomer comprises a mutation of K53H, R69H, Q52H, K68H, I31E/L31E, I31D/L31D, Q52H+K68H, K53H+I31E, R69H+I31E, K68H+I31E, K53H+I31D, R69H+I31D or K68H+I31D.

In some embodiments, the synthetic receptor is a chimeric antigen receptor (CAR) comprising from the N-terminal to C-terminal an antigen binding domain comprising a SIRP IgV domain, an extracellular spacer domain, a transmembrane domain, a co-stimulatory domain and and an intracellular signaling domain.

In some embodiments, the synthetic receptor is a T cell receptor (TCR) fusion protein comprising a SIRP IgV domain, wherein the SIRP IgV domain is linked directly or through a linker to the N-terminal of a TCR subunit and wherein the TCR fusion protein incorporates into a TCR when expressed in a T cell. In certain embodiments, the TCR subunit is selected from the group consisting of CD3ε, CD3γ, CD3δ, TCRα, TCRβ, TCRγ and TCRδ. In one embodiment, the TCR subunit is preferentially CD3ε, wherein the T cell receptor complex comprises two CD3ε units.

In some embodiments, the synthetic receptor is a T cell receptor (TCR) fusion protein, comprising an antigen binding domain, wherein the antigen binding domain is linked to the N-terminal of the constant domain of both partial TCRα and TCRβ or both partial TCRγ and TCRδ, and wherein the TCRα and TCRβ fusion protein or the TCRγ and TCRδ fusion protein incorporates into a TCR when expressed in a T cell.

In some embodiments the synthetic receptor is a T cell antigen coupler (TAC), comprising from the N-terminal to C-terminal an antigen binding domain, a second domain binding to a protein associated with the T cell receptor complex and a third domain comprising a T cell receptor signaling domain.

In some embodiments, the antigen binding domain comprises an additional non-CD47 binding domain linked to the C-terminal of the SIRP IgV domain.

In some embodiments, the additional domain of the antigen binding domain binds to an antigen selected from the group consisting of CD19, CD20, CD22, CD37, BCMA, GPRC5D, CD70, CD117, NKG2D ligands, ROR1, MSLN, claudin 18.2, claudin 6, GPC3, HER2, GUCY2C, PAP, TSHR, ALPP, GPC3, EGFR-VIII, GD2, DLL3, IL13Ra2, PSMA, PSCA, MUC1, MUC16, FcRa, CD44v6, Necint-4, CAIX, CEA, B7-H3, HPV16-E6, HPV16-E7, AFP, NY-ESO-1, MAGEA4, MAGEA3, MAGEA8, PRAME, COL6A3 and WT1.

In some embodiments, the additional domain of the antigen binding domain binds to CD19 or BCMA.

In some embodiments, a bispecific CAR, a bispecific T cell receptor fusion protein or a bispecific TAC comprising a SIRP IgV domain of present disclosure and a second antigen binding domain that binds to a non-CD47 antigen, wherein the SIRP IgV domain is linked through a linker to the N-terminal or C-terminal of the second antigen binding domain.

In some embodiments, a modified cell comprises a CAR, a T cell receptor fusion protein or a TAC comprising a SIRP IgV domain of present disclosure.

In some embodiments, a modified cell comprises a CAR, a T cell receptor fusion protein or a TAC comprising a SIRP IgV domain of present disclosure, wherein the same cell also comprises a second CAR, a second T cell receptor fusion protein or a second TAC targeting against a non-CD47 antigen.

In certain embodiments, a composition comprises a first population of modified T cells comprising a CAR, a T cell receptor or a TAC comprising a SIRP IgV domain of present disclosure, and comprises a second population of modified T cells comprising a CAR against CD19 and a polynucleotide comprising a sequence encoding IL-6 and IFN-γ driven by a NEAT promoter, wherein the second population of modified T cells express and secrete IL-6 and IFN-γ in response to activation of the modified T cells.

In some embodiments, the synthetic receptor is a chimeric antigen receptor (CAR) comprising an amino acid sequence selected from the group consisting of SEQ ID NO: 254 to 267, a T cell receptor (TCR) fusion protein comprising an amino acid sequence selected from the group consisting of SEQ ID NO: 268 to 279, or a T cell antigen coupler (TAC) comprising an amino acid sequence selected from the group consisting of SEQ ID NO: 280 to 285.

In one aspect, the present disclosure provides a nucleic acid comprising a sequence encoding a SIRP IgV monomer, a SIRP IgV multimer polypeptide, a SIRP IgV domain-comprising fusion polypeptide, a fusion protein, the protein of a protein drug conjugate, and/or a synthetic receptor of present disclosure.

In some embodiments, the nucleic acid is selected from the group consisting of a DNA and a RNA.

In one aspect, the present disclosure provides an expression vector comprising a nucleic acid or nucleic acids of the present disclosure, wherein the expression vector is selected from the group consisting of plasmids, lentivirus vectors, gamma retrovirus vectors, foamy virus vectors, adeno associated virus vectors, adenovirus vectors, pox virus vectors, herpes virus vectors, engineered hybrid viruses, and transposon mediated vectors.

In one aspect, the present disclosure provides a modified cell comprising a nucleic acid, and/or a expression vector of the present disclosure.

In one aspect, the present disclosure provides a composition of cells comprising a population of modified cells comprising a CAR, a T cell receptor fusion protein or a TAC comprising a SIRP IgV domain of present disclosure, wherein the same population of cells and/or a different population of cells comprise 1) a nucleic acid and/or an expression vector comprising a nucleic acid sequence encoding a second CAR, a second T cell receptor fusion protein or a second TAC targeting against CD19, CD20, CD22, CD37, BCMA or GPRCD5, and/or 2) a nucleic acid and/or an expression vector comprising a nucleic acid sequence encoding a therapeutic agent.

In some embodiments, a second CAR comprises an amino acid sequence of SEQ ID NO: 286.

In some embodiments, the therapeutic agent is one or more cytokines selected from the group consisting of IL-6, IFN-γ and IL-12.

In some embodiments, the nucleic acid encoding the therapeutic agent comprises a promoter sequence comprising SEQ ID NO: 287, wherein the therapeutic agent is expressed and secreted in response to activation of the modified cell.

In some embodiments, the nucleic acid encoding the therapeutic agent encodes an amino acid sequence of SEQ ID NO: 288 and/or 289.

In some embodiments, the nucleic acid encoding the therapeutic agent comprises a nucleic acid sequence of SEQ ID NO: 290.

In some embodiments, the modified cell comprises a T cell, NK cell, NKT cell, cytokine-induced killer (CIK) cell, mucosal-associated invariant T (MA1T) cell, monocyte, macrophage, dendritic cell, B cell, granulocyte, neutrophil, innate lymphoid cell (ILC), mesenchymal stem cell (MSC) and/or induced pluripotent stem cell (iPSC).

In some embodiments, the modified cell comprises a T cell, NK cell, NKT cell, CIK cell, macrophage, or iPSC.

In some embodiments, the T cell comprises αβ T cell, γδ T cell, double negative T cell and/or Treg cell. In some embodiments, the cell is an autologous or allogeneic cell.

In one aspect, the present disclosure provides a pharmaceutical composition comprises the SIRP IgV monomer, the SIRP IgV multimer polypeptide, the fusion polypeptide, the fusion protein, the protein drug conjugate, the nucleic acid, the vector, the modified cell, and/or the composition of cells, and the pharmaceutically acceptable carrier.

In some embodiments, a method of treating a CD47-expressing disease in a mammal comprising administrating an effective amount of T cells comprising a synthetic antigen-binding receptor comprising a CD47-binding SIRP IgV domain, wherein the CD47-binding T cells with the synthetic antigen-binding receptor avoids or reduces fratricide of CD47-positive T cells at physiological pH, wherein the SIRP IgV domain of the T cells with the synthetic antigen-binding receptor comprises a mutation of K53H, R69H, Q52H and/or K68H.

### Treatment

In one aspect, the present disclosure provides a method of treating a CD47-expressing disease in a mammal comprising administering an effective amount of the SIRP IgV monomer, the SIRP IgV multimer polypeptide, the fusion polypeptide, the fusion protein, the protein drug conjugate, the nucleic acid, the vector, the modified cell, the composition of cells, and/or the pharmaceutical composition, to a subject, e.g. a mammal, in need thereof.

In some embodiments, the CD47-expressing disease is a disease of cancer, fibrosis, atherosclerosis, inflammation or senescence.

In some embodiments, the CD47-expressing disease is a disease of cancer, comprising ovarian cancer, endometrial cancer, uterine cancer, cervical cancer, breast cancer, gastric cancer, esophageal cancer, lung cancer, head and neck cancer, colorectal cancer, liver cancer, bone cancer, sarcoma, osteosarcoma, brain cancer, multiple myeloma, acute myeloid leukemia (AML), myelodysplastic syndrome, adrenal cancer, anal cancer, bile duct cancer, bladder cancer, basal cell skin cancer, chondrosarcoma, Ewing's sarcoma, eye cancer, gallbladder cancer, gastrointestinal carcinoid tumors, gastrointestinal stromal tumor (GIST), glioma, glioblastoma, hepatocellular cancer, kaposi sarcoma, kidney cancer, laryngeal and hypopharyngeal cancer, leiomyosarcoma, non-small cell lung cancer (NSCLC), small cell lung cancer (SCLC), lung carcinoid tumor, mesothelioma, Merkel cell carcinoma, melanoma, nasal cavity and paranasal sinus cancer, nasopharyngeal cancer, neuroendocrine cancer, neuroblastoma, oral cavity and oropharyngeal cancer, pancreatic cancer, penile cancer, pituitary tumors, prostate cancer, renal cancer, retinoblastoma, rhabdomyosarcoma, salivary gland cancer, squamous cell skin cancer, small intestine cancer, stomach cancer, testicular cancer, thymus cancer, thyroid cancer, uterine sarcoma, urinary tract cancer, vaginal cancer, vulvar cancer, Wilms' tumor, diffuse large B-cell lymphoma (DLBCL), follicular lymphoma, hairy-cell leukemia, Hodgkin's lymphoma, leukemia, acute lymphocytic leukemia (ALL), chronic lymphocytic leukemia (CLL), chronic myeloid leukemia (CML), chronic myelomonocytic leukemia (CMML), lymphoma, Mantle cell lymphoma (MCL), marginal zone lymphoma (MZL), myeloma, Non-Hodgkin lymphoma (NHL), plasmacytoma, or Waldenstrom macroglobulinemia

In some embodiments, the CD47-expressing disease is a fibrotic disease of lung, liver, heart, kidney, skin, eye, muscle and/or connective tissues, comprising idiopathic pulmonary fibrosis, liver fibrosis in nonalcoholic fatty liver disease (NAFLD) and non-alcoholic steatohepatitis (NASH), scleroderma and Systemic Sclerosis.

In some embodiments, the mammal is a human.

### Combination Therapy

In one aspect, the present disclosure provides a method of combination therapy in human comprising administering a therapeutically effective amount of a SIRP IgV monomer of present disclosure, a SIRP IgV multimer polypeptide of present disclosure, a fusion polypeptide of present disclosure, a fusion protein of present disclosure, a protein drug conjugate of present disclosure, a nucleic acid of present disclosure, a vector of present disclosure, and/or a cell therapy comprising a synthetic receptor of present disclosure, and a therapeutically effective amount of another therapy. In some embodiments, another therapy comprises administration of one or more of chemotherapy, molecular targeted therapy, biologics therapy, immunotherapy and radiotherapy.

A method of combination therapy in human comprising administering a therapeutically effective amount of a SIRP IgV monomers, a SIRP IgV multimer polypeptide, a fusion polypeptide, a fusion protein, a protein drug conjugate, a nucleic acid, an expression vector, a modified cell, a composition of cells, and/or a pharmaceutical composition of preceding claims, and a therapeutically effective amount of another therapy.

In some embodiments, another therapy comprises administration of one or more of chemotherapy, molecular targeted therapy, biologics therapy, immunotherapy and radiotherapy. In some embodiments, another therapy comprises a chemotherapy, molecular targeted therapy, biologics therapy, immunotherapy and/or radiotherapy that enhance pro-phagocytic signal and/or inhibit anti-phagocytic signal.

In some embodiments, another therapy comprises a chemotherapy, molecular targeted therapy, biologics therapy, immunotherapy and/or radiotherapy that comprise cell adhesion among diseased cells and/or between diseased cells and extracellular matrix.

### INCORPORATION BY REFERENCE

All publications, literature, patents, and patent applications mentioned in this disclosure, including in all tables and figures, are herein incorporated by reference as if each individual publication, patent, or patent application was specifically and individually indicated to be incorporated by reference.

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in other systems and methods. Before explaining the disclosed embodiments of the present invention in detail, it is to be understood that the invention is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1: FIG. 1A is alignment of human SIRPα variant IgV domains, including 4 preceding amino acids from the signal peptide sequence. The residues that show difference among the variants are shadowed and marked as green (the same as SIRPαV1), blue (the same as SIRPαV2) and orange (unique to the specific variant). FIG. 1B is alignment of IgV domains of human SIRPα (representative SIRPαV1 and V2), SIRPβ1 and its representative mutants, SIRPβ2 and its representative mutants, as well as SIRPγ and its representative mutants.

FIG. 2 is an illustration of exemplary structural format variants FV-1 to FV-14.

FIG. 3: is illustration of exemplary structural format variants FV-15 to FV-22. FIG. 3B is illustration of exemplary structural format variants FV-23 to FV-28.

FIG. 4: FIG. 4A is illustration of exemplary structural format variants FV-29 to FV-35. FIG. 4B is illustration of exemplary structural format variants FV-36 to FV-42. FIG. 4C is illustration of exemplary structural format variants FV-43 to FV-47.

FIG. 5: FIG. 5A is illustration of exemplary structural format variants FV-48 to FV-57. FIG. 5B is illustration of exemplary structural format variants FV-58 to FV-63.

FIG. 6: FIG. 6A is illustration of exemplary structural format variants FV-64 to FV-78. FIG. 6B is illustration of exemplary structural format variants FV-79 to FV-92. FIG. 6C is illustration of exemplary structural format variants FV-93 to FV-100.

FIG. 7 is illustration of exemplary structural format variants FV-101 to FV-113.

FIG. 8 is illustration of exemplary structural format variant FV-114.

FIG. 9 is illustration of exemplary structural format variants FV-128 to FV-135.

FIG. 10: FIG. 10A is illustration of exemplary structural format variants FV-136 to FV-142. FIG. 10B is illustration of exemplary structural format variants FV-143 to FV-149.

FIG. 11: FIG. 11A is illustration of exemplary structural format variants FV-150 to FV-155. FIG. 11B is illustration of exemplary structural format variants FV-156 to FV-167.

FIG. 12: FIG. 12A is illustration of exemplary structural format variants FV-168 to FV-171. FIG. 12B is illustration of exemplary structural format variants FV-172 to FV-182. FIG. 12C is illustration of exemplary structural format variants FV-183 to FV-195.

FIG. 13: FIG. 13A shows SDS-PAGE results of SIRPαV2 wild type and variant SIRPαV2 IgV-IgG1 Fe fusion proteins. R: reducing, NR: Non-reducing. FIG. 13B shows SEC-HPLC profiles of wild type and variant SIRPαV2 IgV-IgG1 Fc fusion proteins.

FIG. 14: FIG. 14A shows FACS binding on Raji cells of wild type and variant SIRPαV2 IgV-IgG1 Fc fusion proteins at physiological pH 7.2. FIG. 14B shows FACS binding on Raji cells of wild type and variant SIRPαV2 IgV-IgG1 Fe fusion proteins at an acidic pH 6.0. FIG. 14C-D show FACS binding on Raji cells of wild type and variant SIRPαV2 IgV-IgG1 Fc fusion proteins at both physiological and acidic pH.

FIG. 15: FIG. 15A shows FACS binding on Raji cells of wild type SIRPαV2 IgV-IgG1 Fc fusion protein SIN-300 and variant SIRPαV2 IgV-IgG1 Fc fusion proteinSIN-301 and SIN-304 at an acidic pH and physiological pH. FIG. 15B shows FACS binding on Raji cells of wild type SIRPαV2 IgV-IgG1 Fc fusion protein SIN-300 and variant SIRPαV2 IgV-IgG1 Fe fusion protein SIN-302 and SIN-303 at an acidic pH and physiological pH. FIG. 15C shows FACS binding on SK-OV-3 cells of wild type and variant SIRPαV2 IgV-IgG1 Fc fusion proteins at an acidic pH and physiological pH.

FIG. 16: FIG. 16A shows ELISA of wild type and variant SIRPαV2 IgV-IgG1 Fe fusion proteins with human CD47 protein at physiological pH 7.2. SIRPαV2 SIRPαV2 IgV-IgG1 Fc fusion proteins were immobilized and recombinant human CD47 protein with His-tag was used to detect monovalent binding with the SIRPαV2 IgV-IgG1 Fc fusion proteins SIRPαV2. FIG. 16B shows ELISA of wild type and variant SIRPαV2 IgV-IgG1 Fc fusion proteins with human CD47 protein at an acidic pH 6.0. SIRPαV2 IgV-IgG1 Fe fusion proteins were immobilized and recombinant human CD47 protein with His-tag was used to detect monovalent binding with the SIRPαV2 IgV-IgG1 Fc fusion proteins SIRPαV2. FIG. 16C shows ELISA of wild type and variant SIRPαV2 IgV-IgG1 Fc fusion proteins against immobilized human CD47 protein at an acidic pH 6.0 and physiological pH 7.3. Recombinant human CD47 protein was immobilized and the wild type and variant SIRPαV2 IgV-IgG1 Fc fusion proteins comprising bivalent or monovalent SIRPαV2 IgV domain were used to detect binding with the immobilized CD47.

FIG. 17: FIG. 17A shows FACS binding on primary human platelets of wild type and variant SIRPαV2 IgV-IgG1 Fc fusion proteins at physiological pH 7.3. FIG. 17B shows FACS binding on primary human platelets of wild type and variant SIRPαV2 IgV-IgG1 Fe fusion proteins at physiological pH 7.3 at high concentrations. FIG. 17C shows FACS binding on primary human T cells of wild type and variant SIRPαV2 IgV-IgG1 Fc fusion proteins at physiological pH 7.3 and acidic pH 6.0.

FIG. 18: FIG. 18A shows FACS binding on Raji cells of wild type and variant SIRPαV1 IgV-IgG1 Fc fusion proteins at an acidic pH and physiological pH. FIG. 18B shows FACS binding on Raji cells of wild type and variant SIRPαV8 IgV-IgG1 Fe fusion proteins at an acidic pH and physiological pH. FIGS. 18C and 18E show FACS binding on Raji cells of wild type and variant SIRPγ IgV-IgG1 Fc fusion proteins at an acidic pH and physiological pH. FIG. 18D shows FACS binding on Raji cells of parent and variant SIPRβ2 H101D IgV-IgG1 Fc fusion proteins at an acidic pH and physiological pH.

FIG. 19: FIG. 19A shows ADCP activity of wild type and variant SIRPαV2 IgV-IgG1 Fc fusion proteins at physiological pH 7.3 using ADCP Jurkat reporter assay against Raji cells. FIG. 19B shows ADCP activity of wild type and variant SIRPαV2 IgV-IgG1 Fc fusion proteins at pH 6.5 using ADCP Jurkat reporter assay against Raji cells.

FIG. 20: FIG. 20A shows ADCC activity of wild type and variant SIRPαV2 IgV-IgG1 Fe fusion proteins at physiological pH 7.2 using ADCC Jurkat reporter assay against Raji cells. FIG. 20B shows ADCC activity of wild type and variant SIRPαV2 IgV-IgG1 Fe fusion proteins at an acidic pH 6.0 using ADCC Jurkat reporter assay against Raji cells.

FIG. 21: FIG. 21A shows FACS binding on Raji cells of IgG1 Fe fusion proteins of wild type and variant SIRPαV2 IgV with combo mutations (SIN-332 and SIN-333) at an acidic pH and physiological pH. FIG. 21B shows FACS binding on Raji cells of IgG1 Fe fusion proteins of wild type and variant SIRPαV2 IgV with combo mutations (SIN-335, SIN-336 and SIN-337) at an acidic pH and physiological pH. FIG. 21C shows FACS binding on Raji cells of IgG1 Fe fusion proteins of wild type and variant SIRPαV2 IgV with combo mutations (SIN-330) at an acidic pH and physiological pH.

FIG. 22A-E: shows FACS binding on Raji cells of exemplary additional single-mutation variant SIRPαV2 IgV-IgG1 Fc fusion proteins at an acidic pH and physiological pH.

FIG. 23: FIG. 23A-B show FACS binding on Raji cells of IgG1 Fc fusion proteins of wild type and variant SIRPαV2 IgV with combo mutation of K53H+Q37H at an acidic pH and physiological pH compared to single K53H backbone mutation; FIG. 23B shows FACS binding on Raji cells of IgG1 Fc fusion proteins of wild type and variant SIRPαV2 IgV with combo mutation of R69H+Q37H at an acidic pH and physiological pH compared to single R69H backbone mutation. FIG. 23C-D show FACS binding on Raji cells of IgG1 Fe fusion proteins of wild type and variant SIRPαV2 IgV comprising combo mutation of K53H+I31E, or R69H+I31E, or Q52H+I31E, or K68H+I31E at an acidic pH and physiological pH compared to counterpart SIRPαV2 IgV protein comprising single backbone mutation of K53H, R69H, Q52H, or K68H respectively.

FIG. 24: FIG. 24A-P show SDS-PAGE and SEC-HPLC (280 nm) results of various representative proteins of exemplary structural formats of fusion proteins comprising a SIRP IgV domain.

FIG. 25: FIG. 25A shows tetravalent SIRPαV2 IgV K53H-IgG1 Fe fusion protein SIN-368 (as configured in format FV-3) exhibits increased binding with larger binding difference at an acidic pH 6.0 versus physiological pH 7.3 than bivalent SIRPαV2 IgV K53H-IgG1 Fc fusion protein SIN-301 in FACS binding assay against Raji cells. FIG. 25B shows hexavalent SIRPαV2 IgV R69H-IgG1 Fe fusion protein SIN-370 (as configured in format FV-114) exhibits increased binding but with smaller binding difference at an acidic pH 6.0 versus physiological pH. 7.3 than bivalent SIRPαV2 IgV R69H-IgG1 Fc fusion protein SIN-302 in FACS binding assay against Raji cells.

FIG. 26: FIG. 26A shows ELISA against immobilized human CD47 protein at pH 7.3 of wild type SIRPαV2 IgV fused to the N-terminal of light chain (LC) and/or heavy chain (HC) of anti-PD-L1 antibody. FIG. 26B shows FACS binding on Raji cells at pH 7.3 of wild type SIRPαV2 IgV fused to the N-terminal of LC and/or HC of anti-PD-L1 antibody. FIG. 26C shows ELISA against immobilized human PD-L1 protein at pH 7.3 of wild type SIRPαV2 IgV fused to the N-terminal of LC and/or HC of anti-PD-L1 antibody. FIG. 26D shows FACS binding on MC38-hPD-L1 cells at pH 7.3 of wild type SIRPαV2 IgV fused to the N-terminal of LC and/or HC of anti-PD-L1 antibody. FIG. 26E shows FACS binding on HT-1080 cells at pH 7.3 of wild type SIRPαV2 IgV fused to the N-terminal of LC and/or HC of anti-PD-L1 antibody. FIG. 26F shows ELISA detection of simultaneous binding to human PD-L1 and CD47 protein by MP-5.

FIG. 27: FIG. 27A shows ELISA against immobilized human CD47 protein at pH 6.0 and pH 7.3 of fusion proteins comprising two or four SIRPαV2 IgV domains with K53H or R69H mutation configured in different formats. FIG. 27B shows ELISA against immobilized human CD47 at pH 6.0 and pH 7.3 of fusion proteins comprising four or six SIRPαV2 IgV domains with K53H or R69H mutation configured in different formats. FIG. 27C shows ELISA against immobilized human CD47 at pH 6.0 and pH 7.3 of fusion proteins comprising two or four SIRPαV2 IgV domains with K53H mutation configured in different formats at high concentrations. FIG. 27D shows ELISA against immobilized human CD47 at pH 6.0 and pH 7.3 of fusion proteins comprising four SIRPαV2 IgV domains with K53H or R69H mutation in different formats.

FIG. 28: FIG. 28A shows FACS binding on Raji cells at pH 6.0 and pH 7.3 of fusion proteins comprising two, four or six SIRPαV2 IgV domains with K53H or R69H mutation configured in different formats. FIG. 28B shows FACS binding on Raji cells at pH 6.0 and pH 7.2 of fusion proteins comprising two, four or six SIRPαV2 IgV domains with K53H or R69H mutation configured in different formats. FIG. 28C shows FACS binding on SK-OV-3 cells at pH 6.0 and pH 7.3 of fusion proteins comprising two or four SIRPαV2 IgV domains with K53H or R69H mutation configured in different formats.

FIG. 29: FIG. 29A shows FACS binding at physiological pH 7.3 on primary human platelets of fusion proteins comprising one, two or four SIRPα IgV domains with K53H or R69H mutation configured in different formats. FIG. 29B shows FACS binding at physiological pH 7.3 on primary human platelets of fusion proteins comprising two or four SIRPα IgV domains with K53H or R69H mutation configured in different formats at high concentrations. FIG. 29C shows FACS binding at physiological pH 7.3 on primary human T cells of fusion proteins comprising two or four SIRPα IgV domains with K53H or R69H mutation configured in different formats at high concentrations.

### DETAILED DESCRIPTION

The present disclosure relates to compositions and methods for preferentially targeting CD47 in diseased tissues while reducing/avoiding targeting normal tissues. CD47 has emerged as a highly attractive therapeutic target for cancer, not only as a functional target for promoting phagocytosis of cancer cells by blocking the CD47's interaction with SIRPα, but also as a universal tumor antigen target for directing various therapeutic modalities such as cytotoxic agents and radioligands to cancer cells, given the high and also often uniform expression of CD47 in a wide variety of cancers. However, the ubiquitous expression of CD47 across normal tissues presents a major obstacle for targeting CD47. Multiple anti-CD47 antibody therapeutics stopped clinical development due to safety issues, prominently hemolytic or anemic toxicities due to hemagglutination and/or phagocytic clearance of red blood cells (RBC) induced by the anti-CD47 antibody therapeutics. While a number of anti-CD47 therapeutics for cancer have managed to advance in the clinic through using a priming plus maintenance dosing schedule (e.g. for magrolimab) or using CD47-binders with weak/minimal binding to human RBC (e.g. TTI-621 and TTI-622 using the CD47-binding IgV extracellular domain of SIRPα with minimal native binding to RBC), these therapeutics still bind to CD47 on a broad range of other normal cells and tissues with associated safety risks. For example, thrombocytopenia and neutropenia are commonly reported adverse events for these anti-CD47 therapeutics in clinical development.

Consequently, these current anti-CD47 therapeutics are mostly utilized to functionally block the CD47 “don't eat me” signal for promoting phagocytosis, but not for directing other therapeutic modalities such as cytotoxic agents and radioligands to CD47-expressing disease tissue or cells such as cancer cells, due to on-target toxicity risks to the broad CD47-expressing normal tissues. Furthermore, these CD47-blocking protein therapeutics, usually formatted with a Fc of inert/weak effector function (e.g. hIgG2, hIgG4 or silent hIgG1 Fc), mostly only block the CD47 anti-phagocytic signal, without providing a potent pro-phagocytic signal (e.g. through using hIgG1 Fc with potent ADCP effector function) that's also critical to promote phagocytosis. While TTI-621 uses a wild type hIgG1 Fc (U.S. Pat. No. 9,969,789B2), its dose escalation stopped at a dose of only 2 mg/kg in Phase I clinical trial, versus its counterpart TTI-622 with a weak hIgG4 Fc (U.S. Pat. No. 10,906,954B2) has not reached maximal tolerated dose at 18 mg/kg dose.

Described herein directs to pH-sensitive CD47-binding SIRP IgV domains as well as proteins, protein drug conjugates and synthetic receptor constructs comprising said CD47-binding SIRP IgV domains with differential targeting of CD47 in diseased tissue or cells over normal tissues, and their compositions and methods of use and production related thereto.

### Definition

As used herein, the singular form “a”, “an” and “the” include plural references unless the context clearly dictates otherwise. For example, the term “a substitution” or “at least one substitution” may include a plurality of substitutions, and the term “a domain” or “the domain” may include a plurality of domains.

As used herein, the term “about” or “approximately” refers to a quantity, level, value, number, frequency, percentage, dimension, size, amount, weight, or length that varies by as much as 30, 25, 20, 25, 10, 9, 8, 7, 6, 5, 4, 3, 2, or 1% to a reference quantity, level, value, number, frequency, percentage, dimension, size, amount, weight, or length. In particular embodiments, the terms “about” or “approximately” when preceding a numerical value indicates the value plus or minus a range of 15%, 10%, 5%, or 1%.

As used herein, the terms “comprise”, “comprises”, and “comprising”, mean to imply the inclusion of a stated step or element or group of steps or elements, but not the exclusion of any other step or element or group of steps or elements. For example, “a variant comprises a substitution” may include further substitutions.

Reference throughout this disclosure to “one embodiment,” “an embodiment,” “some embodiment”, “a particular embodiment,” “a related embodiment,” “a certain embodiment,” “an additional embodiment,” or “a further embodiment,” or combinations thereof means that a particular feature, structure, or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “CD47” means CD47 (cluster of differentiation 47), also known as IAP (integrin-associated protein). In human, four isoforms of CD47 protein have been reported, with NCBI Accession number of NP_001768.1 (isoform 1), NP_00942088.1 (isoform 2), NP_001369235.1 (isoform 3) and XP_005247966.1 (isoform X1). The amino acid sequences of the four CD47 isoforms only differ in the length of the C-terminal located inside cytosol when CD47 is natively expressed as a transmembrane protein.

As used herein, the term “SIRP” means “Signal regulatory protein”, including SIRPα, SIRPβ, and SIRPγ, which are a family of transmembrane glycoproteins with three extracellular Ig-like domains, including one IgV domain at the N-terminal followed with two IgC domains. The IgV extracellular domain serves as the direct CD47-binding domain of the SIRP proteins (Barclay et al., Nat Rev Immunol, 2006). As used herein, the term “SIRP IgV” means the IgV extracellular domain of the SIRP proteins, including the IgV extracellular domain of SIRPα, SIRPβ, and SIRPγ.

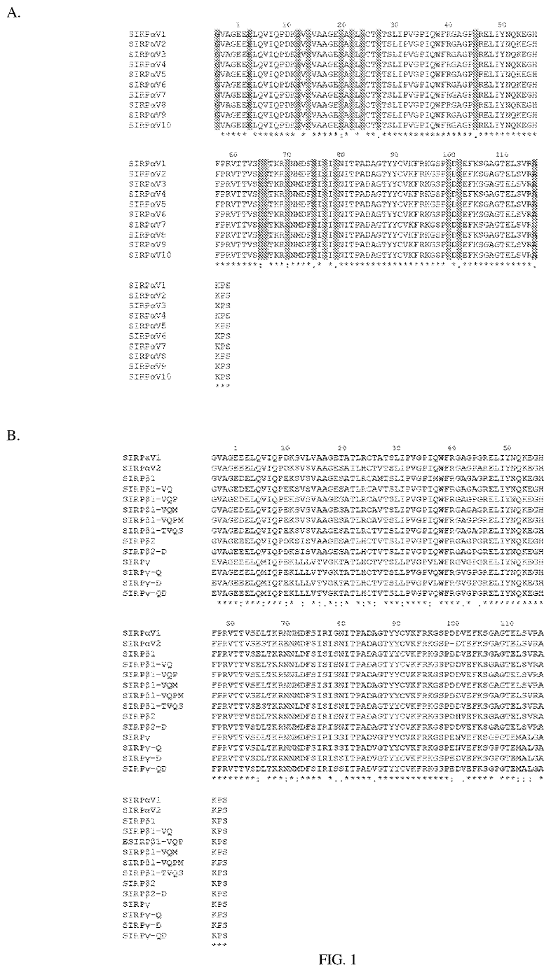

SIRPα comprises 10 members, namely SIRPαV1 to V10 (FIG. 1A), whose IgV domains (SEQ ID NO: 3 to 11, see Table 1 below) all bind to CD47. SIRPαV1, V2 and V8 are reported to be most prevalent SIRPα variants in human (Voets et al., J Immunother Cancer, 2019). The IgV domain sequences of human SIRPαV1 and SIRPαV2 comprise 118 and 119 amino acids respectively, and they differ in 13 amino acids (as shown in FIG. 1A) (Hatherley et al., J Biol Chem, 2014). Notably, the IgV sequences of human SIRPαV5, V6 and V9 variants each only differ in one amino acid from that of human SIRPαV1, and the IgV sequence of human SIRPαV3 and V7 variants each only differ in one amino acid from that of human SIRPαV2, while human SIRPαV10 only differs in one amino acid in the signal peptide sequence from that of human SIRPαV2 (as shown in FIG. 1A) (Hatherley et al., J Biol Chem, 2014). Meanwhile, human SIRPαV4 and SIRPαV8 variants differ from both human SIRPαV1 and SIRPαV2, comprising a mixture of the 13 amino acid differing between human SIRPαV1 and SIRPαV2 (as shown in FIG. 1A) (Hatherley et al., J Biol Chem, 2014).

SIRPβ comprises SIRPβ1 (NP_006056.2, SEQ ID NO:1) and SIRPβ2 (NP_001129316.1, SEQ ID NO:2) (Hatherley et al., Mol Cell, 2008). The native IgV domain of SIRPβ1 shows no detectable CD47 binding, but mutation of 2 amino acids of SIRPβ1 to the amino acids at the corresponding positions of SIRPαV2 (M27V+M37Q as shown in FIG. 1B) results in CD47-binding, with additional mutations that could further increases its CD47 binding, as described in the prior arts (Hatherley et al., Mol Cell, 2008, Lee et al., J Immunol, 2007, Liu et al., J Mol Biol, 2007), hereby incorporated by reference. See Table 1 below for such SIRPβ1 sequences (SEQ ID NO: 1 and 12 to 16). Similarly, mutation of 1 amino acid in the IgV domain of SIRPβ2 to the amino acid at the corresponding position of SIRPαV2 (H101D as shown in FIG. 1B) results in CD47-binding of the variant SIRPβ2 IgV domain (Hatherley et al., Mol Cell, 2008). See Table 1 below for such SIRPβ2 sequences (SEQ ID NO:2 and 17). SIRPγ (NP_00061026.2, SEQ ID NO:18) binds to CD47 about ten times weaker than SIRPα, however mutation of the 2 differing amino acids in SIRPγ IgV domain to the amino acid at corresponding positions of SIRPαV2 (L37Q and/or N101D as shown in FIG. 1B) increases CD47-binding of the SIRPγ IgV domain (Hatherley et al., Mol Cell, 2008) (U.S. Pat. No. 9,845,345B2). See Table 1 for such SIRPγ sequences (SEQ ID NO: 18 to 21)

TABLE 1
The amino acid sequences of SIRP IgV

| SEQ ID NO: | Description | Sequence |
|---|---|---|
| 1 | SIRPß1 IgV | EDELQVIQPEKSVSVAAGESATLRCAMTSLIPVGPIMWFRGAGAGRELIYN |
| | WT* | QKEGHFPRVTTVSELTKRNNLDFSISISNITPADAGTYYCVKFRKGSPDDVE |
| | | FKSGAGTELSVRAKPS |
| 2 | SIRPß2 IgV | EEELQVIQPDKSISVAAGESATLHCTVTSLIPVGPIQWFRGAGPGRELIYNQ |
| | WT | KEGHFPRVTTVSDLTKRNNMDFSIRISNITPADAGTYYCVKFRKGSPDHVE |
| | | FKSGAGTELSVRAKPS |
| 3 | SIRPα V1 IgV | EEELQVIQPDKSVLVAAGETATLRCTATSLIPVGPIQWFRGAGPGRELIYNQ |
| | WT | KEGHFPRVTTVSDLTKRNNMDFSIRIGNITPADAGTYYCVKFRKGSPDDVE |
| | | FKSGAGTELSVRAKPS |
| 4 | SIRPα V2 IgV | EEELQVIQPDKSVSVAAGESAILHCTVTSLIPVGPIQWFRGAGPARELIYNQ |
| | WT | KEGHFPRVTTVSESTKRENMDFSISISNITPADAGTYYCVKFRKGSPDTEFK |
| | | SGAGTELSVRAKPS |
| 5 | SIRPα V3 IgV | EEELQVIQPDKSVSVAAGESAILLCTVTSLIPVGPIQWFRGAGPARELIYNQ |
| | WT | KEGHFPRVTTVSESTKRENMDFSISISNITPADAGTYYCVKFRKGSPDTEFK |
| | | SGAGTELSVRAKPS |
| 6 | SIRPα V4 IgV | EEGLQVIQPDKSVSVAAGESAILHCTATSLIPVGPIQWFRGAGPGRELIYNQ |
| | WT | KEGHFPRVTTVSDLTKRNNMDFSIRIGNITPADAGTYYCVKFRKGSPDDVE |
| | | FKSGAGTELSVRAKPS |
| 7 | SIRPα V5 IgV | EEELQVIQPDKFVLVAAGETATLRCTATSLIPVGPIQWFRGAGPGRELIYNQ |
| | WT | KEGHFPRVTTVSDLTKRNNMDFSIRIGNITPADAGTYYCVKFRKGSPDDVE |
| | | FKSGAGTELSVRAKPS |
| 8 | SIRPα V6 IgV | EEELQVIQPDKSVLVAAGETATLRCTATSLIPVGPIQWFRGAGPGRELIYNQ |
| | WT | KEGHFPRVTTVSDLTKRNNMDFPIRIGNITPADAGTYYCVKFRKGSPDDVE |
| | | FKSGAGTELSVRAKPS |
| 9 | SIRPα V7 IgV | EEELQVIQPDKSVSVAAGESAILHCTVTSLIPVGPIQWFRGAGPARELIYNQ |
| | WT | KEGHFPRVTTVSESTKRENMDFSISISNITPADAGTYYCVKFRKGSPDTEFK |
| | | SGAGTELSVRGKPS |
| 10 | SIRPα V8 IgV | EEELQVIQPDKSVLVAAGETATLRCTATSLIPVGPIQWERGAGPARELIYNQ |
| | WT | KEGHFPRVTTVSESTKRENMDFSISISNITPADAGTYYCVKFRKGSPDTEFK |
| | | SGAGTELSVRAKPS |
| 11 | SIRPα V9 IgV | EEELQVIQPDKSVLVAAGETATLRCTATSLIPVGPIQWFRGAGPGRELIYNQ |
| | WT | KEGHFPRVTTVSDLTKRNNMDFSIRISNITPADAGTYYCVKFRKGSPDDVE |
| | | FKSGAGTELSVRAKPS |
| 12 | SIRPß1 IgV | EDELQVIQPEKSVSVAAGESATLRCAVTSLIPVGPIQWFRGAGAGRELIYN |
| | M27V + M37Q | QKEGHFPRVTTVSELTKRNNLDFSISISNITPADAGTYYCVKFRKGSPDDVE |
| | | FKSGAGTELSVRAKPS |
| 13 | SIRPß1 IgV | EDELQVIQPEKSVSVAAGESATLRCAVTSLIPVGPIQWFRGAGPGRELIYNQ |
| | M27V + M37Q + | KEGHFPRVTTVSELTKRNNLDFSISISNITPADAGTYYCVKFRKGSPDDVEF |
| | A44P | KSGAGTELSVRAKPS |
| 14 | SIRPß1 IgV | EDELQVIQPEKSVSVAAGESATLRCAVTSLIPVGPIQWFRGAGAGRELIYN |
| | M27V + M37Q + | QKEGHFPRVTTVSELTKRNNMDFSISISNITPADAGTYYCVKFRKGSPDDV |
| | L72M | EFKSGAGTELSVRAKPS |
| 15 | SIRPß1 IgV | EDELQVIQPEKSVSVAAGESATLRCAVTSLIPVGPIQWERGAGPGRELIYNQ |
| | M27V + M37Q + | KEGHFPRVTTVSELTKRNNMDFSISISNITPADAGTYYCVKFRKGSPDDVE |
| | A44P + L72M | FKSGAGTELSVRAKPS |
| 16 | SIRPß1 IgV | EDELQVIQPEKSVSVAAGESATLRCTVTSLIPVGPIQWFRGAGAGRELIYN |
| | A26T + M27V + | QKEGHFPRVTTVSESTKRNNLDFSISISNITPADAGTYYCVKFRKGSPDDVE |
| | M37Q + L66S | FKSGAGTELSVRAKPS |
| 17 | SIRPß2 IgV | EEELQVIQPDKSISVAAGESATLHCTVTSLIPVGPIQWFRGAGPGRELIYNQ |
| | H101D | KEGHFPRVTTVSDLTKRNNMDFSIRISNITPADAGTYYCVKFRKGSPDDVE |
| | | FKSGAGTELSVRAKPS |
| 18 | SIRPγ IgV | EEELQMIQPEKLLLVTVGKTATLHCTVTSLLPVGPVLWFRGVGPGRELIYN |
| | | QKEGHFPRVTTVSDLTKRNNMDFSIRISSITPADVGTYYCVKFRKGSPENV |
| | | EFKSGPGTEMALGAKPS |
| 19 | SIRPγ IgV | EEELQMIQPEKLLLVTVGKTATLHCTVTSLLPVGPVQWFRGVGPGRELIYN |
| | L37Q | QKEGHFPRVTTVSDLTKRNNMDFSIRISSITPADVGTYYCVKFRKGSPENV |
| | | EFKSGPGTEMALGAKPS |
| 20 | SIRPγ IgV | EEELQMIQPEKLLLVTVGKTATLHCTVTSLLPVGPVLWFRGVGPGRELIYN |
| | N101D | QKEGHFPRVTTVSDLTKRNNMDFSIRISSITPADVGTYYCVKFRKGSPEDV |
| | | EFKSGPGTEMALGAKPS |
| 21 | SIRPγ IgV | EEELQMIQPEKLLLVTVGKTATLHCTVTSLLPVGPVQWFRGVGPGRELIYN |
| | L37Q + N101D | QKEGHFPRVTTVSDLTKRNNMDFSIRISSITPADVGTYYCVKFRKGSPEDV |
| | | EFKSGPGTEMALGAKPS |

*WT: wild type

As used herein, the term “SIRP IgV monomer” means one single IgV extracellular domain of a SIRP protein, including one single IgV extracellular domain of SIRPα, SIRPβ, or SIRPγ. A wild type SIRP IgV monomer of SIRPα, SIRPβ, or SIRPγ comprises an amino acid sequence selected from the group consisting of SEQ ID NO: 1 to 11 and 18. Optionally, a SIRP IgV monomer may additionally comprise one or both of the two IgC domains (SEQ ID NO: 114 to 117) of the full extracellular domain of a SIRPα, SIRPβ, or SIRPγ protein.

As used herein, the term “SIRP IgV multimer polypeptide” refers to a polypeptide comprising two or more SIRP IgV monomers serially linked together in the polypeptide preferentially with a linker between the two neighboring SIRP IgV monomers.

As used herein, the term “SIRP IgV domain” refers to a structural or a spatial domain that comprises at least one SIRP IgV monomer. A SIRP IgV domain may comprise one or more SIRP IgV monomers, and the SIRP IgV monomers may be linked together in one polypeptide, or separately located in different polypeptides.

As used herein, the term “fusion polypeptide” means a polypeptide comprising at least two fragments that are not naturally linked to each other in the same polypeptide. For a “fusion polypeptide” of present disclosure, it specifically refers to a fusion polypeptide comprising at least one SIRP IgV domain and at least one non-CD47 binding domain, wherein the SIRP IgV domain of the polypeptide comprises at least one SIRP IgV monomer and the non-CD47 binding domain of the polypeptide comprises a fragment that is not from a SIRP family protein and does not bind to CD47. The non-CD47 binding domain preferentially binds to at least one antigen that is not CD47.

As used herein, the term “fusion protein” means a protein comprising at least two fragments that are not naturally fused in the same protein. For a “fusion protein” of present disclosure, it specifically refers to a protein comprising at least one “fusion polypeptide” of present disclosure. A “fusion protein” of present disclosure comprises at least one SIRP IgV domain comprising at least one SIRP IgV monomer. A “fusion protein” of present disclosure may comprise additional polypeptide that doesn't comprise any SIRP IgV monomer.

As used herein, the term “protein drug conjugate” refers to a protein comprising a drug payload moiety that is covalently linked to the protein through chemical reaction or recombinant expression. For a “protein drug conjugate” of present disclosure, it specifically refers to a protein comprising at least one SIRP IgV monomer of present disclosure and a drug payload conjugate.

As used herein, the term “synthetic receptor” refers to a non-natural chimeric protein receptor that can be expressed on the membrane of a host cell and the polynucleotide that encodes such a chimeric protein receptor. Examples of synthetic receptor include chimeric antigen receptor (CAR), T cell receptor fusion protein (TFP) and T cell antigen coupler (TAC). For a synthetic receptor of present disclosure, it refers to a synthetic receptor comprising at least one SIRP IgV monomer.

As used herein, the term “multispecific” refers to specificity against two or more different targets.

As used herein, the term “format variant”, or “FV” in abbreviation, of a protein refers to the structural configuration of the domain components of the protein, as the exemplary format variants (FVs) illustrated in FIGS. 2 to 12. Format variant of FV-15 to 47 includes sub-variants under one FV-number, for example, FV-29 includes FV-29a, FV-29b, FV-29c and FV-29d. In such case, the FV-number indicates all its sub-variants, for example, FV-29 indicates all FV-29 sub-variants including FV-29a, FV-29b, FV-29c and FV-29d.

As used herein, the term “operably linked” is intended to mean that the two polypeptide fragments are joined into one polypeptide such that the amino acid sequences of the two polypeptide fragments remain in-frame separately.

The term “amino acid” as used herein refers to any organic compound that contains an amino group (—NH2) and a carboxyl group (—COOH), preferably either as free groups or alternatively after condensation as part of peptide bonds. The “twenty naturally encoded polypeptide-forming alpha-amino acids” are understood in the art and refer to: alanine (ala or A), arginine (arg or R), asparagine (asn or N), aspartic acid (asp or D), cysteine (cys or C), gluatamic acid (glu or E), glutamine (gin or Q), glycine (gly or G), histidine (his or H), isoleucine (ile or I), leucine (leu or L), lysine (lys or K), methionine (met or M), phenylalanine (phe or F), proline (pro or P), serine (ser or S), threonine (thr or T), tryptophan (tip or W), tyrosine (tyr or Y), and valine (val or V).

The term “peptide” typically refers to short polypeptides. The term “protein” typically refers to longer polypeptides. The left-hand end of a polypeptide sequence is usually described as the amino-terminus (N-terminus); and the right-hand end of a polypeptide sequence is usually described as the carboxyl-terminus (C-terminus).

The term “antibody” as used herein encompasses any immunoglobulin, monoclonal antibody, polyclonal antibody, multispecific antibody, or bispecific (bivalent) antibody that binds to a specific antigen. A native intact antibody comprises two heavy chains and two light chains. Each heavy chain consists of a variable region (“VH”) and a first, second, and third constant region (CH1, CH2 and CH3), while each light chain consists of a variable region (“VL”) and a constant region (CL). Mammalian heavy chains are classified as α, δ, ε, γ, and μ, and mammalian light chains are classified as λ or κ. The variable regions of the light and heavy chains are responsible for antigen binding. The variable regions in both chains generally contain three highly variable loops called the complementarity determining regions (CDRs) (light (L) chain CDRs including LCDR1, LCDR2, and LCDR3, heavy (H) chain CDRs including HCDR1, HCDR2, HCDR3). The three CDRs are interposed between flanking stretches known as framework regions (FRs), which are more highly conserved than the CDRs and form a scaffold to support the hypervariable loops. Each VH and VL comprises four FRs, and the CDRs and FRs are arranged from amino terminus to carboxy terminus in the order: FR1, CDR1, FR2, CDR2, FR3, CDR3, FR4. The constant regions of the heavy and light chains are not involved in antigen binding, but exhibit various effector functions. Antibodies are assigned to classes based on the amino acid sequence of the constant region of their heavy chain. The boundaries of the amino acid sequence of a particular CDR may be defined or identified by the conventions of any of the well-known systems, including IMGT numering, Kabat numbering and Chothia numbering, as described in Dev. Comp. Immunol., 27, 55-77 (IMGT numbering system), Kabat et al. (1991), Sequences of Proteins of Immunological Interest, 5th ed., Public Health Service, National Institutes of Health, Bethesda, MD (Kabat numbering system), Al-Lazikani et al., (1997), JMB 273, 927-948 (Chothia numbering system), Lefranc et al., (2003), or combined system. Throughout this disclosure, wherein CDR sequences for an antibody are referred to, they are preferably defined by the IMGT CDR definition system, unless specified otherwise. However, it will be obvious to those skilled in the art to define the CDR sequences of an antibody from one definition system to another definition system, for example, from an IMGT CDR definition to a Kabat CDR definition, or from an IMGT CDR definition to a Chothia CDR definition. Thus, wherein a CDR sequence for an antibody is provided based on a specific CDR definition, for example in IMGT CDR definition in this disclosure, it is understood that the CDR sequence is provided as one exemplary CDR sequence using the IMGT CDR definition for illustration and the disclosure does also imply and include any alternative CDR sequence defined using a different CDR definition, as it will be obvious for those skilled in the art to determine the alternative CDR sequences of the antibody using the other definition systems, based on the exemplary CDR sequence provided using the exemplary IMGT CDR definition.

In some embodiments, the antibody is an antigen-binding moiety. Antigen-binding moiety as used herein refers to an antibody fragment formed from a portion of an antibody comprising one or more CDRs, or any other antibody fragment that binds to an antigen but does not comprise an intact native antibody structure. Examples of antigen-binding moiety include, without limitation, a variable domain, a variable region, a diabody, a Fab, a Fab′, a F(ab′)₂, an Fv fragment, a disulphide stabilized Fv fragment (dsFv), a (dsFv)₂, a bispecific dsFv (dsFv-dsFv′), a disulphide stabilized diabody (ds diabody), a multispecific antibody, a camelized single domain antibody, a nanobody, a domain antibody, and a bivalent domain antibody. An antigen-binding moiety is capable of binding to the same antigen to which the parent antibody binds. In certain embodiments, an antigen-binding moiety may comprise one or more CDRs from a particular human antibody grafted to a framework region from one or more different human antibodies. For more and detailed formats of antigen-binding moiety are described in Spiess et al, 2015 (Supra), and Brinkman et al., mAbs, 9 (2), pp. 182-212 (2017), which are incorporated herein by reference.

The term “variable domain” or “variable region” with respect to an antibody as used herein refers to an antibody variable region or a fragment thereof comprising one or more CDRs. Although a variable domain or region may comprise an intact variable region (such as VH or VL), it is also possible to comprise less than an intact variable region yet still retain the capability of binding to an antigen or forming an antigen-binding site.

A Fab fragment consists of a monovalent antigen-binding fragment of an antibody molecule, and can be produced by digestion of a whole antibody molecule with the enzyme papain, to yield a fragment consisting of an intact light chain and a portion of a heavy chain. A Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner.

An (Fab′)2 fragment of an antibody can be obtained by treating a whole antibody molecule with the enzyme pepsin, without subsequent reduction. A (Fab′)2 fragment is a dimer of two Fab′ fragments, held together by two disulfide bonds.

An Fv fragment is defined as a genetically engineered fragment containing the variable region of a light chain and the variable region of a heavy chain expressed as two chains. A Fd chain refers to a fragment of antibody heavy chain comprising VH and CH1.

“Fc” with regard to an antibody refers to that portion of the antibody consisting of the second (CH2) and third (CH3) constant regions of a first heavy chain bound to the second and third constant regions of a second heavy chain via disulphide bonding. The Fc portion of the antibody is responsible for various effector functions such as ADCC, ADCP and CDC, but does not function in antigen binding. “CH2 constant region,” which is also referred to as “CH2 domain,” as used herein refers to the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat, E., et al., U.S. Department of Health and Human Services, (1983)). The “CH3 constant region,” which is also referred to as “CH3 domain,” extends from the CH2 domain to the C-terminus of the IgG molecule and comprises approximately 108 amino acids. Certain immunoglobulin classes, e.g., IgM, further include a CH4 region.

“Hinge region” in terms of an antibody includes the portion of a heavy chain molecule that joins the CH1 domain to the CH2 domain. This hinge region is flexible, thus allowing the two N-terminus antigen binding regions to move independently. In some embodiment, the hinge region comprises about amino acids 234 to 243 of Kabat numbering system. In some embodiment, the hinge region comprises about amino acids 226 to 243 of Kabat numbering system. In some embodiment, a Fc comprising the hinge region comprising about amino acids 234 to 243 of Kabat numbering system.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85: 5879 (1988)).

An “antigen” or “Ag” as used herein refers to a compound, composition, peptide, polypeptide, protein, hapten, or substance that can stimulate the production of antibodies or a T cell response in cell culture or in an animal, including compositions (such as one that includes a cancer-specific protein) that are added to a cell culture (such as a bybridoma), or injected or absorbed into an animal. An antigen reacts with the products of specific humoral or cellular immunity (such as an antibody), including those induced by heterologous antigens. The term “fusion” or “fused” when used with respect to amino acid sequences (e.g. peptide, polypeptide, or protein) refers to combination of two or more amino acid sequences, for example by chemical bonding or recombinant means, into a single amino acid sequence that does not exist naturally. A fusion amino acid sequence may be produced by genetic recombination of two encoding polynucleotide sequences, and can be expressed by a method of introducing a construct containing the recombinant polynucleotides into a host cell.

The term “substitution” with regard to amino acid residue as used herein refers to naturally occurring or induced replacement of one or more amino acids with another in a peptide, polypeptide, or protein. Substitution in a polypeptide may result in diminishment, enhancement, or elimination of the polypeptide's function.

The term “humanized” antibody as used herein refers to a chimeric antibody comprising amino acid residues from non-human CDRs and amino acid residues from human FRs. In certain embodiments, a humanized antibody will comprise substantially all of at least one, and typically two, variable domains, in which all or substantially all of the CDRs correspond to those of a non-human antibody, and all or substantially all of the FRs correspond to those of a human antibody. A “humanized form” of an antibody, e.g., a non-human antibody, refers to an antibody that has undergone humanization.

The term “binding” as used herein refers to interaction of a binding domain with an antigen with the interaction depending upon the presence of a particular structure (e.g., an antigenic determinant or epitope) on the antigen. For example, an antibody variable region or Fv recognizes and binds to a specific protein structure rather than to proteins generally. As used herein, the term “specifically binding” or “binding specifically” means that a binding domain binds to or associates with more frequently, more rapidly, with greater duration and/or with greater affinity with a particular antigen than with other proteins. For example, an antibody variable region or Fv specifically binds to its antigen with greater affinity, avidity, more readily, and/or with greater duration than it binds to other antigens. For another example, an antibody variable region or Fv binds to a cell surface protein (antigen) with materially greater affinity than it does to related proteins or other cell surface proteins or to antigens commonly recognized by polyreactive natural antibodies (i.e., by naturally occurring antibodies known to bind a variety of antigens naturally found in humans). However, “specifically binding” does not necessarily require exclusive binding or non-detectable binding of another antigen, this is meant by the term “selective binding”.

The term “affinity” as used herein refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd).

The term “avidity” refers to the strength of the sum total of noncovalent interactions of two or more antigen binding sites and their binding partner (e.g., an antigen).

The term “valency” refers to the number of a specific domain that a molecule consists of. For example, “monovalent” “bivalent”, “trivalent” or “tetravalent” refers to respectively one, two, three or four such specific domains comprised by the molecule in total.

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “effector functions” as used herein refer to those biological activities attributable to the Fc region of an antibody, which vary with the antibody isotype. Examples of antibody effector functions include: C1q binding and complement dependent cytotoxicity (CDC); Fe receptor binding and antibody-dependent cell-mediated cytotoxicity (ADCC); Fc receptor binding and antibody-dependent cell-mediated phagocytosis (ADCP); down regulation of cell surface receptors (e.g. B cell receptor); activation of cells expressing Fc receptor (e.g. B cells, macrophages, dendritic cells), induction of secretion of cytokines and/or chemokines by cells expressing Fc receptor (e.g. B cells, macrophages, dendritic cells).

The term “phagocytosis” refers to a process by which a substance with size (such as a cell, a fragment of cell, a microbe, or a particle) is engulfed and internalized by a cell. The term “phagocyte” refers to a cell that is capable of phagocytosis.

The term “microenvironment” as used herein means any portion or region of a tissue, organ or body that has constant or temporal, physical or chemical differences from other regions of the tissue, organ or regions of the body. For tumors, the term “tumor microenvironment” as used herein refers to the environment in which a tumor exists, which is the non-cellular area within the tumor and the area directly outside the tumorous tissue but does not pertain to the intracellular compartment of the cancer cell itself. The tumor and the tumor microenvironment are closely related and interact constantly. A tumor can change its microenvironment, and the microenvironment can affect how a tumor grows and spreads. Typically, the tumor microenvironment has a low pH in the range of 5.0 to 6.8, or in the range of 5.8 to 6.8, or in the range of 6.2-6.8. The tumor microenvironment has been discussed in {Gillies, 2002 #874}, hereby incorporated by reference here. The term “non-tumor microenvironment” refers to a microenvironment at a site other than a tumor.

The term “cytotoxic agent” as used herein refers to a substance that inhibits or prevents a cellular function and/or causes cell death or destruction. The term “cytostatic agent” as used herein refers to a compound or composition which arrests growth of a cell either in vitro or in vivo. Thus, a cytostatic agent may be one which significantly reduces the percentage of cells in S phase. The term “chemotherapeutic agent” as used herein refers to a chemical compound useful in the treatment of cancer.

As used herein, the term “chimeric antigen receptor” or “CAR” is defined as a cell surface receptor that comprises an extracellular target-binding domain, a transmembrane domain, and an intracellular signaling domain.

The term “vector” as used herein refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” as used herein are used interchangeably and refer to cells into which exogenous nucleic acid has been introduced, including the progeny of such cells.

As used herein, the term “subject” is used interchangeably with “patient” and may be a mammal who is in need of prevention or treatment of cancer, such as primates (for example, humans), companion animals (for example, dogs and cats), livestock (for example, cows, pigs, horses, sheep, and goats), and laboratory animals (for example, rats, mice, and guinea pigs). In an embodiment of the present disclosure, the subject is a human.

As used herein, the term “treatment” generally means obtaining a desired pharmacological and/or physiological effect. The effect may be therapeutic in terms of partially or completely curing a disease and/or adverse effect attributed to the disease. Desirable therapeutic effects include, but are not limited to, prevention of onset or recurrence of disease, alleviation of symptoms, diminishment of any direct or indirect pathological consequences of the disease, prevention of metastasis, decreasing the rate of disease progression, amelioration or slowing of the disease state, and remission or improved prognosis. Preferably, the “treatment” may refer to medical intervention of a disease or disorder that has already developed. As used herein, the term “prevention” relates to a prophylactic treatment, that is, to a measure or procedure, the purpose of which is to prevent, rather than to cure a disease. “Prevention” means that a desired pharmacological and/or physiological effect is obtained which is prophylactic in terms of completely or partially preventing a disease or symptom thereof. As used herein, “preventing or treating cancer” may include inhibiting proliferation, survival, metastasis, recurrence, or therapy resistance of cancer. Such a method may comprise a step of administering the immune cells of the present disclosure to a subject in need of prevention or treatment of cancer. Accordingly, there is provided a use of a composition that comprises the immune cells as an active ingredient, for preventing or treating cancer.

As used herein, the term “administration” means providing a substance to a subject to achieve a prophylactic or therapeutic purpose (for example, prevention or treatment of cancer). As used herein, the term “cancer” refers to a physiological condition that is typically characterized by unregulated cell growth in mammals. The cancer to be prevented or treated in the present disclosure may include, depending on the site of occurrence, colorectal cancer, small intestine cancer, rectal cancer, colon cancer, thyroid cancer, endocrine adenocarcinoma, oral cancer, tongue cancer, pharyngeal cancer, laryngeal cancer, esophageal cancer, cervical cancer, uterine cancer, fallopian tube cancer, ovarian cancer, brain cancer, head and neck cancer, lung cancer, lymph gland cancer, gallbladder cancer, bladder cancer, kidney cancer, liver cancer, pancreatic cancer, prostate cancer, skin cancer (or melanoma), breast cancer, stomach cancer, bone cancer, blood cancer, and the like. However, any cancer can be included therein as long as it expresses an antigen protein on the surface of cancer cells. In an embodiment, the cancer may include at least any one selected from the group consisting of optionally colorectal cancer, rectal cancer, colon cancer, thyroid cancer, oral cancer, pharyngeal cancer, laryngeal cancer, cervical cancer, brain cancer, lung cancer, ovarian cancer, bladder cancer, kidney cancer, liver cancer, pancreatic cancer, prostate cancer, skin cancer, tongue cancer, breast cancer, uterine cancer, stomach cancer, bone cancer, and blood cancer. In another embodiment, the cancer may be a solid cancer.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro AR, I9th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

The term “therapeutically effective amount” of a therapeutic agent or treatment is meant a sufficient amount of the therapeutic agent or treatment to have a therapeutic effect in the subject treated, at a reasonable benefit/risk ratio applicable to any medical treatment. It will be understood, however, that the total daily usage of the therapeutic agent or treatment will be decided by the attending physician within the scope of sound medical judgment. The specific therapeutically effective dose level for any particular patient will depend upon a variety of factors including the disorder being treated and the severity of the disorder; activity of the specific antibody employed; the specific composition employed, the age, body weight, general health, sex and diet of the patient; the time of administration, route of administration, and rate of excretion of the specific antibody employed; the duration of the treatment; drugs used in combination or coincidental with the specific antibody employed; and like factors well known in the medical arts. For example, it is well known within the skill of the art to start doses of the compound at levels lower than those required to achieve the desired therapeutic effect and to gradually increase the dosage until the desired effect is achieved.

In one aspect, the present disclosure provides a variant of CD47-binding SIRP IgV, wherein the variant shows higher binding affinity to CD47 at an acidic pH than at physiological pH, wherein the acidic pH is less than 7.0, preferable 5.5 to 7.0, and the physiological pH is 7.2 to 7.5. In some embodiments, the acidic pH is between about 6.0 to less than 7.0, for example, is about 6.0, 6.1, 6.2, 6.3, 6.4, 6.5, 6.6, 6.7, 6.8, or 6.9. The physiological pH is between about 7.2 to 7.5, for example, is about 7.2, 7.3, 7.4, or 7.5. In some embodiments, the CD47-binding SIRP IgV variant binds to CD47 with a binding affinity that is at least 1.5-fold, at least 2-fold, at least 3-fold, at least 4-fold, at least 5-fold, at least 6-fold, at least 7-fold, at least 8-fold, at least 9-fold, or at least 10-fold higher at an acidic pH than at physiological pH.

### SIRP IgV Monomer

In some embodiments, a IgV extracellular domain of the Signal-regulatory protein (SIRP), hereafter referred to as SIRP IgV, comprises a IgV extracellular domain derived from a Signal-regulatory protein (SIRP) family protein selected from the group consisting of SIRPα, SIRPβ and SIRPγ.

In some embodiments, the SIRP IgV is selected from the group consisting of SIRPαV1 (SEQ ID NO: 3), SIRPαV2 (SEQ ID NO: 4), SIRPαV3 (SEQ ID NO: 5), SIRPαV4 (SEQ ID NO: 6), SIRPαV5 (SEQ ID NO: 7), SIRPαV6 (SEQ ID NO: 8), SIRPαV7 (SEQ ID NO: 9), SIRPαV8 (SEQ ID NO: 10), SIRPαV9 (SEQ ID NO: 11), SIRPαV10 (SEQ ID NO: 11), SIRPβ1 (SEQ ID NO: 1), SIRPβ1-VQ (SEQ ID NO: 12), SIRPβ1-VQP (SEQ ID NO: 13), SIRPβ1-VQM (SEQ ID NO: 14), SIRPβ1-VQPM (SEQ ID NO: 15), SIRPβ1-TVQS (SEQ ID NO: 16), SIRPβ2 (SEQ ID NO: 2), SIRPβ2-D (SEQ ID NO: 17), SIRPγ (SEQ ID NO: 18), SIRPγ-Q (SEQ ID NO: 19), SIRPγ-D (SEQ ID NO: 20) and SIRPγ-QD (SEQ ID NO: 21).

In some embodiments, the present disclosure provides a variant of CD47-binding IgV extracellular domain of SIRPα or SIRPβ, wherein the variant comprises mutation at one or more amino acid residues selected from the group consisting of I31, Q52, K53, K68 and R69 (residue position numbering based on the residue position in SEQ ID NO: 1 to 17), and wherein the variant shows higher binding affinity to CD47 at an acidic pH than at physiological pH.

In some embodiments, the present disclosure provides a variant of CD47-binding IgV extracellular domain of SIRPγ, wherein the variant comprises mutation at one or more amino acid residues selected from the group consisting of L31, Q52, K53, K68 and R69 (residue position numbering based on the residue position in SEQ ID NO: 18 to 21), and wherein the variant shows higher binding affinity to CD47 at an acidic pH than at physiological pH.

In some embodiments, the present disclosure provides a variant of CD47-binding SIRP IgV, wherein the variant comprises one or more substitutions selected from the group consisting of: K53H, R69H, K68H, Q52H, I31E/L31E and I31D/L31D and wherein the variant shows higher binding affinity to CD47 at an acidic pH than at physiological pH. In some embodiments, a variant of CD47-binding SIRP IgV comprises a substitution of Q52H, K53H, K68H, R69H, I31E/L31E or I31D/L31D. In certain embodiments, a variant of CD47-binding SIRP IgV comprises a substitution selected from the group consisting of Q52H+K68H, K53H+K68H, R69H+K68H, K53H+Q52H, R69H+Q52H, K53H+I31E/L31E, R69H+I31E/L31E, K68H+I31E/L31E, Q52H+I31E/L31E, Q52H+K68H+I31E/L31E, K53H+I31D/L31D, R69H+I31D/L31D, K68H+I31D/L31D, Q52H+I31D/L31D, and Q52H+K68H+I31D/L31D, wherein the “+” indicates concurrent combination mutations in a single SIRP IgV monomer domain. In certain embodiments, a variant of CD47-binding SIRP IgV comprises a substitution selected from the group consisting of K53H+K68H+Q52H, and R69H+K68H+Q52H.

In some embodiments, the present disclosure provides a variant of CD47-binding SIRP IgV, wherein the variant comprises a substitution of I31E, I31D, L31E or L31D, and wherein the variant shows higher binding affinity to CD47 at an acidic pH than at physiological pH.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises mutation at one or more amino acid residues selected from the group consisting of E3/G3, L4, V6/M6, D10/E10, K11, V15, E19/K19, A21, V27/A27, S29, L30, 131/L31, P32, V33, G34, P35, 136/V36, Q37/L37, W38, F39, R40, R46, E47, L48, 149, Y50, N51, Q52, K53, E54, G55, H56, F57, P58, R59, V60, T61, T62, V63, S64, E65/D65, S66/L66, T67, K68, E70/N70, N71, M72/L72, F74, S75/P75, 176, S77/R77, S79/G79, N80/S80, D85, Y89, V92, K93, F94, K96, G97, S98, P99, E102/E103, F103/F104, K104/K105, L111/L112/M112, V113/V114/L114, R114/R115/G115, and K116/K117, wherein the “/” indicates different alternative mutation change at the corresponding amino acid residue position across SIRPα, SIRPβ and SIRPγ IgV domains, due to different parent amino acid residues and/or position numbering for the mutation at the corresponding position, according to the SIRPα, SIRPβ and SIRPγ IgV amino acid sequences provided in SEQ ID NO: 1 to 21. In some embodiments, a variant of CD47-binding SIRP IgV comprises a substitution of I31E, L31E, I31D or L31D, wherein the variant further comprises mutation at one or more amino acid residues selected from the group consisting of E3/G3, L4, V6/M6, D10/E10, K11, V15, E19/K19, A21, V27/A27, S29, L30, P32, V33, G34, P35, 136/V36, Q37/L37, W38, F39, R40, R46, E47, L48, 149, Y50, N51, Q52, K53, E54, G55, H56, F57, P58, R59, V60, T61, T62, V63, S64, E65/D65, S66/L66, T67, K68, R69, E70/N70, N71, M72/L72, F74, S75/P75, 176, S77/R77, S79/G79, N80/S80, D85, Y89, V92, K93, F94, K96, G97, S98, P99, E102/E103, F103/F104, K104/K105, L111/L112/M112, V113/V114/L114, R114/R115/G115, and K116/K117.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, E54D, E54Q, E54N, E54P, E54S, E54G, G55H, G55E, G55D, G55R, G55K, H56P, H56Y, H56R, H56D, H56E, F57H, F57E, F57D, P58H, P58E, P58D, P58R, P58K, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D, wherein the “/” indicates different alternative mutation change at the corresponding amino acid residue position across SIRPα, SIRPβ and SIRPγ IgV domains, due to different parent amino acid residues and/or position numbering for the mutation at the corresponding position, according to the SIRPα, SIRPβ and SIRPγ IgV amino acid sequences provided in SEQ ID NO: 1 to 21.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises an additional mutation of I31E or L31E, wherein the additional mutation of I31E/L31E further lowes the binding of the SIRP IgV variant to CD47 at physiological pH. In some embodiments, a variant of CD47-binding SIRP IgV comprises a mutation of K53H+I31E/L31E. In some embodiments, a variant of CD47-binding SIRP IgV comprises a mutation of R69H+I31E/L31E. In some embodiments, a variant of CD47-binding SIRP IgV comprises a mutation of K68H+I31E/L31E. In some embodiments, a variant of CD47-binding SIRP IgV comprises a mutation of Q52H+I31E/L31E. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises an additional mutation of I31D or L31D, wherein the additional mutation of I31D/L31D further lowes the binding of the SIRP IgV variant to CD47 at physiological pH. In some embodiments, a variant of CD47-binding SIRP IgV comprises a mutation of K53H+I31D/L31D. In some embodiments, a variant of CD47-binding SIRP IgV comprises a mutation of R69H+I31D/L31D. In some embodiments, a variant of CD47-binding SIRP IgV comprises a mutation of K68H+I31D/L31D. In some embodiments, a variant of CD47-binding SIRP IgV comprises a mutation of Q52H+I31D/L31D.

In some embodiments, a variant of CD47-binding SIRP IgV comprises a substitution of I31E/L31E or I31D/L31D, wherein the variant further comprises one or more substitutions selected from the group consisting of E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52H, Q52E, Q52D, Q52G, K53H, K53R, K53E, K53D, ES4H, ES4D, E54Q, E54N, ES4P, ES45, ES4G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68H, K68R, K68A, K68E, K68D, K68I, K68T, R69H, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, I76H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D, wherein the “/” indicates different alternative mutation change at the corresponding amino acid residue position across SIRPα, SIRPβ and SIRPγ IgV domains, due to different parent amino acid residues and/or position numbering for the mutation at the corresponding position, according to the SIRPα, SIRPβ and SIRPγ IgV amino acid sequences provided in SEQ ID NO: 1 to 21, and wherein the variant shows higher binding affinity to CD47 at an acidic pH than at physiological pH.

In some embodiments, a variant of CD47-binding SIRP IgV comprises a substitution selected from the group consisting of K53H, R69H, Q52H, K68H, I31E/L31E, I31D/L31D, Q52H+K68H, K53H+K68H, R69H+K68H, K53H+Q52H, R69H+Q52H, K53H+K68H+Q52H, R69H+K68H+Q52H, K53H+I31E/L31E, R69H+I31E/L31E, K68H+I31E/L31E, Q52H+I31E/L31E, K53H+I31D/L31D, R69H+I31D/L31D, K68H+I31D/L31D, Q52H+I31D/L31D, wherein the variant exhibits higher binding affinity to CD47 at an acidic pH than at physiological pH. In some embodiments, a CD47-binding SIRP IgV variant comprising a substitution selected from the group consisting of K53H, R69H, Q52H, K68H, I31E/L31E, I31D/L31D, Q52H+K68H, K53H+K68H, R69H+K68H, K53H+Q52H, R69H+Q52H, K53H+K68H+Q52H, R69H+K68H+Q52H, K53H+I31E/L31E, R69H+I31E/L31E, K68H+I31E/L31E, Q52H+I31E/L31E, K53H+I31D/L31D, R69H+I31D/L31D, K68H+I31D/L31D, Q52H+I31D/L31D, wherein the CD47-binding SIRP IgV variant monomer exhibits higher binding affinity to CD47 at an acidic pH than at physiological pH and exhibits negligible or low binding to human platelets at physiological pH as measured through a FACS binding assay of present disclosure. In some embodiments, a CD47-binding SIRP IgV variant comprising a substitution selected from the group consisting of K53H, R69H, Q52H, K68H, I31E/L31E, I31D/L31D, Q52H+K68H, K53H+K68H, R69H+K68H, K53H+Q52H, R69H+Q52H, K53H+K68H+Q52H, R69H+K68H+Q52H, K53H+I31E/L31E, R69H+I31E/L31E, K68H+I31E/L31E, Q52H+I31E/L31E, K53H+I31D/L31D, R69H+I31D/L31D, K68H+I31D/L31D, Q52H+I31D/L31D, wherein the CD47-binding SIRP IgV variant monomer exhibits higher binding affinity to CD47 at an acidic pH than at physiological pH and exhibits negligible or low binding to human T cells at physiological pH as measured through a FACS binding assay of present disclosure.

In some embodiments, a variant of CD47-binding SIRP IgV comprises a substitution of K53H, wherein the variant further comprises one substitution selected from the group consisting of Q52H, K68H, E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, ES4H, E54D, E54Q, ES4N, E54P, ES45, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67B, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D.

In some embodiments, a variant of CD47-binding SIRP IgV comprises a substitution of R69H, wherein the variant further comprises one substitution selected from the group consisting of Q52H, K68H, E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, E54D, E54Q, E54N, E54P, ES45, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L660, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D.

In some embodiments, a variant of CD47-binding SIRP IgV comprises a substitution of Q52H, wherein the variant further comprises one substitution selected from the group consisting of K53H, K68H, R69H, E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, E54D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D.

In some embodiments, a variant of CD47-binding SIRP IgV comprises a substitution of K68H, wherein the variant further comprises one substitution selected from the group consisting of K53H, Q52H, R69H, E3H/G3H, E3D/G3D, L4H, L4E, L4D, L4V, L4I, V6H/M6H, V6L/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, E54D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/079H, S79B/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D.

In some embodiments, a variant of CD47-binding SIRP IgV comprises a substitution of Q52H+K68H, wherein the variant further comprises one substitution selected from the group consisting of K53H, Q52H, R69H, E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, E54D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D.

In some embodiments, a variant of CD47-binding SIRP IgV comprises one or more substitution selected from the group consisting of E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E. I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52H, Q52E, Q52D, Q52G, K53H, K53R, K53E, K53D, E54H, E54D, E54Q, ES4N, E54P, E54S, E54G, G55H, G55E, G55D, G55Q, G55N, G55R, G55K, G55Y, H56P, H56Y, H56R, H56D, H56E, F57H, F57E, F57D, P58H, P58E, P58D, P58R, P58K, P58Q, P58N, P58Y, R59H, R59E, R59D, V60H, V60E, V60D, T61H, T61E, T61D, T62H, T

62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68H, K68R, K68A, K68E, K68D, K68I, K68T, R69H, R69E, R69D, E70H/N70H, E70D/N70D, N71H, N71D, N71E, M72H/L72H, M72N/L72N, M72R/L72R, M72E, M72D, M72I/L72I, M72W/L72W, D73E, D73H, D73N, D73Q, D73R, D73K, F74H, F74E, F74D, S75H/P75H, S75E/P75E, SP7D/P75D, I76H, I76E, I76D, S77H/R77H, S77K/R77K, S77E/R77E, S77D/R77D, S77N/R77N, 178H, I78E, I78D, I78R, I78K, I78Q, I78N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, I31H/L31H, I31Y/L31Y, I31W/L31W, V33H, P35H, Q37H/L37H, R40H, R46H, N51H, R59H, S66H/L66H, E70H/N70H, M72H/L72H, K96H, K96R, G97H, S98H, P99H, and K104H/K105H.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of I31E/L31E. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of I31D/L31D. In some embodiments, a variant of CD47-binding SIRP IgV comprises a substitution selected from the group consisting of K53H, R69H, Q52H, K68H, R69H+K68H, K53H+Q52H, R69H+Q52H, K53H+K68H+Q52H, and Q52H+K68H, K53H+K68H, R69H+K68H+Q52H, wherein the variant further comprises one additional substitution of I31H/L31H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of I31Y/L31Y. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of I31W/L31W. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of Q37H/L37H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of R40H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of R46H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of N51H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of R59H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of S66H/L66H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of E70H/N70H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of N80A. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of N80G. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of N80S. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of N80Q. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of K96R. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of G97H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of S98H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of P99H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one additional substitution of K104H/K105H.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, R40H, N51H, S66H/L66H, K96R, G97H, S98H, P99H, and K104H/K105H.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, and R40H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, and N51H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, and P99H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, and K104H/K105H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, and K96R.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, Q37H/L37H, R46H, E70H/N70H, and M72H/L72H.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, and Q37H/L37H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, and R46H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, and E70H/N70H.

In some embodiments, a variant of CD47-binding SIRP IgV comprises one or more substitutions selected from the group consisting of I31E/L31E, I31D/L31D, V33H, P35H, Q37H/L37H, R40H, R46H, N51H, S66H/L66H, E70H/N70H, M72H/L72H, N80A, N80S, N80Q, K96R, K96H, G97H, S98H, P99H, and K104H/K105H.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, I31H/L31H, I31Y/L31Y, I31W/L31W, R59H, N80A, N80S an N80Q,

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31E/L31E, I31D/L31D, and R59H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises substitutions of I31E/L31E and R59H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises substitutions of I31D/L31D and R59H. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises a mutation of R59H. In some embodiments, a variant of CD47-binding SIRP IgV comprises a substitution of R59H. In some embodiments, a variant of CD47-binding IgV extracellular domain of SIRPα or SIRPβ comprises a substitution of R59H and an additional mutation of N80A, N80S or N80Q.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of I31H/L31H, I31Y/L31Y, I31W/L31W, R59H, N80A, N80S, N80G an N80Q, In some embodiments, a variant of a CD47-binding SIRP IgV extracellular domain comprises a substitution of I31W/L31W, wherein the substitution increases binding of the variant to CD47. In some embodiments, a variant of a CD47-binding SIRP IgV extracellular domain comprises a substitution of I31Y/L31Y, wherein the substitution increases binding of the variant to CD47. In some embodiments, a variant of a CD47-binding SIRP IgV extracellular domain comprises a substitution of I31H/L31H. In some embodiments, a variant of a CD47-binding SIRP IgV extracellular domain comprises a substitution of R59H, wherein the substitution increases binding of the variant to CD47. In some embodiments, a variant of CD47-binding IgV extracellular domain of SIRPα or SIRPβ comprising a substitution of I31W/L31W, comprises an additional mutation of N80A, N80S, N80G or N80Q, wherein the additional mutation further increases binding to CD47. In some embodiments, a variant of CD47-binding IgV extracellular domain of SIRPα or SIRPβ comprising a substitution of I31Y/L31Y, comprises an additional mutation of N80A, N80S, N80G or N80Q, wherein the additional mutation further increases binding to CD47. In some embodiments, a variant of CD47-binding IgV extracellular domain of SIRPα or SIRPβ comprising a substitution of I31H/L31H, comprises an additional mutation of N80A, N80S, N80G or N80Q, wherein the additional mutation further increases binding to CD47. In some embodiments, a variant of CD47-binding IgV extracellular domain of SIRPα or SIRPβ comprising a substitution of R59H, comprises an additional mutation of N80A, N80S, N80G or N80Q, wherein the additional mutation further increases binding to CD47. In some embodiments, a variant of a CD47-binding SIRP IgV extracellular domain comprising a substitution of I31W/L31W, I31Y/L31Y or I31H/L31H, comprises an additional mutation of R59H. In some embodiments, a variant of CD47-binding IgV extracellular domain of SIRPα or SIRPβ comprises a substitution of I31W+R59H, and an additional substitution of N80A, N80S, N80G or N80Q. In some embodiments, a variant of CD47-binding IgV extracellular domain of SIRPα or SIRPβ comprises a substitution of I31 Y+R59H, and an additional substitution of N80A, N80S, N80G or N80Q. In some embodiments, a variant of CD47-binding IgV extracellular domain of SIRPα or SIRPβ comprises a substitution of I31H+R59H, and an additional substitution of N80A, N80S, N800 or N80Q. In some embodiments, a variant of CD47-binding IgV extracellular domain of SIRPγ comprises a substitution of L31H, L31Y or L31W. In some embodiments, a variant of CD47-binding IgV extracellular domain of SIRPγ comprises a substitution of L31H, L31Y or L31W, and an additional substitution of R59H.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of L4V, L4I, V6I/M6I, V6L/M6L, A21V, V27I/A27I, V27L/A27L, V27Q/A27Q, I31E/L31E, I31D/L31D, I31R/L31R, I31K/L31K, I31F/L31F, I31T/L31T, I31S/L31S, I31L, V33I, P35G, P35N, Q37A/L37A, Q37V/L37V, Q37W/L37W, E47V, E47L, E47Q, E47Y, Q52G, Q52E, K53R, E54Q, E54S, E54D, E54N, E54G, E54P, H56P, H56R, H56Y, V63I, V63A, E65H/D65H, E65R/D65R, S66T/L66T, S66G/L66G, S66Q/L66Q, S66A/L66A, S66E/L66E, S66W/L66W, T67E, T67W, K68R, K68A, K68E, K68I, K68T, E70D/N70D, M72I/L72I, M72N/L72N, M72W/L72W, M72R/L72R, S77N/R77N, S77K/R77K, S79H/G79H, N80A, N80S, N80Q, V92I, V92S, V92N, F94L, F94V, F103V/F104V, and F103I/F104I, wherein the “/” indicates different alternative mutation change at the corresponding residue position across SIRPα, SIRPβ and SIRPγ IgV domains, due to different parent amino acid residues and/or position numbering for the mutation at the corresponding position, according to the SIRPα, SIRPβ and SIRPγ IgV amino acid sequences provided in SEQ ID NO: 1 to 21.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of V27Q/A27Q, P35G, P35N, Q37A/L37A, Q37V/L37V, E47Y, Q52B, E54P, H56Y, S66E/L66E, S66W/L66W, T67E, T67W, K68A, K68E, K68I, K68T, M72I/L72I, M72N/L72N, M72W/L72W, M72R/L72R, and V92N.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises one or more substitutions selected from the group consisting of L4V, L4I, V6I/M6I, V6L/M6L, A21V, V27I/A27I, V27L/A27L, I31E/L31E, I31D/L31D, I31R/L31R, I31K/L31K, I31F/L31F, I31 T/L31T, I31S/L31S, I31L, V33I, Q37W/L37W, E47V, E47L, E47Q, Q52G, K53R, E54Q, E54S, E54D, E54N, E54G, H56P, H56R, V63I, V63A, E65H/D65H, E65R/D65R, S66T/L66T, S66G/L660, S66Q/L66Q, S66A/L66A, K68R, E70D/N70D, S77N/R77N, S77K/R77K, S79H/G79H, N80A, N80S, N80Q, V92I, V92S, F94L, F94V, F103V/F104V, and F103I/F104I.

In some embodiments, a variant of CD47-binding SIRP IgV comprises a substitution of K53H, K53H+Q52H, K53H+K68H, K53H+R69H, or K53H+K68H+Q52H, wherein the variant further comprises a substitution of V6I/M6I+V27I/A27I+I31F/L31F+E47V+E54S+H56P+S66T/L66T+V92I, or I31F/L31F+E54S+H56P+S66T/L66T, of I31L+E47Q+ES4D+S77N/R77N+V92I, or I31L+V33I+E47V+E54N+V63I+S77K/R77K. In some embodiments, a variant of CD47-binding SIRP IgV comprises substitution of R69H, Q52H, K68H, Q52H+K68H, R69H+K68H, R69H+Q52H, or R69H+K68H+Q52H wherein variant the substitution of further comprises a V6I/M6I+V27I/A27I+31F/L31F+E47V+K53R+E54S+H56P+S66T/L66T+V92I, or I31F/L31F+E54S+H56P+S66T/L66T, or I31L+E47Q+K53R+E54D+S77N/R77N+V92I, or I31L+V33I+E47V+K53R+ES4N+V63I+S77K/R77K.

In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises a substitution of N80A, N80S or N80Q. In some embodiments, a variant of CD47-binding SIRP IgV comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the variant further comprises a mutation of N80A, N80S or N80Q, and one or more additional mutations selected from the group consisting of E3H/G3H, E3D/G3D, L4H, L4E, L4D, L4V, L4I, V6H/M6H, V6L/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, E54D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D.

In some embodiments, a variant of CD47-binding SIRP IgV comprises an amino acid sequence at least 80%, 85%, 90%, 95%, 96%, 97%, 98% or 99% identical to SEQ ID NO: 3 to 81, wherein the variant shows higher binding to CD47 at an acidic pH than at physiological pH.

### SIRP IgV Multimer Polypeptide

In one aspect, the present disclosure provides a SIRP IgV multimer polypeptide comprising two, three, four or more CD47-binding SIRP IgV monomers that are linked together serially from the N-terminal to the C-terminal of the polypeptide. In some embodiments, the CD47-binding SIRP IgV monomer comprises an amino acid sequence selected from the group consisting of SEQ ID NO: 3 to 81. In some embodiments, the two or more CD47-binding SIRP IgV monomers are serially linked through a linker. In certain embodiments, the linker comprises (GGGGS)n, wherein n=1, 2, 3, 4, 5, or 6. In certain embodiments, the linker is GGGGSGGGGSGGGGSGGGGS (SEQ ID NO: 121). In some embodiments, the SIRP IgV multimer polypeptide comprises two, three, four or more CD47-binding SIRP IgV variant monomers, wherein the SIRP IgV variant monomers comprise the same or different mutations selected from the group consisting of K53H, R69H, Q52H, K68H, I31E/L31E, I31D/L31D, Q52H+K68H, K53H+K68H, R69H+K68H, K53H+Q52H, R69H+Q52H, K53H+I31E/L31E, R69H+I31E/L31E, K68H+I31E/L31E, Q52H+I31E/L31E, Q52H+K68H+I31E/L31E, K53H+I31D/L31D, R69H+I31D/L31D, K68H+I31D/L31D, Q52H+I31D/L31D, and Q52H+K68H+I31D/L31D, and wherein the SIRP IgV multimer polypeptide exhibits higher binding to CD47 at an acidic pH than at physiological pH.

In some embodiments, the SIRP IgV multimer polypeptide comprises two CD47-binding SIRP IgV variant monomers, wherein the two SIRP IgV variant monomers comprise the same mutation. In certain embodiments, the SIRP IgV multimer polypeptide comprises two CD47-binding SIRP IgV variant monomers comprising the same mutation selected from the group consisting of K53H, R69H, Q52H, K68H and Q52H+K68H. In certain embodiments, the SIRP IgV multimer polypeptide comprises two CD47-binding SIRP IgV variant monomers comprising the same mutation, wherein the mutation of the N-terminal monomer and the C-terminal monomer of the SIRP IgV multimer polypeptide comprises a substitution of K53H-K53H, R69H-R69H, Q52H-Q52H, K68H-K68H, or Q52H+K68H-Q52H+K68H, wherein the “+” indicates concurrent mutation in a single SIRP IgV monomer, while the “−” indicates separate mutation of two neighboring SIRP IgV monomers serially linked from the N-terminal to the C-terminal, wherein the mutation from the left to the right of the “−” indicates the mutation from the N-terminal monomer to the C-terminal monomer. In certain embodiments, the SIRP IgV multimer polypeptide comprises two CD47-binding SIRP IgV variant monomers comprising the same mutation of K53H, R69H, Q52H, K68H or Q52H+K68H, wherein the two monomers comprise one or more the same additional mutation. In further embodiments, the additional mutation comprises I31E/L31E or I31D/L31D. In some embodiments, the SIRP IgV multimer polypeptide comprises two CD47-binding SIRP IgV variant monomers, wherein the two CD47-binding SIRP IgV variant monomers comprise different mutation. In certain embodiments, the SIRP IgV multimer polypeptide comprises two CD47-binding SIRP IgV variant monomers comprising different mutation, wherein the mutation of the N-terminal monomer and the C-terminal monomer of the SIRP IgV multimer polypeptide comprises a substitution of K53H-R69H (i.e. the N-terminal monomer comprising K53H mutation and the C-terminal monomer comprising R69H mutation), R69H-K53H, Q52H-R69H, R69H-Q52H, K68H-R69H, R69H-K68H, Q52H-K53H, K53H-Q52H, K68H-K53H, K53H-K68H, Q52H-K68H, K68H-Q52H, Q52H+K68H-R69H, R69H-Q52H+K68H, Q52H+K68H-K53H, or K53H-Q52H+K68H. In certain embodiments, the SIRP IgV multimer polypeptide comprises two CD47-binding SIRP IgV variant monomers comprising different mutation, wherein the mutation of the N-terminal monomer and the C-terminal monomer comprises a substitution of K53H-R69H, R69H-K53H, Q52H-R69H, R69H-Q52H, K68H-R69H, R69H-K68H, Q52H-K53H, K53H-Q52H, K68H-K53H, K53H-K68H, Q52H-K68H, K68H-Q52H, Q52H+K68H-R69H, R69H-Q52H+K68H, Q52H+K68H-K53H, or K53H-Q52H+K68H, and the N-terminal monomer and/or the C-terminal monomer comprise one or more additional mutation and wherein the one or more additional mutation of the N-terminal monomer and the C-terminal monomer are the same or different. In further embodiments, the additional mutation comprises I31E/L31E or I31D/L31D. In certain embodiments, the SIRP IgV multimer polypeptide comprises two CD47-binding SIRP IgV variant monomers, wherein only one of the two SIRP IgV monomers comprises a mutation of K53H, R69H, Q52H, K68H, Q52H+K68H, K53H+K68H, R69H+K68H, K53H+Q52H, R69H+Q52H, K53H+K68H+Q52H, and R69H+K68H+Q52H. In certain embodiments, the SIRP IgV multimer polypeptide comprises two CD47-binding SIRP IgV monomers, wherein the N-terminal SIRP IgV monomer comprises a mutation of K53H, R69H, Q52H, K68H, Q52H+K68H, K53H+K68H, R69H+K68H, K53H+Q52H, R69H+Q52H, K53H+K68H+Q52H, and R69H+K68H+Q52H, and the C-terminal SIRP IgV monomer is a wild type SIRP IgV. In certain embodiments, the SIRP IgV multimer polypeptide comprises two CD47-binding SIRP IgV monomers, wherein the N-terminal SIRP IgV monomer is a wild type SIRP IgV monomer, and the C-terminal SIRP IgV monomer comprises a mutation of K53H, R69H, Q52H, K68H, Q52H+K68H, K53H+K68H, R69H+K68H, K53H+Q52H, R69H+Q52H, K53H+K68H+Q52H, and R69H+K68H+Q52H.

In some embodiments, the SIRP IgV multimer polypeptide comprises three CD47-binding SIRP IgV variant monomers, wherein the three monomers comprise the same mutation. In certain embodiments, the SIRP IgV multimer polypeptide comprises three CD47-binding SIRP IgV variant monomers comprising the same mutation selected from the group consisting of K53H, R69H, Q52H, K68H and Q52H+K68H. In certain embodiments, the SIRP IgV multimer polypeptide comprises three CD47-binding SIRP IgV variant monomers comprising the same mutation, wherein the mutation from the N-terminal monomer to the middle monomer to the C-terminal monomer of the SIRP IgV multimer polypeptide comprises a substitution of K53H-K53H-K53H (i.e. the N-terminal monomer comprising K53H, the middle monomer comprising K53H, and the C-terminal monomer comprising K53H), R69H-R69H-R69H, Q52H-Q52H-Q52H, K68H-K68H-K68H, or Q52H+K68H-Q52H+K68H-Q52H+K68H, wherein the “+” indicates concurrent mutation in a single SIRP IgV monomer, while the “−” indicates separate mutation of two neighboring SIRP IgV monomers serially linked from the N-terminal to the C-terminal, wherein the mutation from the left to the right of the “−” indicates the mutation from the N-terminal to the C-terminal. In certain embodiments, the SIRP IgV multimer polypeptide comprises three CD47-binding SIRP IgV variant monomers comprising the same mutation selected from the group consisting of K53H, R69H, Q52H, K68H and Q52H+K68H, wherein the three monomers comprise one or more the same additional mutation. In further embodiments, the additional mutation comprises I31E/L31E or I31D/L31D. In some embodiments, the SIRP IgV multimer polypeptide comprises three CD47-binding SIRP IgV variant monomers, wherein the three monomers comprise different mutation. In certain embodiments, the SIRP IgV multimer polypeptide comprises three CD47-binding SIRP IgV variant monomers comprising different mutation, wherein the mutation from the N-terminal monomer to the middle monomer to the C-terminal monomer of the SIRP IgV multimer polypeptide comprises a substitution of K53H-K53H-R69H (i.e. the N-terminal monomer comprising K53H, the middle monomer comprising K53H, and the C-terminal monomer comprising R69H), R69H-K53H-K53H, K53H-R69H-K53H, K53H-R69H-R69H, R69H-R69H-K53H, R69H-K53H-R69H, K53H-K53H-Q52H, Q52H-K53H-K53H, K53H-Q52H-K53H, K53H-K53H-K68H, K68H-K53H-K53H, K53H-K69H-K68H, Q52H-R69H-R69H, R69H-R69H-Q52H, R69H-Q52H-R69H, K68H-R69H-R69H, R69H-R69H-K68H, or R69H-K68H-R69H. In certain embodiments, the SIRP IgV multimer polypeptide comprises three CD47-binding SIRP IgV variant monomers comprising different mutation, wherein the mutation from the N-terminal monomer to the middle monomer to the C-terminal monomer comprise a substitution of K53H-K53H-R69H (i.e. N-terminal monomer comprising K53H, middle monomer comprising K53H, and C-terminal monomer comprising R69H), R69H-K53H-K53H, K53H-R69H-K53H, K53H-R69H-R69H, R69H-R69H-K53H, R69H-K53H-R69H, K53H-K53H-Q52H, Q52H-K53H-K53H, K53H-Q52H-K53H, K53H-K53H-K68H, K68H-K53H-K53H, K53H-K68H-K53H, Q52H-69H-R69H, R69H-R69H-Q52H, R69H-Q52H-R69H, K68H-R69H-R69H, R69H-R69H-K68H, or R69H-K68H-R69H, and the N-terminal monomer, the middle monomer and/or the C-terminal monomer comprise one or more additional mutations, wherein the one or more additional mutations of the N-terminal monomer, the middle monomer and/or the C-terminal monomer are the same or different. In further embodiments, the additional mutation comprises I31E/L31E or I31D/L31D. In certain embodiments, the SIRP IgV multimer polypeptide comprises three CD47-binding SIRP IgV variant monomers, wherein only one of the three SIRP IgV monomers comprises a mutation of K53H, R69H, Q52H, K68H, Q52H+K68H, K53H+K68H, R69H+K68H, K53H+Q52H, R69H+Q52H, K53H+K68H+Q52H, and R69H+K68H+Q52H. In certain embodiments, the SIRP IgV multimer polypeptide comprises three CD47-binding SIRP IgV variant monomers, wherein only two of the three SIRP IgV monomers comprises a mutation of K53H, R69H, Q52H, K68H, Q52H+K68H, K53H+K68H, R69H+K68H, K53H+Q52H, R69H+Q52H, K53H+K68H+Q52H, and R69H+K68H+Q52H. In certain embodiments, the SIRP IgV multimer polypeptide comprises three CD47-binding SIRP IgV monomers, wherein two of the three SIRP IgV monomers comprises a mutation of K53H, R69H, Q52H, K68H, Q52H+K68H, K53H+K68H, R69H+K68H, K53H+Q52H, R69H+Q52H, K53H+K68H+Q52H, and R69H+K68H+Q52H, and the remaining SIRP IgV monomer is a wild type SIRP IgV monomer.

In some embodiments, the SIRP IgV multimer polypeptide comprises four CD47-binding SIRP IgV variant monomers, wherein the four monomers comprise the same mutation. In certain embodiments, the SIRP IgV multimer polypeptide comprises four CD47-binding SIRP IgV variant monomers comprising the same mutation selected from the group consisting of K53H, R69H, Q52H, K68H and Q52H+K68H. In certain embodiments, the SIRP IgV multimer polypeptide comprises four CD47-binding SIRP IgV variant monomers comprising the same mutation, wherein the mutation from the N-terminal monomer to the C-terminal monomer of the SIRP IgV multimer polypeptide serially comprises a substitution of K53H-K53H-K53H-K53H, R69H-R69H-R69H-R69H, Q52H-Q52H-Q52H-Q52H, K68H-K68H-K68H-K68H, or Q52H+K68H-Q52H+K68H-Q52H+K68H-Q52H+K68H. In certain embodiments, the SIRP IgV multimer polypeptide comprises four CD47-binding SIRP IgV variant monomers comprising the same mutation selected from the group consisting of K53H, R69H, Q52H, K68H and Q52H+K68H, wherein the four monomers comprise one or more the same additional mutation. In further embodiments, the additional mutation comprises I31E/L31E or I31D/L31D. In some embodiments, the SIRP IgV multimer polypeptide comprises four CD47-binding SIRP IgV variant monomers, wherein the four monomers comprise different mutation. In certain embodiments, the SIRP IgV multimer polypeptide comprises four CD47-binding SIRP IgV variant monomers comprising different mutation, wherein the mutation from the N-terminal monomer to the C-terminal monomer serially comprises a substitution of K53H-K53H-R69H-R69H, R69H-R69H-K53H-K53H, R69H-R69H-Q52H-Q52H, Q52H-Q52H-R69H-R69H, R69H-R69H-K68H-K68H, K68H-K68H-R69H-R69H, K53H-K53H-Q52H-Q52H, Q52H-Q52H-K53H-K53H, K53H-K53H-K68H-K68H, K68H-K68H-K53H-K53H, K53H-R69H-R69H-R69H, R69H-R69H-R69H-K53H, Q52H-R69H-R69H-R69H, R69H-R69H-R69H-Q52H, K68H-R69H-R69H-R69H, R69H-R69H-R69H-K68H, R69H-K53H-K53H-K53H, K53H-K53H-K53H-R69H, Q52H-K53H-K53H-K53H, K53H-K53H-K53H-Q52H, K68H-K53H-K53H-K53H, or K53H-K53H-K53H-K68H. In certain embodiments, the SIRP IgV multimer polypeptide comprises four CD47-binding SIRP IgV variant monomers comprising different mutation, wherein the mutation from the N-terminal monomer to the C-terminal monomer serially comprises a substitution of K53H-K53H-R69H-R69H, R69H-R69H-K53H-K53H, R69H-R69H-Q52H-Q52H, Q52H-Q52H-R69H-R69H, R69H-R69H-K68H-K68H, K68H-K68H-R69H-R69H, K53H-K53H-Q52H-Q52H, Q52H-Q52H-K53H-K53H, K53H-K53H-K68H-K68H, K68H-K68H-K53H-K53H, K53H-R69H-R69H-R69H, R69H-R69H-R69H-K53H, Q52H-R69H-R69H-R69H, R69H-R69H-R69H-Q52H, K68H-R69H-R69H-R69H, R69H-R69H-R69H-K68H, R69H-K53H-K53H-K53H, K53H-K53H-K53H-R69H, Q52H-K53H-K53H-K53H, K53H-K53H-K53H-Q52H, K68H-K53H-K53H-K53H, or K53H-K53H-K53H-K68H, and the N-terminal monomer, the second middle monomer, the third middle monomer and/or the C-terminal monomer comprise one or more additional mutations, wherein the one or more additional mutations of the N-terminal monomer, the second middle monomer, the third middle monomer and/or the C-terminal monomer are the same or different. In further embodiments, the additional mutation comprises I31E/L31E or I31D/L31D.

In some embodiments, a SIRP IgV multimer polypeptide comprising two SIRP IgV monomers comprising the same mutation selected from the group consisting of K53H, R69H, Q52H, K68H, I31E/L31E, I31D/L31D, Q52H+K68H, K53H+I31E/L31E, R69H+I31E/L31E, K68H+I31E/L31E, Q52H+I31E/L31E, Q52H+K68H+I31E/L31E, K53H+I31D/L31D, R69H+I31D/L31D, K68H+I31D/L31D, Q52H+I31D/L31D, and Q52H+K68H+I31D/L31D, exhibits higher binding to CD47 at an acidic pH than at physiological pH, whereas an otherwise identical SIRP IgV multimer polypeptide except without any of the mutation K53H, R69H, Q52H, K68H, I31E/L31E and I31D/L31D, does not exhibit higher binding to CD47 at an acidic pH than at physiological pH, and an otherwise identical SIRP IgV multimer polypeptide but wherein the mutation is replaced with a mutation of M72H, V33H or G97H instead does not exhibits higher binding to CD47 at an acidic pH than at physiological pH. In some embodiments, a SIRP IgV multimer polypeptide comprising three or four SIRP IgV monomers comprising the same mutation selected from the group consisting of K53H, R69H, I31D/L31D, K53H+K68H, R69H+K68H, K53H+Q52H, R69H+Q52H, K53H+I31E/L31E, and R69H+I31E/L31E, exhibits higher binding to CD47 at an acidic pH than at physiological pH, whereas an otherwise identical SIRP IgV multimer polypeptide except without any of the mutation K53H, R69H, Q52H, K68H, I31D/L31D and I31E/L31E does not exhibit higher binding to CD47 at an acidic pH than at physiological pH, and an otherwise identical SIRP IgV multimer polypeptide but wherein the mutation is replaced with a mutation of M72H, V33H or G97H instead does not exhibits higher binding to CD47 at an acidic pH than at physiological pH. In some embodiments, a SIRP IgV multimer polypeptide comprising two SIRP IgV monomers wherein the two SIRP IgV monomers comprising different mutations selected from the group consisting of K53H, R69H, Q52H, K68H, I31E/L31E, I31D/L31D, Q52H+K68H, K53H+I31E/L31E, R69H+I31E/L31E, K68H+I31E/L31E, Q52H+I31E/L31E, Q52H+K68H+I31E/L31E, K53H+I31D/L31D, R69H+I31D/L31D, K68H+I31D/L31D, Q52H+I31D/L31D, and Q52H+K68H+I31D/L31D, exhibits higher binding to CD47 at an acidic pH than at physiological pH. In some embodiments, a SIRP IgV multimer polypeptide comprising three or four SIRP IgV monomers wherein at least one of the monomers comprises a mutation of K53H, R69H, I31D/L31D, K53H+I31E/L31E or R69H+I31E/L31E, and at least two of the SIRP IgV monomers comprising a different mutation selected from the group consisting of K53H, R69H, Q52H, K68H, I31D/L31D, Q52H+K68H, K53H+I31E/L31E, R69H+I31E/L31E, K68H+I31E/L31E, Q52H+I31E/L31E, Q52H+K68H+I31E/L31E, K53H+I31D/L31D, R69H+I31D/L31D, K68H+31D/L31D, Q52H+I31D/L31D, and Q52H+K68H+I31D/L31D, exhibits higher binding to CD47 at an acidic pH than at physiological pH. In some embodiments, a SIRP IgV multimer polypeptide comprising an amino acid sequence selected from the group consisting of SEQ ID NO: 82 to 113, SEQ ID NO: 418 to 425 and SEQ ID NO: 430 to 435, exhibits higher binding to CD47 at an acidic pH than at physiological pH. In some embodiments, a SIRP IgV multimer polypeptide comprising an amino acid sequence selected from the group consisting of SEQ ID NO: 418 to 425 and SEQ ID NO: 430 to 435, exhibits higher binding to CD47 at an acidic pH than at physiological pH, whereas a SIRP IgV multimer polypeptide comprising an amino acid sequence selected from the group consisting of SEQ ID NO: 426 to 429 and SEQ ID NO: 436 to 439, does not exhibit higher binding to CD47 at an acidic pH than at physiological pH.

### Fusion Polypeptide

In one aspect, the present disclosure provides a fusion polypeptide comprising a CD47-binding SIRP IgV domain comprising one or more CD47-binding SIRP IgV monomers of present disclosure, and a non-CD47 binding domain. In some embodiments, the fusion polypeptide comprises a CD47-binding SIRP IgV domain comprising one, two, three, four, five, or six CD47-binding SIRP IgV monomers. In some embodiments, the fusion polypeptide comprises two, three, four or more CD47-binding SIRP IgV monomers, wherein the monomers comprise the same or different amino acid sequence and/or mutation, and wherein the fusion polypepide exhibits higher binding to CD47 at an acidic pH than at physiological pH. In some embodiments, the CD47-binding SIRP IgV domain of the fusion polypeptide comprises a SIRP IgV multimer polypeptide of present disclosure, wherein the SIRP IgV multimer polypeptide comprises two, three, four or more serially linked CD47-binding SIRP IgV monomers comprising the same or different amino acid sequence and/or mutation.

In some embodiments, one or more of the CD47-binding SIRP IgV monomers of the fusion polypeptide comprises the full extracellular domain (ECD) of the Signal-regulatory protein (SIRP) comprising a IgV fragment and two IgC fragments (SEQ ID NO: 114 to 117) of the Signal-regulatory protein (SIRP).

In some embodiments, the CD47-binding SIRP IgV domain of the fusion polypeptide comprises one CD47-binding SIRP IgV monomer. In certain embodiments, the CD47-binding SIRP IgV monomer comprises an amino acid sequence selected from the group consisting of SEQ ID NO: 3 to 81. In some embodiments, the CD47-binding SIRP IgV domain of the fusion polypeptide comprises two CD47-binding SIRP IgV monomers. In some embodiments, the CD47-binding SIRP IgV domain of the fusion polypeptide comprises a SIRP IgV multimer polypeptide of present disclosure comprising two CD47-binding SIRP IgV monomers. In certain embodiments, the SIRP IgV multimer polypeptide comprising two CD47-binding SIRP IgV monomers comprises an amino acid sequence selected from the group consisting of SEQ ID NO: 82 to 96 and SEQ ID NO: 418 to 425 In some embodiments, the CD47-binding SIRP IgV domain of the fusion polypeptide comprises three CD47-binding SIRP IgV monomers. In some embodiments, the CD47-binding SIRP IgV domain of the fusion polypeptide comprises a SIRP IgV multimer polypeptide of present disclosure comprising three CD47-binding SIRP IgV monomers. In certain embodiments, the SIRP IgV multimer polypeptide comprising three CD47-binding SIRP IgV monomers comprises an amino acid sequence selected from the group consisting of SEQ ID NO: 97 to 109 and SEQ ID NO: 430 to 435. In some embodiments, the CD47-binding SIRP IgV domain of the fusion polypeptide comprises four CD47-binding SIRP IgV monomers. In some embodiments, the CD47-binding SIRP IgV domain of the fusion polypeptide comprises a SIRP IgV multimer polypeptide of present disclosure comprising four CD47-binding SIRP IgV monomers. In certain embodiments, the SIRP IgV multimer polypeptide comprising four CD47-binding SIRP IgV monomers comprises an amino acid sequence selected from the group consisting of SEQ ID NO: 110 to 113. In some embodiments, the SIRP IgV monomers of the fusion polypeptide comprise the same amino acid sequence and/or mutation. In some embodiments, the SIRP IgV monomers of the fusion polypeptide comprise different amino acid sequence and/or mutation.

In some embodiments, the fusion polypeptide comprises one or more additional domain that binds to a non-CD47 antigen. In some embodiments, an SIRP IgV domain of the fusion polypeptide is linked to the N-terminal of a non-CD47 binding domain of the fusion polypeptide. In some embodiments, an SIRP IgV domain of the fusion polypeptide is linked to the C-terminal of a non-CD47 binding domain of the fusion polypeptide. In some embodiments, an SIRP IgV domain of the fusion polypeptide is linked at its N-terminal to the C-terminal of a first non-CD47 binding domain of the fusion polypeptide and is linked at its C-terminal to the N-terminal of a second non-CD47 binding domain of the fusion polypeptide.

In some embodiments, the fusion polypeptide comprises one CD47-binding SIRP IgV monomer and one or more additional domains that bind to a non-CD47 antigen. In some embodiments, the fusion polypeptide comprises one CD47-binding SIRP IgV monomer and an additional domain that does not bind to CD47, wherein the additional domain is a Fc region or CH2 region or CH3 region or fragment thereof, an antibody light chain or VL or CL or fragment thereof, or an antibody heavy chain or VH or CH1 or fragment thereof. In some embodiments, the fusion polypeptide comprises one CD47-binding SIRP IgV monomer and an additional domain that does not bind to CD47, wherein the additional domain is a VHH antibody or nanobody, an immunoglobulin new antigen receptor (IgNAR), or other single-chain domain antibody, D-Domain, nanofitin, adnectin, knottin, kunitz, avimer, affilin, ADAPT or an anticalin, DARPIN, affibody, affimer, centryin domain, fibronectin domain, D-Domain, nanofitin, adnectin, knottin, kunitz, avimer, affilin, ADAPT or other alternative scaffold known in the arts to function as antigen binding domain that binds to an antigen. In some embodiments, the fusion polypeptide comprises one CD47-binding SIRP IgV monomer and an additional domain that does not bind to CD47, wherein the additional domain is a peptide, a receptor or fragment thereof, a ligand or fragment thereof, a cytokine or fragmente thereof, or a chemokine or fragment thereof. In certain embodiments, the one SIRP IgV monomer of the fusion polypeptide described in this paragraph comprises an amino acid sequence selected from the group consisting of SEQ ID NO: 3 to 81. In some embodiments, the one SIRP IgV monomer of the fusion polypeptide described in this paragraph comprises a mutation of K53H. In some embodiments, the one SIRP IgV monomer of the fusion polypeptide described in this paragraph comprises a mutation of R69H. In some embodiments, the one SIRP IgV monomer of the fusion polypeptide described in this paragraph comprises a mutation of Q52H. In some embodiments, the one SIRP IgV monomer of the fusion polypeptide described in this paragraph comprises a mutation of K68H. In some embodiments, the one SIRP IgV monomer of the fusion polypeptide described in this paragraph comprises a mutation of Q52H+K68H.

In some embodiments, the fusion polypeptide comprises at least two CD47-binding SIRP IgV monomers, wherein the two SIRP IgV monomers are separated by an additional domain that does not bind to CD47, wherein one SIRP IgV monomer is linked to the N-terminal of the additional domain and the other SIRP IgV monomer is linked to the C-terminal of the additional domain. In some embodiments, the fusion polypeptide comprises at least two CD47-binding SIRP IgV monomers, wherein the two SIRP IgV monomers are separated by an additional domain that does not bind to CD47, wherein the additional domain is a Fc region or CH2 region or CH3 region or fragment thereof, an antibody light chain or VL or CL or fragment thereof, or an antibody heavy chain or VH or CH1 or fragment thereof. In some embodiments, the fusion polypeptide comprises at least two CD47-binding SIRP IgV monomers, wherein the two SIRP IgV monomers are separated by an additional domain that does not bind to CD47, wherein the additional domain is a VHH antibody or nanobody, an immunoglobulin new antigen receptor (IgNAR), or other single-chain domain antibody, D-Domain, nanofitin, adnectin, knottin, kunitz, avimer, affilin, ADAPT or an anticalin, DARPIN, affibody, affimer, centryin domain, fibronectin domain, D-Domain, nanofitin, adnectin, knottin, kunitz, avimer, affilin, ADAPT or other alternative scaffold known in the arts to function as antigen binding domain that binds to an antigen. In some embodiments, the fusion polypeptide comprises at least two CD47-binding SIRP IgV monomers, wherein the two SIRP IgV monomers are separated by an additional domain that does not bind to CD47, wherein the additional domain is a peptide, a receptor or fragment thereof, a ligand or fragment thereof, a cytokine or fragmente thereof, or a chemokine or fragment thereof. In some embodiments, the SIRP IgV monomers of the fusion polypeptide described in this paragraph comprise the same or different mutations selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H. In some embodiments, at least one of the SIRP IgV monomers of the fusion polypeptide described in this paragraph does not comprise a mutation selected from the group consisting of K53H, R69H, Q52H and K68H. In some embodiments, at least one of the SIRP IgV monomers of the fusion polypeptide described in this paragraph is wild type SIRP IgV monomer. In some embodiments, the SIRP IgV monomers of the fusion polypeptide described in this paragraph comprise a mutation of K53H. In some embodiments, the one SIRP IgV monomer of the fusion polypeptide described in this paragraph comprises a mutation of R69H. In some embodiments, the SIRP IgV monomers of the fusion polypeptide described in this paragraph comprise a mutation of Q52H. In some embodiments, the SIRP IgV monomers of the fusion polypeptide described in this paragraph comprise a mutation of K68H. In some embodiments, the SIRP IgV monomers of the fusion polypeptide described in this paragraph comprise a mutation of Q52H+K68H.

In some embodiments, the fusion polypeptide comprises a SIRP IgV multimer polypeptide of present disclosure comprising two, three, four, five or six SIRP IgV monomers and one or more additional domains that bind to a non-CD47 antigen. In some embodiments, the SIRP IgV monomers of the SIRP IgV multimer polypeptide of the fusion polypeptide described in this paragraph comprise the same or different mutations selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H. In some embodiments, at least one of the SIRP IgV monomers of the SIRP IgV multimer polypeptide of the fusion polypeptide described in this paragraph does not comprise a mutation selected from the group consisting of K53H, R69H, Q52H and K68H. In some embodiments, at least one of the SIRP IgV monomers of the SIRP IgV multimer polypeptide of the fusion polypeptide described in this paragraph is a wild type SIRP IgV monomer.

In some embodiments, the non-CD47 binding domain of the fusion polypeptide described in the paragraph to binds to a soluble antigen, an aggregate of antigen, a surface antigen on a cell, a surface antigen on a microbe, or a hapten. In some embodiments, the non-CD47 binding domain of the fusion polypeptide binds to an aggregate of antigen, wherein the aggregate of antigen comprises aggregate of proteins and/or lipids. In certain embodiment, the aggregate of antigen comprises aggregate of proteins selected from the group of proteins comprising B-amyloid (AB), amyloid fibril, serum amyloid P component (SAP), Tau, o-synuclein, immunoglobulin light chain, transthyretin, buntingtin, polyglutamine, apolipoprotein, amylin, B2-macroglobulin, insulin, superoxide dismutase (SOD), lysozyme and prion. In some embodiments, the non-CD47 binding domain of the fusion polypeptide binds to a surface antigen on a cell, wherein the cell comprises a diseased cell, an infected cell or an effector cell. In certain embodiments, the diseased cell is a cancer cell, a fibrotic cell, an atherosclerotic cell, an inflammatory cell or a senescent cell. In certain embodiments, the infected cell is a cell infected by bacterium, fungus, virus and/or parasite. In certain embodiments, the effector cell is a myeloid cell, a lymphocyte or a granulocyte. In certain embodiments, the effector cell is a macrophage, a monocyte, a dendritic cell, a B cell, a T cell, a NK cell, a NKT cell, an innate lymphoid cell (ILC), a neutrophil, and/or a mast cell. In certain embodiments, the non-CD47 binding domain of the fusion polypeptide binds to a surface antigen on a microbe, wherein the microbe is a bacterium, a fungus, a protozoa, or a virus. In certain embodiments, the non-CD47 binding domain of the fusion polypeptide binds to a hapten, wherein the hapten comprises a chelator that binds to a radionuclide or radiometal. In certain embodiments, the radionuclide comprises ²²⁵Ac, ²¹¹At, ²¹²Bi, ⁶²Cu, ⁶⁴Cu, ⁶⁷Cu, ⁶⁶Ga, ⁶⁷Ga, ⁶⁸Ga, ¹²³I, ¹²⁵I, ¹³¹O, ¹¹¹In, ¹⁷⁷Lu, ²¹²Pb, ¹⁸⁶Re, ¹⁸⁸Re, ⁴⁴⁵c, ¹⁴⁹Tb, ¹⁵²Tb, ¹⁵⁵Tb, ¹⁶¹Tb, ⁹⁰Y or ⁸⁹Zr. In certain embodiments, the chelator is a macrocyclic chelator, an acyclic chelator or a hybrid chelator. In additional embodiments, the chelator is selected from the group consisting of 1,4,7,10-Tetraazacyclododecane-1,4,7,10-tetraacetic acid (DOTA), DOTATATE, DOTA-Bn, DO2A, Diethylenetriamene pentaacetate (DTPA), 6-amino-1,4-diazepine-triacetate (DATA), PCTA, 1,4,7-triazacyclononane-N,N′,N″-triacetic acid (NOTA), NOTP, TRAP, 1,4,7-Triazacyclononane (TACN), 6-amino-6-methylperhydro-1,4-diazepinetetraacetic acid (AAZTA), H4pypa, H2dedpa, H2CHXhox, H2box, HBED and DFO, as described in (Sneddon et al., Curr Opin Chem Biol, 2021). In certain embodiments, the hapten comprises a hapten peptide histamine-succinyl-glycine (HSG).

In some embodiments, the non-CD47 binding domain of the fusion polypeptide described in the paragraph to binds to one or more antigens selected from the group consisting of Amyloid Beta, Amyloid fibril, SAP, CD38, SLAMF7, BCMA, GPRC5D, FcRH5, CD138, CD56, CD74, CD26, CD46, CD19, CD20, CD22, CD30, CD33, CD37, CD70, CD117, CD79b, CD123, CD52, CD98, CD205, PD-L1, HER2, EGFR, VEGFR2, VEGFR1, VEGF, CD93, TGFβ, IL-6, IL-6R, IL-8, GDF-15, GFRAL, FGFR2, FGFR3, FGFR4, FGFR1, GD2, GD3, B7-H3, B7-H4, phosphatidylserine, DR5, DR4, DR3, TL1A, CD95 (Fas), TNFR1, TNFR2, CD24, CD31, CD61, CD200, SLAMF3, SLAMF4, HLA-G, HLA-E, HLA-DR, NKG2D ligands (MICA, MICB, ULBP1-6), CD1d-presented lipid antigen, P-Cadherin, N-Cadherin, E-Cadherin, VE-Cadherin, R-cadherin (CDH4), K-cadherin (CDH6), VE-cadherin (CDH5), H-cadherin (CDH13), OB-cadherin, LI-cadherin (CDH17), CDH11, CDH19, CLDN18.2, CLDN6, CLDN3, CLDN4, CLDN7, CLDN1, CLDN2, CLDN5, CLDN8, CLDN9, CLDN10, CLDN11, CLDN12, CLDN13, CLDN14, CLDN15, CLDN16, CLDN17, CLDN20, CLDN23, Connexin 25 (Cx25), Cx26, Cx30, Cx30.3, Cx31, Cx31.1, Cx32, Cx36, C37, Cx43, Cx45, Cx46, Cx50, Nectin-4, Nectin-3, Nectin-2, Nectin-1, Necl-5, Necl-2, Necl-1, Necl-3, Necl-4, CD44, CD44v6, ICAM-1 (CD54), ICAM-2 (CD102), ICAM-3 (CD50), VCAM-1, NCAM, L1CAM, EpCAM, ALCAM (CD66), MCAM, MAdCAM-1, CEACAM5, CEACAM1, CEACAM6, CEACAM4, CEACAM7, CEACAM19, CEACAM3, L-selectin, E-selectin, P-selectin, integrin α5β1, αvβ1, αvβ3, αvβ5, αvβ6, αvβ8, fibronectin extradomain-β, CTGF, Siglec ligands (sialoglycan), MUC1, MUC1-Tn, MUC5AC, MUC15, MUC16, MUC17, EphA1, EphA2, EphA3, EphA4, EphA5, EphA6, EphA7, EphA8, EphB1, EphB2, EphB3, EphB4, MSLN, CSPG4, CA-IX, DLL3, DLL-4, GPC-3, FRα, PSMA, PSCA, GPNMB, HER3, ROR1, ROR2, Axl, TROP2, EGFRvIII, PTK7, GRP78, IGF-1R, PD-L2, TAG-72, NaPi2b, GUCY2C, LY6G6D, PDGFR-α, c-Met, 5T4, AGS-16, ALK1, ANG-2, SSTR2, ENPP3, gpA33, SLC44A4, SLC34A2, SLC39A6, Notch 1, Notch 2, Notch 3, Notch 4, STEAP1, CCR4, CXCR4, CD71, CD166, DKK-1, LRRC15, FAP, Tenascin, GP75, CD175, CD175s, CD176, Lewis Y, SLeA, MST1R, tissue factor, carbonic anhydrase 6, guanylate cyclase-C, ETBR, LIV-1, RANKL, STRA6, UPK1B, VTCN1, LY6E, Binin-4, Tweak, TweakR, SLTRK6, TEM1, TCRα/β, TCRγ/δ, CD3, 4-1BB, CD28, ICOS, CD27, OX40, GITR, LIGHT, CD226, NKG2D, NKG2C, NKp30, NKp44, NKp46, NKp80, DNAM-1, 2B4, KIR2DS, KIR3DS, NTBA, CD84, Ly9, PD-1, CTLA-4, TIGIT, LAG3, CD73, CD39, TIM3, VISTA, CD96, B7-H3, TMIGD2, BTLA, CD160, CD200R, CCR8, CEACAM1, NKG2A, NKG2B, KIR2DL, KIR3DL, LILRB1, LAIR1, LAIR2, CD16, CD32, CD64, CD89, CD35, CR2, CR3, CR4, CRIg, CD40, CSF1R, SIPRa, PSGL-1, VSIG4, CLEVER-1, LILRA1, LILRA2, LILRA3, LILRA4, LILRA5, LILRA6, LILRB1, LILRB2, LILRB3, LILRB4, LILRB5, LAIR1, LAIR2, CD200R, TREM1, TREM2, CD91, SR-A (CD204), CD36, MARCO, SCARF-1, TIM1, TIM-4, BAI1, Stabilin-2, RAGE, CD300f, CD14, CD44, MerTK, Tyro3, Axl, chemokine receptors, cytokine receptors, C-type lectin receptors, lipoprotein receptors, Siglecs, Toll-like receptors (TLR), DOTA (tetraxetan), DTPA, and HSG. In certain embodiments, the chemokine receptor is selected from the group consisting of CCR1, CCR2, CCR5, CCR7, CX3CR1, CXCR3, CXCR4, CXCR5 and CXCR6. In certain embodiments, the cytokine receptor is selected from the group consisting of IL-2R, IL-15R, IL-7R, TNFα, IL-12R, IFNα, IFNβ and IFNγ. In certain embodiments, the C-type lectin receptor is selected from the group consisting of CD205, CD206, CD280, PLA2R, Dectin-1, Dectin-2, Dectin-3 (MCL/CLECSF8/CLEC4D), CLEC5A, Mincle, DC-SIGN, DC-SIGNR, DNGR-1 (CLEC9A), CLEC2, DCL-1 (CLEC13A), CLEC12B (MAH), MelLec (CLEC1A), LSECtin, LOX-1, BDCA-2, MGL (CLEC10A), CD93, Langerin, M1CL (CLEC12A) and DCIR. In certain embodiments, the lipoprotein receptor is selected from the group consisting of CD91, LOX-1 and LDLR. In certain embodiments, the Toll-like receptor is selected from the group consisting of TLR1, TLR2, TLR4, TLR5 and TLR6. In certain embodiments, the Siglecs is selected from the group consisting of Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15 and Siglec-16.

In some embodiments, the non-CD47 binding domain of the fusion polypeptide described in the paragraph to binds to two different antigens, with 1) one antigen selected from the group consisting of CD38, SLAMF7, BCMA, GPRC5D, FcRH5, CD138, CD56, CD74, CD26, CD46, CD19, CD20, CD22, CD30, CD33, CD37, CD70, CD117, CD79b, CD123, CD52, CD98, CD205, PD-L1, HER2, EGFR, VEGFR2, VEGFR1, VEGF, CD93, TGFβ, IL-6, IL-6R, IL-8, GDF-15, GFRAL, FGFR2, FGFR3, FGFR4, FGFR1, GD2, GD3, B7-H3, B7-H4, phosphatidylserine, DR5, DR4, DR3, TL1A, CD95 (Fas), TNFR1, TNFR2, CD24, CD31, CD61, CD200, SLAMF3, SLAMF4, HLA-G, HLA-E, HLA-DR, NKG2D ligands (MICA, MICB, ULBP1-6), CD1d-presented lipid antigen, P-Cadherin, N-Cadherin, E-Cadherin, VE-Cadherin, R-cadherin (CDH4), K-cadherin (CDH6), VE-cadherin (CDH5), H-cadherin (CDH13), OB-cadherin, LI-cadherin (CDH17), CDH11, CDH19, CLDN18.2, CLDN6, CLDN3, CLDN4, CLDN7, CLDN1, CLDN2, CLDN5, CLDN8, CLDN9, CLDN10, CLDN11, CLDN12, CLDN13, CLDN14, CLDN15, CLDN16, CLDN17, CLDN20, CLDN23, Connexin 25 (Cx25), Cx26, Cx30, Cx30.3, Cx31, Cx31.1, Cx32, Cx36, C37, Cx43, Cx45, Cx46, Cx50, Nectin-4, Nectin-3, Nectin-2, Nectin-1, Necl-5, Necl-2, Necl-1, Necl-3, Necl-4, CD44, CD44v6, ICAM-1 (CD54), ICAM-2 (CD102), ICAM-3 (CD50), VCAM-1, NCAM, L1CAM, EpCAM, ALCAM (CD66), MCAM, MAdCAM-1, CEACAM5, CEACAM1, CEACAM6, CEACAM4, CEACAM7, CEACAM19, CEACAM3, L-selectin, E-selectin, P-selectin, integrin α5β1, αvβ1, avβ3, αvβ5, αvβ6, αvβ8, fibronectin extradomain-β, CTGF, Siglec ligands (sialoglycan), MUC1, MUC1-Tn, MUC5AC, MUC15, MUC16, MUC17, EphA1, EphA2, EphA3, EphA4, EphA5, EphA6, EphA7, EphA8, EphB1, EphB2, EphB3, EphB4, MSLN, CSPG4, CA-IX, DLL3, DLL-4, GPC-3, FRα, PSMA, PSCA, GPNMB, HER3, ROR1, ROR2, Axl, TROP2, EGFRvIII, PTK7, GRP78, IGF-1R, PD-L2, TAG-72, NaPi2b, GUCY2C, LY6G6D, PDGFR-α, c-Met, 5T4, AGS-16, ALK1, ANG-2, SSTR2, ENPP3, gpA33, SLC44A4, SLC34A2, SLC39A6, Notch 1, Notch 2, Notch 3, Notch 4, STEAP1, CCR4, CXCR4, CD71, CD166, DKK-1, LRRC15, FAP, Tenascin, GP75, CD175, CD175s, CD176, Lewis Y, SLeA, MST1R, tissue factor, carbonic anhydrase 6, guanylate cyclase-C, ETBR, LIV-1, RANKL, STRA6, UPK1B, VTCN1, LY6E, Binin-4, Tweak, TweakR, SLTRK6 and TEM1, and 2) the other antigen selected from the group consisting of TCRα/β, TCRγ/δ, CD3, 4-1BB, CD28, ICOS, CD27, CD70, OX40, GITR, LIGHT, CD226, NKG2D, NKG2C, NKp30, NKp44, NKp46, NKp80, DNAM-1, 2B4, KIR2DS, KIR3DS, NTBA, CD84, Ly9, PD-1, CTLA-4, TIGIT, LAG3, CD73, CD39, TIM3, VISTA, CD96, B7-H3, TMIGD2, BTLA, CD160, CD200R, CCR8, CEACAM1, NKG2A, NKG2B, KIR2DL, KIR3DL, LILRB1, LAIR1, LAIR2, CD16, CD32, CD64, CD89, CD35, CR2, CR3, CR4, CRIg, CD40, CSF1R, SIPRa, PSGL-1, VSIG4, CLEVER-1, LILRA1, LILRA2, LILRA3, LILRA4, LILRA5, LILRA6, LILRB1, LILRB2, LILRB3, LILRB4, LILRB5, LAIR1, LAIR2, CD200R, TREM1, TREM2, CD91, SR-A (CD204), CD36, MARCO, SCARF-1, TIM1, TIM-4, BAI1, Stabilin-2, RAGE, CD300f, CD14, CD44, MerTK, Tyro3, Axl, chemokine receptors, CCR1, CCR2, CCR5, CCR7, CX3CR1, CXCR3, CXCR4, CXCR5, CXCR6, cytokine receptors, IL-2R, IL-15R, IL-7R, TNFα, IL-12R, IFNα, IFNβ, IFNγ, C-type lectin receptors, CD205, CD206, CD280, PLA2R, Dectin-1, Dectin-2, Dectin-3 (MCL), CLEC5A, Mincle, DC-SIGN, DC-SIGNR, DNGR-1 (CLEC9A), CLEC2, DCL-1 (CLEC13A), CLEC12B (MAH), MelLec (CLEC1A), LSECtin, LOX-1, BDCA-2, MGL (CLEC10A), CD93, Langerin, M1CL (CLEC12A), DCIR, lipoprotein receptors, CD91, LOX-1, LDLR, Toll-like receptors (TLR), TLR1, TLR2, TLR4, TLR5, TLR6, Siglecs, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, DOTA (tetraxetan), DTPA (diethylenetriaminepentaacetic acid), and HSG (histamine-succinyl-glycine).

In some embodiments, the non-CD47 binding domain of the fusion polypeptide described in the paragraph [257] to [266] comprises a peptide or polypeptide with antigen-binding, a ligand or fragment thereof with receptor binding, a receptor or fragment thereof with ligand binding, a heavy chain variable domain (VH), a light chain variable domain (VL), a single chain fragment variable (scFv) comprising a VH and a VL, a single chain Fab domain (scFab), a VHH antibody or nanobody, a VH single chain antibody (VH dAb) or a VL single chain antibody (VL dAb), an immunoglobulin new antigen receptor (IgNAR), or other single-chain domain antibody, D-Domain, nanofitin, adnectin, knottin, kunitz, avimer, affilin, ADAPT or an anticalin, DARPIN, affibody, affimer, centryin domain, fibronectin domain, D-Domain, nanofitin, adnectin, knottin, kunitz, avimer, affilin, ADAPT or other alternative scaffold known in the arts to function as antigen binding domain that binds to the non-CD47 antigen.

In some embodiments, the non-CD47 binding domain of the fusion polypeptide described in the paragraph to comprises an antibody domain or antigen-binding fragment thereof comprising a set of heavy chain variable (VH) and/or light chain variable (VL) complementarity determining region (CDR) sequences at least 30%, at least 40%, at least 50%, at least 60%, at least 70%, at least 75%, at least 80%, at least 85%, at least 90% or at least 95% identical to the set of VH and/or VL CDR sequences according to the IMGT numbering scheme (The Immunologist, 7, 132-136 (1999); Dev Comp Immunol. 2003 January; 27 (1): 55-77) of an antibody selected from a group of exemplary antibodies as set forth in Table 2 to 4. In some embodiments, the non-CD47 binding domain of the fusion polypeptide described in the paragraph to comprises a peptide, a receptor or fragment thereof, a ligand or fragment thereof, a cytokine or fragment thereof, or a chemokine or fragment thereof, or a growth factor or fragment thereof. In some embodiments, the non-CD47 binding domain of the fusion polypeptide comprises an amino acid sequence at least 30%, at least 40%, at least 50%, at least 60%, at least 70%, at least 75%, at least 80%, at least 85%, at least 90% or at least 95% identical to an amino acid sequence selected from a group of exemplary peptides and polypeptides as set forth in Table 5.

In some embodiments, the fusion polypeptide comprises a Fc region or its functional fragment thereof. The functional fragment comprises CH2 and/or CH3. The Fc region is selected from the group consisting of human IgG1, IgG2, IgG3, IgG4, IgA, IgM, IgE, and its variants. In some embodiments, the Fc region is a human IgG1 with wild-type effector function (SEQ ID NO: 135). In some embodiments, the Fc region is a human IgG1 with enhanced effector function, comprising mutations known in the arts to enhance the effector function of human IgG1 Fc. In certain embodiments, the Fc region is a human IgG1 Fe with enhanced antibody-dependent cellular phagocytosis (ADCP) function, comprising a mutation selected from the group consisting of G236A, S239D/1332E, G236A/S239D/1332E, S239D/A330L/1332E, G236A/A330L/1332E, G236A/S239D/A330L/1332E, and F243L/R292P/Y300L/V305I/P396L, according to the EU numbering scheme. In some embodiments, the Fc region is a human IgG1 with reduced or abolished effector function, comprising mutations known in the arts to reduce or abolish the effector function of human IgG1 Fc. In certain embodiments, the Fc region is a de-glycosylated human IgG1 with mutation of N279A, N297G, N2795, or N279Q (SEQ ID NO: 132), according to the EU numbering scheme. In certain embodiments, the Fc region is a human IgG1 Fe comprising mutation of L234A+L235A, or L234A+L235A+G237A, or L234A+L235A+P329G (e.g. SEQ ID NO: 133 to 134), according to the EU numbering scheme. In some embodiments, the Fc region is a human IgG4 with a mutation of S228P (SEQ ID NO: 130), according to the EU numbering scheme. In some embodiments, the Fe region is a human IgG2 (SEQ ID NO: 131).

In some embodiments, the fusion polypeptide comprises a CD47-binding SIRP IgV domain and a non-CD47 binding domain, wherein the CD47-binding SIRP IgV domain is linked to the N-terminal and/or the C-terminal of the non-CD47 binding domain of the fusion polypeptide. In some embodiments, the fusion polypeptide comprises a CD47-binding SIRP IgV domain and a Fc region, wherein the CD47-binding SIRP IgV domain is linked to the N-terminal and/or the C-terminal of the Fe region of the fusion polypeptide. In some embodiments, the fusion polypeptide comprises a CD47-binding SIRP IgV domain, a non-CD47 binding domain and a Fc region, wherein the non-CD47 binding domain is linked to the N-terminal of the Fc region, and the CD47-binding SIRP IgV domain is linked to the N-terminal of the non-CD47 binding domain and/or the C-terminal of the Fc region. In some embodiments, the fusion polypeptide comprises a CD47-binding SIRP IgV domain, a non-CD47 binding domain and a Fc region, wherein the CD47-binding SIRP IgV domain is linked to the N-terminal of the Fc region, and the non-CD47 binding domain is linked to the N-terminal of the CD47-binding SIRP IgV domain and/or the C-terminal of the Fe region. In some embodiments, the fusion polypeptide comprises a CD47-binding SIRP IgV domain, a non-CD47 binding domain and a Fc region, wherein the non-CD47 binding domain is linked to the C-terminal of the Fc region, and the CD47-binding SIRP IgV domain is linked to the N-terminal of the Fc region and/or the C-terminal of the non-CD47 binding domain. In some embodiments, the fusion polypeptide comprises a CD47-binding SIRP IgV domain, a non-CD47 binding domain and a Fc region, wherein the CD47-binding SIRP IgV domain is linked to the C-terminal of the Fe region, and the non-CD47 binding domain is linked to the N-terminal of the Fc region and/or the C-terminal of the CD47-binding SIRP IgV domain. In some embodiments, the fusion polypeptide comprises a CD47-binding SIRP IgV domain, a non-CD47 binding domain and a Fc region, wherein the non-CD47 binding domain is linked to the N-terminal and the C-terminal of the Fc region, and the CD47-binding SIRP IgV domain is linked to the N-terminal of the non-CD47 binding domain at the N-terminal of the Fe region and/or the C-terminal of the non-CD47 binding domain at the C-terminal of the Fc region. In some embodiments, the fusion polypeptide comprises a CD47-binding SIRP IgV domain, a non-CD47 binding domain and a Fc region, wherein the CD47-binding SIRP IgV domain is linked to the N-terminal and the C-terminal of the Fc region, and the non-CD47 binding domain is linked to the N-terminal of the CD47-binding SIRP IgV domain at the N-terminal of the Fc region and/or the C-terminal of the CD47-binding SIRP IgV domain at the C-terminal of the Fc region.

In some embodiments, the non-CD47 binding domain of the fusion polypeptide comprises an antibody domain comprising a heavy chain (VH-CH1-CH2-CH3) or a light chain (VL-CL), wherein the CD47-binding SIRP IgV domain is linked to the N-terminal and/or the C-terminal of the heavy chain or the light chain. In some embodiments, the non-CD47 binding domain of the fusion polypeptide comprises an antigen-binding fragment of antibody comprising a Fd chain (VH-CH1) or a light chain (VL-CL), wherein the CD47-binding SIRP IgV domain is linked to the N-terminal and/or the C-terminal of the Fd chain or light chain. In some embodiments, the non-CD47 binding domain of the fusion polypeptide comprises an antigen-binding fragment of antibody comprising a VH or a VL, wherein the CD47-binding SIRP IgV domain is linked to the N-terminal and/or the C-terminal of the VH or VL.

In some embodiments, the CD47-binding SIRP IgV monomers, the non-CD47 binding domains, and/or the Fc region of the fusion polypeptides are linked directly or through a linker. In certain embodiments, the linker comprises an amino acid sequence of (GGGGS)n, wherein n=1, 2, 3, 4, 5, 6, 7 or 8 (e.g. SEQ ID NO: 118 to 123). In certain embodiments, the linker comprises GGGGGGGGS (SEQ ID NO: 119), GGGGSGGGGSGGGGS (SEQ ID NO: 120), GGGGSGGGGSGGGGSGGGGS (SEQ ID NO: 121), of GGGGSGGGGSGGGGSGGGGSGGGGSGGGGS (SEQ ID NO: 123). In certain embodiments, the linker comprises GGGGSGGGGGGGGSGGGGS (SEQ ID NO: 121).

### Fusion Protein

In one aspect, the present disclosure provides a fusion protein comprising one said fusion polypeptide of present disclosure. In one aspect, the present disclosure provides a fusion protein comprising two or more said fusion polypeptides of present disclosure. In one aspect, the present disclosure provides a fusion protein comprising one, two or more said fusion polypeptides of present disclosure, wherein the fusion protein exhibits higher binding to CD47 at an acidic pH than at physiological pH. In some embodiments, a fusion protein comprises two or more said fusion polypeptides, wherein the two or more said fusion polypeptides comprise the same number of CD47-binding SIRP IgV monomers. In certain embodiments, a protein comprises two or more said fusion polypeptides, wherein the two or more fusion polypeptides comprise different number of CD47-binding SIRP IgV monomers. In certain embodiments, a protein comprises two or more said fusion polypeptides, wherein the composition of mutation of the CD47-binding SIRP IgV monomers of the two or more fusion polypeptides is the same. In certain embodiments, a protein comprises two or more said fusion polypeptides, wherein the composition of mutation of the CD47-binding SIRP IgV variant monomers of the two or more fusion polypeptides is different.

In some embodiments, a fusion protein comprising one or more said fusion polypeptides of present disclosure comprises in total one, two, three or four CD47-binding SIRP IgV monomers of present disclosure. In certain embodiments, a fusion protein comprising one or more said fusion polypeptides comprises one CD47-binding SIRP IgV monomer in total. In certain embodiments, a fusion protein comprising one or more said fusion polypeptides comprises two CD47-binding SIRP IgV monomers in total. In certain embodiments, a fusion protein comprising one or more said fusion polypeptides comprises three CD47-binding SIRP IgV monomers in total. In certain embodiments, a fusion protein comprising one or more said fusion polypeptides comprises four CD47-binding SIRP IgV monomers in total.

In some embodiments, a fusion protein comprising one or more said fusion polypeptides comprises three, four or more CD47-binding SIRP IgV monomers in total and a human IgG1 Fc with wild type or enhanced effector function, wherein the CD47-binding SIRP IgV monomers preferably comprise a substitution of K53H or R69H.

TABLE 2
List of exemplary non-CD47 binding antibodies targeting disease associated antigens

| Antibody | | patent or literature reference, or antibody | Antibody |
|---|---|---|---|
| target | Antibody name | INN (International Nonproprietary Names) | developer/vendor |
| FAP | sibrotuzumab (BIBH1) | WO1999057151A2 | Boehringer Ingelheim |
| | Simlukafusp alfa (RG7461) | WO2012020006A2 | Roche |
| | MP0310 | | Molecular Partners |
| | OS4 | J Biotech 92, 159-168 (2001) | |
| | scFv33, scFv36 | Mol Med 7, 461-469 (2001) | |
| | MFP5 | WO2007077173A1 | Boehringer Ingelheim |
| TL1A | PF-06480605 | Br J Clin Pharmacol. 2020 April; 86(4): 812-824 | Pfizer |
| | tulisokibart (PRA023) | INN12368 | Prometheus |
| | TEV-48574 (C03V) | MAbs. 2018 May/June; 10(4): 664-677 | Teva |
| CTGF | pamrevlumab (FG-3019) | INN10060 | Fibrogen |
| SAP | dezamizumab | | GSK |
| CD138 | Indatuximab | INN9486 | ImmunoGen |
| CD74 | Milatuzumab | INN8922 | Immunomedics |
| | STRO-001 | | Sutro Biopharma |
| CD26 | YS110 | Br J Cancer. 2017 Apr. 25; 116(9): 1126-1134 | Y's AC Co., Ltd |
| | huCD26mAb | Blood Cancer J. 2018 Oct. 22; 8(11): 99 | |
| CD46 | FOR46 | | Fortis Therapeutics |
| GRP78 | PAT-SM6 | | Patrys Ltd. |
| CD56 | Lorvotuzumab | INN9299 | ImmunoGen |
| CD52 | Alemtuzumab | | Millennium; Genzyme |
| CD93 | DC-6001, DCBY02 | | DynamiCure |
| | JS013 | | Junshi Biosciences |
| EGFRvIII | AMG595 | Mol Cancer Ther. 2015 July; 14(7): 1614-24. | Amgen |
| | humMR1 | Int Immunopharmacol. 2014 February; 18(2): 304-10 | |
| ROR2 | ozuriftamab | INN11947 | BioAtla |
| TweakR | enavatuzumab (PDL192) | INN9354 | AbbVie |
| CCR4 | mogamulizumab | INN9374 | Kyowa Hakko Kirin |
| GFRAL | NGM120 | | NGM Biopharma |
| TGFβ1 | livmoniplimab | INN11741 | AbbVie |
| | metelimumab | INN8087 | Genzyme |
| TGFβ2 | lerdelimumab | INN7882 | CAT |
| TGFβ1/2/3 | fresolimumab | INN9158 | Genzyme |
| CD24 | hG7-BM3 | CN107226866A | |
| | Humanized SWA11 | U.S. Pat. No. 8,614,301B2 | |
| | ATG-031 | | Antengene |
| | ALB9, HN-01, cG7-MICA | Int J Cancer. 2021 Feb. 1; 148(3): 546-559 | |
| CD200 | Samalizumab (ALXN-6000) | | Alexion/AstraZeneca |
| | TTI-CD200 | Br J Haematol. 2021 April; 193(1): 155-159 | Trillium Therapeutics |
| CD61 | RLYB212 | | Rallybio |
| Phosphatidyl- | Bavituximab | INN8734 | Peregrine Pharma |
| serine | 3G4, 2aG4, IN11, Hu3g4, 9d2 | Immunotargets Ther. 2018 Jan. 23; 7: 1-14 | |
| DR4 | mapatumumab | INN8635 | GlaxoSmithKline |
| | TR1419 | Front Mol Biosci. 2021 Dec. 20; 8: 756599. | |
| | m921/922, 4H6, 4G7, AY4, | Antibodies (Basel). 2017 Oct. 25; 6(4): 16 | |
| | TR1-mAbs | | |
| CD95 (Fas) | E09, E09_Y58W, E09-Y52aR, | Cell Death Differ. 2012 July; 19(7): 1187-95 | MedImmune |
| | E09-R52bS, EP12r_E01, | | |
| | EP4b_E03, EP5b_E05, | | |
| | EP6b_B01, DX2, SM1.1 | | |
| | R-125224 | Int Immunol. 2006 January; 18(1): 113-24 | |
| PTK7 | Cofetuzumab | INN10675 | Pfizer/AbbVie |
| | Clone 12C6 | WO2007067730A2 | Medarex |
| | Clone 3G8, 12C6a, 2E11, | WO2007067730A2 | Medarex |
| | Clone E1, E2, E3, R4, E5, E6, | Lau, E. (2013). | University of Toronto |
| | E7, E8, E9, E10, E11, E12, F01 | | |

| | |
|---|---|
| Amyloid | Bapineuzumab (3D6), Gantenerumab, Crenezumab (mC2) |
| Amyloid | birtamimab (NEOD001), CAEL-101 (11-1F4) |
| Fibril | |
| IL-6/IL-6R | tocilizumab, sarilumab, siltuximab, satralizumab, clazakizumab, levilimab, sirukumab, |
| | olokizumab, ziltivekimab, elsilimomab |
| CD38 | Daratumumab, Isatuximab (SAR650984), SAR442085, felzartamab (MOR202), mezagitamab (TAK- |
| | 079), modakafusp alfa (TAK-573), TAK-169, XmAb968/AMG424, GBR1342, CID-103/TSK011010, |
| | HexaBody-CD38 (GEN3014), CD38-SIFbody, IGM-2644, STI-6129, STI-5171, Y150 (Wuhan YZY |
| | Biopharma), VBI-001 |
| SLAM7 | Elotuzumab, azintuxizumab (ABBV-383), Luc63, Luc90 |
| BCMA | Belantamab, Teclistamab, hSG16.17 (SEA-BCMA), AMG420, BI40153, BI40174, BM-9B5, BM-6E7, |
| | MEDI2228, HDP-101, CC-93269, PF-06863135, REGN5458, TNB383B |
| GPRC5D | GC5B596, GPA0039, GPA0018 |
| FcRH5 | RG6160 (BFCR4350A), MFRF3266A(DFRF4539A), HuMA13G9 (Genentech) |
| IGF-1R | cixutumumab, dalotuzumab, figitumumab, ganitumab, istiratumab, lonigutamab, robatumumab, |
| | teprotumumab |
| CD20 | rituximab, ofatumumab, ocrelizumab, ublituximab, obinutuzumab, ocaratuzumab (AME-133v, |
| | LY2469298), ibritumomab (Zevalin), tositumomab (Bexxar), divozilimab, veltuzumab, |
| | zuberitamab, ripertamab, glofitamab, mosunetuzumab, odronextamab (REGN-1979), |
| | epcoritamab, plamotamab, imvotamab, , FBTA05 |
| CD19 | tafasitamab, inebilizumab, obexelimab, loncastuximab, denintuzumab, coltuximab, FMC63, |
| | blinatumomab, duvortuxizumab |
| CD22 | Epratuzumab, MOXETUMOMAB, INOTUZUMAB, BAY-1862864, Pinatuzumab, Suciraslimab, GTB- |
| | 1550, JNJ-75348780 |
| CD37 | Otlertuzumab, BI-836826, |
| CD30 | brentuximab, iratumumab, AFM13 |
| CD70 | cusatuzumab (ARGX-110), MDX-1411, SEA-CD70, vorsetuzumab, IMM40H, IMM40M, anti-CD70 |
| | nanobody No. 1, 2 and 3 to 24 (WO2022262100), anti-CD70 nanobody No. 1 to 14 |
| | (CN113292652A), anti-CD70 antibodies (U.S. Pat. No. 11,377,500B2), LD70, BR108, MP-0533, BMS-936561 |
| | (MDX-1203), SGN-CD70A, SGN-75, AMG172, ARX-305, PRO-1160, CTX130, ALLO-316, P-CD70- |
| | ALLO1, 4SCAR70, C-4-29, CAT-248 |
| PD-L1 | Atezolizumab (MPDL3280A), Durvalumab (MEDI4736), Avelumab (MSB0010718C), Sugemalimab |
| | (CS1001), Envafolimab (KN035), cosibelimab (CK-301), Socazolimab (STI1014), BMS-936559 (MDX- |
| | 1105), Geptanolimab (GB226), TQB 2450, SHR 1316, SHR-1701, LY-3434172, BGB-A333, RC98, |
| | GR1405, CX-072, CTX-8371, , INBRX-105, CDX 527, PM 8001, ABL501, FS222, FS118, AVA021, YBL- |
| | 013, IMM2510, IBI318, IBI323, IBI322, 6MW3211, SH009, BAT7104, SG12473, IMM2505, ATG-101, |
| | HLX301, Y101D, NM21-1480, PM1022, LP008, HB0028, SH006, GT90008, TST005, LY01019, LBL-024, |
| | QLF31907, BJ-005, BR102, Q-1802 |
| VEGFR2 | ramucirumab, alacizumab, olinvacimab, pulocimab, vulinacimab |
| VEGF | tarcocimab, varisacumab, brolucizumab, abicipar, IMC-1C11, faricimab, vanucizumab, dipacimab, |
| | navicixizumab, ivonescimab |
| EGFR | Cetuximab, Panitumumab, Nimotuzumab, Necitumumab, amivantamab, depatuxizumab, |
| | duligotuzumab, Futuximab (Sym004), Modotuximab, imgatuzumab, matuzumab, zalutumumab, |
| | tomuzotuximab, bafisontamab, Mab A13, ER414, ER2, ER78, ER79, GC1118 |
| HER2 | Trastuzuamb, Pertuzumab, Margetuximab, disitamab (RC48-0), anbenitamab (KN026), |
| | zanidatamab (ZW-25), coprelotamab (GB-221), ertumaxomab, fidasimtamab (IBI315), |
| | zenocutuzumab (MCLA-128), runimotamab, gancotamab (MM-302), timigutuzumab, cinrebafusp |
| | alfa (PRS-343), tezatabep matraxetan |
| HER3 | barecetamab, elgemtumab, patritumab, seribantumab, lumretuzumab, zenocutuzumab, |
| | duligotuzumab, izalontamab, istiratumab, MM-111 |
| GD2 | dinutuximab, dinutuximab beta, lorukafusp alfa, naxitamab, nivatrotamab, 14G2a, ME361, |
| | MORAb-028, TRBs07 |
| B7-H3 | enoblituzumab, ifinatamab, mirzotamab, obrindatamab (MGD009), omburtamab (8H9), |
| | vobramitamab, ATG-027, M30, Hu-M30, DS-5573a |
| ROR1 | zilovertamab, CS5001 (LCB71), huXBR1-402 (BI 3702025/NBE-002) |
| DLL3 | rovalpituzumab, tarlatamab, PT217, 13P9 (Phanes Tx) |
| FRa | farletuzumab, luveltamab, mirvetuximab |
| PSMA | acapatamab (AMG-160), pasotuxizumab (AMG-212), pelgifatamab, rosopatamab (huJ-591), |
| | voxalatamab, MOR209/ES414 |
| GPC-3 | codrituzumab (GC33), PPMX-T001, MDX-1414, TAK-102, HN3, YP7, GPC-3298306, ERY-974, B010-A, |
| | CM-350, BOXR-1030, HLX-63, LQ-102, CT-011, ECT-204, ABP-110, CT-017, YTGPC-3, IM-83, GB- |
| | 5011, Ori-CAR-001 |
| AXL | enapotamab, mecbotamab, tilvestamab, mipasetamab |
| GUCY2C | indusatumab, PF-07062119 |
| CA-IX | girentuximab, BAY 79-4620 |
| RANKL | denosumab, ALX-0141, IBI307, JMT103 |
| GDF-15 | visugromab, AZD8853, AV-380, ponsegromab |
| DR5 | conatumumab, drozitumab, lexatumumab, tigatuzumab, tilogotamab, benufutamab, INBRX-109, |
| | IGM-8444, Zaptuzumab, KTRM2, LaDR5, LBY135, mDRA6, WD1 |
| HLA-G | TTX-080, IVS-4001, JNJ-78306358, IMB-201, 4H84, MEM-G9, G233 |
| CD117 | CDX-0158, CDX-0159 (barzolvolimab), briquilimab (JSP191), eglatoprutug, MGTA-117 |

TABLE 3
List of exemplary non-CD47 binding antibodies targeting cell adhesion molecules

| Antibody | | patent or literature reference, or antibody INN | |
|---|---|---|---|
| target | Antibody name | (International Nonproprietary Names) | Antibody developer |
| P-Cadherin | TSP7, TSP7 VH-S77R, TSP11, | Sci Rep. 2017 Jan. 3; 7: 39518 | |
| | TSP5 | | |
| | PF-03732010 | PCT/IB2006/001053; | Pfizer |
| | | Clin Cancer Res. 2010 Nov. 1; 16(21): 5177-88 | |
| | PF-06671008 | US20160002357A1; | Pfizer |
| | | Antibodies (Basel). 2016 Mar. 4; 5(1): 6 | |
| | DART 35, DART 34, DART 154, | Antibodies (Basel). 2016 Mar. 4; 5(1): 6 | Pfizer |
| | DART 165, DART 177, DART 178, | | |
| | DART 179, DART 180, DART 281 | | |
| | FF-21101 | PCT/JP2010/057694 | FUJIFILM Toyama Chem |
| | PCA062 | PCT/IB2015/058801 | Novartis |
| | Oncotherapy Clone #6 | PCT/JP2007/054374 | |
| K-cadherin | DS-6000, G019, H01L02, | WO2016024195A1 | Daiichi Sankyo |
| | H02L02, H02L03, H04L02 | | |
| (CDH6) | HKT288, NOV0670 to NOV0720, | WO2016024195A1 | Novartis |
| | NOV0712 | | |
| LI-Cadherin | ARB202 | U.S. Pat. No. 11,207,419B2 | Arbele Bio |
| (CDH17) | BI-905711 | Cancer Res (2019) 79 (13_Supplement): 2051 | Boehringer Ingelheim |
| | PA-0661 | | Protein Alternatives |
| | CHM-2301, CHM-2101 | | Chimeric |
| | | | Therapeutics |
| CDH11 | SDP051/RO7123520 | U.S. Pat. No. 8,940,300B2 | Adheron Therapeutics |
| N-cadherin | GC4 | J Cell Sci. (2005) 118: 711-21; BMC Cancer. 2018 Oct. | |
| | | 1; 18(1): 939 | |
| | 1H7 | Nat Med. (2010) 16: 1414-20 | |
| | 2A9 | J Cell Biol. (2000) 151: 1193-206 | |
| E-cadherin | SHE78-7 | Mol Cancer Ther. 2004 February; 3(2): 149-59 | |
| VE-caderin | BV13, BV14, E4G10 | Front Oncol. 2019 Oct. 4; 9: 989 | |
| OB-cadherin | 2C7 | Front Oncol. 2019 Oct. 4; 9: 989 | |
| desmoglein 2 | JO-1, JO-4 | Sci Rep. 2019 Apr. 16; 9(1): 6169 | |
| Claudin 1 | OM-7D3-B3, 3A2, 7A5 | Drug Discov Today. 2016 October; 21(10): 1711-1718 | |
| | Humanized OM-7D3-B3 | U.S. Pat. No. 10,851,160B2 | |
| | 6F6 | Int. J. Mol. Sci. 2019, 20(16), 4016 | |
| Claudin 2 | 1A2 | Eur J Pharm Sci. 2018 May 30; 117: 161-167 | |
| Claudin 3 | h4G3 | Biomolecules. 2019 Dec. 28; 10(1): 51 | |
| | KM3907, 5A5 | Drug Discov Today. 2016 October; 21(10): 1711-1718 | |
| | KMK3953, IgGH6 | Int. J. Mol. Sci. 2019, 20(16), 4016 | |
| | ABN501 | Cancer Res (2022) 82 (12_Supplement): 311. | Abion |
| | MORAb-075 | Cancer Res (2014) 74 (19_Supplement): 673. | Morphotek/Esai |
| Claudin 4 | KM3900, KM3907, 5AS | Drug Discov Today. 2016 October; 21(10): 1711-1718 | |
| | humanized KM3900 | WO2008114733A1 | Kyowa Hakko Kirin Co. |
| | 5D12 | Pharmacol Res Perspect. 2016 Sep. 27; 4(5): e00266 | |
| | KM3934 | Int. J. Mol. Sci. 2019, 20(16), 4016 | |
| | MORAb-075 | Cancer Res (2014) 74 (19_Supplement): 673. | Morphotek/Esai |
| | 4D3 | Oncotarget. 2018 Dec. 21; 9(100): 37367-37378 | |
| Claudin 5 | RS, R2, 2B12 | Int. J. Mol. Sci. 2019, 20(16), 4016 | |
| CX43 | MAbE2Cx43 | Bull Exp Biol Med. 2014 August; 157(4): 510-5 | |
| | ALMB-0168 | | AlaMab Therapeutics |
| Nectin-4 | enfortumab | INN9821 | Seattle Genetics |
| | BA3361 | WO2021257525A1 | BioAtla |
| | SBT6290 | WO2021168274A1 | Silverback |
| | | | Therapeutics |
| | ETx-22 | | Emergence |
| | | | Therapeutics |
| | N41mab and its humanized | WO2017042210A1 | INSERM |
| | variants | | |
| | Clone 5M21, 42D20, 20M12, | WO2021213434A1 | Mabwell (Shanghai) |
| | 26G13, 29B12, 30J3, 7I18, 4E9, | | Bioscience |
| | 30L18, 9M13 | | |
| | Clone hNec4.01 to hNec4.11 | WO2019215728A1 | Nectin Therapeutics |
| | | | Ltd. |
| Nectin-1 | MAB2880 (clone #610835) | Oncotarget. 2017 Feb. 7; 8(6): 9717-9738 | R&D Systems |
| | MABT6 (clone R1.302) | Oncotarget. 2017 Feb. 7; 8(6): 9717-9738 | Millipore |
| Necl-5 | NTX1088 | | Nectin Therapeutics |
| (PVR) | | | |
| L1CAM | Ab417 | MAbs. 2016; 8(2): 414-25 | |
| ALCAM | CX-2009 | | CytomX Therapeutics |
| CEACAM6 | BAY1834942 | Oncoimmunology. 2021 Dec. 30; 11(1): 2008110 | Bayer AG |
| | NEO-201 | Cancer Biother Radiopharm. 2020 April; 35(3): | Precision Biologics, |
| | | 190-198 | Inc. |
| | h16C3 | US20130189268 | |
| | clone 5G2 | Sci Rep. 2016 Apr. 21; 6: 24823 | |
| CD44 | RG7356 (ARH460-16-2) | WO2014198843A1; Oncotarget. 2016 May | Roche |
| | | 31; 7(22): 32532-42; Cancer Res (2008) 68 | |
| | | (9_Supplement): 3975. | |
| | 5A4 | J Immunol. 1997 Jul. 15; 159(2): 919-25; PLoS One.2012; 7(3): e33142 | |
| | 8D2 | PLoS One. 2012; 7(3): e33142 | |
| | P4G9, P3D2, P3A7, P3G4 | PLoS One. 2021 Apr. 23; 16(4): e0250175. | |
| | H4C4 | Gastroenterology. 2014 April; 146(4): 1108-18 | |
| Selectin | SelG1 | Biomed Res Int. 2015; 2015: 417586 | Selexys |
| | | | Pharmaceuticals |
| α5β1 | Volociximab (M200) | Acta Pharm Sin B. 2021 September; 11(9): 2726-2737 | PDL BioPharma |
| αvß1, αvß3, | Intetumumab (CNTO 95) | Acta Pharm Sin B. 2021 September; 11(9): 2726-2737 | Centocor, Inc. |
| αvß5, αvß6 | | | |
| αvβ3, | Abciximab (c7E3) | Acta Pharm Sin B. 2021 September; 11(9): 2726-2737 | Eli Lilly |
| αIIbβ3, | | | |
| β1-integrins | P5D2 | Am J Pathol. 2001 December; 159(6): 2071-80 | |
| α5 integrins | P1D6 | Am J Pathol. 2001 December; 159(6): 2071-80 | |
| αv integrins | abituzumab | Mol Cancer Res. 2017 July; 15(7): 875-883 | Merck KgaA |
| MUC5AC | ensituximab | INN9300 | Neogenix Oncology |
| MUC17 | vepsitamab | INN11822 | Amgen |
| TAG-72 | anatumomab | INN7655 | |
| | M4 | | University of |
| | | | Saskatchew |
| FGFR1 | M6123 | WO2018095932A1 | Merck KGaA |
| | OM-RCA-01 | Invest New Drugs. 2013 December; 31(6): 1436-43 | |
| | D2 | Mol Cancer Res (2017) 15 (8): 1040-1050 | University of Wroclaw |
| | IMC-A1 | Am J Physiol Endocrinol Metab. 2007 | |
| | | March; 292(3): E964-76. | |
| | IMB-R1 | WO2012125124A1 | |
| FGFR2 | bemarituzumab (FPA144) | INN10681 | Five Prime/Amgen |
| | aprutumab | INN10382 | Bayer |
| | | | (Discontinued?) |
| | 4B9 (GP369), Hu4B9-65, | WO2011143318A2 | Aveo Pharmaceuticals |
| | Hu4B9-82, Hu4B9-83 | | |
| | DS-1123, FR2-14 and humanized | WO2013154206A1 | Daiichi Sankyo |
| | versions | | |
| | scFvF7, scFvD8, scFvG3, | PLoS One. 2018 Feb. 8; 13(2): e0192194 | |
| | scFvG9, scFvH6 | | |
| FGFR3 | vofatamab (R3mab) | INN10919 | Rainier Therapeutics |
| | PRO-001 | WO2007144893A2 | Fibron Ltd. |
| FGFR2/3 | PRO-007 | WO2007144893A2 | Fibron Ltd. |
| | 2B.1.3, 2B.1.3.10, 2B.1.3.12, | WO2016073789A2 | Genentech |
| | 2B.1.3.2, 2B.1.3.4, 2B.1.3.6, | | |
| | 2B.1.3.8, 2B.1.1, 2B.1.32, | | |
| | 2B.1.73, 2B.1.88, 2B.1.95 | | |
| FGFR4 | U3-1784 (Clone 9A5) | U.S. Pat. No. 9,284,379B2; Mol Cancer Ther. 2019 | Daiichi Sankyo |
| | | October; 18(10): 1832-1843 | |
| | hLD1.v22 | U.S. Pat. No. 9,266,955B2 | Genentech |
| | LD1 | PLoS One. 2012; 7(5): e36713 | Genentech |
| | 3A11 and humanized variants | WO2017049296A1 | US Department of HHS |
| | FGFR4-sdAb | Cancers (Basel). 2020 Nov. 10; 12(11): 3313 | University of Bern |
| EphA2 | DS-8895a | J Hematol Oncol. 2020 Aug. 18; 13(1): 114; | Daiichi Sankyo |
| | | J immunother Cancer. 2019 Aug. 14; 7(1): 219 | |
| | MEDI-547, MM-310, 3F2-3M, | J Hematol Oncol. 2020 Aug. 18; 13(1): 114 | AstraZeneca |
| | SHM16, EA1.2, EA2, B233 | | |
| | IGg28 | Expert Opin Investig Drugs. 2020 June; 29(6): 567-582 | |
| EphA3 | ifabotuzumab (KB004) | Expert Opin Investig Drugs. 2020 June; 29(6): 567-582 | Humanigen/KaloBios |
| EphA4 | NB39, NB53 | Expert Opin Investig Drugs. 2020 June; 29(6): 567-582 | |

| | |
|---|---|
| Claudin | zolbetuximab (IMAB-362), gresonitamab, osemitamab, ZL-1211, TJ-CD4B, 4F11E2, 72C1B6A3, 120B7B2, Hu8E5- |
| 18.2 | 21, AB011, RC-118, LM-102, LM-302, SKB315, CPO102, JS-012, SYSA-1801, Q-1802, DR-30303, KD-022, HBM- |
| | 1029, HLX-58, BNT-212, IBI-360, IBI-389, IBI-345, 5-E22 (humanized), PT886, CMG901, ABP-150, QL-1779, |
| | QLS-31905, SHR-A-1904, ASKB-589, BA-1105, NBL-015, M-108, YH005, SOT102, MIL93, BSI-077, ATG-022, |
| | LCAR-C18S, CT041 |
| Claudin 6 | ASP1650 (IMAB027), BNT211, TJ-C64B, clone 34927 |
| ICAM-1 | bersanlimab (BI-505), enlimomab, VBI-002 |
| EpCAM | adecatumumab, catumaxomab, citatuzumab, oportuzumab, edrecolomab, solitomab, tucotuzumab, |
| | UBS-54, VBI-003 |
| CEACAM5 | cergutuzumab, cibisatamab, labetuzumab, tusamitamab, PR1A3, humanized PR1A3, AMG-211, BDC-2034, |
| | clone 5G2, |
| (CEA) | MN-3, MN-15, NEO-201, 15-1-32, T84.66, BW431/26, CE25/B7, h16C3, COL-1, |
| CEACAM1 | CM24, TPP-3006 |
| CD44v6 | bivatuzumab, MAb U36, VFF18, VFF7 |
| αvβ3 | LM609, Etaracizumab (Abegrin), Vitaxin (MEDI-532) |
| αvβ8 | PF-06940434, MEDI8367, ADWA-11 |
| MUC1 | clivatuzumab, epitumomab, gatipotuzumab, sontuzumab, hCTM01 |
| MUC16 | oregovomab, sofituzumab, ubamatamab |
| MSLN | amatuximab, anetumab, inezetamab |

TABLE 4
List of exemplary non-CD47 binding antibodies targeting effector cell surface antigens

| Antibody | | patent or literature reference, or antibody | Antibody |
|---|---|---|---|
| Target | Antibody name | INN (International Nonproprietary Names) | developer/vendor |
| Dectin-1 | 2M24, 2M08, 2M12, 2M38 | WO2022077006A1 | Dren Bio |
| | 15E2, 2D8.2D4, 11B6.4, | WO2008118587A3 | |
| | 15F4.7, 9H7.6, 14D6.3 | | |
| | clone 259931 | Cat# MAB1859 | R&D Systems |
| | GE2 | Cat# Ab82888 | Abcam |
| | BD6 | Cat# MCA4662 | Bio-Rad |
| | MGD3 | WO2009150275A1 | |
| | aDectin-1 nanobody | CN113354735B | |
| Dectin-2 | BDC-3042 | WO2021222894A1 | Bolt Bio |
| CLEC5A | 1F7 | MAbs. 2022 January-December; 14(1): 2040083 | Genentech |
| | MAB2384 (clone #283834) | J Immunol Res. 2022 Feb. 25; 2022: 9926305 | R&D Systems |
| | MAB1639 (Clone # 226402) | | R&D Systems |
| CLEC10A | MLD-1 | J Biol Chem. 2002 Jun. 7; 277(23): 20686-93 | |
| (MGL) | clone 1G6.6, clone 18E4 | Immunobiology. 2006; 211(6-8): 577-85; | |
| | | Eur J Immunol. 2007 August; 37(8): 2075-81 | |
| CD205 | 3G9 | WO2009061996 | Celldex |
| | OBT076 | U.S. Pat. No. 10,081,682B2 | Oxford Biotherapeutics |
| CD206 | CDX-1307 | US20160355593A1 | Celldex |
| | anti-CD206 Clone 1, Clone 3 | US20110262348A1 | Vrije Universiteit |
| | | | Brussel VUB |
| | NbMMRm22.84, | US20200369773A1 | Celdara Medical |
| | NbMMRm5.38 | | |
| DC-SIGN | hD1V1G2/G4 (hD1) | Blood (2005) 106 (4): 1278-1285; | Alexion |
| | | U.S. Pat. No. 8,067,167B2 | Pharmaceuticals |
| CLEC9A | Humanized anti-CLEC9A | WO2022073062A1 | Monash University |
| | Ab1 | | |
| CD91 | clone II2C7 | Cancer Res. 2010 Jan. 1; 70(1): 277-87 | |
| CD89 | 2A9C11 | CN108659112B | Shanghai Tongji |
| | | | University |
| | HUMAb 14.1, 8D2 | WO2002064634A2 | Medarex |
| | 14A8 | J Immunol Res. 2016; 2016: 9425172 | Harvard Medical |
| | | | School |
| | A77 | Blood. 1997 Dec. 1; 90(11): 4485-92 | |
| CD36 | 1G04, 1G06, ONA-0-v1 | WO2021176424A1 | ONA Therapeutics |
| MerTK | RGX-019 | US20200255543A1 | Inspirna (Rgenix) |
| | M6, M19 | U.S. Pat. No. 10,221,248B2 | Rockefeller University |
| | 18G7 | WO2020214995A1; MAbs. 2020 January-December; | Genentech |
| | | 12(1): 1685832 | |
| | 14C3, 20F5 | MAbs. 2020 January-December; 12(1): 1685832 | Genentech |
| TREM1 | PY159 | US20210101983A1 | Pionyr Therapeutics |
| | CEL-383 | | Celsius Therapeutics |
| | TI-62, TI-63 | US20200131264A1 | Alector |
| MARCO | PY265 | WO2022109178A1 | Pionyr Therapeutics |
| CLEVER-1 | Bexmarilimab (FP-1305) | Faron Pharmaceuticals | |
| VSIG4 | VTX-1218 | | Verseau Therapeutics |
| LILRB1 | BND-22 (humanized 15G8), | U.S. Pat. No. 11,236,162B2 | Biond Bio |
| (ILT2) | 17F2, 19E3 | | |
| | NGM707 (Hz73D1.v1) | US20210355211A1 | NGM Bio |
| | 27F9, 47H6, 51A1, 64A12, | US20210355211A1 | NGM Bio |
| | 73C4, 73D1 | | |
| | AGEN1571 | | Agenus |
| | SAR444881 | | Sanofi |
| | iosH2 | | ImmunOs Therapeutics |
| | ATG-032 | | Antengene |
| | DM002 | | D2M Biotherapeutics |
| LAIR1/2 | SCTX001 | | Screen Therapeutics |
| CD200R1 | 23ME-00610 | WO2021243204A9 | 23andMe Holding |
| NKG2D | A49, A44 | WO2021041878A1 | Dragonfly Therapeutics |
| | AR3, AR6, AR11, AR18, | Oncoimmunology. 2020 Dec. 29; 10(1): 1854529 | Sanofi |
| | AR19, AR20, AR23 | | |
| | KYK-2.0 | J Mol Biol. 2008 Dec. 31; 384(5): 1143-56 | NCI |
| | tesnatilimab | INN11249 | Janssen |
| DNAM-1 | LY3435151 | WO2020023312A1 | Eli Lilly |
| | Clone NewE1 | Cancer Immunol Res. 2020 July; 8(7): 912-925 | |
| NKp46 | IPH6101 (SAR443579) | WO2017114694A1 | Innate Pharma |
| NKp30 | CTX-441, CTX-8573 | | Compass Therapeutics |

| | |
|---|---|
| CD16 | AFM13, AFM24, GTB-3550, DF1001, GMA161 |
| CD40 | sotigalimab, selicrelumab, dacetuzumab, mitazalimab, giloralimab, cifurtilimab, inezetamab, CDX- |
| | 1140, tecaginlimab, lucatumumab |
| PSGL-1 | VTX-0811 (SSGJ-617), neihulizumab (AbGn-168H), SelK2 (Selexys Pharma/Novartis), Leiolizumab |
| LILRB2 | MK-4830, JTX-8064, NGM707, IO-108, IVS-5001, ES009, CHS1000, CDX-585, ATG-032, DM002 |
| LILRB4 | IO-202, MK-0482, BND-35, NGM831, JTX-1484, SG2901, ATG-032 |
| LAIR1 | NC525, NGM438, IO-106 |
| MICL | Tepoditamab, ABL602, CLT-030, LB1910, ICG-136, CB012 |
| (CLEC12A) | |
| TREM2 | AL002, PY314, DNL919 |
| CSF1R | cabiralizumab, emactuzumab, axatilimab, LY-3022855 |
| CD3 | SP34, SP34 (humanized), OKT3, L2K, UCHT1, TR66, XR32, 26II6, BMA031, dafsolimab, visilizumab, |
| | foralumab, otelixizumab, teplizumab, tebentafusp, PF-06671008, TNB-383B, HPN328, tarlatamab, |
| | emirodatamab, acapatamab (AMG-160), alnuctamab, blinatumomab, catumaxomab, cevostamab, |
| | cibisatamab, duvortuxizumab, elranatamab, linvoseltamab, epcoritamab, mosunetuzumab, |
| | odronextamab, plamotamab, teclistamab, flotetuzumab, imvotamab, talquetamab, vixtimotamab |
| 4-1BB | urelumab, utomilumab, AGEN2373, LVGN6051, CTX-471, ATOR-1017, YH004, PE0116, ADG106, |
| | WBP3425, acasunlimab (GEN1046), GEN1042, cinrebafusp alfa, emfizatamab, MCLA-145, INBRX- |
| | 105, NM21-1480, ABL503, FS222, FS120, AMG506, STA551, CB307, APVO603, ALG-APV-527, ATG-101, |
| | TJ-C64B |
| OX40 | ivuxolimab (PF-04518600), tavolimab (MEDI0562), MEDI6469, GSK3174998, vonlerolizumab |
| | (MOXR0916), INCAGN01949, BMS-986178, revdofilimab (ABBV-368), cudarolimab (IBI-101), BGB- |
| | A445, INBRX-106, ATOR-1015, FS120, APVO603 |
| GITR | ragifilimab (INCAGN-01876), TRX-518, BMS-986156, AMG228, MK-4166, MK-1248, REGN-6569, |
| | GWN323, ASP1951, MEDI1873 |
| CD27 | Varlilumab, MK-5890 |
| CD28 | REGN5678, SAR442257, XmAb808, TGN1412, r2820 |
| ICOS | feladilimab, vopratelimab, alomfilimab, izuralimab |
| TIGIT | tiragolumab, domvanalimab, etigilimab, ociperlimab, tamgiblimab, vibostolimab, AGEN-1327, AGEN- |
| | 1777 |
| CTLA-4 | ipilimumab, tremelimumab, botensilimab (AGEN1181), zalifrelimab (AGEN1884), nurulimab, |
| | quavonlimab (MK-1308), REGN-4659, BA-3017, ONC-392, ADG-126, XTX-101, tuvonralimab, |
| | HBM4003, CS-1002, RIVAL-01, IMM-27M, JS-007, GI-101, BCD-217, YH001, BAT-4706, SKB-337, SI- |
| | B003, bavunalimab, cadonilimab, erfonrilimab, lorigerlimab, vudalimab, ADC-1015 |
| PD-1 | nivolumab, pembrolizumab, acrixolimab, balstilimab, budigalimab, camrelizumab, cemiplimab, |
| | cetrelimab, dostarlimab, ezabenlimab, finotonlimab, geptanolimab, iparomlimab, lodapolimab, |
| | nofazinlimab, penpulimab, peresolimab, pidilizumab, pimivalimab, prolgolimab, pucotenlimab, |
| | retifanlimab, rulonilimab, sasanlimab, sintilimab, rosnilimab, serplulimab, spartalizumab, |
| | tislelizumab, toripalimab, zimbirelimab, zeluvalimab, fidasimtamab, cadonilimab, ivonescimab, |
| | izuralimab, lorigerlimab, reozalimab, reozalimab, vudalimab, tebotelimab |
| LAG3 | relatlimab, encelimab, favezelimab, fianlimab, leramilimab, miptenalimab, INCAGN2385-101, |
| | Sym022, LBL-007, ES005, bavunalimab, tebotelimab, RG6139, EMB-02, CB213, FS118, IBI323, |
| | AVA021, YBL-013 |
| CD73 | oleclumab, uliledlimab, quisovalimab (dresbuxelimab, AK119), mupadolimab, BMS-986179, SRF373 |
| | (NVZ930), IPH5301, dalutrafusp alfa (AGEN-1423) |
| CD39 | SRF617, IPH5201, TTX-030, ES002 |
| NKG2A | Monalizumab, BMS-986315, HY-0102 |

TABLE 5
List of exemplary non-CD47 binding peptides and polypeptides

| Target | Peptide/polypeptide name and/or description | Reference |
|---|---|---|
| N-Cadherin | ADH-1 (cyclic pentapeptide) | Expert Opin Investig Drugs. 2007 April; 16(4): 451-65 |
| N-Cadherin | H-SWTLYTPSGQSK-NH 2 | Front Oncol. 2019 Oct. 4; 9: 989 |
| Claudin 3, 4, | Clostridium perfringens enterotoxin (CPE) | Mol Oncol. 2020 February; 14(2): 261-276 |
| 7, 8, 9 | | |
| Claudin 1, 3, | CPE-Mut1 (S313H) | Mol Oncol. 2020 February; 14(2): 261-276 |
| 4, 5, 6, 7, 9 | | |
| Claudin 1, 5 | CPE-Mut3 (S231R/S313H) | Mol Oncol. 2020 February; 14(2): 261-276 |
| Claudin 1, 6 | CPE-Mut4 (S305P/S307R/S313H) | Mol Oncol. 2020 February; 14(2): 261-276 |
| Claudin 5 | CPE-Mut6 (N218Q/Y306W/S313H) | Mol Oncol. 2020 February; 14(2): 261-276 |
| Claudin 3, 4, | CPE-Mut9 (Y306W/S313H) | Mol Oncol. 2020 February; 14(2): 261-276 |
| 5 | | |
| Claudin 4 | CPE-Mut10 (L254A/S256A/I258A/D284A) | Mol Oncol. 2020 February; 14(2): 261-276 |
| Nectin-4 | N4-P10, N4-P18, N4-P22 | Oncotarget. 2017 Feb. 7; 8(6): 9717-9738 |
| Nectin-1 | N1-P1, N1-P17, N1-P20, N1-P26 | Oncotarget. 2017 Feb. 7; 8(6): 9717-9738 |
| αvβ3 and αvβ5 | Cilengitide | Acta Pharm Sin B. 2021 September; 11(9): 2726-2737 |
| α5β1 | ATN-161 | Acta Pharm Sin B. 2021 September; 11(9): 2726-2737 |
| αvβ3 and α5β1 | AP25 | Acta Pharm Sin B. 2021 September; 11(9): 2726-2737; |
| | | Int J Med Sci 2019; 16(7): 1032-1041 |
| αvβ3 | HM-3 | Acta Pharm Sin B. 2021 September; 11(9): 2726-2737 |
| FGFR1 | F4_1, F4_2, F4_3 | Front Pharmacol. 2021; 12: 748936 |
| FGFR1 | FGF2-aa48-58, FGF2-aa59-68 | Cancers (Basel). 2020 October; 12(10): 2992 |
| CTGF | BLR-100, BLR-200 | Cells. 2020 Apr. 13; 9(4): 952 |
| TL1A | DR3 ECD and its variants A7, O6, H3, I12, G6, N8 | PLoS One. 2017 Mar. 9; 12(3): e0173460 |
| TGFβ | TGFβRII ECD | U.S. Pat. No. 9,676,863B2 |
| CD206 | RP-182, RP-183, RP-185 | WO2020210625A1 |
| TLRS | entolimod (CBLB502) | PLoS One. 2020; 15(2): e0227940 |
| TLR5 | GP532 | Commun Biol. 2021 Apr. 12; 4(1): 466. |
| CD91 | Calreticulin (full), Calreticulin (N-domain), | PLoS One. 2011; 6(3): e17886; US20200048371 |
| | Calreticulin (N-domain and P-domain) | |
| CD91 | SP16 (Peptide from SERPIN) | PLoS One. 2021 May 6; 16(5): e0247357 |
| Siglec ligands | Siglec 7 ECD | bioRxiv 2022.10.26.513931 |
| (sialoglycan) | | |
| Siglec ligands | Siglec 9 ECD | bioRxiv 2022.10.26.513931 |
| (sialoglycan) | | |
| Siglec ligands | Siglec 10 ECD | Biochem J. 2001 Apr. 15; 355(Pt 2): 489-497. |
| (CD24) | | |
| HLA-G | LILRB1 ECD, LILRB2 ECD, KIR2DL4 ECD | Int J Mol Sci. 2020 Nov. 17; 21(22): 8678 |
| NKG2D ligands | NKG2D ECD | U.S. Pat. No. 11,365,236B2 |
| NKG2D | MICA ECD, MICB ECD, ULBP1 ECD, ULBP2 ECD, | PLoS Pathog. 2010 Jan. 15; 6(1): e1000723 |
| | ULBP3 ECD, ULBP4 ECD, ULBP5 ECD, ULBP6 ECD | |
| CD28 | CD80 ECD | U.S. Pat. No. 11,098,103B2 |
| CD28 | CD86 ECD | WO2016168771A2 |
| ICOS | ICOSL ECD | WO2016168771A2 |

In some embodiments, a fusion protein comprising one or more said fusion polypeptides comprises a CD47-binding SIRP IgV domain, and a non-CD47 binding domain binding to one or more antigens selected from the group consisting of CD38, SLAMF7, BCMA, GPRC5D, FcRH5, CD138, CD56, CD74, CD26, CD46, CD19, CD20, CD22, CD30, CD33, CD37, CD70, CD117, CD79b, CD123, CD52, CD98, CD205, PD-L1, HER2, EGFR, VEGFR2, VEGFR1, VEGF, CD93, TGFβ, IL-6, IL-6R, IL-8, GDF-15, GFRAL, FGFR2, FGFR3, FGFR4, FGFR1, GD2, GD3, B7-H3, B7-H4, phosphatidylserine, DR5, DR4, DR3, TL1A, CD95 (Fas), TNFR1, TNFR2, CD24, CD31, CD61, CD200, SLAMF3, SLAMF4, HLA-G, HLA-E, HLA-DR, NKG2D ligands (MICA, MICB, ULBP1-6), CD1d-presented lipid antigen, P-Cadherin, N-Cadherin, E-Cadherin, VE-Cadherin, R-cadherin (CDH4), K-cadherin (CDH6), VE-cadherin (CDH5), H-cadherin (CDH13), OB-cadherin, LI-cadherin (CDH17), CDH11, CDH19, CLDN18.2, CLDN6, CLDN3, CLDN4, CLDN7, CLDN1, CLDN2, CLDN5, CLDN8, CLDN9, CLDN10, CLDN11, CLDN12, CLDN13, CLDN14, CLDN15, CLDN16, CLDN17, CLDN20, CLDN23, Connexin 25 (Cx25), Cx26, Cx30, Cx30.3, Cx31, Cx31.1, Cx32, Cx36, C37, Cx43, Cx45, Cx46, Cx50, Nectin-4, Nectin-3, Nectin-2, Nectin-1, Necl-5, Necl-2, Necl-1, Necl-3, Necl-4, CD44, CD44v6, ICAM-1 (CD54), ICAM-2 (CD102), ICAM-3 (CD50), VCAM-1, NCAM, L1CAM, EpCAM, ALCAM (CD66), MCAM, MAdCAM-1, CEACAM5, CEACAM1, CEACAM6, CEACAM4, CEACAM7, CEACAM19, CEACAM3, L-selectin, E-selectin, P-selectin, integrin α5B1, αvβ1, αvβ3, αvβ5, αvβ6, αvβ8, fibronectin extradomain-β, CTGF, Siglec ligands (sialoglycan), MUC1, MUC1-Tn, MUC5AC, MUC15, MUC16, MUC17, EphA1, EphA2, EphA3, EphA4, EphA5, EphA6, EphA7, EphA8, EphB1, EphB2, EphB3, EphB4, MSLN, CSPG4, CA-IX, DLL3, DLL-4, GPC-3, FRα, PSMA, PSCA, GPNMB, HER3, ROR1, ROR2, Axl, TROP2, EGFRvIII, PTK7, GRP78, IGF-1R, PD-L2, TAG-72, NaPi2b, GUCY2C, LY6G6D, PDGFR-α, c-Met, 5T4, AGS-16, ALK1, ANG-2, SSTR2, ENPP3, gpA33, SLC44A4, SLC34A2, SLC39A6, Notch 1, Notch 2, Notch 3, Notch 4, STEAP1, CCR4, CXCR4, CD71, CD166, DKK-1, LRRC15, FAP, Tenascin, GP75, CD175, CD175s, CD176, Lewis Y, SLeA, MST1R, tissue factor, carbonic anhydrase 6, guanylate cyclase-C, ETBR, LIV-1, RANKL, STRA6, UPK1B, VTCN1, LY6E, Binin-4, Tweak, TweakR, SLTRK6, TEM1, TCRα/β, TCRγ/δ, CD3, 4-1BB, CD28, ICOS, CD27, OX40, GITR, LIGHT, CD226, NKG2D, NKG2C, NKp30, NKp44, NKp46, NKp80, DNAM-1, 2B4, KIR2DS, KIR3DS, NTBA, CD84, Ly9, PD-1, CTLA-4, TIGIT, LAG3, CD73, CD39, TIM3, VISTA, CD96, B7-H3, TMIGD2, BTLA, CD160, CD200R, CCR8, CEACAM1, NKG2A, NKG2B, KIR2DL, KIR3DL, LILRB1, LAIR1, LAIR2, CD16, CD32, CD64, CD89, CD35, CR2, CR3, CR4, CRIg, CD40, CSF1R, SIPRa, PSGL-1, VSIG4, CLEVER-1, LILRA1, LILRA2, LILRA3, LILRA4, LILRA5, LILRA6, LILRB1, LILRB2, LILRB3, LILRB4, LILRB5, LAIR1, LAIR2, CD200R, TREM1, TREM2, CD91, SR-A (CD204), CD36, MARCO, SCARF-1, TIM1, TIM-4, BAIL, Stabilin-2, RAGE, CD300f, CD14, CD44, MerTK, Tyro3, Axl, chemokine receptors, cytokine receptors, C-type lectin receptors, lipoprotein receptors, Siglecs, Toll-like receptors (TLR), DOTA (tetraxetan), DTPA (diethylenetriaminepentaacetic acid), and HSG (histamine-succinyl-glycine). In certain embodiments, the chemokine receptor is selected from the group consisting of CCR1, CCR2, CCR5, CCR7, CX3CR1, CXCR3, CXCR4, CXCR5 and CXCR6. In certain embodiments, the cytokine receptor is selected from the group consisting of IL-2R, IL-15R, IL-7R, TNFα, IL-12R, IFNα, IFNβ and IFNγ. In certain embodiments, the C-type lectin receptor is selected from the group consisting of CD205, CD206, CD280, PLA2R, Dectin-1, Dectin-2, Dectin-3 (MCL/CLEC5F8/CLEC4D), CLEC5A, Mincle, DC-SIGN, DC-SIGNR, DNGR-1 (CLEC9A), CLEC2, DCL-1 (CLEC13A), CLEC12B (MAH), MelLec (CLEC1A), LSECtin, LOX-1, BDCA-2, MGL (CLEC10A), CD93, Langerin, M1CL (CLEC12A) and DCIR. In certain embodiments, the lipoprotein receptor is selected from the group consisting of CD91, LOX-1 and LDLR. In certain embodiments, the Toll-like receptor (TLR) is selected from the group consisting of TLR1, TLR2, TLR4, TLR5 and TLR6. In certain embodiments, the Siglecs is selected from the group consisting of Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15 and Siglec-16.

In some embodiments, a fusion protein comprising one or more said fusion polypeptides comprises a CD47-binding SIRP IgV domain, and non-CD47 binding domains binding to two different antigens, with 1) one antigen selected from the group consisting of CD38, SLAMF7, BCMA, GPRC5D, FcRH5, CD138, CD56, CD74, CD26, CD46, CD19, CD20, CD22, CD30, CD33, CD37, CD70, CD117, CD79b, CD123, CD52, CD98, CD205, PD-L1, HER2, EGFR, VEGFR2, VEGFR1, VEGF, CD93, TGFβ, IL-6, IL-6R, IL-8, GDF-15, GFRAL, FGFR2, FGFR3, FGFR4, FGFR1, GD2, GD3, B7-H3, B7-H4, phosphatidylserine, DR5, DR4, DR3, TL1A, CD95 (Fas), TNFR1, TNFR2, CD24, CD31, CD61, CD200, SLAMF3, SLAMF4, HLA-G, HLA-E, HLA-DR, NKG2D ligands (MICA, MICB, ULBP1-6), CD1d-presented lipid antigen, P-Cadherin, N-Cadherin, E-Cadherin, VE-Cadherin, R-cadherin (CDH4), K-cadherin (CDH6), VE-cadherin (CDH5), H-cadherin (CDH13), OB-cadherin, LI-cadherin (CDH17), CDH11, CDH19, CLDN18.2, CLDN6, CLDN3, CLDN4, CLDN7, CLDN1, CLDN2, CLDN5, CLDN8, CLDN9, CLDN1O, CLDN11, CLDN12, CLDN13, CLDN14, CLDN15, CLDN16, CLDN17, CLDN20, CLDN23, Connexin 25 (Cx25), Cx26, Cx30, Cx30.3, Cx31, Cx31.1, Cx32, Cx36, C37, Cx43, Cx45, Cx46, Cx50, Nectin-4, Nectin-3, Nectin-2, Nectin-1, Necl-5, Necl-2, Necl-1, Necl-3, Necl-4, CD44, CD44v6, ICAM-1 (CD54), ICAM-2 (CD102), ICAM-3 (CD50), VCAM-1, NCAM, L1CAM, EpCAM, ALCAM (CD66), MCAM, MAdCAM-1, CEACAM5, CEACAM1, CEACAM6, CEACAM4, CEACAM7, CEACAM19, CEACAM3, L-selectin, E-selectin, P-selectin, integrin α5β1, αvβ1, αvβ3, αvβ5, αvβ6, αvβ8, fibronectin extradomain-β, CTGF, Siglec ligands (sialoglycan), MUC1, MUC1-Tn, MUC5AC, MUC15, MUC16, MUC17, EphA1, EphA2, EphA3, EphA4, EphA5, EphA6, EphA7, EphA8, EphB1, EphB2, EphB3, EphB4, MSLN, CSPG4, CA-IX, DLL3, DLL-4, GPC-3, FRα, PSMA, PSCA, GPNMB, HER3, ROR1, ROR2, Axl, TROP2, EGFRvIII, PTK7, GRP78, IGF-1R, PD-L2, TAG-72, NaPi2b, GUCY2C, LY6G6D, PDGFR-α, c-Met, 5T4, AGS-16, ALK1, ANG-2, SSTR2, ENPP3, gpA33, SLC44A4, SLC34A2, SLC39A6, Notch 1, Notch 2, Notch 3, Notch 4, STEAP1, CCR4, CXCR4, CD71, CD166, DKK-1, LRRC15, FAP, Tenascin, GP75, CD175, CD175s, CD176, Lewis Y, SLeA, MST1R, tissue factor, carbonic anhydrase 6, guanylate cyclase-C, ETBR, LIV-1, RANKL, STRA6, UPK1B, VTCN1, LY6E, Binin-4, Tweak, TweakR, SLTRK6 and TEM1, and 2) the other antigen selected from the group consisting of TCRα/β, TCRγ/δ, CD3, 4-1BB, CD28, ICOS, CD27, CD70, OX40, GITR, LIGHT, CD226, NKG2D, NKG2C, NKp30, NKp44, NKp46, NKp80, DNAM-1, 2B4, KIR2DS, KIR3DS, NTBA, CD84, Ly9, PD-1, CTLA-4, TIGIT, LAG3, CD73, CD39, TIM3, VISTA, CD96, B7-H3, TMIGD2, BTLA, CD160, CD200R, CCR8, CEACAM1, NKG2A, NKG2B, KIR2DL, KIR3DL, LILRB1, LAIR1, LAIR2, CD16, CD32, CD64, CD89, CD35, CR2, CR3, CR4, CRIg, CD40, CSF1R, SIPRa, PSGL-1, VSIG4, CLEVER-1, LILRA1, LILRA2, LILRA3, LILRA4, LILRA5, LILRA6, LILRB1, LILRB2, LILRB3, LILRB4, LILRB5, LAIR1, LAIR2, CD200R, TREM1, TREM2, CD91, SR-A (CD204), CD36, MARCO, SCARF-1, TIM1, TIM-4, BAI1, Stabilin-2, RAGE, CD300f, CD14, CD44, MerTK, Tyro3, Axl, chemokine receptors, CCR1, CCR2, CCR5, CCR7, CX3CR1, CXCR3, CXCR4, CXCR5, CXCR6, cytokine receptors, IL-2R, IL-15R, IL-7R, TNFα, IL-12R, IFNα, IFNβ, IFNγ, C-type lectin receptors, CD205, CD206, CD280, PLA2R, Dectin-1, Dectin-2, Dectin-3 (MCL/CLEC5F8/CLEC4D), CLEC5A, Mincle, DC-SIGN, DC-SIGNR, DNGR-1 (CLEC9A), CLEC2, DCL-1 (CLEC13A), CLEC12B (MAH), MelLec (CLEC1A), LSECtin, LOX-1, BDCA-2, MGL (CLEC10A), CD93, Langerin, M1CL (CLEC12A), DCIR, lipoprotein receptors, CD91, LOX-1, LDLR, Toll-like receptors (TLR), TLR1, TLR2, TLR4, TLR5, TLR6, Siglecs, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, DOTA (tetraxetan), DTPA (diethylenetriaminepentaacetic acid), and HSG (histamine-succinyl-glycine).

In some embodiments, a fusion protein comprising one or more said fusion polypeptides comprises a CD47-binding SIRP IgV domain, and non-CD47 binding domains binding to two different antigens comprising one antigen associated with a diseased cell or tissue and one antigen as a surface receptor of an effector cell. In some embodiments, a fusion protein comprising one or more said fusion polypeptides comprises a CD47-binding SIRP IgV domain, and non-CD47 binding domains binding to two different antigens, with 1) one antigen selected from the group of disease associated antigens included in Table 2, and 2) the other antigen selected from the group effector cell surface receptor antigens included in Table 4. In some embodiments, a fusion protein comprising one or more said fusion polypeptides comprises a CD47-binding SIRP IgV domain, and non-CD47 binding domains binding to two different antigens, wherein the Fab domain binding to one antigen is derived from an effector cell surface receptor-binding antibody included in Table 4 and the Fab domain binding to the other antigen is derived from a disease associated antigen-binding antibody included in Table 2.

In some embodiments, a fusion protein comprising one or more said fusion polypeptides comprises a CD47-binding SIRP IgV domain, and non-CD47 binding domains binding to two different antigens comprising one cell adhesion molecule and one tumor associated antigen. In some embodiments, a fusion protein comprising one or more said fusion polypeptides comprises a CD47-binding SIRP IgV domain, and non-CD47 binding domains binding to two different antigens, with 1) one antigen selected from the group of cell adhesion molecules included in Table 3, and 2) the other antigen selected from the group tumor associated antigens included in Table 2. In some embodiments, a fusion protein comprising one or more said fusion polypeptides comprises a CD47-binding SIRP IgV domain, and non-CD47 binding domains binding to two different antigens, wherein the Fab domain binding to one antigen is derived from a cell adhesion molecule-binding antibody included in Table 3 and the Fab domain binding to the other antigen is derived from a disease associated antigen-binding antibody included in Table 2. In certain embodiments, a fusion protein comprising one or more said fusion polypeptides comprises a CD47-binding SIRP IgV domain, and non-CD47 binding domains binding to two different antigens, with 1) one antigen selected from the group consisting of cadberins, CDH3, CDH1, CDH2, CDH6, CDH17, nectins, Nectin-4, Nectin-1, Necl-5, integrins, α5 integrin, β1 integrin, claudins, CLDN4, CLDN1, Ig-superfamily CAMs, EpCAM, ICAM-1, carcinoembryonic antigen-related CAM, CEACAM5, CEACAM1, CEACAM6, and CD44, and 2) the other antigen selected from the group consisting of HER2, EGFR, VEGFR2, VEGFR1, VEGF, FGFR2, GD2, B7-H3, PD-L1, ROR1, CD70, CD117, CD38, SLAM7, BCMA, GPRC5D, FcRH5, CD30, CD20, CD19, CD22, CD37 and CD52. In certain embodiments, a fusion protein comprising one or more said fusion polypeptides comprises a CD47-binding SIRP IgV domain, and non-CD47 binding domains binding to two different antigens, with 1) one antigen selected from the group consisting of CDH3, Nectin-4, EpCAM, ICAM-1, CEACAM5, and CD44, and 2) the other antigen selected from the group consisting of HER2, EGFR, VEGFR2, VEGFR1, VEGF, FGFR2, GD2, B7-H3, PD-L1, ROR1, CD70, CD38 and SLAM7.

In some embodiments, a fusion protein comprising one or more said fusion polypeptides comprises a CD47-binding SIRP IgV domain and a non-CD47 binding antibody domain comprising a heavy chain (VH-CH1-CH2-CH3) and a light chain (VL-CL), wherein the CD47-binding SIRP IgV domain is linked to the N-terminal and/or the C-terminal of the heavy chain and/or the light chain. In some embodiments, a fusion protein comprising one or more said fusion polypeptides comprises a CD47-binding SIRP IgV domain and a non-CD47 binding antibody domain comprising a Fd chain (VH-CH1) and a light chain (VL-CL), wherein the CD47-binding SIRP IgV domain is linked to the N-terminal and/or the C-terminal of the Fd chain and/or light chain. In some embodiments, a fusion protein comprising one or more said fusion polypeptides comprises a CD47-binding SIRP IgV domain and a non-CD47 binding antibody domain comprising a VH and a VL, wherein the CD47-binding SIRP IgV domain is linked to the N-terminal and/or the C-terminal of the VH and/or VL. In certain embodiments, a fusion protein comprising one or more said fusion polypeptides comprises a CD47-binding SIRP IgV domain and a non-CD47 binding antibody domain comprising a heavy chain and a light chain, or a Fd chain and a light chain, or a VH and a VL, wherein the CD47-binding SIRP IgV domain is linked to the N-terminal of the light chain or VL. In certain embodiments, a fusion protein comprising one or more said fusion polypeptides comprises a CD47-binding SIRP IgV domain and a non-CD47 binding antibody domain comprising a heavy chain and a light chain, or a Fd chain and a light chain, or a VH and a VL, wherein the CD47-binding SIRP IgV domain is linked to the N-terminal of both the heavy chain and light chain, or both the Fd chain and light chain, or both the VH and VL.

In some embodiments, a fusion protein comprising one or more said fusion polypeptides comprises a CD47-binding SIRP IgV domain, wherein the CD47-binding SIRP IgV domain comprises one SIRP IgV monomer. In certain embodiments, the CD47-binding SIRP IgV domain comprises one SIRP IgV monomer comprising an amino acid sequence selected from the group consisting of SEQ ID NO: 3 to 81. In some embodiments, a fusion protein comprising one or more said fusion polypeptides comprises a CD47-binding SIRP IgV domain, wherein the CD47-binding SIRP IgV domain comprises two, three, four or more SIRP IgV monomers. In certain embodiments, a fusion protein comprising one or more said fusion polypeptides comprises a SIRP IgV multimer polypeptide of present disclosure comprising two, three or four SIRP IgV monomers. In certain embodiments, the SIRP IgV multimer polypeptide of the fusion protein comprises an amino acid sequence selected from the exemplary group consisting of SEQ ID NO: 82 to 113, SEQ ID NO: 418 to 425 and SEQ ID NO: 430 to 435.

In some embodiments, a fusion protein comprises two said fusion polypeptides comprising a Fe region, wherein the Fc region of the two fusion polypeptides form a homodimeric Fc. In certain embodiments, a fusion protein comprises two said fusion polypeptides comprising a Fe region, wherein the Fe region of the two fusion polypeptides comprise mutation that promote formation of heterodimeric Fc. In certain embodiments, a fusion protein comprises two said fusion polypeptides comprising a Fc region that forms a knobs-into-holes heterodimeric Fc, wherein the Fc region of one fusion polypeptide comprises mutation of T336W as the knob Fc half-chain, and the Fc region of the other fusion polypeptide comprises mutation of Y407V or T3665+L368A+Y407V as the hole Fc half-chain, according to the EU numbering scheme. In certain embodiments, a protein comprises two said fusion polypeptides comprising a Fe region that forms a knobs-into-holes heterodimeric Fc, wherein the Fc region of one fusion polypeptide comprises mutation of T336W+S354C as the knob Fe half-chain, and the Fc region of the other fusion polypeptide comprises mutation of Y407V+Y349C or T3665+L368A+Y407V+Y349C as the hole Fc half-chain, according to the EU numbering scheme. In certain embodiments, the knobs-into-holes heterodimeric Fc comprises a pair of Fc amino acid sequence selected from the group consisting of SEQ ID NO: 136:137, 138:139, 140:141 and 142:143, wherein the “:” indicates pairing of the two sequences from the left to right of the “:” symbol. In certain embodiments, the heterodimeric Fe comprises a human IgG1 Fe, comprising a pair of amino acid sequence of SEQ ID NO: 136:137.

In some embodiments, a fusion protein comprises one or more said fusion polypeptides is configured in one of the exemplary formats as set forth in FIGS. 2 to 12. In some embodiments, a Fc fusion protein comprising a said fusion polypeptide comprising a SIRP IgV domain, is configured in a format wherein the Fc fusion protein comprises: 1) a first polypeptide comprising a Fc region and a first SIRP IgV monomer, wherein the SIRP IgV monomer is linked to the N-terminal or C-terminal of the Fc region; and 2) a second polypeptide comprising a Fe region and a second SIRP IgV monomer, wherein the SIRP IgV monomer is linked to the N-terminal or C-terminal of the Fe region, and wherein the the Fc regions of the two polypeptides form a heterodimeric Fe. In certain embodiments, the Fc fusion protein is configured in FV-1 as set forth in FIG. 2, wherein the SIRP IgV monomer is linked to the N-terminal of the Fc region of the first and second polypeptide. In certain embodiments, the two SIRP IgV monomers of the Fe fusion protein comprise SIRPα, SIRPβ and/or SIRPγ IgV monomers. In certain embodiments, the first and second SIRP IgV monomers comprise different amino acid sequence and/or mutation. In certain embodiments, the first and second SIRP IgV monomers comprises two different mutation selected from the group consisting of K53H, R69H, Q52H, K68H and Q52H+K68H. In certain embodiments, the first and second SIRP IgV monomers comprise two different amino acid sequences selected from the group consisting of SEQ ID NO: 3 to 81. In certain embodiments, the first and second SIRP IgV monomers comprises a pair of two different mutation of K53HxR69H, K53HxQ52H, K53HxK68H, Q52HxR69H, or K68HxR69H, wherein the “x” indicates separate mutation in the first and second SIRP IgV monomers separately linked to the N-terminal of the two heterodimeric Fc chains. In certain embodiments, the first SIRP IgV monomer comprises a wild type SIRPα IgV and the second SIRP IgV monomer comprises a mutation of K53H, R69H, Q52H or K68H, or vice versa. In certain embodiments, the heterodimeric Fc comprises a human IgG1, human IgG4 or human IgG2 Fc. In certain embodiments, the heterodimeric Fe comprises a pair of Fe amino acid sequence selected from the group consisting of SEQ ID NO: 136:137, 138:139, 140:141 and 142:143, wherein the “:” indicates pairing of the two sequences from the left to right of the “:” symbol. In certain embodiments, the heterodimeric Fc comprises a human IgG1 Fe, comprising a pair of amino acid sequence of SEQ ID NO: 136:137.

In some embodiments, a Fc fusion protein comprising a said fusion polypeptide comprising a SIRP IgV domain, is configured in a format wherein one CD47-binding SIRP IgV monomer is linked to the N-terminal and/or C-terminal of an IgG Fc chain comprising hinge-CH2-CH3, wherein two Fc chains form a homodimeric Fc, and wherein the SIRP IgV monomer is a SIRPα, SIRPβ or SIRPγ IgV monomer. In certain embodiments, the Fc fusion protein is configured in FV-2 as set forth in FIG. 2, wherein the SIRP IgV domain is directly linked to the N-terminal of an IgG Fc chain. In certain embodiments, the Fc fusion protein is configured in FV-9 as set forth in FIG. 2, wherein the SIRP IgV domain is linked through a linker to the C-terminal of an IgG Fc chain. In certain embodiments, the SIRP IgV monomer comprises an amino acid sequence selected from the group consisting of SEQ ID NO: 3 to 81. In certain embodiments, the SIRP IgV monomer comprises a mutation of K53H. In certain embodiments, the SIRP IgV monomer comprises a mutation of R69H. In certain embodiments, the SIRP IgV monomer comprises a mutation of Q52H. In certain embodiments, the SIRP IgV monomer comprises a mutation of K68H. In certain embodiments, the SIRP IgV monomer comprises a mutation of Q52H+K68H. In certain embodiments, the SIRP IgV monomer comprises a mutation of I31H/L31H, I31Y/L31Y or I31W/L31W. In certain embodiments, the SIRP IgV monomer comprises a mutation of R59H. In some embodiments, a protein configured in FV-2 or FV-9 comprising two SIRP IgV monomers comprising the same mutation selected from the group consisting of K53H, R69H, Q52H, K68H, Q52H+K68H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a Fe-fusion protein configured in FV-2 comprising an amino acid sequence selected from the group consisting of SEQ ID NO: 301 to 304, 307, 309 to 312, 314 to 317, 319 to 324, and 326 to 336 exhibits higher binding to CD47 at an acidic pH than at physiological pH, whereas a Fc-fusion protein configured in FV-2 comprising an amino acid sequence selected from the group consisting of SEQ ID NO: 300, 308, 313, 318, 325, 306, 339 and 345, does not exhibit higher binding to CD47 at an acidic pH than at physiological pH.

In some embodiments, a Fc fusion protein comprising a said fusion polypeptide comprising a SIRP IgV domain, is configured in a format wherein a SIRP IgV multimer polypeptide of present disclosure comprising two SIRP IgV monomers is linked to the N-terminal and/or C-terminal of an IgG Fc chain comprising hinge-CH2-CH3, wherein two Fc chains form a homodimeric Fc, and wherein the two SIRP IgV monomers of the SIRP IgV multimer polypeptide comprise SIRPα, SIRPβ and/or SIRPγ IgV monomers. In certain embodiments, the Fe fusion protein is configured in FV-3 as set forth in FIG. 2, wherein the SIRP IgV multimer polypeptide is directly linked to the N-terminal of an IgG Fc chain. In some embodiments, the IgG Fc is a human IgG1 Fc. In certain embodiments, the two SIRP IgV monomers of the SIRP IgV multimer polypeptide comprise the same or different amino acid sequence and/or mutation. In certain embodiments, the two SIRP IgV monomers of the SIRP IgV multimer polypeptide comprise the same or different amino acid sequence selected from the group consisting of SEQ ID NO: 3 to 81. In certain embodiments, the mutation of the N-terminal monomer and the C-terminal monomer of the SIRP IgV multimer polypeptide comprise a substitution of K53H-K53H, R69H-R69H, K53H-R69H, R69H-K53H, Q52H-R69H, R69H-Q52H, K68H-R69H, R69H-K68H, Q52H-K53H, K53H-Q52H, K68H-K53H, K53H-K68H, Q52H-K68H, K68H-Q52H, Q52H+K68H-R69H, R69H-Q52H+K68H, Q52H+K68H-K53H, or K53H-Q52H+K68H, wherein the “+” indicates concurrent mutation in the same SIRP IgV monomer, while the “−” indicates separate mutation of two neighboring SIRP IgV monomers serially linked from the N-terminal to the C-terminal wherein the mutation from the left to the right of the “−” indicates the mutation from the N-terminal monomer to the C-terminal monomer. In some embodiments, the SIRP IgV multimer polypeptide comprises an amino acid sequence selected from the group consisting of SEQ ID NO: 82 to 96 and SEQ ID NO: 418 to 425. In some embodiments, the SIRP IgV multimer polypeptide comprises an amino acid sequence selected from the group consisting of SEQ ID NO: 418 to 425. In some embodiments, the SIRP IgV multimer polypeptide comprises an amino acid sequence selected from the group consisting of SEQ ID NO: 418 to 425, directly linked to the N-terminal of human IgG1 Fc (SEQ ID NO: 135). In some embodiments, a protein configured in FV-3 comprises a SIRP IgV multimer polypeptide comprising two SIRP IgV monomers comprising the same mutation selected from the group consisting of K53H, R69H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a protein configured in FV-3 comprises a SIRP IgV multimer polypeptide comprising two SIRP IgV monomers wherein the two SIRP IgV monomers comprise two different mutation selected from the group consisting of K53H, R69H, Q52H, K68H, Q52H+K68H, K53H+K68H, R69H+K68H, K53H+Q52H and R69H+Q52H, and at most one of the two SIRP IgV monomers comprises a mutation of Q52H, K68H or Q52H+K68H, exhibits higher binding to CD47 at an acidic pH than at physiological pH. In some embodiments, a protein configured in FV-3 comprises a SIRP IgV multimer polypeptide directly linked to the N-terminal of human IgG1 Fc (SEQ ID NO: 135) wherein the SIRP IgV multimer polypeptide comprises an amino acid sequence selected from the group consisting of SEQ ID NO: 418 to 425, exhibits higher binding to CD47 at an acidic pH than at physiological pH, whereas a protein in the same format comprising a SIRP IgV multimer polypeptide sequence selected from the group consisting of SEQ ID NO: 426 to 429, does not exhibit higher binding to CD47 at an acidic pH than at physiological pH.

In some embodiments, a Fc fusion protein comprising a said fusion polypeptide comprising a SIRP IgV domain, is configured in a format wherein the fusion protein comprises: 1) a partial heavy chain comprising CH1-hinge-CH2-CH3 from the N-terminal to C-terminal, 2) a partial light chain comprising CL domain, wherein the CL domain (SEQ ID NO: 124 or 125) pairs with the CH1 domain (SEQ ID NO: 126, 127 or 128) of the partial heavy chain with disulfide bond, 3) the Fe region of the two partial heavy chains form a homodimeric Fe, and 4) a SIRP IgV domain comprising one, two, three or more SIRP IgV monomers, wherein the SIRP IgV domain is linked preferably through a linker to the N-terminal of the partial light chain or the partial heavy chain. In certain embodiments, the fusion protein is configured in FV-8 as set forth in FIG. 2, wherein the SIRP IgV domain comprises one SIRP IgV monomer. In certain embodiment, the one SIRP monomer comprises a SIRPα, SIRPβ or SIRPγ IgV monomer. In certain embodiment, the SIRP monomer comprises an amino acid sequence selected from the group consisting of SEQ ID NO: 3 to 81. In certain embodiment, the SIRP monomer comprises a mutation of K53H. In certain embodiment, the SIRP monomer comprises a mutation of R69H. In certain embodiment, the SIRP monomer comprises a mutation of Q52H. In certain embodiment, the SIRP monomer comprises a mutation of K68H. In certain embodiment, the SIRP monomer comprises a mutation of Q52H+K68H. In certain embodiment, the SIRP monomer comprises a mutation of I31H/L31H, I31Y/L31Y, or I31W/L31W. In certain embodiment, the SIRP monomer comprises a mutation of R59H. In some embodiments, a protein configured in FV-8 comprising a SIRP IgV monomer comprising a mutation selected from the group consisting of K53H, R69H, Q52H, K68H, Q52H+K68H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a protein configured in FV-8 comprising a pair of partial heavy chain and partial light chain of SEQ ID NO: 440 and 454, or SEQ ID NO: 441 and 454, or SEQ ID NO: 442 and 454, or SEQ ID NO: 443 and 454, exhibits higher binding to CD47 at an acidic pH than at physiological pH, whereas a protein configured in FV-8 comprising a pair of partial heavy chain and partial light chain of SEQ ID NO: 446 and 454, or SEQ ID NO: 446 and 455, or SEQ ID NO: 444 and 454, or SEQ ID NO: 445 and 454, does not exhibit higher binding to CD47 at an acidic pH than at physiological pH. In certain embodiments, the fusion protein is configured in FV-4 as set forth in FIG. 2, wherein the SIRP IgV domain comprises a SIRP IgV multimer polypeptide comprising two SIRP IgV monomers. In certain embodiments, the two SIRP IgV monomers of the SIRP IgV multimer polypeptide comprise SIRPα, SIRPβ and/or SIRPγ IgV monomers. In certain embodiments, the two SIRP IgV monomers of the SIRP IgV multimer polypeptide comprise the same or different amino acid sequence and/or mutation. In certain embodiments, the two SIRP IgV monomers of the SIRP IgV multimer polypeptide comprise the same or different amino acid sequence selected from the group consisting of SEQ ID NO: 3 to 81. In certain embodiments, the mutation of the N-terminal monomer and the C-terminal monomer of the SIRP IgV multimer polypeptide comprise a substitution of K53H-K53H, R69H-R69H, K53H-R69H, R69H-K53H, Q52H-R69H, R69H-Q52H, K68H-R69H, R69H-K68H, Q52H-K53H, K53H-Q52H, K68H-K53H, K53H-K68H, Q52H-K68H, K68H-Q52H, Q52H+K68H-R69H. R69H-Q52H+K68H, Q52H+K68H-K53H, or K53H-Q52H+K68H, wherein the “+” indicates concurrent mutation in the same SIRP IgV monomer, while the “−” indicates separate mutation of two neighboring SIRP IgV monomers serially linked from the N-terminal to the C-terminal wherein the mutation from the left to the right of the “−” indicates the mutation from the N-terminal monomer to the C-terminal monomer. In some embodiments, the SIRP IgV multimer polypeptide comprises an amino acid sequence selected from the group consisting of SEQ ID NO: 82 to 96 and SEQ ID NO: 418 to 425. In some embodiments, the SIRP IgV multimer polypeptide comprises an amino acid sequence selected from the group consisting of SEQ ID NO: 418 to 425. In some embodiments, a protein configured in FV-4 comprises a SIRP IgV multimer polypeptide comprising two SIRP IgV monomers comprising the same mutation selected from the group consisting of K53H, R69H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a protein configured in FV-4 comprises a SIRP IgV multimer polypeptide comprising two SIRP IgV monomers wherein the two SIRP IgV monomers comprise two different mutation selected from the group consisting of K53H, R69H, Q52H, K68H, Q52H+K68H, K53H+K68H, R69H+K68H, K53H+Q52H and R69H+Q52H, and at most one of the two SIRP IgV monomers comprises a mutation of Q52H, K68H or Q52H+K68H, exhibits higher binding to CD47 at an acidic pH than at physiological pH. In some embodiments, a protein configured in FV-4 comprising a pair of partial heavy chain and partial light chain of SEQ ID NO: 447 and 454, or SEQ ID NO: 448 and 454, or SEQ ID NO: 449 and 454, or SEQ ID NO: 450 and 454, exhibits higher binding to CD47 at an acidic pH than at physiological pH, whereas a protein configured in FV-4 comprising a pair of partial heavy chain and partial light chain of SEQ ID NO: 451 and 454, or SEQ ID NO: 452 and 454, or SEQ ID NO: 453 and 454, or SEQ ID NO: 453 and 455, does not exhibit higher binding to CD47 at an acidic pH than at physiological pH.

In some embodiments, a Fc fusion protein comprising a said fusion polypeptide comprising a SIRP IgV domain, is configured in a format wherein the fusion protein comprises: 1) a partial heavy chain comprising CH1-binge-CH2-CH3 from the N-terminal to C-terminal, 2) a partial light chain comprising CL domain, wherein the CL domain (SEQ ID NO: 124 or 125) pairs with the CH1 domain (SEQ ID NO: 126, 127 or 128) of the partial heavy chain with disulfide bond, 3) the Fc region of the two partial heavy chains form a homodimeric Fc, and 4) a SIRP IgV domain comprising one, two, three or more SIRP IgV monomers, wherein the SIRP IgV domain is linked preferably through a linker to the N-terminal of the partial light chain and the partial heavy chain. In certain embodiments, the fusion protein is configured in FV-S as set forth in FIG. 2, wherein the SIRP IgV domain comprises a SIRP IgV monomer. In certain embodiments, the two SIRP IgV domains linked to the N-terminal of the partial heavy chain and the partial light chain comprise SIRPα, SIRPβ and/or SIRPγ IgV monomers. In certain embodiments, the two SIRP IgV domains linked to the N-terminal of the partial heavy chain and the partial light chain comprise the same or different amino acid sequence and/or mutation. In certain embodiments, the two SIRP IgV domains linked to the N-terminal of the partial heavy chain and the partial light chain comprise the same or different amino acid sequence selected from the group consisting of SEQ ID NO: 3 to 81. In certain embodiments, the two SIRP IgV domains comprise mutation of K53HxK53H, R69HxR69H, K53HxR69H, K53HxQ52H, K53HxK68H, Q52HxR69H, or K68HxR69H, wherein the “x” indicates separate mutation in the two separate SIRP IgV domains linked to the N-terminal of the partial heavy chain and partial light chain from the left to the right of the “x” symbol or to the N-terminal of the partial light chain and partial heavy chain from the left to the right of the “x” symbol. In some embodiments, a protein configured in FV-5 wherein the SIRP IgV monomers linked to the N-terminal of the partial heavy chain and partial light chain comprise the same mutation selected from the group consisting of K53H, R69H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a protein configured in FV-5 wherein the SIRP IgV monomers linked to the N-terminal of the partial heavy chain and partial light chain comprise two different mutations selected from the group consisting of K53H, R69H, K53H+K68H, R69H+K68H, K53H+Q52H and R69H+Q52H, exhibits higher binding to CD47 at an acidic pH than at physiological pH. In some embodiments, a protein configured in FV-5 comprising a pair of partial heavy chain and partial light chain of SEQ ID NO: 440 and 456, or SEQ ID NO: 441 and 457, or SEQ ID NO: 440 and 457, or SEQ ID NO: 441 and 456, or SEQ ID NO: 442 and 457, or SEQ ID NO: 443 and 457, exhibits higher binding to CD47 at an acidic pH than at physiological pH, whereas a protein configured in FV-S comprising a pair of partial heavy chain and partial light chain of SEQ ID NO: 444 and 458, or SEQ ID NO: 445 and 459, or SEQ ID NO: 446 and 460, or SEQ ID NO: 446 and 461, does not exhibit higher binding to CD47 at an acidic pH than at physiological pH.

In some embodiments, a Fc fusion protein comprising a said fusion polypeptide comprising a SIRP IgV domain, is configured in a format wherein a SIRP IgV multimer polypeptide of present disclosure comprising three SIRP IgV monomers is linked to the N-terminal or C-terminal of an IgG Fc chain comprising hinge-CH2-CH3 wherein two Fc chains form a homodimeric Fc, and wherein the six SIRP IgV monomers of the fusion protein comprise SIRPα, SIRPβ and/or SIRPγ IgV monomers. In certain embodiments, the Fc fusion protein is configured in FV-6 as set forth in FIG. 2, wherein the SIRP IgV multimer polypeptide is linked to the N-terminal of an IgG Fc chain. In certain embodiments, the three SIRP IgV monomers of the SIRP IgV multimer polypeptide comprise the same or different amino acid sequence and/or mutation. In certain embodiments, the three SIRP IgV monomers of the SIRP IgV multimer polypeptide comprise the same or different amino acid sequence selected from the group consisting of SEQ ID NO: 3 to 81. In certain embodiments, the three SIRP IgV monomers of the SIRP IgV multimer polypeptide from the N-terminal monomer to the middle monomer to the C-terminal monomer comprise a substitution of K53H-K53H-K53H, R69H-R69H-R69H, K53H-K53H-R69H, R69H-K53H-K53H, K53H-R69H-K53H, K53H-R69H-R69H, R69H-R69H-K53H, R69H-K53H-R69H, K53H-K68H-Q52H, Q52H-K53H-K53H, K53H-Q52H-K53H, K53H-K53H-K68H, K68H-K53H-K53H, K53H-K68H-K53H, Q52H-R69H-R69H, R69H-R69H-Q52H, R69H-Q52H-R69H, K68H-R69H-R69H, R69H-R69H-K68H, or R69H-K68H-R69H, wherein the “−” indicates separate mutation of two neighboring SIRP IgV monomers serially linked from the N-terminal to the C-terminal wherein the mutation from the left to the right of the “−” indicates the mutation from the N-terminal monomer to the C-terminal monomer. In some embodiments, the SIRP IgV multimer polypeptide comprises an amino acid sequence selected from the group consisting of SEQ ID NO: 97 to 109 and SEQ ID NO: 430 to 435. In some embodiments, the SIRP IgV multimer polypeptide comprises an amino acid sequence selected from the group consisting of SEQ ID NO: 430 to 435. In some embodiments, the SIRP IgV multimer polypeptide comprises an amino acid sequence selected from the group consisting of SEQ ID NO: 430 to 435, directly linked to the N-terminal of human IgG1 Fc (SEQ ID NO: 135). In some embodiments, a protein configured in FV-6 comprises a SIRP IgV multimer polypeptide comprising three SIRP IgV monomers comprising the same mutation selected from the group consisting of K53H, R69H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a protein configured in FV-6 comprises a SIRP IgV multimer polypeptide comprising three SIRP IgV monomers wherein the three SIRP IgV monomers comprise two different mutation selected from the group consisting of K53H, R69H, Q52H, K68H, Q52H+K68H, K53H+K68H, R69H+K68H, K53H+Q52H and R69H+Q52H, and at most one of the three SIRP IgV monomers comprises a mutation of Q52H, K68H or Q52H+K68H, exhibits higher binding to CD47 at an acidic pH than at physiological pH. In some embodiments, a protein configured in FV-6 comprises a SIRP IgV multimer polypeptide directly linked to the N-terminal of human IgG1 Fc (SEQ ID NO: 135) wherein the SIRP IgV multimer polypeptide comprises an amino acid sequence selected from the group consisting of SEQ ID NO: 430 to 435, exhibits higher binding to CD47 at an acidic pH than at physiological pH, whereas a protein in the same format comprising a SIRP IgV multimer polypeptide sequence selected from the group consisting of SEQ ID NO: 436 to 439, does not exhibit higher binding to CD47 at an acidic pH than at physiological pH.

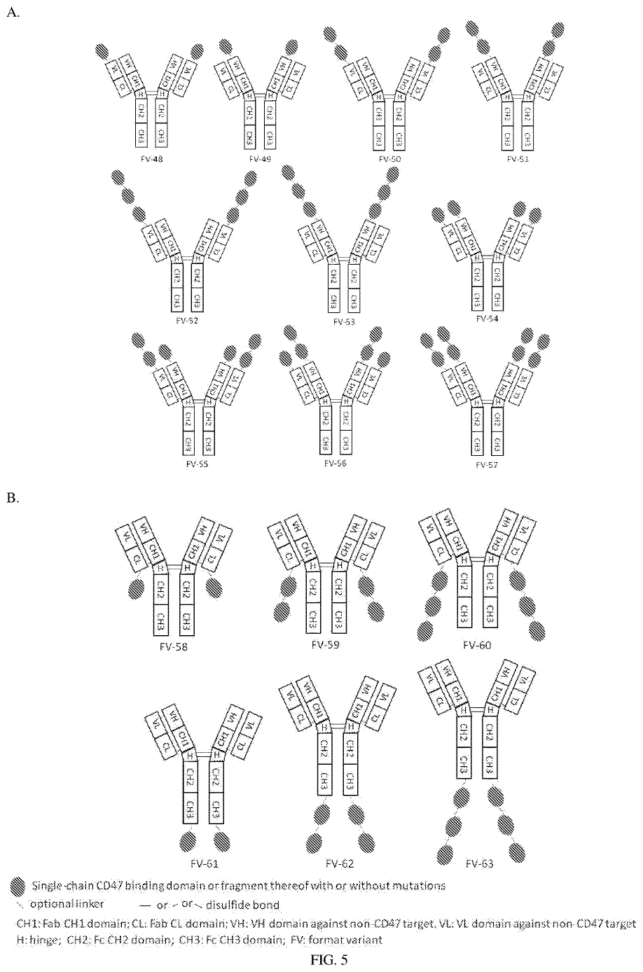

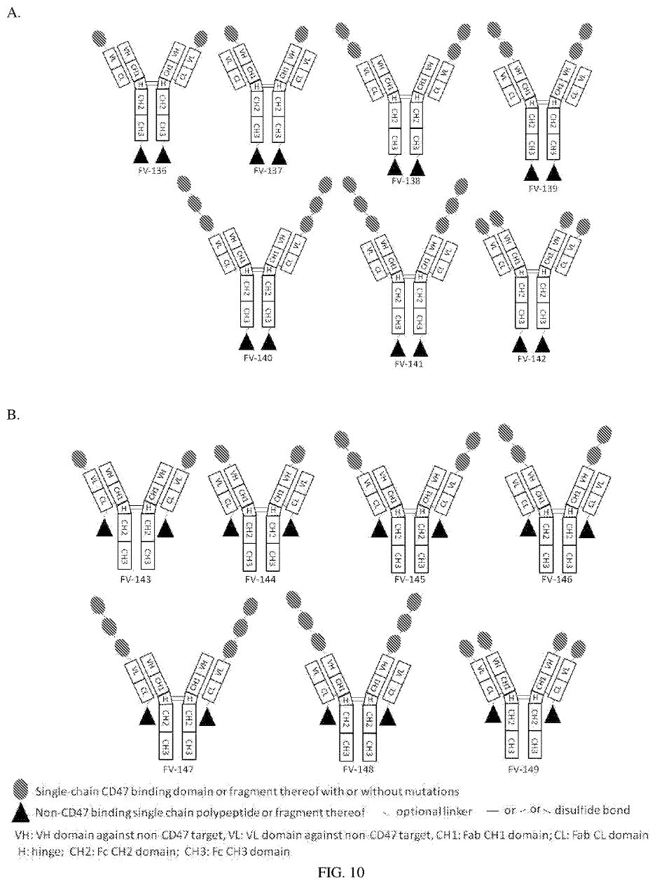

In some embodiments, an antibody fusion protein comprising a said fusion polypeptide comprising a SIRP IgV domain, is configured in a format wherein the antibody fusion protein comprises: 1) a full antibody binding to a non-CD47 antigen comprising two heavy chains and two light chains, wherein the two Fc region of the two heavy chains form a homodimeric Fe, and 2) a SIRP IgV domain, wherein the SIRP IgV domain comprises one, two, three or more SIRP IgV monomers, and is linked preferably through a linker to the N-terminal or C-terminal of the heavy chain or the light chain of the antibody. In certain embodiments, the antibody fusion protein is configured in FV-48 or 49 as set forth in FIG. 5, wherein the SIRP IgV domain comprises one SIRP IgV monomer linked to the N-terminal of the light chain or heavy chain. In certain embodiments, the antibody fusion protein is configured in FV-58 or 61 as set forth in FIG. 5, wherein the SIRP IgV domain comprises one SIRP IgV monomer linked to the C-terminal of the light chain or heavy chain. In certain embodiments, the SIRP IgV monomer is a SIRPα, SIRPβ or SIRPγ IgV monomer. In certain embodiments, the SIRP IgV monomer comprises an amino acid sequence selected from the group consisting of SEQ ID NO: 3 to 81. In some embodiments, the SIRP monomer of the antibody fusion protein configured in FV-48 comprises a mutation of K53H, and the non-CD47 binding Fab domain of the antibody fusion protein comprises an Fab domain or antigen-binding fragment thereof comprising a set of heavy chain variable (VH) and light chain variable (VL) complementarity determining region (CDR) sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-48 comprises a mutation of R69H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-48 comprises a mutation of Q52H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-48 comprises a mutation of K68H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-49 comprises a mutation of K53H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-49 comprises a mutation of R69H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-49 comprises a mutation of Q52H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-49 comprises a mutation of K68H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain of the antibody fusion protein, and the antibody fusion protein comprising the third antigen-binding domain is configured in FV-136 to 137 or FV-143 to 144 as set forth in FIG. 10. In some embodiments, a protein configured in FV-48, FV-49, FV-58, FV-61, FV-143 or FV-144 comprising two SIRP IgV monomers comprising the same mutation selected from the group consisting of K53H, R69H, Q52H, K68H, Q52H+K68H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a protein configured in FV-48 or FV-49 selected from the group of proteins consisting of MP-29, MP-30, MP-31, MP-32, MP-36, MP-37, MP-38, MP-39, MP-40, MP-41, MP-42, MP-46, MP-47 and MP-48 as set forth in Table 20, exhibits higher binding to CD47 at an acidic pH than at physiological pH, whereas a protein configured in FV-48 or FV-49 selected from the group of proteins consisting of MP-3, MP-33, MP-34, MP-35, MP-4, MP-43, MP-44 and MP-45 as set forth in Table 20, does not exhibit higher binding to CD47 at an acidic pH than at physiological pH.

In some embodiments, an antibody fusion protein comprising a said fusion polypeptide comprising a SIRP IgV domain, is configured in a format wherein the antibody fusion protein comprises: 1) a full antibody binding to a non-CD47 antigen comprising two heavy chains and two light chains, wherein the two Fc region of the two heavy chains form a homodimeric Fc, and 2) a SIRP IgV domain, wherein the SIRP IgV domain comprises one, two, three or more SIRP IgV monomers, and is linked preferably through a linker to the N-terminal of the heavy chain or the light chain of the antibody. In certain embodiments, the antibody fusion protein is configured in FV-50 or 51 as set forth in FIG. 5, wherein the SIRP IgV domain comprises a SIRP IgV multimer polypeptide comprising two SIRP IgV monomers. In certain embodiments, the two SIRP IgV monomers of the SIRP IgV multimer polypeptide comprise SIRPα, SIRPβ and/or SIRPγ IgV monomers. In certain embodiments, the two SIRP IgV monomers of the SIRP IgV multimer polypeptide comprise the same or different amino acid sequence and/or mutation. In certain embodiments, the two SIRP IgV monomers of the SIRP IgV multimer polypeptide comprise the same or different amino acid sequence selected from the group consisting of SEQ ID NO: 3 to 81. In certain embodiments, the mutation of the N-terminal monomer and the C-terminal monomer of the SIRP IgV multimer polypeptide comprise a substitution of K53H-K53H, R69H-R69H, K53H-R69H, R69H-K53H, Q52H-R69H, R69H-Q52H, K68H-R69H, R69H-K68H, Q52H-K53H, K53H-Q52H, K68H-K53H, K53H-K68H, Q52H-K68H, K68H-Q52H, Q52H+K68H-R69H, R69H-Q52H+K68H, Q52H+K68H-K53H, or K53H-Q52H+K68H, wherein the “+” indicates concurrent mutation in the same SIRP IgV monomer, while “−” indicates separate mutation of two neighboring SIRP IgV monomers serially linked from the N-terminal to the C-terminal wherein the mutation from the left to the right of the “−” indicates the mutation from the N-terminal monomer to the C-terminal monomer. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain of the antibody fusion protein, and the antibody fusion protein comprising the third antigen-binding domain is configured in FV-138 to 139 or FV-145 to 146 as set forth in FIG. 10. In some embodiments, the non-CD47 binding Fab domain of the antibody fusion protein comprises an Fab domain or antigen-binding fragment thereof comprising a set of heavy chain variable (VH) and light chain variable (VL) complementarity determining region (CDR) sequences at least 30%, at least 40%, at least 50%, at least 60%, at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, a protein configured in FV-50, FV-51, FV-138, FV-139, FV-145 or FV-146 comprises a SIRP IgV multimer polypeptide comprising two SIRP IgV monomers comprising the same mutation selected from the group consisting of K53H, R69H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a protein configured in FV-50, FV-51, FV-138, FV-139, FV-145 or FV-146 comprises a SIRP IgV multimer polypeptide comprising two SIRP IgV monomers wherein the two SIRP IgV monomers comprise two different mutation selected from the group consisting of K53H, R69H, Q52H, K68H, Q52H+K68H, K53H+K68H, R69H+K68H, K53H+Q52H and R69H+Q52H, and at most one of the two SIRP IgV monomers comprises a mutation of Q52H, K68H or Q52H+K68H, exhibits higher binding to CD47 at an acidic pH than at physiological pH. In some embodiments, a protein configured in FV-50 or FV-51 selected from the group of proteins consisting of MP-12, MP-13, MP-14, MP-15, MP-54, MP-58, MP-20, MP-21, MP-59, MP-60 and MP-61 as set forth in Table-20, exhibits higher binding to CD47 at an acidic pH than at physiological pH, whereas a protein configured in FV-50 or FV-51 selected from the group of proteins consisting of MP-55, MP-56, MP-57, MP-62 and MP-63 does not exhibit higher binding to CD47 at an acidic pH than at physiological pH.

In some embodiments, an antibody fusion protein comprising a said fusion polypeptide comprising a SIRP IgV domain is configured in a format, wherein the antibody fusion protein comprises: 1) a full antibody binding to a non-CD47 antigen comprising two heavy chains and two light chains, wherein the two Fc region of the two heavy chains form a homodimeric Fe, and 2) a SIRP IgV domain, wherein the SIRP IgV domain comprises one, two, three or more SIRP IgV monomers, and is linked preferably through a linker to the N-terminal of the heavy chain or the light chain of the antibody. In certain embodiments, the antibody fusion protein is configured in FV-52 or 53 as set forth in FIG. 5, wherein the SIRP IgV domain comprises a SIRP IgV multimer polypeptide comprising three SIRP IgV monomers. In certain embodiments, the three SIRP IgV monomers of the SIRP IgV multimer polypeptide comprise SIRPα, SIRPβ and/or SIRPγ IgV monomers. In certain embodiments, the three SIRP IgV monomers of the SIRP IgV multimer polypeptide comprise the same or different amino acid sequence and/or mutation. In certain embodiments, the three SIRP IgV monomers of the SIRP IgV multimer polypeptide comprise the same or different amino acid sequence selected from the group consisting of SEQ ID NO: 3 to 81. In certain embodiments, the three SIRP IgV monomers of the SIRP IgV multimer polypeptide from the N-terminal monomer to the middle monomer to the C-terminal monomer comprise of K53H-K53H-K53H, R69H-R69H-R69H, K53H-K53H-R69H, R69H-K53H-K53H, a substitution K53H-R69H-K53H, K53H-R69H-R69H, R69H-R69H-K53H, R69H-K53H-R69H, K53H-K53H-Q52H, Q52H-K53H-K53H, K53H-Q52H-K53H, K53H-K53H-K68H, K68H-K53H-K53H, K53H-K68H-K53H, Q52H-R69H-R69H, R69H-R69H-Q52H, R69H-Q52H-R69H, K68H-R69H-R69H, R69H-R69H-K68H, or R69H-K68H-R69H, wherein the “−” indicates separate mutation of two neighboring SIRP IgV monomers serially linked from the N-terminal to the C-terminal wherein the mutation from the left to the right of the “−” indicates the mutation from the N-terminal monomer to the C-terminal monomer. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain of the antibody fusion protein, and the antibody fusion protein comprising the third antigen-binding domain is configured in FV-140 to 141 or FV-147 to 148 as set forth in FIG. 10.

In some embodiments, an antibody fusion protein comprising a said fusion polypeptide comprising a SIRP IgV domain is configured in a format, wherein the antibody fusion protein comprises: 1) a full antibody binding to a non-CD47 antigen comprising two heavy chains and two light chains, wherein the two Fc region of the two heavy chains form a homodimeric Fc, and 2) a SIRP IgV domain, wherein the SIRP IgV domain comprises one, two, three or more SIRP IgV monomers, and is linked preferably through a linker to the C-terminal of the heavy chain and/or the light chain of the antibody. In certain embodiments, the antibody fusion protein is configured in FV-58 to 63 as set forth in FIG. 5. In certain embodiments, the SIRP IgV monomers of the SIRP IgV domain comprise SIRPα, SIRPβ and/or SIRPγ IgV monomers. In certain embodiments, the three SIRP IgV monomers of the SIRP IgV domain comprise the same or different amino acid sequence and/or mutation. In certain embodiments, the three SIRP IgV monomers of the SIRP IgV domain comprise the same or different amino acid sequence selected from the group consisting of SEQ ID NO: 3 to 81.

In some embodiments, an antibody fusion protein comprising a said fusion polypeptide comprising a SIRP IgV domain is configured in a format wherein the antibody fusion protein comprises: 1) a full antibody binding to a non-CD47 antigen comprising two heavy chains and two light chains, wherein the two Fc region of the two heavy chains form a homodimeric Fe, and 2) a SIRP IgV domain, wherein the SIRP IgV domain comprises one, two, three or more SIRP IgV monomers, and is linked preferably through a linker to the N-terminal of the heavy chain and the light chain of the antibody. In certain embodiments, the antibody fusion protein is configured in FV-54 as set forth in FIG. 5, wherein the SIRP IgV domain comprises one SIRP IgV monomer. In certain embodiments, the four SIRP IgV monomers of the antibody fusion protein comprise SIRPα, SIRPβ and/or SIRPγ IgV monomers. In certain embodiments, the two SIRP IgV monomers linked to the N-terminal of the antibody light chain and heavy chain comprise the same or different amino acid sequence and/or mutation. In certain embodiments, the two SIRP IgV monomers linked to the N-terminal of the antibody light chain and heavy chain comprise the same or different amino acid sequence selected from the group consisting of SEQ ID NO: 3 to 81. In certain embodiments, the two SIRP IgV monomers comprise mutation of K53HxK53H, R69HxR69H, K53HxR69H, K53HxQ52H, K53HxK68H, Q52HxR69H, or K68HxR69H, wherein the “x” indicates separate mutation in the two separate SIRP IgV domains linked to the N-terminal of the heavy chain and light chain from the left to the right of the “x” symbol or the N-terminal of the light chain and heavy chain from the left to the right of the “x” symbol. In some embodiments, a protein configured in FV-54 wherein the SIRP IgV monomers linked to the N-terminal of the heavy chain and light chain comprise the same mutation selected from the group consisting of K53H, R69H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a protein configured in FV-54 wherein the SIRP IgV monomers linked to the N-terminal of the heavy chain and light chain comprise two different mutations selected from the group consisting of K53H, R69H, K53H+K68H, R69H+K68H, K53H+Q52H and R69H+Q52H, exhibits higher binding to CD47 at an acidic pH than at physiological pH. In some embodiments, a protein configured in FV-54 selected from the group of proteins consisting of MP-6, MP-7, MP-8, MP-9, MP-10, MP-11, MP-16, MP-49 and MP-50 as set forth in Table 20, exhibits higher binding to CD47 at an acidic pH than at physiological pH, whereas a protein configured in FV-54 selected from the group of proteins consisting of MP-5, MP-51, MP-52 and MP-53, does not exhibit higher binding to CD47 at an acidic pH than at physiological pH. In some embodiments, the antibody fusion protein comprises an additional SIRP IgV monomer linked preferably through a linker to the N-terminal of one or both of the SIRP IgV monomers linked to the N-terminal of the heavy chain and light chain, wherein the new antibody fusion protein is configured in a format of FV-55, 56 or 57. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain of the antibody fusion protein, and the antibody fusion protein comprising the third antigen-binding domain is configured in FV-142 or FV-149 as set forth in FIG. 10.

In some embodiments, the homodimeric Fc of the Fe fusion protein or antibody fusion protein, as for example configured in FV-2 to 14, 48 to 63, and 136 to 149 above, comprises a human IgG1, human IgG4 or human IgG2 Fc, with wild type, reduced, or abolished effector function. In certain embodiments, the homodimeric Fc comprises an amino acid sequence selected from the group consisting of SEQ ID NO: 130 to 135. In certain embodiments, the homodimeric Fc comprises a human IgG1 Fc with wild type effector function comprising an amino acid sequence of SEQ ID NO: 135. In certain embodiments, the homodimeric Fe comprises a human IgG1 Fc with enhanced effector function, comprising mutations known in the arts to enhance the effector function of human IgG1 Fc. In certain embodiments, the Fc region is a human IgG1 Fe with enhanced antibody-dependent cellular phagocytosis (ADCP) function, comprising a mutation selected from the group consisting of G236A, S239D/1332E, G236A/S239D/1332E, S239D/A330L/1332E, G236A/A330L/1332E, G236A/S239D/A330L/1332E, and F243L/R292P/Y300L/V305I/P396L, according to the EU numbering scheme.

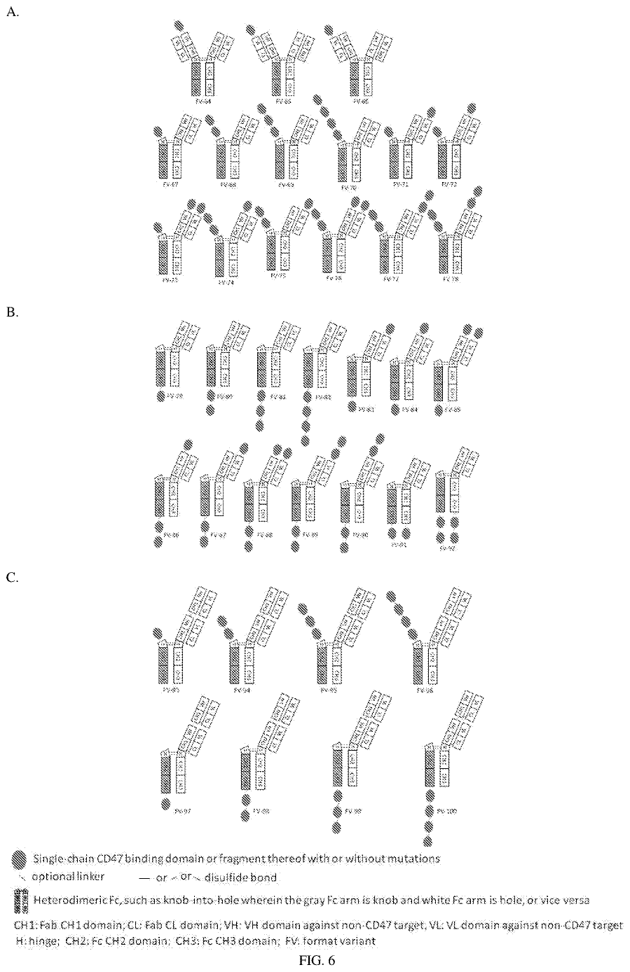

In some embodiments, an antibody fusion protein comprising a said fusion polypeptide comprising a SIRP IgV domain, is configured in a format wherein the antibody fusion protein comprises: 1) a first heavy chain and a second heavy chain each comprising VH-CH1-binge-CH2-CH3, wherein the two heavy chains form a heterodimeric Fc; 2) a light chain comprising VL-CL, wherein the light chain pairs with the two heavy chains to form two Fab domains binding to a non-CD47 antigen; and 3) a SIRP IgV monomer, wherein the SIRP IgV monomer is linked preferably through a linker to the N-terminal of one of the two heterodimeric heavy chains. In certain embodiments, the antibody fusion protein is configured in FV-64 as set forth in FIG. 6. In certain embodiments, the SIRP IgV monomer is a SIRPα, SIRPβ or SIRPγ IgV monomer. In certain embodiments, the SIRP IgV monomer comprises an amino acid sequence selected from the group consisting of SEQ ID NO: 3 to 81.

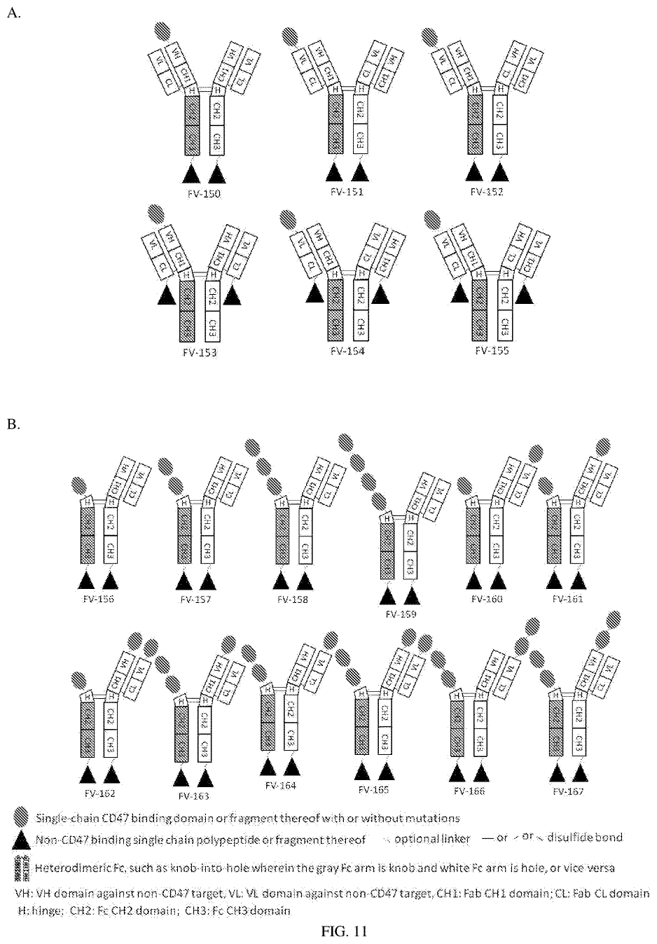

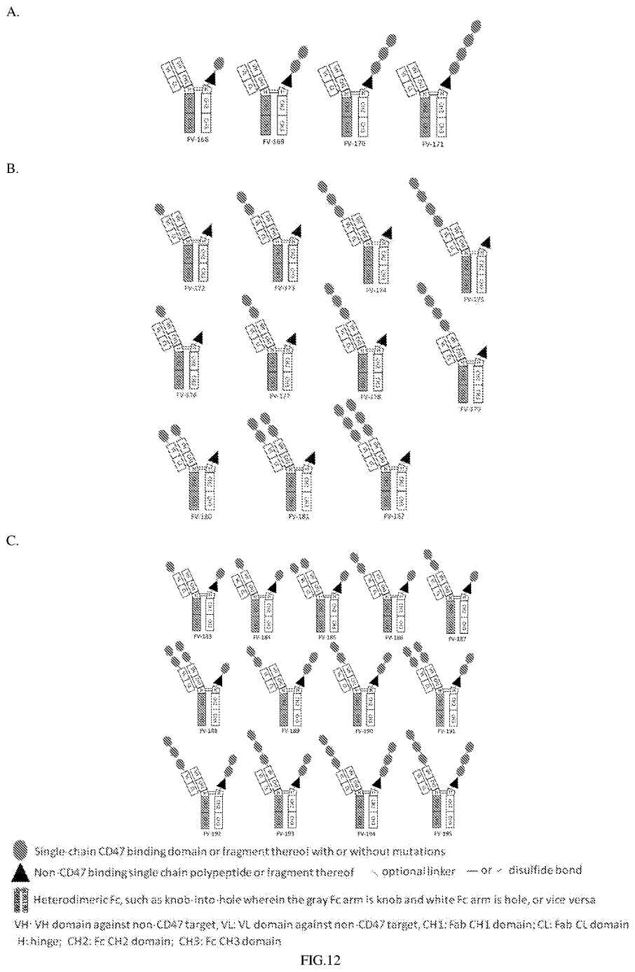

In certain embodiments, the SIRP IgV monomer comprises a SIRPβ or SIRPγ IgV monomer. In certain embodiments, the SIRP IgV monomer comprises a SIRPβ or SIRPγ IgV monomer, comprising an amino acid sequence selected from the group consisting of SEQ ID NO: 12 to 21, 31 to 33, 43 to 45, 55 to 57, 67 to 69, and 79 to 81. In certain embodiments, the non-CD47 binding Fab domain of the antibody fusion protein binds to an antigen associated with a diseased cell or tissue. In some embodiments, the SIRP monomer of the antibody fusion protein configured in FV-64 comprises a SEQ ID NO: 3, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-64 comprises a SEQ ID NO: 4, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-64 comprises a SEQ ID NO: 10, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-64 comprises a mutation of K53H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-64 comprises a mutation of R69H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-64 comprises a mutation of Q52H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-64 comprises a mutation of K68H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain of the antibody fusion protein, and the antibody fusion protein comprising the third antigen-binding domain is configured in FV-150 or FV-153 as set forth in FIG. 11. In certain embodiments, the antibody fusion protein configured in FV-150 or 153, wherein the Fab domain binds to an antigen associated with a diseased cell or tissue and the single-chain third antigen-binding domain binds to a surface receptor of an effector cell. In some embodiments, a protein configured in FV-64, FV-150 or FV-153, comprises one SIRP IgV monomer comprising a mutation selected from the group consisting of K53H, R69H, Q52H, K68H and Q52H+K68H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a protein configured in FV-64 selected from the group of proteins consisting of MP-75, MP-76, MP-77, MP-79 and MP-80 as set forth in Table 20, exhibits higher binding to CD47 at an acidic pH than at physiological pH, whereas a protein configured in FV-64 selected from the group of proteins consisting of MP-74 and MP-78 does not exhibit higher binding to CD47 at an acidic pH than at physiological pH.

In some embodiments, an antibody fusion protein comprising a said fusion polypeptide comprising a SIRP IgV domain, is configured in a format wherein the antibody fusion protein comprises: 1) a first heavy chain comprising VH-CH1-hinge-CH2-CH3 and a second chimeric heavy chain comprising VL-CL-binge-CH2-CH3 from the N-terminal to C-terminal, wherein the two heavy chains form a heterodimeric Fc; 2) a light chain comprising VL-CL, wherein the light chain pairs with the first heavy chains to form a Fab domain binding to a non-CD47 antigen; 3) a Fd chain comprising VH-CH1, wherein the Fd chain pairs with VL-CL part of the second chimeric heavy chain to form a Fab domain binding to a non-CD47 antigen; and 4) a SIRP IgV monomer, wherein the SIRP IgV monomer is linked preferably through a linker to the N-terminal of the light chain, the Fd chain, the first heavy chain, or the chimeric heavy chain. In certain embodiments, the antibody fusion protein is configured in FV-65 as set forth in FIG. 6, wherein the SIRP IgV monomer is linked to the N-terminal of the light chain. In certain embodiments, the SIRP IgV monomer is a SIRPα, SIRPβ or SIRPγ IgV monomer. In certain embodiments, the SIRP IgV monomer comprises an amino acid sequence selected from the group consisting of SEQ ID NO: 3 to 81. In certain embodiments, the SIRP IgV monomer comprises a SIRPβ or SIRPγ IgV monomer. In certain embodiments, the SIRP IgV monomer comprises a SIRPβ or SIRPγ IgV monomer, comprising an amino acid sequence selected from the group consisting of SEQ ID NO: 12 to 21, 31 to 33, 43 to 45, 55 to 57, 67 to 69, and 79 to 81. In some embodiments, the SIRP monomer of the antibody fusion protein configured in FV-65 comprises a SEQ ID NO: 3, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-65 comprises a SEQ ID NO: 4, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-65 comprises a SEQ ID NO: 10, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-65 comprises a mutation of K53H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-65 comprises a mutation of Q52H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-65 comprises a mutation of K68H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In certain embodiments, the Fab domain binds to an antigen associated with a diseased cell or tissue. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain of the antibody fusion protein, and the antibody fusion protein comprising the third antigen-binding domain is configured in FV-151 or FV-154 as set forth in FIG. 11. In certain embodiments, the antibody fusion protein configured in FV-151 or 154, wherein the Fab domain binds to an antigen associated with a diseased cell or tissue and the single-chain third antigen-binding domain binds to a surface receptor of an effector cell.

In some embodiments, an antibody fusion protein comprising a said fusion polypeptide comprising a SIRP IgV domain, is configured in a format wherein the antibody fusion protein comprises: 1) a first heavy chain comprising VH-CH1-hinge-CH2-CH3 and a second chimeric heavy chain comprising VH-CL-hinge-CH2-CH3 from the N-terminal to C-terminal, wherein the two heavy chains form a heterodimeric Fe; 2) a first light chain comprising VL-CL, wherein the light chain pairs with the first heavy chains to form a Fab domain binding to a non-CD47 antigen; 3) a second chimeric light chain comprising VL-CH1, wherein the chimeric light chain pairs with VH-CL part of the second chimeric heavy chain to form a Fab domain binding to a non-CD47 antigen; and 4) a SIRP IgV monomer, wherein the SIRP IgV monomer is linked preferably through a linker to the N-terminal of the first light chain, the chimeric light chain, the first heavy chain, or the chimeric heavy chain. In certain embodiments, the antibody fusion protein is configured in FV-66 as set forth in FIG. 6, wherein the SIRP IgV monomer is linked to the N-terminal of the first light chain. In certain embodiments, the SIRP IgV monomer is a SIRPα, SIRPβ or SIRPγ IgV monomer. In certain embodiments, the SIRP IgV monomer comprises an amino acid sequence selected from the group consisting of SEQ ID NO: 3 to 81. In certain embodiments, the SIRP IgV monomer comprises a SIRPβ or SIRPγ IgV monomer. In certain embodiments, the SIRP IgV monomer comprises a SIRPβ or SIRPγ IgV monomer, comprising an amino acid sequence selected from the group consisting of SEQ ID NO: 12 to 21, 31 to 33, 43 to 45, 55 to 57, 67 to 69, and 79 to 81. In some embodiments, the SIRP monomer of the antibody fusion protein configured in FV-66 comprises a SEQ ID NO: 3, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-66 comprises a SEQ ID NO: 4, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-66 comprises a SEQ ID NO: 10, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-66 comprises a mutation of K53H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-66 comprises a mutation of Q52H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-66 comprises a mutation of K68H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In certain embodiments, the Fab domain binds to an antigen associated with a diseased cell or tissue. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain of the antibody fusion protein, and the antibody fusion protein comprising the third antigen-binding domain is configured in FV-152 or FV-155 as set forth in FIG. 11. In certain embodiments, the antibody fusion protein configured in FV-152 or 155, wherein the Fab domain binds to an antigen associated with a diseased cell or tissue and the single-chain third antigen-binding domain binds to a surface receptor of an effector cell.

In some embodiments, an antibody fusion protein comprising a said fusion polypeptide comprising a SIRP IgV domain is configured in a format wherein the antibody fusion protein comprises: 1) a first polypeptide comprising a SIRP IgV domain and a Fc region, wherein the SIRP IgV domain comprises one, two, three, four or more of SIRP IgV monomers, and is linked to the N-terminal of the Fc region, 2) a full heavy chain of an antibody and a full light chain of an antibody, wherein the antibody heavy chain and the light chain pair to form an half-antibody that binds to a non-CD47 antigen, and 3) the Fe region of the first polypeptide and the antibody heavy chain form a heterodimeric Fc. In certain embodiments, the antibody fusion protein is configured in FV-67 as set forth in FIG. 6, wherein the SIRP IgV domain comprises one SIRP IgV monomer. In certain embodiments, the SIRP IgV monomer is a SIRPα, SIRPβ or SIRPγ IgV monomer. In certain embodiments, the SIRP IgV monomer comprises an amino acid sequence selected from the group consisting of SEQ ID NO: 3 to 81. In certain embodiments, the SIRP IgV monomer comprises a SIRPβ or SIRPγ IgV monomer. In certain embodiments, the SIRP IgV monomer comprises a SIRPβ or SIRPγ IgV monomer, comprising an amino acid sequence selected from the group consisting of SEQ ID NO: 12 to 21, 31 to 33, 43 to 45, 55 to 57, 67 to 69, and 79 to 81. In some embodiments, the SIRP monomer of the antibody fusion protein configured in FV-67 comprises a mutation of K53H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-67 comprises a mutation of R69H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-67 comprises a mutation of Q52H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-67 comprises a mutation of K68H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain or the SIRP IgV domain of the antibody fusion protein. In certain embodiments, the antibody fusion protein comprising the third antigen-binding domain is configured in FV-156 or FV-168 as set forth in FIG. 11 or 12 respectively. In certain embodiments, the antibody fusion protein is configured in 156 or FV-168, wherein the Fab domain binds to an antigen associated with a diseased cell or tissue and the single-chain third antigen-binding domain binds to a surface receptor of an effector cell, or vice versa. In some embodiments, a protein configured in FV-67, FV-156 or FV-168, comprises one SIRP IgV monomer comprising a mutation selected from the group consisting of K53H, R69H, Q52H, K68H and Q52H+K68H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a protein configured in FV-67 selected from the group of proteins consisting of MP-25, MP-26, MP-67, MP-68, MP-71, MP-72 and MP-73 as set forth in Table 20, exhibits higher binding to CD47 at an acidic pH than at physiological pH, whereas a protein configured in FV-67 selected from the group of proteins consisting of MP-24, MP-69 and MP-70 does not exhibit higher binding to CD47 at an acidic pH than at physiological pH.

In some embodiments, an antibody fusion protein comprising a said fusion polypeptide comprising a SIRP IgV domain is configured in a format wherein the antibody fusion protein comprises: 1) a first polypeptide comprising a SIRP IgV domain and a Fc region, wherein the SIRP IgV domain comprises one, two, three, four or more of SIRP IgV monomers, and is linked to the N-terminal of the Fe region, 2) a full heavy chain of an antibody and a full light chain of an antibody, wherein the antibody heavy chain and the light chain pair to form an half-antibody that binds to a non-CD47 antigen, and 3) the Fe region of the first polypeptide and the antibody heavy chain form a heterodimeric Fc. In certain embodiments, the antibody fusion protein is configured in FV-68, 69 or 70 as set forth in FIG. 6, wherein the SIRP IgV domain comprises a SIRP IgV multimer polypeptide comprising two, three or four SIRP IgV monomers. In certain embodiments, the two, three or four SIRP IgV monomers of the SIRP IgV multimer polypeptide comprise SIRPα, SIRPβ and/or SIRPγ IgV monomers. In certain embodiments, the two, three or four SIRP IgV monomers of the SIRP IgV multimer polypeptide comprise the same or different amino acid sequence and/or mutation. In certain embodiments, the two, three or four SIRP IgV monomers of the SIRP IgV multimer polypeptide comprise the same or different amino acid sequence selected from the group consisting of SEQ ID NO: 3 to 81. In certain embodiments, the two, three of four SIRP IgV monomers of the SIRP IgV multimer polypeptide from the N-terminal monomer to the C-terminal monomer comprise substitution as described for such SIRP IgV multimer polypeptides earlier in present disclosure. In certain embodiments, the SIRP IgV multimer polypeptide comprises an amino acid sequence selected from the group consisting of SEQ ID NO: 82 to 113, SEQ ID NO: 418 to 425 and SEQ ID NO: 430 to 435. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain or the SIRP IgV domain of the antibody fusion protein. In certain embodiments, the antibody fusion protein comprising the third antigen-binding domain is configured in FV-157 to 159 or FV-169 to 171 as set forth in FIGS. 11 and 12, respectively. In certain embodiments, the antibody fusion protein is configured in FV-157 to 159 or FV-169 to 171, wherein the Fab domain binds to an antigen associated with a diseased cell or tissue and the single-chain third antigen-binding domain binds to a surface receptor of an effector cell, or vice versa. In some embodiments, the non-CD47 binding Fab domain of the antibody fusion protein comprises an Fab domain or antigen-binding fragment thereof comprising a set of heavy chain variable (VH) and light chain variable (VL) complementarity determining region (CDR) sequences at least 30%, at least 40%, at least 50%, at least 60%, at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4.

In some embodiments, an antibody fusion protein comprising a said fusion polypeptide comprising a SIRP IgV domain is configured in a format wherein the antibody fusion protein comprises: 1) a first polypeptide comprising a first SIRP IgV domain and a Fe region, wherein the first SIRP IgV domain comprises one, two, three, four or more of SIRP IgV monomers, and is linked to the N-terminal of the Fc region, 2) a full heavy chain of an antibody and a full light chain of an antibody, wherein the antibody heavy chain and the light chain pair to form an half-antibody that binds to a non-CD47 antigen, 3) the Fc region of the first polypeptide and the antibody heavy chain form a heterodimeric Fc, and 4) a second SIRP IgV domain, wherein the second SIRP IgV domain comprises one, two, three, four or more of SIRP IgV monomers and is linked preferably through a linker to the N-terminal of light chain and/or heavy chain. In certain embodiments, the antibody fusion protein is configured in FV-71 or 72 as set forth in FIG. 6, wherein the first SIRP IgV domain comprises one SIRP IgV monomer and the second SIRP IgV domain comprises one SIRP IgV monomer linked to the N-terminal of the light chain or heavy chain. In certain embodiments, the antibody fusion protein is configured in FV-73 as set forth in FIG. 6, wherein the first SIRP IgV domain comprises one SIRP IgV monomer and the second SIRP IgV domain comprises one SIRP IgV monomer linked to the N-terminal of the light chain and heavy chain. In certain embodiments, the antibody fusion protein is configured in FV-74, 75 or 76 as set forth in FIG. 6, wherein the first SIRP IgV domain comprises a SIRP IgV multimer polypeptide of present disclosure comprising two SIRP IgV monomers and the second SIRP IgV domain comprises one SIRP IgV monomer linked to the N-terminal of the light chain and/or heavy chain. In certain embodiments, the antibody fusion protein is configured in FV-77 or 78 as set forth in FIG. 6, wherein the first SIRP IgV domain comprises a first SIRP IgV multimer polypeptide of present disclosure comprising two SIRP IgV monomers, and the second SIRP IgV domain comprises a second SIRP IgV multimer polypeptide of present disclosure comprising two SIRP IgV monomers and is linked to the N-terminal of the light chain or heavy chain. In certain embodiments, the SIRP IgV monomers of the antibody fusion protein configured in FV-71 to 78 comprise SIRPα, SIRPβ and/or SIRPγ IgV monomers. In certain embodiments, the SIRP IgV monomers comprise the same or different amino acid sequence and/or mutation. In certain embodiments, the SIRP IgV monomers comprise the same or different amino acid sequences selected from the group consisting of SEQ ID NO: 3 to 81. In certain embodiments, the first SIRP IgV domain linked to the N-terminal of the Fc chain comprises SIRPβ and/or SIRPγ IgV monomer(s). In certain embodiments, the first SIRP IgV domain linked to the N-terminal of the Fc chain comprises SIRPβ and/or SIRPγ IgV monomer(s) comprising the same or different amino acid sequence selected from the group consisting of SEQ ID NO: 12 to 21, 31 to 33, 43 to 45, 55 to 57, 67 to 69, and 79 to 81. In certain embodiments, the first SIRP IgV domain linked to the N-terminal of the Fc chain comprises SIRPα IgV monomer(s) comprising the same or different amino acid sequence selected from the group consisting of SEQ ID NO: 3 to 11, 22 to 30, 34 to 42, 46 to 54, 58 to 66, and 70 to 78. In certain embodiments, the first SIRP IgV domain linked to the N-terminal of the Fc chain comprises SIRPβ and/or SIRPγ IgV monomer(s), and the second SIRP IgV domain comprises SIRPα, SIRPβ and/or SIRPγ IgV monomer(s). In certain embodiments, the first SIRP IgV domain linked to the N-terminal of the Fc chain comprises SIRPβ IgV monomer(s), and the second SIRP IgV domain comprises SIRPα, SIRPβ and/or SIRPγ IgV monomer(s). In certain embodiments, the first SIRP IgV domain linked to the N-terminal of the Fc chain comprises SIRPγ IgV monomer(s), and the second SIRP IgV domain comprises SIRPα, SIRPβ and/or SIRPγ IgV monomer(s). In certain embodiments, the SIRP IgV multimer polypeptides of the first SIRP IgV domain and/or the second SIRP IgV domain comprise the same or different amino acid sequence selected from the group consisting of SEQ ID NO: 82 to 96 and SEQ ID NO: 418 to 425. In certain embodiments, the antibody fusion protein configured in FV-71 or 72, wherein the first SIRP IgV domain linked to the N-terminal of the Fc chain comprises one SIRPα IgV monomer and the second SIRP IgV domain linked to the N-terminal of the heavy chain or light chain comprises one SIRPα, SIRPβ or SIRPγ IgV monomer. In certain embodiments, the antibody fusion protein configured in FV-71 or 72, wherein the first SIRP IgV domain linked to the N-terminal of the Fc chain comprises one SIRPα IgV monomer comprising an amino acid sequence selected from the group consisting of SEQ ID NO: 3 to 11, and the second SIRP IgV domain linked to the N-terminal of the heavy chain or light chain comprises one SIRPα, SIRPβ or SIRPγ IgV monomer comprising a mutation selected from the group consisting of K53H+K68H, R69H+K68H, K53H+Q52H, R69H+Q52H, R69H+Q37H, K53H+Q37H, R69H, K53H, K96H, V33H, and P35H. In some embodiments, the SIRP monomer of the antibody fusion protein configured in FV-71 comprises a mutation of K53H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-71 comprises a mutation of R69H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-71 comprises a mutation of Q52H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-71 comprises a mutation of K68H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-72 comprises a mutation of K53H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-72 comprises a mutation of R69H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-72 comprises a mutation of Q52H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the antibody fusion protein configured in FV-72 comprises a mutation of K68H, and the non-CD47 binding Fab domain of the antibody fusion protein comprises an Fab domain or antigen-binding fragment thereof comprising a set of VH and VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the antibody fusion protein comprises additionally a third antigen-binding domain, wherein the third antigen-binding domain is a single-chain polypeptide binding to a third antigen and is linked through a linker to the C-terminal of the heavy chain or light chain or the SIRP IgV domain of the antibody fusion protein. In certain embodiments, the antibody fusion protein comprising the third antigen-binding domain is configured in FV-160 to 167 or FV-183 to 190 as set forth in FIGS. 11 and 12, respectively. In certain embodiments, the antibody fusion protein is configured in FV-160 to 167 or FV-183 to 190, wherein the Fab domain binds to an antigen associated with a diseased cell or tissue and the single-chain third antigen-binding domain binds to a surface receptor of an effector cell, or vice versa. In some embodiments, a protein configured in FV-71, FV-72, FV-160 or FV-161, comprises two SIRP IgV monomers comprising the same mutation selected from the group consisting of K53H, R69H, Q52H, K68H, Q52H+K68H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a protein configured in FV-71, FV-72, FV-160 or FV-161 comprises two SIRP IgV monomers comprising two different mutations selected from the group consisting of K53H, R69H, Q52H, K68H, Q52H+K68H, K53H+K68H, R69H+K68H, K53H+Q52H and R69H+Q52H, exhibits higher binding to CD47 at an acidic pH than at physiological pH. In some embodiments, a protein configured in FV-72 selected from the group of proteins consisting of MP-27, MP-28, MP-81 and MP-82 as set forth in Table 20, exhibits higher binding to CD47 at an acidic pH than at physiological pH, whereas a protein configured in FV-72 selected from the group of proteins consisting of MP-83 and MP-84 does not exhibit higher binding to CD47 at an acidic pH than at physiological pH.

In some embodiments, an antibody fusion protein comprising a said fusion polypeptide comprising a SIRP IgV domain, is configured in FV-128 as set forth in FIG. 9, wherein the antibody fusion protein comprises: 1) a first polypeptide comprising a SIRP IgV monomer and a Fe region, wherein the SIRP IgV monomer is directly linked to the N-terminal of the Fc region; 2) a chimeric heavy chain comprising from N-terminal to C-terminal VH2-CL-linker-VH1-CH1-Hinge-CH2-CH3, wherein the chimeric heavy chain forms a heterodimeric Fc with the Fe region of the first polypeptide; 3) a first light chain comprising VL1-CL, wherein the light chain pairs with the VH1-CH1 part of the chimeric heavy chain to form the first Fab domain; 4) a second chimeric light chain comprising VL2-CH1, wherein the chimeric light chain pairs with the VH2-CL part of the chimeric heavy chain to form the 2^{nd} Fab domain; and 5) the first and second Fab domains bind to two different non-CD47 antigens. In certain embodiments, the SIRP IgV monomer is a SIRPα, SIRPβ or SIRPγ IgV monomer. In certain embodiments, the SIRP IgV monomer comprises an amino acid sequence selected from the group consisting of SEQ ID NO: 3 to 81. In certain embodiments, the SIRP IgV monomer comprises a SIRPβ or SIRPγ IgV monomer. In certain embodiments, the SIRP IgV monomer comprises a SIRPβ or SIRPγ IgV monomer, comprising an amino acid sequence selected from the group consisting of SEQ ID NO: 12 to 21, 31 to 33, 43 to 45, 55 to 57, 67 to 69, and 79 to 81. In certain embodiments, the linker of the chimeric heavy chain comprises an amino acid sequence of GGGGSGGGGS. In certain embodiments, the first Fab domain binds to a surface receptor of an effector cell and the second Fab domain binds to an antigen associated with a diseased cell or tissue, or vice versa. In certain embodiment, the first Fab domain binds to a surface receptor of an effector cell selected from the group of effector cell surface receptors included in Table 4 and the second Fab domain binds to a disease associated antigen selected from the group of disease associated antigens included in Table 2 and 3, or vice versa. In certain embodiment, the first Fab domain is derived from an effector cell surface receptor-binding antibody included in Table 4 and the second Fab domain is derived from a disease associated antigen-binding antibody included in Table 2 and 3, or vice versa. In certain embodiment, the first Fab domain binds to a cell adhesion molecule and the second Fab domain binds to an antigen associated with a diseased cell or tissue, or vice versa. In certain embodiment, the first Fab domain binds to a cell adhesion molecule selected from the group of cell adhesion molecules included in Table 3 and the second Fab domain binds to a disease associated antigen selected from the group of disease associated antigens included in Table 2, or vice versa. In certain embodiment, the first Fab domain is derived from a cell adhesion molecule-binding antibody included in Table 3 and the second Fab domain is derived from a disease associated antigen-binding antibody included in Table 2, or vice versa. In certain embodiment, the first Fab domain binds to an antigen selected from the group consisting of CDH3, Nectin-4, EpCAM, ICAM-1, CEACAM5, and CD44, and the second Fab domain binds to an antigen selected from the group consisting of HER2, EGFR, VEGFR2, VEGFR1, VEGF, FGFR2, GD2, B7-H3, PD-L1, ROR1, CLDN18.2, CLDN6, CD70, CD117, CD38, SLAM7, BCMA, GPRC5D, FcRH5, CD30, CD20, CD19, CD22, CD37 and CD52, or vice versa.

In some embodiments, an antibody fusion protein comprising a said fusion polypeptide comprising a SIRP IgV domain, is configured in FV-129, 130 or I31 as set forth in FIG. 9, wherein the antibody fusion protein comprises: 1) a first polypeptide comprising a Fc region and a SIRP IgV multimer polypeptide of present disclosure comprising two, three or four SIRP IgV monomers, wherein the SIRP IgV multimer polypeptide is directly linked to the N-terminal of the Fc region; 2) a chimeric heavy chain comprising from N-terminal to C-terminal VH2-CL-linker-VH1-CH1-Hinge-CH2-CH3, wherein the chimeric heavy chain forms a heterodimeric Fc with the Fc region of the first polypeptide; 3) a first light chain comprising VL1-CL, wherein the light chain pairs with the VH1-CH1 part of the chimeric heavy chain to form the first Fab domain; 4) a second chimeric light chain comprising VL2-CH1, wherein the chimeric light chain pairs with the VH2-CL part of the chimeric heavy chain to form the 2^{nd} Fab domain; and 5) the first and second Fab domains bind to two different non-CD47 antigens. In certain embodiments, the SIRP IgV monomers of the SIRP IgV multimer polyeptide comprise SIRPα, SIRPβ and/or SIRPγ IgV monomers. In certain embodiments, the SIRP IgV monomers of the SIRP IgV multimer polyeptide comprise the same or different amino acid sequence selected from the group consisting of SEQ ID NO: 3 to 81 . . . . In certain embodiments, the SIRP IgV monomers of the SIRP IgV multimer polyeptide comprise SIRPβ and/or SIRPγ IgV monomers, comprising an amino acid sequence selected from the group consisting of SEQ ID NO: 12 to 21, 31 to 33, 43 to 45, 55 to 57, 67 to 69, and 79 to 81. In certain embodiments, the SIRP IgV multimer polyeptide comprises an amino acid sequence selected from the group consisting of SEQ ID NO: 82 to 113, SEQ ID NO: 418 to 425 and SEQ ID NO: 430 to 435. In certain embodiments, the linker of the chimeric heavy chain comprises an amino acid sequence of GGGGSGGGGS. In certain embodiments, the first Fab domain binds to a surface receptor of an effector cell and the second Fab domain binds to an antigen associated with a diseased cell or tissue, or vice versa. In certain embodiment, the first Fab domain binds to a surface receptor of an effector cell selected from the group of effector cell surface receptors included in Table 4 and the second Fab domain binds to a disease associated antigen selected from the group of disease associated antigens included in Table 2 and 3, or vice versa. In certain embodiment, the first Fab domain is derived from an effector cell surface receptor-binding antibody included in Table 4 and the second Fab domain is derived from a disease associated antigen-binding antibody included in Table 2 and 3, or vice versa. In certain embodiment, the first Fab domain binds to a cell adhesion molecule and the second Fab domain binds to an antigen associated with a diseased cell or tissue, or vice versa. In certain embodiment, the first Fab domain binds to a cell adhesion molecule selected from the group of cell adhesion molecules included in Table 3 and the second Fab domain binds to a disease associated antigen selected from the group of disease associated antigens included in Table 2, or vice versa. In certain embodiment, the first Fab domain is derived from a cell adhesion molecule-binding antibody included in Table 3 and the second Fab domain is derived from a disease associated antigen-binding antibody included in Table 2, or vice versa. In certain embodiment, the first Fab domain binds to an antigen selected from the group consisting of CDH3, Nectin-4, EpCAM, ICAM-1, CEACAM5, and CD44, and the second Fab domain binds to an antigen selected from the group consisting of HER2, EGFR, VEGFR2, VEGFR1, VEGF, FGFR2, GD2, B7-H3, PD-L1, ROR1, CLDN18.2, CLDN6, CD70, CD117, CD38, SLAM7, BCMA, GPRC5D, FRHS, CD30, CD20, CD19, CD22, CD37 and CD52, or vice versa.

In some embodiments, an antibody fusion protein comprising a said fusion polypeptide comprising a SIRP IgV domain, is configured in a format wherein the antibody fusion protein comprises: 1) a first heavy chain comprising VH1-CH1-hinge-CH2-CH3 and a first light chain comprising VL1-CL, wherein the first heavy chain and light chain pair to form the first Fab domain; 2) a second chimeric heavy chain comprising from N-terminal to C-terminal VH2-CL-Hinge-CH2-CH3, and a second chimeric light chain comprising VL2-CH1, wherein the chimeric light chain pairs with the VH2-CL part of the chimeric heavy chain to form the 2^{nd} Fab domain; 3) wherein the first heavy chain and the chimeric heavy chain form a heterodimeric Fc; 4) a SIRP IgV monomer, wherein the SIRP IgV monomer is linked preferably through a linker to the N-terminal of the light chain, chimeric light chain, heavy chain or chimeric heavy chain; and 5) wherein the first and second Fab domains bind to two different non-CD47 antigens. In certain embodiments, the antibody fusion protein is configured in FV-132 as set forth in FIG. 9, wherein the SIRP IgV monomer is linked preferably through a linker to the N-terminal of the light chain comprising VL1-CL. In certain embodiments, the SIRP IgV monomer is a SIRPα, SIRPβ or SIRPγ IgV monomer. In certain embodiments, the SIRP IgV monomer comprises an amino acid sequence selected from the group consisting of SEQ ID NO: 3 to 81. In certain embodiments, the SIRP IgV monomer comprises a SIRPβ or SIRPγ IgV monomer. In certain embodiments, the SIRP IgV monomer comprises a SIRPβ or SIRPγ IgV monomer, comprising an amino acid sequence selected from the group consisting of SEQ ID NO: 12 to 21, 31 to 33, 43 to 45, 55 to 57, 67 to 69, and 79 to 81. In certain embodiments, the first Fab domain binds to a surface receptor of an effector cell and the second Fab domain binds to an antigen associated with a diseased cell or tissue, or vice versa. In certain embodiment, the first Fab domain binds to a surface receptor of an effector cell selected from the group of effector cell surface receptors included in Table 4 and the second Fab domain binds to a disease associated antigen selected from the group of disease associated antigens included in Table 2 and 3, or vice versa. In certain embodiment, the first Fab domain is derived from an effector cell surface receptor-binding antibody included in Table 4 and the second Fab domain is derived from a disease associated antigen-binding antibody included in Table 2 and 3, or vice versa. In certain embodiment, the first Fab domain binds to a cell adhesion molecule and the second Fab domain binds to an antigen associated with a diseased cell or tissue, or vice versa. In certain embodiment, the first Fab domain binds to a cell adhesion molecule selected from the group of cell adhesion molecules included in Table 3 and the second Fab domain binds to a disease associated antigen selected from the group of disease associated antigens included in Table 2, or vice versa. In certain embodiment, the first Fab domain is derived from a cell adhesion molecule-binding antibody included in Table 3 and the second Fab domain is derived from a disease associated antigen-binding antibody included in Table 2, or vice versa. In certain embodiment, the first Fab domain binds to an antigen selected from the group consisting of CDH3, Nectin-4, EpCAM, ICAM-1, CEACAM5, and CD44, and the second Fab domain binds to an antigen selected from the group consisting of HER2, EGFR, VEGFR2, VEGFR1, VEGF, FGFR2, GD2, B7-H3, PD-L1, ROR1, CLDN18.2, CLDN6, CD70, CD117, CD38, SLAM7, BCMA, GPRC5D, FcRH5, CD30, CD20, CD19, CD22, CD37 and CD52, or vice versa.

In some embodiments, an antibody fusion protein comprising a said fusion polypeptide comprising a SIRP IgV domain, is configured in a format wherein the antibody fusion protein comprises: 1) a first heavy chain comprising VH1-CH1-binge-CH2-CH3 and a first light chain comprising VL1-CL, wherein the first heavy chain and light chain pair to form the first Fab domain; 2) a second chimeric heavy chain comprising from N-terminal to C-terminal VH2-CL-Hinge-CH2-CH3, and a second chimeric light chain comprising VL2-CH1, wherein the chimeric light chain pairs with the VH2-CL part of the chimeric heavy chain to form the 2^{nd} Fab domain; 3) wherein the first heavy chain and the chimeric heavy chain form a heterodimeric Fc; 4) a SIRP IgV multimer polypeptide comprising two, three or four SIRP IgV monomers, wherein the SIRP IgV multimer polypeptide is linked preferably through a linker to the N-terminal of the light chain, chimeric light chain, heavy chain and/or chimeric heavy chain; and 5) wherein the first and second Fab domains bind to two different non-CD47 antigens. In certain embodiments, the antibody fusion protein is configured in FV-133, 134, or 135 as set forth in FIG. 9, wherein the SIRP IgV multimer polypeptide is linked preferably through a linker to the N-terminal of the light chain comprising VL1-CL. In certain embodiments, the SIRP IgV monomers of the SIRP IgV multimer polyeptide comprise SIRPα, SIRPβ and/or SIRPγ IgV monomers. In certain embodiments, the SIRP IgV monomers of the SIRP IgV multimer polyeptide comprise the same or different amino acid sequence selected from the group consisting of SEQ ID NO: 3 to 81. In certain embodiments, the SIRP IgV monomers of the SIRP IgV multimer polyeptide comprise SIRPβ and/or SIRPγ IgV monomers, comprising an amino acid sequence selected from the group consisting of SEQ ID NO: 12 to 21, 31 to 33, 43 to 45, 55 to 57, 67 to 69, and 79 to 81 In certain embodiments, the SIRP IgV multimer polyeptide comprises an amino acid sequence selected from the group consisting of SEQ ID NO: 82 to 113, SEQ ID NO: 418 to 425 and SEQ ID NO: 430 to 435. In certain embodiments, the first Fab domain binds to a surface receptor of an effector cell and the second Fab domain binds to an antigen associated with a diseased cell or tissue, or vice versa. In certain embodiment, the first Fab domain binds to a surface receptor of an effector cell selected from the group of effector cell surface receptors included in Table 4 and the second Fab domain binds to a disease associated antigen selected from the group of disease associated antigens included in Table 2 and 3, or vice versa. In certain embodiment, the first Fab domain is derived from an effector cell surface receptor-binding antibody included in Table 4 and the second Fab domain is derived from a disease associated antigen-binding antibody included in Table 2 and 3, or vice versa. In certain embodiment, the first Fab domain binds to a cell adhesion molecule and the second Fab domain binds to an antigen associated with a diseased cell or tissue, or vice versa. In certain embodiment, the first Fab domain binds to a cell adhesion molecule selected from the group of cell adhesion molecules included in Table 3 and the second Fab domain binds to a disease associated antigen selected from the group of disease associated antigens included in Table 2, or vice versa. In certain embodiment, the first Fab domain is derived from a cell adhesion molecule-binding antibody included in Table 3 and the second Fab domain is derived from a disease associated antigen-binding antibody included in Table 2, or vice versa. In certain embodiment, the first Fab domain binds to an antigen selected from the group consisting of CDH3, Nectin-4, EpCAM, ICAM-1, CEACAM5, and CD44, and the second Fab domain binds to an antigen selected from the group consisting of HER2, EGFR, VEGFR2, VEGFR1, VEGF, FGFR2, GD2, B7-H3, PD-L1, ROR1, CLDN18.2, CLDN6, CD70, CD117, CD38, SLAM7, BCMA, GPRC5D, FcRH5, CD30, CD20, CD19, CD22, CD37 and CD52, or vice versa.

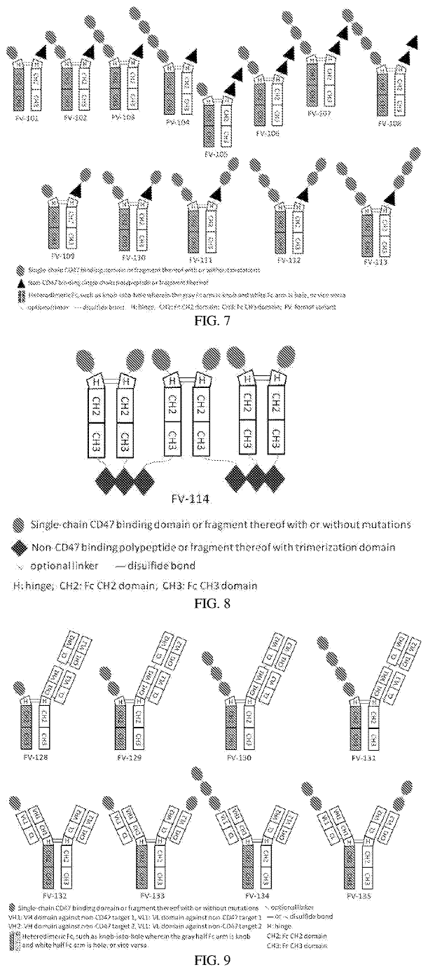

In some embodiments, a Fc fusion protein comprising a said fusion polypeptide comprising a SIRP IgV domain and a heterodimeric Fc comprising hinge-CH2-CH3 is configured in FV-101 as set forth in FIG. 7, wherein one SIRP IgV monomer is directly linked to the N-terminal of one Fc chain, and one single-chain polypeptide binding to a non-CD47 target is linked preferably through a linker to the N-terminal of the other Fc chain of the heterodimeric Fc. In certain embodiments, the SIRP IgV monomer is a SIRPα, SIRPβ or SIRPγ IgV monomer. In certain embodiments, the SIRP IgV monomer comprises an amino acid sequence selected from the group consisting of SEQ ID NO: 3 to 81. In certain embodiments, the SIRP IgV monomer comprises a SIRPβ or SIRPγ IgV monomer. In certain embodiments, the SIRP IgV monomer comprises a SIRPβ or SIRPγ IgV monomer, comprising an amino acid sequence selected from the group consisting of SEQ ID NO: 12 to 21, 31 to 33, 43 to 45, 55 to 57, 67 to 69, and 79 to 81. In some embodiments, the non-CD47 binding single chain domain of the fusion polypeptide comprises a peptide, a receptor or fragment thereof, a ligand or fragment thereof, a cytokine or fragment thereof, or a chemokine or fragment thereof, a growth factor or fragment thereof. In some embodiments, the non-CD47 binding single chain domain of the fusion polypeptide comprises an amino acid sequence at least 60%, at least 70%, at least 80%, at least 85% or at least 90% identical to a sequence selected from a group of exemplary peptides and polypeptides as set forth in Table 5. In some embodiments, the SIRP monomer of the fusion protein configured in FV-101 comprises a mutation of K53H, and the non-CD47 binding single chain domain of the fusion protein comprises a VHH domain or scFv comprising a set of VH and/or VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the fusion protein configured in FV-101 comprises a mutation of R69H, and the non-CD47 binding single chain domain of the fusion protein comprises a VHH domain or scFv comprising a set of VH and/or VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the fusion protein configured in FV-101 comprises a mutation of Q52H, and the non-CD47 binding single chain domain of the fusion protein comprises a VHH domain or scFv comprising a set of VH and/or VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4. In some embodiments, the SIRP monomer of the fusion protein configured in FV-101 comprises a mutation of K68H, and the non-CD47 binding single chain domain of the fusion protein comprises a VHH domain or scFv comprising a set of VH and/or VL CDR sequences at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4.

In some embodiments, a Fc fusion protein comprising a said fusion polypeptide comprising a SIRP IgV domain and a heterodimeric Fc comprising hinge-CH2-CH3 is configured in FV-102, 103 or 104 as set forth in FIG. 7, wherein a SIRP IgV multimer polypeptide of present disclosure comprising two, three or four SIRP IgV monomers is directly linked to the N-terminal of one Fc chain, and one single-chain polypeptide binding to a non-CD47 target is linked preferably through a linker to the N-terminal of the other Fc chain of the heterodimeric Fc. In certain embodiments, the two, three or four SIRP IgV monomers of the SIRP IgV multimer polypeptide comprise SIRPα, SIRPβ and/or SIRPγ IgV monomers. In certain embodiments, the two, three or four SIRP IgV monomers of the SIRP IgV multimer polypeptide comprise the same or different amino acid sequence and/or mutation. In certain embodiments, the two, three or four SIRP IgV monomers of the SIRP IgV multimer polypeptide comprise the same or different amino acid sequence selected from the group consisting of SEQ ID NO: 3 to 81. In certain embodiments, the two, three of four SIRP IgV monomers of the SIRP IgV multimer polypeptide from the N-terminal monomer to the C-terminal monomer comprise substitution as described for such SIRP IgV multimer polypeptides earlier in present disclosure. In some embodiments, the non-CD47 binding single chain domain of the fusion polypeptide comprises a peptide, a receptor or fragment thereof, a ligand or fragment thereof, a cytokine or fragment thereof, or a chemokine or fragment thereof, a growth factor or fragment thereof. In some embodiments, the non-CD47 binding single chain domain of the fusion polypeptide comprises an amino acid sequence at least 60%, at least 70%, at least 80%, at least 85% or at least 90% identical to a sequence selected from a group of exemplary peptides and polypeptides as set forth in Table 5. In some embodiments, the non-CD47 binding single chain domain of the fusion protein comprises a VHH domain or scFv comprising a set of VH and/or VL CDR sequences at least 30%, at least 40%, at least 50%, at least 60%, at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4.

In some embodiments, a Fc fusion protein comprising a said fusion polypeptide comprising a SIRP IgV domain and a heterodimeric Fc comprising binge-CH2-CH3, is configured in FV-109 as set forth in FIG. 7, wherein one SIRP IgV monomer is directly linked to the N-terminal of one Fc chain, and a second SIRP IgV monomer is linked preferably through a linker to the N-terminal of a non-CD47 binding single-chain polypeptide which is further linked preferably through a linker to the N-terminal of the other Fc chain of the heterodimeric Fc. In certain embodiments, the two SIRP IgV monomers of the Fe fusion protein comprise SIRPα, SIRPβ and/or SIRPγ IgV monomers. In certain embodiments, the two SIRP IgV monomers of the Fe fusion protein comprise the same or different amino acid sequence and/or mutation. In certain embodiments, the two SIRP IgV monomers of the Fc fusion protein comprise the same or two different amino acid sequences selected from the group consisting of SEQ ID NO: 3 to 81. In certain embodiments, the SIRP IgV monomer directly linked to the N-terminal of one Fc chain comprises a SIRPβ or SIRPγ IgV monomer. In certain embodiments, the SIRP IgV monomer directly linked to the N-terminal of one Fc chain comprises a SIRPβ or SIRPγ IgV monomer comprising an amino acid sequence selected from the group consisting of SEQ ID NO: 12 to 21, 31 to 33, 43 to 45, 55 to 57, 67 to 69, and 79 to 81. In certain embodiments, the SIRP IgV monomer directly linked to the N-terminal of one Fe chain comprises a SIRPα IgV monomer comprising an amino acid sequence selected from the group consisting of SEQ ID NO: 3 to 11, 22 to 30, 34 to 42, 46 to 54, 58 to 66, and 70 to 78. In certain embodiments, the SIRP IgV monomer directly linked to the N-terminal of one Fc chain comprises a SIRPα IgV monomer and the SIRP IgV monomer linked to the N-terminal of the non-CD47 binding single-chain polypeptide comprises a SIRPα, SIRPβ or SIRPγ IgV monomer. In some embodiments, the non-CD47 binding single chain domain of the fusion polypeptide comprises a peptide, a receptor or fragment thereof, a ligand or fragment thereof, a cytokine or fragment thereof, or a chemokine or fragment thereof, a growth factor or fragment thereof. In some embodiments, the non-CD47 binding single chain domain of the fusion polypeptide comprises an amino acid sequence at least 60%, at least 70%, at least 80%, at least 85% or at least 90% identical to a sequence selected from a group of exemplary peptides and polypeptides as set forth in Table 5. In some embodiments, the non-CD47 binding single chain domain of the fusion protein comprises a VHH domain or scFv comprising a set of VH and/or VL CDR sequences at least 30%, at least 40%, at least 50%, at least 60%, at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4.

In some embodiments, a Fc fusion protein comprising a said fusion polypeptide comprising a SIRP IgV domain and a heterodimeric Fc comprising hinge-CH2-CH3 is configured in FV-111, 112 or 113 as set forth in FIG. 7, wherein one SIRP IgV multimer polypeptide of present disclosure comprising two or three SIRP IgV monomers is directly linked to the N-terminal of one Fc chain, and a second SIRP IgV multimer polypeptide of present disclosure comprising two or three SIRP IgV monomers is linked preferably through a linker to the N-terminal of a non-CD47 binding single-chain polypeptide which is further linked preferably through a linker to the N-terminal of the other Fc chain of the heterodimeric Fc. In certain embodiments, the SIRP IgV monomers of the Fc fusion protein comprise SIRPα, SIRPβ and/or SIRPγ IgV monomers. In certain embodiments, the SIRP IgV monomers of the Fc fusion protein comprise the same or different amino acid sequence and/or mutation. In certain embodiments, the SIRP IgV monomers of the Fc fusion protein comprise the same or two different amino acid sequences selected from the group consisting of SEQ ID NO: 3 to 81. In certain embodiments, the SIRP IgV monomers directly linked to the N-terminal of one Fc chain comprises a SIRP or SIRPγ IgV monomer. In certain embodiments, the SIRP IgV monomer directly linked to the N-terminal of one Fc chain comprises a SIRPβ or SIRPγ IgV monomer comprising an amino acid sequence selected from the group consisting of SEQ ID NO: 12 to 21, 31 to 33, 43 to 45, 55 to 57, 67 to 69, and 79 to 81. In certain embodiments, the SIRP IgV monomers directly linked to the N-terminal of one Fc chain comprises a SIRPα IgV monomer comprising an amino acid sequence selected from the group consisting of SEQ ID NO: 3 to 11, 22 to 30, 34 to 42, 46 to 54, 58 to 66, and 70 to 78. In certain embodiments, the SIRP IgV monomers directly linked to the N-terminal of one Fc chain comprise SIRPα IgV monomers and the SIRP IgV monomers linked to the N-terminal of the non-CD47 binding single-chain polypeptide comprises SIRPα, SIRPβ and/or SIRPγ IgV monomers. In some embodiments, the non-CD47 binding single chain domain of the fusion polypeptide comprises a peptide, a receptor or fragment thereof, a ligand or fragment thereof, a cytokine or fragment thereof, or a chemokine or fragment thereof, a growth factor or fragment thereof. In some embodiments, the non-CD47 binding single chain domain of the fusion polypeptide comprises an amino acid sequence at least 60%, at least 70%, at least 80%, at least 85% or at least 90% identical to a sequence selected from a group of exemplary peptides and polypeptides as set forth in Table 5. In some embodiments, the non-CD47 binding single chain domain of the fusion protein comprises a VHH domain or scFv comprising a set of VH and/or VL CDR sequences at least 30%, at least 40%, at least 50%, at least 60%, at least 70%, at least 75%, at least 80%, at least 85%, at least 90% or at least 95% identical to the set of VH and VL CDR sequences according to the IMGT numbering scheme of an antibody selected from a group of exemplary antibodies as set forth in Table 2 to 4.

In some embodiments, an antibody fusion protein comprising a said fusion polypeptide comprising a SIRP IgV domain, is configured in a format wherein the antibody fusion protein comprising: 1) a first polypeptide comprising a single-chain domain binding to a first non-CD47 antigen, wherein the single-chain domain is linked preferably through a linker to the N-terminal of a Fc region, 2) an antibody heavy chain, wherein the Fc region of the heavy chain forms a heterodimeric Fc with the Fc region of the first polypeptide, 3) an antibody light chain, wherein the light chain pairs with the heavy chain to form a Fab domain that binds to a second non-CD47 antigen, and 4) a SIRP IgV domain comprising one, two, three, four or more SIRP IgV monomers, wherein the SIRP IgV domain is linked preferably through a linker to the N-terminal of the light chain and/or heavy chain. In certain embodiments, the antibody fusion protein is configured in FV-172 to 182 as set forth in FIG. 12. In certain embodiments, the SIRP IgV monomers of the SIRP IgV domain comprise SIRPα, SIRPβ and/or SIRPγ IgV monomers. In certain embodiments, the SIRP IgV monomers comprise the same or different amino acid sequence and/or mutation. In certain embodiments, the SIRP IgV monomers comprise the same or different amino acid sequences selected from the group consisting of SEQ ID NO: 3 to 81. In certain embodiments, the Fab domain binds to a surface receptor of an effector cell and the single-chain domain binds to an antigen associated with a diseased cell or tissue, or vice versa. In certain embodiment, the Fab domain is derived from an effector cell surface receptor-binding antibody included in Table 4 and the single-chain domain is derived from a disease associated antigen-binding polypeptide included in Table 5. In certain embodiment, the Fab domain is derived from a disease associated antigen-binding antibody included in Table 2 and 3 and the single-chain domain is derived from an effector cell surface receptor-binding polypeptide included in Table 5. In certain embodiment, the Fab domain binds to a surface receptor of an effector cell and the single-chain domain binds to a cell adhesion molecule, or vice versa. In certain embodiment, the Fab domain binds to a cell adhesion molecule included in Table 3 and the single-chain domain binds to a disease associated antigen selected from the group of disease associated antigens included in Table 2, or vice versa. In certain embodiment, the Fab domain is derived from a cell-adhesion-molecule-binding antibody included in Table 3 and the single-chain domain is derived from a disease associated antigen-binding polypeptide included in Table 5.

In certain embodiments, the heterodimeric Fc of FV-1, 64 to 78, 101 to 104, 109 to 113, 128 to 135, 150 to 190 above, comprises a human IgG1, human IgG4 or human IgG2 Fc, with wild type, reduced, or abolished effector function. In certain embodiments, the two heterodimeric Fc chains of FV-1, 64 to 78, 101 to 104, 109 to 113, 128 to 135, 150 to 190 above, comprises a pair of amino acid sequence selected from the group consisting of SEQ ID NO: 136:137, 138:139, 140:141 and 142:143, wherein “:” indicates pairing of the two sequences from the left to right of “:” symbol. In certain embodiments, the heterodimeric Fc comprises a human IgG1 Fe, comprising a pair of amino acid sequence selected from the group consisting of SEQ ID NO: 136:137. In certain embodiments, the hoterodimeric Fc comprises a human IgG1 Fe with enhanced effector function, comprising mutations known in the arts to enhance the effector function of human IgG1 Fc. In certain embodiments, the Fc region is a human IgG1 Fc with enhanced antibody-dependent cellular phagocytosis (ADCP) function, comprising a mutation selected from the group consisting of G236A, S239D/1332E, G236A/S239D/1332E. S239D/A330L/1332E, G236A/A330L/1332E, G236A/S239D/A330L/1332E, and F243L/R292P/Y300L/V305I/P396L, according to the EU numbering scheme.

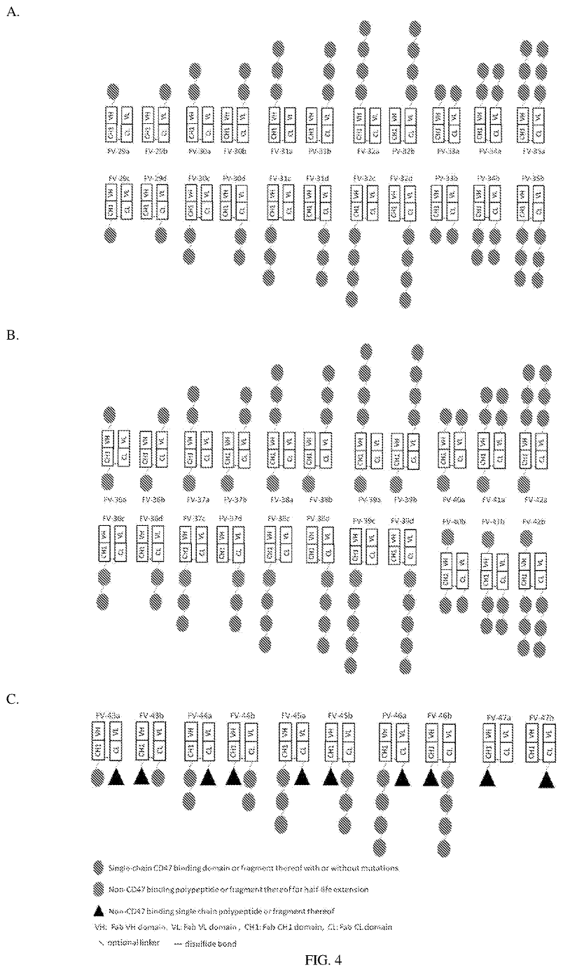

In some embodiments, a Fab fusion protein comprising a said fusion polypeptide comprising a SIRP IgV domain is configured in a format selected from the group consisting of FV-29a to 35b as set forth in FIG. 4, wherein the protein comprising a SIRP IgV domain and a Fab domain comprising a Fd chain comprising VH-CH1 and a light chain comprising VL-CL, wherein the SIRP IgV domain is connected to the N-terminal or the C-terminal of the Fd chain and/or light chain of the Fab domain. In certain embodiments, the SIRP IgV domain of the Fab fusion protein comprises one SIRPα, SIRPβ or SIRPγ IgV monomer and is configured in FV-29a, 29b, 29c or 29d as set forth in FIG. 4. In certain embodiments, the SIRP IgV domain of the Fab fusion protein comprises one SIRP IgV monomer comprising an amino acid sequence selected from the group consisting of SEQ ID NO: 3 to 81. In certain embodiments, the SIRP IgV domain of the Fab fusion protein comprises two, three, four or more SIRP IgV monomers, wherein the two, three, four or more SIRP IgV monomers comprise SIRPα, SIRPβ and/or SIRPγ IgV monomers. In certain embodiments, the SIRP IgV domain of the Fab fusion protein comprises two, three, four or more SIRP IgV monomers comprising the same or different amino acid sequences selected from the group consisting of SEQ ID NO: 3 to 81. In some embodiments, a Fab fusion protein comprises a Fab domain and a single-chain domain, wherein the single-chain domain linked preferentially through a linker to the N-terminal or the C-terminal of the Fd chain or light chain of the Fab domain. In certain embodiments, the Fab fusion protein comprising the single-chain domain is configured in a format of FV-47a or b, as set forth in FIG. 4, wherein the single-chain domain binds to an antigen associated with a diseased tissue such as cancer or fibrotic disease. In certain embodiments, the Fab domain of the Fab fusion protein binds to a surface antigen on an effector cell. In certain embodiments, the single-chain third antigen-binding domain of the Fab fusion protein, binds to an antigen associated with a diseased tissue. In certain embodiments, the Fab domain of the Fab fusion proteins binds to a surface antigen on a phagocyte effector cell selected from the group consisting of Dectin-1, Dectin-2, Dectin-3, Mincle, CLEC5A, CLEC2, DCL-1 (CLEC13A), DC-SIGN, DNGR-1 (CLEC9A), CD91, LOX-1, CD205, CD206, CD89, TREM1, MARCO, CD36, CD14, CD44, CD40, CLEVER-1, PSGL-1, VSIG4, Toll-like receptor (TLR), chemokine receptor, cytokine receptor, MerTK, Tyro3, AXL, Siglec-1, Siglect-14, Siglect-16, LILRA1, LILRA2, LILRA4, LILRA5 and LILRA. In certain embodiments, the Fab domain of the Fab fusion protein binds to Dectin-1, CLEC5A or MerTK. In certain embodiments, the Fab domain of the Fab fusion proteins binds to a hapten antigen selected from the group consisting of a chelator and a peptide histamine-succinyl-glycine (HSG). In further embodiments, the chelator is a macrocyclic chelator, an acyclic chelator or a hybrid chelator. In additional embodiments, the chelator is selected from the group consisting of 1,4,7,10-Tetraazacyclododecane-1,4,7,10-tetraacetic acid (DOTA), DOTATATE, DOTA-Bn, DO2A, Diethylenetriamene pentaacetate (DTPA), 6-amino-1,4-diazepine-triacetate (DATA), PCTA, 1,4,7-triazacyclononane-N,N′,N″-triacetic acid (NOTA), NOTP, TRAP, 1,4,7-Triazacyclononane (TACN), 6-amino-6-methylperhydro-1,4-diazepinetetraacetic acid (AAZTA), H4pypa, H2dedpa, H2CHXhox, H2hox, HBED and DFO, as described in (Sneddon et al., Curr Opin Chem Biol, 2021). In some embodiments, the Fab fusion protein comprising a SIRP IgV domain and a Fab domain additionally comprises a third antigen-binding domain, wherein the third antigen-binding domain is a single-chain polypeptide binding to a third antigen and is linked preferentially through a linker to the N-terminal or the C-terminal of the SIRP IgV domain or to the N-terminal or the C-terminal of the Fd chain or light chain of the Fab domain. In certain embodiments, the Fab fusion protein comprising the third antigen-binding domain is configured in a format selected from the group consisting of FV-36a to 46b as set forth in FIG. 4. In certain embodiments, the Fab fusion protein comprises a third antigen-binding domain, wherein the third antigen-binding domain comprises a single-chain domain that binds to a third antigen associated with a diseased tissue. In certain embodiments, the single-chain third antigen-binding domain of the Fab fusion protein binds to a tumor associated antigen (TAA). In certain embodiments, the Fab fusion protein comprising the third antigen-binding domain is configured in a format selected from the group consisting of FV-43a to 46b as set forth in FIG. 4. In certain embodiments, the Fab domain comprises a set of VH and/or VL CDR sequences at least 70%, 75%, 80%, 85%, 90%, or 95% identical to the set of VH and/or VL CDR sequences according to the IMGT numbering scheme (The Immunologist, 7, 132-136 (1999); Dev Comp Immunol. 2003 January; 27 (1): 55-77) from an antibody selected from the group of antibodies consisting of anti-Dectin 1 antibody 2M24 (WO2022077006A1) and 15E2 (WO2008118587A3), anti-Dectin 2 antibody BDC-3042, anti-CLEC5A antibody 1F7 (MAbs. 2022 January-December; 14 (1): 2040083), anti-MerTK antibody 18G7 (WO2020214995A1) and RGX-019, anti-CD205 antibody 3G9 (WO2009061996) and OBT076, anti-CD206 antibody CDX-1307, anti-DC-SIGN antibody hDIVI (U.S. Pat. No. 8,067,167B2), anti-DOTA antibody 2D12.5 (Corneillie et al., J Am Chem Soc, 2003), C825 (U.S. Pat. No. 8,648,176), huC825 (US20200140543A1) and hLL2 (US20030124057A1), anti-DTPA antibody Mab 734 (US2003016270941), and anti-HSG antibody Mab 679 (US20030162709A1). In certain embodiments, the Fab fusion protein comprises a third antigen-binding domain, wherein the third antigen-binding domain comprises a single-chain domain that binds to human albumin (HSA). In certain embodiments, the Fab fusion protein comprising a HSA-binding third domain is configured in a format selected from the group consisting of FV-36a to 42b as set forth in FIG. 4. In certain embodiments, the HSA-binding third domain comprises an amino acid sequence of SEQ ID NO: 214. In some embodiments, a fusion protein configured in a format of FV-29 to 46 as set forth in FIG. 4, comprises one, two, three, four or six SIRP IgV monomers comprising the same or different mutation selected from the group consisting of K53H, R69H, Q52H, K68H, Q52H+K68H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a fusion protein configured in FV-32, 34, 43 or 44 selected from the group of proteins consisting of MP-87, MP-88, MP-89, MP-90, MP-92 and MP-93 as set forth in Table 21, exhibits higher binding to CD47 at an acidic pH than at physiological pH, whereas a protein configured in FV-43 as MP-91 does not exhibit higher binding to CD47 at an acidic pH than at physiological pH.

In some embodiments, a protein configured in FV-29, 36 or 43 as set forth in FIG. 4, or FV-64, 65, 66, 67, 79, 93 or 97 as set forth in FIG. 6, or FV-101 or 105 as set forth in FIG. 7, FV-128 or 132 as set forth in FIG. 9, or FV-150, 151, 152, 153, 154, 155 or 156 as set forth in FIG. 11, or FV-168, 172 or 176 as set forth in FIG. 12, comprises one SIRP IgV monomer comprising a mutation selected from the group consisting of K53H, R69H, Q52H, K68H and Q52H+K68H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH.

In some embodiments, a protein configured in FV-2, 8 or 9 as set forth in FIG. 2, or FV-33 or 40 as set forth in FIG. 4, or FV-48, 49, 58 or 61 as set forth in FIG. 5, or FV-71, 72, 83 or 84 as set forth in FIG. 6, or FV-109 as set forth in FIG. 7, or FV-133 as set forth in FIG. 9, or FV-136, 137, 143 or 144 as set forth in FIG. 10, or FV-160 or 161 as set forth in FIG. 11, or FV-180 as set forth in FIG. 12, comprises two SIRP IgV monomers comprising the same or different mutation selected from the group consisting of K53H, R69H, Q52H, K68H, Q52H+K68H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH.

In some embodiments, a protein configured in FV-3, 4, 6, 7, 10 or 11 as set forth in FIG. 2, or FV-30, 31, 32, 34, 37, 38, 39, 41, 42, 44, 45 or 46 as set forth in FIG. 4, or FV-50, 51, 52, 53, 55, 56, 57, 59, 60, 62 or 63 as set forth in FIG. 5, or FV-68, 69, 70, 74, 75, 76, 77, 78, 80, 81, 82, 86-90, 94, 95, 96, 98, 99 or 100 as set forth in FIG. 6, or FV-102, 103, 104, 106, 107, 108, 110, 111, 112 or 113 as set forth in FIG. 7, or FV-129, 130, 131, 134 or 135 as set forth in FIG. 9, FV-138, 139, 140, 141, 145, 146, 147 or 148 as set forth in FIG. 10, or FV-157, 158, 159, 163, 164, 165 or 167 as, set forth in FIG. 11, or FV-169, 170, 171, 173, 174, 175, 177, 178, 179, 181 or 182 as set forth in FIG. 12, comprises a SIRP IgV multimer polypeptide comprising two, three or four SIRP IgV monomers comprising the same mutation selected from the group consisting of K53H, R69H, K53H+K68H, R69H+K68H, K53H+Q52H and R69H+Q52H, exhibits higher binding to CD47 at an acidic pH than at physiological pH, whereas an otherwise identical protein except without any of the mutation K53H, R69H, Q52H and K68H, does not exhibit higher binding to CD47 at an acidic pH than at physiological pH, and an otherwise identical protein except comprising a mutation of M72H, V33H or G97H instead, does not exhibit higher binding to CD47 at an acidic pH than at physiological pH. In some embodiments, a protein configured in FV-3, 4, 6, 7, 10 or 11 as set forth in FIG. 2, or FV-30, 31, 32, 34, 37, 38, 39, 41, 42, 44, 45 or 46 as set forth in FIG. 4, or FV-50, 51, 52, 53, 55, 56, 57, 59, 60, 62 or 63 as set forth in FIG. 5, or FV-68, 69, 70, 74, 75, 76, 77, 78, 80, 81, 82, 86-90, 94, 95, 96, 98, 99 or 100 as set forth in FIG. 6, or FV-102, 103, 104, 106, 107, 108, 110, 111, 112 or 113 as set forth in FIG. 7, or FV-129, 130, 131, 134 or 135 as set forth in FIG. 9, FV-138, 139, 140, 141, 145, 146, 147 or 148 as set forth in FIG. 10, or FV-157, 158, 159, 163, 164, 165 or 167 as, set forth in FIG. 11, or FV-169, 170, 171, 173, 174, 175, 177, 178, 179, 181 or 182 as set forth in FIG. 12, comprises a SIRP IgV multimer polypeptide comprising two, three or four SIRP IgV monomers wherein at least two of the SIRP IgV monomers comprise different mutation selected from the group consisting of K53H, R69H, Q52H, K68H, Q52H+K68H, K53H+K68H, R69H+K68H, K53H+Q52H and R69H+Q52H, and at most one of the SIRP IgV monomers comprises a mutation of Q52H, K68H or Q52H+K68H, exhibits higher binding to CD47 at an acidic pH than at physiological pH. In some embodiments, a protein configured in FV-3, 4, 6, 7, 10 or 11 as set forth in FIG. 2, or FV-30, 31, 32, 34, 37, 38, 39, 41, 42, 44, 45 or 46 as set forth in FIG. 4, or FV-50, 51, 52, 53, 55, 56, 57, 59, 60, 62 or 63 as set forth in FIG. 5, or FV-68, 69, 70, 74, 75, 76, 77, 78, 80, 81, 82, 86-90, 94, 95, 96, 98, 99 or 100 as set forth in FIG. 6, or FV-102, 103, 104, 106, 107, 108, 110, 111, 112 or 113 as set forth in FIG. 7, or FV-129, 130, 131, 134 or 135 as set forth in FIG. 9, FV-138, 139, 140, 141, 145, 146, 147 or 148 as set forth in FIG. 10, or FV-157, 158, 159, 163, 164, 165 or 167 as, set forth in FIG. 11, or FV-169, 170, 171, 173, 174, 175, 177, 178, 179, 181 or 182 as set forth in FIG. 12, comprises a SIRP IgV multimer polypeptide comprising an amino acid sequence selected from the group consisting of SEQ ID NO: 82 to 113, SEQ ID NO: 418 to 425 and SEQ ID NO: 430 to 435, exhibits higher binding to CD47 at an acidic pH than at physiological pH. In some embodiments, a protein configured in FV-3, 4, 6, 7, 10 or 11 as set forth in FIG. 2, or FV-30, 31, 32, 34, 37, 38, 39, 41, 42, 44, 45 or 46 as set forth in FIG. 4, or FV-50, 51, 52, 53, 55, 56, 57, 59, 60, 62 or 63 as set forth in FIG. 5, or FV-68, 69, 70, 74, 75, 76, 77, 78, 80, 81, 82, 86-90, 94, 95, 96, 98, 99 or 100 as set forth in FIG. 6, or FV-102, 103, 104, 106, 107, 108, 110, 111, 112 or 113 as set forth in FIG. 7, or FV-129, 130, 131, 134 or 135 as set forth in FIG. 9, FV-138, 139, 140, 141, 145, 146, 147 or 148 as set forth in FIG. 10, or FV-157, 158, 159, 163, 164, 165 or 167 as, set forth in FIG. 11, or FV-169, 170, 171, 173, 174, 175, 177, 178, 179, 181 or 182 as set forth in FIG. 12, comprises a SIRP IgV multimer polypeptide comprising an amino acid sequence selected from the group consisting of SEQ ID NO: 418 to 425 and SEQ ID NO: 430 to 435, exhibits higher binding to CD47 at an acidic pH than at physiological pH, whereas a protein in the same format comprises a SIRP IgV multimer polypeptide comprising an amino acid sequence selected from the group consisting of SEQ ID NO: 426 to 429 and SEQ ID NO: 436 to 439, does not exhibit higher binding to CD47 at an acidic pH than at physiological pH.

In some embodiments, the linker of the protein for example as configured in an exemplary format described in FV-3 to 6, 29a to 47b, 48 to 78, 101 to 104, 109 to 113, and 128 to 190 above, comprises an amino acid sequence of (GGGGS)n, wherein n=1, 2, 3, 4, 5, or 6. In some embodiments, the linker of the protein for example as configured in an exemplary format described in FV-3 to 6, 29a to 47b, 48 to 78, 101 to 104, 109 to 113, and 128 to 190 above, comprises an amino acid sequence of GGGGSGGGGS (SEQ ID NO: 119), GGGGSGGGGSGGGGS (SEQ ID NO: 120) or GGGGSGGGGSGGGGSGGGGS (SEQ ID NO: 121).

In some embodiments, the Fab domain of the antibody fusion protein or Fab fusion protein, for example as configured in an exemplary format described in FV-29a to 47b, 48 to 78, and 128 to 190 above, binds to a non-CD47 antigen. In some embodiments, the Fab domain binds to a soluble antigen, an aggregate of antigen, a surface antigen on a cell, a surface antigen on a microbe, or a hapten. In some embodiments, the non-CD47 binding Fab domain binds to a surface antigen on a diseased cell, an infected cell or an effector cell. In certain embodiments, the diseased cell is a cancer cell, a fibrotic cell, an atherosclerotic cell, an inflammatory cell or a senescent cell. In certain embodiments, the infected cell is a cell infected by bacterium, fungus, virus and/or parasite. In certain embodiments, the effector cell is a myeloid cell, a lymphocyte or a granulocyte. In certain embodiments, the effector cell is a macrophage, a monocyte, a dendritic cell, a B cell, a T cell, a NK cell, a NKT cell, an innate lymphoid cell (ILC), a neutrophil, and/or a mast cell. In certain embodiments, the non-CD47 binding Fab domain binds to a microbe, wherein the microbe is a bacterium, a fungus, a protozoan, or a virus. In certain embodiments, the non-CD47 binding Fab domain binds to a hapten, wherein the hapten comprises a chelator that binds to a radionuclide or radiometal. In certain embodiments, the radionuclide is selected from the group consisting of ²²⁵Ac, ²¹¹At, ²¹²Bi, ⁶²Cu, ⁶⁴Cu, ⁶⁷Cu, 66Ga, ⁶⁷Ga, ⁶⁸Ga, ¹²³I, ¹²⁵I, ¹³¹I, ¹¹¹In, ¹⁷⁷Lu, ²¹²Pb, ¹⁸⁶Re, ¹⁸⁸Re, ⁴⁴⁵C ¹⁴⁹Tb. ¹⁵²Tb, ¹⁵Tb, ¹⁶Th, Y and Zr. In certain embodiments, the chelator is a macrocyclic chelator, an acyclic chelator or a hybrid chelator. In additional embodiments, the chelator is selected from the group consisting of 1,4,7,10-Tetraazacyclododecane-1,4,7,10-tetraacetic acid (DOTA), DOTATATE, DOTA-Bn, DO2A, Diethylenetriamene pentaacetate (DTPA), 6-amino-1,4-diazepine-triacetate (DATA), PCTA, 1,4,7-triazacyclononane-N,N′,N″-triacetic acid (NOTA), NOTP, TRAP, 1,4,7-Triazacyclononane (TACN), 6-amino-6-methylperhydro-1,4-diazepinetetraacetic acid (AAZTA), H4pypa, H2dedpa, H2CHXhox, H2hox, HBED and DFO, as described in (Sneddon et al., Curr Opin Chem Biol, 2021). In certain embodiments, the hapten comprises a hapten peptide histamine-succinyl-glycine (HSG).

In some embodiments, the non-CD47 binding Fab domain of the antibody fusion protein or Fab fusion protein, for example as configured in an exemplary format described in FV-29a to 47b, 48 to 78, and 128 to 190 above, binds to an antigen selected from the group consisting of CD38, SLAMF7, BCMA, GPRC5D, FcRH5, CD138, CD56, CD74, CD26, CD46, CD19, CD20, CD22, CD30, CD33, CD37, CD70, CD117, CD79b, CD123, CD52, CD98, CD205, PD-L1, HER2, EGFR, VEGFR2, VEGFR1, VEGF, CD93, TGFβ, IL-6, IL-6R, IL-8, GDF-15, GFRAL, FOFR2, FGFR3, FGFR4, FGFR1, GD2, GD3, B7-H3, B7-H4, phosphatidylserine, DR5, DR4, DR3, TL1A, CD95 (Fas), TNFR1, TNFR2, CD24, CD31, CD61, CD200, SLAMF3, SLAMF4, HLA-G, HLA-E, HLA-DR, NKG2D ligands (MICA, MICB, ULBP1-6), CD1d-presented lipid antigen, P-Cadherin, N-Cadherin, E-Cadherin, VE-Cadherin, R-cadherin (CDH4), K-cadherin (CDH6), VE-cadherin (CDH5), H-cadherin (CDH13), OB-cadherin, LI-cadherin (CDH17), CDH11, CDH19, CLDN18.2, CLDN6, CLDN3, CLDN4, CLDN7, CLDN1, CLDN2, CLDN5, CLDN8, CLDN9, CLDN10, CLDN11, CLDN12, CLDN13, CLDN14, CLDN15, CLDN16, CLDN17, CLDN20, CLDN23, Connexin 25 (Cx25), Cx26, Cx30, Cx30.3, Cx31, Cx31.1, Cx32, Cx36, C37, Cx43, Cx45, Cx46, Cx50, Nectin-4, Nectin-3, Nectin-2, Nectin-1, Necl-5, Necl-2, Necl-1, Necl-3, Necl-4, CD44, CD44v6, ICAM-1 (CD54), ICAM-2 (CD102), ICAM-3 (CD50), VCAM-1, NCAM, L1CAM, EpCAM, ALCAM (CD66), MCAM, MAdCAM-1, CEACAM5, CEACAM1, CEACAM6, CEACAM4, CEACAM7, CEACAM19, CEACAM3, L-selectin, E-selectin, P-selectin, integrin α5β1, αvβ1, αvβ3, αvβ5, αvβ6, αvβ8, fibronectin extradomain-β, CTGF, Siglec ligands (sialoglycan), MUC1, MUC1-Tn, MUC5AC, MUC15, MUC16, MUC17, EphA1, EphA2, EphA3, EphA4, EphA5, EphA6, EphA7, EphA8, EphB1, EphB2, EphB3, EphB4, MSLN, CSPG4, CA-IX, DLL3, DLL-4, GPC-3, FRα, PSMA, PSCA, GPNMB, HER3, ROR1, ROR2, Axl, TROP2, EGFRvIII, PTK7, GRP78, IGF-1R, PD-L2, TAG-72, NaPi2b, GUCY2C, LY6G6D, PDGFR-α, c-Met, 5T4, AGS-16, ALK1, ANG-2, SSTR2, ENPP3, gpA33, SLC44A4, SLC34A2, SLC39A6, Notch 1, Notch 2, Notch 3, Notch 4, STEAP1, CCR4, CXCR4, CD71, CD166, DKK-1, LRRC15, FAP, Tenascin, GP75, CD175, CD175s, CD176, Lewis Y, SLeA, MST1R, tissue factor, carbonio anhydrase 6, guanylate cyclase-C, ETBR, LIV-1, RANKL, STRA6, UPK1B, VTCN1, LY6E, Binin-4, Tweak, TweakR, SL TRK6, TEM1, TCRα/β, TCRγ/δ, CD3, 4-1BB, CD28, ICOS, CD27, OX40, GITR, LIGHT, CD226, NKG2D, NKG2C, NKp30, NKp44, NKp46, NKp80, DNAM-1, 2B4, KIR2DS, KIR3DS, NTBA, CD84, Ly9, PD-1, CTLA-4, TIGIT, LAG3, CD73, CD39, TIM3, VISTA, CD96, B7-H3, TMIGD2, BTLA, CD160, CD200R, CCR8, CEACAM1, NKG2A, NKG2B, KIR2DL, KIR3DL, LILRB1, LAIR1, LAIR2, CD16, CD32, CD64, CD89, CD35, CR2, CR3, CR4, CRIg, CD40, CSF1R, SIPRa, PSGL-1, VSIG4, CLEVER-1, LILRA1, LILRA2, LILRA3, LILRA4, LILRA5, LILRA6, LILRB1, LILRB2, LILRB3, LILRB4, LILRB5, LAIR1, LAIR2, CD200R, TREM1, TREM2, CD91, SR-A (CD204), CD36, MARCO, SCARF-1, TIM1, TIM-4, BAIL, Stabilin-2, RAGE, CD300f, CD14, CD44, MerTK, Tyro3, Axl, chemokine receptors, cytokine receptors, C-type lectin receptors, lipoprotein receptors, Siglecs, Toll-like receptors, DOTA (tetraxetan). DTPA (diethylenetriaminepentaacetic acid), and HSG (histamine-succinyl-glycine). In certain embodiments, the chemokine receptor is selected from the group consisting of CCR1, CCR2, CCR5, CCR7, CX3CR1, CXCR3, CXCR4, CXCR5 and CXCR6. In certain embodiments, the cytokine receptor is selected from the group consisting of IL-2R, IL-15R, IL-7R, TNFα, IL-12R, IFNα, IFNB and IFNγ. In certain embodiments, the C-type lectin receptor is selected from the group consisting of CD205, CD206, CD280, PLA2R, Dectin-1, Dectin-2, Dectin-3 (MCL), CLEC5A, Mincle, DC-SIGN, DC-SIGNR, DNGR-1 (CLEC9A), CLEC2, DCL-1 (CLEC13A), CLEC12B (MAH), MelLec (CLEC1A), LSECtin, LOX-1, BDCA-2, MGL (CLEC10A), CD93, Langerin, M1CL (CLEC12A) and DCIR. In certain embodiments, the lipoprotein receptor is selected from the group consisting of CD91, LOX-1 and LDLR. In certain embodiments, the Toll-like receptor (TLR) is selected from the group consisting of TLR1, TLR2, TLR4, TLR5 and TLR6. In certain embodiments, the Siglecs is selected from the group consisting of Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15 and Siglec-16.

In some embodiments, the non-CD47 binding Fab domain of the antibody fusion protein or Fab fusion protein, for example as configured in an exemplary format described in FV-48 to 78 and FV-128 to 190 above, binds to an antigen selected from the group consisting of CD38, SLAMF7, BCMA, GPRC5D, FcRH5, CD138, CD56, CD74, CD26, CD46, CD19, CD20, CD22, CD30, CD33, CD37, CD70, CD117, CD79b, CD123, CD52, CD98, CD205, PD-L1, HER2, EGFR, VEGFR2, VEGFR1, VEGF, CD93, TGFβ, IL-6, IL-6R, IL-8, GDF-15, GFRAL, FGFR2, FGFR3, FGFR4, FGFR1, GD2, GD3, B7-H3, B7-H4, phosphatidylserine, DR5, DR4, DR3, TL1A, CD95 (Fas), TNFR1, TNFR2, CD24, CD31, CD61, CD200, SLAMF3, SLAMF4, HLA-G, HLA-E, HLA-DR, NKG2D ligands (MICA, MICB, ULBP1-6), CD1d-presented lipid antigen, P-Cadherin, N-Cadherin, E-Cadherin, VE-Cadherin, R-cadherin (CDH4), K-cadherin (CDH6), VE-cadherin (CDH5), H-cadherin (CDH13), OB-cadherin, LI-cadherin (CDH17), CDH11, CDH19, CLDN18.2, CLDN6, CLDN3, CLDN4, CLDN7, CLDN1, CLDN2, CLDN5, CLDN8, CLDN9, CLDN1O, CLDN11, CLDN12, CLDN13, CLDN14, CLDN15, CLDN16, CLDN17, CLDN20, CLDN23, Connexin 25 (Cx25), Cx26, Cx30, Cx30.3, Cx31, Cx31.1, Cx32, Cx36, C37, Cx43, Cx45, Cx46, Cx50, Nectin-4, Nectin-3, Nectin-2, Nectin-1, Necl-5, Necl-2, Necl-1, Necl-3, Necl-4, CD44, CD44v6, ICAM-1 (CD54), ICAM-2 (CD102), ICAM-3 (CD50), VCAM-1, NCAM, L1CAM, EpCAM, ALCAM (CD66), MCAM, MAdCAM-1, CEACAM5, CEACAM1, CEACAM6, CEACAM4, CEACAM7, CEACAM19, CEACAM3, L-selectin, E-selectin, P-selectin, integrin α5β1, αvβ1, αvβ3, αvβ5, αvβ6, αvβ8, fibronectin extradomain-β, CTGF, Siglec ligands (sialoglycan), MUC1, MUC1-Tn, MUC5AC, MUC15, MUC16, MUC17, EphA1, EphA2, EphA3, EphA4, EphA5, EphA6, EphA7, EphA8, EphB1, EphB2, EphB3, EphB4, MSLN, CSPG4, CA-IX, DLL3, DLL-4, GPC-3, FRα, PSMA, PSCA, GPNMB, HER3, ROR1, ROR2, Axl, TROP2, EGFRvIII, PTK7, GRP78, IGF-1R, PD-L2, TAG-72, NaPi2b, GUCY2C, LY6G6D, PDGFR-α, c-Met, 5T4, AGS-16, ALK1, ANG-2, SSTR2, ENPP3, gpA33, SLC44A4, SLC34A2, SLC39A6, Notch 1, Notch 2, Notch 3, Notch 4, STEAP1, CCR4, CXCR4, CD71, CD166, DKK-1, LRRC15, FAP, Tenascin, GP75, CD175, CD175s, CD176, Lewis Y, SLeA, MST1R, tissue factor, carbonic anhydrase 6, GUCY2C, ETBR, LIV-1, RANKL, STRA6, UPK1B, VTCN1, LY6E, Binin-4, Tweak, TweakR, SL TRK6, and TEM1.

In certain embodiments, the non-CD47 binding Fab domain of the antibody fusion protein or Fab fusion protein, for example as configured in an exemplary format described in FV-48 to 78 and FV-128 to 190 above, comprise a set of VH and/or VL CDR sequences that are at least 70%, 75%, 80%, 85%, 90%, or 95% identical to the set of VH and/or VL CDR sequences according to the IMGT numbering scheme (The Immunologist, 7, 132-136 (1999); Dev Comp Immunol. 2003 January; 27 (1): 55-77) from an antibody selected from the group of antibodies as listed in Table 2 to 3.

In some embodiments, the non-CD47 binding Fab domain of the antibody fusion protein or Fab fusion protein, for example as configured in an exemplary format described in FV-48 to 78 and FV-128 to 190 above, binds to an antigen associated with a diseased cell or tissue, selected from the group consisting of PD-L1, CD38, SLAMF7, CD20, CD19, CD30, CD70, CD117, CA-IX, HER2, EGFR, VEGFR2, VEGFR1, VEGF, CD93, FGFR2, GD2, Claudin 18.2, Claudin 6, Claudin 1, Claudin 2, Claudin 3, Claudin 4, Claudin 7, B7-H3, DLL3, DR5, DR4, CD95, Phosphatidylserine, Nectin-4, CDH3, CDH6, CDH17, CDH2, integrins, CD44, ICAM-1, EpCAM, CEACAM5, CEACAM1, CEACAM6, CD24, HLA-G, FAP, CTGF and TL1A. In certain embodiments, the non-CD47 binding Fab domain of the antibody fusion protein or Fab fusion protein, for example as configured in an exemplary format described in FV-48 to 78 and FV-128 to 190 above, comprise a set of VH and/or VL CDR sequences that are at least 70%, 75%, 80%, 85%, 90%, or 95% identical to the set of VH and/or VL CDR sequences according to the IMGT numbering scheme from an antibody selected from the group of antibodies comprising anti-PD-L1 antibody BMS-936559, atezolizumab, durvalumab, avelumab, cosibelimab, sugemalimab, geptanolimab and envafolimab, anti-CD38 antibody daratumumab, isatuximab, SAR442085, felzartamab, mezagitamab, TAK-169, CID-103 and Y150, anti-SLAMF7 antibody elotuzumab and azintuxizumab, anti-CD20 antibody rituximab, ofatumumab, ocrelizumab, ublituximab, and obinutuzumab, anti-CD70 antibody cusatuzumab (ARGX-110), MDX1411, SEA-CD70, vorsetuzumab, IMM40H, IMM40M, anti-CD70 nanobody No. 1, 2 and 3 to 24 (WO2022262100), anti-CD70 nanobody No. 1 to 14 (CN113292652A), anti-CD70 antibodies (U.S. Pat. No. 11,377,500B2), LD70, BR108, MP-0533, and the anti-CD70 antibody moiety of SGN-75, SGN-CD70A, BMS-936561 (MDX-1203), AMG172, ARX-305, PRO-1160, CTX130, ALLO-316, P-CD70-ALLO1, 4SCAR70, C-4-29 and CAT-248, anti-CA-IX antibody girentuximab and BAY 79-4620, anti-HER2 antibody trastuzumab, pertuzumab, and margetuximab, anti-EGFR antibody cetuximab, panitumumab, necitumumab and nimotuzumab, anti-VEGFR2 antibody ramucirumab, alacizumab, olinvacimab, pulocimab and vulinacimab, anti-VEGFR1 antibody icrucumab, anti-VEGF antibody tarcocimab, varisacumab, brolucizumab, abicipar, IMC-IC11, faricimab, vanucizumab, dipacimab, navicixizumab, ivonescimab, anti-FGFR2 antibody bemarituzumab and aprutumab, anti-GD2 antibody dinutuximab and naxitamab, anti-DLL3 antibody rovalpituzumab, tarlatamab and PT217, anti-B7-H3 antibody ifinatamab, mirzotamab and enoblituzumab, anti-claudin 18.2 antibody zolbetuximab, gresonitamab, osemitamab, AB011, PT886 and TJ-CD4B, anti-claudin 6 antibody IMAB027, and TJ-C64B, anti-claudin 4 antibody KM3900, huKM3900, KM3934, 4D3, 5A5, 5D12 and KM3907, anti-claudin 3 antibody ABN501, MORAb-075, KMK3935, IgGH6, h4G3, 5A5 and KM3907, anti-claudin 2 antibody 1A2, anti-claudin 1 antibody 3A2, 7A5, 6F6, OM-7D3-B3 and humanized OM-7D3-B3, anti-CEACAM5 antibody hPR1A3, labetuzumab, cibisatamab, cergutuzumab, tusamitamab, AMG-211, BDC-2034, Clone 5G2, MN-3, MN-15, NEO-201, and 15-1-32, anti-CDH3 antibody PF-03732010, PF-06671008, FF-21101, TSP7 and TSP-S77R (Sci Rep. 2017 Jan. 3; 7:39518), anti-CDH6 antibody DS-6000 and NOV0712, anti-CDH17 antibody ARB202 and BI-905711, anti-integrin antibody volociximab (α581), etaracizumab (αvβ3), abciximab and intetumumab, anti-CD44 antibody RG7356, anti-ICAM-1 antibody bersanlimab, enlimomab and VBI-002, anti-EpCAM antibody adecatumumab, edrecolomab, citatuzumab, oportuzumab, solitomab, tucotuzumab and VBI-003, anti-Nectin-4 antibody enfortumab, BA3361, SBT6290 and ETx-22, anti-CD24 antibody hG7-BM3 (CN107226866A), humanized SWAll (U.S. Pat. No. 8,614,301B2) and ATG-031, anti-HLA-G antibody TTX-080, IVS-4001 and JNJ-78306358, anti-DR5 antibody conatumumab, drozitumab, lexatumumab, tigatuzumab, tilogotamab, benufutamab, zaptuzumab, INBRX-109, and IGM-8444, anti-phosphatidylserine antibody bavituximab, anti-FAP antibody sibrotuzumab, simlukafusp alfa, OS4 and MFP5, anti-CTGF antibody pamrevlumab (FG-3019), and anti-TL1A antibody tulisokibart (PRA023), PF-06480605 and TEV-48574. In certain embodiments, the non-CD47 binding Fab domain of the antibody fusion protein or Fab fusion protein, for example as configured in an exemplary format described in FV-48 to 78 and FV-128 to 190 above, binds to PD-L1 and comprises a pair of VH/VL amino acid sequences of SEQ ID NO: 144/145, or binds to CD38 and comprises a pair of VH/VL amino acid sequences of SEQ ID NO: 146/147, or binds to SLAMF7 and comprises a pair of VH/VL sequence of SEQ ID NO: 148/149, or binds to CD20 and comprises a pair of VH/VL sequence of SEQ ID NO: 150/151, or binds to HER2 and comprises a pair of VH/VL sequence of SEQ ID NO: 152/153, or binds to EGFR and comprises a pair of VH/VL amino acid sequences of SEQ ID NO: 154/155, or binds to FGFR2 and comprises a pair of VH/VL amino acid sequences of SEQ ID NO: 156/157, or binds to GD2 and comprises a pair of VH/VL sequence of SEQ ID NO: 158/159, or binds to claudin 18.2 and comprises a pair of VH/VL amino acid sequences of SEQ ID NO: 160/161, or binds to claudin 6 and comprises a pair of VH/VL sequence of SEQ ID NO: 162/163, or binds to CDH3 and comprises a pair of VH/VL amino acid sequences of SEQ ID NO: 164/165 or 166/167, or binds to CD44 and comprises a pair of VH/VL amino acid sequences of SEQ ID NO: 170/171, or binds to Nectin-4 and comprises a pair of VH/VL amino acid sequences of SEQ ID NO: 172/173, or binds to ICAM-1 and comprises a pair of VH/VL sequence of SEQ ID NO: 174/175, or binds to EpCAM and comprises a pair of VH/VL sequence of SEQ ID NO: 176/177, or binds to HLA-G and comprises a pair of VH/VL sequence of SEQ ID NO: 178/179, or binds to CD24 and comprises a pair of VH/VL sequence of SEQ ID NO: 180/181, or binds to DR5 and comprises a pair of VH/VL sequence of SEQ ID NO: 182/183, or binds to CD70 and comprises a pair of VH/VL sequence of SEQ ID NO: 184/185. In certain embodiments, the non-CD47 binding Fab domain of the antibody fusion protein or Fab fusion protein, for example as configured in an exemplary format described in FV-48 to 78 and FV-128 to 190 above, binds to CTGF or TL1A and comprises a pair of VH/VL amino acid sequences of SEQ ID NO: 186/187 or 188/189 respectively, wherein the antibody fusion protein or Fab fusion protein is used to treat a fibrotic disease such as idiopathic pulmonary fibrosis, hepatic fibrosis in NASH, scleroderma or systematic sclerosis.

In some embodiments, the non-CD47 binding Fab domain of the antibody fusion protein or Fab fusion protein, for example as configured in an exemplary format described in FV-29a to 47b, 48 to 78, and 128 to 190 above, binds to an antigen selected from the group consisting of TCRα/β, TCRγ/δ, CD3, 4-1BB, CD28, ICOS, CD27, CD70, OX40, GITR, LIGHT, CD226, NKG2D, NKG2C, NKp30, NKp44, NKp46, NKp80, DNAM-1, 2B4, KIR2DS, KIR3DS, NTBA, CD84, Ly9, PD-1, CTLA-4, TIGIT, LAG3, CD73, CD39, TIM3, VISTA, CD96, B7-H3, TMIGD2, BTLA, CD160, CD200R, CCR8, CEACAM1, NKG2A, NKG2B, KIR2DL, KIR3DL, LILRB1, LAIR1, LAIR2, CD16, CD32, CD64, CD89, CD35, CR2, CR3, CR4, CRIg, CD40, CSF1R, SIPRa, PSGL-1, VSIG4, CLEVER-1, LILRA1, LILRA2, LILRA3, LILRA4, LILRA5, LILRA6, LILRB1, LILRB2, LILRB3, LILRB4, LILRB5, LAIR1, LAIR2, CD200R, TREM1, TREM2, CD91, SR-A (CD204), CD36, MARCO, SCARF-1, TIM1, TIM-4, BAI1, Stabilin-2, RAGE, CD300f, CD14, CD44, MerTK, Tyro3, Axl, chemokine receptors, cytokine receptors, C-type lectin receptors, lipoprotein receptors, Siglecs, Toll-like receptors (TLR), DOTA (tetraxetan), DTPA (diethylenetriaminepentaacetic acid), and HSG (histamine-succinyl-glycine). In certain embodiments, the chemokine receptor is selected from the group consisting of CCR1, CCR2, CCR5, CCR7, CX3CR1, CXCR3, CXCR4, CXCR5 and CXCR6. In certain embodiments, the cytokine receptor is selected from the group consisting of IL-2R, IL-15R, IL-7R, TNFα, IL-12R, IFNα, IFNβ and IFNγ. In certain embodiments, the C-type lectin receptor is selected from the group consisting of CD205, CD206, CD280, PLA2R, Dectin-1, Dectin-2, Dectin-3 (MCL/CLEC5F8/CLEC4D), CLEC5A, Mincle, DC-SIGN, DC-SIGNR, DNGR-1 (CLEC9A), CLEC2, DCL-1 (CLEC13A), CLEC12B (MAH), MelLec (CLEC1A), LSECtin, LOX-1, BDCA-2, MGL (CLEC10A), CD93, Langerin, M1CL (CLEC12A) and DCIR. In certain embodiments, the lipoprotein receptor is selected from the group consisting of CD91, LOX-1 and LDLR. In certain embodiments, the Toll-like receptor (TLR) is selected from the group consisting of TLR1, TLR2, TLR4, TLR5 and TLR6. In certain embodiments, the Siglecs is selected from the group consisting of Siglec-1, Siglec-2, Siglec-3, Siglec-4, Siglec-S, Siglec-6, Siglec-7, Siglec-8, Siglec-9, Siglec-10, Siglec-11, Siglec-12, Siglec-13, Siglec-14, Siglec-15 and Siglec-16. In certain embodiments, the non-CD47 binding Fab domain of the antibody fusion protein or Fab fusion protein, for example as configured in an exemplary format described in FV-29a to 47b, 48 to 78, and 128 to 190 above, comprise a set of VH and/or VL CDR sequences that are at least 70%, 75%, 80%, 85%, 90%, or 95% identical to the set of VH and/or VL CDR sequences according to the IMGT numbering scheme (The Immunologist, 7, 132-136 (1999); Dev Comp Immunol, 2003 January; 27 (1): 55-77) from an antibody selected from the group of antibodies as listed in Table 4.

In certain embodiments, the non-CD47 binding Fab domain of the antibody fusion protein or Fab fusion protein, for example as configured in an exemplary format described in FV-29a to 47b, 48 to 78, and 128 to 190 above, binds to a surface antigen of an effector cell, selected from the group consisting of Dectin-1, Dectin-2, CLEC5A, MerTK, TREM1, MARCO, CLEVER-1, PSGL-1, VSIG4, CD40, CD205, CD206, CD36, CD91, DC-SIGN, CLEC9A, TLR5, LILRB1 (ILT2), LILRB2 (ILT4), LILRB4 (ILT3), NKG2D, NKp30, NKp46, NKp80, DNAM-1, PD-1, CTLA-4, TIGIT, LAG3, CD3, 4-1BB, OX40, ICOS, CD27 and CD70. In certain embodiments, the non-CD47 binding Fab domain of the antibody fusion protein or Fab fusion protein, for example as configured in an exemplary format described in FV-29a to 47b, 48 to 78, and 128 to 190 above, comprise a set of VH and/or VL CDR sequences that are at least 70%, 75%, 80%, 85%, 90%, or 95% identical to the set of VH and/or VL CDR sequences according to the IMGT numbering scheme from an antibody selected from the group consisting of anti-Dectin 1 antibody 2M24 (WO2022077006A1) and 15E2 (WO2008118587A3), anti-Dectin 2 antibody BDC-3042, anti-CLEC5A antibody IF7 (MAbs. 2022 January-December; 14 (1): 2040083), anti-MerTK antibody 18G7 (WO2020214995A1) and RGX-019, anti-CD205 antibody 3G9 (WO2009061996) and OBT076, anti-CD206 antibody CDX-1307, anti-DC-SIGN antibody hDIVI (U.S. Pat. No. 8,067,167B2), anti-TREM1 antibody PY159, anti-CLEVER1 antibody bexmarilimab, anti-PSGL-1 antibody VTX-0811, neihulizumab, and leiolizumab, anti-CD36 antibody ONA-0-v1 (WO2021176424A1), anti-LILRB1 antibody BND-22, NGM707, AGEN1571, ATG-032 and DM002, anti-LILRB2 antibody MK-4830, JTX-8064, NGM707, IO-108, ES009, ATG-032 and DM002, anti-LILRB4 antibody IO-202, MK-0482, BND-35, NGM831, JTX1484 and SG2901, anti-NKG2D antibody A49 and A44 (WO2021041878A1) and KYK-2.0 (US20110150870A1), anti-CD3 antibody SP34 (J Immunol. 1986 Aug. 15; 137 (4): 1097-100), UCHT1 (Pharmacol Ther. 2018 February; 182:161-175), mosunetuzumab, tarlatamab, PF-06671008, tebentafusp and TNB-383B, anti-4-1BB antibody utomilumab, AGEN2373, LVGN6051, GEN1046 and TJ-C64B, anti-PD-1 antibody nivolumab and pembrolizumab, anti-CTLA-4 antibody ipilimumab, tremelimumab, botensilimab, nurulimab and BA-3017, and anti-TIGIT antibody tiragolumab, vibostolimab, etigilimab, BGB-A1217 and EOS-448. In certain embodiments, the non-CD47 binding Fab domain of the antibody fusion protein or Fab fusion protein, for example as configured in an exemplary format described in FV-29a to 47b, 48 to 78, and 128 to 190 above, binds to CD205 and comprises a pair of VH/VL amino acid sequences of SEQ ID NO: 190/191, or binds to Dectin-1 and comprises a pair of VH/VL amino acid sequences of SEQ ID NO: 192/193 or 194/195, or binds to MerTK and comprises a pair of VH/VL amino acid sequences of SEQ ID NO: 196/197, or binds to CD206 and comprises a pair of VH/VL amino acid sequences of SEQ ID NO: 198/199, or binds to DC-SIGN and comprises a pair of VH/VL amino acid sequences of SEQ ID NO: 200/201, or binds to TREM1 and comprises a pair of VH/VL amino acid sequences of SEQ ID NO: 202/203, or binds to NKG2D and comprises a pair of VH/VL amino acid sequences of SEQ ID NO: 204/205, or binds to CD3 and comprises a pair of VH/VL amino acid sequences of SEQ ID NO: 206/207, or binds to 4-1BB and comprises a pair of VH/VL amino acid sequences of SEQ ID NO: 208/209, or binds to ILT2 and comprises a pair of VH/VL amino acid sequences of SEQ ID NO: 210/211, or binds to CTLA4 and comprises a pair of VH/VL sequences of SEQ ID NO: 212/213.

In certain embodiments, the non-CD47 binding Fab domain of the antibody fusion protein or Fab fusion protein, for example as configured in an exemplary format described in FV-29a to 47b, 48 to 78, and 128 to 190 above, binds to a hapten antigen selected from the group consisting of hapten peptide histamine-succinyl-glycine (HSG) and chelator DOTA, DOTATATE, DOTA-Bn, DO2A, DTPA, DATA, PCTA, NOTA, NOTP, TRAP, TACN, AAZTA, H4pypa, H2dedpa, H2CHXhox, H2hox, HBED and DFO, as described in (Sneddon et al., Curr Opin Chem Biol, 2021). In certain embodiments, the non-CD47 binding Fab domain of the antibody fusion protein or Fab fusion protein, for example as configured in an exemplary format described in FV-29a to 47b, 48 to 78, and 128 to 190 above, comprise a set of VH and/or VL CDR sequences that are at least 70%, 75%, 80%, 85%, 90%, or 95% identical to the set of VH and/or VL CDR sequences according to the IMGT numbering scheme from an antibody selected from the group consisting of anti-DOTA antibody 2D12.5 (Corneillie et al., J Am Chem Soc, 2003), C825 (U.S. Pat. No. 8,648,176), huC825 (US20200140543A1) and hLL2 (US20030124057A1), anti-DTPA antibody Mab 734 (US20030162709A1), and anti-HSG antibody Mab 679 (US20030162709A1).

In some embodiments, the non-CD47 binding Fab domain and the single-chain third antigen-binding domain of the antibody fusion protein or Fab fusion protein, for example as configured in an exemplary format described in FV-43a to 46b, and 136 to 190 above, or the two non-CD47 binding Fab domains of the antibody fusion protein, as configured in exemplary format FV-128 to 135 above, bind to two different antigens selected from the group consisting of CD38, SLAMF7, BCMA, GPRC5D, FcRH5, CD138, CD56, CD74, CD26, CD46, CD19, CD20, CD22, CD30, CD33, CD37, CD70, CD117, CD79b, CD123, CD52, CD98, CD205, PD-L1, HER2, EGFR, VEGFR2, VEGFR1, VEGF, CD93, TGFβ, IL-6, IL-6R, IL-8, GDF-15, GFRAL, FGFR2, FGFR3, FGFR4, FGFR1, GD2, GD3, B7-H3, B7-H4, phosphatidylserine, DR5, DR4, DR3, TL1A, CD95 (Fas), TNFR1, TNFR2, CD24, CD31, CD61, CD200, SLAMF3, SLAMF4, HLA-G, HLA-E, HLA-DR, NKG2D ligands (MICA, MICB, ULBP1-6), CD1d-presented lipid antigen, P-Cadherin, N-Cadherin, E-Cadherin, VE-Cadherin, R-cadherin (CDH4), K-cadherin (CDH6), VE-cadherin (CDH5), H-cadherin (CDH13), OB-cadherin, LI-cadherin (CDH17), CDH11, CDH19, CLDN18.2, CLDN6, CLDN3, CLDN4, CLDN7, CLDN1, CLDN2, CLDN5, CLDN8, CLDN9, CLDN1O, CLDN11, CLDN12, CLDN13, CLDN14, CLDN15, CLDN16, CLDN17, CLDN20, CLDN23, Connexin 25 (Cx25), Cx26, Cx30, Cx30.3, Cx31, Cx31.1, Cx32, Cx36, C37, Cx43, Cx45, Cx46, Cx50, Nectin-4, Nectin-3, Nectin-2, Nectin-1, Necl-5, Necl-2, Necl-1, Necl-3, Necl-4, CD44, CD44v6, ICAM-1 (CD54), ICAM-2 (CD102), ICAM-3 (CD50), VCAM-1, NCAM, L1CAM, EpCAM, ALCAM (CD66), MCAM, MAdCAM-1, CEACAM5, CEACAM1, CEACAM6, CEACAM4, CEACAM7, CEACAM19, CEACAM3, L-selectin, E-selectin, P-selectin, integrin α5B1, αvβ1, αvβ3, αvβ5, αvβ6, αvβ8, fibronectin extradomain-β, CTGF, Siglec ligands (sialoglycan), MUC1, MUC1-Tn, MUC5AC, MUC15, MUC16, MUC17, EphA1, EphA2, EphA3, EphA4, EphA5, EphA6, EphA7, EphA8, EphB1, EphB2, EphB3, EphB4, MSLN, CSPG4, CA-IX, DLL3, DLL-4, GPC-3, FRα, PSMA, PSCA, GPNMB, HER3, ROR1, ROR2, Axl, TROP2, EGFRvIII, PTK7, GRP78, IGF-1R, PD-L2, TAG-72, NaPi2b, GUCY2C, LY6G6D, PDGFR-α, c-Met, 5T4, AGS-16, ALK1, ANG-2, SSTR2, ENPP3, gpA33, SLC44A4, SLC34A2, SLC39A6, Notch 1, Notch 2, Notch 3, Notch 4, STEAP1, CCR4, CXCR4, CD71, CD166, DKK-1, LRRC15, FAP, Tenascin, GP75, CD175, CD175s, CD176, Lewis Y, SLeA, MST1R, tissue factor, carbonic anhydrase 6, guanylate cyclase-C, ETBR, LIV-1, RANKL, STRA6, UPK1B, VTCN1, LY6E, Binin-4, Tweak, TweakR, SLTRK6, TEM1, TCRα/β, TCRγ/δ, CD3, 4-1BB, CD28, ICOS, CD27, OX40, GITR, LIGHT, CD226, NKG2D, NKG2C, NKp30, NKp44, NKp46, NKp80, DNAM-1, 2B4, KIR2DS, KIR3DS, NTBA, CD84, Ly9, PD-1, CTLA-4, TIGIT, LAG3, CD73, CD39, TIM3, VISTA, CD96, B7-H3, TMIGD2, BTLA, CD160, CD200R, CCR8, CEACAM1, NKG2A, NKG2B, KIR2DL, KIR3DL, LILRB1, LAIR1, LAIR2, CD16, CD32, CD64, CD89, CD35, CR2, CR3, CR4, CRIg, CD40, CSF1R, SIPRa, PSGL-1, VSIG4, CLEVER-1, LILRA1, LILRA2, LILRA3, LILRA4, LILRA5, LILRA6, LILRB1, LILRB2, LILRB3, LILRB4, LILRB5, LAIR1, LAIR2, CD200R, TREM1, TREM2, CD91, SR-A (CD204), CD36, MARCO, SCARF-1, TIM1, TIM-4, BAI1, Stabilin-2, RAGE, CD300f, CD14, CD44, MerTK, Tyro3, Axl, chemokine receptors, cytokine receptors, C-type lectin receptors, lipoprotein receptors, Siglecs, Toll-like receptors (TLR), DOTA (tetraxetan), DTPA (diethylenetriaminepentaacetic acid), and HSG (histamine-succinyl-glycine). In certain embodiments, the chemokine receptor is selected from the group consisting of CCR1, CCR2, CCR5, CCR7, CX3CR1, CXCR3, CXCR4, CXCR5 and CXCR6. In certain embodiments, the cytokine receptor is selected from the group consisting of IL-2R, IL-15R, IL-7R, TNFα, IL-12R, IFNα, IFNβ and IFNγ. In certain embodiments, the C-type lectin receptor is selected from the group consisting of CD205, CD206, CD280, PLA2R, Dectin-1, Dectin-2, Dectin-3 (MCL), CLEC5A, Mincle, DC-SIGN, DC-SIGNR, DNGR-1 (CLEC9A), CLEC2, DCL-1 (CLEC13A), CLEC12B (MAH), MelLec (CLEC1A), LSECtin, LOX-1, BDCA-2, MGL (CLEC10A), CD93, Langerin, M1CL (CLEC12A) and DCIR. In certain embodiments, the lipoprotein receptor is selected from the group consisting of CD91, LOX-1 and LDLR. In certain embodiments, the Toll-like receptor (TLR) is selected from the group consisting of TLR1, TLR2, TLR4, TLR5 and TLR6. In certain embodiments, the Siglecs is selected from the group consisting of Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15 and Siglec-16.

In some embodiments, the single-chain third antigen-binding domain of the antibody fusion protein or Fab fusion protein, for example as configured in an exemplary format described in FV-43a to 46b, and 136 to 190 above, comprises a peptide or polypeptide with antigen binding, a ligand or fragment thereof with receptor binding, a receptor or fragment thereof with ligand binding, a cytokine or fragment thereof with receptor binding, a chemokine or fragment thereof with receptor binding, a single chain fragment variable (scFv), a VHH antibody or nanobody, a VH single chain antibody (VH dAb) or a VL single chain antibody (VL dAb), an immunoglobulin new antigen receptor (IgNAR), or other single-chain domain antibody, or an alternative single-chain scaffold known in the arts to function as antigen binding domain, such as anticalin, DARPIN, affibody, affimer, centryin domain, fibronectin domain, D-Domain, nanofitin, adnectin, knottin, kunitz, avimer, affilin, ADAPT, and the like. In some embodiments, the single-chain third antigen-binding domain of the antibody fusion protein or Fab fusion protein, for example as configured in an exemplary format described in FV-43a to 46b, and 136 to 190 above, comprises a peptide or polypeptide selected from the group of peptides and polypeptides as set forth in Table 5. In some embodiments, the single-chain third antigen-binding domain of the antibody fusion protein or Fab fusion protein, for example as configured in an exemplary format described in FV-43a to 46b, and 136 to 190 above, bind to an antigen selected from the group consisting of TGFβ, VEGF, CTGF, TL1A, GDF15, IL-8, IL-6, Dectin-1, CLEC5A, MerTK, CD205, CD206, CD91, ILT2, ILT4, TLR5, NKG2D, NKp46, NKp30, CD28, ICOS, NKG2D ligands, Siglec ligands (sialoglycan), CD70, CD24, HLA-G, cadherins, claudins, nectins, integrin, and FGFR. In certain embodiments, the single-chain third antigen-binding domain of the antibody fusion protein or Fab fusion protein, for example as configured in an exemplary format described in FV-43a to 46b, and 136 to 190 above, comprises an amino acid sequence selected from the group consisting of SEQ ID NO: 215 to 241. In certain embodiments, the single-chain third antigen-binding domain of the antibody fusion protein or Fab fusion protein, for example as configured in an exemplary format described in FV-43a to 46b, and 136 to 190 above, comprises a scFv consisting of VH-linker-VL or VL-linker-VH, wherein the linker comprises SEQ ID NO: 120 or 121, and the VH and VL comprises a pair of VH x VL sequence selected from the group comprising SEQ ID NO: 144x145, 146x147, 148x149, 150x151, 152x153, 154x155, 156x157, 158x159, 160x161, 162x163, 164x165, 166x167, 168x169, 170x171, 172x173, 174x175, 176x177, 178x179, 180x181, 182x183, 184x185, 186x187, 188x189, 190x191, 192x193, 194x195, 196x197, 198x199, 200x201, 202x203, 204x205, 206x207, 208x209, 210x211, 212x213. In certain embodiments, the single-chain third antigen-binding domain of the antibody fusion protein or Fab fusion protein, for example as configured in an exemplary format described in FV-43a to 46b, and 136 to 190 above, bind to TGFB. In certain embodiments, the single-chain third antigen-binding domain of the antibody fusion protein or Fab fusion protein, comprises an extracellular domain of TOFβRII or fragment thereof capable of binding to TGFB. In certain embodiments, the single-chain third antigen-binding domain of the antibody fusion protein or Fab fusion protein comprises an amino acid sequence of SEQ ID NO:217. In certain embodiments, the single-chain third antigen-binding domain of the antibody fusion protein or Fab fusion protein, for example as configured in an exemplary format described in FV-43a to 46b, and 136 to 190 above, comprises a peptide or polypeptide binding to CTGF. In certain embodiments, the single-chain third antigen-binding domain of the antibody fusion protein or Fab fusion protein, comprises the peptide BLR-100 or BLR-200 binding to CTGF, or a scFv derived from the anti-CTGF antibody pamrevlumab comprising VH/VL of SEQ ID NO: 186/187. In certain embodiments, the single-chain third antigen-binding domain of the antibody fusion protein or Fab fusion protein, for example as configured in an exemplary format described in FV-43a to 46b, and 136 to 190 above, bind to TL1A. In certain embodiments, the single-chain third antigen-binding domain of the antibody fusion protein or Fab fusion protein, comprises an extracellular domain of DR3 (death receptor 3) or fragment thereof capable of binding to TL1A, or a scFv derived from the anti-TL1A antibody of tulisokibart (PRA023) comprising a VH/VL pair of SEQ ID NO: 188/189, PF-06480605 or TEV-48574. In certain embodiments, the single-chain third antigen-binding domain of the antibody fusion protein or Fab fusion protein, comprises a wild type extracellular domain of DR3 (SEQ ID NO: 218) or its variants H3 (SEQ ID NO: 219), 06 (SEQ ID NO: 220), A7, 112, G6 or N8 (see {Levin, 2017 #840}). In certain embodiments, the single-chain third antigen-binding domain of the antibody fusion protein or Fab fusion protein binds to Dectin-1. In certain embodiments, the single-chain third antigen-binding domain of the antibody fusion protein or Fab fusion protein comprises a Dectin-1-binding scFv consisting of VH-linker-VL wherein the VH and VL comprise SEQ ID NO: 194 and 195 respectively or SEQ ID NO: 192 and 193 respectively, and the linker comprises SEQ ID NO: 121. In certain embodiments, the single-chain third antigen-binding domain of the antibody fusion protein or Fab fusion protein binds to IL T2 and/or IL T4. In certain embodiments, the single-chain third antigen-binding domain of the antibody fusion protein or Fab fusion protein comprises a ILT2/4-binding scFv consisting of VH-linker-VL or VL-linker-VH, wherein the VH and VL comprise SEQ ID NO: 210 and 211 respectively and the linker comprises SEQ ID NO: 121. In certain embodiments, the single-chain third antigen-binding domain of the antibody fusion protein or Fab fusion protein binds to NKG2D. In certain embodiments, the single-chain third antigen-binding domain of the antibody fusion protein or Fab fusion protein comprises a NKG2D-binding scFv consisting of VH-linker-VL or VL-linker-VH, wherein the VH and VL comprise SEQ ID NO: 204 and 205 respectively and the linker comprises SEQ ID NO: 121. In certain embodiments, the single-chain third antigen-binding domain of the antibody fusion protein or Fab fusion protein comprises a NKG2D ligand comprising an amino acid sequence selected from the group consisting of SEQ ID NO: 231 to 238. In certain embodiments, the single-chain third antigen-binding domain of the antibody fusion protein or Fab fusion protein binds to NKp46. In certain embodiments, the single-chain third antigen-binding domain of the antibody fusion protein or Fab fusion protein comprises a NKp46-binding scFv consisting of VH-linker-VL or VL-linker-VH, wherein the linker comprises SEQ ID NO: 121 and the VH and VL is of the VH and VL of anti-NKp46 antibody IPH6101. In certain embodiments, the single-chain third antigen-binding domain of the antibody fusion protein or Fab fusion protein binds to NKp30. In certain embodiments, the single-chain third antigen-binding domain of the antibody fusion protein or Fab fusion protein comprises a NKp30-binding scFv consisting of VH-linker-VL or VL-linker-VH, wherein the linker comprises SEQ ID NO: 121 and the VH and VL is of the VH and VL of anti-NKp30 antibody CTX-441 or CTX-8573. In certain embodiments, the antibody fusion protein or Fab fusion protein, for example as configured in an exemplary format described in FV-43a to 46b, and 136 to 190, wherein the single-chain third antigen-binding domain binds to TGFβ, VEGF, CTGF, TL1A, Dectin-1, ILT2, ILT4, NKG2D, NKp46 or NKp30, comprises one, two, three, four or more SIRP IgV monomers comprising a sequence selected from the group consisting of SEQ ID NO: 3 to 113 and is used to treat cancer or fibrotic diseases.

In some embodiments, the non-CD47 binding single-chain polypeptide domain of the Fe fusion protein, for example as configured in an exemplary format described in FV-101 to 104, and 109 to 113 above, binds to a soluble antigen, an aggregate of antigen, a surface antigen on a cell, a surface antigen on a microbe, or a hapten. In some embodiments, the non-CD47 binding single-chain polypeptide domain binds to a surface antigen on a diseased cell, an infected cell or an effector cell. In certain embodiments, the diseased cell is a cancer cell, a fibrotic cell, an atherosclerotic cell, an inflammatory cell or a senescent cell. In certain embodiments, the infected cell is a cell infected by bacterium, fungus, virus and/or parasite. In certain embodiments, the effector cell is a myeloid cell, a lymphocyte or a granulocyte. In certain embodiments, the effector cell is a macrophage, a monocyte, a dendritic cell, a B cell, a T cell, a NK cell, a NKT cell, an innate lymphoid cell (ILC), a neutrophil, and/or a mast cell. In certain embodiments, the non-CD47 binding single-chain polypeptide domain of the Fc fusion protein binds to a microbe, wherein the microbe is a bacterium, a fungus, a protozoa, or a virus. In certain embodiments, the non-CD47 binding single-chain polypeptide domain of the Fe fusion protein binds to a hapten, wherein the hapten comprises a chelator that binds to a radionuclide or radiometal. In certain embodiments, the radionuclide is selected from the group consisting of ²²⁵Ac, ²¹¹At, ²¹²Bi, ⁶²Cu, ⁶⁴Cu, ⁶⁷Cu, ⁶⁶Ga, ⁶⁷Ga, ⁶⁸Ga, ¹²⁵I, ¹³¹I, ¹¹¹In, ¹⁷⁷Lu, ²¹²Pb, ¹⁸⁶Re, ¹⁸⁸Re, ⁴⁴⁵c, ¹⁴⁹Th, ¹⁵²Tb, ¹⁵⁵Tb, ¹⁶¹Tb, ⁹⁹Y and ⁸⁹Zr. In certain embodiments, the chelator is a macrocyclic chelator, an acyclic chelator or a hybrid chelator. In additional embodiments, the chelator is selected from the group consisting of DOTA, DOTATATE, DOTA-Bn, DO2A, DTPA, DATA, PCTA, NOTA, NOTP, TRAP, 1,4,7-Triazacyclononane (TACN), AAZTA, H4pypa, H2dedpa, H2CHXhox, H2hox, HBED and DFO, as described in (Sneddon et al., Curr Opin Chem Biol, 2021). In certain embodiments, the hapten comprises a hapten peptide histamine-succinyl-glycine (HSG).

In some embodiments, the non-CD47 binding single-chain polypeptide domain of the Fe fusion protein, for example as configured in an exemplary format described in FV-101 to 104, and 109 to 113 above, binds to an antigen selected from the group consisting of CD38, SLAMF7, BCMA, GPRC5D, FcRH5, CD138, CD56, CD74, CD26, CD46, CD19, CD20, CD22, CD30, CD33, CD37, CD70, CD117, CD79b, CD123, CD52, CD98, CD205, PD-L1, HER2, EGFR, VEGFR2, VEGFR1, VEGF, CD93, TGFβ, IL-6, IL-6R, IL-8, GDF-15, GFRAL, FGFR2, FGFR3, FGFR4, FGFR1, GD2, GD3, B7-H3, B7-H4, phosphatidylserine, DR5, DR4, DR3, TL1A, CD95 (Fas), TNFR1, TNFR2, CD24, CD31, CD61, CD200, SLAMF3, SLAMF4, HLA-G, HLA-E, HLA-DR, NKG2D ligands (MICA, MICB, ULBP1-6), CD1d-presented lipid antigen, P-Cadherin, N-Cadherin, E-Cadherin, VE-Cadherin, R-cadherin (CDH4), K-cadherin (CDH6), VE-cadherin (CDH5), H-cadherin (CDH13), OB-cadherin, LI-cadherin (CDH17), CDH11, CDH19, CLDN18.2, CLDN6, CLDN3, CLDN4, CLDN7, CLDN1, CLDN2, CLDN5, CLDN8, CLDN9, CLDN10, CLDN11, CLDN12, CLDN13, CLDN14, CLDN15, CLDN16, CLDN17, CLDN20, CLDN23, Connexin 25 (Cx25), Cx26, Cx30, Cx30.3, Cx31, Cx31.1, Cx32, Cx36, C37, Cx43, Cx45, Cx46, Cx50, Nectin-4, Nectin-3, Nectin-2, Nectin-1, Necl-5, Necl-2, Necl-1, Necl-3, Necl-4, CD44, CD44v6, ICAM-1 (CD54), ICAM-2 (CD102), ICAM-3 (CD50), VCAM-1, NCAM, L1CAM, EpCAM, ALCAM (CD66), MCAM, MAdCAM-1, CEACAM5, CEACAM1, CEACAM6, CEACAM4, CEACAM7, CEACAM19, CEACAM3, L-selectin, E-selectin, P-selectin, integrin α5β1, αvβ1, αvβ3, αvβ5, αvβ6, αvβ8, fibronectin extradomain-β, CTGF, Siglec ligands (sialoglycan), MUC1, MUC1-Tn, MUC5AC, MUC15, MUC16, MUC17, EphA1, EphA2, EphA3, EphA4, EphA5, EphA6, EphA7, EphA8, EphB1, EphB2, EphB3, EphB4, MSLN, CSPG4, CA-IX, DLL3, DLL-4, GPC-3, FRα, PSMA, PSCA, GPNMB, HER3, ROR1, ROR2, Axl, TROP2, EGFRvIII, PTK7, GRP78, IGF-1R, PD-L2, TAG-72, NaPi2b, GUCY2C, LY6G6D, PDGFR-α, c-Met, 5T4, AGS-16, ALK1, ANG-2, SSTR2, ENPP3, gpA33, SLC44A4, SLC34A2, SLC39A6, Notch 1, Notch 2, Notch 3, Notch 4, STEAP1, CCR4, CXCR4, CD71, CD166, DKK-1, LRRC15, FAP, Tenascin, GP75, CD175, CD175s, CD176, Lewis Y, SLeA, MST1R, tissue factor, carbonic anhydrase 6, guanylate cyclase-C, ETBR, LIV-1, RANKL, STRA6, UPK1B, VTCN1, LY6E, Binin-4, Tweak, TweakR, SLTRK6, TEM1, TCRα/β, TCRγ/6, CD3, 4-1BB, CD28, ICOS, CD27, OX40, GITR, LIGHT, CD226, NKG2D, NKG2C, NKp30, NKp44, NKp46, NKp80, DNAM-1, 2B4, KIR2DS, KIR3DS, NTBA, CD84, Ly9, PD-1, CTLA-4, TIGIT, LAG3, CD73, CD39, TIM3, VISTA, CD96, B7-H3, TMIGD2, BTLA, CD160, CD200R, CCR8, CEACAM1, NKG2A, NKG2B, KIR2DL, KIR3DL, LILRB1, LAIR1, LAIR2, CD16, CD32, CD64, CD89, CD35, CR2, CR3, CR4, CRIg, CD40, CSF1R, SIPRa, PSGL-1, VSIG4, CLEVER-1, LILRA1, LILRA2, LILRA3, LILRA4, LILRA5, LILRA6, LILRB1, LILRB2, LILRB3, LILRB4, LILRB5, LAIR1, LAIR2, CD200R, TREM1, TREM2, CD91, SR-A (CD204), CD36, MARCO, SCARF-1, TIM1, TIM-4, BAI1, Stabilin-2, RAGE, CD300f, CD14, CD44, MerTK, Tyro3, Axl, chemokine receptors, cytokine receptors, C-type lectin receptors, lipoprotein receptors, Siglecs, Toll-like receptors (TLR), DOTA (tetraxetan), DTPA (diethylenetriaminepentaacetic acid), and HSG (histamine-succinyl-glycine). In certain embodiments, the chemokine receptor is selected from the group consisting of CCR1, CCR2, CCR5, CCR7, CX3CR1, CXCR3, CXCR4, CXCR5 and CXCR6. In certain embodiments, the cytokine receptor is selected from the group consisting of IL-2R, IL-15R, IL-7R, TNFα, IL-12R, IFNα, IFNβ and IFNγ. In certain embodiments, the C-type lectin receptor is selected from the group consisting of CD205, CD206, CD280, PLA2R, Dectin-1, Dectin-2, Dectin-3 (MCL/CLEC5F8/CLEC4D), CLEC5A, Mincle, DC-SIGN, DC-SIGNR, DNGR-1 (CLEC9A), CLEC2, DCL-1 (CLEC13A), CLEC12B (MAH), MelLec (CLEC1A), LSECtin, LOX-1, BDCA-2, MGL (CLEC10A), CD93, Langerin, M1CL (CLEC12A) and DCIR. In certain embodiments, the lipoprotein receptor is selected from the group consisting of CD91, LOX-1 and LDLR. In certain embodiments, the Toll-like receptor (TLR) is selected from the group consisting of TLR1, TLR2, TLR4, TLR5 and TLR6. In certain embodiments, the Siglecs is selected from the group consisting of Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15 and Siglec-16. In certain embodiments, the non-CD47 binding single-chain polypeptide domain of the Fe fusion protein, for example as configured in an exemplary format described in FV-101 to 104, and 109 to 113 above, comprises a peptide or polypeptide with antigen binding, a ligand or fragment thereof with receptor binding, a receptor or fragment thereof with ligand binding, a single chain fragment variable (scFv), a VHH antibody or nanobody, a VH single chain antibody (VH dAb) or a VL single chain antibody (VL dAb), an immunoglobulin new antigen receptor (IgNAR), or other single-chain domain antibody, or an alternative single-chain scaffold known in the arts to function as antigen binding domain, such as anticalin, DARPIN, affibody, affimer, centryin domain, fibronectin domain, D-Domain, nanofitin, adnectin, knottin, kunitz, avimer, affilin, ADAPT, and the like. In certain embodiments, the non-CD47 binding single-chain polypeptide domain of the Fc fusion protein, for example as configured in an exemplary format described in FV-101 to 104, and 109 to 113 above, comprises a peptide or polypeptide selected from the group of peptides and polypeptides as set forth in Table 5. In certain embodiments, the non-CD47 binding single-chain polypeptide domain of the Fe fusion protein, for example as configured in an exemplary format described in FV-101 to 104, and 109 to 113 above, binds to Dectin-1, CLEC5A, MerTK, CD205, CD206, CD91, ILT2, ILT4, TLR5, NKG2D, NKp46, NKp30, CD28, ICOS, NKG2D ligands, Siglec ligands (sialoglycan), CD70, CD24, HLA-G, cadherins, claudins, nectins, integrin, or FGFR. In certain embodiments, the non-CD47 binding single-chain polypeptide domain of the Fe fusion protein, comprises an amino acid sequence selected from the group consisting of SEQ ID NO: 221 to 241.

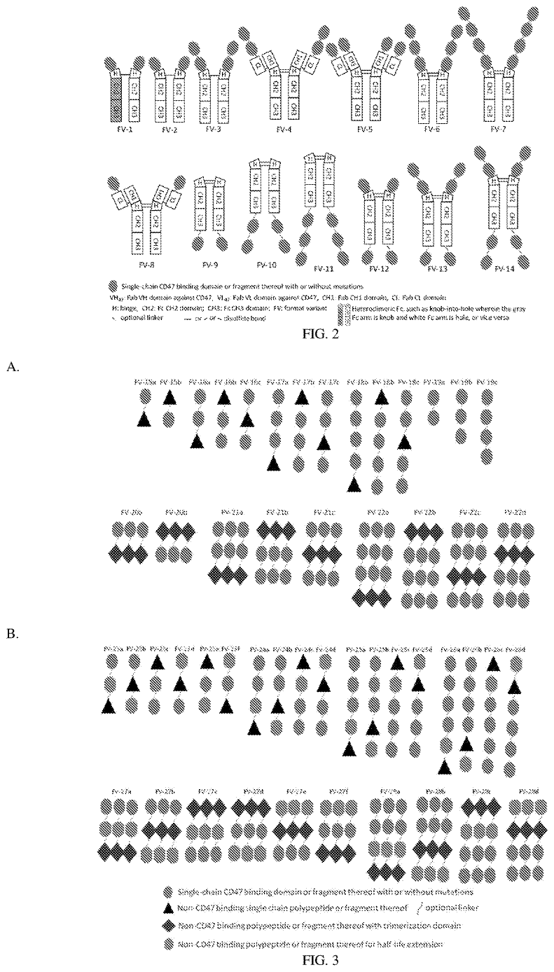

In some embodiments, a protein comprising a said fusion polypeptide comprising a SIRP IgV domain is configured in FV-114 as set forth in FIG. 8, wherein the fusion polypeptide from N-terminal to C-terminal comprises one CD47-binding SIRP IgV monomer directly linked to the N-terminal of a human IgG Fc chain comprising hinge-CH2-CH3 and the Fc chain at its C-terminal is further linked to a trimerization motif-comprising third domain preferentially through a linker, wherein two Fc chains form a homodimeric Fc and the two trimerization motifs of one homodimeric Fc trimerize with a trimerization motif of another homodimeric Fc. In certain embodiments, the SIRP IgV monomer comprises an amino acid sequence selected from the group consisting of SEQ ID NO: 3 to 81. In certain embodiment, the SIRP monomer comprises a mutation of K53H. In certain embodiment, the SIRP monomer comprises a mutation of R69H. In certain embodiment, the SIRP monomer comprises a mutation of Q52H. In certain embodiment, the SIRP monomer comprises a mutation of K68H. In certain embodiment, the SIRP monomer comprises a mutation of Q52H+K68H. In certain embodiments, the Fc chain comprises an amino acid sequence selected from the group consisting of SEQ ID NO: 130 to 135. In certain embodiments, the Fc chain comprises an amino acid sequence of SEQ ID NO: 135. In some embodiments, a protein comprising a said fusion polypeptide comprising a SIRP IgV domain is configured in a format selected from the group consisting of FV-20a to 22d as set forth in FIG. 3, wherein the fusion polypeptide comprising a SIRP IgV domain and a trimerization motif-comprising domain linked together optionally through a linker and the trimerization motif promotes trimerization of three such fusion polypeptides. In certain embodiments, the fusion polypeptide comprises one SIRPα, SIRPβ or SIRPγ IgV monomer and is configured in FV-20a. In certain embodiments, the fusion polypeptide comprises one SIRP IgV monomer comprising an amino acid sequence selected from the group consisting of SEQ ID NO: 3 to 81. In certain embodiment, the SIRP monomer comprises a mutation of K53H. In certain embodiment, the SIRP monomer comprises a mutation of R69H. In certain embodiment, the SIRP monomer comprises a mutation of Q52H. In certain embodiment, the SIRP monomer comprises a mutation of K68H. In certain embodiment, the SIRP monomer comprises a mutation of Q52H+K68H. In certain embodiments, the SIRP IgV domain of the fusion polypeptide comprises two or three SIRP IgV monomers, wherein the two or three SIRP IgV monomers comprise SIRPα, SIRPβ and/or SIRPγ IgV monomers. In certain embodiments, the fusion polypeptide comprises two or three SIRP IgV monomers, wherein the SIRP IgV monomers comprise the same or different amino acid sequences selected from the group consisting of SEQ ID NO: 3 to 81. In some embodiments, the fusion polypeptide comprising a SIRP IgV domain and a trimerization-motif-comprising domain additionally comprises a third antigen-binding domain, wherein the third antigen-binding domain is a single-chain polypeptide binding to a third antigen and is linked preferentially through a linker to the C-terminal of SIRP IgV domain or the trimerization-motif-comprising domain. In certain embodiments, the fusion polypeptide comprising the third antigen-binding domain is configured in a format selected from the group consisting of FV-27a to 28d as set forth in FIG. 3. In certain embodiments, the fusion polypeptide comprising the third antigen-binding domain comprises one SIRP IgV monomer and is configured in FV-27a or 27b as set forth in FIG. 3. In certain embodiments, the fusion polypeptide comprises a third antigen-binding domain, wherein the third antigen-binding domain comprises a single-chain domain that binds to human albumin (HSA). In certain embodiments, the HSA-binding third domain comprises an amino acid sequence of SEQ ID NO: 214.

In certain embodiments, the trimerization motif-comprising domain of the fusion polypeptide of FV-114 and FV-20a to 28d above comprises a polypeptide or fragment thereof a tumor necrosis factor (TNF) superfamily protein comprising a trimerization motif. In certain embodiments, the TNF superfamily protein is a protein selected from the group consisting of TRAIL, TL1A, DR3, DR4 (TRAILR1), DR5 (TRAILR2), DR1 (TRAILR3), DR2 (TRAILR4), FasL, Fas, TNFα, TNFβ, TNFγ, TNFR1, TNFR2, 4-1BBL, 4-1BB, OX40L, OX40, CD40L, CD40, GITRL, GITR, CD70, CD27, LIGHT, HVEM, CD30L (CD153), CD30, BAFF, APRIL, BAFFR, BCMA, TACI, RANKL, RANK, TWEAK and TWEAKR. In certain embodiments, the trimerization motif-comprising domain of the fusion polypeptide of FV-114 and FV-20a to 28d above comprises a trimerization motif-comprising polypeptide or fragment thereof TRAIL, TL1A, FasL, TNFα, F4-1BBL, OX40L, CD40L, GITRL, CD70, or LIGHT. In certain embodiments, the trimerization motif-comprising domain of the fusion polypeptide of FV-114 and FV-20a to 28d above comprises an amino acid sequence selected from the group consisting of SEQ ID NO: 242 to 253.

In some embodiments, the linker of the protein for example as configured in an exemplary format described in FV-20a to 28d and FV-114 above, comprises an amino acid sequence of (GGGGS)n, wherein n=1, 2, 3, 4, 5, or 6. In some embodiments, the linker of the protein, for example as configured in an exemplary format described in FV-3 to 6, 29a to 47b, 48 to 78, and 128 to 190 above, comprises an amino acid sequence of GGGGSGGGGS, GGGGSGGGGSGOGGS or GGGGSGGGGSGGGGSGGGGS.

### Protein Drug Conjugate

In one aspect, the present disclosure provides a protein drug conjugate comprising a SIRP IgV monomer of present disclosure, a SIRP IgV multimer polypeptide of present disclosure comprising two or more SIRP IgV monomers, a fusion polypeptide of present disclosure comprising a CD47-binding SIRP IgV domain and a non-CD47 binding domain, or a Fe fusion protein, an antibody fusion protein, a Fab fusion protein or other fusion protein of present disclosure comprising one or more of the fusion polypeptides of present disclosure. In some embodiments, the protein drug conjugate comprises at least one conjugated moiety selected from the group consisting of a cytotoxic agent, a cytostatic agent, a radioactive isotope or compound, a chelator, a calreticulin-inducing agent, an immune stimulatory adjuvant moiety, a steroid, an immunosuppressor, DNA, RNA, a photosensitizer, a toxin, and an enzyme/pro-drug converting enzyme. In some embodiments, the protein conjugate comprises a SIRP IgV monomer, wherein the SIRP IgV monomer comprises an amino acid sequence selected from the group consisting of SEQ ID NO: 3 to 81. In some embodiments, the protein conjugate comprises a SIRP IgV monomer, wherein the SIRP IgV monomer comprises a mutation of K53H, R69H, Q52H, K68H, or Q52H+K68H. In some embodiments, the protein conjugate comprises a SIRP IgV monomer, wherein the SIRP IgV monomer comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the SIRP IgV monomer further comprises one or more substitutions selected from the group consisting of E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P350, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, E54D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113D/V114D/L114D, V113E/V114E/L114E, R114H/R115H/G115H, R114E/R115E/G115E, R114D/R115D/G115D, K116H/K117H, K116E/K117E, and K116D/K117D, wherein the “/” indicates different alternative mutation change at the corresponding amino acid residue position across SIRPα, SIRPβ and SIRPγ IgV domains, due to different parent amino acid residues and/or position numbering for the mutation at the corresponding position, according to the SIRPα, SIRPβ and SIRPγ IgV amino acid sequences provided in SEQ ID NO: 1 to 21. In some embodiments, the protein conjugate comprises a SIRP IgV monomer, wherein the SIRP IgV monomer comprises a mutation of K53H and one additional substitution selected from the group consisting of Q52H, K68H, E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, E54D, E54Q, E54N, E54P, ES45, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, I76H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, 097H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D. In some embodiments, the protein conjugate comprises a SIRP IgV monomer, wherein the SIRP IgV monomer comprises a mutation of R69H and one additional substitution selected from the group consisting of Q52H, K68H, E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31 Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, NSIH, N51D, N51E, Q52E, Q52D, Q52G, K53R, K53E, K53D, ES4H, E54D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79B/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D. In some embodiments, the protein conjugate comprises a SIRP IgV monomer, wherein the SIRP IgV monomer comprises a mutation of Q52H and one additional substitution selected from the group consisting of K53H, K68H, R69H, E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33B, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, ES4H, E54D, E54Q, ES4N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L660, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, I76H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D. In some embodiments, the protein conjugate comprises a SIRP IgV monomer, wherein the SIRP IgV monomer comprises a mutation of K68H and one additional substitution selected from the group consisting of Q52H, K53H, R69H, E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, B47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, ES4H, E54D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, I76H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103I/F104I, F103V/F104V, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D.

In some embodiments, the protein conjugate comprises a SIRP IgV multimer polypeptide comprising two SIRP IgV monomers, wherein the SIRP IgV monomer comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H. In some embodiments, the protein conjugate comprises a SIRP IgV multimer polypeptide comprising two SIRP IgV monomers, wherein the SIRP IgV multimer polypeptide comprises an amino acid sequence selected from the group consisting of SEQ ID NO: 82 to 96 and SEQ ID NO: 418 to 425. In some embodiments, the protein conjugate comprises a SIRP IgV multimer polypeptide comprising three SIRP IgV monomers, wherein the SIRP IgV monomer comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H. In some embodiments, the protein conjugate comprises a SIRP IgV multimer polypeptide comprising three SIRP IgV monomers, wherein the SIRP IgV multimer polypeptide comprises an amino acid sequence selected from the group consisting of SEQ ID NO: 97 to 109 and SEQ ID NO: 430 to 435. In some embodiments, the protein conjugate comprises a SIRP IgV multimer polypeptide comprising four SIRP IgV monomers, wherein the SIRP IgV monomer comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H. In some embodiments, the protein conjugate comprises a SIRP IgV multimer polypeptide comprising four SIRP IgV monomers, wherein the SIRP IgV multimer polypeptide comprises an amino acid sequence selected from the group consisting of SEQ ID NO: 110 to 113.

In some embodiments, the protein conjugate comprises two SIRP IgV monomers, wherein the SIRP IgV monomer comprises a mutation of K53H, R69H, Q52H, K68H, or Q52H+K68H. In some embodiments, the protein conjugate comprising two SIRP IgV monomers is configured in a format selected from the group consisting of FV-1, 2, 8 and 9 as set forth in FIG. 2, FV-48, 49, 58 and 61 as set forth in FIG. 5, FV-68, 71, 72, 94 and 98 as set forth in FIG. 6, FV-102, 106 and 109 as set forth in FIG. 7, FV-129 and 133 as set forth in FIG. 9, FV-136, 137, 143 and 144 as set forth in FIG. 10, FV-157, 160 and 161 as set forth in FIG. 11, and FV-169, 173, 177, 180, 183 and 184 as set forth in FIG. 12, wherein the SIRP IgV monomer comprises a mutation of K53H. In some embodiments, the protein conjugate comprising two SIRP IgV monomers of present disclosure in total is configured in a format selected from the group consisting of FV-1, 2, 8 and 9 as set forth in FIG. 2, FV-48, 49, 58 and 61 as set forth in FIG. 5, FV-68, 71, 72, 94 and 98 as set forth in FIG. 6, FV-102, 106 and 109 as set forth in FIG. 7, FV-129 and 133 as set forth in FIG. 9, FV-136, 137, 143 and 144 as set forth in FIG. 10, FV-157, 160 and 161 as set forth in FIG. 11, FV-169, 173, 177, 180, 183 and 184 as set forth in FIG. 12, wherein the SIRP IgV monomer comprises a mutation of R69H. In some embodiments, the protein conjugate comprising two SIRP IgV monomers of present disclosure in total is configured in a format selected from the group consisting of FV-1, 2, 8 and 9 as set forth in FIG. 2, FV-48, 49, 58 and 61 as set forth in FIG. 5, FV-68, 71, 72, 94 and 98 as set forth in FIG. 6, FV-102, 106 and 109 as set forth in FIG. 7, FV-129 and 133 as set forth in FIG. 9, FV-136, 137, 143 and 144 as set forth in FIG. 10, FV-157, 160 and 161 as set forth in FIG. 11, FV-169, 173, 177, 180, 183 and 184 as set forth in FIG. 12, wherein the SIRP IgV monomer comprises a mutation of Q52H. In some embodiments, the protein conjugate comprising two SIRP IgV monomers of present disclosure in total is configured in a format selected from the group consisting of FV-1, 2, 8 and 9 as set forth in FIG. 2, FV-48, 49, 58 and 61 as set forth in FIG. 5, FV-68, 71, 72, 94 and 98 as set forth in FIG. 6, FV-102, 106 and 109 as set forth in FIG. 7, FV-129 and 133 as set forth in FIG. 9, FV-136, 137, 143 and 144 as set forth in FIG. 10, FV-157, 160 and 161 as set forth in FIG. 11, FV-169, 173, 177, 180, 183 and 184 as set forth in FIG. 12, wherein the SIRP IgV monomer comprises a mutation of K68H. In some embodiments, the protein conjugate comprising two SIRP IgV monomers of present disclosure in total is configured in a format selected from the group consisting of FV-1, 2, 8 and 9 as set forth in FIG. 2, FV-48, 49, 58 and 61 as set forth in FIG. 5, FV-68, 71, 72, 94 and 98 as set forth in FIG. 6, FV-102, 106 and 109 as set forth in FIG. 7, FV-129 and 133 as set forth in FIG. 9, FV-136, 137, 143 and 144 as set forth in FIG. 10, FV-157, 160 and 161 as set forth in FIG. 11, FV-169, 173, 177, 180, 183 and 184 as set forth in FIG. 12, wherein the SIRP IgV monomer comprises a mutation of Q52H+K68H.

In some embodiments, the protein conjugate comprises four SIRP IgV monomers, wherein the SIRP IgV monomer comprises a mutation of K53H, R69H, Q52H, K68H, or Q52H+K68H. In some embodiments, the protein conjugate comprising four SIRP IgV monomers is configured in a format selected from the group consisting of FV-3, 4, 5, 10 and 12 as set forth in FIG. 2, FV-50, 51, 54, 59 and 62 as set forth in FIG. 5, FV-70, 76, 77, 78, 96 and 100 as set forth in FIG. 6, FV-104, 108 and 111 as set forth in FIG. 7, FV-I31 and 135 as set forth in FIG. 9, FV-138, 139, 142, 145, 146 and 149 as set forth in FIG. 10, FV-159, 165, 166 and 167 as set forth in FIG. 11, and FV-171, 175, 179, 181, 189 and 190 as set forth in FIG. 12, wherein the SIRP IgV monomer comprises a mutation of K53H. In some embodiments, the protein conjugate comprising four SIRP IgV monomers is configured in a format selected from the group consisting of FV-3, 4, 5, 10 and 12 as set forth in FIG. 2, FV-50, 51, 54, 59 and 62 as set forth in FIG. 5, FV-70, 76, 77, 78, 96 and 100 as set forth in FIG. 6, FV-104, 108 and 111 as set forth in FIG. 7, FV-I31 and 135 as set forth in FIG. 9, FV-138, 139, 142, 145, 146 and 149 as set forth in FIG. 10, FV-159, 165, 166 and 167 as set forth in FIG. 11, and FV-171, 175, 179, 181, 189 and 190 as set forth in FIG. 12, wherein the SIRP IgV monomer comprises a mutation of R69H. In some embodiments, the protein conjugate comprising four SIRP IgV monomers is configured in a format selected from the group consisting of FV-3, 4, 5, 10 and 12 as set forth in FIG. 2, FV-50, 51, 54, 59 and 62 as set forth in FIG. 5, FV-70, 76, 77, 78, 96 and 100 as set forth in FIG. 6, FV-104, 108 and 111 as set forth in FIG. 7, FV-I31 and 135 as set forth in FIG. 9, FV-138, 139, 142, 145, 146 and 149 as set forth in FIG. 10, FV-159, 165, 166 and 167 as set forth in FIG. 11, and FV-171, 175, 179, 181, 189 and 190 as set forth in FIG. 12, wherein the SIRP IgV monomer comprises a mutation of Q52H. In some embodiments, the protein conjugate comprising four SIRP IgV monomers is configured in a format selected from the group consisting of FV-3, 4, 5, 10 and 12 as set forth in FIG. 2, FV-50, 51, 54, 59 and 62 as set forth in FIG. 5, FV-70, 76, 77, 78, 96 and 100 as set forth in FIG. 6, FV-104, 108 and 111 as set forth in FIG. 7, FV-I31 and 135 as set forth in FIG. 9, FV-138, 139, 142, 145, 146 and 149 as set forth in FIG. 10, FV-159, 165, 166 and 167 as set forth in FIG. 11, and FV-171, 175, 179, 181, 189 and 190 as set forth in FIG. 12, wherein the SIRP IgV monomer comprises a mutation of K68H. In some embodiments, the protein conjugate comprising four SIRP IgV monomers is configured in a format selected from the group consisting of FV-3, 4, 5, 10 and 12 as set forth in FIG. 2, FV-50, 51, 54, 59 and 62 as set forth in FIG. 5, FV-70, 76, 77, 78, 96 and 100 as set forth in FIG. 6, FV-104, 108 and 111 as set forth in FIG. 7, FV-I31 and 135 as set forth in FIG. 9, FV-138, 139, 142, 145, 146 and 149 as set forth in FIG. 10, FV-159, 165, 166 and 167 as set forth in FIG. 11, and FV-171, 175, 179, 181, 189 and 190 as set forth in FIG. 12, wherein the SIRP IgV monomer comprises a mutation of Q52H+K68H.

Techniques for conjugating various types of therapeutic agents (e.g. cytotoxic or cytostatic small molecules, TLR agonists, STING agonists, radioactive compounds or chelators, steroid, DNA, RNA, photosensitizer, toxins and enzymes) to proteins, especially to antibodies, are well known in the arts, for example, as described in patents such as WO2021067776A2, WO2019108733A2, U.S. Pat. No. 10,155,821B2 U.S. Pat. No. 9,808,537B2, WO2021257525A1, WO2017180842A1, WO2018009916A1, WO2020190725A1, WO2021202984A1, U.S. Pat. No. 7,259,249B2, WO2018189382A1, WO2020081744A1, WO2021174091A1, U.S. Pat. No. 10,487,149B2, U.S. Pat. No. 8,524,239B2, WO2011097513A1, US20170002074A1, WO2022170971A1, WO2022228497A1 WO2017210471A1 and WO2022171101A1, as well as in journal articles such as (Khongorzul et al., Mol Cancer Res, 2020, Tsuchikama et al., Protein Cell, 2018, Yao et al., Int J Mol Sci, 2016, Gauzy-Lazo et al., SLAS Discov, 2020, Thakral et al., Indian J Med Res, 2014, Morais et al., Drug Discoy Today Technol, 2018, Sumerdon et al., Int J Rad Appl Instrum B, 1990, Winston et al., Curr Protoc Mol Biol, 2001, Walsh et al., Chem Soc Rev, 2021, Vitetta et al., Science, 1987), all incorporated herein by reference in its entirety and for all purposes. The therapeutic agent can be conjugated to the protein or antibody by chemical conjugation or enzymatic conjugation in a site-specific or non-specific manner, using cleavable linkers (e.g. acid-labile linker, peptidase-sensitive linker, photolabile linker, dimethyl linker or disulfide-containing linker, tumor-enriching linker) or non-cleavable linkers (e.g. maleimido-alkylene or maleimide-aryl linker) known in the arts, for example, as described in patents such as U.S. Pat. Nos. 7,745,394, 5,208,020, US20070092940, EP1968635B1, WO2014140317, EP1370298B1, WO2014202775, U.S. Pat. No. 10,947,317B2, U.S. Pat. No. 8,685,383B2, US20120183566, U.S. Pat. No. 9,669,107B2, U.S. Pat. No. 10,745,488B2 and WO2022170971A1, as well as in journal articles such as (Walsh et al., Chem Soc Rev, 2021, Yao et al., Int J Mol Sci, 2016, Tsuchikama et al., Protein Cell, 2018, Yurkovetskiy et al., Mol Cancer Ther, 2021), all incorporated herein by reference in its entirety and for all purposes. Furthermore, anti-CD47 antibody-drug conjugates comprising non-pH sensitive anti-CD47 antibody have been successfully generated using different cytotoxic payloads and conjugation technologies known in the arts, as described above, and showed potent anti-tumor activity against CD47-expressing tumor cells in vitro and CD47-expressing tumors in xenograft mouse models in vivo in preclinical studies (see {Si, 2021 #890}, and {Chiang, 2022 #891})

In some embodiments, the therapeutic agent can be conjugated to the protein in a manner that reduces its activity unless it is cleaved off the antibody, for example, by hydrolysis, by reduction, by a cleaving agent or by proteolytic degradation. In some applications, the therapeutic agent is conjugated to the antibody with a cleavable linker that is sensitive to cleavage within intracellular environment but is not substantially sensitive to the extracellular environment. Thus, the conjugate is cleaved off the protein or antibody after it is internalized by the target cell, for example, in the endosomal, lysosomal and/or caveolear environment by virtue of pH sensitivity, protease sensitivity or reduction sensitivity. Protease sensitive linker can be a peptidyl linker (e.g. a linker comprising a Val-Cit or Phe-Leu peptide) that is cleaved by an intracellular peptidase or protease enzyme, including a lysosomal or endosomal protease, such as cathepsins B and D and plasmin that are highly expressed in tumor tissues, e.g. as described in the reference (Dubowchik et al., Pharmacol Ther, 1999). pH-sensitive linker, for example, an acid-labile linker such as a hydrazone, semicarbazone, thiosemicarbazone, cis-aconitic amide, orthoester, acetal, ketal, or the like, is hydrolyzable under acidic conditions at usually pH<5.5 such as in lyosomes but relatively stable under neutral pH conditions such as in blood, as described in patent such as U.S. Pat. Nos. 5,122,368, 5,824,805 and 5,622,929. Reduction-sensitive linker (e.g. a disulfide linker) is cleavable under reducing conditions Disulfide linkers can be formed using SATA (N-succinimidyl-S-acetylthioacetate), SPDP (N-succinimidyl-3-(2-pyridyldithio)propionate), SPDB (N-succinimidyl-3-(2-pyridyldithio) butyrate) and SMPT (N-succinimidyl-oxycarbonyl-alpha-methyl-alpha-(2-pyridyl-dithio) toluene), SPDB and SMPT, as described in patent U.S. Pat. No. 4,880,935A and the reference such as (Thorpe et al., Cancer Res, 1987). In some applications, the therapeutic agent is conjugated to the antibody with a cleavable linker that is sensitive to cleavage within specific extracellular environment of disease tissues such as tumor, but is not substantially sensitive to the peripheral circulation environment and normal tissue environment, such that the conjugate is cleaved from the antibody after it's delivered into the extracellular environment of target disease tissue. A number of protease enzymes such as matriptase (MT-SP1), matrix metalloproteases (e.g. MMP2, MMP9, MMP7 et al.), uPA, ADAMs (e.g. ADAM10, ADAM17) and cathepsins are enriched in the extracellular environment of tumor than peripheral circulation and normal tissues (see e.g. (Vizovisek et al., Int J Mol Sci, 2021), and linkers sensitive to cleavage by such tumor enriched protease enzymes are suitable for extracellular cleavage of the conjugated therapeutic agent in tumor microenvironment. In some application, the therapeutic agent can also be conjugated to the antibody with a non-cleavable linker, such as a maleimido-alkylene or maleimide-aryl linker that is directly attached to the therapeutic agent and released by proteolytic degradation of the antibody.

In some embodiments, the conjugated moiety comprises a cytotoxic agent selected from the group consisting of tubulin inhibitors, DNA topoisomerase inhibitors, DNA minor groove binders, DNA alkylating agents, DNA intercalating agents, RNA polymerase inhibitors, spliceosome inhibitors and nicotinamide phosphoribosyltransferase inhibitors (NAMPTi). In certain embodiments, the foregoing cytotoxic agents comprise cytotoxic agents including but not limiting to auristatins (such as MMAE, MMAF, Auristatin F, Amberstatin, Auristatin W, dolastatin and dolaflexin, see e.g. patents U.S. Pat. Nos. 6,884,869, 5,635,483, 5,780,588, 7,498,298 and 8,685,383B2), maytansinoids (such as DM1, DM2, DM3 and DM4, see e.g. patent U.S. Pat. Nos. 5,208,020, 5,416,064 and EP0425235B1), tubulysins (such as AZ13599185, see e.g. patent US2015141646), taxanes (such as docetaxel, paclitaxel, larotaxel, tesetaxel, and ortataxel, see e.g. patent U.S. Pat. No. 7,390,898), trichothecene, vinca alkaloids (such as vindesine), methotrexate; camptothecins (such as SN-38, exatecan, DX-8951, Dxd, irrinotecan, topotecan, belotecan and camptothecin, see e.g. patents U.S. Pat. No. 10,155,821B2, U.S. Pat. No. 9,808,537B2), etoposides (such as etoposide and teniposide), calicheamicins (such as CM1, see e.g. patents U.S. Pat. Nos. 5,714,586 and 5,712,374), anthracycline (such as doxorubicin, daunorubicin, epirubicin, idarubicin and PNU-159682, see e.g. patents U.S. Pat. No. 6,630,579B2 and U.S. Pat. No. 8,900,589), duocarmycins (such as seco-DUBA and CC-1065, see e.g. patents EP2560645A2 and U.S. Pat. No. 5,475,092), benzodiazepines (such as pyrrolo[1,4]benzodiazepines, indolinobenzodiazepines, and oxazolidinobenzodiazepines, including pyrrolo[1,4]benzodiazepine dimers, indolinobenzodiazepine dimers, and oxazolidinobenzodiazepine dimmers, see e.g. patents U.S. Pat. No. 8,765,740, EP2766048B1, US2013028919 and US20110256157), amatoxins (such asα-manitin, see e.g. patent EP2436398B1), thailanstatin A and spliceostatins.

In one embodiment, the conjugated moiety comprises a MMAE. In additional embodiment, the MMAE payload is conjugated to the protein with a valine-citrulline (VC) dipeptide linker, as described in patents U.S. Pat. Nos. 7,745,394 and 6,884,869. In another embodiment, the conjugated moiety comprises Dxd. In additional embodiments, the Dxd payload is conjugated to the protein with a glycyl-glycyl-phenylalanyl-glycine (GGFG) tetrapeptide linker, as described in patents U.S. Pat. No. 10,155,821B2 and U.S. Pat. No. 9,808,537B2. In another embodiment, the conjugated moiety comprises PNU-159682. In additional embodiments, the PNU-159682 payload is conjugated to the protein with a succinimidyl 4-(N-maleimidomethyl)cyclohexane-1-carboxylate (SMCC) linker, as described in patent U.S. Pat. No. 8,389,697B2.

In certain embodiments, the antigen binding domain of the antibody drug conjugate Trastuzumab deruxtecan (DS-8201) comprising a Dxd payload (see U.S. Pat. No. 10,155,821B2 and U.S. Pat. No. 9,808,537B2), DB-1303 (DualityBio) comprising a toposiomeriase 1 inhibitor payload, SKB264 comprising a toposiomeriase I inhibitor payload, enfortumab vedotin comprising a MMAE payload, ARX788 comprising a AS269 payload, and trastuzumab duocarmazine (SYD985) comprising a DUBA/duocarmycin payload, can be replaced by a pH-sensitive SIRP IgV domain of the present disclosure to generate a protein drug conjugate targeting CD47.

In some embodiments, the conjugated moiety comprises a radioactive isotope or compound selected from the group consisting of ²²⁵Ac, ²¹¹At, ²¹²Bi, ⁶²Cu, ⁶⁴Cu, ⁶⁷Cu, ¹⁸F, ⁶⁶Ga, ⁶⁷Ga, ⁶⁸Ga, ¹²³I, ¹²⁵I, ¹³¹I, ¹¹¹In, ¹⁷⁷Lu, ¹⁵O, ²¹²Pb, ¹⁸⁶Re, ¹⁸⁸Re, ⁴⁴⁵c, ¹⁴⁹Tb, ¹⁵²Tb, ¹⁵⁵Tb, ¹⁶¹Tb, ⁹⁰Y and ⁸⁹Zr.

In some embodiments, the conjugated moiety comprises a chelator selected from the group consisting of 1,4,7,10-Tetraazacyclododecane-1,4,7,10-tetraacetic acid (DOTA), DOTATATE, DOTA-Bn, DO2A, Diethylenetriamene pentaacetate (DTPA), 6-amino-1,4-diazepine-triacetate (DATA), PCTA, 1,4,7-triazacyclononane-N,N′,N″-triacetic acid (NOTA), NOTP, TRAP, 1,4,7-Triazacyclononane (TACN), 6-amino-6-methylperhydro-1,4-diazepinetetraacetic acid (AAZTA), H4pypa, H2dedpa, H2CHXhox, H2hox, HBED, and DFO, as described in the reference of chelators (Sneddon et al., Curr Opin Chem Biol, 2021). In certain embodiments, the chelator preferentially comprises DOTA, DOTATATE, or DOTA-Bn. In additional embodiments, the foregoing chelator optionally chelates with ¹⁷⁷Lu. As an example, anti-CD47 antibody conjugated with ⁶⁸Ga, ⁸⁹Zr or ¹⁷⁷Lu through NOTA, DFO or DOTA have all been successfully generated using the technologies known in the arts and reported as effective theranostics for tumor imaging and therapy (see {Zhang, 2023 #892}).

In some embodiments, the conjugated moiety comprises a calreticulin-inducing agent selected from the group consisting of anthracyclin such as doxorubicin, doxorubicin, daunorubicin, epirubicin, idarubicin and mitoxantrone, and a PPI/GADD34 inhibitor such as tautomycin, calyculin A and salubrinal, or fullerenols, that induce translocation of calrecticulin to the cell surface, as described in the reference such as (Obeid et al., Immunol Rev, 2007, Obeid et al., Nat Med, 2007, Kui Chen, Nano Today, 2021). In additional embodiments, the calreticulin-inducing agent synergizes with CD47-blockade to enhance phagocytosis of the CD47-expressing target cells, as described in the reference such as (Chao et al., Sci Transl Med, 2010, Feng et al., Nat Commun, 2018, Obeid et al., Nat Med, 2007).

In some embodiments, the conjugated moiety comprises an agonist to a pattern recognition receptor (PRR) for pathogen-associated molecular patterns (PAMPs) or damage-associated molecular patterns (DAMPs). In certain embodiments, the foregoing pattern recognition receptors include but are not limited to, Toll-like receptors (TLRs), STimulator of INterferon Genes (STING), C-type lectin receptors (CLRs), Rig-I-like receptors (RLRs) and NOD-like receptors (NLRs), as described in reference such as (Li et al., Signal Transduct Target Ther, 2021).

In certain embodiments, the conjugated moiety comprises TLR2 agonists including but not limiting to lipopeptide, peptide and small molecule agonists CBLB612, SV-283, ISA-201, Pam3Cys, Pam3Cys-Ser-(Lys) 4 (Pam3CSK4), Triacyl lipid A (OM-174), Lipoteichoic acid (LTA), peptidoglycan, and CL419 (S-(2,3-bis(palmitoyloxy)-(2RS)propyl)-(R)-cysteinyl spermine), TLR2/6 agonists including but not limiting to Pam2CSK4, and TLR2/7 agonists including but not limiting to CL572, CL413, and CL401, that are known in the arts, for example, as described in patents U.S. Pat. No. 10,675,358B2 and US20170158772A1 and the reference (Anwar et al., Med Res Rev, 2019).

In certain embodiments, the conjugated moiety comprises TLR4 agonists including but not limiting to lipopolysaccharide (LPS), monophosphoryl lipid A (MPLA) and small molecule agonists GSK1795091, glycopyranosyl lipid (GLA)-SE/GLA-AF/G-305 (Immune Design), G100, PEPA-10, PET-lipid A (Cascadian Therapeutics), and MPL (Allergy Therapeutics), that are known in the arts, for example, as described in patents U.S. Pat. No. 10,675,358B2 and US20170158772A1 and the reference (Anwar et al., Med Res Rev, 2019).

In certain embodiments, the conjugated moiety comprises TLR5 agonists including but not limiting to flagellin and recombinant protein agonist thereof including mobilan, entolimod, VAX125 and VAX102, that are known in the arts, for example, as described in patents U.S. Pat. No. 10,675,358B2 and US20170158772A1 and the reference (Anwar et al., Med Res Rev, 2019).

In certain embodiments, the conjugated moiety comprises TLR3 agonists including but not limiting to dsRNA or polyinosine-polycytidylic acid (poly(I:C)), poly-ICLC, Polyadenylic-polyuridylic acid (poly(A: U), and poly(I)-poly(C12U), that are known in the arts, for example, as described in patents U.S. Pat. No. 10,675,358B2 and US20170158772A1 and the reference (Anwar et al., Med Res Rev, 2019).

In certain embodiments, the conjugated moiety comprises TLR7 agonists including but not limiting to imidazoquinoline type and other small molecule agonists including imiquimod, Gardiquimod, loxoribine, GSK2245035, 852A, GS-9620, RO6864018, RO7020531, CL264, CL307, 852A, BNT411, DSP-0509, LHC165, NJH395, RO7119929 and TQ-A3334, that are known in the arts, for example, as described in patents U.S. Pat. No. 10,675,358B2 and US20170158772A1 and the reference (Anwar et al., Med Res Rev, 2019, Frega et al., Oncoimmunology, 2020).

In certain embodiments, the conjugated moiety comprises TLR8 agonists including but not limiting to small molecule agonists motolimod, IRM1, IRM2, IRM3, TL8-506 and the TLR8 agonist of SBT6050, that are known in the arts, for example, as described in patents U.S. Pat. No. 10,675,358B2 and US20170158772A1 and the reference (Anwar et al., Med Res Rev, 2019, Frega et al., Oncoimmunology, 2020).

In certain embodiments, the conjugated moiety comprises TLR7/8 agonists including but not limiting to small molecule agonists resiquimod, MEDI9197, T785, BDB001, BDB018, BDB030, CV8102, NKTR-262, CL097, CL075 and the TLR7/8 agonist moiety of BDC-1001, that are known in the arts, for example, as described in patents U.S. Pat. No. 10,675,358B2 and US20170158772A1 and the reference (Anwar et al., Med Res Rev, 2019, Frega et al., Oncoimmunology, 2020). In particular embodiments, the TLR7/8 agonist comprises BDB001, BDB018, or the TLR7/8 agonist moiety of BDC-1001.

In certain embodiments, the conjugated moiety comprises TLR9 agonists including but not limiting to single strand CpG oligodeoxynucleotides (CpG ODN) and oligonucleotide-based agonists MGN1703, SD-101, CYT003, DUK-CpG-001, CpG-7909, GNKG168, EMD1202081, IMO-2125, CpG10104, and AZD1419, as well as the TLR9 agonist moiety of the antibody-drug conjugate TAC-001 and ALTA-002, that are known in the arts, as patents U.S. Pat. No. 10,675,358B2, US20170158772A1, US20220056069A1, for example, described in WO2018189382A1 and WO2020081744A1 and the reference (Anwar et al., Med Res Rev, 2019).

In certain embodiments, the conjugated moiety comprises cytosolic DNA and cyclic dinucleotides (CDN) STING agonists including but not limiting to ADU-S100, Cyclic[G(2′,5)p] (2′,5′)p] (2′2′-cGAMP), cyclic [G(2′,5′)pA(3′,5′)p] (2′3′-cGAMP), cyclic[G(3′,5′)pA(3′,5′)p] (3′3′-cGAMP), Cyclic di-adenylate monophosphate c-di-AMP), 2′,5′-3′,5′-c-diAMP (2′3′-c-di-AMP), Cyclic di-guanylate monophosphate (c-di-GMP), 2′,5′-3′,5′-c-diGMP (2′3′-c-di-GMP), Cyclic di-inosine monophosphate (c-di-IMP), Cyclic di-uridine monophosphate (c-di-UMP), 3′3′-cyclic-AIMP, BMS-986301, GSK532, JNJ-4412, MK-1454, SB11285. In certain embodiments, the conjugated moiety comprises non-CDN small molecule STING agonists including but not limiting to ALG-031048, E7766, JNJ-6196, MK-2118, MSA-1, MSA-2, SNX281, SR-717, TAK676 and TTI-10001, and the STING agonist moiety of the antibody-drug conjugate XMT-2056 and CRD-5500, that are known in the arts, for example, as described in patents U.S. Pat. No. 10,675,358B2, US20170158772A1, WO2021026009A1 and WO2021202984A1 and the reference (Amouzegar et al., Cancers (Basel), 2021, Yan et al., Vaccines (Basel), 2021).

In certain embodiments, the antigen binding domain of the antibody drug conjugate BDC-1001 comprising a TLR7/8 agonist moiety (see WO2018009916A1 and WO2020190725A1), SBT6050 comprising a TLR8 agonist moiety, TAC-001 and ALTA-002 each comprising a TLR9 agonist moiety (see WO2018189382A1, WO2020081744A1 and WO2021174091A1), as well as XMT-2056 and CRD-5500 each comprising a STING agonist moiety (see WO2021202984A1), can be replaced by a SIRP IgV domain of present disclosure to generate a protein drug conjugate targeting CD47.

In certain embodiments, the conjugated moiety comprises NLR agonists including but not limiting to acylated derivative of iE-DAP, D-gamma-Glu-mDAP, L-Ala-gamma-D-Glu-mDAP, Muramyldipeptide with a C18 fatty acid chain, Muramyldipeptide, muramyl tripeptide, and N-glycolylated muramyldipeptide, that are known in the arts, for example, as described in patent U.S. Pat. No. 10,675,358B2.

In certain embodiments, the conjugated moiety comprises RIG-1 agonists including but not limiting to 5′ppp-dsRNA (5′-pppGCAUGCOACCUCUGUUUGA-3′: 3′-CGUACGCUGGAGACAAACU-5′), 3p-hpRNA, Poly(deoxyadenylic-deoxythymidylic) acid (Poly(dA: dT)), Poly(I: C), MK-4621 (RGT100), SLR14, SLR20, KIN700, KINI148, KIN600, KIN500, KIN100, KIN101, KIN400, KIN2000, and SB-9200, that are known in the arts, for example, as described in patents U.S. Pat. No. 10,675,358B2, US20170158772A1 and U.S. Pat. No. 8,871,799B2 and the reference (lurescia et al., Cancers (Basel), 2020).

In certain embodiments, the conjugated moiety comprises photosensitive agents including but not limiting to silicon phthalocyanine dye such as IRDye700DX, that are known in the arts, for example, as described in patent U.S. Pat. No. 8,524,239B2 and the reference (Maczynska et al., Cell Death Dis, 2020).

In certain embodiments, the conjugated moiety comprises a protein toxin, or an enzymatically active toxin of bacterial, fungal, plant, or animal origin, or fragments thereof. In certain embodiments, the toxins include but are not limited to, diphtheria A chain, nonbinding active fragments of diphtheria toxin, exotoxin A chain (from Pseudomonas aeruginosa), Shiga-like toxin A subunit, ricin A chain, abrin A chain, modeccin A chain, alpha-saicm, Aleurites fordii proteins, dianthin proteins, Phytolaca americana proteins (PAPI, PAPII, and PAP-S), Momordica charantiain ibitor, curcin, crotin, Sapaonaria officinalis inhibitor, gelonin, mitogellin, restrictocin, phenomycin, enomycin, the tricothecenes, Clostridium perfringens phospholipase C (PLC), bovine pancreatic ribonuclease (BPR), antiviral protein (PAP), abrin, cobra venom factor (CVF), gelonin (GEL), saporin (SAP), and viscumin, that are known in the arts, for example, as described in patent WO2011097513A1 and WO2019204272A1 and the reference (Kim et al., J Pharm Sci, 2020).

In certain embodiments, the conjugated moiety comprises an enzymes/pro-drug converting enzyme including but not limiting to alkaline phosphatases, arylsulfatases, cytosine deaminase, proteases such as serratia thermolysis, subtilisin, carboxy peptidases and protease, cathepsins, D-alanylcarboxypeptidases, glycosyltransferase, carbohydrate-cleaving enzymes such as B-galactosidase, neuraminidase and sialidase, β-lactamase, β-glucosidase, β-glucuronidase, penicillin amidases such as penicillin V amidase and penicillin G amidase, nitroreductase and carboxypeptidase A, that are known in the arts, for example, as described in patent US20170002074A1 and the reference (Sharma et al., Expert Opin Biol Ther, 2017) and (Neuberger et al., Nature, 1984). In certain embodiments, the conjugated moiety comprises a sialidase. In certain embodiments, the conjugated moiety comprises a glycosyltransferase.

In some embodiments, the conjugated moiety is covalently conjugated to cystein, lysine, carbohydrate glyco-group or other chemically active group of the protein or Fe through techniques known in the arts, for example, as described in patents such as U.S. Pat. Nos. 7,745,394, 5,208,020, US20070092940, EP1968635B1, WO2014140317, EP1370298B1, WO2014202775, U.S. Pat. No. 10,947,317B2, U.S. Pat. No. 8,685,383B2, US20120183566, U.S. Pat. No. 9,669,107B2, U.S. Pat. No. 10,745,488B2, WO2021067776A2, WO2019108733A2, U.S. Pat. No. 10,155,821B2, U.S. Pat. No. 9,808,537B2, WO2021257525A1, WO2017180842A1, WO2020190725A1, WO2018009916A1, WO2021202984A1, U.S. Pat. No. 7,259,249B2, WO2018189382A1, WO2020081744A1, WO2021174091A1, U.S. Pat. No. 10,487,149B2, U.S. Pat. No. 8,524,239B2, WO2011097513A1 and US20170002074A1, as well as in journal articles such as (Walsh et al., Chem Soc Rev, 2021, Yao et al., Int J Mol Sci, 2016, Tsuchikama et al., Protein Cell, 2018, Yurkovetskiy et al., Mol Cancer Ther, 2021, Morais et al., Drug Discov Today Technol, 2018, Thakral et al., Indian J Med Res, 2014, Vitetta et al., Science, 1987, Winston et al., Curr Protoc Mol Biol, 2001), all incorporated herein by reference in its entirety and for all purposes.

In some embodiments, the protein drug conjugate comprises in total one SIRP IgV monomer comprising a pH-sensitive mutation of K53H, R69H, Q52H, K68H or Q52H+K68H, or no said pH-sensitive mutation. In certain embodiments, the protein drug conjugate comprises in total one SIRP IgV monomer comprising an amino acid sequence selected from the group consisting of SEQ ID NO: 3 to 81. In some embodiments, the protein drug conjugate comprises in total two SIRP IgV monomers comprising a pH-sensitive mutation of K53H, R69H, Q52H, K68H or Q52H+K68H, or no said pH-sensitive mutation. In certain embodiments, the protein drug conjugate comprises in total two SIRP IgV monomers, wherein the two SIRP IgV monomers located in two different polypeptides comprising the same or different amino acid sequence selected from the group consisting of SEQ ID NO: 3 to 81. In certain embodiments, the protein drug conjugate comprises in total two SIRP IgV monomers comprising one SIRP IgV multimer polypeptide comprising two SIRP IgV monomers. In certain embodiments, the SIRP IgV multimer polypeptide comprising two SIRP IgV monomers comprises an amino acid sequence selected from the exemplary group consisting of SEQ ID NO: 82 to 96 and SEQ ID NO: 418 to 425. In some embodiments, the protein drug conjugate comprises in total three or four SIRP IgV monomers comprising a mutation of K53H or R69H. In certain embodiments, the protein drug conjugate comprises in total three or four SIRP IgV monomers comprising one or more separate SIRP IgV monomers located in different polypeptides, and/or one or more SIRP IgV multimer polypeptides comprising two, three or four SIRP IgV monomers. In certain embodiments, the protein drug conjugate comprises in total three or four SIRP IgV monomers comprising a SIRP IgV multimer polypeptide comprising three or four SIRP IgV monomers. In certain embodiments, the SIRP IgV multimer polypeptide comprising three or four SIRP IgV monomers comprises an amino acid sequence selected from the exemplary group consisting of SEQ ID NO: 97 to 113 and SEQ ID NO: 430 to 435.

In some embodiments, the protein drug conjugate is configured in a format selected from the group consisting of FV-1 to 6, FV-48 to 57, and FV-64 to 78. In some embodiments, the protein drug conjugate comprises in total one SIRP IgV monomer comprising a pH-sensitive mutation of K53H, R69H, Q52H, K68H or Q52H+K68H, or no said pH-sensitive mutation. In some embodiments, the protein drug conjugate comprises in total one SIRP IgV monomer comprising an amino acid sequence selected from the group consisting of SEQ ID NO: 3 to 81. In some embodiments, the protein drug conjugate comprises in total two SIRP IgV monomers comprising a pH-sensitive mutation of a mutation of K53H, R69H, Q52H, K68H or Q52H+K68H, or no said pH-sensitive mutation. In some embodiments, the protein drug conjugate comprises in total two SIRP IgV monomers, wherein the two SIRP IgV monomers on two different polypeptides comprising the same or different amino acid sequence selected from the group consisting of SEQ ID NO: 3 to 81. In some embodiments, the protein drug conjugate comprises in total two SIRP IgV monomers comprising one SIRP IgV multimer polypeptide comprising two SIRP IgV monomers. In certain embodiments, the SIRP IgV multimer polypeptide comprising two SIRP IgV monomers comprises an amino acid sequence selected from the group consisting of SEQ ID NO: 82 to 96 and SEQ ID NO: 418 to 425. In some embodiments, the protein drug conjugate comprises in total three or four SIRP IgV monomers comprising a mutation of K53H, R69H, Q52H, K68H or Q52H+K68H. In some embodiments, the protein drug conjugate comprises in total three or four SIRP IgV monomers comprising preferentially a mutation of K53H or R69H. In some embodiments, the protein drug conjugate comprises in total three or four SIRP IgV monomers comprising one or more separate SIRP IgV monomers on different polypeptides, and/or one or more SIRP IgV multimer polypeptides comprising two, three or four SIRP IgV monomers. In some embodiments, the protein drug conjugate comprises in total three or four SIRP IgV monomers comprising a SIRP IgV multimer polypeptide comprising three or four SIRP IgV monomers. In certain embodiments, the SIRP IgV multimer polypeptide comprising three or four SIRP IgV monomers comprises an amino acid sequence selected from the group consisting of SEQ ID NO: 97 to 113 and SEQ ID NO: 430 to 435.

### Nucleic Acid, Vector, Process, Pharmaceutical Compositions and Methods of Use

In some embodiments, a nucleic acid or nucleic acids comprise a sequence encoding a SIRP IgV monomer of present disclosure, a SIRP IgV multimer polypeptide of present disclosure comprising two or more SIRP IgV monomers, a fusion polypeptide of present disclosure comprising a CD47-binding SIRP IgV domain and a non-CD47 binding domain, or a Fc fusion protein, an antibody fusion protein, a Fab fusion protein or other fusion protein of present disclosure comprising one or more of the fusion polypeptides of present disclosure. In additional embodiments, the nucleic acid is a DNA or RNA. In another embodiments, a vector or vectors (e.g. cloning vector, and expression vector) comprise the foregoing nucleic acid or nucleic acids. In additional embodiments, the vector comprises a plasmid and/or a viral vector. In some embodiments, a host cell comprises one or more of the foregoing vectors. In some embodiments, a process for production of a polypeptide or protein of present disclosure, comprising culturing the host cell with the foregoing vector or vectors and isolating the polypeptide or protein. In some embodiments, a pharmaceutical composition comprises a SIRP IgV monomer of present disclosure, a SIRP IgV multimer polypeptide of present disclosure, a fusion polypeptide of present disclosure, a fusion protein of present disclosure, and/or a protein drug conjugate of present disclosure, and a pharmaceutically acceptable carrier.

In some embodiments, a method of treating a CD47-expressing disease in a mammal comprises administering an effective amount of a SIRP IgV monomer of present disclosure, a SIRP IgV multimer polypeptide of present disclosure, a fusion polypeptide of present disclosure, a fusion protein of present disclosure, and/or a protein drug conjugate of present disclosure, to a mammal in need thereof.

In some embodiments, a method of treating a CD47-expressing disease in a mammal comprising administrating an effective amount of a protein or protein conjugate comprising a CD47-binding SIRP IgV domain, wherein the protein or protein conjugate avoids or reduces platelet binding and/or thrombocytopenia, wherein the SIRP IgV domain of the protein or protein conjugate comprises a substitution of K53H, R69H, Q52H and/or K68H. In some embodiments, a method of treating a CD47-expressing disease in a mammal comprising administrating an effective amount of a protein or protein conjugate comprising a CD47-binding SIRP IgV domain, wherein the protein or protein conjugate avoids or reduces platelet binding and/or thrombocytopenia, wherein the SIRP IgV domain of the protein or protein conjugate comprises a substitution of K53H. In some embodiments, a method of treating a CD47-expressing disease in a mammal comprising administrating an effective amount of a protein or protein conjugate comprising a CD47-binding SIRP IgV domain, wherein the protein or protein conjugate avoids or reduces platelet binding and/or thrombocytopenia, wherein the SIRP IgV domain of the protein or protein conjugate comprises a substitution of R69H. In some embodiments, a method of treating a CD47-expressing disease in a mammal comprising administrating an effective amount of a protein or protein conjugate comprising a CD47-binding SIRP IgV domain, wherein the protein or protein conjugate avoids or reduces platelet binding and/or thrombocytopenia, wherein the SIRP IgV domain of the protein or protein conjugate comprises a substitution of Q52H. In some embodiments, a method of treating a CD47-expressing disease in a mammal comprising administrating an effective amount of a protein or protein conjugate comprising a CD47-binding SIRP IgV domain, wherein the protein or protein conjugate avoids or reduces platelet binding and/or thrombocytopenia, wherein the SIRP IgV domain of the protein comprises a substitution of K68H.

In some embodiments, a method of treating a CD47-expressing disease in a mammal comprising administrating an effective amount of a variant of TTI-621, wherein the variant of TTI-621 avoids or reduces platelet binding and/or thrombocytopenia, wherein TTI-621 comprises an amino acid sequence of SEQ ID NO: 300 and the variant of TTI-621 comprises a substitution of K53H, R69H, Q52H and/or K68H in its SIRP IgV domain. In some embodiments, a method of treating a CD47-expressing disease in a mammal comprising administrating an effective amount of a variant of TTI-621, wherein the variant of TTI-621 avoids or reduces platelet binding and/or thrombocytopenia, wherein TTI-621 comprises an amino acid sequence of SEQ ID NO: 300 and the variant of TTI-621 comprises a substitution of K53H in its SIRP IgV domain. In some embodiments, a method of treating a CD47-expressing disease in a mammal comprising administrating an effective amount of a variant of TTI-621, wherein the variant of TTI-621 avoids or reduces platelet binding and/or thrombocytopenia, wherein TTI-621 comprises an amino acid sequence of SEQ ID NO: 300 and the variant of TTI-621 comprises a substitution of R69H in its SIRP IgV domain. In some embodiments, a method of treating a CD47-expressing disease in a mammal comprising administrating an effective amount of a variant of TTI-621, wherein the variant of TTI-621 avoids or reduces platelet binding and/or thrombocytopenia, wherein TTI-621 comprises an amino acid sequence of SEQ ID NO: 300 and the variant of TTI-621 comprises a substitution of Q52H in its SIRP IgV domain. In some embodiments, a method of treating a CD47-expressing disease in a mammal comprising administrating an effective amount of a variant of TTI-621, wherein the variant of TTI-621 avoids or reduces platelet binding and/or thrombocytopenia, wherein TTI-621 comprises an amino acid sequence of SEQ ID NO: 300 and the variant of TTI-621 comprises a substitution of K68H in its SIRP IgV domain.

In some embodiments, a method of treating a CD47-expressing disease in a mammal comprising administrating an effective amount of a T cell engager comprising a SIRP IgV domain binding to CD47 and a CD3 binding domain that binds to and activate T cells, wherein the CD47-binding T cell engager avoids or reduces fratricide of CD47-positive T cells at physiological pH, wherein the SIRP IgV domain of the T cell engager comprises a mutation of K53H, R69H, Q52H and/or K68H. In some embodiments, a method of treating a CD47-expressing disease in a mammal comprising administrating an effective amount of a T cell engager comprising a SIRP IgV domain binding to CD47 and a CD3 binding domain that binds to and activate T cells, wherein the CD47-binding T cell engager avoids or reduces fratricide of CD47-positive T cells at physiological pH, wherein the SIRP IgV domain of the T cell engager comprises a mutation of R69H. In some embodiments, a method of treating a CD47-expressing disease using a T cell engager comprising a SIRP IgV domain binding to CD47 and a CD3 binding domain that binds to and activate T cells, wherein the CD47-binding T cell engager avoids or reduces fratricide of CD47-positive T cells at physiological pH, wherein the SIRP IgV domain of the T cell engager comprises a mutation of K53H. In some embodiments, a method of treating a CD47-expressing disease using a T cell engager comprising a SIRP IgV domain binding to CD47 and a CD3 binding domain that binds to and activate T cells, wherein the CD47-binding T cell engager avoids or reduces fratricide of CD47-positive T cells at physiological pH, wherein the SIRP IgV domain of the T cell engager comprises a mutation of Q52H. In some embodiments, a method of treating a CD47-expressing disease using a T cell engager comprising a SIRP IgV domain binding to CD47 and a CD3 binding domain that binds to and activate T cells, wherein the CD47-binding T cell engager avoids or reduces fratricide of CD47-positive T cells at physiological pH, wherein the SIRP IgV domain of the T cell engager comprises a mutation of K68H.

In some embodiments, the disease is a disease of cancer, fibrosis, atherosclerosis, inflammation or senescence. In certain embodiments, the disease is a disease of cancer, wherein the cancer is ovarian cancer, endometrial cancer, uterine cancer, cervical cancer, breast cancer, gastric cancer, esophageal cancer, lung cancer, head and neck cancer, colorectal cancer, liver cancer, bone cancer, sarcoma, osteosarcoma, brain cancer, multiple myeloma, acute myeloid leukemia (AML), myelodysplastic syndrome, adrenal cancer, anal cancer, bile duct cancer, bladder cancer, basal cell skin cancer, chondrosarcoma, Ewing's sarcoma, eye cancer, gallbladder cancer, gastrointestinal carcinoid tumors, gastrointestinal stromal tumor (GIST), glioma, glioblastoma, hepatocellular cancer, kaposi sarcoma, kidney cancer, laryngeal and hypopharyngeal cancer, leiomyosarcoma, non-small cell lung cancer (NSCLC), small cell lung cancer (SCLC), lung carcinoid tumor, mesothelioma, Merkel cell carcinoma, melanoma, nasal cavity and paranasal sinus cancer, nasopharyngeal cancer, neuroendocrine cancer, neuroblastoma, oral cavity and oropharyngeal cancer, pancreatic cancer, penile cancer, pituitary tumors, prostate cancer, renal cancer, retinoblastoma, rhabdomyosarcoma, salivary gland cancer, squamous cell skin cancer, small intestine cancer, stomach cancer, testicular cancer, thymus cancer, thyroid cancer, uterine sarcoma, urinary tract cancer, vaginal cancer, vulvar cancer, Wilms' tumor, diffuse large β-cell lymphoma (DLBCL), follicular lymphoma, bairy-cell leukemia, Hodgkin's lymphoma, leukemia, acute lymphocytic leukemia (ALL), chronic lymphocytic leukemia (CLL), chronic myeloid leukemia (CML), chronic myelomonocytic leukemia (CMML), lymphoma, Mantle cell lymphoma (MCL), marginal zone lymphoma (MZL), myeloma, Non-Hodgkin lymphoma (NHL), plasmacytoma, or Waldenstrom macroglobulinemia. In certain embodiments, the disease is a fibrotic disease, wherein the fibrotic disease comprises comprising fibrosis of lung, liver, heart, kidney, skin, eye, muscle and/or connective tissues, such as idiopathic pulmonary fibrosis, liver fibrosis in nonalcoholic fatty liver disease (NAFLD) and non-alcoholic steatohepatitis (NASH), scleroderma and Systemic Sclerosis. In certain embodiments of the method, the mammal is a human.

In some embodiments, a kit for diagnosis or treatment, said kit comprises a SIRP IgV monomer of present disclosure, a SIRP IgV multimer polypeptide of present disclosure, a fusion polypeptide of present disclosure, a fusion protein of present disclosure, and/or a protein drug conjugate of present disclosure, or the pharmaceutical composition thereof, and instruction for using it for diagnosis or treatment.

### Synthetic Receptor for Cell Therapy

In one aspect, the present disclosure provides a synthetic receptor for engineered cell therapy comprising a SIRP IgV monomer of present disclosure, a SIRP IgV multimer polypeptide of present disclosure comprising two or more SIRP IgV monomers, or a fusion polypeptide of present disclosure comprising a CD47-binding SIRP IgV domain and a non-CD47 binding domain. In some embodiments, the synthetic receptor comprises a chimeric antigen receptor (CAR), a synthetic T cell receptor (TCR), or a T cell-antigen coupler (TAC), wherein the synthetic receptor comprises an antigen binding domain comprising a SIRP IgV monomer, a SIRP IgV multimer polypeptide, or a SIRP IgV domain-comprising fusion polypeptide of present disclosure, a transmembrane domain and an intracellular signaling domain.

In some embodiments, the synthetic receptor comprises a SIRP IgV monomer, wherein the SIRP IgV monomer comprises an amino acid sequence selected from the group consisting of SEQ ID NO: 3 to 81. In some embodiments, the synthetic receptor comprises a SIRP IgV monomer, wherein the SIRP IgV monomer comprises a mutation of K53H, R69H, Q52H, K68H, or Q52H+K68H. In some embodiments, the synthetic receptor comprises a SIRP IgV monomer, wherein the SIRP IgV monomer comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H, wherein the SIRP IgV monomer further comprises one or more substitutions selected from the group consisting of E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, 13 IN/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, ES4D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D, wherein the “/” indicates different alternative mutation change at the corresponding amino acid residue position across SIRPα, SIRPβ and SIRPγ IgV domains, due to different parent amino acid residues and/or position numbering for the mutation at the corresponding position, according to the SIRPα, SIRP and SIRPγ IgV amino acid sequences provided in SEQ ID NO: 1 to 21. In some embodiments, the synthetic receptor comprises a SIRP IgV monomer, wherein the SIRP IgV monomer comprises a mutation of K53H and one additional substitution selected from the group consisting of Q52H, K68H, E3H/G3H, E3D/G3D, L4H, L4E, L4D, L4V, L4I, V6H/M6H, V6I/M6I, V6L/M6L, V6E, V6D, D10H/B10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, E54D, E54Q, E54N, ES4P, ES45, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, I76H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D. In some embodiments, the synthetic receptor comprises a SIRP IgV monomer, wherein the SIRP IgV monomer comprises a mutation of R69H and one additional substitution selected from the group consisting of Q52H, K68H, E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31 Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, ES4H, ES4D, ES4Q, ES4N, ES4P, ES45, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D. In some embodiments, the synthetic receptor comprises a SIRP IgV monomer, wherein the SIRP IgV monomer comprises a mutation of Q52H and one additional substitution selected from the group consisting of K53H, K68H, R69H, E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, E54D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, I76H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D. In some embodiments, the synthetic receptor comprises a SIRP IgV monomer, wherein the SIRP IgV monomer comprises a mutation of K68H and one additional substitution selected from the group consisting of Q52H, K53H, R69H, E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31E/L31E, I31D/L31D, I31Q/L31Q, I31N/L31N, I31H/L31H, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, ES4H, E54D, E54Q, E54N, E54P, E54S, E54G, H56P, H56Y, H56R, H56D, H56E, F57H, F57E, F57D, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D.

In some embodiments, the synthetic receptor comprises a SIRP IgV multimer polypeptide comprising two SIRP IgV monomers, wherein the SIRP IgV monomer comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H. In some embodiments, the synthetic receptor comprises a SIRP IgV multimer polypeptide comprising two SIRP IgV monomers, wherein the SIRP IgV multimer polypeptide comprises an amino acid sequence selected from the group consisting of SEQ ID NO: 82 to 96 and SEQ ID NO: 418 to 425. In some embodiments, the synthetic receptor comprises a SIRP IgV multimer polypeptide comprising three SIRP IgV monomers, wherein the SIRP IgV monomer comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H. In some embodiments, the synthetic receptor comprises a SIRP IgV multimer polypeptide comprising three SIRP IgV monomers, wherein the SIRP IgV multimer polypeptide comprises an amino acid sequence selected from the group consisting of SEQ ID NO: 97 to 109 and SEQ ID NO: 430 to 435. In some embodiments, the synthetic receptor comprises a SIRP IgV multimer polypeptide comprising four SIRP IgV monomers, wherein the SIRP IgV monomer comprises a substitution selected from the group consisting of K53H, R69H, Q52H, K68H, Q52H+K68H, K53H+K68H, R69H+K68H, K53H+Q52H, R69H+Q52H, K53H+K68H+Q52H, and R69H+K68H+Q52H. In some embodiments, the synthetic receptor comprises a SIRP IgV multimer polypeptide comprising four SIRP IgV monomers, wherein the SIRP IgV multimer polypeptide comprises an amino acid sequence selected from the group consisting of SEQ ID NO: 110 to 113. Engineered cell therapy using T cells (including αβ T cells, γδ T cells, double negative T cells, Treg), NK cells, NKT cells, cytokine-induced killer cells (CIK), mucosal-associated invariant T cells (MA1T)), monocytes, macrophages, dendritic cells, B cells, mesenchymal stem cells (MSC) and induced pluripotent stem cells (iPSC) expressing synthetic chimeric antigen receptor (CAR), namely CAR-αβT, CAR-γδ T, CAR-DNT, CAR-Treg, CAR-NK, CAR-NKT, CAR-CIK, CAR-MA1T, CAR-macrophage, CAR-DC, CAR-B, CAR-MSC or CAR-IPSC cells respectively, are well described in the arts such as in the patents WO2005044996, U.S. Pat. No. 7,446,190B2, US20130287748A1, WO2012079000A1, WO2012129514, WO2013123061A1, WO2021151119A1, U.S. Pat. No. 10,918,667B2, US20210161961A1, WO2019140100A1, WO2021108926A1, WO2019178518A1, WO2013040371A3, WO2016049459A1 US20210252053A1, WO2016071513A1, WO2020127513A1 U.S. Pat. No. 11,306,134B2, WO2021248061A1 WO2022051556A1, US20160237407A1, U.S. Pat. No. 11,246,890B2, as well as in journal articles such as (Hossian et al., Mol Ther, 2022, Qin et al., Cancers (Basel), 2021, Hong et al., Cancer Cell, 2020, Wu et al., APL Bioeng, 2022, Guedan et al., Mol Ther Methods Clin Dev, 2019, Gao et al., Annu Rev Chem Biomol Eng, 2022, Schmidt et al., Immunother Adv, 2022, Blederstadt et al., Int J Hematol, 2021), all incorporated herein by reference for all purposes. Additionally, engineered cell therapy using T cells expressing a synthetic T cell receptor, such as an exogenous T cell receptor (e.g. TCR-T cells as described in (Zhao et al., Front Immunol, 2021)), a T cell receptor fusion protein (e.g. TRuC-T as described in the patent WO2016187349A1 and journal article (Baeuerle et al., Nat Commun, 2019)), an antibody-T cell receptor chimeric molecule (e.g. AbTCR-T as described in the patent WO2017070608A1 and journal article (Xu et al., Cell Discov, 2018), (Linl et al., Research Square, 2022), and a STAR-T as described in the patent WO2020029774A1 and journal article (Liu et al., Sci Transl Med, 2021)) are well described in the arts. Engineered cell therapy using T cells expressing a T cell-antigen coupler (e.g. TAC-T as described in the patent U.S. Pat. No. 10,435,453B2 and journal article (Helsen et al., Nat Commun, 2018) are also well described in the arts. All the cited patents and articles are incorporated herein by reference for all purposes.

In some embodiments, a chimeric antigen receptor (CAR) comprises an antigen binding domain comprising a SIRP IgV monomer, a SIRP IgV multimer polypeptide, or a SIRP IgV domain-comprising fusion polypeptide of present disclosure. In further embodiments, the chimeric antigen receptor (CAR) additionally comprises a transmembrane domain, and an intracellular signaling domain, in addition to the antigen binding domain comprising a SIRP IgV domain. In additional embodiments, the chimeric antigen receptor (CAR) optionally comprises an extracellular spacer domain and/or at least one co-stimulatory domain. In certain embodiments, the transmembrane domain of the CAR is derived from transmembrane domain of CD28, CD137, CD4, CD8 and/or CD36. In certain embodiments, the intracellular signaling domain of the CAR is derived from the intracellular signaling domain of CD3ζ. In certain embodiments, the intracellular signaling domain is derived from the intracellular signaling domain of Foy receptors, Megf10, MerTK, Dectin-1, and/or CD147, wherein the CAR comprising the intracellular signaling domain is preferentially expressed in a monocyte, a macrophage, a dendritic cell, or a B cell (see {Sloas, 2021 #835} {Wang, 2022 #836}). In certain embodiments, the co-stimulatory domain of the CAR is derived from the group of proteins comprising CD28, CD137, OX40, CD27, ICOS, GITR, CD40, MyD88, CD86, TLR2, TLR4, TLR6, TLR7, TLR8, TLR9, MerTK, Dectin-1, DAP12, DAP10, 2B4, and combination thereof. In one embodiment, the co-stimulatory domain of the CAR is derived from CD28 or CD137. In one embodiment, the co-stimulatory domain of the CAR is derived from CD40, MyD88, CD86, TLR2, TLR4, TLR6, TLR7, TLR8, TLR9, MerTK and/or Dectin-1, wherein the CAR comprising the costimulatory domain is preferentially expressed in a monocyte, a macrophage, a dendritic cell, or a B cell (see {Sloas, 2021 #835} {Wang, 2022 #836}). In certain embodiments, the extracellular spacer domain of the CAR comprises spacer domain selected from the group exemplarily comprising an Fc fragment of an antibody, a hinge region of an antibody, a CH2 region of an antibody, a CH3 region of an antibody, an extracellular spacer region of CD8, an extracellular spacer region of CD4, an extracellular spacer region of CD28, an extracellular spacer region of 4-1BB, an artificial spacer sequence and combinations thereof. Options of additional transmembrane domain, intracellular signaling domain, co-stimulatory domain and/or spacer domain are described in the arts such as patents WO2005044996, U.S. Pat. No. 7,446,190B2, WO2019067805A1, US20220000917A1 and WO2017075147A1.

Examples of CAR constructs are well described in the arts such as in the patents WO2005044996, U.S. Pat. No. 7,446,190B2, US20130287748A1, WO2012079000A1, and WO2012129514, WO2013123061A1, WO2021151119A1, U.S. Pat. No. 10,869,888B2, US20210077532A1, U.S. Pat. No. 10,918,667B2, US20210161961A1, U.S. Pat. No. 10,869,888B2, US20210077532A1, U.S. Pat. No. 11,198,729B2 WO2016081518A2, WO2020072536A1, WO2019213610A1, US20210015859A1, WO2019126748A1, WO2019140100A1, U.S. Pat. No. 11,198,729B2, WO2016081518A2, WO2020072536A1, WO2021108926A1, WO2019178518A1, WO2019213610A1, US20210015859A1, WO2019126748A1, WO2013040371A3, WO2016049459A1, WO2016071513A1, WO2020127513A1, U.S. Pat. No. 11,306,134B2, WO2021248061A1, US20210252053A1, WO2022051556A1, US20160237407A1, U.S. Pat. No. 11,246,890B2. Notably, CD47-targeting CAR-T comprising a non-pH sensitive CD47-binding scFv domain from humanized B6H12 antibody have been generated and reported effective anti-tumor activity in preclinical studies (see {Golubovskaya, 2017 #895} {La, 2021 #896}). Additionally, bispecific CAR-T cells targeting both CD47 and TAG-72, comprising a non-pH sensitive CD47-binding scFv domain from the B6H12 antibody or Hu5F9 antibody, has also been generated and reported as effective anti-tumor activity in preclinical studies (see {Shu, 2021 #893}). However, defective expansion of such non-pH sensitive CD47-targeting CAR-T comprising a fully functional intracellular signaling domain was encountered due to fratricide among the CD47-expressing CAR-T cells (see {Shu, 2021 #893}). The antigen-binding domain of an existing CAR known in the arts can be replaced with a SIRP IgV monomer, a SIRP IgV multimer polypeptide, or a SIRP IgV domain-comprising fusion polypeptide of present disclosure to generate a new CAR binding to CD47. The antigen-binding domain of an existing CAR known in the arts can also be linked at its N-terminal or C-terminal directly or through a linker to a SIRP IgV monomer, a SIRP IgV multimer polypeptide, or a SIRP IgV domain-comprising fusion polypeptide of present disclosure to add binding to CD47 as a bispecific or multispecific CAR. In certain embodiments, the SIRP IgV domain is linked to the N-terminal or C-terminal of the antigen binding domain of a CAR through a linker, wherein the linker sequence comprises (GGGGS)n, wherein n=1 to 4. In certain embodiments, the CAR is a dimeric CAR comprising two separate chimeric antigen receptor polypeptides that homodimerize on the membrane of a cell. Examples of dimeric CAR are well known in the arts such as described in the articles (Jonnalagadda et al., Mol Ther, 2015) (Thomas et al., PLOS One, 2016, Fujiwara et al., Cells, 2020, Jayaraman et al., EBioMedicine, 2020).

In some embodiments, a modified cell expressing a CAR comprising a SIRP IgV domain of present disclosure, comprises an αβ T cell, a yd T cell, a double negative T cell, a Treg cell, a NK cell, a NKT cell, a cytokine-induced killer cell (CIK), a mucosal-associated invariant T cell (MA1T), a monocyte, a macrophage, a dendritic cell, a B cell, a mesenchymal stem cell (MSC) or an induced pluripotent stem cell (iPSC). In certain embodiments, the modified cell is a T cell or NK cell. In certain embodiments, the modified cell is a macrophage. CAR-macrophage is well described in the arts such as in the patents U.S. Pat. No. 11,306,134B2 and WO2021248061A1. Blockade of CD47 is shown to enhance phagocytosis of CAR-macrophages targeting other non-CD47 antigen (see eLife 7:e36688). A CAR-macrophage comprising a SIRP IgV domain of present disclosure can synergistically both block CD47 and self-activate to enhance phagocytosis of CD47-expressing target cells.

In some embodiments, the antigen binding domain of the CAR comprises in total one SIRP IgV monomer comprising a pH-sensitive mutation of K53H, R69H, Q52H, K68H or Q52H+K68H, or no said pH-sensitive mutation. In certain embodiments, the antigen binding domain of the CAR comprises in total one SIRP IgV monomer comprising an amino acid sequence selected from the group consisting of SEQ ID NO: 3 to 81. In some embodiments, the antigen binding domain of the CAR comprises in total two SIRP IgV monomers comprising a pH-sensitive mutation of a mutation of K53H, R69H, Q52H, K68H or Q52H+K68H, or no said pH-sensitive mutation. In certain embodiments, the antigen binding domain of the CAR comprises in total two SIRP IgV monomers, comprising one SIRP IgV multimer polypeptide comprising two SIRP IgV monomers comprising a pH-sensitive mutation of K53H, R69H, Q52H, K68H or Q52H+K68H, or no said pH-sensitive mutation. In certain embodiments, the SIRP IgV multimer polypeptide comprising two SIRP IgV monomers comprises an amino acid sequence selected from the group consisting of SEQ ID NO: 82 to 96 and SEQ ID NO: 418 to 425. In certain embodiments, the antigen binding domain of the CAR comprises in total two SIRP IgV monomers, wherein the CAR comprises a dimeric antigen binding domain comprising two separate SIRP IgV monomers on two separate polypeptides, wherein the two SIRP IgV monomers comprises an amino acid sequence selected from the group consisting of SEQ ID NO: 3 to 81. In some embodiments, the antigen binding domain of the CAR comprises in total three or four SIRP IgV monomers comprising a mutation of K53H, R69H, Q52H, K68H or Q52H+K68H. In some embodiments, the antigen binding domain of the CAR comprises in total three or four SIRP IgV monomers comprising preferentially a mutation of K53H or R69H. In certain embodiments, the antigen binding domain of the CAR comprises in total three or four SIRP IgV monomers comprising a SIRP IgV multimer polypeptide comprising three or four SIRP IgV monomers. In certain embodiments, the SIRP IgV multimer polypeptide comprising three or four SIRP IgV monomers comprises an amino acid sequence selected from the group consisting of optionally SEQ ID NO: 97 to 113 and SEQ ID NO: 430 to 435. In certain embodiments, the antigen binding domain of the CAR comprises in total four SIRP IgV monomers, wherein the CAR comprises a dimeric antigen binding domain comprising two separate SIRP IgV multimer polypeptides each comprising two SIRP IgV monomers. In certain embodiments, the SIRP IgV multimer polypeptide comprising two SIRP IgV monomers comprises an amino acid sequence selected from the group consisting of optionally SEQ ID NO: 82 to 96 and SEQ ID NO: 418 to 425.

In certain embodiments, the CAR comprising a SIRP IgV domain comprises an amino acid sequence selected from the exemplary group consisting of: SEQ ID NO: 254 to 267.

In some embodiments, a T cell receptor fusion protein (TFP) comprises an antigen binding domain comprising a SIRP IgV monomer, a SIRP IgV multimer polypeptide, or a SIRP IgV domain-comprising fusion polypeptide of present disclosure. In further embodiments, the TCR fusion protein comprises a TCR subunit comprising at least a portion of a TCR extracellular domain sequence, a TCR transmembrane domain and a TCR intracellular domain of a TOR subunit, wherein the SIRP IgV domain is linked directly or through a linker to the N-terminal of the TCR subunit and wherein the TCR fusion protein incorporates into a TCR when expressed in a T cell. In certain embodiments, the TCR subunit is selected from the group consisting of CD3ε, CD3γ, CD3δ, TCRα, TCRβ, TCRγ and TCRδ. In one embodiment, the TCR subunit is preferentially CD3ε, wherein the T cell receptor complex comprises two CD3ε units. In certain embodiments, the SIRP IgV domain is linked to the N-terminal of the TCR subunit through a linker, wherein the linker sequence comprises (GGGGS)n, wherein n=1 to 4.

T cell receptor fusion protein comprising a full length TCR subunit fused with scFv-based antigen binding domain is known in the arts, e.g. as described in the patent WO2016187349A1 and journal article (Baeuerle et al., Nat Commun, 2019). The antigen binding domain of an existing said T cell receptor fusion protein known in the arts can be replaced with a SIRP IgV monomer, a SIRP IgV multimer polypeptide, or a SIRP IgV domain-comprising fusion polypeptide of present disclosure to generate a new T cell receptor fusion protein binding to CD47. The antigen-binding domain of an existing said T cell receptor fusion protein known in the arts can also be linked at its N-terminal or C-terminal directly or through a linker to a SIRP IgV monomer, a SIRP IgV multimer polypeptide, or a SIRP IgV domain-comprising fusion polypeptide of present disclosure to add binding to CD47 as a bispecific or multispecific T cell receptor fusion protein. In certain embodiments, the SIRP IgV domain is linked to the N-terminal or C-terminal of the antigen binding domain of a T cell receptor fusion protein through a linker, wherein the linker sequence comprises (GGGGS)n, wherein n=1 to 4.

In some embodiments, a modified T cell expressing a T cell receptor fusion protein comprising a SIRP IgV domain of present disclosure, comprises an αβ T cell, a γδ T cell, a double negative T cell, a Treg cell, a NKT cell, a cytokine-induced killer cell (CIK), a mucosal-associated invariant T cell (MA1T), or an induced pluripotent stem cell (iPSC).

In certain embodiments, a T cell receptor fusion protein comprises a SIRP IgV domain connected through a linker to the N-terminal of full length CD3ε, CD3γ, CD3δ, TCRα, TCRβ, TCRγ or TCRδ, wherein the SIRP IgV domain comprises one SIRP IgV monomer of present disclosure, comprising a pH-sensitive mutation of K53H, R69H, Q52H, K68H or Q52H+K68H, or no said pH-sensitive mutation. In certain embodiments, a T cell receptor fusion protein comprises a SIRP IgV domain connected through a linker to the N-terminal of full length CD3ε, CD3γ, CD3δ, TCRα, TCRβ, TCRγ or TCRδ, wherein the SIRP IgV domain comprises one SIRP IgV monomer of present disclosure, comprising an amino acid sequence selected from the group consisting of SEQ ID NO: 3 to 81. In certain embodiments, a T cell receptor fusion protein comprises a SIRP IgV domain connected through a linker to the N-terminal of full length CD3ε, CD3γ, CD3δ, TCRα, TCRβ, TCRγ or TCRS, wherein the SIRP IgV domain comprises a SIRP IgV multimer polypeptide of present disclosure comprising two SIRP IgV monomers comprising a pH-sensitive mutation of K53H, R69H, Q52H, K68H or Q52H+K68H, or no said pH-sensitive mutation. In certain embodiments, a T cell receptor fusion protein comprises a SIRP IgV domain connected through a linker to the N-terminal of full length CD3ε, CD3γ, CD3δ, TCRα, TCRβ, TCRγ or TCRδ, wherein the SIRP IgV domain comprises a SIRP IgV multimer polypeptide of present disclosure comprising two SIRP IgV monomers, comprising an amino acid sequence selected from the group consisting of SEQ ID NO: 82 to 96 and SEQ ID NO: 418 to 425. In certain embodiments, a T cell receptor fusion protein comprises a SIRP IgV domain connected through a linker to the N-terminal of full length CD3ε, wherein the SIRP IgV domain comprises a SIRP IgV multimer polypeptide of present disclosure comprising two SIRP IgV monomers comprising preferentially a mutation of K53H or R69H. In certain embodiments, a T cell receptor fusion protein comprises a SIRP IgV domain connected through a linker to the N-terminal of full length CD3γ, CD3δ, TCRα, TCRβ, TCRγ or TCRδ, wherein the SIRP IgV domain comprises a SIRP IgV multimer polypeptide of present disclosure comprising three or four SIRP IgV monomers comprising a mutation of K53H, R69H, Q52H, K68H or Q52H+K68H. In certain embodiments, a T cell receptor fusion protein comprises a SIRP IgV domain connected through a linker to the N-terminal of full length CD3γ, CD3δ, TCRα, TCRβ, TCRγ or TCRδ, wherein the SIRP IgV domain comprises a SIRP IgV multimer polypeptide of present disclosure comprising three or four SIRP IgV monomers comprising an amino acid sequence selected from the group consisting of SEQ ID NO: 97 to 113 and SEQ ID NO: 430 to 435. In certain embodiments, a T cell receptor fusion protein comprises a SIRP IgV domain connected through a linker to the N-terminal of full length CD3γ, CD3δ, TCRα, TCRβ, TCRγ or TCRδ, wherein the SIRP IgV domain comprises a SIRP IgV multimer polypeptide of present disclosure comprising three or four SIRP IgV monomers comprising preferentially a mutation of K53H or R69H. In certain embodiments, a T cell receptor fusion protein comprises a SIRP IgV domain connected through a linker to the N-terminal of both full length TCRα and TCRβ, or both full length of TCRγ and TCRδ, wherein the SIRP IgV domain comprises one SIRP IgV monomer comprising a pH-sensitive mutation of K53H, R69H, Q52H, K68H or Q52H+K68H, or no said pH-sensitive mutation. In certain embodiments, a T cell receptor fusion protein comprises a SIRP IgV domain connected through a linker to the N-terminal of both full length TCRα and TCRβ, or both full length of TCRγ and TCRδ, wherein the SIRP IgV domain comprises a SIRP IgV multimer polypeptide comprising two SIRP IgV monomers comprising a mutation of K53H, R69H, Q52H, K68H or Q52H+K68H. In certain embodiments, a T cell receptor fusion protein comprises a SIRP IgV domain connected through a linker to the N-terminal of both full length TCRα and TCRβ, or both full length of TCRγ and TCRδ, wherein the SIRP IgV domain comprises a SIRP IgV multimer polypeptide comprising two SIRP IgV monomers comprising preferentially a mutation of K53H or R69H.

In certain embodiments, the T cell receptor fusion protein comprising a SIRP IgV domain of present disclosure, comprises an amino acid sequence selected from the exemplary group consisting of: SEQ ID NO: 268 to 275.

T cell receptor fusion protein comprising partial TCR chain wherein the variable domain of the TCRα and TCRβ chain, or the TCRγ and TCRδ chain, is replaced by antibody-derived VH or VL or scFv domain is known in the arts, e.g. as described in the patent WO2020029774A1 and journal article (Liu et al., Sci Transl Med, 2021). The antigen binding domain of an existing said T cell receptor fusion protein known in the arts can be replaced with a SIRP IgV monomer, a SIRP IgV multimer polypeptide, or a SIRP IgV domain-comprising fusion polypeptide of present disclosure to generate a new T cell receptor fusion protein binding to CD47. The antigen-binding domain of an existing said T cell receptor fusion protein known in the arts can also be linked at its N-terminal or C-terminal directly or through a linker to a SIRP IgV monomer, a SIRP IgV multimer polypeptide, or a SIRP IgV domain-comprising fusion polypeptide of present disclosure to add binding to CD47 as a bispecific or multispecific T cell receptor fusion protein. In certain embodiments, the SIRP IgV domain is linked to the N-terminal or C-terminal of the antigen binding domain of a T cell receptor fusion protein through a linker, wherein the linker sequence comprises (GGGGS)n, wherein n=1 to 4.

In some embodiments, a modified T cell expressing a T cell receptor fusion protein comprising a SIRP IgV domain of present disclosure, comprises an αβ T cell, a γδ T cell, a double negative T cell, a Treg cell, a NKT cell, a cytokine-induced killer cell (CIK), a mucosal-associated invariant T cell (MA1T), or an induced pluripotent stem cell (iPSC).

In certain embodiments, a T cell receptor fusion protein comprises a SIRP IgV domain connected through a linker to the N-terminal of the constant domain of both partial TCRα and TCRβ, or the constant domain of both partial TCRγ and TCRδ, wherein the SIRP IgV domain comprises one SIRP IgV monomer comprising a pH-sensitive mutation of K53H, R69H, Q52H, K68H or Q52H+K68H, or no said pH-sensitive mutation. In certain embodiments, a T cell receptor fusion protein comprises a SIRP IgV domain connected through a linker to the N-terminal of the constant domain of both partial TCRα and TCRβ, or the constant domain of both partial TCRγ and TCRδ, wherein the SIRP IgV domain comprises one SIRP IgV monomer comprising an amino acid sequence selected from the group consisting of SEQ ID NO: 3 to 81. In certain embodiments, a T cell receptor fusion protein comprises a SIRP IgV domain connected through a linker to the N-terminal of the constant domain of both partial TCRα and TCRβ, or the constant domain of both partial TCRγ and TCRδ, wherein the SIRP IgV domain comprises a SIRP IgV multimer polypeptide comprising two SIRP IgV monomers comprising a mutation of K53H, R69H, Q52H, K68H or Q52H+K68H. In certain embodiments, a T cell receptor fusion protein comprises a SIRP IgV domain connected through a linker to the N-terminal of the constant domain of both partial TCRα and TCRβ, or the constant domain of both partial TCRγ and TCRδ, wherein the SIRP IgV domain comprises a SIRP IgV multimer polypeptide comprising two SIRP IgV monomers, comprising an amino acid sequence selected from the group consisting of SEQ ID NO: 82 to 96 and SEQ ID NO: 418 to 425. In certain embodiments, a T cell receptor fusion protein comprises a SIRP IgV domain connected through a linker to the N-terminal of the constant domain of both partial TCRα and TCRβ, or the constant domain of both partial TCRγ and TCRS, wherein the SIRP IgV domain comprises a SIRP IgV multimer polypeptide comprising two SIRP IgV monomers comprising preferentially a mutation of K53H or R69H.

In certain embodiments, the pair of TCRα and TCRβ fusion protein comprising a SIRP IgV domain of present disclosure, comprises an amino acid sequence selected from the exemplary group consisting of: SEQ ID NO: 276x 277, 278x279, 276×279 and 278 x277, wherein “x” symbol indicates a pair of TCRα and TCRβ fusion protein sequence. In some embodiments, a T cell antigen coupler (TAC) comprises an antigen binding domain comprising a SIRP IgV monomer, a SIRP IgV multimer polypeptide, or a SIRP IgV domain-comprising fusion polypeptide of present disclosure. In further embodiments, the T cell antigen coupler (TAC) additionally comprises a second domain binding to a protein associated with the T cell receptor complex and a third domain comprising a T cell receptor signaling domain. In certain embodiments, the second domain of the TAC binds to a CD3 subunit associated with a TCR complex on a T cell. In one embodiment, the second domain of the TAC comprises a seFv derived from the anti-CD3 antibody UCHT1 or a variant thereof. In another embodiment, the second domain comprises a scFv derived from the anti-CD3 antibody OKT3 or a variant thereof. In another embodiment, the second domain of the TAC comprises a scFv derived from the anti-CD3 antibody SP34 or a variant thereof. In certain embodiments, the T cell signaling domain of the third domain of the TAC comprises an intracellular cytosolic domain and a transmembrane domain. In one embodiment, the cytosolic domain is a CD4 cytosolic domain and the transmembrane domain is a CD4 transmembrane domain. In certain embodiments, the T cell signaling domain optionally further comprises a co-stimulatory domain. In additional embodiments, the co-stimulatory domain is derived from the group of proteins comprising CD28, CD137, OX40, CD27, ICOS and GITR, or combinations thereof.

T cell antigen coupler (TAC) construct linking an antigen-binding domain to a TCR binding domain and a T cell signaling domain is known in the arts, as described in the patent U.S. Pat. No. 10,435,453B2 and journal article (Helsen et al., Nat Commun, 2018). The antigen-binding domain of an existing TAC known in the arts can be replaced with a SIRP IgV monomer, a SIRP IgV multimer polypeptide, or a SIRP IgV domain-comprising fusion polypeptide of present disclosure to generate a new TAC binding to CD47. The antigen-binding domain of an existing TAC known in the arts can also be linked at its N-terminal or C-terminal directly or through a linker to a SIRP IgV monomer, a SIRP IgV multimer polypeptide, or a SIRP IgV domain-comprising fusion polypeptide of present disclosure to add binding to CD47 as a bispecific or multispecific TAC. In certain embodiments, the SIRP IgV domain is linked to the N-terminal or C-terminal of the antigen binding domain of a TAC through a linker, wherein the linker sequence comprises (GGGGS)n, wherein n=1 to 4.

In some embodiments, a modified cell expressing a TAC comprising a SIRP IgV domain of present disclosure, comprises an αβ T cell, a γδ T cell, a double negative T cell, a Treg cell, a NKT cell, a cytokine-induced killer cell (CIK), a mucosal-associated invariant T cell (MA1T), or an induced pluripotent stem cell (iPSC).

In some embodiments, the antigen binding domain of the TAC comprises in total one SIRP IgV monomer comprising a pH-sensitive mutation of K53H, R69H, Q52H, K68H or Q52H+K68H, or no said pH-sensitive mutation. In certain embodiments, the antigen binding domain of the CAR comprises in total one SIRP IgV monomer comprising an amino acid sequence selected from the group consisting of SEQ ID NO: 3 to 81. In some embodiments, the antigen binding domain of the TAC comprises in total two SIRP IgV monomers comprising a pH-sensitive mutation of K53H, R69H, Q52H, K68H or Q52H+K68H, or no said pH-sensitive mutation. In certain embodiments, the antigen binding domain of the CAR comprises in total two SIRP IgV monomers, comprising one SIRP IgV multimer polypeptide comprising two SIRP IgV monomers comprising a pH-sensitive mutation of a mutation of K53H, R69H, Q52H, K68H or Q52H+K68H, or no said pH-sensitive mutation. In certain embodiments, the SIRP IgV multimer polypeptide comprising two SIRP IgV monomers comprises an amino acid sequence selected from the group consisting of SEQ ID NO: 82 to 96 and SEQ ID NO: 418 to 425. In certain embodiments, the antigen binding domain of the CAR comprises in total three or four SIRP IgV monomers, comprising a SIRP IgV multimer polypeptide comprising three or four SIRP IgV monomers comprising a mutation of K53H, R69H, Q52H, K68H or Q52H+K68H. In certain embodiments, the SIRP IgV multimer polypeptide comprising three or four SIRP IgV monomers comprises an amino acid sequence selected from the group consisting of SEQ ID NO: 97 to 113 and SEQ ID NO: 430 to 435. In some embodiments, the antigen binding domain of the CAR comprises in total three or four SIRP IgV monomers comprising preferentially a mutation of K53H or R69H.

In certain embodiments, the TAC comprising a SIRP IgV domain of present disclosure, comprises an amino acid sequence selected from the exemplary group consisting of: SEQ ID NO: 280 to 285. In some embodiments, a bispecific CAR, a bispecific T cell receptor fusion protein or a bispecific TAC comprising a SIRP IgV domain of present disclosure and a second antigen binding domain that binds to a non-CD47 antigen, wherein the SIRP IgV domain is linked through a linker to the N-terminal or C-terminal of the second antigen binding domain. In certain embodiments, the second antigen binding domain of the bispecific CAR, bispecific T cell receptor fusion protein or bispecific TAC binds to an antigen selected from the group consisting of CD19, CD20, CD22, CD37, BCMA, GPRC5D, CD70, NKG2D ligands, ROR1, MSLN, claudin 18.2, claudin 6, GPC3, HER2, GUCY2C, PAP, TSHR, ALPP, GPC3, EGFR-VIII, GD2, DLL3, IL13Ra2, PSMA, PSCA, MUC1, MUC16, FcRa, CD44v6, Necint-4, CAIX, CEA, B7-H3, HPV16-E6, HPV16-E7, AFP, NY-ESO-1, MAGEA4, MAGEA3, MAGEA8, PRAME, COL6A3 and WT1. In one embodiment, the second antigen binding domain of the bispecific CAR, bispecific T cell receptor fusion protein or bispecific TAC binds to CD19 or BCMA. Examples of bispecific CAR are well described in the arts such as in the patents WO2013123061A1 and US20210077532A1 and the articles (Zah et al., Cancer Immunol Res, 2016, Schneider et al., J Immunother Cancer, 2017).

In some embodiments, a modified cell comprises a CAR, a T cell receptor fusion protein or a TAC comprising a SIRP IgV domain of present disclosure, wherein the same cell also comprises a second CAR, a second T cell receptor fusion protein or a second TAC targeting against a non-CD47 antigen. In some embodiments, a modified cell comprise a CAR, a T cell receptor fusion protein or a TAC comprising a SIRP IgV domain of present disclosure, wherein the same cell also comprises a second CAR, a second T cell receptor fusion protein or a second TAC targeting against CD19 or BCMA. Examples of such compound CAR-T cells are well described in the arts such as in the patent WO2019140100A1 and the articles (Petrov et al., Leukemia, 2018, Yan et al., Stem Cell Rev Rep, 2020).

In some embodiments, a population of modified cells comprise a CAR, a T cell receptor fusion protein or a TAC comprising a SIRP IgV domain of present disclosure, wherein the same population of cells and/or a second population of cells comprises a second CAR, a second T cell receptor fusion protein or a second TAC targeting against a non-CD47 antigen. In some embodiments, a population of cells comprise a CAR, a T cell receptor fusion protein or a TAC comprising a SIRP IgV domain of present disclosure, wherein the same population of cells and/or a second population of cells comprises a second CAR, a second T cell receptor fusion protein or a second TAC targeting against CD19 or BCMA. Examples of such coupled CAR-T cells are well described in the arts such as in the patents U.S. Pat. No. 10,869,888B2, US20220265708A1, US20220096546A1 and WO2020146743A1.

In some embodiments, a modified cell comprises a polynucleotide encoding a CAR, a T cell receptor fusion protein or a TAC comprising a SIRP IgV domain of present disclosure, wherein the cell comprises a second polynucleotide encoding a therapeutic agent, wherein the modified cell expresses and secretes the therapeutic agent in response to activation of the modified cell. In some embodiments, a modified cell comprises a polynucleotide encoding a CAR, a T cell receptor fusion protein or a TAC comprising a SIRP IgV domain of present disclosure, and a polynucleotide encoding a second CAR, a second T cell receptor fusion protein or a second TAC that targets against a non-CD47 antigen, wherein the cell comprises another polynucleotide encoding a therapeutic agent, wherein the modified cell expresses and secretes the therapeutic agent in response to activation of the modified cell. In some embodiments, a population of modified cells comprise a polynucleotide encoding a CAR, a T cell receptor fusion protein or a TAC comprising a SIRP IgV domain of present disclosure, wherein the same population of modified cells and/or a different population of modified cells comprise a second polynucleotide encoding a therapeutic agent, wherein the modified cells express and secrete the therapeutic agent in response to activation of the modified cells. In some embodiments, a population of modified cells comprise a polynucleotide encoding a CAR, a T cell receptor fusion protein or a TAC comprising a SIRP IgV domain of present disclosure, wherein the same population of modified cells and/or a different population of modified cells comprise a second polynucleotide encoding a second CAR, a second T cell receptor fusion protein or a second TAC that targets against a non-CD47 antigen, and/or a third polynucleotide encoding a therapeutic agent, wherein the modified cells express and secrete the therapeutic agent in response to activation of the modified cells. In certain embodiments, the non-CD47 antigen targeted by the second CAR, T cell receptor fusion protein or TAC is CD19 or BCMA. In certain embodiments, the polynucleotide encoding the therapeutic agent comprises a NEAT binding sequence in the promoter sequence regulating the expression of the therapeutic agent. In certain embodiments, the NEAT promoter sequence of the polynucleotide encoding the therapeutic agent comprises a nucleotide sequence of SEQ ID NO: 287. In certain embodiments, the therapeutic agent encoded by the polynucleotide comprises a cytokine. In certain embodiments, the cytokine therapeutic agent comprises one or more cytokine selected from the group consisting of IL-6, IFN-γ, IL-12, IL-7, IL-15, and TNFα. In certain embodiments, the cytokine therapeutic agent comprises IL-6 and/or IFN-γ. In certain embodiments, the polynucleotide encoding therapeutic agents IL-6 and/or IFN-γ.

In certain embodiments, the cytokine therapeutic agent comprises IL-12, comprising an amino acid sequence of SEQ ID NO: 288. In certain embodiments, the therapeutic cytokine agent comprises IL-6, IFN-γ and/or IL-12. In certain embodiments, a composition comprises a first population of modified T cells comprising a CAR comprising a SIRP IgV domain of present disclosure, and comprises a second population of modified T cells comprising a CAR against CD19 (SEQ ID NO: 286) and a polynucleotide comprising SEQ ID NO: 290 encoding IL-6 and IFN-γ (encoding a polypeptide of IL-6-P2A-IFN-γ with amino acid sequence of SEQ ID NO: 289) driven by a NFAT promoter (SEQ ID NO: 287), wherein the second population of modified T cells express and secrete IL-6 and IFN-γ in response to activation of their CAR by CD19 antigen. In certain embodiments, a composition comprises a first population of modified T cells comprising a CAR comprising a SIRP IgV domain of present disclosure, and comprises a second population of modified T cells comprising a CAR against CD19 and a polynucleotide encoding IL-12 (with amino acid sequence of SEQ ID NO: 288) driven by a NEAT promoter (SEQ ID NO: 287), wherein the second population of modified T cells express and secrete IL-12 in response to activation of their CAR by CD19 antigen. In certain embodiments, a composition comprises a first population of modified T cells comprising a CAR comprising a SIRP IgV domain of present disclosure, and comprises a second population of modified T cells comprising a CAR against CD19 and a polynucleotide encoding IL-6, IFN-γ and/or IL-12 driven by a NEAT promoter, wherein the second population of modified T cells express and secrete IL-6, IFN-γ and/or IL-12 in response to activation of their CAR by CD19 antigen. Examples of such composition of coupled CAR-T cells expressing and secreting cytokines in response to activation of the CAR-T cells are known in the arts, such as described in the patents in the patents U.S. Pat. No. 10,918,667B2, US20210161961A1, U.S. Pat. No. 10,869,888B2, US20220265708A1, WO2020146743A1 and US20220096546A1.

### Nucleic Acid, Vector, and Host Cell for Synthetic Receptors

In some embodiments, a nucleic acid or nucleic acids comprise a sequence encoding a CAR, a TCR fusion protein, a TAC or other synthetic receptor of present disclosure. In additional embodiments, the nucleic acid is a DNA or a RNA.

In some embodiments, an expression vector comprises a nucleic acid or nucleic acids comprising a sequence encoding a CAR, a TCR fusion protein, a TAC or other synthetic receptor of present disclosure. In additional embodiments, the expression vector is selected from the group consisting of lentivirus vectors, gamma retrovirus vectors, foamy virus vectors, adeno associated virus vectors, adenovirus vectors, pox virus vectors, herpes virus vectors, engineered hybrid viruses, and transposon mediated vectors.

In some embodiments, a cell comprises a CAR, a TCR fusion protein, a TAC or other synthetic receptor of present disclosure, and/or a nucleic acid or nucleic acids comprising a sequence encoding a CAR, a TCR fusion protein, a TAC or other synthetic receptor of present disclosure. In some embodiments, the cell additionally comprises a nucleic acid sequence and/or a vector encoding a cytokine. In certain embodiments, the cell comprises a T cell, NK cell, NKT cell, cytokine-induced killer (CIK) cell, mucosal-associated invariant T (MA1T) cell, monocyte, macrophage, dendritic cell, B cell, granulocyte, neutrophil, innate lymphoid cell (ILC), mesenchymal stem cell (MSC) or induced pluripotent stem cell (iPSC). In certain embodiments, the cell comprises a T cell, NK cell, NKT cell, CIK cell, macrophage, B cell, or iPSC. In further embodiment, the T cell comprises αβ T cell, γδ T cell, double negative T cell and/or Treg cell. In certain embodiments, the cell is an autologous or allogeneic cell.

In some embodiments, a pharmaceutical composition comprises a nucleic acid, a vector and/or a cell comprising a nucleic acid sequence encoding a CAR, a TCR fusion protein, a TAC or other synthetic receptor of present disclosure, and a pharmaceutically acceptable carrier.

In some embodiments, a method of treating a CD47-expressing disease in a mammal comprises administering an effective amount of a nucleic acid, a vector and/or a cell comprising a nucleic acid sequence encoding a CAR, a TCR fusion protein, a TAC or other synthetic receptor of present disclosure, to a mammal in need thereof.

In some embodiments, a method of treating a CD47-expressing disease in a mammal comprising administrating an effective amount of T cells comprising a synthetic antigen-binding receptor (SAR) comprising a CD47-binding SIRP IgV domain, wherein the CD47-binding T cells with the synthetic antigen-binding receptor avoids or reduces fratricide of CD47-positive T cells at physiological pH, wherein the SIRP IgV domain of the T cells with the synthetic antigen-binding receptor comprises a mutation of R69H, K53H, Q52H and/or K68H.

In some embodiments, a method of treating a CD47-expressing disease in a mammal comprising administrating an effective amount of T cells comprising a chimeric antigen receptor (CAR) comprising a CD47-binding SIRP IgV domain, wherein the CD47-binding CAR T cells avoids or reduces fratricide of CD47-positive CAR T cells at physiological pH, wherein the SIRP IgV domain of the CAR T cells comprises a mutation of R69H. In some embodiments, a method of treating a CD47-expressing disease in a mammal comprising administrating an effective amount of T cells comprising a chimeric antigen receptor (CAR) comprising a CD47-binding SIRP IgV domain, wherein the CD47-binding CAR T cells avoids or reduces fratricide of CD47-positive CAR T cells at physiological pH, wherein the SIRP IgV domain of the CAR T cells comprises a mutation of K53H. In some embodiments, a method of treating a CD47-expressing disease in a mammal comprising administrating an effective amount of T cells comprising a chimeric antigen receptor comprising a CD47-binding SIRP IgV domain, wherein the CD47-binding CAR T cells avoids or reduces fratricide of CD47-positive CAR T cells at physiological pH, wherein the SIRP IgV domain of the CAR T cells comprises a mutation of Q52H. In some embodiments, a method of treating a CD47-expressing disease in a mammal comprising administrating an effective amount of T cells comprising a chimeric antigen receptor (CAR) comprising a CD47-binding SIRP IgV domain, wherein the CD47-binding CAR T cells avoids or reduces fratricide of CD47-positive CAR T cells at physiological pH, wherein the SIRP IgV domain of the CAR T cells comprises a mutation of K68H.

In some embodiments, a method of treating a CD47-expressing disease in a mammal comprising administrating an effective amount of T cells comprising a synthetic T cell receptor (TCR) comprising a CD47-binding SIRP IgV domain, wherein the CD47-binding T cells with the synthetic TCR avoids or reduces fratricide of CD47-positive T cells at physiological pH, wherein the SIRP IgV domain of the synthetic TCR comprises a mutation of R69H. In some embodiments, a method of treating a CD47-expressing disease in a mammal comprising administrating an effective amount of T cells comprising a synthetic TCR comprising a CD47-binding SIRP IgV domain, wherein the CD47-binding T cells with the synthetic TCR avoids or reduces fratricide of CD47-positive T cells at physiological pH, wherein the SIRP IgV domain of the synthetic TCR comprises a mutation of K53H. In some embodiments, a method of treating a CD47-expressing disease in a mammal comprising administrating an effective amount of T cells comprising a synthetic TCR comprising a CD47-binding SIRP IgV domain, wherein the CD47-binding T cells with the synthetic TCR avoids or reduces fratricide of CD47-positive T cells at physiological pH, wherein the SIRP IgV domain of the synthetic TCR comprises a mutation of Q52H. In some embodiments, a method of treating a CD47-expressing disease in a mammal comprising administrating an effective amount of T cells comprising a synthetic TCR comprising a CD47-binding SIRP IgV domain, wherein the CD47-binding T cells with the synthetic TCR avoids or reduces fratricide of CD47-positive T cells at physiological pH, wherein the SIRP IgV domain of the synthetic TCR comprises a mutation of K68H.

In some embodiments, a method of treating a CD47-expressing disease in a mammal comprising administrating an effective amount of T cells comprising a T cell antigen coupler (TAC) comprising a CD47-binding SIRP IgV domain, wherein the CD47-binding TAC T cells avoids or reduces fratricide of CD47-positive TAC T cells at physiological pH, wherein the SIRP IgV domain of the TAC T cells comprises a mutation of R69H. In some embodiments, a method of treating a CD47-expressing disease in a mammal comprising administrating an effective amount of T cells comprising a T cell antigen coupler (TAC) comprising a CD47-binding SIRP IgV domain, wherein the CD47-binding TAC T cells avoids or reduces fratricide of CD47-positive TAC T cells at physiological pH, wherein the SIRP IgV domain of the TAC T cells comprises a mutation of K53H. In some embodiments, a method of treating a CD47-expressing disease in a mammal comprising administrating an effective amount of T cells comprising a T cell antigen coupler (TAC) comprising a CD47-binding SIRP IgV domain, wherein the CD47-binding TAC T cells avoids or reduces fratricide of CD47-positive TAC T cells at physiological pH, wherein the SIRP IgV domain of the TAC T cells comprises a mutation of Q52H. In some embodiments, a method of treating a CD47-expressing disease in a mammal comprising administrating an effective amount of T cells comprising a T cell antigen coupler (TAC) comprising a CD47-binding SIRP IgV domain, wherein the CD47-binding TAC T cells avoids or reduces fratricide of CD47-positive TAC T cells at physiological pH, wherein the SIRP IgV domain of the TAC T cells comprises a mutation of K68H.

In some embodiments, the disease is a disease of cancer, fibrosis, atherosclerosis, inflammation or senescence. In certain embodiments, the disease is a disease of cancer, wherein the cancer is ovarian cancer, endometrial cancer, uterine cancer, cervical cancer, breast cancer, gastric cancer, esophageal cancer, lung cancer, head and neck cancer, colorectal cancer, liver cancer, bone cancer, sarcoma, osteosarcoma, brain cancer, multiple myeloma, acute myeloid leukemia (AML), myelodysplastic syndrome, adrenal cancer, anal cancer, bile duct cancer, bladder cancer, basal cell skin cancer, chondrosarcoma, Ewing's sarcoma, eye cancer, gallbladder cancer, gastrointestinal carcinoid tumors, gastrointestinal stromal tumor (GIST), glioma, glioblastoma, hepatocellular cancer, kaposi sarcoma, kidney cancer, laryngeal and hypopharyngeal cancer, leiomyosarcoma, non-small cell lung cancer (NSCLC), small cell lung cancer (SCLC), lung carcinoid tumor, mesothelioma, Merkel cell carcinoma, melanoma, nasal cavity and paranasal sinus cancer, nasopharyngeal cancer, neuroendocrine cancer, neuroblastoma, oral cavity and oropharyngeal cancer, pancreatic cancer, penile cancer, pituitary tumors, prostate cancer, renal cancer, retinoblastoma, rhabdomyosarcoma, salivary gland cancer, squamous cell skin cancer, small intestine cancer, stomach cancer, testicular cancer, thymus cancer, thyroid cancer, uterine sarcoma, urinary tract cancer, vaginal cancer, vulvar cancer, Wilms' tumor, diffuse large β-cell lymphoma (DLBCL), follicular lymphoma, hairy-cell leukemia, Hodgkin's lymphoma, leukemia, acute lymphocytic leukemia (ALL), chronic lymphocytic leukemia (CLL), chronic myeloid leukemia (CML), chronic myelomonocytic leukemia (CMML), lymphoma, Mantle cell lymphoma (MCL), marginal zone lymphoma (MZL), myeloma, Non-Hodgkin lymphoma (NHL), plasmacytoma, or Waldenstrom macroglobulinemia. In certain embodiments of the method, the mammal is a human.

### Combination Therapy

In one aspect, the present disclosure provides a method of combination therapy in human comprising administering a therapeutically effective amount of a SIRP IgV monomer of present disclosure, a SIRP IgV multimer polypeptide of present disclosure, a fusion polypeptide of present disclosure, a fusion protein of present disclosure, a protein drug conjugate of present disclosure, and/or a cell therapy comprising a synthetic receptor of present disclosure, and a therapeutically effective amount of another therapy. In some embodiments, another therapy comprises administration of one or more of chemotherapy, molecular targeted therapy, biologics therapy, immunotherapy and radiotherapy. In some embodiments, another therapy comprises a chemotherapy, molecular targeted therapy, biologics therapy, immunotherapy and/or radiotherapy that enhance pro-phagocytic signal and/or inhibit anti-phagocytic signal. In some embodiments, another therapy comprises a chemotherapy, molecular targeted therapy, biologics therapy, immunotherapy and/or radiotherapy that comprise cell adhesion among diseased cells and/or between diseased cells and extracellular matrix. In some embodiments, another therapy comprises a molecular targeted therapy targeting CD117. In some embodiments, another therapy comprises a molecular targeted therapy targeting CD117 selected from the group consisting of CDX-0158, CDX-0159, briquilimab (JSP191), eglatoprutug and MGTA-117.

### Examples

The following examples are included to further describe some embodiments of the present disclosure. The examples are illustrative, but not limiting the scope of the disclosure. Other suitable modifications and adaptations of the variety of conditions and parameters normally encountered in the field, and which are obvious to those skilled in the art, are within the scope of the disclosure.

### General Methods

### 1) Construction of the Plasmids

The DNA encoding the protein sequences is optimized for expression in Homo sapiens, synthesized and cloned into the pcDNA3.4 mammalian expression vector using standard molecular cloning techniques. For proteins without Fe, the protein polypeptide is cloned into a pcDNA3.4 expression vector with a 6× His-tag added to the C-terminal of the protein polypeptide for purification through Ni-NTA column.

### 2). Protein Expression and Purification

The plasmids encoding the polypeptides of a given protein is co-transfected into HEK293 cells usually using polyethylenimine (PEI). For antibody proteins with heterodimeric knobs-into-holes Fe, a 1:1 ratio of knob: hole heavy chain and a 3:2 ratio of light chain: heavy chain are usually used for the plasmids co-transfection. HEK293 cells are grown in a humidified incubator at 37° C. with 8% CO₂ for 5-7 days after transfection. The cells are pelleted by centrifugation at 8000 rpm for 5 minutes and the supernatant is filtered through a 0.2 μm membrane. Pre-assembled Protein A resin column or Ni-NTA resin column is first equilibrated with 1×PBS and the cell supernatant is then loaded through the column. Protein A resin column is washed with 1×PBS and Ni-NTA resin column is washed with 2.5 mM imidazole buffer. The bound protein is then eluted from the column with acidic citrate elution buffer (pH 3.4) for Protein A resin column or 250 mM imidazole buffer for Ni-NTA resin column. The collected proteins is dialyzed into 1×PBS.

### 3) ELISA Assay

ELISA assays are performed using 96-well ELISA plate coated with the capture protein in 1×PBS/well at 4° C. overnight. After washing with 1×PBS+0.05% Tween 20 (PBST), the plate is blocked with 1×PBS+5% milk for 2 hours at room temperature (RT). After washing the plate with 1×PBST, serially diluted concentrations of the analyte in 1×PBS+1% milk are added and incubated for 60 minutes at RT. After washing with 1×PBST, 1:4000 diluted anti-His-HRP (Proteintech, CAT #HRP-66005) or anti-human IgG Fc-HRP (Abcam, CAT #ab97225) in 1×PBS+1% milk is added and incubated for 60 minutes at RT. After washing with 1×PBST, 30 μl/well tetramethylbenzidine (TMB) substrate solution is added and incubated in dark at room temperature for several minutes. 30 μl/well H25O4 is then added to stop the reaction, and OD450 is read out at 450 nm using a microplate reader and data are analyzed by Graphpad Prism 9.

### 4). FACS Assay

Target cells of interest (e.g. Raji cells) are grown into proper confluence and then harvested according to standard procedures. The harvested cells are centrifuged and the cell pellet is resuspended into proper concentration in the cell culture media and incubated at 37° C. for 30 minutes. Serially diluted test article is then added into the cells to designated final concentration and incubated at 4° C. for 30 minutes. The cells are then washed for three times, and fluorescence labeled secondary antibody (anti-human IgG Fc, Abcam #,ab98596, 1:300) is added into the cells and incubated at 4° C. for 30 min. After wash, the cells are resuspended in FACS buffer and analyzed by Flow Cytometry.

### Example 1. Generation of SIRPαV2 Variants for Higher Binding to CD47 at an Acidic pH than at Physiological pH

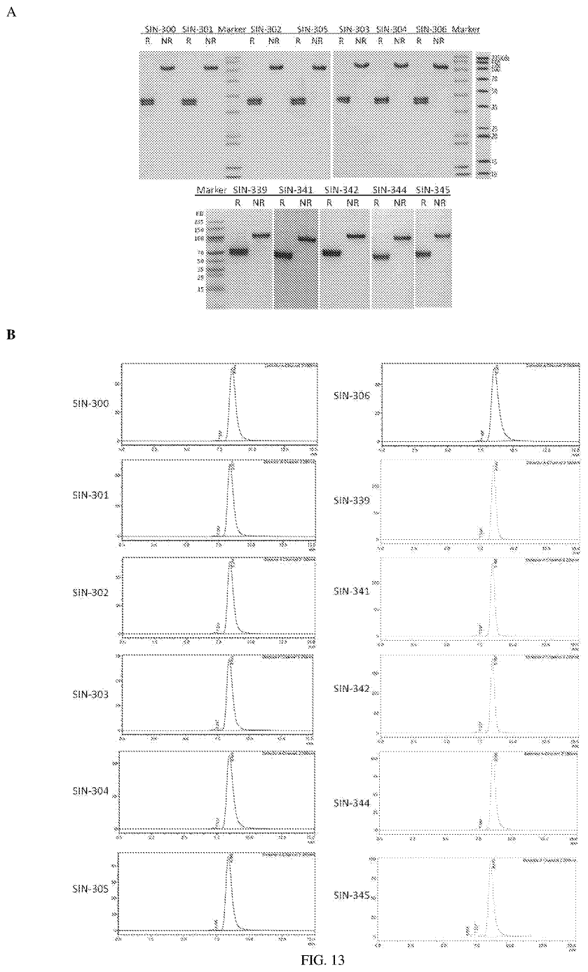

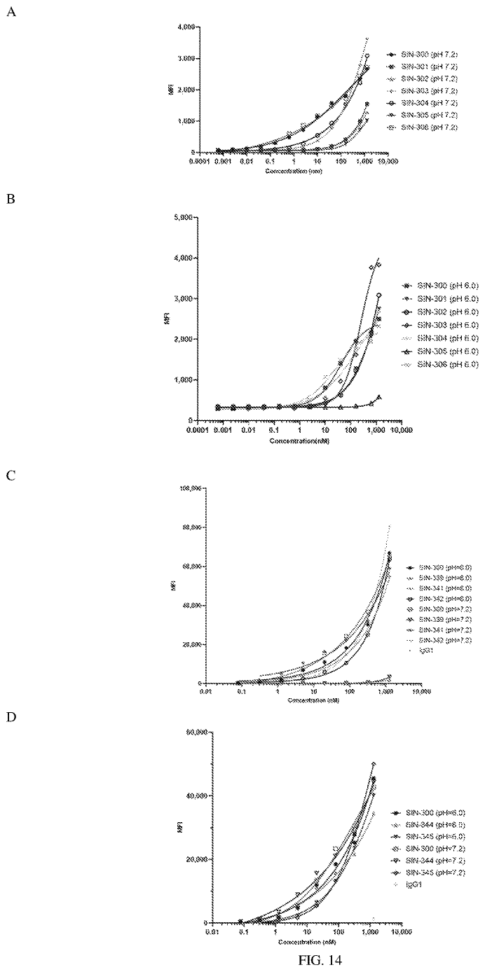

The IgV extracellular domain of wild type human SIRPαV2 (SIRPαV2 IgV WT domain, SEQ ID NO: 4) was first selected as the template for generating variants with differential binding to human CD47 (NCBI accession #: NP_001768.1) at an acidic pH than at physiological pH. Insight gained through proprietary analysis of the amino acid sequence, crystal structure and interaction of SIRPα and CD47 was used to identify residues of wild type SIRPαV2 IgV (SIRPαV2 IgV WT) for histidine substitution to generate potential pH-sensitive binding to CD47. Eleven initial candidate residues of SIRPαV2 IgV WT were selected, including position V33, Q37, N51, Q52, K53, K68, R69, E70, M72, K96 and G97 of SEQ ID NO: 4, for generating single histidine substitution with predicted higher binding to CD47 at an acidic pH than at physiological pH. DNA constructs encoding fusion protein of wild type SIRPαV2 IgV (SEQ ID NO: 4) or SEQ ID NO:4 derived variant SIRPαV2 IgV with single histidine substitution of V33H, Q37H, N51H, Q52H, K53H, K68H, R69H, E70H, M72H, K96H or G97H directly fused to the N-terminal of human IgG1 Fc (SEQ ID NO: 135) were generated through gene synthesis and cloned into pcDNA3.4 mammalian expression vector. The SIRPαV2 IgV-IgG1 Fe fusion proteins were then produced through transient expression of the vector in HEK293 cells and single step of Protein A chromatography purification. The proteins were analyzed by SDS-PAGE and SEC-HPLC. As shown in FIG. 13 and Table 6 below, all the variant SIRPαV2 IgV-IgG1 Fc fusions were expressed and purified with high yield and purity as good as the wild type SIRPαV2 IgV-IgG1 Fe protein, through a single step Protein A chromatography purification. Two close bands seen in reducing SDS-PAGE results for all the proteins were due to known glycosylation effect of SIRPαV2 IgV domain (see U.S. Pat. No. 10,800,821B2).

TABLE 6
Transient expression of wild type and variant SIRPαV2 IgV-IgGI Fc fusion proteins.

| | SIN- | SIN- | SIN- | SIN- | SIN- | SIN- | SIN- | SIN- | SIN- | SIN- | SIN- | SIN- |
|---|---|---|---|---|---|---|---|---|---|---|---|---|
| Protein name | 300 | 301 | 302 | 303 | 304 | 305 | 306 | 339 | 341 | 342 | 344 | 345 |
| Mutation on | None | K53H | R69H | Q52H | K68H | K96H | M72H | V33H | Q37H | N51H | E70H | G97H |
| SIRPαV2 IgV WT | | | | | | | | | | | | |
| template (SEQ ID | | | | | | | | | | | | |
| WT or Variant | 300 | 301 | 302 | 303 | 304 | 305 | 306 | 339 | 341 | 342 | 344 | 345 |
| SIRPαV2 IgV-IgG1 | | | | | | | | | | | | |
| Fc polypeptide SEQ | | | | | | | | | | | | |
| Yield (mg/L) | 122.8 | 106.4 | 122 | 109.6 | 87.2 | 134.4 | 144.4 | 82 | 111 | 136.3 | 177.3 | 168.7 |
| Molecular weight | 80 KD | 80 KD | 80 KD | 80 KD | 80 KD | 80 KD | 80 KD | 80 KD | 80 KD | 80 KD | 80 KD | 80 KD |
| (through SDS- | | | | | | | | | | | | |
| SDS-PAGE purity | >95% | >95% | >95% | >95% | >95% | >95% | >95% | >95% | >95% | >95% | >95% | >95% |
| SEC-HPLC purity | 99.6% | 99.4% | 98.7% | 99.0% | 99.1% | 98.8% | 99.2% | 98.7% | 99.0% | 98.9% | 98.9% | 96.8% |

### Example 2. Binding Activities of Variant SIRPαV2 IgV-IgG1 Fc Fusion Proteins Measured by FACS

The binding activities of the SIRPαV2 IgV-IgG1 Fe fusion proteins from Example 1 to Raji cancer cells were measured by fluorescence activated cell sorting (FACS) at pH 7.2 which represents the lower boundary of physiological pH range (pH 7.2 to 7.5) and at an acidic pH 6.0. Serially diluted SIRPαV2 IgV-IgG1 Fe fusion proteins in 1×PBS+2% FBS at pH 7.2 or 6.0 were added at final concentrations of 1280 nM, 640 nM, 160 nM, 40 nM, 10 nM, 2.5 nM, 0.625 nM, 0.15625 DM, 0.0391 nM, 0.0098 nM, 0.0024 nM and 0.0006 nM, and incubated with Raji cells in 96-well plate (2×10⁵/well) at 4° C. for 30 minutes. After washing the cells with 1×PBS+2% FBS at pH 7.2 or 6.0, FITC-labelled anti-human IgG Fc secondary antibody (Abcam #, ab98596, 1:300) were added and incubated at 4° C. for 30 minutes at pH 7.2 or 6.0. After washing, the Raji cells were then analyzed by FACS in 1×PBS+2% FBS at pH 7.2 or 6.0.

The FACS results in FIG. 14A show that the variant SIRPαV2 IgV-IgG1 Fc fusion protein SIN-306 (M72H) showed no change in binding than wild type SIRPαV2 IgV-IgG1 Fc (SIN-300) at pH 7.2. However, the variant protein SIN-301 (K53H), SIN-302 (R69H), SIN-303 (Q52H), SIN-304 (K68H) and SIN-305 (K96H) all showed reduced binding to Raji cells at pH 7.2 than wild type SIRPαV2 IgV-IgG1 Fe fusion protein SIN-300 which has the exact same amino acid sequence as clinically tested TTI-621. SIN-301 (K53H), SIN-302 (R69H) and SIN-305 (K96H) show particularly weak binding at pH 7.2. On the other hand, the results in FIG. 14B show that at pH 6.0, SIN-306 (M72H) showed still no change in binding from wild type SIRPαV2 IgV-IgG1 Fc (SIN-300), whereas SIN-305 (K96H) showed even lower and minimal binding at pH 6.0 than at pH 7.3. However, SIN-301 (K53H), SIN-302 (R69H), SIN-303 (Q52H) and SIN-304 (K68H) all showed enhanced binding to Raji cells at pH 6.0. Meanwhile, the results in FIGS. 14C and D show that: 1) the variant SIRPαV2 IgV-IgG1 Fe fusion protein SIN-339 (V33H) abolished binding at both physiological pH 7.2 and acidic pH 6.0; 2) Similar to SIN-306 (M72H), SIN-341 (Q37H) showed no binding difference from the wild type SIRPαV2 IgV-IgG1 Fc (SIN-300) at both physiological pH 7.2 and acidic pH 6.0. This is unexpected, as a Q37H mutation of SIRPαV2 IgV was reported to mildly increase binding to CD47-high and low cells (see U.S. Pat. No. 11,021,694); 3) SIN-342 (N51H) showed mildly lower binding than wild type SIN-300 at both physiological pH 7.2 and acidic pH 6.0, but also showed similar binding at both both physiological pH 7.2 and acidic pH 6.0 as similar to wild type SIN-300. Together these results of the 11 variants tested identified the histidine substitution K53H, R69H, Q52H or K68H of SIRPαV2 IgV (SEQ ID NO: 4) rendered higher binding to CD47 at an acidic pH than at physiological pH.

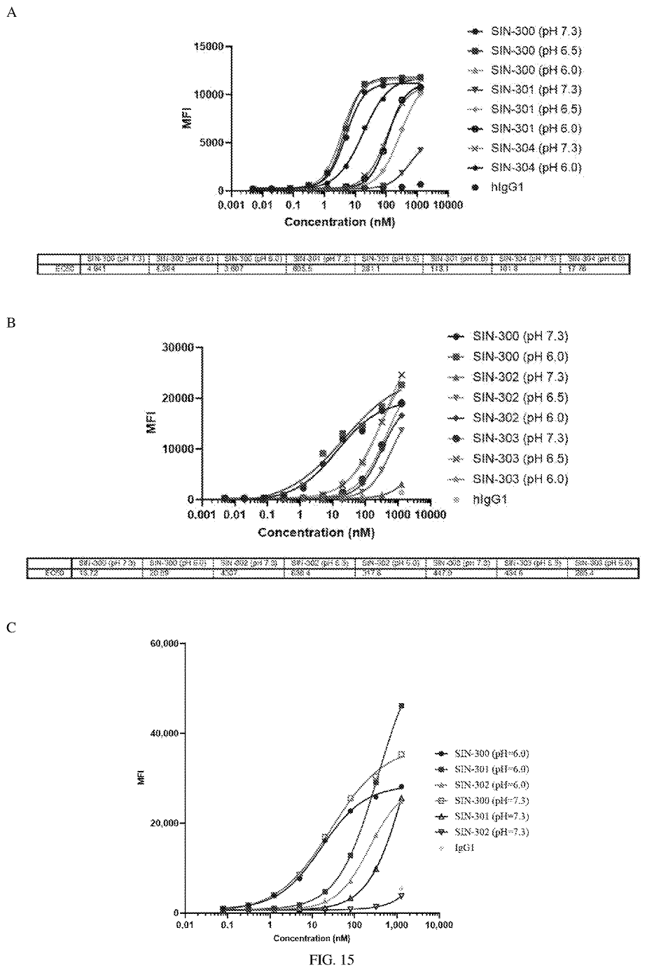

To further characterize the pH-sensitive CD47 binding of SIN-301 (K53H), SIN-302 (R69H), SIN-303 (Q52H) and SIN-304 (K68H), FACS analysis of their binding to Raji cells at pH 7.3, pH 6.5 and pH 6.0 was performed. The results in FIGS. 15A and 15B show that binding of wild type SIRPαV2 IgV-IgG1 Fc (SIN-300) to Raji cells didn't significantly change at the different pH, but the binding to Raji cells increased as the pH decreased from pH 7.3 to pH 6.5 and then to pH 6.0 for SIN-301 (K53H) and SIN-302 (R69H). Additionally, FACS analysis of SIN-301 (K53H) and SIN-302 (R69H) on another cell line SKOV3 expressing lower level of CD47 than Raji (Wang et al., J Immunother Cancer, 2020, Golubovskaya et al., Cancers (Basel), 2017) was performed. The results in FIG. 15C show that, similar to the results in Raji cells, SIN-301 (K53H) and SIN-302 (R69H) also exhibited higher binding to SKOV3 cells at an acidic pH 6.0 than the weak binding at physiological pH 7.3.

### Example 3. Binding Activities of Variant SIRPαV2 IgV-IgG1 Fe Fusion Proteins Measured by ELISA

The binding of the variant SIRPαV2 IgV to CD47 protein was further measured by ELISA at pH 7.2 and at pH 6.0. 96-well ELISA plate was coated with 8 μg/mL SIRPαV2 IgV-IgG1 Fc fusion protein SIN-300 (wild type), SIN-301 (K53H), SIN-302 (R69H), SIN-303 (Q52H) or SIN-304 (K68H) in 30 μl 1×PBS/well at 4° C. overnight. After washing with 1×PBS+0.05% Tween 20 (PBST), the plate was blocked with 1×PBS+5% milk for 2 hours at room temperature (RT). After washing the plate with 1×PBST, 4-fold serially diluted concentrations of His-tagged CD47 extracellular domain (ECD) protein (Abclonal, CAT #RP01306) at 200 nM, 50 nM, 12.5 nM, 3.125 nM, 0.781 nM, 0.195 nM, 0.049 nM and 0.012 nM in 1×PBS+1% milk at pH 7.2 or 6.0, were added (30 μl/well) and incubated for 60 minutes at RT. After washing with 1×PBST, 1:4000 diluted anti-His-HRP (Proteintech, CAT #HRP-66005) in 1×PBS+1% milk at pH 7.2 or 6.0 was added and incubated for 60 minutes at RT. After washing with 1×PBST, 30 μl/well tetramethylbenzidine (TMB) substrate solution was added and incubated in dark at room temperature for several minutes. 30 μl/well 2M H25O4 was then added to stop the reaction, and OD₄₅₀ was read out at 450 nm using a SpectraMax 190 microplate reader and data were analyzed by Graphpad Prism 9. Because monovalent his-tag CD47 ECD protein was used to dected coated SIRPαV2 IgV-IgG1 Fc fusion proteins, the binding reflects monovalent affinity between the SIRPαV2 IgV variants and CD47.

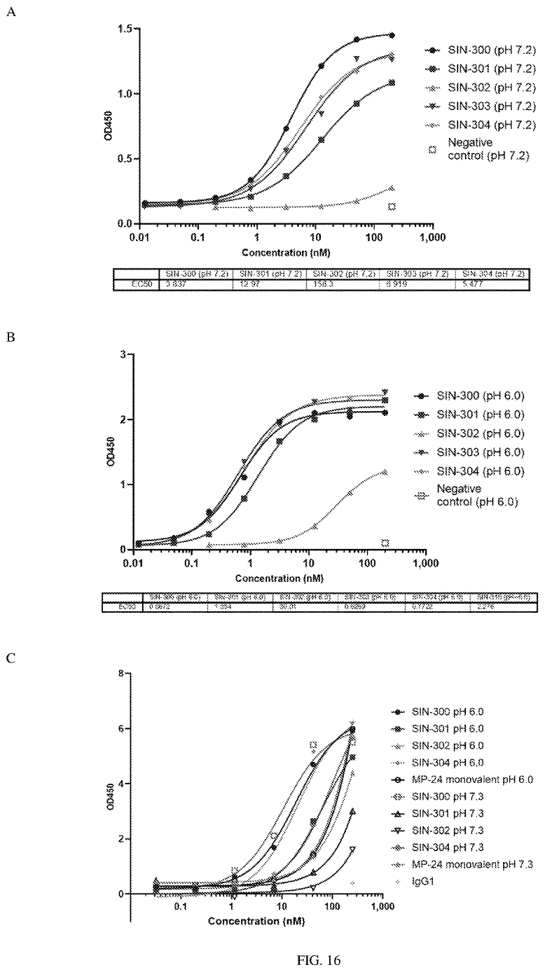

Similar to FACS results in Example 2 above, the ELISA results in FIG. 16A show that SIN-301 (K53H), SIN-302 (R69H), SIN-303 (Q52H) and SIN-304 (K68H) all exhibited reduced binding to CD47 than wild type SIRPαV2 IgV-IgG1 Fc (SIN-300) at pH 7.2. SIN-302 (R69H) particularly showed lowest and minimal binding to CD47 at pH 7.2 and SIN-301 (K53H) showed a large reduction in binding to CD47 at pH 7.2. SIN-303 (Q52H) and SIN-304 (K68H) showed similarly smaller reduction in binding to CD47 at pH 7.2. Meanwhile, the results in FIG. 16B show that SIN-302 (R69H) regained binding to CD47 at pH 6.0 and SIN-301 (K53H) showed increased binding to CD47 that's close to the binding of wild type SIRPαV2 IgV-IgG1 Fc (SIN-300) at pH 6.0. Both SIN-303 (Q52H) and SIN-304 (K68H) showed similar binding to CD47 as that of wild type SIRPαV2 IgV-IgG1 Fc (SIN-300) at pH 6.0.

To benchmark the binding significance of SIN-301 at physiological pH, another ELISA was similarly done, but using the His-tagged CD47 extracellular domain (ECD) protein as immobilized ligand and fusion proteins comprising bivalent or monovalent SIRPαV2 IgV domain as analyte, to also detect bivalent avidity effect of the binding. The results in FIG. 16C show the benchmark protein MP-24 comprising monovalent wild type SIRPαV2 IgV domain exhibited much reduced binding to CD47 than the benchmark bivalent protein SIN-300 (TTI-621 analog) comprising bivalent wild type SIRPαV2 IgV domains. Meanwhile, the CD47 binding of both SIN-301 (bivalent SIRPαV2 IgV K53H variant-IgG1 Fc fusion protein) and SIN-302 (bivalent SIRPαV2 IgV R69H variant-IgG1 Fc fusion protein) were even weaker at physiological pH 7.3 than the benchmark monovalent wild type binding of MP-24 (FIG. 16C). At an acidic pH 6.0, SIN-301 exhibited stronger binding than, while SIN-302 exhibited binding close to, that of the benchmark monovalent wild type binding of MP-24 (FIG. 16C). In contrast, the CD47 binding of SIN-304 (bivalent SIRPαV2 IgV K68H-IgG1 Fe fusion protein) was stronger than the benchmark monovalent binding of MP-24 at physiological pH 7.3 and was further increased close to the level of the benchmark bivalent binding of SIN-300 at an acidic pH 6.0. Monovalent wild type SIRPαV2 IgV domain in tumor targeting bispecific antibody fusion protein configured in similar format as MP-24, is shown to be effective in promoting phagocytosis of target tumor cells with no significant effect on RBCs and platelets and better safety profile than bivalent wild type SIRPαV2 IgV-IgG1 Fe protein (Liu et al., Cell Rep, 2018). Given SIN-301 and SIN-302 showed lower CD47 binding at physiological pH but stronger or similar CD47 binding at an acidic pH than monovalent MP-24, the results in FIG. 16C indicate an even better safety and/or activity profile for bivalent SIRPαV2 IgV domains with K53H or R69H mutation than monovalent wild type SIRPαV2 IgV domain.

Together with the FACS results in Example 2, these results show that the mutation of R69H or K53H largely reduces SIRPαV2 IgV binding to CD47 at physiological pH to much weaker binding than wild type SIRPαV2 IgV, but restores significant binding to CD47 at an acidic pH. Similarly, the mutation of Q52H or K68H also significantly reduces SIRPαV2 IgV binding to CD47 at physiological pH, although to a less extent than R69H or K53H mutation, and restores significant binding to CD47 at an acidic pH that's stronger than the binding of R69H or K53H mutation at an acidic pH. Such a binding profile of SIRP IgV domain with R69H, K53H, Q52H or K68H mutation is desirable to reduce or minimize binding to the ubiquitous CD47-expressing normal tissues and cells, but allow binding to CD47-expressing disease cells in an acidic disease microenvironment, such as CD47-expressing tumor cells in solid tumors.

### Example 4. Binding Activities of Variant SIRPαV2 IgV-IgG1 Fc Fusion Proteins to Human Primary Platelets Measured by FACS

Thrombocytopenia was reported as a most frequent adverse event for both TTI-621 (a wild type SIRPα. V2 IgV-IgG1 Fc fusion protein with the same amino acid sequence of SIN-300) and TTI-622 (a wild type SIRPαV2 IgV-IgG41 Fc fusion protein) in clinical trials and an initial dose limiting toxicity for TTI-621 (Trillium Therapeutics, Inc. R&D Day presentation, Apr. 28, 2021, {Ansell, 2021 #901} and Blood 138 (2021) 2448-2451). These indicate significant binding of wild type SIRPαV2 IgV to platelets despite its minimal binding to red blood cells (RBCs). The binding of the variant SIRPαV2 IgV-IgG1 Fc fusion proteins to platelets at physiological pH 7.3 were thus tested by FACS.

Human primary platelets were seeded into 96-well plate at 1×10⁶/well and incubated with Fc blocking antibody at room temperature. After washing the platelets with 1×PBS+2% FBS pH 7.3, three-fold serially diluted SIRPαV2 IgV-IgG1 Fc fusion proteins in 1×PBS+2% FBS at pH 7.3 were added at final concentrations of 1280 nM, 426.67 nM, 142.22 nM, 47.41 nM, 15.80 nM and 5.27 nM, and incubated with the platelets at 4° C. for 30 minutes. In addition, a magrolimab (Hu5F9) analog protein was generated and used as a benchmark control. Also, a monovalent wild type SIRPαV2 IgV-IgG1 Fe protein (MP-24) and monovalent variant K53H SIRPαV2 IgV-IgG1 Fe protein (MP-25) were generated and used for the FACS binding. After washing the platelets with 1×PBS+2% FBS at pH 7.3, FITC-labelled anti-human IgG Fc secondary antibody (Abcam, CAT #ab6854) were added and incubated at 4° C. for 30 minutes at pH 7.3. After washing, the platelets were then analyzed by FACS in 1×PBS+2% FBS at pH 7.3.

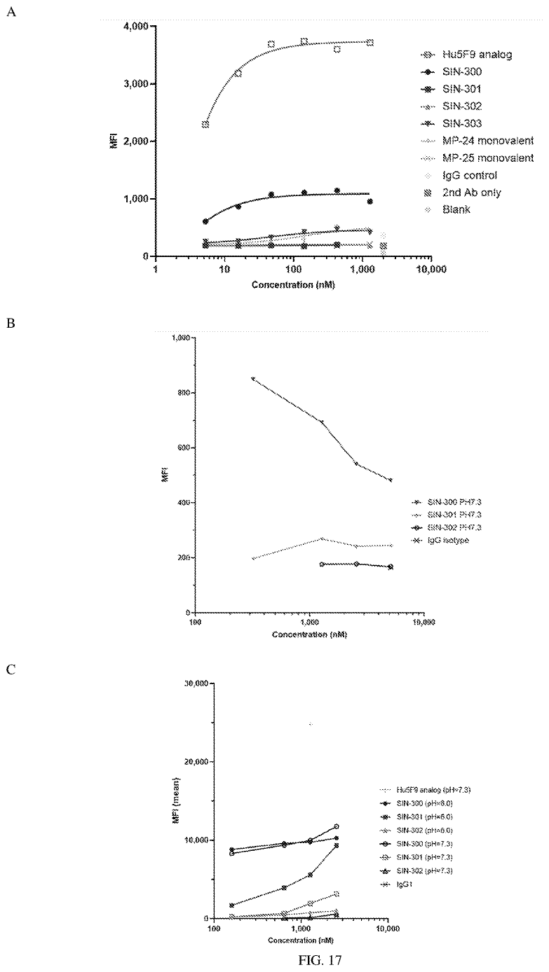

The FACS results in FIG. 17A show that the homodimeric bivalent wild type SIRPαV2 IgV-IgG1 Fc fusion protein SIN-300 exhibited significant binding to platelets that plateaued after 47 nM, but its peak binding MFI to platelets was less than ⅓ of that of the Hu5F9 analog. Meanwhile, the benchmark protein MP-24 comprising monovalent wild type SIRPαV2 IgV domain showed largely reduced binding to platelets than the bivalent SIN-300. Notably, the bivalent variant protein SIN-301 (K53H) and SIN-302 (R69H) as well as the protein MP-25 comprising monovalent SIRPαV2 IgV K53H domain all showed negligible binding to human platelets at pH 7.3. Meanwhile, the bivalent variant SIN-303 (Q52H) showed similar weak binding to platelets as monovalent MP-24 at pH 7.3. In addition, the bivalent variant SIN-302 (R69H) and SIN-301 (K53H) continued to show negligible or very weak binding to human primary platelets at physiological pH 7.3, even at higher concentrations up to 5,125 nM or 410 μg/ml (FIG. 17B) tested, which reaches around the reported Week 1 peak serum concentration of the highest 18.0 mg/kg IV dose of TTI-622 tested in clinical trials (Trillium Therapeutics, Inc. R&D Day presentation, Apr. 28, 2021). These results indicate negligible or very weak platelet binding for the variant protein SIN-302 (R69H) and SIN-301 (K53H) and also weakened platelet binding for the variant protein SIN-303 (Q52H) and SIN-304 (K68H) at physiological pH to avoid or reduce thrombocytopenia in human patients, even at high clinical doses.

### Example 5, Binding Activities of Variant SIRPαV2 IgV-IgG1 Fe Fusion Proteins to Human Primary T Cells Measured by FACS

T cells show high surface CD47 expression among human primary cells with CD47 expression density reported at 83,477 surface CD47 receptors per cell, versus 34,439 per cell for platelets (Puro et al., Mol Cancer Ther, 2020). The binding of the variant SIRPαV2 IgV-IgG1 Fc fusion proteins to human primary T cells at physiological pH 7.3 were thus also tested by FACS.

Human primary T cells were seeded into 96-well plate at 1×10⁵′/well in RPMI-1640 medium at pH 7.3 or 6.0, and incubated at 37° C. for 30 minutes. After washing the T cells, serially diluted SIRPαV2 IgV-IgG1 Fc fusion proteins were added at final concentrations of 2560 nM, 1280 nM, 640 nM and 160 nM at pH 7.3 or 6.0, and incubated with the T cells at 4° C. for 30 minutes. In addition, a magrolimab (Hu5F9) analog protein was used as a benchmark control. After washing the cells with 1×PBS+2% FBS at pH 7.3 or 6.0, PE-labelled anti-human IgG Fc secondary antibody (Abcam, CAT #ab98596) were added and incubated at 4° C. for 30 minutes at pH 7.3. After washing, the T cells were then analyzed by FACS in FACS buffer at pH 7.3 or 6.0.

The FACS results in FIG. 17C show that the homodimeric bivalent wild type SIRPαV2 IgV-IgG1 Fo fusion protein SIN-300 exhibited significant binding to human T cells at both pH 7.3 and 6.0, but its peak binding MFI to human T cells was less than ½ of that of the Hu5F9 analog. Consistent with the binding result in platelets above, the variant protein SIN-302 (R69H) showed negligible binding to human T cells at pH 7.3 even at high concentration of 2,560 nM, whereas the variant protein SIN-301 (K53H) showed mild binding to human T cells at pH 7.3 at high concentrations above 1,000 nM. At an acidic pH 6.0, SIN-302 (R69H) still shows minimal binding to human T cells even at high concentration of 2,560 nM, while SIN-301 (K53H) showed increased binding to T cells at an acidic pH 6.0, reaching similar binding MFI as that of wild type SIN-300 at the high concentration of 2,560 nM. These results indicate negligible or weak T cell binding for variant protein SIN-302 (R69H) and SIN-301 (K53H) at physiological pH, thus allowing SIRP IgV domain with R69H and/or K53H mutation to be used as a binding domain for CD47-targeting T cell engagers or chimeric antigen receptor (CAR) T cell therapy or other engineered T cell therapy that avoid or reduce fratricide of CD47-positive T cells.

### Example 6. Generation and Test of Variant IgV of Other SIRP Family Subtypes for Higher Binding to CD47 at an Acidic pH than at Physiological pH

As shown in FIGS. 1A and 1B, the residues Q52, K53, K68 and R69 of SIRPαV2 IgV (SEQ ID NO: 4) are conserved across all the 10 subtypes of SIRPα(SEQ ID NO: 3 to 11) as well as SIPRβ and SIPRγ. Thus, the same histidine substitution of K53H, R69H, Q52H or K68H can be applied to the IgV of SIRPαV1 (SEQ ID NO: 3), SIRPαV8 (SEQ ID NO: 10), SIPRβ2-D (SEQ ID NO: 17), SIPRγ (SEQ ID NO: 18), SIPRγ-Q (SEQ ID NO: 19), SIPRγ-D (SEQ ID NO: 20) and SIPRγ-QD (SEQ ID NO: 21) as templates to generate variant SIRP IgV on each of these templates respectively. Directly fusing these variant SIRP IgV domains to the N-terminal of human IgG1 Fc (SEQ ID NO: 135) leads to generation of the SIRP IgV-IgG1 Fc fusion proteins as listed in Table 7 below, similarly as the generation of variant SIRPαV2-IgG1 Fc fusion proteins detailed in Example 1. It's also noted that among the 10 SIRPα subtypes, SIRPαV5, SIRPαV6 and SIRPαV9 each differ from SIRPαV1 in only one residue outside the CD47-binding area, SIRPαV3, SIRPαV7 and SIRPαV10 each differ from SIRPα. V2 in only one residue, while SIRPαV4 and SIRPαV8 both comprising a mixture of the 13 amino acid differing between human SIRPαV1 and SIRPαV2 (FIG. 1A). Importantly, the differing residues among the SIRPα subtypes locate outside their CD47-binding area and do not affect binding to CD47, as described in (Hatherley et al., J Biol Chem, 2014). Thus, the variant examples of SIRPαV1, SIRPαV2 and SIRPαV8 tested shall represent all the other SIRPαsubtypes as well.

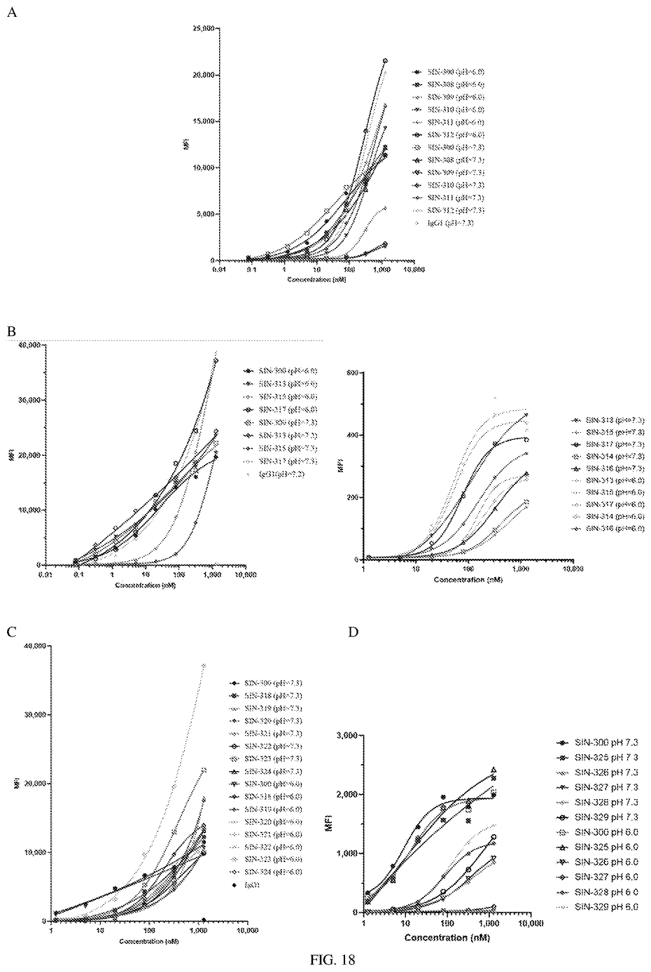

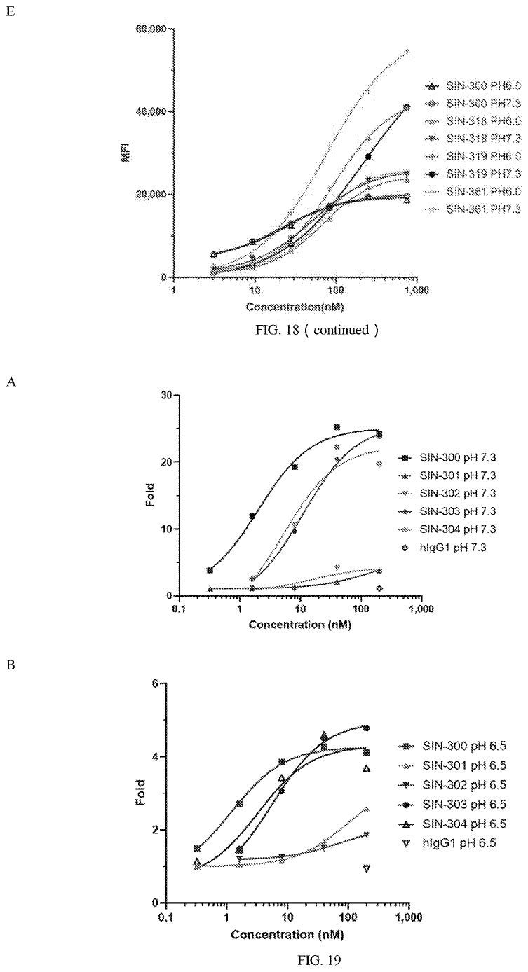

The variant SIRP IgV-IgG1 Fc fusion proteins as listed in Table 7 were selected for analysis of binding to Raji cells by FACS, similarly as detailed for SIRPαV2-IgG1 Fc fusion proteins in Example 2. The FACS results in FIG. 18A show that wild type SIRPαV1 IgV-IgG1 Fe fusion protein SIN-308 exhibited mildly weaker binding than wild type SIRPαV2 IgV-IgG1 Fc fusion protein SIN-300, but overall the mutation of K53H, R69H, Q52H or K68H similarly reduced SIRPαV1 IgV's binding to CD47 at physiological pH 7.3 but regained higher binding at an acidic pH 6.0. Meanwhile, the FACS results in FIG. 18B show that wild type SIRPαV8 IgV-IgG1 Fe fusion protein SIN-313 exhibited comparable binding to wild type SIRPαV2 IgV-IgG1 Fe fusion protein SIN-300, and overall the mutation of K53H, R69H, Q52H or K68H also reduced SIRPαV8 IgV's binding to CD47 at physiological pH 7.3 but regained higher binding at an acidic pH 6.0.

The FACS results in FIG. 18C show that wild type SIRPγ IgV-IgG1 Fe fusion protein SIN-318 exhibited much weaker binding than wild type SIRPαV2 IgV-IgG1 Fc fusion protein SIN-300, consistent with prior report (Hatherley et al., Mol Cell, 2008). Similarly, overall the mutation of K53H, R69H, Q52H or K68H reduced SIRPγ IgV's binding to CD47 at physiological pH 7.3 and regained higher binding at an acidic pH 6.0 than at pH 7.3, although to a lesser extent than the reduction of binding at pH 7.3 and regaining of binding at pH 6.0 of SIRPαV2 IgV, probably because wild type SIRPγ IgV starts with already quite low binding to CD47. Additionally mutating the 2 differing amino acids in SIRPγ IgV domain to the amino acids in corresponding positions of SIRPαV2, namely L37Q and N101D as shown in FIG. 1B, is indicated to increase SIRPγ's binding to CD47. However, only mild increase in binding was observed comparing SIRPγ IgV protein with R69H+L37Q+N101D mutation (SIN-324) to SIRPγ IgV protein with R69H only (SIN-320) (FIG. 18C). In contrast, SIRPγ IgV protein with K53H+L37Q+N101D mutation (SIN-323) exhibited largely increased binding to CD47 than SIRPγ IgV protein with K53H mutation only (SIN-319) at an acidic pH, with also large increase in binding at physiological pH but surprisingly much less than the increase at acidic pH (FIG. 18C). Notably, the SIRPγ IgV protein with K53H+L37Q+N101D mutation reached several-fold higher in binding MFI at higher concentrations than even wild type SIRPαV2 IgV-IgG1 Fc protein (SIN-300) (FIG. 18C). Similarly, the SIRPγ IgV protein with K53H+N101D mutation (SIN-361) also showed largely increased binding to CD47 than SIRPγ IgV protein with K53H mutation only (SIN-319) at an acidic pH, but more surprisingly with no significant increase in binding observed at physiological pH (FIG. 18E). On the other hand, the SIRPγ IgV protein with K53H+L37Q mutation (SIN-356) showed poor expression and low purity with only 46.51% purity in SEC-HPLC assay (versus 98.73% SEC-HPLC purity for SIN-361). This however also revealed superior expression and biophysical quality of SIRPγ IgV protein with the K53H+N101D mutation than the K53H+L37Q mutation.

The FACS results in FIG. 18D show that the SIPRβ2 IgV-IgG1 Fe fusion protein with a single mutation of H101D in the SIPRβ2 IgV domain (SIN-325) gained binding to CD47 similar to that of wild type SIRPαV2 IgV-IgG1 Fc fusion protein (SIN-300), and overall the mutation of K53H, R69H, Q52H or K68H reduced SIPRβ2 H101D IgV's binding to CD47 at physiological pH 7.3 and regained higher binding at an acidic pH 6.0. Notably, the mutations appeared to result in reduction of CD47 binding of SIPRβ2 H101D IgV at pH 7.3 to a larger extent than that of SIRPαV2 IgV, with R69H and K53H appearing to abolish SIPRβ2 H101D IgV's binding to CD47 at physiological pH 7.3. At pH 6.0, the binding of SIPRβ2 H101D IgV with K53H, R69H, Q52H or K68H also appeared to be weaker than that of SIRPαV2 IgV with the same mutation.

Altogether these results indicate that histidine substitution mutation of the conserved residue Q52, K53, K68 or R69 results in reduced CD47 binding at physiological pH but regains higher binding at an acidic pH across all subtypes of SIRPα, SIPRβ and SIRPγ.

TABLE 7
List of variant SIRPα, SIPRβ and SIPRγ IgV-IgG1 Fc fusion proteins

| | | pH sensitive mutation | SIRP IgV-IgG1 |
|---|---|---|---|
| | | on corresponding | Fc polypeptide |
| Protein name | Protein Description | template SIRP IgV | SEQ ID NO: |
| SIN-308 | SIRPαV1 WT IgV-IgG1 Fc | none | 308 |
| SIN-300 | SIRPαV2 WT IgV-IgG1 Fc | none | 300 |
| SIN-313 | SIRPαV8 WT IgV-IgG1 Fc | none | 313 |
| SIN-325 | SIRPβ2 H101D IgV-IgG1 Fc | none | 325 |
| SIN-318 | SIRPγ WT IgV-IgG1 Fc | none | 318 |
| SIN-355 | SIRPγ L37Q IgV-IgG1 Fc | none | 355 |
| SIN-360 | SIRPγ N101D IgV-IgG1 Fc | none | 360 |
| SIN-365 | SIRPγ L37Q + N101D IgV-IgG1 Fc | none | 365 |
| SIN-310 | SIRPαV1 K53H IgV-IgG1 Fc | K53H | 310 |
| SIN-301 | SIRPαV2 K53H IgV-IgG1 Fc | K53H | 301 |
| SIN-314 | SIRPαV8 K53H IgV-IgG1 Fc | K53H | 314 |
| SIN-326 | SIRPβ2 K53H + H101D IgV-IgG1 Fc | K53H | 326 |
| SIN-319 | SIRPγ K53H IgV-IgG1 Fc | K53H | 319 |
| SIN-356 | SIRPγ K53H + L37Q IgV-IgG1 Fc | K53H | 356 |
| SIN-361 | SIRPγ K53H + N101D IgV-IgG1 Fc | K53H | 361 |
| SIN-323 | SIRPγ K53H + L37Q + N101D IgV-IgG1 Fc | K53H | 323 |
| SIN-309 | SIRPαV1 R69H IgV-IgG1 Fc | R69H | 309 |
| SIN-302 | SIRPαV2 R69H IgV-IgG1 Fc | R69H | 302 |
| SIN-315 | SIRPαV8 R69H IgV-IgG1 Fc | R69H | 315 |
| SIN-327 | SIRPβ2 R69H + H101D IgV-IgG1 Fc | R69H | 327 |
| SIN-320 | SIRPγ R69H IgV-IgG1 Fc | R69H | 320 |
| SIN-357 | SIRPγ R69H + L37Q IgV-IgG1 Fc | R69H | 357 |
| SIN-362 | SIRPγ R69H + N101D IgV-IgG1 Fc | R69H | 362 |
| SIN-324 | SIRPγ R69H + L37Q + N101D IgV-IgG1 Fc | R69H | 324 |
| SIN-312 | SIRPαV1 Q52H IgV-IgG1 Fc | Q52H | 312 |
| SIN-303 | SIRPαV2 Q52H IgV-IgG1 Fc | Q52H | 303 |
| SIN-316 | SIRPαV8 Q52H IgV-IgG1 Fc | Q52H | 316 |
| SIN-329 | SIRPβ2 Q52H + H101D IgV-IgG1 Fc | Q52H | 329 |
| SIN-322 | SIRPγ Q52H IgV-IgG1 Fc | Q52H | 322 |
| SIN-358 | SIRPγ Q52H + L37Q IgV-IgG1 Fc | Q52H | 358 |
| SIN-363 | SIRPγ Q52H + N101D IgV-IgG1 Fc | Q52H | 363 |
| SIN-366 | SIRPγ Q52H + L37Q + N101D IgV-IgG1 Fc | Q52H | 366 |
| SIN-311 | SIRPαV1 K68H IgV-IgG1 Fc | K68H | 311 |
| SIN-304 | SIRPαV2 K68H IgV-IgG1 Fc | K68H | 304 |
| SIN-317 | SIRPαV8 K68H IgV-IgG1 Fc | K68H | 317 |
| SIN-328 | SIRPβ2 K68H + H101D IgV-IgG1 Fc | K68H | 328 |
| SIN-321 | SIRPγ K68H IgV-IgG1 Fc | K68H | 321 |
| SIN-359 | SIRPγ K68H + L37Q IgV-IgG1 Fc | K68H | 359 |
| SIN-364 | SIRPγ K68H + N101D IgV-IgG1 Fc | K68H | 364 |
| SIN-367 | SIRPγ K68H + L37Q + N101D IgV-IgG1 Fc | K68H | 367 |

### Example 7. Antibody Dependent Cell-Mediated Phagocytosis (ADCP) Activities of Variant SIRPαV2

IgV-IgG1 Fc fusion proteins measured by ADCP Jurkat reporter assay 5 To functionally test the pH-dependent binding of SIRPαV2 IgV variant proteins, the wild type and variant SIRPαV2 IgV-IgG1 Fc fusion proteins were subjected to an ADCP Jurkat reporter assay using a Jurkat-NFAT-Luc2-CD32a-R167 ADCP reporter cell line (KYinno, CAT #KC-1524), which expresses CD32a-R167 receptor to transmit ADCP activation signal and activate NFAT-Luc2 luciferase reporter gene, giving rise to chemiluminescence after substrate addition. The ADCP activity of the test articles can be determined by measuring the signal of luminescence using a luciferase assay kit. Raji cells were used as target cells and plated at 20,000/well in 96-well plate in RPMI 1640 medium at pH 7.3 or 6.5. Five-fold serially diluted concentrations of wild type and variant SIRPαV2 IgV-IgG1 Fe fusion proteins at pH 7.3 or 6.5 were added to Raji cells and incubated at 37° C. for 30 minutes. The Jurkat-NFAT-Luc2-CD32a-R167 ADCP reporter cell at pH 7.3 or 6.5 were then added to the mixture at 100,000/well and incubated at 37° C. for 6 hours. Bright-Glo reagent was then added to the cells and luminescence signal was read out using a multi-mode microplate reader. ADCP activity was calculated as the fold of luminescence strength of a test article over that of buffer negative control (Raji cells+Jurkat report cells+buffer control without test article).

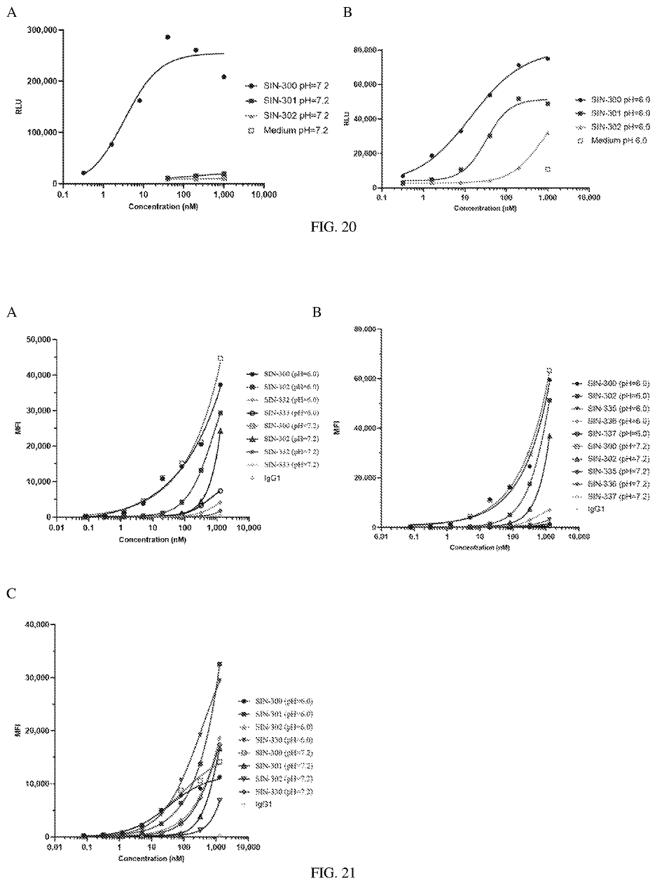

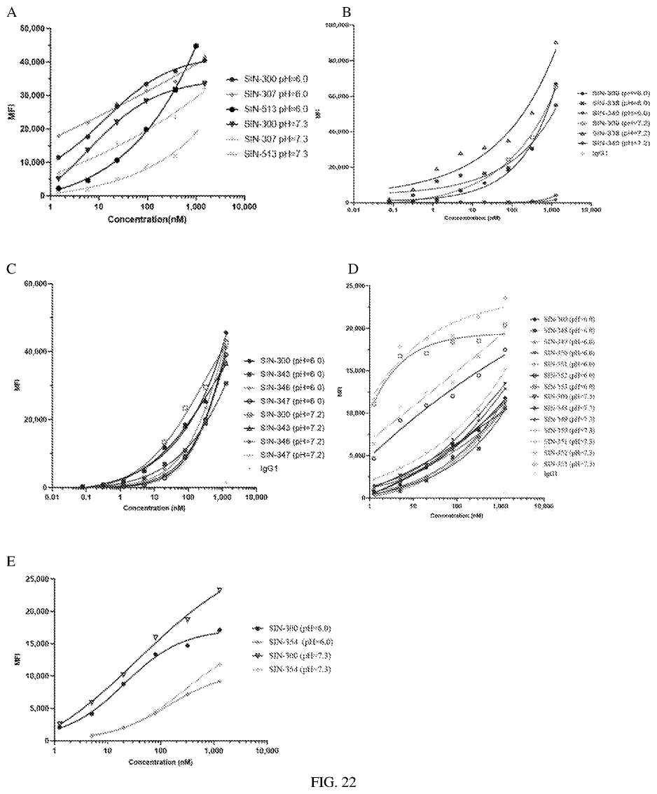

The results in FIG. 19A show that at physiological pH 7.3, wild type protein SIN-300 exhibited robust ADCP activity, but SIN-301 (K53H) and SIN-302 (R69H) exhibited minimal ADCP activity less than ⅙ of that of SIN-300 at high concentration, while SIN-302 (Q52H) and SIN-304 (K68H) exhibited significant ADCP activity but weaker than that of SIN-300. At an acidic pH 6.5, the results in FIG. 19B show that ADCP activity readout of the assay was largely reduced overall at pH 6.5, likely due to suppressed activity of the Jurkat reporter cells at an acidic pH, so that the peak fold of wild type SIN-300 at pH 6.5 is only ~1/6 of that at pH 7.3. However, the activity fold of SIN-301 at pH 6.5 reached ~60% of the peak level of wild type SIN-300 at high concentration, and the activity fold of SIN-302 at pH 6.5 also reached ~40% of the peak level of wild type SIN-300 at high concentration. Similarly, SIN-303 and SIN-304 showed activity closer to that of wild type SIN-300 at pH 6.5 than at pH 7.3. These functional ADCP results are consistent with the reduced binding of the variant SIRPαV2 IgV proteins to CD47 at physiological pH but higher binding at an acidic pH.

### Example 8. Antibody Dependent Cell-Mediated Cytotoxicity (ADCC) Activities of Variant SIRPαV2 IgV-IgG1 Fc Fusion Proteins Measured by ADCC Jurkat Reporter Assay

To further functionally test the pH-dependent binding of SIRPαV2 IgV variant proteins, the wild type (SIN-300) and variant SIRPαV2 IgV-IgG1 Fc fusion proteins (SIN-301 and SIN-302) were subjected to an ADCC Jurkat reporter assay using a Jurkat-NFAT-Luc2-CD16 V158 ADCC reporter cell line, which expresses CD16-V158 receptor to transmit ADCC activation signal and activate NFAT-Luc2 luciferase reporter gene, giving rise to chemiluminescence after substrate addition. The ADCC activity of the test articles can be determined by measuring the signal of luminescence using a luciferase assay kit. Raji cells were used as target cells and plated at 20,000/well in 96-well plate in RPMI 1640 medium at pH 7.3 or 6.5. Five-fold serially diluted concentrations of wild type and variant SIRPαV2 IgV-IgG1 Fe fusion proteins at pH 7.2 or 6.0 were added to Raji cells and incubated at 37° C. for 30 minutes. The Jurkat-NFAT-Luc2-CD16 V158 ADCC reporter cell at pH 7.2 or 6.0 were then added to the mixture at 100,000/well and incubated at 37° C. for 6 hours. Bright-Glo reagent was then added to the cells and luminescence signal was read out using a multi-mode microplate reader.

The results in FIG. 20A show that at physiological pH 7.2, wild type protein SIN-300 exhibited robust ADCC activity, but SIN-301 (K53H) and SIN-302 (R69H) exhibited negligible ADCC activity. At an acidic pH 6.0, the results in FIG. 20B show that ADCC activity readout of the assay was largely reduced overall, likely due to suppressed activity of the Jurkat reporter cells at an acidic pH, so that the peak activity of wild type SIN-300 at pH 6.0 was only ~1/3 of that at pH 7.2. However, the ADCC activity of SIN-301 at pH 6.0 reached ~65% of the peak level of wild type SIN-300 at high concentration, and the activity of SIN-302 at pH 6.0 also reached ~40% of the peak level of wild type SIN-300 at high concentration. These functional ADCC results are consistent with the ADCP results in Example 7 and further functionally validate the reduced binding of the variant SIRPαV2 IgV proteins to CD47 at physiological pH but higher binding at an acidic pH.

### Example 9. Generation and Binding Activities of Variant SIRPαV2 IgV with Combo Mutations Measured by FACS

Combination of single mutations are then tested to identify positive combo mutations that further reduce the binding at physiological pH, and/or further increase the binding of the variant protein at an acidic pH. SIRPαV2 IgV-IgG1 Fc fusion proteins with various combo mutations comprising two or more mutations selected from K53H, R69H, Q52H and K68H on the wild type SIRPαV2 IgV template (SEQ ID NO: 4) were designed. Initially, exemplary proteins with double mutations of SIRPαV2 IgV domain, as listed in Table 8 below, were produced similarly as done in Example 1 and analyzed for binding to CD47 expressing Raji cells by FACS similarly as done in Example 2. The same double mutations of other SIRPα, SIRPβ or SIRPγ IgV domains can also be produced and analyzed for CD47 binding similarly.

The results in FIG. 21A-C show that all the combo mutations exhibited further reduced binding at physiological pH 7.2 than the corresponding single mutation component, which is desirable to further reduce or minimize binding to the ubiquitous CD47-expressing normal tissues and cells. Meanwhile at an acidic pH 6.0, except the variant SIN-337 (K53H+R69H) that showed no binding, all the combo mutation variants SIN-330 (Q52H+K68H), SIN-332 (K53H+Q52H), SIN-333 (K53H+K68H), SIN-335 (R69H+Q52H) and SIN-336 (R69H+K68H), continued to exhibit higher binding than at pH 7.2. Surprisingly, the higher binding at an acidic pH of the two single mutations didn't cooperate to increase binding of the double mutation at an acidic pH 6.0 than the two single mutations, but instead decreased the binding at an acidic pH 6.0.

TABLE 8
List of variant SIRPαV2 IgV-IgG1 Fc fusion proteins with double mutations produced

| | | pH sensitive | SIRPαV2 IgV- |
|---|---|---|---|
| | | mutation on wild | IgG1 Fc |
| | | type SIRPαV2 IgV | polypeptide |
| Protein name | Protein description | (SEQ ID NO: 4) | SEQ ID NO: |
| SIN-330 | SIRPαV2 Q52H + K68H IgV-IgG1 Fc | Q52H + K68H | 330 |
| SIN-332 | SIRPαV2 K53H + Q52H IgV-IgG1 Fc | K53H + Q52H | 332 |
| SIN-333 | SIRPαV2 K53H + K68H IgV-IgG1 Fc | K53H + K68H | 333 |
| SIN-335 | SIRPαV2 R69H + Q52H IgV-IgG1 Fc | R69H + Q52H | 335 |
| SIN-336 | SIRPαV2 R69H + K68H IgV-IgG1 Fc | R69H + K68H | 336 |
| SIN-337 | SIRPαV2 R69H + K53H IgV-IgG1 Fc | R69H + K53H | 337 |

Next, combination of mutations at additional residues of SIRP IgV domain on top of a backbone mutation of K53H, R69H, Q52H, K68H, Q52H+K68H, K53H+K68H, R69H+K68H, K53H+Q52H, R69H+Q52H, K53H+K68H+Q52H, or R69H+K68H+Q52H, as listed in Table 9 below, are designed to further increase CD47 binding at an acidic pH, and/or further reduce CD47 binding at physiological pH. In one category, candidate residues located at the interaction interface between SIRP IgV domain and CD47 are selected, as they may directly affect SIRP IgV domain's binding to CD47 and/or the backbone mutations' interaction with CD47, based on the crystal structure of SIRPαV2 IgV-CD47 complex (PDB: 2JJS). In another category, additional candidate residues located outside the direct interaction interface are selected for their potential to indirectly affect SIRP IgV domain's binding to CD47 through affecting the SIRP IgV domain's confirmation/structure and/or the backbone mutations' interaction with CD47, based on the crystal structure of SIRPαV2 IgV-CD47 complex (PDB: 2JJS). Exemplary SIRPαV2 IgV-IgG1 Fe fusion proteins (configured as in format FV-2) with single mutation at selected additional residue I31, P35, R40, R46, R59, S66, K96, S98, P99 or K104 of the wild type SIRPαV2 IgV (SEQ ID NO: 4), as shown in Table 9 below, were produced similarly as done in Example 1, and analyzed for binding to CD47-expressing Raji cells by FACS assay similarly as done in Example 2. The same mutations at the corresponding positions of other SIRPα, SIRPβ or SIRPγ IgV domains can also be produced and analyzed for CD47 binding similarly, with one additional residue in position numbering after residue 100 in SIRPβ, SIRPγ and some SIRPαIgV (e.g. SEQ ID NO: 1-3, 6-8, and 11-21) than in SIRPαV2 IgV (SEQ ID NO: 4). For example, as shown in FIGS. 1A and 1B, K104 in SIRPαV2 IgV is K105 correspondingly in SIRPαV1, SIRPαV4, SIRPαV5, SIRPαV6, SIRPαV9, SIRPβ and SIRPγ.

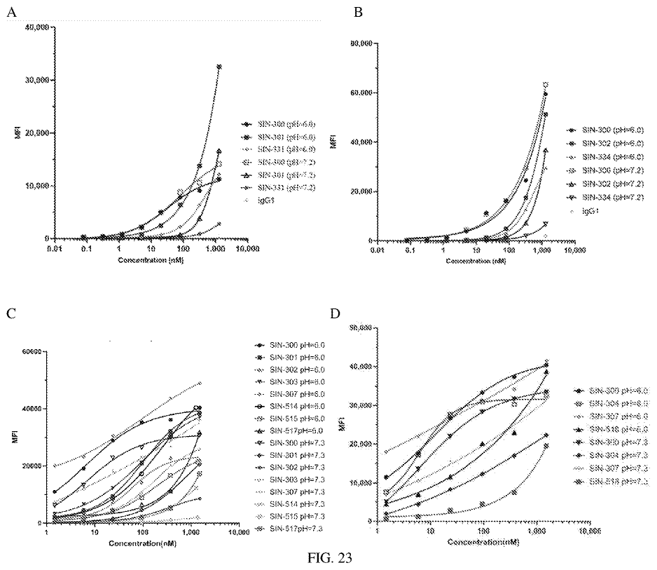

As shown in the results in Table 10, these proteins were well expressed. The results in FIG. 22A-E, together with the results in FIG. 14A-D, show that the additional single mutations of the parent SIRPαV2 IgV (SEQ ID NO: 4) tested exhibit several types of effects on the SIRP IgV domain's binding to CD47, as summarized as subgroups in Table 9:1) Subgroup 1 mutations such as I31E in SIN-307 and I31D in SIN-S13, similar to the mutation of K53H, R69H, Q52H and K68H, exhibited higher binding to CD47 at an acidic pH than at physiological pH (FIG. 22A). Notably, I31E reduced CD47 binding of SIRP IgV at physiological pH without notable impact at an acidic pH (FIG. 22A), a preferred property also applicable to help further lower CD47-binding at physiological pH when combined with the backbone mutation (K53H, R69H, Q52H, K68H or Q52H+K68H). Meanwhile, I31D also largely reduced CD47 binding at physiological pH but with less reduction at an acidic pH (FIG. 22A). Similar to the effect in SIRPα IgV, the corresponding mutation of I31E or I31D in SIRPβ IgV and the corresponding mutation of L31E or L31D in SIRPγ IgV can similarly lead to higher binding to CD47 at an acidic pH than at physiological pH. Furthermore, the result in FIG. 23C showed that the SIRPαV2 IgV protein (SIN-514) comprising both K53H and I31E mutation exhibited further lowered binding to CD47 at physiological pH but with similar binding at an acidic pH than SIRPαV2 IgV protein (SIN-301) comprising K53H only. Additionally, the results in FIG. 23C-D also showed that with addition of the I31E mutation, SIRPαV2 IgV protein comprising both R69H+I31E mutation (SIN-515), or Q52H+I31E mutation (SIN-517), or K68H+I31E mutation (SIN-518) also exhibited further lowed binding to CD47 at physiological pH than SIRPαV2 IgV protein comprising R69H only (SIN-302), Q52H only (SIN-303) or K68H only (SIN-304) respectively, while maintaining higher binding to CD47 at an acidic pH than at physiological pH. Similarly, addition of the I31D mutation to the mutation of K53H, R69H, Q52H or K68H can further lower binding of the SIRP IgV to CD47 at physiological pH, while maintaining higher binding to CD47 at an acidic pH than at physiological pH. Also similarly, addition of the mutation of L31E or L31D to the mutation of K53H, K53H+N101D or K53H+N101D+L37Q in SIRPγ IgV can further lower the binding to CD47 at physiological pH. 2) Subgroup 2 mutations, such as in SIN-348 (R40H), SIN-342 (N51H), SIN-343 (S66H), SIN-354 (K96R), SIN-345 (G97H), SIN-346 (S98H), SIN-347 (P99H) and SIN-351 (K104H), reduced CD47 binding at both physiological pH and acidic pH (FIG. 14C-D and FIG. 22β-E), a property applicable to help further lower CD47-binding at physiological pH when combined with the backbone mutation (K53H, R69H, Q52H, K68H or Q52H+K68H); 3) Subgroup 3 mutations, such as in SIN-339 (V33H), SIN-340 (P35H) and SIN-305 (K96H), reduce substantially or abolish CD47 binding at both physiological pH and acidic pH (FIG. 22B and FIG. 14A-C); 4) Subgroup 4 mutations, such as in SIN-341 (Q37H), SIN-349 (R46H), SIN-344 (E70H) and SIN-306 (M72H), didn't notably affect CD47 binding at both physiological pH and acidic pH (FIG. 22β-D and FIG. 14A-D). However, when Q37H was combined with the backbone mutation (K53H or R69H), surprisingly, it further lowered CD47 binding at physiological and acidic pH (FIG. 23A-B), indicating that single mutation of SIRP IgV that doesn't impact CD47 binding on its own could still affect CD47 binding when combined with the backbone mutation (K53H, R69H, Q52H, K68H or Q52H+K68H); 5) Subgroup 5 mutations, such as in SIN-353 (I31W), SIN-352 (I31Y) and SIN-350 (R59H), increased CD47 binding at both physiological pH and acidic pH (FIG. 22E). In addition, the deglycosylation mutation at residue N80 (N80A, N80G, N80S, N80Q) is reported to remove glycosylation of SIRP IgV and increase CD47 binding (see U.S. Pat. No. 1,080,082B2). Thus, this subgroup of mutations is applicable to help further increase CD47-binding at an acidic pH when combined with the backbone mutation (K53H, R69H, Q52H, K68H, Q52H+K68H, K53H+K68H, R69H+K68H, K53H+Q52H, R69H+Q52H, K53H+K68H+Q52H, or R69H+K68H+Q52H). 6) Subgroup 6 mutations, such as in SIN-338 (I31H), increased CD47 binding at physiological pH (FIG. 22B); Additionally, the single mutations in Subgroup 7 and 9 of Table 9 were reported to reduce or increase CD47 binding respectively (see U.S. Pat. No. 11,021,694B2 and U.S. Pat. No. 10,800,821B2), a property applicable to help further decrease CD47-binding at physiological pH or increase CD47-binding respectively at an acidic pH when combined with the backbone mutation (K53H, R69H, Q52H, K68H, Q52H+K68H, K53H+K68H, R69H+K68H, K53H+Q52H, R69H+Q52H, K53H+K68H+Q52H, or R69H+K68H+Q52H). Together, these results showcase mutations at a large range of additional residues of SIRP IgV domain for developing combo mutations with the backbone mutation (K53H, R69H, Q52H, K68H, R69H+Q52H, Q52H+K68H, K53H+K68H, R69H+K68H, K53H+Q52H, K53H+K68H+Q52H, or R69H+K68H+Q52H) to further modulate CD47 binding at physiological and/or acidic pH, as shown in Table 9. Certain exemplary combo mutations are also provided in SEQ ID NO: 22 to 113. Additionally, the backbone mutation (K53H, R69H, Q52H, K68H, Q52H+K68H, K53H+K68H, R69H+K68H, K53H+Q52H, R69H+Q52H, K53H+K68H+Q52H, or R69H+K68H+Q52H) or the combo mutations as shown in Table 9 herein are amenable for combination with additional mutations of SIRP IgV domains known in the arts. For example, a backbone mutation (K53H, R69H, Q52H, K68H, Q52H+K68H, K53H+K68H, R69H+K68H, K53H+Q52H, R69H+Q52H, K53H+K68H+Q52H, or R69H+K68H+Q52H) or a combo mutations as shown in Table 9 herein is applicable to be added to any SIRP IgV-comprising proteins such as ALX148, 1D4, 1A5, 2D3, 2A10, 2B5, 2A2, 2F5, FB3, FD6, FA4, CV1, AS1, AS2, SG3847, CD001, CD002, 106, IMM02, IMM0306, IMM2902, IMM2502, IMM4701C, IMM5601, IMM6101, IMM2505, HX009, DSP-107, JMT601, SL-172154, SG-404, SG-2501 or SG12473 (as set forth in Table 11) to generate de novo or modulate existing pH-sensitive CD47-binding of these proteins.

TABLE 9
Combination of additional mutation(s) with a backbone mutation

| | Combo of one or more | | Effect of the additional |
|---|---|---|---|
| Backbone | additional mutation(s) below | Subgroups of the additional | single mutation versus |
| mutation | with the backbone mutation | mutations | wild type parent domain |
| One of the | E3H/G3H, L4H, L4V, L4I, | Subgroup 1: I31E/L31E, I31D/L31D | Reduce CD47 binding at physiological |
| K53H, R69H, | V6H/M6H, V6I/M6I, V6L/M6L, | | pH |
| Q52H, K68H, | D10H/E10H, K11H, V15H, | Subgroup 2: R40H, N51H, S66H/L66H, | Reduce CD47 binding at both |
| Q52H + K68H, | E19H/K19H, A21H, A21V, | K96R, G97H, S98H, P99H, K104H/K105H | physiological pH and acidic pH |
| K53H + K68H, | V27H/A27H, V27I/A27I, | Subgroup 3: V33H, P35H, K96H | Reduce CD47 binding substantially at |
| R69H + K68H, | V27L/A27L, V27Q/A27Q, S29H, | | both physiological pH and acidic pH |
| K53H + Q52H, | L30H, I31E/L31E, I31D/L31D, | Subgroup 4: Q37H/L37H, R46H, | No notable change in CD47 binding at |
| R69H + Q52H, | I31Q/L31Q, I31N/L31N, | E70H/N70H, M72H/L72H | both physiological pH and acidic pH |
| K53H + K68H | I31H/L31H, I31Y/L31Y, | Subgroup 5: I31Y/L31Y, I31W/L31W, | Increase CD47 binding at both |
| HQ52H, or | I31W/L31W, I31F/L31F, | R59H, N80A, N80G, N80S, N80Q | physiological pH and acidic pH |
| R69H + | I31V/L31V, I31R/L31R, | Subgroup 6: I31H/L31H | Increase CD47 binding at |
| K68H + | I31K/L31K, I31T/L31T, | | physiological pH |
| Q52H | I31S/L31S, I31L, P32H, V33H, | Subgroup 7: V27Q/A27Q, P35G, P35N, | Single mutation reported to reduce |
| mutation | V33I, G34H, P35H, P35G, | Q37A/L37A, Q37V/L37V, E47Y, Q52E, | CD47 binding (see. U.S. Pat. No. |
| | P35N, 136H/V36H, Q37H/L37H, | H56Y, S66E/L66E, S66W/L66W, T67E, | 11,021,694B2) |
| | Q37V/L37V, Q37W/L37W, | T67W, K68A, K68E, K68I, K68T, | |
| | Q37A/L37A, W38H, F39H, R40H, | M72I/L72I, M72N/L72N, M72W/L72W, | |
| | R46H, E47H, E47V, E47L, E47Y, | V92N | |
| | E47Q, L48H, I49H, Y50H, N51H, | Subgroup 8: E54P, M72R/L72R | Single mutation reported to not |
| | Q52E, Q52G, K53R, E54H, E54D, | | notably affect CD47 binding (see. |
| | E54Q, E54N, E54P, E54S, E54G, | | U.S. Pat. No. 11,021,694B2) |
| | H56P, H56Y, H56R, F57H, R59H, | Subgroup 9: V6I/M6I, V27I/A27I, | Single mutation reported to increase |
| | V60H, T61H, T62H, V63I, V63A, | I31R/L31R, I31T/L31T, Q37W/L37W, | CD47 binding (see. |
| | S64H, E65H/D65H, E65R/D65R, | H56P, S66Q/L66Q, N80A | U.S. Pat. No. 11,021,694B2; |
| | S66H/L66H, S66T/L66T, | | U.S. Pat. No. 10,800,821B2) |
| | S66G/L66G, S66E/L66E, | Subgroup 10: L4V, L4I, V6I, V6L, A21V, | Reported to increase CD47 binding in |
| | S66Q/L66Q, S66W/L66W, | V27I/A27I, V27L/A27L, I31F, I31T, I31S, | combo mutations (see Science. 2013 |
| | S66A/L66A, T67H, T67E, T67W, | I31L, V33I, E47V, E47L, E47Q, Q52G, | Jul. 5; 341(6141): 88-91; |
| | K68R, K68A, K68E, K68I, K68T, | K53R, E54Q, E548, E54N, E54D, E54G, | US20210171593A1; |
| | E70H/N70H, E70D/N70D, N71H, | H56P, H56R, V63I, V63A, D65H, D65R, | CN111253482B; CN109306017B) |
| | M72H/L72H, M72N/L72N, | S66T/L66T, S66G/L66G, S66A/L66A, | |
| | M72R/L72R, M72I/L72I, | K68R, N70D, R77N, R77K, G79H, V92I, | |
| | M72W/L72W, F74H, S75H/P75H, | V92S, F94L, F94V, F103V, F103I | |
| | I76H, S77H/R77H, S77K/R77K, | Subgroup 11: S29H, L30H, P32H, | Additional candidate mutations located |
| | S77N/R77N, S79H/G79H, N80A, | G34H, E54H, T61H, T62H, T67H, F74H, | within SIRP IgV domain and CD47 |
| | N80G, N80S, N80Q, N80H/S80H, | S77H/R77H, S79H, K93H | interation interface region that may |
| | D85H, Y89H, V92H, V92I, V92N, | | directly affect SIRP IgV domain's |
| | V928, K93H, F94H, F94L, F94V, | | binding to CD47 and/or the backbone |
| | R95H, K96H, K96R, G97H, S98H, | | mutations' interaction with CD47 |
| | P99H, E102H/E103H, | Subgroup 12: E3H/G3H, L4H, V6H/M6H, | Additional candidate mutations that |
| | F103H/F104H, F103V/F104V, | D10H/E10H, K11H, V15H, E19H/K19H, | may indirectly affect SIRP IgV |
| | F103I/F104I, K104H/K105H, | A21H, V27H/A27H, I36H/V36H, W38H, | domain's binding to CD47 through |
| | L111H/L112H/M112H, | F39H, E47H, L48H, 149H, Y50H, F57H, | affecting the SIRP IgV domain's |
| | V113H/V114H/L114H, | V60H, S64H, E65H, N71H, S75H/P75H, | structure/confirmation and/or the |
| | R114H/R115H/G115H, | I76H, N80H/S80H, D85H, Y89H, V92H, | backbone mutations' interaction with |
| | K116H/K117H | F94H, R95H, E102H/E103H, F103H/F104H, | CD47 |
| | | L111H/L112H/M112H, | |
| | | V113H/V114H/L114H, | |
| | | R114H/R115H/G115H, K116H/K117H | |

Note:
The “/” indicates different mutation changes at the corresponding amino acid position across SIRPα, SIRPβ and SIRPγ IgV domains, due to different parent amino acid residues and/or position numbering for the mutation at the corresponding position, according to the SIRPα, SIRPβ and SIRPγ IgV amino acid sequences provided in SEQ ID NO: 1 to 21.

TABLE 10
Transient expression of additional single-mutation variant SIRPαV2 IgV-IgG1 Fc fusion protein

| | SIN- | SIN- | SIN- | SIN- | SIN- | SIN- | SIN- | SIN- | SIN- | SIN- | SIN- | SIN- | SIN- | SIN- |
|---|---|---|---|---|---|---|---|---|---|---|---|---|---|---|
| Protein name | 307 | 513 | 338 | 352 | 353 | 340 | 348 | 349 | 350 | 343 | 354 | 346 | 347 | 351 |
| Mutation on | 131E | I31D | 131H | 131Y | 131W | P35H | R40H | R46H | R59H | S66H | K96R | S98H | P99H | K104H |
| template | | | | | | | | | | | | | | |
| SIRPαV2 | | | | | | | | | | | | | | |
| IgV (SEQ ID | | | | | | | | | | | | | | |
| NO: 4) | | | | | | | | | | | | | | |
| SIRPαV2 | 307 | 513 | 338 | 352 | 353 | 340 | 348 | 349 | 350 | 343 | 354 | 346 | 347 | 351 |
| IgV-IgG1 Fc | | | | | | | | | | | | | | |
| SEQ ID NO: | | | | | | | | | | | | | | |
| Yield (mg/L) | 127.3 | 130 | 12.7 | 126 | 147.7 | 116.7 | 126.7 | 147.3 | 127.3 | 92.3 | 147.3 | 198.7 | 140 | 91.3 |
| Molecular | 80 KD | 80 KD | 80 KD | 80 KD | 80 KD | 80 KD | 80 KD | 80 KD | 80 KD | 80 KD | 80 KD | 80 KD | 80 KD | 80 KD |
| weight (by | | | | | | | | | | | | | | |
| SDS-PAGE) | | | | | | | | | | | | | | |
| SDS-PAGE | >95% | >95% | >95% | >95% | >95% | >95% | >95% | >95% | >95% | >95% | >95% | >95% | >95% | >95% |
| purity | | | | | | | | | | | | | | |
| SEC-HPLC | 99.2% | 100% | 99.3% | 99.2% | 99.2% | 98.9% | 99.2% | 98.6% | 98.5% | 99.2% | 99.5% | 99.0% | 98.7% | 99.2% |
| purity | | | | | | | | | | | | | | |

TABLE 11
List of exemplary proteins known in the arts that comprise a SIRPα IgV domain

| Protein Name | Reference patent | Reference article | Developer |
|---|---|---|---|
| TTI-621 | WO2014094122A1 | J Hematol Oncol. 2021 Oct. 30; 14(1): 180 | Trillium Therapeutics, Inc. |
| TTI-622 | WO2014094122A1 | J Hematol Oncol. 2021 Oct. 30; 14(1): 180 | Trillium Therapeutics, Inc. |
| IMM01 | U.S. Pat. No. | J Hematol Oncol. 2021 Oct. 30; 14(1): 180 | Immuneonco |
| | 10,800,821B2 | | Biopharmaceutical |
| SG3847 | US20210171593A1 | | Hangzhou Sumgen Biotech |
| 106 | CN111253482B | | Academy of Military Medical |
| | | | Sciences AMMS of PLA |
| CD001, CD002 | CN1093060178 | | Beierda Pharmacy Suzhou Co. |
| ALX148 | US20170107270A1; | J Hematol Oncol. 2021 Oct. 30; 14(1): 180 | Stanford University, ALX |
| | WO2013109752A1 | | Oncology, Inc. |
| 1D4, 1A5, 2D3, 2A10, | WO2013109752A1 | Science. 2013 Jul. 5; 341(6141): 88-91 | |
| 2B5, 2A2, 2F5, FB3, | | | |
| FD6, FA4, CV1 | | | |
| AS1, AS2 | WO2016024021A1 | | |
| IMM02, IMM0306, | U.S. Pat. No. | J Hematol Oncol. 2021 Oct. 30; 14(1): 180; | Immuneonco |
| IMM2902, IMM2502, | 10,800,821B2 | Front Immunol. 2020 Jan. 28; 11: 18 | Biopharmaceuticals |
| IMM4701C, IMM5601, | | | (Shanghai) Co. |
| IMM6101 | | | |
| IMM2505 | WO2019080883A1 | | Immuneonco |
| | | | Biopharmaceuticals; Shenghe |
| | | | Biologicals |
| HX009 | CN109897111A | J Hematol Oncol. 2021 Oct. 30; 14(1): 180 | Hanx Biopharmaceutical Co. |
| JMT601 | WO2018205936A1 | J Hematol Oncol. 2021 Oct. 30; 14(1): 180 | Shanghai JMT-Bio Technology |
| DSP-107 | WO2018127919A1 | J Hematol Oncol. 2021 Oct. 30; 14(1): 180 | KAHR Medical |
| SL-172154 | US20200071380A1 | | Shattuck Labs |
| SG-404, SG-2501, | US20210171593A1 | | Hangzhou Sumgen Biotech |
| SG12473 | | | |

### Example 10. PH-Differential CD47-Binding of Multivalent Fusion Proteins Comprising Variant SIRP IgV Domains

In addition to the Fc-fusion proteins comprising two separated SIRP IgV monomers shown in examples above, SIRP IgV multimer polypeptide comprising two, three, four or more serially linked SIRP IgV monomers are configured in exemplary structural format variants as illustrated in FIG. 3 (FV-19a to c) and constructed in exemplary compositions as depicted in Table 17 and 18. Also, monospecific Fe fusion proteins comprising two, four, six or more SIRP IgV monomers are configured in exemplary structural format variants as illustrated in FIG. 2 and constructed in exemplary compositions as depicted in Table 12 and 19. Additionally, multispecific fusion proteins comprising one, two, three, four or more SIRP IgV monomers and at least one non-CD47 binding domain are configured in exemplary structural format variants as illustrated in FIGS. 3 to 12 and constructed in exemplary compositions as depicted in Table 13 to 17. Full amino acid sequence compositions of exemplary proteins are further described in Table 18 to 21. While the exemplary proteins in Table 18 to 21 use SIRPαV2 IgV specifically, it's straightforward to replace the SIRPαV2 IgV domain with other SIRPα (SIRPα1, SIRPα3, SIRPα4, SIRPα5, SIRPα6, SIRPα7, SIRPα8, SIRPα9 or SIRPα 10), SIRPβ or SIRPγ IgV domains with the same substitution at the corresponding residue positions (sequence alignment shown in FIG. 1A-B), and produce all the proteins similarly as described in the General Methods and Examples above. Together, these proteins represent a range of valency of SIRP IgV monomers in different formats to analyze the effect of different valency of SIRP IgV monomers comprising different mutations on differential CD47-binding at an acidic pH and physiological pH through FACS binding assay and/or ELISA assay as described in the General Methods and Examples above, by comparing the binding at an acidic pH and physiological pH of proteins: 1) having the same valency of SIPR IgV monomers but different mutations (i.e. different variant SIRP IgV monomers) or no such mutation (i.e. parent SIRP IgV monomers) to identify mutations that exhibit higher binding to CD47 at an acidic pH than at physiological pH at various valency; and 2) having the same mutations of SIRP IgV monomers but different valencies to identify a valency wherein the protein exhibits increased binding at an acidic pH and also maintains similar or larger binding difference at an acidic pH than at physiological pH, comparing to that at bivalency. While a higher valency may enable stronger binding, it is unknown how the pH-differential binding of a variant SIRP IgV domain exhibited between at an acidic pH and physiological pH at a given valency may behave at a higher or lower valency. To test, the proteins constructed with a range of valency of SIRP IgV monomers in different formats are used. The expression and production of these proteins in HEK293 cells are performed similarly as described in the General Methods and in Example 1 and 9, except that for proteins without Fe, a C-terminal 6×-His tag is added to purify the protein through 1-step Ni-NTA purification.

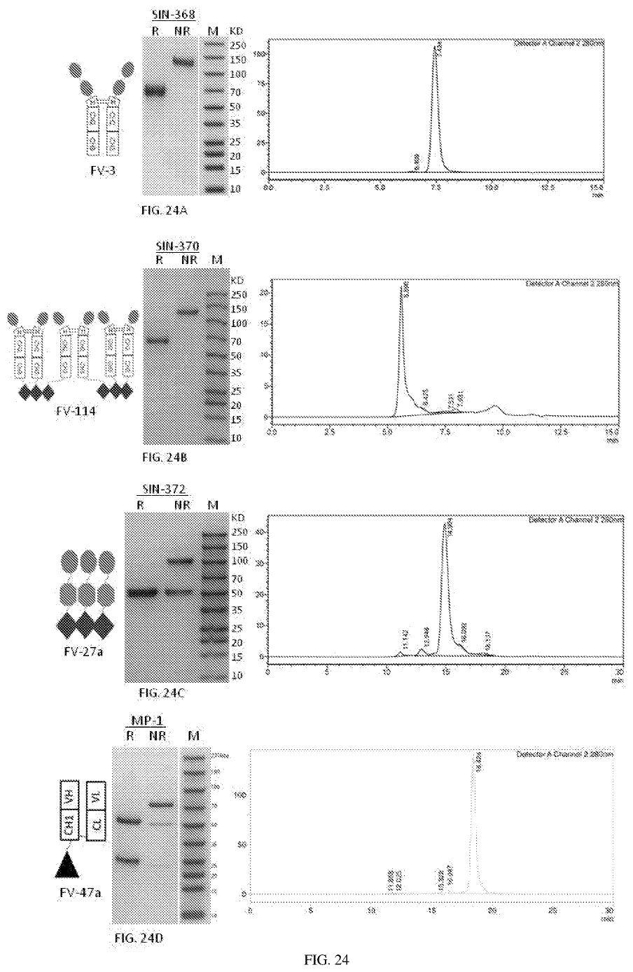

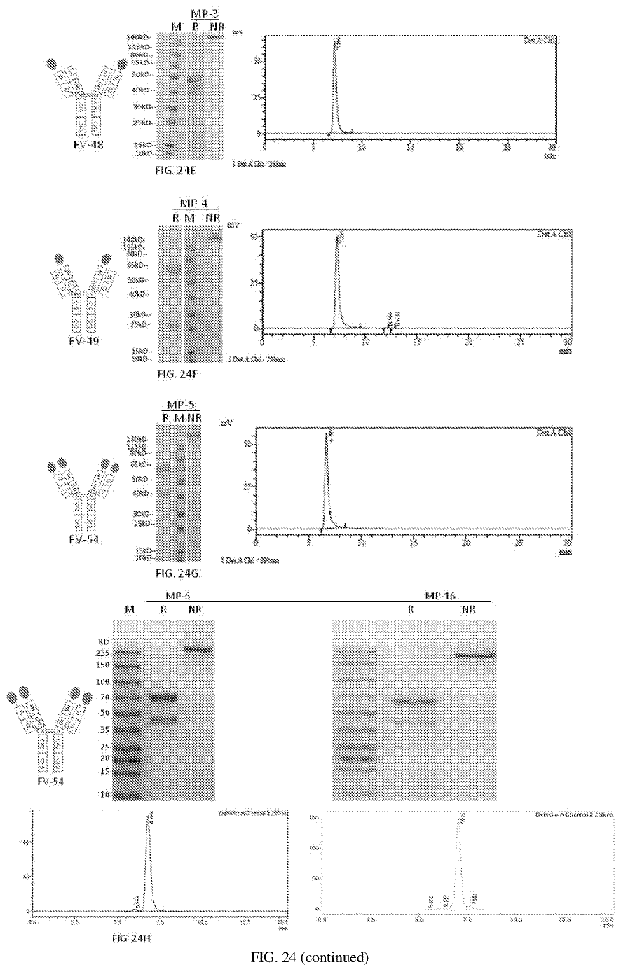

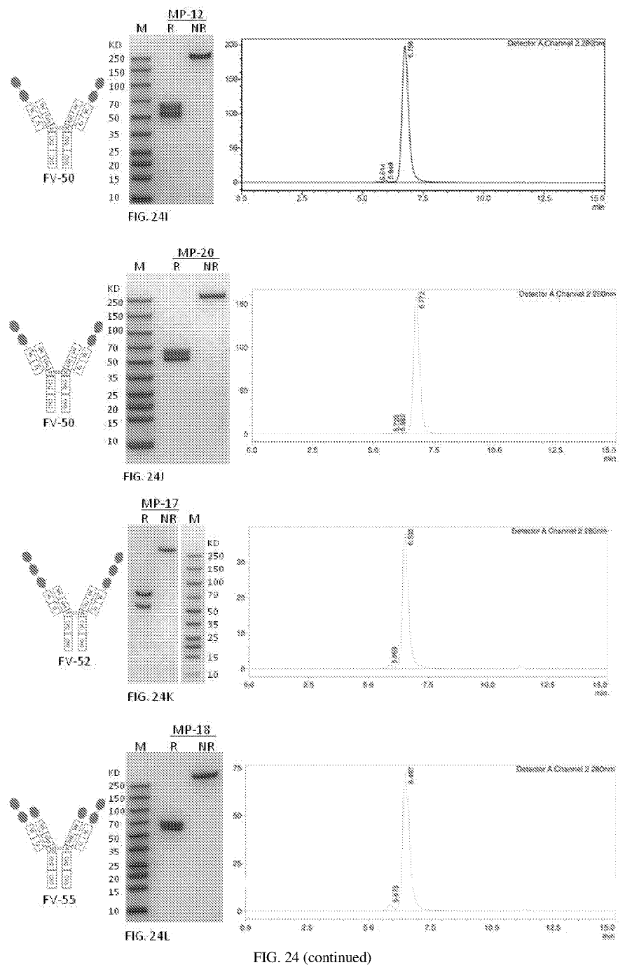

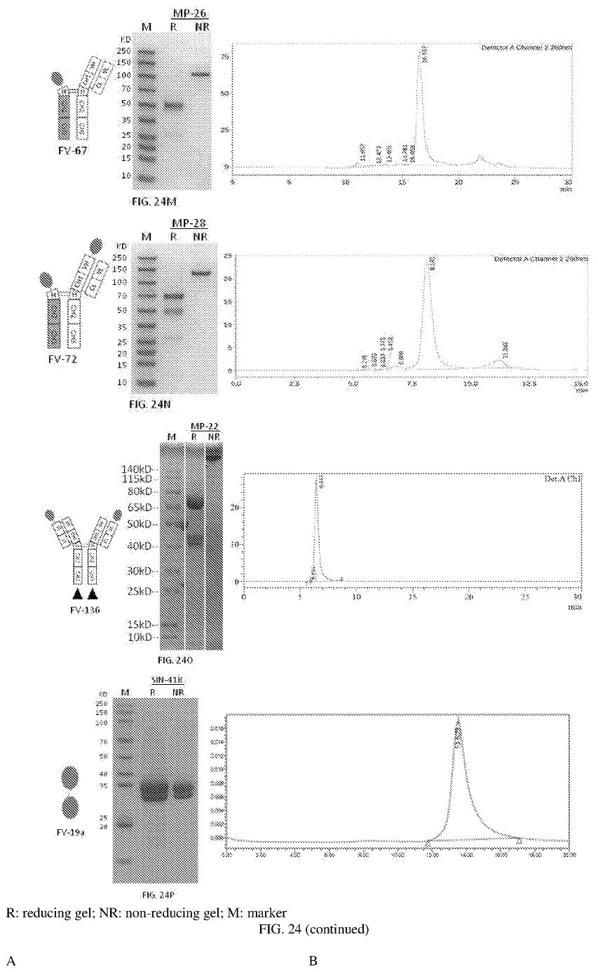

The results of representative proteins in FIG. 24 show that the various representative fusion proteins comprising at least one SIRP IgV monomer were produced at high purity (generally over 90% purity in SEC-HPLC assay) through a single-step purification, despite the wide variety of valency, formats and compositions, thus demonstrating broad compatibility of the SIRP IgV monomer for constructing fusion proteins of various valencies, formats and compositions. Notably, representative proteins comprising standalone SIRP IgV multimer polypeptide fusing two SIRP IgV monomers in tandem without Fc or other non SIRP IgV domain were also produced generally with good yield and purity (FIG. 24P and Table 22).

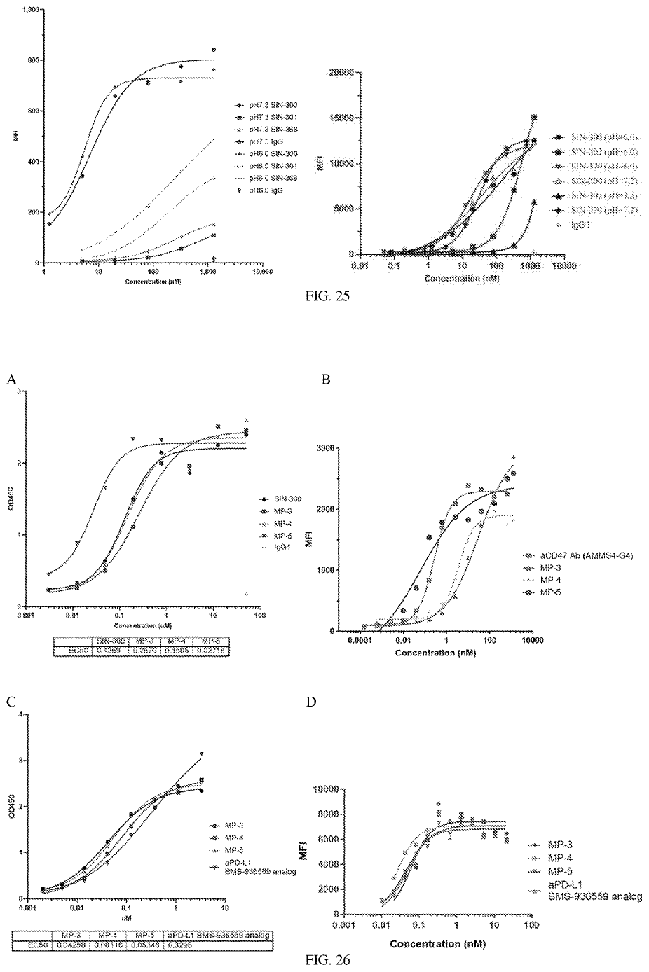

The CD47-binding of the produced proteins at both acidic and physiological pH are then analyzed through ELISA and FACS binding assays, as similarly done as described in the General Methods and the Examples above, and compared among proteins having the same valency of SIPR IgV monomers but different mutations, and among proteins having the same mutations of SIRP IgV monomers but different valencies. Similar to the results of the different mutations of SIRP IgV in the bivalent SIRP IgV-IgG1 Fe fusion protein as configured in format variant FV-2 in Examples above showing that SIRP IgV specifically with the mutation of K53H, R69H, Q52H or K68H, but not with the mutation of V33H, M72H or G97H, exhibited higher binding to CD47 at an acidic pH than at physiological pH, SIRP IgV specifically with the mutation of K53H, R69H, Q52H or K68H, but not with the mutation of V33H, M72H or G97H, exhibited higher binding to CD47 at an acidic pH than at physiological pH in the standalone bivalent SIRP IgV multimer polypeptide proteins linking two SIRP IgV monomers in tandem and also in fusion proteins of various format variants as exemplified in FIG. 24 with monovalent to hexavalent SIRP IgV monomers. Also, SIRP IgV with the mutation of K53H generally exhibited stronger binding than that with the mutation of R69H configured in the same format and valency at both acidic and physiological pH (FIG. 27A, 28A-C). For SIRP IgV fusion proteins with the same SIRP IgV mutation (e.g. single mutation of K53H) but different valency, however surprisingly, it's found that tetravalency comprising four SIRP IgV monomers with the same mutation actually showed generally better pH-differential binding at an acidic pH and physiological pH than higher hexa valency comprising six SIRP IgV monomers with the same mutation (FIG. 27B, 28A). Specifically, the Raji FACS assay result in FIG. 25A shows that a tetravalent SIRPαV2 IgV K53H-IgG1 Fc fusion protein SIN-368, configured in format FV-3 comprising a SIRP IgV multimer polypeptide (SEQ ID NO: 418) comprising two SIRPαV2 IgV monomers with the same single mutation of K53H directly fused to the N-terminal of hIgG1 Fc (SEQ ID NO: 135), exhibited increased binding than the bivalent SIRPαV2 IgV K53H-IgG1 Fe counterpart protein SIN-301 at both acidic pH 6.0 and physiological pH 7.3. Notably, the increase shown at an acidic pH was larger than that at physiological pH, thus favorably expanding the binding difference at an acidic pH versus at physiological pH. Meanwhile SIN-370, a hexavalent SIRP IgV fusion protein comprising a trimer of three bivalent SIRPαV2 IgV R69H-IgG1 Fc-CD40L fusion protein configured in format FV-114 (SEQ ID NO: 370) also exhibited increased binding than the bivalent SIRPαV2 IgV R69H-IgG1 Fc counterpart protein SIN-302 at both acidic pH 6.0 and physiological pH 7.3, but the increase shown at an acidic pH was however smaller than that at physiological pH, thus unfavorably shrinking the binding difference at an acidic pH versus at physiological pH and also unfavorably making the protein SIN-370 binding to CD47 as strongly as the benchmark bivalent SIRPαV2 IgV WT-IgG1 Fc fusion protein SIN-300 at physiological pH (FIG. 25B).

In addition to the Fc-fusion proteins comprising tetravalent or hexavalent SIRP IgV monomers linked to the N-terminal of Fc, additional formats of antibody fusion proteins comprising tetravalent and hexavalent SIRP IgV monomers linked to the N-terminal of light chain and/or heavy chain of an antibody were also analyzed.

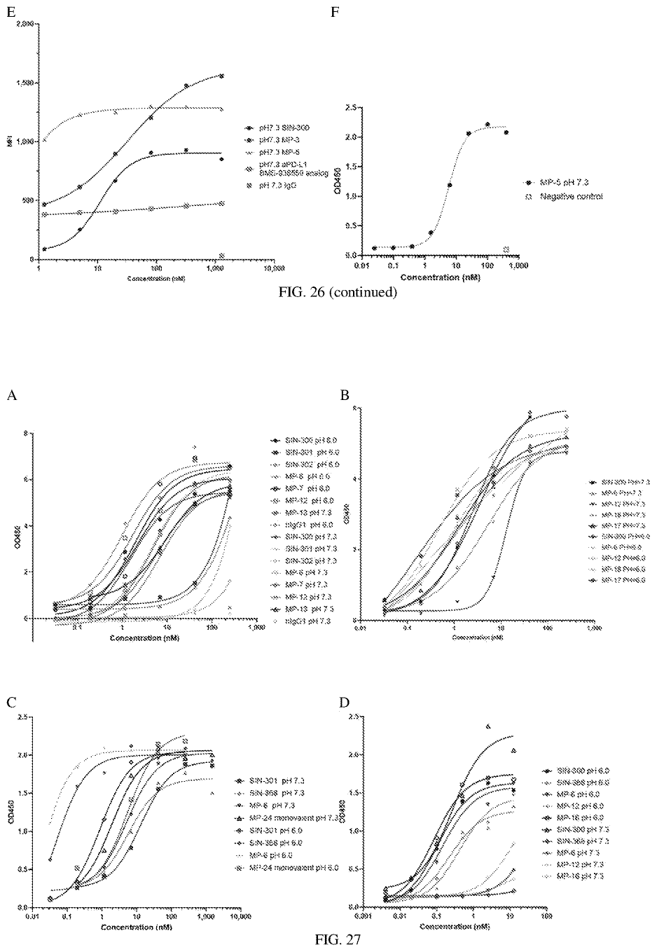

Particularly in one format, SIRP IgV monomers are fused to the N-terminal of both heavy chain and light chain, as illustrated in FV-54. However, fusing an extra domain to the N-terminal of both VH (heavy chain) and VL (light chain) of the antibody Fab domain may block or significantly impair the binding of the antibody Fab domain to its antigen, and/or cause poor expression and/or biophysical properties of the antibody fusion protein. To test this, an exemplary tetravalent antibody fusion protein (MP-5) fusing a SIRPαV2 IgV WT monomer to the N-terminal of both light chain and heavy chain of an exemplary anti-PD-L1 antibody BMS-936559 through a (GGGGS) 4 linker was constructed and expressed in HEK293 cells. Surprisingly, MP-S was well expressed with good yield (42.7 mg/L) and high purity through a single step Protein A purification, comparable to the proteins MP-3 and MP-4, wherein the same SIRPαV2 IgV WT monomer is fused to the N-terminal of only the light chain or the heavy chain respectively of the same antibody BMS-936559 through the same (GGGGS) 4 linker (FIG. 24E-G). Importantly, ELISA result in FIG. 26A and Raji FACS assay result in FIG. 26B show that the tetravalent MP-5 protein exhibited largely increased binding to CD47 than the bivalent counterparts MP-3 and MP-4 which showed comparable binding to the benchmark bivalent SIRPαV2 IgV WT-IgG1 Fc fusion protein SIN-300. Notably in FIG. 26B, MP-5 showed comparable binding to Raji cells as the anti-CD47 antibody AMMS4-G4, which reports similar binding to Hu5F9-G4 (magrolimab) (Yu et al., Biochimie, 2018). Surprisingly, the ELISA result in FIG. 26C and the FACS assay result on MC38-hPD-L1 cells (mouse MC38 cell line with human PD-L1 overexpression but no human CD47 expression) in FIG. 26D show that the MP-5 protein exhibited binding to PD-L1 at a level comparable, if not higher, to that of the parent anti-PD-L1 antibody BMS-936559 analog as well as to that of the bivalent fusion protein MP-3 and MP-4. These results thus show that the fusion of a SIRPαV2 IgV domain to the N-terminal of both the light chain and heavy chain surprisingly doesn't block or impair the binding of the antibody Fab domain to its target antigen. Additionally, the result in FIG. 26E from FACS assay of the HT-1080 human fibrosarcoma cells which express both human CD47 and PD-L1 shows that the protein MP-5 and MP-3 both exhibited higher binding than either the parent aPD-L1 antibody BMS-936559 analog alone or bivalent SIRPαV2 IgV WT-IgG1 Fe fusion protein SIN-300 alone, demonstrating simultaneous binding to PD-L1 and CD47 by the antibody fusion protein. Also at lower concentrations before reaching saturating binding plateau, MP-5 exhibited much higher binding than that of MP-3 and also the sum of the binding of anti-PD-L1 BMS-936559 alone and anti-CD47 SIN-300 alone (FIG. 26E), demonstrating effect of increased tetravalent avidity binding. The simultaneous binding to PD-L1 and CD47 by MP-5 was further directly confirmed by ELISA, as shown in the result of FIG. 26F. For this ELISA to detect simultaneous binding to PD-L1 and CD47, human PD-L1 protein (ACRO Biosystems Cata #PD1-H5282) was immobilized to 96-well plate and MP-5 at serial dilutions was used as the 1st analyte to bind to the immobilized PD-L1. After washing away of unbound MP-5, a His-tag human CD47 ECD protein was then used as the 2nd analyte to probe the MP-5 bound to the immobilized PD-L1, and the binding was finally detected through anti-His-HRP after washing.

Similar to the protein MP-S above, antibody fusion proteins configured in the format FV-54 by fusing a variant SIRP IgV domain to the N-terminal of both the VL and VH of an antibody or Fab domain can be constructed. The SIRP IgV monomers fused to the N-terminal of the VL and VH chain can comprise the same or different mutation. For example, such formatted anti-PD-L1 antibody (BMS-936559) fusion protein comprising tetravalent SIRPαV2 IgV monomers with homogeneous mutation of K53H (protein MP-6), R69H (protein MP-7), Q52H (protein MP-10) or K68H (protein MP-11), or a mixture of two SIRPαV2 IgV monomers comprising R69H mutation pairing with two SIRPαV2 IgV monomers comprising Q52H (e.g. protein MP-8), or K68H (e.g. protein MP-9) mutation can be constructed and well produced at high purity, as shown in Table 13, Table 20 and FIG. 24H. Additionally, antibody fusion proteins configured in the format FV-5 (FIG. 2) by fusing a SIRP IgV domain to the N-terminal of a partial heavy chain comprising CH1-CH2-CH3 without the VH domain and the N-terminal of a CL domain without the VL domain, can also be constructed as shown in Table 12 and Table 19. Despite lack of the VH and VL domain, such SIRP IgV-antibody fusion protein configured in format FV-5, for example SP-25, surprisingly can be well produced at very high purity (100% SEC-HPLC purity) and very high yield (287 mg/L), thus enabling generation of such monospecific tetravalent SIRP IgV antibody fusion protein configured in format FV-5 with increased binding avidity effect similar to the SIRP IgV antibody fusion protein configured in format FV-54. Similarly, antibody fusion proteins configured in the format FV-4 or FV8 (FIG. 2) by fusing a SIRP IgV domain to the N-terminal of CL domain only but not to the partial heavy chain comprising CH1-CH2-CH3, can also be constructed as shown in Table 12 and Table 19 and well produced.

Meanwhile, similar to the tetravalent protein SIN-368 shown above, antibody fusion proteins configured in the format FV-50 or FV-51 by fusing a SIRP IgV multimer polypeptide comprising two tandem SIRP IgV monomers (e.g. comprising a sequence of SEQ ID NO: 82 to 96 and SEQ ID NO: 418 to 425) to the N-terminal of only the VL or VH of an antibody or Fab domain can be constructed, as depicted in Table 13. For example, such formatted anti-PD-L1 antibody fusion protein comprising tetravalent SIRPαV2 IgV monomers with homogeneous mutation of K53H (protein MP-12), R69H (protein MP-13), Q52H or K68H, or a mixture of two SIRPαV2 IgV monomers comprising R69H mutation pairing with two SIRPαV2 IgV monomers comprising Q52H (e.g. protein MP-15), or K68H (e.g. protein MP-14) can be constructed and well produced at high purity, as shown in Table 13, Table 20 and FIG. 24I. In addition to the exemplary anti-PD-L1 antibody, such formatted fusion proteins with additional exemplary antibodies such as daratumumab (e.g. protein MP-20) and cetuximab (e.g. protein MP-21) can also be constructed and well produced at high purity, as shown in Table 13, Table 20 and FIG. 24J.

What's more, additional mutation of N80A, N80S or N80Q can be introduced to the SIRP IgV domain to abolish its glycosylation and reduce the molecular weight of the fusion protein. As an example, an additional mutation of N80A was introduced into the variant SIRPαV2 IgV domain of the protein MP-6 to generate protein MP-16 to abolish glycosylation of the SIRPαV2 IgV domain for better homogeneity of the protein production and reduce the molecule weight of the fusion protein. As shown in the result of FIG. 24H, MP-16 exhibits one single <50KD band under reducing SDS-PAGE versus two close <50KD bands shown by MP-6 which is due to heterogeneity of glycosylation of the SIRPαV2 IgV domain in the SIRPαV2 IgV-light chain fusion polypeptide. The overall molecular weight of MP-16 shown under non-reducing SDS-PAGE is also lower than that of MP-6, consistent with deglycosylation of the SIRPαV2 IgV domains.

In addition to tetravalent fusion proteins, fusion proteins comprising hexavalent SIRP IgV monomers can also be constructed in various formats. In the format FV-55 or 56, a SIRP IgV multimer comprising two tandem SIRP IgV monomers is fused to the N-terminal of VL chain and one SIRP IgV monomer is fused to N-terminal of the VH chain, or vice versa, wherein the SIRP IgV monomers can comprise the same or different mutation. For example, anti-PD-L1 antibody fusion protein comprising hexavalent SIRPαV2 IgV monomers configured in format FV-55 with homogenous mutation of K53H (protein MP-18) or R69H (protein MP-19) can be constructed and well produced, as shown in Table 13, Table 20 and FIG. 24L. Similarly, in another format FV-52 or 53, a SIRP IgV multimer polypeptide comprising three SIRP IgV monomers (e.g. comprising a sequence of SEQ ID NO: 97 to 109 and SEQ ID NO: 430 to 435) is fused to the N-terminal of VL or VH, wherein the SIRP IgV monomers can comprise the same or different mutation. Exemplary anti-PD-L1 antibody fusion protein comprising hexavalent SIRPαV2 IgV monomers configured in format FV-52 with homogenous mutation of K53H or K53H+N80A combo mutation (protein MP-17) can be constructed and well produced at high purity, as shown in Table 13, Table 20 and FIG. 24K.

As summarized earlier, despite configured in different formats and valency of SIRP IgV monomers, the fusion proteins with SIRP IgV specifically comprising the mutation of K53H, R69H, Q52H or K68H exhibited higher binding to CD47 at an acidic pH than at physiological pH (FIG. 25A-B, 27A-D, 28A-C). Notably, SIRP IgV with the mutation of K53H generally exhibited stronger binding than that with the mutation of R69H configured in the same format and valency at both acidic and physiological pH (FIG. 27A, 28A-C). Meanwhile, for the fusion proteins with SIRP IgV comprising the same mutation (e.g. single mutation of K53H or R69H) but at different valency, the ELISA results in FIG. 27A-D show that the fusion proteins comprising tetravalent SIRP IgV monomers with K53H mutation (SIN-368, MP-6, MP-12) or R69H mutation (MP-7, MP-13) exhibited increased CD47-binding at both acidic and physiological pH than the fusion protein comprising bivalent SIRP IgV monomers with the same K53H mutation (SIN-301) or R69H mutation (SIN-302), and the increase at an acidic pH was generally larger or similar than the increase at physiological pH so that the tetravalent fusion protein exhibited a generally larger or similar CD47-binding difference at an acidic pH and physiological pH than that of the bivalent fusion protein (FIG. 27A, 27C). Meanwhile, the fusion protein comprising hexavalent SIRP IgV monomers with K53H mutation configured in different formats, namely MP-17 configured in format FV-52 and MP-18 configured in format FV-55, exhibited further increased CD47-binding at both acidic and physiological pH than the tetravalent protein with the same SIRP IgV mutation (MP-6, MP-12), but surprisingly the increase at physiological pH was actually larger than the increase at an acidic pH so that the hexavalent protein actually exhibited a unfavorably smaller binding difference at the acidic and physiological pH and also unfavorably high binding at physiological pH similar to that of the benchmark protein SIN-300 (analog to clinically tested TTI-621) comprising wild type SIRP IgV (FIG. 27B). Thus when higher binding at an acidic pH is sought, tetravelency but not the higher hexavalency was unexpectedly identified as the better valency for more favorable pH-differential binding at an acidic pH and physiological pH. Meanwhile within tetravalency, the tetravalent fusion protein comprising four SIRP IgV monomers separately linked to the N-terminal of the VH and VL of the antibody as illustrated in format FV-54, exhibited generally higher CD47-binding at acidic and/or physiological pH than the fusion protein comprising four SIRP IgV monomers with the same mutation wherein two monomers were serially linked together, for example the MP-6 versus MP-12 comprising the same mutation of K53H, and the protein MP-7 versus MP-13 comprising the same mutation of R69H (FIG. 27A, 27B, 27D). Additionally, the result in FIG. 27D shows that deglycosylation of the SIRP IgV through introduction of N80A mutation also increased CD47-binding at both acidic and physiological pH, as the protein MP-16 exhibited higher binding than the otherwise identical protein MP-6 except without addition of the N80A mutation.

TABLE 12
Assembly and composition of exemplary monospecific Fc-fusion proteins with a SIRP IgV domain

| | Fc | | | | | Linker |
|---|---|---|---|---|---|---|
| Format | Exemplary Fc | SEQ ID NO: of | SEQ ID NO: of exemplary SIRP | CH1 (SEQ | CL (SEQ | SEQ ID |
| variant | type | exemplary Fc | IgV domain | ID NO:) | ID NO:) | NO: |
| FV-1 | Heterodimeric | SEQ ID NO: 136 × | SEQ ID NO: 47 × 59, 23 × 59, 35 × | — | — | — |
| | hIgG1 Fc | 137 (the “×” | 49, 47 × 23, 47 × 35 (the “×” | | | |
| | | indicates the | indicates the SEQ ID NO: of a | | | |
| | | SEQ ID NO: of a | pair of SIRP IgV monomers | | | |
| | | pair of | separately linked to the N- | | | |
| | | heterodimeric Fc | terminal of the two | | | |
| | | chains in FV-1) | heterodimeric Fc chains in FV-1) | | | |
| FV-2 | Homodimeric | SEQ ID NO: 135 | SEQ ID NO: 3 to 81 | — | — | — |
| | hIgG1 Fc | | | | | |
| FV-2 | Homodimeric | SEQ ID NO: 130 | SEQ ID NO: 3 to 21 | — | — | — |
| | hIgG4 Fc | | | | | |
| FV-3 | homodimeric | SEQ ID NO: 135 | SEQ ID NO: 82 to 96, 418 to 429 | — | — | — |
| | hIgG1 Fc | | | | | |
| FV-3 | homodimeric | SEQ ID NO: 130 | SEQ ID NO: 3 to 21, 429 | — | — | SEQ ID |
| | hIgG4 Fc | | | | | NO: 121 |
| FV-4 | homodimeric | SEQ ID NO: 135 | SEQ ID NO: 82 to 96, 418 to 429 | SEQ ID | SEQID | SEQ ID |
| | hIgG1 Fc | | | NO: 128 | NO: 124 | NO: 121 |
| FV-4 | homodimeric | SEQ ID NO: 130 | SEQ ID NO: 3 to 21, 429 | SEQ ID | SEQ ID | SEQ ID |
| | hIgG4 Fc | | | NO: 126 | NO: 124 | NO: 121 |
| FV-8 | homodimeric | SEQ ID NO: 135 | SEQ ID NO: 3 to 81 | SEQ ID | SEQ ID | SEQ ID |
| | hIgG1 Fc | | | NO: 128 | NO: 124 | NO: 121 |
| FV-8 | homodimeric | SEQ ID NO: 130 | SEQ ID NO: 3 to 21 | SEQ ID | SEQ ID | SEQ ID |
| | hIgG4 Fc | | | NO: 126 | NO: 124 | NO: 121 |
| FV-5 | homodimeric | SEQ ID NO: 135 | SEQ ID NO: 47 × 47, 59 × 59, 47 × | SEQ ID | SEQ ID | SEQ ID |
| | hIgG1 Fc | | 59, 71 × 59, 71 × 47 (the “×” | NO: 128 | NO: 124 | NO: 121 |
| | | | indicates the SEQ ID NO: of a | | | |
| | | | pair of SIRP IgV monomers | | | |
| | | | separately linked to the N- | | | |
| | | | terminal of the CH1 chain and | | | |
| | | | CL chain or vice versa in FV-5) | | | |
| FV-5 | homodimeric | SEQ ID NO: 130 | SEQ ID NO: 3 to 21 | SEQ ID | SEQ ID | SEQ ID |
| | hIgG4 Fc | | | NO: 126 | NO: 124 | NO: 121 |
| FV-6 | homodimeric | SEQ ID NO: 135 | SEQ ID NO: 97 to 109, 430 to | — | — | — |
| | hIgG1 Fc | | 439 | | | |
| FV-6 | homodimeric | SEQ ID NO: 130 | SEQ ID NO: 3 to 21, 439 | — | — | SEQ ID |
| | hIgG4 Fc | | | | | NO: 121 |
| FV-9 to | homodimeric | SEQ ID NO: 135 | SEQ ID NO: 22 to 113 | — | — | SEQ ID |
| 14 | hIgG1 Fc | | | | | NO: 121 |

TABLE 13
Assembly and composition of exemplary homodimeric multispecific antibody fusion proteins comprising a SIRP IgV domain

| | Fc | | | | | | |
|---|---|---|---|---|---|---|---|
| | | SEQ ID | | | | | |
| | | NO: of | SEQ ID NO: of | | CH1 | | Linker |
| Format | Exemplary | exemplary | exemplary SIRP | Exemplary VH × VL | (SEQ ID | CL (SEQ | SEQ ID |
| variant | Fc type | Fc | IgV domain | pair (SEQ ID NO:) | NO:) | ID NO:) | NO: |
| FV-48 | Homo- | SEQ ID | SEQ ID NO: 3 to 81 | SEQ ID NO: 144 × 145, 146 × 147, | SEQ ID | SEQ ID | SEQ ID |
| or 49 | dimeric | NO: 135 | | 148 × 149, 150 × 151, 152 × 153, 154 × 155, | NO: 128 | NO: | NO: 121 |
| | hIgG1 Fc | | | 156 × 157, 158 × 159, 160 × 161, 162 × 163, | | 124 or | |
| | | | | 164 × 165, 166 × 167, 168 × 169, 170 × 171, | | 125 | |
| | | | | 172 × 173, 174 × 175, 176 × 177, 178 × 179, | | | |
| | | | | 180 × 181, 182 × 183, 184 × 185, 186 × 187, | | | |
| | | | | 188 × 189, 190 × 191, 192 × 193, 194 × 195 | | | |
| FV-50 | Homo- | SEQ ID | SEQ ID NO: 82 to | SEQ ID NO: 144 × 145, 146 × 147, | SEQ ID | SEQ ID | SEQ ID |
| or 51 | dimeric | NO: 135 | 96, 418 to 429 | 148 × 149, 150 × 151, 152 × 153, 154 × 155, | NO: 128 | NO: | NO: 121 |
| | hIgG1 Fc | | | 156 × 157, 158 × 159, 160 × 161, 162 × 163, | | 124 or | |
| | | | | 164 × 165, 166 × 167, 168 × 169, 170 × 171, | | 125 | |
| | | | | 172 × 173, 174 × 175, 176 × 177, 178 × 179, | | | |
| | | | | 180 × 181, 182 × 183, 184 × 185, 186 × 187, | | | |
| | | | | 188 × 189, 190 × 191, 192 × 193, 194 × 195 | | | |
| FV-52 | Homo- | SEQ ID | SEQ ID NO: 97 to | SEQ ID NO: 144 × 145, 146 × 147, | SEQ ID | SEQ ID | SEQ ID |
| or 53 | dimeric | NO: 135 | 109, 430 to 439 | 148 × 149, 150 × 151, 152 × 153, 154 × 155, | NO: 128 | NO: | NO: 121 |
| | hIgG1 Fc | | | 156 × 157, 158 × 159, 160 × 161, 162 × 163, | | 124 or | |
| | | | | 164 × 165, 166 × 167, 168 × 169, 170 × 171, | | 125 | |
| | | | | 172 × 173, 174 × 175, 176 × 177, 178 × 179, | | | |
| | | | | 180 × 181, 182 × 183, 184 × 185, 186 × 187, | | | |
| | | | | 188 × 189, 190 × 191, 192 × 193, 194 × 195 | | | |
| FV-54 | Homo- | SEQ ID | SEQ ID NO: 3 to 81 × | SEQ ID NO: 144 × 145, 146 × 147, | SEQ ID | SEQ ID | SEQ ID |
| | dimeric | NO: 135 | SEQ ID NO: 3 to | 148 × 149, 150 × 151, 152 × 153, 154 × 155, | NO: 128 | NO: | NO: 121 |
| | hIgG1 Fc | | 81; e.g. SEQ ID NO: | 156 × 157, 158 × 159, 160 × 161, 162 × 163, | | 124 or | |
| | | | 47 × 47, 59 × 59, 47 × | 164 × 165, 166 × 167, 168 × 169, 170 × 171, | | 125 | |
| | | | 59, 71 × 59, 71 × | 172 × 173, 174 × 175, 176 × 177, 178 × 179, | | | |
| | | | 47 (“×” indicates | 180 × 181, 182 × 183, 184 × 185, 186 × 187, | | | |
| | | | SIRP IgV separately | 188 × 189, 190 × 191, 192 × 193, 194 × 195 | | | |
| | | | linked to VH × VL | | | | |
| | | | or VL × VH ) | | | | |
| FV-55 | Homo- | SEQ ID | SEQ ID NO: one of | SEQ ID NO: 144 × 145, 146 × 147, | SEQ ID | SEQ ID | SEQ ID |
| or 56 | dimeric | NO: 135 | SEQ ID NO: 46 to | 148 × 149, 150 × 151, 152 × 153, 154 × 155, | NO: 128 | NO: | NO: 121 |
| | hIgG1 Fc | | 69 × one of SEQ ID | 156 × 157, 158 × 159, 160 × 161, 162 × 163, | | 124 or | |
| | | | NO: 82 to 96 and | 164 × 165, 166 × 167, 168 × 169, 170 × 171, | | 125 | |
| | | | 418 to 429 (“×” | 172 × 173, 174 × 175, 176 × 177, 178 × 179, | | | |
| | | | indicates SIRP IgV | 180 × 181, 182 × 183, 184 × 185, 186 × 187, | | | |
| | | | separately linked | 188 × 189, 190 × 191, 192 × 193, 194 × 195 | | | |
| | | | to VH × VL or VL × | | | | |
| | | | VH) | | | | |

TABLE 14
Assembly and composition of exemplary heterodimeric multispecific antibody fusion protein comprising a SIRP IgV domain

| | Fc | | | | | | |
|---|---|---|---|---|---|---|---|
| | | SEQ ID | SEQ ID NO: of | | | | |
| | | NO: of | exemplary | | | | Linker |
| Format | Exemplary Fc | exemplary | SIRP IgV | | CH1 (SEQ | CL (SEQ | SEQ ID |
| variant | type | Fc | domain | Exemplary VH × VL pair (SEQ ID NO:) | ID NO:) | ID NO:) | NO: |
| FV-64, | Hetero - | SEQ ID | SEQ ID NO: 3 | SEQ ID NO: 144 × 145, 146 × 147, 148 × 149, | SEQ ID | SEQ ID | SEQ ID |
| 65 or 66 | dimeric | NO: 136 × | to 81 | 150 × 151, 152 × 153, 154 × 155, 156 × 157, 158 × 159, | NO: 128 | NO: 124 | NO: |
| | hIgG1 Fc | 137 (knob × | | 160 × 161, 162 × 163, 164 × 165, 166 × 167, 168 × 169, | | or 125 | 121 |
| | | hole) | | 170 × 171, 172 × 173, 174 × 175, 176 × 177, 178 × 179, | | | |
| | | | | 180 × 181, 182 × 183, 184 × 185, 186 × 187, 188 × 189, | | | |
| | | | | 190 × 191, 192 × 193, 194 × 195 | | | |
| FV-67 | Hetero - | SEQ ID | SEQ ID NO: 3 | SEQ ID NO: 144 × 145, 146 × 147, 148 × 149 | SEQ ID | SEQ ID | — |
| | dimeric | NO: 136 × | to 81 | 150 × 151, 152 × 153, 154 × 155, 156 × 157, 158 × 159, | NO: 128 | NO: 124 | |
| | hIgG1 Fc | 137 | | 160 × 161, 162 × 163, 164 × 165, 166 × 167, 168 × 169, | | or 125 | |
| | | | | 170 × 171, 172 × 173, 174 × 175, 176 × 177, 178 × 179, | | | |
| | | | | 180 × 181, 182 × 183, 184 × 185, 186 × 187, 188 × 189, | | | |
| | | | | 190 × 191, 192 × 193, 194 × 195, 196 × 197, 198 × 199, | | | |
| | | | | 200 × 201, 202 × 203, 204 × 205, 206 × 207, 208 × 209, | | | |
| | | | | 210 × 211, 212 × 213 | | | |
| FV-68 | Hetero - | SEQ ID | SEQ ID NO: | SEQ ID NO: 144 × 145, 145 × 147, 148 × 149, | SEQ ID | SEQ ID | SEQ ID |
| | dimeric | NO: 136 × | 82 to 96, 418 | 150 × 151, 152 × 153, 154 × 155, 156 × 157, 158 × 159, | NO: 128 | NO: 124 | NO: |
| | hIgG1 Fc | 137 | to 429 | 160 × 161, 162 × 163, 164 × 165, 166 × 167, 168 × 169, | | or 125 | 121 |
| | | | | 170 × 171, 172 × 173, 174 × 175, 176 × 177, 178 × 179, | | | |
| | | | | 180 × 181, 182 × 183, 184 × 185, 186 × 187, 188 × 189, | | | |
| | | | | 190 × 191, 192 × 193, 194 × 195, 196 × 197, 198 × 199, | | | |
| | | | | 200 × 201, 202 × 203, 204 × 205, 206 × 207, 208 × 209, | | | |
| | | | | 210 × 211, 212 × 213 | | | |
| FV-69 | Hetero - | SEQ ID | SEQ ID NO: | SEQ ID NO: 144 × 145, 145 × 147, 148 × 149, | SEQ ID | SEQ ID | SEQ ID |
| | dimeric | NO: 136 × | 97 to 109, | 150 × 151, 152 × 153, 154 × 155, 156 × 157, 158 × 159, | NO: 128 | NO: 124 | NO: |
| | hIgG1 Fc | 137 | 430 to 439 | 160 × 161, 162 × 163, 164 × 155, 166 × 167, 168 × 169, | | or 125 | 121 |
| | | | | 170 × 171, 172 × 173, 174 × 175, 176 × 177, 178 × 179, | | | |
| | | | | 180 × 181, 182 × 183, 184 × 185, 186 × 187, 188 × 189, | | | |
| | | | | 190 × 191, 192 × 193, 194 × 195, 196 × 197, 198 × 199, | | | |
| | | | | 200 × 201, 202 × 203, 204 × 205, 206 × 207, 208 × 209, | | | |
| | | | | 210 × 211, 212 × 213 | | | |
| FV-70 | Hetero - | SEQ ID | SEQ ID NO: | SEQ ID NO: 144 × 145, 145 × 147, 148 × 149, | SEQ ID | SEQ ID | SEQ ID |
| | dimeric | NO: 136 × | 110 to 113 | 150 × 151, 152 × 153, 154 × 155, 156 × 157, 158 × 159, | NO: 128 | NO: 124 | NO: |
| | hIgG1 Fc | 137 | | 160 × 161, 162 × 163, 164 × 165, 166 × 167, 168 × 169, | | or 125 | 121 |
| | | | | 170 × 171, 172 × 173, 174 × 175, 176 × 177, 178 × 179, | | | |
| | | | | 180 × 181, 182 × 183, 184 × 185, 186 × 187, 188 × 189, | | | |
| | | | | 190 × 191, 192 × 193, 194 × 195, 196 × 197, 198 × 199, | | | |
| | | | | 200 × 201, 202 × 203, 204 × 205, 206 × 207, 208 × 209, | | | |
| | | | | 210 × 211, 212 × 213 | | | |
| FV-71 or | Hetero - | SEQ ID | SEQ ID NO: 3 | SEQ ID NO: 144 × 145, 145 × 147, 148 × 149, | SEQ ID | SEQ ID | SEQ ID |
| 72 | dimeric | NO: 136 × | to 81 | 150 × 151, 152 × 153, 154 × 155, 156 × 157, 158 × 159, | NO: 128 | NO: 124 | NO: |
| | hIgG1 Fc | 137 | | 160 × 161, 162 × 163, 164 × 165, 166 × 167, 168 × 169, | | or 125 | 121 |
| | | | | 170 × 171, 172 × 173, 174 × 175, 176 × 177, 178 × 179, | | | |
| | | | | 180 × 181, 182 × 183, 184 × 185, 186 × 187, 188 × 189, | | | |
| | | | | 190 × 191, 192 × 193, 194 × 195, 196 × 197, 198 × 199, | | | |
| | | | | 200 × 201, 202 × 203, 204 × 205, 206 × 207, 208 × 209, | | | |
| | | | | 210 × 211, 212 × 213 | | | |
| FV-71 or | Hetero - | SEQ ID | SEQ ID NO: 3 | SEQ ID NO: 144 × 145, 146 × 147, 148 × 149, | SEQ ID | SEQ ID | SEQ ID |
| 72 | dimeric | NO: 136 × | to 21 (Fc | 150 × 151, 152 × 153, 154 × 155, 156 × 157, 158 × 159, | NO: 128 | NO: 124 | NO: |
| | hIgG1 Fc | 137 | chain SIRP | 160 × 161, 162 × 163, 164 × 165, 166 × 167, 168 × 169, | | or 125 | 121 |
| | | | IgV) × SEQ ID | 170 × 171, 172 × 173, 174 × 175, 176 × 177, 178 × 179, | | | |
| | | | NO: 58 to 69 | 180 × 181, 182 × 183, 184 × 185, 186 × 187, 188 × 189, | | | |
| | | | (LC or HC | 190 × 191, 192 × 193, 194 × 195, 196 × 197, 198 × 199, | | | |
| | | | SIRP IgV) in | 200 × 201, 202 × 203, 204 × 205, 206 × 207, 208 × 209, | | | |
| | | | FV-71 or 72 | 210 × 211, 212 × 213 | | | |
| FV-77 or | Hetero - | SEQ ID | SEQ ID NO: | SEQ ID NO: 144 × 145, 145 × 147, 148 × 149, | SEQ ID | SEQ ID | SEQ ID |
| 78 | dimeric | NO: 136 × | 82 to 96, 418 | 150 × 151, 152 × 153, 154 × 155, 156 × 157, 158 × 159, | NO: 128 | NO: 124 | NO: |
| | hIgG1 Fc | 137 | to 429 | 160 × 161, 162 × 163, 164 × 165, 166 × 167, 168 × 169, | | or 125 | 121 |
| | | | | 170 × 171, 172 × 173, 174 × 175, 176 × 177, 178 × 179, | | | |
| | | | | 180 × 181, 182 × 183, 184 × 185, 186 × 187, 188 × 189, | | | |
| | | | | 190 × 191, 192 × 193, 194 × 195, 196 × 197, 198 × 199, | | | |
| | | | | 200 × 201, 202 × 203, 204 × 205, 206 × 207, 208 × 209, | | | |
| | | | | 210 × 211, 212 × 213 | | | |

TABLE 15
Assembly and composition of exemplary heterodimeric trispecific antibody fusion protein comprising a SIRP IgV domain

| | Fc | | SEQ ID | Exemplary Fab | | | | |
|---|---|---|---|---|---|---|---|---|
| | | SEQ ID | NO: of | domain 1 binding to | | CH1 | CL | Linker |
| | | NO: of | exemplary | non-CD47 target | Exemplary Fab domain 2 | (SEQ | (SEQ | SEQ |
| Format | Exemplary | exemplary | SIRP IgV | (SEQ ID NO: of VH1 × | binding to non-CD47 target | ID | ID | ID |
| variant | Fc type | Fc | domain | VL1 pair) | (SEQ ID NO: of VH2 × VL2 pair) | NO:) | NO:) | NO: |
| FV-128 | Hetero- | SEQ ID | SEQ ID | SEQ ID NO: | SEQ ID NO: 144 × 145, 146 × 147, | SEQ | SEQ | SEQ |
| or 132 | dimeric | NO: 136 × | NO: 3 to | 190 × 191, 192 × 193, | 148 × 149, 150 × 151, 152 × 153, | ID | ID | ID |
| | hIgG1 Fc | 137 | 81 | 194 × 195, 196 × 197, | 154 × 155, 156 × 157, 158 × 159, | NO: | NO: | NO: |
| | | | | 198 × 199, 200 × 201, | 160 × 161, 162 × 163, 164 × 165, | 128 | 124 | 119 or |
| | | | | 202 × 203, 204 × 205, | 166 × 167, 168 × 169, 170 × 171, | | or | 121 |
| | | | | 206 × 207, 208 × 209, | 172 × 173, 174 × 175, 176 × 177, | | 125 | |
| | | | | 210 × 211, 212 × 213 | 178 × 179, 180 × 181, 182 × 183, | | | |
| | | | | | 184 × 185 | | | |
| FV-128 | Hetero- | SEQ ID | SEQ ID | SEQ ID NO: | SEQ ID NO: 164 × 165, 166 × 167, | SEQ | SEQ | SEQ |
| or 132 | dimeric | NO: 136 × | NO: 3 to | 144 × 145, 146 × 147, | 170 × 171, 172 × 173, 174 × 175, | ID | ID | ID |
| | hIgG1 Fc | 137 | 81 | 148 × 149, 150 × 151, | 176 × 177 | NO: | NO: | NO: |
| | | | | 152 × 153, 154 × 155, | | 128 | 124 | 119 or |
| | | | | 156 × 157, 158 × 159, | | | or | 121 |
| | | | | 160 × 161, 162 × 163, | | | 125 | |
| | | | | 168 × 169, 176 × 177, | | | | |
| | | | | 184 × 185 | | | | |
| FV-129, | Hetero- | SEQ ID | SEQ ID | SEQ ID NO: | SEQ ID NO: 144 × 145, 146 × 147, | SEQ | SEQ | SEQ |
| 130 or | dimeric | NO: 136 × | NO: 82 to | 190 × 191, 192 × 193, | 148 × 149, 150 × 151, 152 × 153, | ID | ID | ID |
| 131 | hIgG1 Fc | 137 | 113, 418 | 194 × 195, 196 × 197, | 154 × 155, 156 × 157, 158 × 159, | NO: | NO: | NO: |
| | | | to 439 | 198 × 199, 200 × 201, | 160 × 161, 162 × 163, 164 × 165, | 128 | 124 | 119 or |
| | | | | 202 × 203, 204 × 205, | 166 × 167, 168 × 169, 170 × 171, | | or | 121 |
| | | | | 206 × 207, 208 × 209, | 172 × 173, 174 × 175, 176 × 177, | | 125 | |
| | | | | 210 × 211, 212 × 213 | 178 × 179, 180 × 181, 182 × 183, | | | |
| | | | | | 184 × 185 | | | |
| FV-129, | Hetero- | SEQ ID | SEQ ID | SEQ ID NO: | SEQ ID NO: 164 × 165, 166 × 167, | SEQ | SEO | SEQ |
| 130 or | dimeric | NO: 136 × | NO: 82 to | 144 × 145, 146 × 147, | 170 × 171, 172 × 173, 174 × 175, | ID | ID | ID |
| 131 | hIgG1 Fc | 137 | 113, 418 | 148 × 149, 150 × 151, | 176 × 177 | NO: | NO: | NO: |
| | | | to 439 | 152 × 153, 154 × 155, | | 128 | 124 | 119 or |
| | | | | 156 × 157, 158 × 159, | | | or | 121 |
| | | | | 160 × 161, 162 × 163, | | | 125 | |
| | | | | 168 × 169, 176 × 177, | | | | |
| | | | | 184 × 185 | | | | |
| FV-133, | Hetero- | SEQ ID | SEQ ID | SEQ ID NO: | SEQ ID NO: 144 × 145, 146 × 147, | SEQ | SEQ | SEQ |
| 134 or | dimeric | NO: 136 × | NO: 3 to | 190 × 191, 192 × 193, | 148 × 149, 150 × 151, 152 × 153, | ID | ID | ID |
| 135 | hIgG1 Fc | 137 | 113, 418 | 194 × 195, 196 × 197, | 154 × 155, 156 × 157, 158 × 159, | NO: | NO: | NO: |
| | | | to 439 | 198 × 199, 200 × 201, | 160 × 161, 162 × 163, 164 × 165, | 128 | 124 | 119 or |
| | | | | 202 × 203, 204 × 205, | 166 × 167, 168 × 169, 170 × 171, | | or | 121 |
| | | | | 206 × 207, 208 × 209, | 172 × 173, 174 × 175, 176 × 177, | | 125 | |
| | | | | 210 × 211, 212 × 213 | 178 × 179, 180 × 181, 182 × 183, | | | |
| | | | | | 184 × 185 | | | |
| FV-133, | Hetero- | SEQ ID | SEQ ID | SEQ ID NO: | SEQ ID NO: 164 × 165, 166 × 167, | SEQ | SEQ | SEQ |
| 134 or | dimeric | NO: 136 × | NO: 3 to | 144 × 145, 146 × 147, | 170 × 171, 172 × 173, 174 × 175, | ID | ID | ID |
| 135 | hIgG1 Fc | 137 | 113, 418 | 148 × 149, 150 × 151, | 176 × 177 | NO: | NO: | NO: |
| | | | to 439 | 152 × 153, 154 × 155, | | 128 | 124 | 119 or |
| | | | | 156 × 157, 158 × 159, | | | or | 121 |
| | | | | 160 × 161, 162 × 163, | | | 125 | |
| | | | | 168 × 169, 176 × 177, | | | | |
| | | | | 184 × 185 | | | | |

TABLE 16
Assembly and composition of Fab fusion protein comprising a SIRP IgV domain

| | SEQ ID NO: | | | | |
|---|---|---|---|---|---|
| | of exemplary | | | | Linker |
| Format | SIRP IgV | Exemplary VH x VL pair | CH1 (SEQ | CL (SEQ | SEQ ID |
| variant | domain | (SEQ ID NO:) | ID NO:) | ID NO:) | NO: |
| FV-29a, | SEQ ID NO: 3 | SEQ ID NO: 190x191, | SEQ ID | SEQ ID | SEQ ID |
| b, c or d | to 81 | 192x193, 194x195, 196x197, | NO: 126 | NO: 124 | NO: 121 |
| | | 198x199, 200x201, 202x203, | or 128 | or 125 | |
| | | 204x205, 206x207, 208x209 | | | |
| FV-30a, | SEQ ID NO: | SEQ ID NO: 190x191, | SEQ ID | SEQ ID | SEQ ID |
| b, c or d | 82 to 96, 418 | 192x193, 194x195, 196x197, | NO: 126 | NO: 124 | NO: 121 |
| | to 429 | 198x199, 200x201, 202x203, | or 128 | or 125 | |
| | | 204x205, 206x207, 208x209 | | | |
| FV-31a, | SEQ ID NO: | SEQ ID NO: 190x191, | SEQ ID | SEQ ID | SEQ ID |
| b, c or d | 97 to 109, | 192x193, 194x195, 196x197, | NO: 126 | NO: 124 | NO: 121 |
| | 430 to 439 | 198x199, 200x201, 202x203, | or 128 | or 125 | |
| | | 204x205, 206x207, 208x209 | | | |
| FV-32a, | SEQ ID NO: | SEQ ID NO: 190x191, | SEQ ID | SEQ ID | SEQ ID |
| b, cord | 110 to 113 | 192x193, 194x195, 196x197, | NO: 126 | NO: 124 | NO: 121 |
| | | 198x199, 200x201, 202x203, | or 128 | or 125 | |
| | | 204x205, 206x207, 208x209 | | | |
| FV-33a | SEQ ID NO: 3 | SEQ ID NO: 190x191, | SEQ ID | SEQ ID | SEQ ID |
| or b | to 81 | 192x193, 194x195, 196x197, | NO: 126 | NO: 124 | NO: 121 |
| | | 198x199, 200x201, 202x203, | or 128 | or 125 | |
| | | 204x205, 206x207, 208x209 | | | |
| FV-34a | SEQ ID NO: | SEQ ID NO: 190x191, | SEQ ID | SEQ ID | SEQ ID |
| or b | 82 to 96, 418 | 192x193, 194x195, 196x197, | NO: 126 | NO: 124 | NO: 121 |
| | to 429 | 198x199, 200x201, 202x203, | or 128 | or 125 | |
| | | 204x205, 206x207, 208x209 | | | |

TABLE 17
Assembly and composition of exemplary fusion protein comprising a SIRP IgV domain

| | Fc | | SEQ ID NO: | | |
|---|---|---|---|---|---|
| | | SEQ ID NO: | of | Exemplary peptide or | |
| | | of | exemplary | polypeptide binding | Linker |
| Format | Exemplary Fc | exemplary | SIRP IgV | to non-CD47 target | SEQ ID |
| variant | type | Fc | domain | (SEQ ID NO:) | NO: |
| FV-101 | Heterodimeric | SEQ ID NO: | SEQ ID NO: | SEQ ID NO: 221 to 241 | SEQ ID |
| | hIgG1 Fc | 136 x 137 | 3 to 81 | | NO: 121 |
| FV-102 | Heterodimeric | SEQ ID NO: | SEQ ID NO: | SEQ ID NO: 221 to 241 | SEQ ID |
| | hIgG1 Fc | 136 x 137 | 82 to 96, | | NO: 121 |
| | | | 418 to 429 | | |
| FV-103 | Heterodimeric | SEQ ID NO: | SEQ ID NO: | SEQ ID NO: 221 to 241 | SEQ ID |
| | hIgG1 Fc | 136 x 137 | 97 to 109, | | NO: 121 |
| | | | 430 to 439 | | |
| FV-104 | Heterodimeric | SEQ ID NO: | SEQ ID NO: | SEQ ID NO: 221 to 241 | SEQ ID |
| | hIgG1 Fc | 136 x 137 | 110 to 113 | | NO: 121 |
| FV-109 | Heterodimeric | SEQ ID NO: | SEQ ID NO: | SEQ ID NO: 221 to 241 | SEQ ID |
| | hIgG1 Fc | 136 x 137 | 3 to 81 | | NO: 121 |
| FV-111 | Heterodimeric | SEQ ID NO: | SEQ ID NO: | SEQ ID NO: 221 to 241 | SEQ ID |
| | hIgG1 Fc | 136 x 137 | 82 to 96, | | NO: 121 |
| | | | 418 to 429 | | |
| FV-114 | Homodimeric | SEQ ID NO: | SEQ ID NO: | SEQ ID NO: 242 to 253 | SEQ ID |
| | hIgG1 Fc | 135 | 46 to 69 | | NO: 121 |
| FV-19a | — | — | SEQ ID NO: | — | SEQ ID |
| to c | | | 82 to 113, | | NO: 121 |
| | | | 418 to 439 | | |
| FV-20a | — | — | SEQ ID NO: | SEQ ID NO: 242 to 253 | SEQ ID |
| or b | | | 3 to 81 | | NO: 121 |
| FV-21a | — | — | SEQ ID NO: | SEQ ID NO: 242 to 253 | SEQ ID |
| or b | | | 82 to 96, | | NO: 121 |
| | | | 418 to 429 | | |
| FV-27a | — | — | SEQ ID NO: | SEQ ID NO: 242 to 253 + | SEQ ID |
| to f | | | 3 to 81 | SEQ ID NO: 214 | NO: 121 |
| FV-28a | — | — | SEQ ID NO: | SEQ ID NO: 242 to 253 + | SEQ ID |
| to d | | | 82 to 96, | SEQ ID NO: 214 | NO: 121 |
| | | | 418 to 429 | | |

TABLE 18
Constructs of exemplary SIRP IgV multimer polypeptides

| Exemplary | | | Chain 1 |
|---|---|---|---|
| format | Protein | Description of exemplary | SEQ ID |
| variant | ID | protein | NO: |
| FV-19a | SIN-418 | SIRPαV2 IgV K53H-K53H | 418 |
| | SIN-419 | SIRPαV2 IgV R69H-R69H | 419 |
| | SIN-420 | SIRPαV2 IgV Q52H-Q52H | 420 |
| | SIN-421 | SIRPαV2 IgV K68H-K68H | 421 |
| | SIN-422 | SIRPαV2 IgV K53H-R69H | 422 |
| | SIN-423 | SIRPαV2 IgV R69H-K53H | 423 |
| | SIN-424 | SIRPαV2 IgV R69H-Q52H | 424 |
| | SIN-425 | SIRPαV2 IgV R69H-K68H | 425 |
| | SIN-426 | SIRPαV2 IgV M72H-M72H | 426 |
| | SIN-427 | SIRPαV2 IgV V33H-V33H | 427 |
| | SIN-428 | SIRPαV2 IgV G97H-G97H | 428 |
| | SIN-429 | SIRPαV2 IgV WT-WT | 429 |
| FV-19b | SIN-430 | SIRPαV2 IgV K53H-K53H-K53H | 430 |
| | SIN-431 | SIRPαV2 IgV R69H-R69H-R69H | 431 |
| | SIN-432 | SIRPαV2 IgV K53H-K53H-R69H | 432 |
| | SIN-433 | SIRPαV2 IgV R69H-K53H-K53H | 433 |
| | SIN-434 | SIRPαV2 IgV K53H-R69H-R69H | 434 |
| | SIN-435 | SIRPαV2 IgV R69H-R69H-K53H | 435 |
| | SIN-436 | SIRPαV2 IgV M72H-M72H-M72H | 436 |
| | SIN-437 | SIRPαV2 IgV V33H-V33H-V33H | 437 |
| | SIN-438 | SIRPαV2 IgV G97H-G97H-G97H | 438 |
| | SIN-439 | SIRPαV2 IgV WT-WT-WT | 439 |

TABLE 19
Constructs of representative SIRP IgV Fc-fusion proteins of exemplary formats

| Exemplary | | | Chain 1 | Chain 2 |
|---|---|---|---|---|
| format | | | SEQ ID | SEQ ID |
| variant | Protein ID | Description of representative protein | NO | NO: |
| FV-8 | SP-1 | SIRPαV2 IgV K53H-CL + CH1-CH2-CH3 IgG1 Fc | 440 | 454 |
| | SP-2 | SIRPαV2 IgV R69H-CL + CH1-CH2-CH3 IgG1 Fc | 441 | 454 |
| | SP-3 | SIRPαV2 IgV Q52H-CL + CH1-CH2-CH3 IgG1 Fc | 442 | 454 |
| | SP-4 | SIRPαV2 IgV K68H-CL + CH1-CH2-CH3 IgG1 Fc | 443 | 454 |
| | SP-5 | SIRPαV2 IgV V33H-CL + CH1-CH2-CH3 IgG1 Fc | 444 | 454 |
| | SP-6 | SIRPαV2 IgV M72H-CL + CH1-CH2-CH3 IgG1 Fc | 445 | 454 |
| | SP-7 | SIRPαV2 IgV WT-CL + CH1-CH2-CH3 IgG1 Fc | 446 | 454 |
| | SP-8 | SIRPαV2 IgV WT-CL + CH1-CH2-CH3 IgG4 Fc | 446 | 455 |
| FV-4 | SP-9 | SIRPαV2 IgV K53H-K53H-CL + CH1-CH2-CH3 IgG1 Fc | 447 | 454 |
| | SP-10 | SIRPαV2 IgV R69H-R69H-CL + CH1-CH2-CH3 IgG1 Fc | 448 | 454 |
| | SP-11 | SIRPαV2 IgV K53H-R69H-CL + CH1-CH2-CH3 IgG1 Fc | 449 | 454 |
| | SP-12 | SIRPαV2 IgV R69H-K53H-CL + CH1-CH2-CH3 IgG1 Fc | 450 | 454 |
| | SP-13 | SIRPαV2 IgV V33H-V33H-CL + CH1-CH2-CH3 IgG1 Fc | 451 | 454 |
| | SP-14 | SIRPαV2 IgV M72H-M72H-CL + CH1-CH2-CH3 IgG1 | 452 | 454 |
| | SP-15 | SIRPαV2 IgV WT-WT-CL + CH1-CH2-CH3 IgG1 Fc | 453 | 454 |
| | SP-16 | SIRPαV2 IgV WT-WT-CL + CH1-CH2-CH3 IgG4 Fc | 453 | 455 |
| FV-5 | SP-17 | SIRPαV2 IgV K53H-CL + | 440 | 456 |
| | | SIRPαV2 IgV K53H-CH1-CH2-CH3 IgG1 Fc | | |
| | SP-18 | SIRPαV2 IgV R69H-CL + | 441 | 457 |
| | | SIRPαV2 IgV R69H-CH1-CH2-CH3 IgG1 Fc | | |
| | SP-19 | SIRPαV2 IgV K53H-CL + | 440 | 457 |
| | | SIRPαV2 IgV R69H-CH1-CH2-CH3 IgG1 Fc | | |
| | SP-20 | SIRPαV2 IgV R69H-CL + | 441 | 456 |
| | | SIRPαV2 IgV K53H-CH1-CH2-CH3 IgG1 Fc | | |
| | SP-21 | SIRPαV2 IgV Q52H-CL + | 442 | 457 |
| | | SIRPαV2 IgV R69H-CH1-CH2-CH3 IgG1 Fc | | |
| | SP-22 | SIRPαV2 IgV K68H-CL + | 443 | 457 |
| | | SIRPαV2 IgV R69H-CH1-CH2-CH3 IgG1 Fc | | |
| | SP-23 | SIRPαV2 IgV V33H-CL + | 444 | 458 |
| | | SIRPαV2 IgV V33H-CH1-CH2-CH3 IgG1 Fc | | |
| | SP-24 | SIRPαV2 IgV M72H-CL + | 445 | 459 |
| | | SIRPαV2 IgV M72H-CH1-CH2-CH3 IgG1 Fc | | |
| | SP-25 | SIRPαV2 IgV WT-CL + | 446 | 460 |
| | | SIRPαV2 IgV WT-CH1-CH2-CH3 IgG1 Fc | | |
| | SP-26 | SIRPαV2 IgV WT-CL + | 446 | 461 |
| | | SIRPαV2 IgV WT-CH1-CH2-CH3 IgG4 Fc | | |
| FV-3 | SIN-368 | SIRPαV2 IgV K53H-K53H-IgG1 Fc | 368 | — |
| FV-114 | SIN-370 | SIRPαV2 IgV R69H-IgG1 Fc-CD40L | 370 | — |

TABLE 20
Constructs of representative SIRP IgV antibody-fusion proteins of exemplary formats

| Exemplary | | | Chain 1 | Chain 2 | Chain 3 |
|---|---|---|---|---|---|
| format | Protein | | SEQ ID | SEQ ID | SEQ ID |
| variant | ID | Description of representative protein | NO: | NO: | NO: |
| FV-48 | MP-2 | aDectin-1 VH-CH1-IgG1 Fc silent + | 379 | 383 | — |
| | | SIRPαV2 IgV WT-aDectin-1 VL-CL | | | |
| | MP-3 | aPD-L1 VH-CH1-IgG1 Fc + SIRPαV2 IgV WT-aPD-L1 VL-CL | 384 | 394 | — |
| | MP-29 | aPD-L1 VH-CH1-IgG1 Fc + SIRPαV2 IgV K53H-aPD-L1 VL-CL | 386 | 395 | — |
| | MP-30 | aPD-L1 VH-CH1-IgG1 Fc + SIRPαV2 IgV R69H-aPD-L1 VL-CL | 386 | 396 | — |
| | MP-31 | aPD-L1 VH-CH1-IgG1 Fc + SIRPαV2 IgV Q52H-aPD-L1 VL-CL | 386 | 397 | — |
| | MP-32 | aPD-L1 VH-CH1-IgG1 Fc + SIRPαV2 IgV K68H-aPD-L1 VL-CL | 386 | 398 | — |
| | MP-33 | aPD-L1 VH-CH1-IgG1 Fc + SIRPαV2 IgV V33H-aPD-L1 VL-CL | 386 | 462 | — |
| | MP-34 | aPD-L1 VH-CH1-IgG1 Fc + SIRPαV2 IgV M72H-aPD-L1 VL-CL | 386 | 463 | — |
| | MP-35 | aPD-L1 VH-CH1-IgG1 Fc + SIRPαV2 IgV G97H-aPD-L1 VL-CL | 386 | 464 | — |
| | MP-36 | Isotype VH-CH1-IgG1 Fc + SIRPαV2 IgV K53H-Isotype VL-CL | 471 | 465 | — |
| | MP-37 | aCD70 VH-CH1-IgG1 Fc + SIRPαV2 IgV K53H-aCD70 VL-CL | 472 | 466 | — |
| | MP-38 | aEGFR VH-CH1-IgG1 Fc + SIRPαV2 IgV K53H-aEGFR VL-CL | 407 | 467 | — |
| FV-49 | MP-4 | SIRPαV2 IgV WT-aPD-L1 VH-CH1-IgG1 Fc + aPD-L1 VL-CL | 385 | 393 | — |
| | MP-39 | SIRPαV2 IgV K53H-aPD-L1 VH-CH1-IgG1 Fc + aPD-L1 VL-CL | 387 | 393 | — |
| | MP-40 | SIRPαV2 IgV R69H-aPD-L1 VH-CH1-IgG1 Fc + aPD-L1 VL-CL | 388 | 393 | — |
| | MP-41 | SIRPαV2 IgV Q52H-aPD-L1 VH-CH1-IgG1 Fc + aPD-L1 VL-CL | 389 | 393 | — |
| | MP-42 | SIRPαV2 IgV K68H-aPD-L1 VH-CH1-IgG1 Fc + aPD-L1 VL-CL | 390 | 393 | — |
| | MP-43 | SIRPαV2 IgV V33H-aPD-L1 VH-CH1-IgG1 Fc + aPD-L1 VL-CL | 473 | 393 | — |
| | MP-44 | SIRPαV2 IgV M72H-aPD-L1 VH-CH1-IgG1 Fc + aPD-L1 VL-CL | 474 | 393 | — |
| | MP-45 | SIRPαV2 IgV G97H-aPD-L1 VH-CH1-IgG1 Fc + aPD-L1 VL-CL | 475 | 393 | — |
| | MP-46 | SIRPαV2 IgV K53H-Isotype VH-CH1-IgG1 Fc + Isotype VL-CL | 476 | 468 | — |
| | MP-47 | SIRPαV2 IgV K53H-aCD70 VH-CH1-IgG1 Fc + aCD70 VL-CL | 477 | 469 | — |
| | MP-48 | SIRPαV2 IgV K53H-aEGFR VH-CH1-IgG1 Fc + aEGFR VL-CL | 478 | 470 | — |
| FV-54 | MP-5 | SIRPαV2 IgV WT-aPD-L1 VH-CH1-IgG1 Fc + | 385 | 394 | — |
| | | SIRPαV2 IgV WT-aPD-L1 VL-CL | | | |
| | MP-6 | SIRPαV2 IgV K53H-aPD-L1 VH-CH1-IgG1 Fc + | 387 | 395 | — |
| | | SIRPαV2 IgV K53H-aPD-L1 VL-CL | | | |
| | MP-16 | SIRPαV2 IgV K53H + N80A-aPD L1 VH-CH1-IgG1 Fc + | 391 | 399 | — |
| | | SIRPαV2 IgV K53H + N80A-aPD-L1 VL-CL | | | |
| | MP-7 | SIRPαV2 IgV R69H-aPD-L1 VH-CH1-IgG1 Fc + | 388 | 396 | — |
| | | SIRPαV2 IgV R69H-aPD-L1 VL-CL | | | |
| | MP-8 | SIRPαV2 IgV Q52H-aPD-L1 VH-CH1-IgG1 Fc + | 389 | 396 | — |
| | | SIRPαV2 IgV R69H-aPD-L1 VL-CL | | | |
| | MP-9 | SIRPαV2 IgV K68H-aPD-L1 VH-CH1-IgG1 Fc + | 390 | 396 | — |
| | | SIRPαV2 IgV R69H-aPD-L1 VL-CL | | | |
| | MP-10 | SIRPαV2 IgV Q52H-aPD-L1 VH-CH1-IgG1 Fc + | 389 | 397 | — |
| | | SIRPαV2 IgV Q52H-aPD-L1 VL-CL | | | |
| | MP-11 | SIRPαV2 IgV K68H-aPD-L1 VH-CH1-IgG1 Fc + | 390 | 398 | — |
| | | SIRPαV2 IgV K68H-aPD-L1 VL-CL | | | |
| | MP-49 | SIRPαV2 IgV K53H-aPD-L1 VH-CH1-IgG1 Fc + | 387 | 396 | — |
| | | SIRPαV2 IgV R69H-aPD-L1 VL-CL | | | |
| | MP-50 | SIRPαV2 IgV R69H-aPD-L1 VH-CH1-IgG1 Fc + | 388 | 395 | — |
| | | SIRPαV2 IgV K53H-aPD-L1 VL-CL | | | |
| | MP-51 | SIRPαV2 IgV V33H-aPD-L1 VH-CH1-IgG1 Fc + | 473 | 462 | — |
| | | SIRPαV2 IgV V33H-aPD-L1 VL-CL | | | |
| | MP-52 | SIRPαV2 IgV M72H-aPD-L1 VH-CH1-IgG1 Fc + | 474 | 463 | — |
| | | SIRPαV2 IgV M72H-aPD-L1 VL-CL | | | |
| | MP-53 | SIRPαV2 IgV G97H-aPD-L1 VH-CH1-IgG1 Fc + | 475 | 464 | — |
| | | SIRPαV2 IgV G97H-aPD-L1 VL-CL | | | |
| FV-50 | MP-12 | aPD-L1 VH-CH1-IgG1 Fc + SIRPαV2 IgV K53H-K53H-aPD-L1 VL-CL | 386 | 400 | — |
| | MP-13 | aPD-L1 VH-CH1-IgG1 Fc + SIRPαV2 IgV R69H-R69H-aPD-L1 VL-CL | 386 | 401 | — |
| | MP-14 | aPD-L1 VH-CH1-IgG1 Fc + SIRPαV2 IgV R69H-K68H-aPD-L1 VL-CL | 386 | 402 | — |
| | MP-15 | aPD-L1 VH-CH1-IgG1 Fc + SIRPαV2 IgV R69H-Q52H-aPD-L1 VL-CL | 386 | 403 | — |
| | MP-54 | aPD-L1 VH-CH1-IgG1 Fc + SIRPαV2 IgV K53H-R69H-aPD-L1 VL-CL | 386 | 479 | — |
| | MP-55 | aPD-L1 VH-CH1-IgG1 Fc + SIRPαV2 IgV V33H-V33H-aPD-L1 VL-CL | 386 | 480 | — |
| | MP-56 | aPD-L1 VH-CH1-IgG1 Fc + SIRPαV2 IgV M72H-M72H-aPD-L1 VL- | 386 | 481 | — |
| | MP-57 | aPD-L1 VH-CH1-IgG1 Fc + SIRPαV2 IgV WT-WT-aPD-L1 VL-CL | 386 | 482 | — |
| | MP-58 | aCD70 VH-CH1-IgG1 Fc + SIRPαV2 IgV K53H-K53H-aCD70 VL-CL | 472 | 483 | — |
| | MP-20 | aCD38 VH-CH1-IgG1 Fc + SIRPαV2 IgV K53H-K53H-aCD38 VL-CL | 405 | 406 | — |
| | MP-21 | aEGFR VH-CH1-IgG1 Fc + SIRPαV2 IgV K53H-K53H-aEGFR VL-CL | 407 | 408 | — |
| FV-51 | MP-59 | SIRPαV2 IgV K53H-K53H-aPD-L1 VH-CH1-IgG1 Fc + aPD-L1 VL-CL | 484 | 393 | — |
| | MP-60 | SIRPαV2 IgV R69H-R69H-aPD-L1 VH-CH1-IgG1 Fc + aPD-L1 VL-CL | 485 | 393 | — |
| | MP-61 | SIRPαV2 IgV K53H-R69H-aPD-L1 VH-CH1-IgG1 Fc + aPD-L1 VL-CL | 486 | 393 | — |
| | MP-62 | SIRPαV2 IgV M72H-M72H-aPD-L1 VH-CH1-IgG1 Fc + aPD-L1 VL- | 487 | 393 | — |
| | MP-63 | SIRPαV2 IgV WT-WT-aPD-L1 VH-CH1-IgG1 Fc + aPD-L1 VL-CL | 488 | 393 | — |
| FV-52 | MP-17 | aPD-L1 VH-CH1-IgG1 Fc + SIRPαV2 IgV K53H + N80A-K53H + N80A- | 386 | 404 | — |
| | | K53H + N80A-aPD-L1 VL-CL | | | |
| | MP-64 | aPD-L1 VH-CH1-IgG1 Fc + SIRPαV2 IgV K53H-K53H-R69H-aPD-L1 | 386 | 490 | — |
| | | VL-CL | | | |
| | MP-65 | aPD-L1 VH-CH1-IgG1 Fc + SIRPαV2 IgV R69H-K53H-K53H-aPD-L1 | 386 | 491 | — |
| | | VL-CL | | | |
| FV-53 | MP-66 | SIRPαV2 IgV K53H-R69H-R69H-aPD-L1 VH-CH1-IgG1 Fc + aPD-L1 | 489 | 393 | — |
| | | VL-CL | | | |
| FV-55 | MP-18 | SIRPαV2 IgV K53H-aPD-L1 VH-CH1-IgG1 Fc + (SIRPαV2 IgV | 387 | 400 | — |
| | | K53H)x2-aPD-L1 VL-CL | | | |
| | MP-19 | SIRPαV2 IgV R69H-aPD-L1 VH-CH1-IgG1 Fc + (SIRPαV2 IgV | 388 | 401 | — |
| | | R69H)x2-aPD-L1 VL-CL | | | |
| FV-67 | MP-24 | SIRPαV2 IgV WT-IgG1 Fc hole silent + aCD3 VH-CH1-IgG1 Fc | 415 | 411 | 414 |
| | | knob silent + aCD3 VL-CL | | | |
| | MP-25 | SIRPαV2 IgV K53H-IgG1 Fc hole silent + aCD3 VH-CH1-IgG1 Fc | 416 | 411 | 414 |
| | | knob silent + aCD3 VL-CL | | | |
| | MP-26 | SIRPαV2 IgV R69H-IgG1 Fc hole silent + aCD3 VH-CH1-IgG1 Fc | 417 | 411 | 414 |
| | | knob silent + aCD3 VL-CL | | | |
| | MP-67 | SIRPαV2 IgV Q52H-IgG1 Fc hole silent + aCD3 VH-CH1-IgG1 Fc | 492 | 411 | 414 |
| | | knob silent + aCD3 VL-CL | | | |
| | MP-68 | SIRPαV2 IgV K68H-IgG1 Fc hole silent + aCD3 VH-CH1-IgG1 Fc | 493 | 411 | 414 |
| | | knob silent + aCD3 VL-CL | | | |
| | MP-69 | SIRPαV2 IgV V33H-IgG1 Fc hole silent + aCD3 VH-CH1-IgG1 Fc | 494 | 411 | 414 |
| | | knob silent + aCD3 VL-CL | | | |
| | MP-70 | SIRPαV2 IgV M72H-IgG1 Fc hole silent + aCD3 VH-CH1-IgG1 Fc | 495 | 411 | 414 |
| | | knob silent + aCD3 VL-CL | | | |
| | MP-23 | SIRPαV2 IgV WT-IgG1 Fc hole silent + aCD205 VH-CH1-IgG1 Fc | 409 | 410 | 415 |
| | | knob silent + aCD205 VL-CL | | | |
| | MP-71 | SIRPαV2 IgV Q52H-IgG1 Fc hole + aPD-L1 VH-CH1-IgG1 Fc knob + | 492 | 498 | 393 |
| | | aPD-L1 VL-CL | | | |
| | MP-72 | SIRPαV2 IgV Q52H-IgG1 Fc hole + aCD70 VH-CH1-IgG1 Fc knob + | 492 | 499 | 469 |
| | | aCD70 VL-CL | | | |
| | MP-73 | SIRPαV2 IgV Q52H-IgG1 Fc hole + aEGFR VH-CH1-IgG1 Fc knob + | 492 | 500 | 470 |
| | | aEGFR VL-CL | | | |
| FV-64 | MP-74 | SIRPαV2-WT-aPD-L1 VH-CH1-IgG1 Fc hole + aPD-L1 VH-CH1- | 501 | 498 | 393 |
| | | IgG1 Fc knob + aPD-L1 VL-CL | | | |
| | MP-75 | SIRPαV2-Q52H-aPD-L1 VH-CH1-IgG1 Fc hole + aPD-L1 VH-CH1- | 502 | 498 | 393 |
| | | IgG1 Fc knob + aPD-L1 VL-CL | | | |
| | MP-76 | SIRPαV2-K68H-aPD-L1 VH-CH1-IgG1 Fc hole + aPD-L1 VH-CH1- | 503 | 498 | 393 |
| | | IgG1 Fc knob + aPD-L1 VL-CL | | | |
| | MP-77 | SIRPαV2-Q52H + K68H-aPD-L1 VH-CH1-IgG1 Fc hole + aPD-L1 VH- | 504 | 498 | 393 |
| | | CH1-IgG1 Fc knob + aPD-L1 VL-CL | | | |
| | MP-78 | SIRPαV2-M72H-aPD-L1 VH-CH1-IgG1 Fc hole + aPD-L1 VH-CH1- | 505 | 498 | 393 |
| | | IgG1 Fc knob + aPD-L1 VL-CL | | | |
| | MP-79 | SIRPαV2-Q52H-aCD70 VH-CH1-IgG1 Fc hole + aCD70 VH-CH1- | 506 | 499 | 469 |
| | | IgG1 Fc knob + aCD70 VL-CL | | | |
| | MP-80 | SIRPαV2-Q52H-aEGFR VH-CH1-IgG1 Fc hole + aEGFR VH-CH1- | 507 | 500 | 470 |
| | | IgG1 Fc knob + aEGFR VL-CL | | | |
| FV-72 | MP-27 | SIRPαV2 IgV K53H-IgG1 Fc hole silent + SIRPαV2 IgV K53H-aCD3 | 412 | 411 | 416 |
| | | VH-CH1-IgG1 Fc knob silent + aCD3 VL-CL | | | |
| | MP-28 | SIRPαV2 IgV R69H-IgG1 Fc hole silent + SIRPαV2 IgV R69H-aCD3 | 417 | 413 | 414 |
| | | VH-CH1-IgG1 Fc knob silent + aCD3 VL-CL | | | |
| | MP-81 | SIRPαV2 IgV K53H-IgG1 Fc hole silent + SIRPαV2 IgV R69H-aCD3 | 416 | 413 | 414 |
| | | VH-CH1-IgG1 Fc knob silent + aCD3 VL-CL | | | |
| | MP-82 | SIRPαV2 IgV WT-IgG1 Fc hole + SIRPαV2 IgV R69H + K68H-aPD-L1 | 415 | 508 | 393 |
| | | VH-CH1-IgG1 Fc knob + aPD-L1 VL-CL | | | |
| | MP-83 | SIRPαV2 IgV M72H-IgG1 Fc hole silent + SIRPαV2 IgV M72H- | 495 | 509 | 414 |
| | | aCD3 VH-CH1-IgG1 Fc knob silent + aCD3 VL-CL | | | |
| | MP-84 | SIRPαV2 IgV WT-IgG1 Fc hole silent + SIRPαV2 IgV WT-aCD3 VH- | 415 | 510 | 414 |
| | | CH1-IgG1 Fc knob silent + aCD3 VL-CL | | | |
| FV-136 | MP-22 | aPD-L1 VH-CH1-IgG1 Fc silent-TGFBRII + SIRPαV2 IgV WT-aPD- | 392 | 394 | — |
| | | L1 VL-CL | | | |
| | MP-85 | aPD-L1 VH-CH1-IgG4 Fc-TGFβRII + SIRPαV2 IgV WT-aPD-L1 VL- | 511 | 394 | — |
| | | CL | | | |
| | MP-86 | aPD-L1 VH-CH1-IgG1 Fc-TGFβRII + SIRPαV2 IgV K53H-aPD-L1 VL- | 512 | 395 | — |
| | | CL | | | |

TABLE 21
Constructs of representative SIRP IgV Fab-fusion proteins
and other fusion proteins of exemplary formats

| Exemplary | | | Chain 1 | Chain 2 |
|---|---|---|---|---|
| format | | | SEQ ID | SEQ ID |
| variant | Protein ID | Description of representative protein | NO: | NO: |
| FV-32a | MP-87 | SIRPαV2 IgV K53H-K53H-K53H-K53H- | 378 | 380 |
| | | aDectin-1 VH-CH1 + aDectin-1 VL-CL | | |
| FV-32c | MP-88 | aDectin-1 VH-CH1-SIRPαV2 IgV K53H-K53H- | 376 | 380 |
| | | K53H-K53H + aDectin-1 VL-CL | | |
| FV-34b | MP-89 | aDectin-1 VH-CH1-SIRPαV2 IgV K53H-K53H + | 375 | 382 |
| | | aDectin-1 VL-CL-SIRPαV2 IgV K53H-K53H | | |
| | MP-90 | aDectin-1 VH-CH1-SIRPαV2 IgV Q52H-K53H + | 374 | 381 |
| | | aDectin-1 VL-CL-SIRPαV2 IgV Q52H-K53H | | |
| FV-43b | MP-91 | aDectin-1 VH-CH1-aHER2 Trastuzumab | 373 | 377 |
| | | scFv + aDectin-1 VL-CL-SIRPαV2 IgV WT | | |
| | MP-92 | aDectin-1 VH-CH1-aHER2 Trastuzumab | 373 | 383 |
| | | scFv + aDectin-1 VL-CL-SIRPαV2 IgV Q52H | | |
| FV-44b | MP-93 | aDectin-1 VH-CH1-aHER2 Trastuzumab | 373 | 382 |
| | | scFv + aDectin-1 VL-CL-SIRPαV2 IgV K53H- | | |
| FV-47a | MP-1 | aDectin-1 VH-CH1-aHER2 Trastuzumab | 373 | 380 |
| | | scFv + aDectin-1 VL-CL | | |
| FV-20a | SIN-371 | SIRPαV2 IgV R69H-G-4-1BBL | 371 | — |
| FV-27a | SIN-372 | SIRPαV2 IgV R69H-aHSA VHH-4-1BBL | 372 | — |

TABLE 22
Production of representative protein if standalone SIRP IgV multimer polypeptide

| Protein name | SIN-418 | SIN-419 | SIN-422 | SIN-424 | SIN-425 | SIN-429 |
|---|---|---|---|---|---|---|
| Format variant | FV-19a | FV-19a | FV-19a | FV-19a | FV-19a | FV-19a |
| SIRP IgV | 418 | 419 | 422 | 424 | 425 | 429 |
| multimer | | | | | | |
| polypeptide | | | | | | |
| SEQ ID NO: | | | | | | |
| Yield (mg/L) | 168.7 | 160 | 190 | 138 | 190 | 253.3 |
| Molecular | 30 KD | 30 KD | 30 KD | 30 KD | 30 KD | 30 KD |
| weight | | | | | | |
| (through SDS- | | | | | | |
| SDS-PAGE | >95% | >95% | >95% | >95% | >95% | >95% |
| purity | | | | | | |
| SEC-HPLC | 100.0% | 85.2% | 97.1% | 74.0% | 96.9% | 100.0% |
| purity | | | | | | |

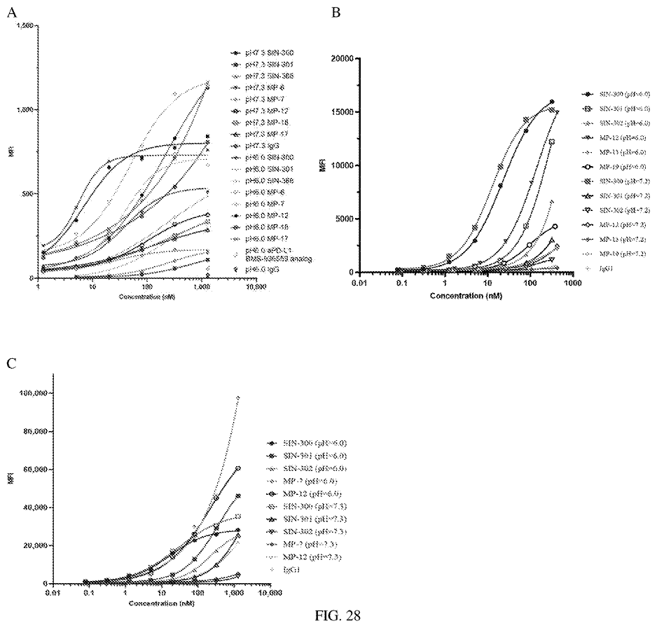

Meanwhile, the FACS binding assay results on Raji cells and SK-OV-3 cells in FIG. 28A-C generally show similar pattern as the ELISA results in FIG. 27A-D, but with several surprising discrepancy: 1) the results in FIG. 28A show that the texavalent protein MP-18 and MP-17 surprisingly exhibited lower binding to Raji cells than the tetravalent counterparts MP-6 and MP-12 at both acidic and physiological pH (FIG. 28A), despite showing oppositely stronger binding in ELISA assay (FIG. 27B). These results however further demonstrate tetravelency of the pH-differential variant SIRP IgV monomers unexpectedly as the more favorable valency than the higher hexavalency; 2) the results in FIG. 28B show that the tetravalent SIRP IgV fusion protein MP-13 and the hexavalent SIRP IgV fusion protein MP-19 with R69H mutation surprisingly showed actually weaker binding to Raji cells than the bivalent counterpart SIN-302 with R69H mutation (FIG. 28B), despite showing oppositely higher binding in ELISA assay (FIG. 27A). However, both MP-13 and MP-19 continued to exhibit higher binding at an acidic pH than at physiological pH (FIG. 28B). Also, the other tetravalent SIRP IgV fusion protein MP-7 with R69H mutation exhibited higher binding at an acidic pH (FIG. 28A, 28C), consistent with the ELISA result. The unexpected discrepancy in the FACS results may be resulted from interplay of specific spatial distribution pattern of cell surface CD47 protein e.g. on Raji cells with the spatial format of the multivalent SIRP IgV monomers in MP-13 and MP-19 and the binding affinity of the monovalent SIRP IgV monomer component.

### Example 11. Binding Activities of Fusion Proteins Comprising CD47-Binding SIRP IgV Domains at Different Valency and Formats to Human Primary Platelets and T Cells Measured by FACS

The CD47-binding fusion proteins of various valency and formats, as tested in Example 10 above, were further tested for binding to human primary platelets and T cells at physiological pH through FACS binding assay, as similarly done in Example 4 and 5 above.

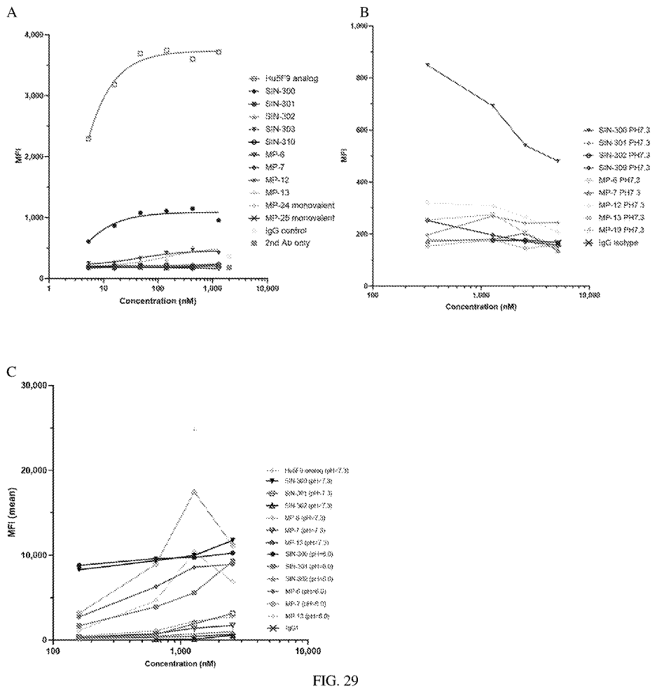

The human platelet FACS results in FIG. 29A and B show that similar to the bivalent counterparts SIN-302 and SIN-309 with R69H mutation, the tetravalent SIRP IgV fusion protein MP-7 and MP-13 as well as the hexavalent SIRP IgV fusion protein MP-19 with R69H mutation maintained negligible binding to human platelets even at higher concentrations up to 5,125 nM or 410 μg/ml tested, which reached around the reported Week 1 peak serum concentration of the highest 18.0 mg/kg IV dose of TTI-622 tested in clinical trials (Trillium Therapeutics, Inc. R&D Day presentation, Apr. 28, 2021). Also similar to the bivalent counterparts SIN-301 and SIN-310 with K53H mutation, the tetravalent SIRP IgV fusion protein MP-6, and MP-12 with K53H mutation, as well as the monovalent SIRP IgV fusion protein MP-25 with K53H mutation, exhibited negligible or weak binding to human platelets at physiological pH 7.3 (FIG. 29A-B). Meanwhile, the bivalent SIRP IgV fusion protein SIN-303 with Q52H mutation exhibited platelet binding similar to that of the benchmark protein MP-24 with monovalent wild type SIRPαV2 IgV domain that is much weaker than that of the benchmark protein SIN-300 (analog to clinically tested TTI-621) with bivalent wild type SIRPαV2 IgV domain (FIG. 29A). These results showed that the SIRP IgV fusion proteins with different valency and formats of SIRP IgV monomers comprising the mutation of K53H or R69H favorably exhibited negligible or low binding to human platelets even at high clinical dose levels, which is desirable to avoid or reduce thrombocytopenia in human patients. Similarly, the human T cell FACS results in FIG. 29C show that similar to the bivalent counterparts SIN-301 and SIN-302, the tetravalent SIRP IgV fusion proteins MP-6, MP-7 and MP-13 with K53H or R69H mutation exhibited negligible or weak binding at physiological pH 7.3 to human T cells even at high concentrations up to 2,560 nM tested. However at an acidic pH 6.0, MP-6, MP-7 and MP-13 exhibit significant binding to human T cells at or above 640 nM (FIG. 29C). Notably, the lack of significant avidity effect of tetravalency versus bivalency of the SIRP IgV monomers with K53H or R69H mutation at physiological pH in the FACS binding assay of human platelets and T cells indicates that a certain level of cell surface CD47 expression, e.g. higher than the CD47 expression level on human platelets and T cells, is needed to enable significant binding avidity effect from increasing valency of the SIRP IgV domains with K53H or R69H mutation that exhibit low monovalent binding affinity at physiological pH. This is favorable for proteins comprising multivalent SIRP IgV monomers with K53H or R69H mutation to avoid or reduce undesirable increase in CD47 binding to normal tissues at physiological pH, while allow desirable increasing avidity binding at both acidic and physiological pH to disease cells such as tumor cells that express increased level of surface CD47.

While the present embodiments and examples have been particularly shown and described with reference to example embodiments herein, it will be understood by those of ordinary skill in the art that various changes in form and details may be made therein without departing from the spirit and scope of the present embodiments and examples as defined by the following claims. Those skilled in the art will recognize, or be able to ascertain using no more than routine experimentation, numerous equivalents to the specific procedures described herein. Such equivalents are considered to be within the scope of the present invention and are covered by the following claims. The contents of all non-patent literature publications, patents, and patent applications cited throughout this application are hereby incorporated by reference. The appropriate components, processes, and methods of those patents, applications and other documents may be selected for the present invention and embodiments thereof.

### REFERENCE

Amouzegar, A., M. Chelvanambi, J. N. Filderman, W. J. Storkus and J. J. Luke (2021). “STING Agonists as Cancer Therapeutics.” Cancers (Basel) 13 (11).
Anwar, M. A., M. Shah, J. Kim and S. Choi (2019). “Recent clinical trends in Toll-like receptor targeting therapeutics.” Med Res Rev 39 (3): 1053-1090. Baeuerle, P. A., J. Ding, E. Patel, N. Thorausch, H. Horton, J. Gierut, I. Scarfo, R. Choudhary, O. Kiner, J. Krishnamurthy, B. Le, A. Morath, G. C. Baldeviano, J. Quinn, P. Tavares, Q. Wei, S. Weiler, M. V. Maus, D. Getts, W. W. Schamel and R. Hofmeister (2019). “Synthetic TRUC receptors engaging the complete T cell receptor for potent anti-tumor response.” Nat Commun 10 (1): 2087.
Barclay, A. N. and M. H. Brown (2006). “The SIRP family of receptors and immune regulation.” Nat Rev Immunol 6 (6): 457-464.
Biederstadt, A. and K. Rezvani (2021). “Engineering the next generation of CAR-NK immunotherapies.” Int J Hematol 114 (5): 554-571.
Chao, M. P., S. Jaiswal, R. Weissman-Tsukamoto, A. A. Alizadeh, A. J. Gentles, J. Volkmer, K. Weiskopf, S. B. Willingham, T. Raveh, C. Y. Park, R. Majeti and I. L. Weissman (2010). “Calreticulin is the dominant pro-phagocytic signal on multiple human cancers and is counterbalanced by CD47.” Sci Transl Med 2 (63): 63ra94.
Corneillie, T. M., P. A. Whetstone, A. J. Fisher and C. F. Meares (2003). “A rare earth-DOTA-binding antibody: probe properties and binding affinity across the lanthanide series.” J Am Chem Soc 125 (12): 3436-3437.
Dubowchik, G. M. and M. A. Walker (1999). “Receptor-mediated and enzyme-dependent targeting of cytotoxic anticancer drugs.” Pharmacol Ther 83 (2): 67-123.
Erra Diaz, F., E. Dantas and J. Geffner (2018). “Unravelling the Interplay between Extracellular Acidosis and Immune Cells.” Mediators Inflamm 2018:1218297.
Feng, M., K. D. Marjon, F. Zhu, R. Weissman-Tsukamoto, A. Levett, K. Sullivan, K. S. Kao, M. Markovic, P. A. Bump, H. M. Jackson, T. S. Choi, J. Chen, A. M. Banuelos, J. Liu, P. Gip, L. Cheng, D. Wang and I. L. Weissman (2018). “Programmed cell removal by calreticulin in tissue homeostasis and cancer.” Nat Commun 9 (1): 3194.
Frega, G., Q. Wu, J. Le Naour, E. Vacchelli, L. Galluzzi, G. Kroemer and O. Kepp (2020). “Trial Watch: experimental TLR7/TLR8 agonists for oncological indications.” Oncoimmunology 9 (1): 1796002.
Fujiwara, K., A. Tsunei, H. Kusabuka, E. Ogaki, M. Tachibana and N. Okada (2020). “Hinge and Transmembrane Domains of Chimeric Antigen Receptor Regulate Receptor Expression and Signaling Threshold.” Cells 9 (5). Gao, T. A. and Y. Y. Chen (2022). “Engineering Next-Generation CAR-T Cells: Overcoming Tumor Hypoxia and Metabolism.” Annu Rev Chem Biomol Eng 13:193-216.
Gauzy-Lazo, L., I. Sassoon and M. P. Brun (2020). “Advances in Antibody-Drug Conjugate Design: Current Clinical Landscape and Future Innovations.” SLAS Discov 25 (8): 843-868.
Gheibibayat, S. M., R. Cabezas, N. G. Nikiforov, T. Jamialahmadi, T. P. Johnston and A. Sahebkar (2021). “CD47 in the Brain and Neurodegeneration: An Update on the Role in Neuroinflammatory Pathways.” Molecules 26 (13).
Golubovskaya, V., R. Berahovich, H. Zhou, S. Xu, H. Harto, L. Li, C. C. Chao, M. M. Mao and L. Wu (2017). “CD47-CAR-T Cells Effectively Kill Target Cancer Cells and Block Pancreatic Tumor Growth.” Cancers (Basel) 9 (10).
Guedan, S., H. Calderon, A. D. Posey, Jr. and M. V. Maus (2019). “Engineering and Design of Chimeric Antigen Receptors.” Mol Ther Methods Clin Dev 12:145-156.
Hatherley, D., S. C. Graham, J. Turner, K. Harlos, D. I. Stuart and A. N. Barclay (2008). “Paired receptor specificity explained by structures of signal regulatory proteins alone and complexed with CD47.” Mol Cell 31 (2): 266-277.
Hatherley, D., S. M. Lea, S. Johnson and A. N. Barclay (2014). “Polymorphisms in the human inhibitory signal-regulatory protein alpha do not affect binding to its ligand CD47.” J Biol Chem 289 (14): 10024-10028.
Helsen, C. W., J. A. Hammill, V. W. C. Lau, K. A. Mwawasi, A. Afsahi, K. Bezverbnaya, L. Newhook, D. L.
Hayes, C. Aarts, B. Bojovic, G. F. Denisova, J. M. Kwiecien, I. Brain, H. Derocher, K. Milne, B. H. Nelson and J. L. Bramson (2018). “The chimeric TAC receptor co-opts the T cell receptor yielding robust anti-tumor activity without toxicity.” Nat Commun 9 (1): 3049.
Hong, M., J. D. Clubb and Y. Y. Chen (2020). “Engineering CAR-T Cells for Next-Generation Cancer Therapy.” Cancer Cell 38 (4): 473-488.
Hossian, A., C. S. Hackett, R. J. Brentjens and S. Rafiq (2022). “Multipurposing CARs: Same engine, different vehicles.” Mol Ther 30 (4): 1381-1395.
Iurescia, S., D. Fioretti and M. Rinaldi (2020). “The Innate Immune Signalling Pathways: Turning RIG-1 Sensor Activation Against Cancer.” Cancers (Basel) 12 (11).
Jayaraman, J., M. P. Mellody, A. J. Hou, R. P. Desai, A. W. Fung, A. H. T. Pham, Y. Y. Chen and W. Zhao (2020). “CAR-T design: Elements and their synergistic function.” EBioMedicine 58:102931.
Jones, K. M., E. A. Randtke, E. S. Yoshimaru, C. M. Howison, P. Chalasani, R. R. Klein, S. K. Chambers, P. H. Kuo and M. D. Pagel (2017). “Clinical Translation of Tumor Acidosis Measurements with AcidoCEST MRI.” Mol Imaging Biol 19 (4): 617-625.
Jonnalagadda, M., A. Mardiros, R. Urak, X. Wang, L. J. Hoffman, A. Bernanke, W. C. Chang, W. Bretzlaff, R. Starr, S. Priceman, J. R. Ostberg, S. J. Forman and C. E. Brown (2015). “Chimeric antigen receptors with mutated IgG4 Fc spacer avoid fc receptor binding and improve T cell persistence and antitumor efficacy.” Mol Ther 23 (4): 757-768.
Khongorzul, P., C. J. Ling. F. U. Khan, A. U. Ihsan and J. Zhang (2020). “Antibody-Drug Conjugates: A Comprehensive Review.” Mol Cancer Res 18 (1): 3-19.
Kim, J. S., S. Y. Jun and Y. S. Kim (2020). “Critical Issues in the Development of Immunotoxins for Anticancer Therapy.” J Pharm Sci 109 (1): 104-115.
Kojima, Y., J. P. Volkmer, K. McKenna, M. Civelek, A. J. Lusis, C. L. Miller, D. Direnzo, V. Nanda, J. Ye, A. J. Connolly, E. E. Schadt, T. Quertermous, P. Betancur, L. Maegdefessel, L. P. Matic, U. Hedin, I. L. Weissman and N. J. Leeper (2016). “CD47-blocking antibodies restore phagocytosis and prevent atherosclerosis.” Nature 536 (7614): 86-90
Kui Chen, Y. W., Haojun Liang, Huan Huang, Yuelan Liang, Jiaxin Zhang, Ya-nan Chang, Juan Lia, Min Fang, Gengmei Xing (2021). “Fullerenols boosting the therapeutic effect of anti-CD47 antibody to trigger robust anti-tumor immunity by inducing calreticulin exposure.” Nano Today 37.
Lee, W. Y., D. A. Weber, O. Laur, E. A. Severson, I. McCall, R. P. Jen, A. C. Chin, T. Wu, K. M. Gernert and C. A. Parkos (2007). “Novel structural determinants on SIRP alpha that mediate binding to CD47.” J Immunol 179 (11): 7741-7750.
Lerbs, T., L. Cui, M. E. King, T. Chai, C. Muscat, L. Chung, R. Brown, K. Rieger, T. Shibata and G. Wernig (2020). “CD47 prevents the elimination of diseased fibroblasts in scleroderma.” JCI Insight 5 (16).
Li, D. and M. Wu (2021). “Pattern recognition receptors in health and diseases.” Signal Transduct Target Ther 6 (1): 291.
Linl, C., H. Pei2, X. Yuanl, J. Xul, M. Zoul, Z. Chenl, A. Fossierl, M. Liul, S. Gool, G. Yingl, C. Tangl, L. Leil, W. Zhangl, Z. Wangl, Y. Hul, Z. Wangl, Y. Zhaol, Q. Hul, H. Jil and R. Chenl (2022). “A novel single TCR gamma chain fused with antibody-based binding domain facilitates T cell cytotoxicity for potent anti-tumor response without forming TCR-CD3 complex.” Research Square.
Liu, X., L. Liu, Z. Ren, K. Yang, H. Xu, Y. Luan, K. Fu, J. Guo, H. Peng, M. Zhu and Y. X. Fu (2018). “Dual Targeting of Innate and Adaptive Checkpoints on Tumor Cells Limits Immune Evasion.” Cell Rep 24 (8): 2101-2111.
Liu, Y., G. Liu, J. Wang, Z. Y. Zheng, L. Jia, W. Rui, D. Huang, Z. X. Zhou, L. Zhou, X. Wu, S. Lin, X. Zhao and X. Lin (2021). “Chimeric STAR receptors using TCR machinery mediate robust responses against solid tumors.” Sci Transl Med 13 (586).
Liu, Y., Q. Tong, Y. Zhou, H. W. Lee, J. J. Yang, H. J. Buhring, Y. T. Chen, B. Ha, C. X. Chen, Y. Yang and K. Zen (2007). “Functional elements on SIRPαlpha IgV domain mediate cell surface binding to CD47.” J Mol Biol 365 (3): 680-693.
Maczynska, J., C. Da Pieve, T. A. Burley, F. Raes, A. Shah, J. Saczko, K. J. Harrington and G. Kramer-Marek (2020). “Immunomodulatory activity of IR700-labelled affibody targeting HER2.” Cell Death Dis 11 (10): 886.
Morais, M. and M. T. Ma (2018). “Site-specific chelator-antibody conjugation for PET and SPECT imaging with radiometals.” Drug Discov Today Technol 30:91-104.
Naghavi, M., R. John, S. Naguib, M. S. Siadaty, R. Grasu, K. C. Kurian, W. B. van Winkle, B. Soller, S. Litovsky, M. Madjid, J. T. Willerson and W. Casscells (2002). “pH Heterogeneity of human and rabbit atherosclerotic plaques; a new insight into detection of vulnerable plaque.” Atherosclerosis 164 (1): 27-35.
Neuberger, M. S., G. T. Williams and R. O. Fox (1984). “Recombinant antibodies possessing novel effector functions.” Nature 312 (5995): 604-608
Obeid, M., A. Tesniere, F. Ghiringhelli, G. M. Fimia, L. Apetoh, J. L. Perfettini, M. Castedo, G. Mignot, T. Panaretakis, N. Casares, D. Metivier, N. Larochette, P. van Endert, F. Ciccosanti, M. Piacentini, L. Zitvogel and G. Kroemer (2007). “Calreticulin exposure dictates the immunogenicity of cancer cell death.” Nat Med 13 (1): 54-61.
Obeid, M., A. Tesniere, T. Panaretakis, R. Tufi, N. Joza, P. van Endert, F. Ghiringhelli, L. Apetoh, N. Chaput, C. Flament, E. Ullrich, S. de Botton, L. Zitvogel and G. Kroemer (2007). “Ecto-calreticulin in immunogenic chemotherapy.” Immunol Rev 220:22-34.
Petrov, J. C., M. Wada, K. G. Pinz, L. E. Yan, K. H. Chen, X. Shuai, H. Liu, X. Chen, L. H. Leung, H. Salman, N. Hagag, F. Liu, X. Jiang and Y. Ma (2018). “Compound CAR T-cells as a double-pronged approach for treating acute myeloid leukemia.” Leukemia 32 (6): 317-1326.
Puro, R. J., M. N. Bouchlaka, R. R. Hiebsch, B. J. Capoccia, M. J. Donio, P. T. Manning, W. A. Frazier, R. W. Karr and D. S. Pereira (2020). “Development of AO-176, a Next-Generation Humanized Anti-CD47 Antibody with Novel Anticancer Properties and Negligible Red Blood Cell Binding” Mol Cancer Ther 19 (3): 835-846. Qin, V. M., C. D'Souza, P. J. Neeson and J. J. Zbu (2021). “Chimeric Antigen Receptor beyond CAR-T Cells.” Cancers (Basel) 13 (3).
Schmidt, D., S. Ebrahimabadi, K. R. S. Gomes, G. de Moura Aguiar, M. Cariati Tirapelle, R. Nacasaki Silvestre, J. T. C. de Azevedo, D. Tadeu Covas and V. Picanco-Castro (2022). “Engineering CAR-NK cells: how to tune innate killer cells for cancer immunotherapy.” Immunother Adv 2 (1): Itac003.
Schneider, D., Y. Xiong, D. Wu, V. Nö11e, S. Schmitz, W. Haso, A. Kaiser, B. Dropulic and R. J. Orentas (2017). “A tandem CD19/CD20 CAR lentiviral vector drives on-target and off-target antigen modulation in leukemia cell lines.” J Immunother Cancer 5:42.
Sharma, S. K. and K. D. Bagshawe (2017). “Translating antibody directed enzyme prodrug therapy (ADEPT) and prospects for combination.” Expert Opin Biol Ther 17 (1): 1-13.
Sneddon, D. and B. Cornelissen (2021). “Emerging chelators for nuclear imaging.” Curr Opin Chem Biol 63:152-162.
Sumerdon, G. A., P. E. Rogers, C. M. Lombardo, K. E. Schnobrich, S. L. Melvin, E. D. Hobart, Tribby, II, S. D.
Stroupe and D. K. Johnson (1990). “An optimized antibody-chelator conjugate for imaging of carcinoembryonic antigen with indium-111.” Int J Rad Appl Instrum B 17 (2): 247-254.
Thakral, P., S. Singla, M. P. Yadav, A. Vasisht, A. Sharma, S. K. Gupta, C. S. Bal, Snehlata and A. Malhotra (2014). “An approach for conjugation of (177) Lu-DOTA-SCN-Rituximab (BioSim) & its evaluation for radioimmunotherapy of relapsed & refractory β-cell non Hodgkins lymphoma patients.” Indian J Med Res 139 (4): 544-554.
Thomas, S., K. Straathof, N. Himoudi, J. Anderson and M. Pule (2016). “An Optimized GD2-Targeting Retroviral Cassette for More Potent and Safer Cellular Therapy of Neuroblastoma and Other Cancers.” PLOS One 11 (3): e0152196.
Thorpe, P. E., P. M. Wallace, P. P. Knowles, M. G. Relf, A. N. Brown, G. J. Watson, R. E. Knyba, E. J. Wawrzynczak and D. C. Blakey (1987). “New coupling agents for the synthesis of immunotoxins containing a hindered disulfide bond with improved stability in vivo.” Cancer Res 47 (22): 5924-5931.
Tsuchikama, K. and Z. An (2018). “Antibody-drug conjugates: recent advances in conjugation and linker chemistries.” Protein Cell 9 (1): 33-46.
Vitetta, E. S., R. J. Fulton, R. D. May, M. Till and J. W. Uhr (1987). “Redesigning nature's poisons to create anti-tumor reagents.” Science 238 (4830): 1098-1104.
Vizovisek, M., D. Ristanovic, S. Menghini, M. G. Christiansen and S. Schuerle (2021). “The Tumor Proteolytic Landscape: A Challenging Frontier in Cancer Diagnosis and Therapy.” Int J Mol Sci 22 (5).
Voets, E., M. Parade, D. Lutje Hulsik, S. Spijkers, W. Janssen, J. Rens, I. Reinieren-Beeren, G. van den Tillaart, S. van Duijnhoven, L. Driessen, M. Habraken, P. van Zandvoort, J. Kreijtz, P. Vink, A. van Elsas and H. van Eenennaam (2019). “Functional characterization of the selective pan-allele anti-SIRPαlpha antibody ADU-1805 that blocks the SIRPαlpha-CD47 innate immune checkpoint.” J Immunother Cancer 7 (1): 340.
Walsh, S. J., J. D. Bargh, F. M. Dannheim, A. R. Hanby, H. Seki, A. J. Counsell, X. Ou, E. Fowler, N. Ashman, Y. Takada, A. Isidro-Llobet, J. S. Parker, J. S. Carroll and D. R. Spring (2021). “Site-selective modification strategies in antibody-drug conjugates.” Chem Soc Rev 50 (2): 1305-1353.
Wang, H., Y. Sun, X. Zhou, C. Chen, L. Jiao, W. Li, S. Gou, Y. Li, J. Du, G. Chen, W. Zhai, Y. Wu, Y. Qi and Y. Gao (2020). “CD47/SIRPαlpha blocking peptide identification and synergistic effect with irradiation for cancer immunotherapy.” J Immunother Cancer 8 (2).
Wernig, G., S. Y. Chen, L. Cui, C. Van Neste, J. M. Tsai, N. Kambham, H. Vogel, Y. Natkunam, D. G. Gilliland, G. Nolan and I. L. Weissman (2017). “Unifying mechanism for different fibrotic diseases.” Proc Natl Acad Sci USA 114 (18): 4757-4762.
Winston, S. E., S. A. Fuller, M. J. Evelegh and J. G. Hurrell (2001). “Conjugation of enzymes to antibodies.” Curr Protoc Mol Biol Chapter 11: Unit11 11.
Wu, Y, Z. Huang, R. Harrison, L. Liu, L. Zhu, Y. Situ and Y. Wang (2022). “Engineering CAR T cells for enhanced efficacy and safety.” APL Bioeng 6 (1): 011502.
Xu, Y., Z. Yang, L. H. Horan, P. Zhang, L. Liu, B. Zimdahl, S. Green, J. Lu, J. F. Morales, D. M. Barrett, S. A. Grupp, V. W. Chan, H. Liu and C. Liu (2018). “A novel antibody-TCR (AbTCR) platform combines Fab-based antigen recognition with gamma/delta-TCR signaling to facilitate T-cell cytotoxicity with low cytokine release.” Cell Discov 4:62.
Yan, H. and W. Chen (2021). “The Promise and Challenges of Cyclic Dinucleotides as Molecular Adjuvants for Vaccine Development.” Vaccines (Basel) 9 (8).
Yan, L. E., H. Zhang, M. Wada, L. Fang, J. Feng, W. Zhang, Q. Chen, Y. Cao, K. G. Pinz, K. H. Chen, J. C. Petrov, X. Chen, L. H. Leung, X. X. Fan, L. Senzel, X. Jiang, Y. Ma and W. Tse (2020). “Targeting Two Antigens Associated with β-ALL with CD19-CD123 Compound Car T Cell Therapy.” Stem Cell Rev Rep 16 (2): 385-396.
Yao, H., F. Jiang, A. Lu and G. Zhang (2016). “Methods to Design and Synthesize Antibody-Drug Conjugates (ADCs).” Int J Mol Sci 17 (2).
Yu, X. Y., W. Y. Qiu, F. Long, X. P. Yang, C. Zhang, L. Xu, H. Y. Chang, P. Du, X. J. Hou, Y. Z. Yu, D. D. Zeng, S. Wang and Z. W. Sun (2018). “A novel fully human anti-CD47 antibody as a potential therapy for human neoplasms with good safety.” Biochimie 151:54-66.
Yurkovetskiy, A. V., N. D. Bodyak, M. Yin, J. D. Thomas, S. M. Clardy, P. R. Conlon, C. A. Stevenson, A. Uttard, L. Qin, D. R. Gumerov, E. Ter-Ovanesyan, C. Bu, A. J. Johnson, V. R. Gurijala, D. McGillicuddy, M. J. DeVit, L. L. Poling, M. Protopopova, L. Xu, Q. Zhang, P. U. Park, D. A. Bergstrom and T. B. Lowinger (2021). “Dolaflexin: A Novel Antibody-Drug Conjugate Platform Featuring High Drug Loading and a Controlled Bystander Effect.” Mol Cancer Ther 20 (5): 885-895.
Zah, E., M. Y. Lin, A. Silva-Benedict, M. C. Jensen and Y. Y. Chen (2016). “T Cells Expressing CD19/CD20 Bispecific Chimeric Antigen Receptors Prevent Antigen Escape by Malignant B Cells.” Cancer Immunol Res 4 (6): 498-508.
Zahavi, D. and L. Weiner (2020). “Monoclonal Antibodies in Cancer Therapy.” Antibodies (Basel) 9 (3).
Zhang, W., Q. Huang, W. Xiao, Y. Zhao, J. Pi, H. Xu, H. Zhao, J. Xu, C. E. Evans and H. Jin (2020). “Advances in Anti-Tumor Treatments Targeting the CD47/SIRPαlpha Axis.” Front Immunol 11:18.
Zhao, Q., Y. Jiang, S. Xiang, P. J. Kaboli, J. Shen, Y. Zhao, X. Wu, F. Du, M. Li, C. H. Cho, J. Li, Q. Wen, T. Liu, T. Yi and Z. Xiao (2021). “Engineered TCR-T Cell Immunotherapy in Anticancer Precision Medicine: Pros and Cons.” Front Immunol 12:658753.

1. A variant of a CD47-binding IgV extracellular domain of SIRpα, SIRPβ or SIRPγ comprising a mutation, wherein the variant exhibits higher binding affinity to CD47 at an acidic pH than at physiological pH, wherein the acidic pH is less than 7.0 and the physiological pH is 7.2 to 7.5, and wherein the mutation comprises a substitution selected from the group consisting of: K53H, K53H+K68H, K53H+Q52H, K53H+K68H+Q52H, K53H+I31E, K53H+I31D, K53H+L31E, K53H+L31D, K53H+H101D, K53H+N101D, K53H+L37Q, K53H+N101D+L37Q, K53H+N101D+L31E, K53H+N101D+L31D, K53H+L37Q+L31E, K53H+L37Q+L31D, K53H+N101D+L37Q+L31E, and K53H+N101D+L37Q+L31D.

2. (canceled)

3. The variant of claim 1, wherein the variant comprises a variant of the IgV extracellular domain of SIRPγ wherein the variant comprising a mutation of K53H, K53H+K68H, K53H+Q52H, K53H+K68H+Q52H, K53H+I31E, K53H+I31D, K53H+L31E, K53H+L31D, K53H+H101D, K53H+N101D, K53H+L37Q, K53+N101D+L37Q, K53H+N101D+L31E, K53H+N101D+L31D, K53H+L37Q+L31E, K53H+L37Q+L31D, K53H+N101D+L37Q+L31E, and K53H+N101D+L37Q+L31D.

4.-16. (canceled)

17. The variant of claim 1, wherein the variant further comprises an additional mutation, wherein the additional mutation comprises one or more substitution selected from the group consisting of E3H/G3H, E3D/G3D, L4H, L4E, L4D, L4V, L4I, V6H/M6H, V6I/M6I, V6L/M6L, V6E, V6D, D10H/E10H, K11H, K11E, K11D, V15H, V15E, V15D, E19H/K19H, E19D/K19D, A21H, A21V, A21E, A21D, V27H/A27H, V27I/A27I, V27L/A27L, V27Q/A27Q, V27E, V27D, S29H, S29E, S29D, L30H, L30E, L30D, I31Q/L31Q, I31N/L31N, I31Y/L31Y, I31W/L31W, I31F/L31F, I31V/L31V, I31R/L31R, I31K/L31K, I31T/L31T, I31S/L31S, I31L, P32H, P32E, P32D, V33H, V33I, V33E, V33D, G34H, G34E, G34D, P35H, P35G, P35N, P35E, P35D, I36H/V36H, I36E/V36E, I36D/V36D, Q37H/L37H, Q37E/L37E, Q37D/L37D, Q37V/L37V, Q37W/L37W, Q37A/L37A, W38H, W38E, W38D, F39H, F39E, F39D, R40H, R40E, R40D, R46H, R46E, R46D, E47H, E47D, E47V, E47L, E47Y, E47Q, L48H, L48E, L48D, I49H, I49E, I49D, Y50H, Y50E, Y50D, N51H, N51D, N51E, Q52E, Q52D, Q52G, K53R, K53E, K53D, E54H, E54D, E54Q, E54N, E54P, E54S, E54G, G55H, G55E, G55D, G55R, G55K, H56P, H56Y, H56R, H56D, H56E, F57H, F57E, F57D, P58H, P58E, P58D, P58R, P58K, R59H, R59E, R59D, V60H, V60E, V60D, T61H, T61E, T61D, T62H, T62E, T62D, V63I, V63A, V63H, V63E, V63D, S64H, S64E, S64D, E65H/D65H, E65R/D65R, S66H/L66H, S66T/L66T, S66G/L66G, S66E/L66E, S66D/L66D, S66Q/L66Q, S66W/L66W, S66A/L66A, T67H, T67E, T67D, T67W, K68R, K68A, K68E, K68D, K68I, K68T, R69E, R69D, E70H/N70H, E70D/N70D, N71H, N71D, N71E, M72H/L72H, M72N/L72N, M72R/L72R, M72E, M72D, M72I/L72I, M72W/L72W, F74H, F74E, F74D, S75H/P75H, S75E/P75E, SP7D/P75D, 176H, I76E, I76D, S77H/R77H, S77K/R77K, S77E/R77E, S77D/R77D, S77N/R77N, S79H/G79H, S79E/G79E, S79D/G79D, N80A, N80S, N80Q, N80H/S80H, D85H, D85E, Y89H, Y89E, Y89D, V92H, V92E, V92D, V92I, V92N, V92S, K93H, K93E, K93D, F94H, F94E, F94D, F94L, F94V, R95H, R95E, R95D, K96H, K96R, K96E, K96D, G97H, G97E, G97D, S98H, S98E, S98D, P99H, P99E, P99D, E102H/E103H, E102D/E103D, F103H/F104H, F103E/F104E, F103D/F104D, F103V/F104V, F103I/F104I, K104H/K105H, K104E/K105E, K104D/K105D, L111H/L112H/M112H, L111E/L112E/M112E, L111D/L112E/M112D, V113H/V114H/L114H, V113E/V114E/L114E, V113D/V114D/L114D, R114H/R115H/G115H, R114E/R115E/G115E, R114D/R115D/G115D, K116H/K117H, K116E/K117E, and K116D/K117D, wherein the “/” indicates different alternative mutation change at the corresponding amino acid residue position across SIRPα, SIRPβ and SIRPγ IgV domains, due to different parent amino acid residues and/or position numbering for the mutation at the corresponding position, according to the SIRPα, SIRPβ and SIRPγ IgV amino acid sequences provided in SEQ ID NO: 1 to 21

18.-44. (canceled)

45. A multimer polypeptide comprising two or more serially linked IgV extracellular domain of signal regulatory protein (SIRP), wherein the multimer polypeptide comprises one or more of the variant of claim 1, and wherein the two or more IgV extracellular domain of signal regulatory protein (SIRP) comprise the same or different amino acid sequence and/or mutation.

46. A fusion polypeptide comprising a variant of claim 1.

47.-49. (canceled)

50. A fusion protein comprising at least one fusion polypeptide of claim 4.

51. (canceled)

52. The fusion protein of claim 50, comprising one, two, three or four of the variant of claim 1.

53.-64. (canceled)

65. An antibody fusion protein comprising: 1) an antibody binding to a non-CD47 antigen comprising two heavy chains and two light chains, wherein the two Fc region of the two heavy chains form a homodimeric Fc, and 2) a SIRP IgV domain, wherein the SIRP IgV domain comprises one, two, three or more of the variant of claim 1, and is linked preferably through a linker to the N-terminal of the heavy chain and/or the light chain of the antibody.

66.-70. (canceled)

71. An antibody fusion protein comprising: 1) a first polypeptide comprising a SIRP IgV domain and a Fc region, wherein the SIRP IgV domain comprises one, two, three, four or more of the variant of claim 1, and is linked to the N-terminal of the Fc region, 2) a heavy chain of an antibody and a full light chain of an antibody, wherein the antibody heavy chain and the light chain pair to form an half-antibody that binds to a non-CD47 antigen, and 3) the Fc region of the first polypeptide and the antibody heavy chain form a heterodimeric Fc.

72.-74. (canceled)

75. An antibody fusion protein comprising: 1) a first polypeptide comprising a first SIRP IgV domain and a Fc region, wherein the SIRP IgV domain comprises one, two, three or more IgV extracelluar domain of SIRPα, SIRPβ and/or SIRPγ and is linked to the N-terminal of the Fc region, 2) a heavy chain of an antibody and a light chain of an antibody, wherein the antibody heavy chain and the light chain pair to form an half-antibody that binds to a non-CD47 antigen, 3) a second SIRP IgV domain, wherein the second SIRP IgV domain comprises one, two, three or more IgV extracelluar domain of SIRPα, SIRPβ and/or SIRPγ and is linked preferably through a linker to the N-terminal of the antibody heavy chain and/or light chain, and 4) the Fc region of the first polypeptide and the antibody heavy chain form a heterodimeric Fc; wherein the first and/or the second SIRP IgV domains comprise one, two, three or more of the variant of claim 1, wherein the two, three, or more of the variants are the same or different.

76.-77. (canceled)

78. The antibody fusion protein of claim 75, wherein the first SIRP IgV domain comprise an amino acid sequene selected from the group consisting of SEQ ID NO: 3 to 21 and the second SIRP IgV domain comprises one IgV extracelluar domain of SIRPα, SIRPβ or SIRPγ comrpising a mutation selected from the group consisting of K53H, K53H+K68H, K53H+Q52H, K53H+K68H+Q52H, K53H+I31E, K53H+I31D, K53H+L31E, K53H+Q37H, K53H+L37H, K53H+H101D, K53H+N101D, K53H+L37Q, K53H+N101D+L37Q, K53H+N101D+L31E, K53H+N101D+L31D, K53H+L37Q+L31E, K53H+L37Q+L31D, K53H+N101D+L37Q+L31E, and K53H+N101D+L37Q+L31D.

79.-85. (canceled)

86. A Fab fusion protein comprising: 1) a Fab domain comprising a Fd chain comprising VH-CH1 and a light chain comprising VL-CL from the N-terminal to C-terminal, wherein the Fab domain binds to a non-CD47 antigen and 2) a SIRP IgV domain, wherein the SIRP IgV domain comprises one, two, three, four or more IgV extracellular domain of SIRPα, SIRPβ and/or SIRPγ, and is linked to the N-terminal and/or C-terminal of the Fd chain and/or light chain of the Fab domain; wherein the SIRP IgV domain comprises one, two, three, four or more of the variant of claim 1.

87.-89. (canceled)

90. A protein drug conjugate comprising a variant claim 1.

91.-105. (canceled)

106. A synthetic receptor comprising an antigen binding domain, a transmembrane domain and an intracellular signaling domain, wherein the antigen binding domain comprises a variant of claim 1.

107.-115. (canceled)

116. A nucleic acid comprising a sequence encoding a variant of claim 1.

117.-119. (canceled)

120. A modified cell comprising a nucleic acid of claim 1.

121.-130. (canceled)

131. A pharmaceutical composition comprising the variant of claim 1, and a pharmaceutically acceptable carrier.

132. A method of treating a CD47-expressing disease in a mammal comprising administering an effective amount of the variant domain of claim 1, to a mammal in need thereof.

133.-139. (canceled)

140. The method of claim 132, wherein the CD47-expressing disease is a disease of cancer, fibrosis, atherosclerosis, inflammation or senescence.

141.-143. (canceled)

144. A method of combination therapy in human comprising administering a therapeutically effective amount of the of claim 1, and a therapeutically effective amount of another therapy.

145. The method of combination therapy of claim 144, wherein another therapy comprises administration of one or more of chemotherapy, molecular targeted therapy, biologics therapy, immunotherapy and radiotherapy.

146.-147. (canceled)

148. An antibody fusion protein comprising: 1) a partial heavy chain comprising CH1-hinge-CH2-CH3 from the N-terminal to C-terminal, 2) a partial light chain comprising CL domain, wherein the CL domain pairs with the CH1 domain of the partial heavy chain with disulfide bond, 3) the Fc region of the two partial heavy chain form a homodimeric or heterodimeric Fc, and 4) a SIRP IgV domain is linked preferably through a linker to the N-terminal of the partial light chain and/or the partial heavy chain, and wherein the SIRP IgV domain comprises one, two, three, four or more variant of claim 1.
